SEQUENCE LISTING (25 sequences)

SEQ ID NO: 1 - protein, 272 aa, Homo sapiens
IAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAM

SEQ ID NO: 2 - protein, 293 aa, Artificial Sequence
  Description of Artificial Sequence Synthetic pET15S protein containing PYK2
MGSSHHHHHHSSGLVPRGSHMIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAM

SEQ ID NO: 3 - DNA, 816 bp, Homo sapiens
attgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatg

SEQ ID NO: 4 - DNA, 1050 bp, Artificial Sequence
  Description of Artificial Sequence Synthetic pET15S nucleotide sequence
tctagaaataattttgtttaactttaagaaggagatataccatgggcagcagccatcatcatcatcatcacagcagcggcctggtgccgcgcggcagccatatgattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatgtaggtcgactagagcctgcagtctcgaccatcatcatcatcatcattaataaaagggcgaattccagcacactggcggccgttactagtggatccggctgctaacaaagcccgaaaggaagctgagttgg

SEQ ID NO: 5 - DNA, 33 bp, Artificial Sequence
  Description of Artificial Sequence Primer
tccacagcatatgattgcccgtgaagatgtggt

SEQ ID NO: 6 - DNA, 34 bp, Artificial Sequence
  Description of Artificial Sequence Primer
ctctcgtcgacctacatggcaatgtccttctcca

SEQ ID NO: 7 - DNA, 391 bp, Artificial Sequence
  CDS (108)..(170)
  Description of Artificial Sequence Synthetic pET15S multi-cloning site nucleotide sequence
agatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgggatccggaattcaaaggcctacgtcgactagagcctgcagtctcgaccatcatcatcatcatcattaataaaagggcgaattccagcacactggcggccgttactagtggatccggctgctaacaaagcccgaaaggaagctgagttggctgctgccaccgctgagcaataactagcataaccccttggggcctctaaacgggtcttgaggggttttttg

SEQ ID NO: 8 - protein, 21 aa, Artificial Sequence
  Description of Artificial Sequence Synthetic pET15S multi-cloning site peptide sequence
MGSSHHHHHHSSGLVPRGSHM

SEQ ID NO: 9 - protein, 310 aa, Unknown Organism
  Description of Unknown Organism FAK tyrosine kinase sequence
EIQRERIELGRCIGEGQFGDVHQGIYMSPENPALAVAIKTCKNCTSDSVREKFLQEACHYTSLHWNWCRYISDPNVDACPDPRNAELTMRQFDHPHIVKLIGVITENPVWIIMELCTLGELRSFLQVRKYSLDLASLILYAYQLSTALAYLESKRFVHRDIAARNVLVSSNDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKAQQEERMRMESR

SEQ ID NO: 10 - protein, 273 aa, Unknown Organism
  Description of Unknown Organism SRC tyrosine kinase sequence
EIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 11 - protein, 271 aa, Unknown Organism
  Description of Unknown Organism HCK tyrosine kinase sequence
EIPRESLKLEKKLGAGQFGEVWMATYNKHTKVAVKTMKPGSMSVEAFLAEANVMKTLQHDKLVKLHAVVTKEPIYIITEFMAKGSLLDFLKSDEGSKQPLPKLIDFSAQIAEGMAFIEQRNYIHRDLRAANILVSASLVCKIADFGLARVIEDNEYTAREGAKFPIKWTAPEAINFGSFTIKSDVWSFGILLMEIVTYGRIPYPGMSNPEVIRALERGYRMPRPENCPEELYNIMMRCWKNRPEERPTFEYIQSVLDDFYTATESQYEEIP

SEQ ID NO: 12 - protein, 263 aa, Unknown Organism
  Description of Unknown Organism LCK tyrosine kinase sequence
EVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTAT

SEQ ID NO: 13 - protein, 263 aa, Unknown Organism
  Description of Unknown Organism ABL1 tyrosine kinase sequence
EMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQ

SEQ ID NO: 14 - protein, 260 aa, Unknown Organism
  Description of Unknown Organism CSK tyrosine kinase sequence
ALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHEL

SEQ ID NO: 15 - protein, 295 aa, Unknown Organism
  Description of Unknown Organism TEK tyrosine kinase sequence
VLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFT

SEQ ID NO: 16 - protein, 291 aa, Unknown Organism
  Description of Unknown Organism KDR tyrosine kinase sequence
EFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKVAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANA

SEQ ID NO: 17 - protein, 290 aa, Unknown Organism
  Description of Unknown Organism FGFR1 tyrosine kinase sequence
ELPRDRLVLGKPLGEGAFGQVVLAEAIGLDKDKPNRVTKVAVKMLKSDATEKDLSDLISEMEMMKMIGKHKNIINLLGACTQDGPLYVIVEYASKGNLREYLQARRPPGLEYSYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALT

SEQ ID NO: 18 - protein, 294 aa, Unknown Organism
  Description of Unknown Organism INSR tyrosine kinase sequence
EVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 19 - protein, 294 aa, Homo sapiens
EVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLLELMRMCWQYNPKMRPSFLEIISSIKEEMEPGFREVSFYYSEENK

SEQ ID NO: 20 - protein, 278 aa, Unknown Organism
  Description of Unknown Organism EPHB2 tyrosine kinase sequence
EIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKA

SEQ ID NO: 21 - protein, 283 aa, Unknown Organism
  Description of Unknown Organism EGFR tyrosine kinase sequence
ILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMH

SEQ ID NO: 22 - protein, 6 aa, Artificial Sequence
  Description of Artificial Sequence 6-His tag
HHHHHH

SEQ ID NO: 23 - protein, 8 aa, Homo sapiens
DIYAEIPD

SEQ ID NO: 24 - protein, 4 aa, Artificial Sequence
  Description of Artificial Sequence Synthetic pET15S peptide fragment
GSHM

SEQ ID NO: 25 - protein, 7 aa, Homo sapiens
IYAEIPD

(19) United States
(12) Patent Application Publication
(10) Pub. No.: US 2005/0170431 A1
(43) Pub. Date: Aug. 4, 2005

(54) PYK2 crystal structure and uses

(75) Inventors: Prabha Ibrahim, Mountain View, CA (US); Heike Krupka, Hayward, CA (US); Abhinav Kumar, Pleasant Hill, CA (US); Michael V. Milburn, Emeryville, CA (US); Yoshihisa Suzuki, El Sobrante, CA (US)

Correspondence Address: FOLEY & LARDNER, P.O. BOX 80278, SAN DIEGO, CA, 92138-0278, US

(73) Assignee: Plexxikon, Inc.

(21) Appl. No.: 10/789,818

(22) Filed: Feb. 27, 2004

Related U.S. Application Data
(60) Provisional application No. 60/451,101, filed on Feb. 28, 2003.

(51) Int. Cl. G01N 33/53; C07K 16/40

(52) U.S. Cl. 435/7.1; 530/388.26

(57) ABSTRACT

A crystal structure of PYK2 is described that was determined by X-ray crystallography. The use of PYK2 crystals and strucural information can, for example, be used for identifying molecular scaffolds and for developing ligands that bind to and modulate PYK2.

### CROSS-REFERENCE TO RELATED PATENT APPLICATIONS

This application claims the benefit of Ibrahim et al., U.S. Provisional Application 60/451,101, filed Feb. 28, 2003, which is incorporated herein by reference in its entirety, including drawings.

### BACKGROUND OF THE INVENTION

This invention relates to the field of development of ligands for protein tyrosine kinase 2 (PYK2) and to the use of crystal structures of PYK2. The information provided is intended solely to assist the understanding of the reader. None of the information provided nor references cited is admitted to be prior art to the present invention.

Cellular signal transduction is a fundamental mechanism whereby external stimuli that regulate diverse cellular processes are relayed to the interior of cells. One of the key biochemical mechanisms of signal transduction involves the reversible phosphorylation of tyrosine residues on proteins. The phosphorylation state of a protein is modified through the reciprocal actions of tyrosine phosphatases (TPs) and tyrosine kinases (TKs), including receptor tyrosine kinases and non-receptor tyrosine kinases.

Receptor tyrosine kinases (RTKs) belong to a family of transmembrane proteins and have been implicated in cellular signaling pathways. The predominant biological activity of some RTKs is the stimulation of cell growth and proliferation, while other RTKs are involved in arresting growth and promoting differentiation. In some instances, a single tyrosine kinase can inhibit, or stimulate, cell proliferation depending on the cellular environment in which it is expressed.

RTKs are composed of at least three domains: an extra-cellular ligand binding domain, a transmembrane domain and a cytoplasmic catalytic domain that can phosphorylate tyrosine residues. Ligand binding to membrane-bound receptors induces the formation of receptor dimers and allosteric changes that activate the intracellular kinase domains and result in the self-phosphorylation (autophosphorylation and/or transphosphorylation) of the receptor on tyrosine residues. Individual phosphotyrosine residues of the cytoplasmic domains of receptors may serve as specific binding sites that interact with a host of cyto-plasmic signaling molecules, thereby activating various signal transduction pathways.

The intracellular, cytoplasmic, non-receptor protein tyrosine kinases do not contain a hydrophobic transmembrane domain or an extracellular domain and share non-catalytic domains in addition to sharing their catalytic kinase domains. Such non-catalytic domains include the SH2 domains and SH3 domains. The non-catalytic domains are thought to be important in the regulation of protein-protein interactions during signal transduction.

A central feature of signal transduction is the reversible phosphorylation of certain proteins. Receptor phosphorylation stimulates a physical association of the activated receptor with target molecules, which either are or are not phosphorylated.

Some of the target molecules such as phospholipase Cγ are in turn phosphorylated and activated. Such phosphorylation transmits a signal to the cytoplasm. Other target molecules are not phosphorylated, but assist in signal transmission by acting as adapter molecules for secondary signal transducer proteins. For example, receptor phosphorylation and the subsequent allosteric changes in the receptor recruit the Grb-2/SOS complex to the catalytic domain of the receptor where its proximity to the membrane allows it to activate ras.

The secondary signal transducer molecules generated by activated receptors result in a signal cascade that regulates cell functions such as cell division or differentiation. Reviews describing intracellular signal transduction include Aaronson, Science 254:1146-1153, 1991; Schlessinger, Trends Biochem. Sci., 13:443-47, 1988; and Ullrich and Schlessinger, Cell, 61:203-212, 1990.

Signal transduction pathways that regulate ion channels (e.g., potassium channels and calcium channels) involve G proteins which function as intermediaries between receptors and effectors. Gilman, Ann. Rev. Biochem., 56:615-649 (1987); Brown and Bimbaumer, Ann. Rev. Physiol., 52: 197-213 (1990). G-coupled protein receptors are receptors for neurotransmitters, ligands that are responsible for signal production in nerve cells as well as for regulation of proliferation and differentiation of nerves and other cell types. Neurotransmitter receptors exist as different subtypes which are differentially expressed in various tissues and neurotransmitters such as acetylcholine evoke responses throughout the central and peripheral nervous systems.

The muscarinic acetylcholine receptors play important roles in a variety of complex neural activities such as learning, memory, arousal and motor and sensory modulation. These receptors have also been implicated in several central nervous system disorders such as Alzheimer's disease, Parkinson's disease, depression and schizophrenia.

Some agents that are involved in a signal transduction pathway regulating one ion channel, for example a potassium channel, may also be involved in one or more other pathways regulating one or more other ion channels, for example a calcium channel. Dolphin, Ann. Rev. Physiol., 52:243-55 (1990); Wilk-Blaszczak et al., Neuron, 12: 109-116 (1994). Ion channels may be regulated either with or without a cytosolic second messenger. Hille, Neuron, 9:187-195 (1992). One possible cytosolic second messenger is a tyrosine kinase. Huang et al., Cell, 75:1145-1156 (1993), incorporated herein by reference in its entirety, including any drawings.

The receptors involved in the signal transduction pathways that regulate ion channels are ultimately linked to the ion channels by various intermediate events and agents. For example, such events include an increase in intracellular calcium and inositol triphosphate and production of endothelin. Frucht, et al., Cancer Research, 52:1114-1122 (1992); Schrey, et al., Cancer Research, 52:1786-1790 (1992). Intermediary agents include bombesin, which stimulates DNA synthesis and the phosphorylation of a specific protein kinase C substrate. Rodriguez-Pena, et al., Biochemical and Biophysical Research Communication, 140(1):379-385 (1986); Fisher and Schonbrunn, J. Biol. Chem., 263(6):2208-2816 (1988).

Focal adhesion kinase (FAK) is a cytoplasmic protein tyrosine kinase localized to focal adhesions that is known to associate with two Src family kinases. Schaller, et al., Proc. Natl. Acad. Sci. U.S.A., 89:5192-5196 (1992), incorporated herein by reference in its entirety, including any drawings; Cobb et al., Mol. Cell. Biol., 14(1):147-155 (1994). The proteins associated with the cytoplasmic surface of adhesion molecules are reviewed in Gumbiner, Neuron, 11:551-564 (1993).

FAK may regulate interactions of integrins, agonist receptors, and/or stress fibers. Shattil et al., J. Biol. Chem., 269(20):14738-14745 (1994); Ridley and Hall, The EMBO Journal, 13(11):2600-2610 (1994). FAK does not contain SH2 or SH3 domains and the amino acid sequence of FAK is highly conserved among birds, rodents and man.

In some cells the C-terminal domain of FAK is expressed autonomously as a 41 kDa protein called FRNK and the 140 C-terminal residues of FAK contain a focal adhesiori targeting (FAT) domain. The cDNA's encoding FRNK are given in Schaller et al., Mol. Cell. Biol., 13(2):785-791 (1993), incorporated herein by reference in its entirety, including any drawings. The FAT domain was identified and said to be required for localization of FAK to cellular focal adhesions in Hilderbrand et al., J. Cell Biol., 123(4):993-1005 (1993).

The non-receptor tyrosine kinase, PYK2, is activated by binding of ligand to G-coupled protein receptors such as bradykinin and acetylcholine. PYK2 has a predicted molecular weight of 111 kD and contains five domains: (1) a relatively long N-terminal domain; (2) a kinase catalytic domain; (3) a proline rich domain; (4) another proline rich domain; and (5) a C-terminal focal adhesion targeting (FAT) domain. PYK2 does not contain a SH2 or SH3 domain.

The FAT domain of PYK2 has 62% similarity to the FAT domain of another non-receptor tyrosine kinase, FAK, which is also activated by G-coupled proteins. The overall similarity between PYK2 and FAK is 52%. PYK2 is expressed principally in neural tissues, although expression can also be detected in hematopoietic cells at early stages of develop-ment and in some tumor cell lines. The expression of PYK2 does not correspond with the expression of FAK.

PYK2 is also known as Cell Adhesion Kinase β (CAK β) and Related Adhesion Focal Tyrosine Kinase (RAFTK). Nucleotide and amino acid sequences for PYK2 are described in a set of related patents, including U.S. Pat. Nos. 8,837,815; 5,837,524; and Patent Publication U.S. 2002/0048782, which also provided additional information on PYK2 and a related protein, FAK, including some of the information described below. Each of these documents describes nucleotide and amino acid sequences for PYK2. U.S. Pat. No. 5,837,524 describes a method of screening for agents “able to promote or disrupt the interaction” between “a PYK2 polypeptide and a natural binding partner (NBP).” (Col. 8, lines 60-67.) Patent Publication U.S. 2002/0048782 provides examples describing cloning and the testing of certain properties of PYK2. Each of these patents and patent publication are incorporated by reference herein in their entireties, including drawings.

PYK2 is believed to regulate the activity of potassium channels in response to neurotransmitter signalling. PYK2 enzymatic activity is positively regulated by phosphorylation on tyrosine and results in response to binding of bradykinin, TPA, calcium ionophore, carbachol, TPA+ forskolin, and membrane depolarization. The combination of toxins known to positively regulate G-coupled receptor signalling (such as pertusis toxin, cholera toxins, TPA and bradykinin) increases the phosphorylation of PYK2. Activated PYK2 phosphorylates RAK, a delayed rectifier type potassium channel, and thus suppresses RAK activity. In the same system, FAK does not phosphorylate RAK.

Further, integrin-linked signaling is important for regulating cell adhesion and motility. (Hynes, R. (2002) Integrins: bidirectional, allosteric signaling machines. Cell, 110, 673-687.) The FAK and PYK2 tyrosine kinases are key mediators of integrin-dependent signals. (Hauck et al. (2000) Focal adhesion kinase functions as a receptor-proximal signaling component required for directed cell migration. Immunol Res, 21, 293-303.) Both FAK and PYK2 mediate cytoskeletal rearrangements as a consequence of integrin ligation. FAK, which localizes to focal adhesions, is activated by binding of cell-surface integrins to the extracellular matrix. In response to external stimuli, growth factors associate with integrins, and FAK also becomes phosphorylated in response to growth factors. (Sieg, et al. (2000) FAK integrates growth-factor and integrin signals to promote cell migration. Nat Cell Biol, 2, 249-256.) In addition to its role in regulating the cytoskeleton and cell movements, FAK also helps to coordinate these processes with growth signals and cellular survival.

By contrast, PYK2 is localized to the sites of cell-cell contacts, and becomes activated in response to calcium mobilization. (Lev, et al. (1995) Protein tyrosine kinase PYK2 involved in Ca(2+)-induced regulation of ion channel and MAP kinase functions. Nature, 376, 737-745.) Indeed, whereas FAK appears to mediate cellular survival, PYK2 activation leads to apoptosis in fibroblasts. (Xiong, W. and Parsons, J. T. (1997) Induction of apoptosis after expression of PYK2, a tyrosine kinase structurally related to focal adhesion kinase. J Cell Biol, 139, 529-539.) In monocytes and osteoclasts, PYK2 localizes to the podosome, a cellular protrusion that contacts the extracellular matrix and mediates adhesion and motility in these cell types. (Duong et al. (1998) PYK2 in osteoclasts is an adhesion kinase, localized in the sealing zone, activated by ligation of alpha(v)beta3 integrin, and phosphorylated by src kinase. J Clin Invest, 102, 881-892; Lakkakorpi et al. (1999) Stable association of PYK2 and p130(Cas) in osteoclasts and their co-localization in the sealing zone. J Biol Chem, 274, 4900-4907.)

In spite of the different biological functions, FAK and PYK2 are the only members of the FAK family of tyrosine kinases, and they share 45% sequence identity overall, with higher homology in the kinase catalytic domain (60%). (Lev et al. (1995) Nature, 376, 737-745; Sasaki et al. (1995) Cloning and characterization of cell adhesion kinase beta, a novel protein-tyrosine kinase of the focal adhesion kinase subfamily. J Biol Chem, 270, 21206-21219.) Furthermore, most of the key regulatory sites are highly conserved. In the N-terminus is a large integrin-binding domain. In the C-terminus is the so-called FAT (focal adhesion targeting) domain that mediates subcellular localization via binding sites for the cytoskeleton-associated proteins paxillin and talin. The kinase catalytic domain is in the center of the proteins. In addition, proline-rich regions in the C-terminus serve to bind to the SH3 domains of the adaptor proteins CAS and GRAF. (Hildebrand et al. (1996) An SH3 domain-containing GTPase-activating protein for Rho and Cdc42 associates with focal adhesion kinase. Mol Cell Biol, 16, 3169-3178; Polte, T. R. and Hanks, S. K. (1995) Interaction between focal adhesion kinase and Crk-associated tyrosine kinase substrate p130Cas. Proc Natl Acad Sci USA, 92, 10678-10682.)

The primary autophosphorylation site (Y397 in FAK, Y402 in PYK2, just upstream of the catalytic domain) serves as a binding site for the SH2 domain of a Src-family tyrosine kinase. (Dikic et al. (1996) A role for Pyk2 and Src in linking G-protein-coupled receptors with MAP kinase activation. Nature, 383, 547-550.) This site is also a substrate for the Src kinase. Additional tyrosine phosphorylation events occur at residues within the catalytic domain (Y576, Y577 in FAK, Y579, Y580 in PYK2) whose function is unclear, and at a C-terminal site (Y925 in FAK, Y881 in PYK2) that serves as binding site for the SH2 domain of GRB2. (Schlaepfer et al. (1999) Signaling through focal adhesion kinase. Prog Biophys Mol Biol, 71, 435-478.) In addition to assembling a variety of proteins, FAK and PYK2 also play important roles by phosphorylating key substrates such as paxillin and CAS. (Bellis et al. (1995) Characterization of tyrosine phosphorylation of paxillin in vitro by focal adhesion kinase. J Biol Chem, 270, 17437-17441; Li, X. and Earp, H. S. (1997) Paxillin is tyrosine-phosphorylated by and preferentially associates with the calcium-dependent tyrosine kinase in rat liver epithelial cells. J Biol Chem, 272, 14341-14348.) Tyrosine phosphorylation of paxillin and CAS creates a new binding site for SH2 adaptor proteins. For example, paxillin binds to and is phosphorylated by PYK2 in hematopoietic cells. (McShan et al. (2002) Csk homologous kinase associates with RAFTK/Pyk2 in breast cancer cells and negatively regulates its activation and breast cancer cell migration. Internat. J. Oncology 21:197-205.)

Furthermore, expression of PYK2 and FAK was observed in breast cancer cells, and it was reported that PYK2 participates in intracellular signaling upon heregulin (HRG) stimulation and promotes breast carcinoma invasion. CHK acted as a negative regulator of PYK2, significantly reducing the migration of PYK2 expressing breast cancer cells. (McShan et al. (2002) Internat. J. Oncology 21:197-205.)

Methods of identifying a compound that binds to and/or modulates the activity of PYK2 are described in Duong et al., PCT/US98/02797, WO 98/35056, where the method involves contacting the compound and PYK2 and determining if binding has occurred. If binding has occurred, the activity of the bound PYK2 can be compared to the activity of PYK2 which is not bound to the compound to determine if the compound modulates PYK2 activity. (p.2, lines 9-15) The compounds identified are indicated to be useful in the prevention or teatment of osteoporosis, inflammation, and other conditions dependent on monocyte migration and invasion activities. (p.3, lines 1-5) This application is hereby incorporated by reference in its entirety.

### SUMMARY OF THE INVENTION

The present invention concerns structural information about PYK2 kinase, crystals of PYK2 kinases with and without binding compounds, and the use of the PYK2 kinase crystals and structural information about the PYK2 kinase to develop PYK2 ligands, e.g., inhibitors.

Thus, in a first aspect, the invention concerns a method for determining the orientation of compounds that bind to PYK2 and/or identifying binding compounds by determining the orientation of at least one compound bound to PYK2 in co-crystals of PYK2 with binding compound. The method also characterizes the binding of a PYK2 binding compound bound to PYK2. In particular embodiments, the method can also involve one or more of: identifying as molecular scaffolds one or more compounds that bind weakly (with low or very low affinity) to a binding site of PYK2 kinase and have molecular weight less than 350 daltons; determining activity of the compounds or molecular scaffolds against PYK2 (activity can also be determined against 1, 2, 3, or more additional kinases; scaffolds preferably have low activity); determining the orientation of at least one molecular scaffold in co-crystals with PYK2 kinase; identifying chemical structures of one or more of the molecular scaffolds that, when modified, alter the binding affinity or binding specificity or both between the molecular scaffold and the PYK2 kinase; synthesizing or otherwise obtaining a ligand in which one or more of the chemical structures of the molecular scaffold is modified to provide a ligand that binds to the PYK2 kinase with altered binding affinity or binding specificity or both. Thus, the invention provides a method for identifying or developing PYK2 ligands, e.g., by identifying derivatives of PYK2 binding compounds, which may be molecular scaffolds, that have greater affinity and/or greater specificity for PYK2 than the parent compound. For example, the method can involve determining the binding orientation, identifying one or more chemical structures of one or more compounds that, when modified, alter the binding affinity and/or specificity; and synthesizing or otherwise obtaining a ligand in which one or more of those chemical structures is modified to provide a ligand that binds to PYK2 kinase with altered binding affinity or binding specificity or both. The method can also include identifying a molecular scaffold that binds to PYK2. Highly preferably the modified compound (ligand) also has altered activity (i.e., altered effect on the activity of PYK2 kinase).

The terms “PYK2 kinase” and “PYK2” mean an enzymatically active kinase that contains a portion at least 50 amino acid residues in length with greater than 90% amino acid sequence identity to at least a portion of PYK2 kinase domain (SEQ ID NO.: 1), for a maximal alignment over an equal length segment; or that contains a portion with greater than 90% amino acid sequence identity to SEQ ID NO.: 1 that retains binding to ATP. Preferably the sequence identity is at least 95, 97, 98, 99, or even 100% with SEQ ID NO. 1. Preferably the identity is over a portion of SEQ ID NO: 1 that is at least 100, 150, 200, 250, or 272 amino acid in length.

The term “PYK2 kinase domain” refers to a reduced length PYK2 (i.e., shorter than a full-length PYK2 by at least 100 amino acids at each of the N-terminus and the C-terminus) that includes the kinase catalytic region in PYK2, which is located near the center of the full-length molecule. Highly preferably for use in this invention, the kinase domain retains kinase activity, preferably at least 50% the level of kinase activity as compared to the native PYK2, more preferably at least 60, 70, 80, 90, or 100% of the native activity in a competitive kinase assay with ATP as a substrate and ATPγS as competitive inhibitor. An example is the PYK2 kinase domain of SEQ ID NO: 1.

As used herein, the terms “ligand” and “modulator” are used equivalently to refer to a compound that modulates the activity of a target biomolecule, e.g., an enzyme such as a kinase. Generally a ligand or modulator will be a small molecule, where “small molecule refers to a compound with a molecular weight of 1500 daltons or less, or preferably 1000 daltons or less, 800 daltons or less, or 600 daltons or less. Thus, an “improved ligand” is one that possesses better pharmacological and/or pharmacokinetic properties than a reference compound, where “better” can be defined by a person for a particular biological system or therapeutic use. In terms of the development of ligands from scaffolds, a ligand is a derivative of a scaffold.

In the context of binding compounds, molecular scaffolds, and ligands, the term “derivative” or “derivative compound” refers to a compound having a chemical structure that contains a common core chemical structure as a parent or reference compound, but differs by having at least one structural difference, e.g., by having one or more substituents added and/or removed and/or substituted, and/or by having one or more atoms substituted with different atoms. Unless clearly indicated to the contrary, the term “derivative” does not mean that the derivative is synthesized using the parent compound as a starting material or as an intermediate, although in some cases, the derivative may be synthesized from the parent.

Thus, the term “parent compound” refers to a reference compound for another compound, having structural features continued in the derivative compound. Often but not always, a parent compound has a simpler chemical structure than the derivative.

By “chemical structure” or “chemical substructure” is meant any definable atom or group of atoms that constitute a part of a molecule. Normally, chemical substructures of a scaffold or ligand can have a role in binding of the scaffold or ligand to a target molecule, or can influence the three-dimensional shape, electrostatic charge, and/or conformational properties of the scaffold or ligand.

The term “binds” in connection with the interaction between a target and a potential binding compound indicates that the potential binding compound preferentially associates with the target to a statistically significant degree as compared to association with proteins generally (i.e., non-specific binding). Thus, the term “binding compound” refers to a compound that has such a statistically significant association with a target molecule. Preferably a binding compound interacts with a specified target with a dissociation constant (k_{d}) of 1 mM or less. A binding compound can bind with “low affinity”, “very low affinity”, “extremely low affinity”, “moderate affinity”, “moderately high affinity”, or “high affinity” as described herein.

In the context of compounds binding to a target, the term “greater affinity” indicates that the compound binds more tightly than a reference compound, or than the same compound in a reference condition, i.e., with a lower dissociation constant. In particular embodiments, the greater affinity is at least 2, 3, 4, 5, 8, 10, 50, 100, 200, 400, 500, 1000, or 10,000-fold greater affinity.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules, e.g., in the case of PYK2, other kinases or even other type of enzymes. In particular embodiments, the greater specificity is at least 2, 3, 4, 5, 8, 10, 50, 100, 200, 400, 500, or 1000-fold greater specificity.

As used in connection with binding of a compound with PYK2, the term “interact” indicates that the distance from a bound compound to a particular amino acid residue will be 5.0 angstroms or less, or 6 angstroms or less with one water molecule coordinated between the compound and the residue, or 9 angstroms or less with two water molecules coordinated between the compound and the residue. In particular embodiments, the distance from the compound to the particular amino acid residue is 4.5 angstroms or less, 4.0 angstroms or less, or 3.5 angstroms or less. Such distances can be determined, for example, using co-crystallography, or estimated using computer fitting of a compound in a PYK2 active site.

Reference to particular amino acid residues in PYK2 polypeptide residue number is defined by the numbering provided in Lev et al. (1995) “Protein tyrosine kinase PYK2 involved in Ca(2+)-induced regulation of ion channel and MAP kinase functions” Nature 376:737-745.

In a related aspect, the invention provides a method for developing ligands specific for PYK2 kinase, where the method involves determining whether a derivative of a compound that binds to a plurality of kinases has greater specificity for the PYK2 kinase than the parent compound with respect to other kinases. In particular embodiments, the method also involves identifying such a compound that binds to a plurality of kinases.

As used herein in connection with binding compounds or ligands, the term “specific for PYK2 kinase”, “specific for PYK2” and terms of like import mean that a particular compound binds to the particular PYK2 kinase to a statistically greater extent than to other kinases that may be present in a particular organism. Also, where biological activity other than binding is indicated, the term “specific for a PYK2 kinase” indicates that a particular compound has greater biological activity associated with binding PYK2 than to other kinases. Preferably, the specificity is also with respect to other biomolecules (not limited to kinases) that may be present from an organism.

In another aspect, the invention provides a method for obtaining improved ligands binding to PYK2, where the method involves identifying a compound that binds to PYK2, determining whether that compound interacts with one or more of PYK2 residues 503, 505, 457, 488, 567, and 554, and determining whether a derivative of that compound binds to the PYK2 kinase with greater affinity or greater specificity or both than the parent binding compound. Binding with greater affinity or greater specificity or both than the parent compound indicates that the derivative is an improved ligand. This process can also be carried out in successive rounds of selection and derivatization and/or with multiple parent compounds to provide a compound or compounds with improved ligand characteristics. Likewise, the derivative compounds can be tested and selected to give high selectivity for the PYK2 kinase, or to give cross-reactivity to a particular set of targets, for example to a subset of kinases that includes PYK2. Certain compounds interact with the specified residues as 503, 505 (direct interacting), 457, 488, 567 (interact through 1 water), and 554 (interact through 2 waters). In particular embodiments, a molecular scaffold, binding compound, or ligand interacts with at least residues 503 and 505; residues 503 and 505 and at least one of residues 457, 488, and 567; at least residues 503, 505, 457, 488, and 567.

By “molecular scaffold” or “scaffold” is meant a simple target binding molecule to which one or more additional chemical moieties can be covalently attached, modified, or eliminated to form a plurality of molecules with common structural elements. The moieties can include, but are not limited to, a halogen atom, a hydroxyl group, a methyl group, a nitro group, a carboxyl group, or any other type of molecular group including, but not limited to, those recited in this application. Molecular scaffolds bind to at least one target molecule, preferably to a plurality of molecules in a target family, e.g., a protein family. Preferred target molecules include enzymes and receptors, as well as other proteins. Preferred characteristics of a scaffold can include binding at a target molecule binding site such that one or more substituents on the scaffold are situated in binding pockets in the target molecule binding site; having chemically tractable structures that can be chemically modified, particularly by synthetic reactions, e.g., so that a combinatorial library can be easily constructed; having chemical positions where moieties can be attached that do not interfere with binding of the scaffold to a protein binding site, such that the scaffold or library members can be modified to form ligands, to achieve additional desirable characteristics, e.g., enabling the ligand to be actively transported into cells and/or to specific organs, or enabling the ligand to be attached to a chromatography column for additional analysis. Thus, a molecular scaffold is an identified target binding molecule prior to modification to improve binding affinity and/or specificity, or other pharmacalogic properties.

The term “scaffold core” refers to the core structure of a molecular scaffold onto which various substituents can be attached. Thus, for a number of scaffold molecules of a particular chemical class, the scaffold core is common to all the scaffold molecules. In many cases, the scaffold core will consist of or include one or more ring structures.

By “binding site” is meant an area of a target molecule to which a ligand can bind non-covalently. Binding sites embody particular shapes and often contain multiple binding pockets present within the binding site. The particular shapes are often conserved within a class of molecules, such as a protein family. Binding sites within a class also can contain conserved structures such as, for example, chemical moieties, the presence of a binding pocket, and/or an electrostatic charge at the binding site or some portion of the binding site, all of which can influence the shape of the binding site.

By “binding pocket” is meant a specific volume within a binding site. A binding pocket can often be a particular shape, indentation, or cavity in the binding site. Binding pockets can contain particular chemical groups or structures that are important in the non-covalent binding of another molecule such as, for example, groups that contribute to ionic, hydrogen bonding, or van der Waals interactions between the molecules.

By “orientation”, in reference to a binding compound bound to a target molecule is meant the spatial relationship of the binding compound (which can be defined by reference to at least some of its consitituent atoms) to the binding site and/or atoms of the target molecule at least partially defining the binding site, typically including one or more binding pockets and/or atoms defining one or more binding pockets.

In the context of target molecules in this invention, the term “crystal” refers to a regular assemblage of a target molecule of a type suitable for X-ray crystallography. That is, the assemblage produces an X-ray diffraction pattern when illuminated with a beam of X-rays. Thus, a crystal is distinguished from an agglomeration or other complex of target molecule that does not give a diffraction pattern.

By “co-crystal” is meant a complex of the compound, molecular scaffold, or ligand bound non-covalently to the target molecule and present in a crystal form appropriate for analysis by X-ray or protein crystallography. In preferred embodiments the target molecule-ligand complex can be a protein-ligand complex.

The phrase “alter the binding affinity or binding specificity” refers to changing the binding constant of a first compound for another, and/or changing the level of binding of a first compound for a second compound as compared to the level of binding of the first compound for third compounds, respectively. For example, the binding specificity of a compound for a particular protein is increased if the relative level of binding to that particular protein is increased as compared to binding of the compound to unrelated proteins.

As used herein in connection with test compounds, binding compounds, and modulators (ligands), the term “synthesizing” and like terms means chemical synthesis from one or more precursor materials.

The phrase “chemical structure of the molecular scaffold is modified” means that a derivative molecule has a chemical structure that differs from that of the molecular scaffold but still contains common core chemical structural features. The phrase does not necessarily mean that the molecular scaffold is used as a precursor in the synthesis of the derivative.

By “assaying” is meant the creation of experimental conditions and the gathering of data regarding a particular result of the experimental conditions. For example, enzymes can be assayed based on their ability to act upon a detectable substrate. A compound or ligand can be assayed, for example, based on its ability to bind to a particular target molecule or molecules.

Certain compounds have been identified as molecular scaffolds and binding compounds for PYK2. Thus, in another aspect, the invention provides a method for identifying a ligand binding to PYK2, that includes determining whether a derivative compound that includes a core structure of Formula I as described herein binds to PYK2 with altered binding affinity or specificity or both as compared to a parent compound.

In reference to compounds of Formula I, the term “core structure” refers to the ring structure shown diagramatically as part of the description of compounds of Formula I, but excluding substituents. More generally, the term “core structure” refers to a characteristic chemical structure common to a set of compounds, especially a chemical structure than carries variable substituents in the compound set.

By a “set” of compounds is meant a collection of compounds. The compounds may or may not be structurally related.

In another aspect, structural information about PYK2 can also be used to assist in determining a struture for another kinase, e.g., FAK, by creating a homology model from an electronic representation of a PYK2 structure.

Typically creating such a homology model involves identifying conserved amino acid residues between PYK2 and the other kinase of interest; transferring the atomic coordinates of a plurality of conserved amino acids in the PYK2 structure to the corresponding amino acids of the other kinase to provide a rough structure of that kinase; and constructing structures representing the remainder of the other kinase using electronic representations of the structures of the remaining amino acid residues in the other kinase. In particular, coordinates from Table 1 or Table 2 for conserved residues can be used. Conserved residues in a binding site, e.g., PYK2 residues 503, 505, 457, 488, 567, and 554, can be used.

To assist in developing other portions of the kinase structure, the homology model can also utilize, or be fitted with, low resolution X-ray diffraction data from one or more crystals of the kinase, e.g., to assist in linking conserved residues and/or to better specify coordinates for terminal portions of a polypeptide.

The PYK2 structural information used can be for a variety of different PYK2 variants, including full-length wild type, naturally-occurring variants (e.g., allelic variants and splice variants), truncated variants of wild type or naturally-occuring variants, and mutants of full-length or truncated wild-type or naturally-occurring variants (that can be mutated at one or more sites). For example, in order to provide a PYK2 structure closer to a variety of other kinase structures, a mutated PYK2 that includes a mutation to a conserved residue in a binding site can be used (or a plurality of such mutations).

In another aspect, the invention provides a crystalline form of PYK2, which may be a reduced length PYK2 such as a PYK2 kinase domain, e.g., having atomic coordinates as described in Table 1 or Table 2. The crystalline form can contain one or more heavy metal atoms, for example, atoms useful for X-ray crystallography. The crystalline form can also include a binding compound in a co-crystal, e.g., a binding compound that interacts with one more more of PYK2 residues residues 503, 505, 457, 488, 567, and 554 or any two, any three, any four, any five, or all six of those residues, and can, for example, be a compound of Formula I. PYK2 crystals can be in various environments, e.g., in a crystallography plate, mounted for X-ray crystallography, and/or in an X-ray beam. The PYK2 may be of various forms, e.g., a wild-type, variant, truncated, and/or mutated form as described herein.

The invention further concerns co-crystals of PYK2, which may a reduced length PYK2, e.g., a PYK2 kinase domain, and a PYK2 binding compound. Advantageously, such co-crystals are of sufficient size and quality to allow structural determination of PYK2 to at least 3 Angstroms, 2.5 Angstroms, 2.0 Angstroms, or 1.8 Angstroms. The co-crystals can, for example, be in a crystallography plate, be mounted for X-ray crystallography and/or in an X-ray beam. Such co-crystals are beneficial, for example, for obtaining structural information concerning interaction between PYK2 and binding compounds.

PYK2 binding compounds can include compounds that interact with at least one of PYK2 residues 503, 505, 457, 488, 567, and 554, or any 2, 3, 4, 5, or all 6 of those residues. Exemplary compounds that bind to PYK2 include compounds of Formula I.

Likewise, in additional aspects, methods for obtaining PYK2 crystals and co-crystals are provided. In one aspect is provided a method for obtaining a crystal of PYK2 kinase domain, by subjecting PYK2 kinase domain protein at 5-20 mg/ml, preferably 8-12 mg/ml, to crystallization condition as described below, or conditions substantially equivalent thereto:
2-10% (e.g., 8%) polyethylene glycol (PEG) 8000, 0.2 M sodium acetate, 0.1% sodium cacodylate pH 6.5, 20% glycerol.
   In general, the PYK2 will be in a solution containing the protein and suitable buffer. For example, the solution can contain 20 mM Tris-HCl ph 8.0, 150 mM NaCl, 14 mM β-mercaptoethanol (BME), and 1 mM dithiothreitol (DTT).

Crystallization conditions can be initially identified using a screening kit, such as a Hampton Research (Riverside, Calif.) screening kit 1 and/or 2. Conditions resulting in crystals can be selected and crystallization conditions optimized based on the demonstrated crystallization conditions. To assist in subsequent crystallography, the PYK2 can be seleno-methionine labeled. Also, as indicated above, the PYK2 may be any of various forms, e.g., truncated to provide a PYK2 kinase domain, which can be selected to be of various lengths.

In connection with chemical concentrations, the terms “approximately” and “about” mean±20% of the indicated value.

In the context of crystallization conditions, the term “substantially equivalent” means conditions in a range around identified crystallization conditions such that the concentrations of solution components are within ±10% of the stated value, pH is ±1 pH unit, preferable ±0.5 pH unit, polymer, salt, and buffer substitutions may be made so long as one of ordinary skill in the art of protein crystallization would recognize the solution with the substituted component as being likely to also result in crystallization (though re-optimization may be useful). An example of such a substitution can be the substitution of a particular size PEG with a slightly smaller or larger PEG product, or a mixture of both a larger and a smaller PEG product.

A related aspect provides a method for obtaining co-crystals of PYK2, which can be a reduced length PYK2, with a binding compound, by subjecting PYK2 protein at 5-20 mg/ml to crystallization conditions substantially equivalent to the conditions as described above, in the presence of binding compound, for a time sufficient for cystal development. The binding compound may be added at various concentrations depending on the nature of the compound, e.g., final concentration of 0.5 to 1.0 mM. In many cases, the binding compound will be in an organic solvent such as demethyl sulfoxide solution (DMSO). While not preferred, binding compound can also be soaked into a PYK2 crystal, e.g., using conventional techniques.

In another aspect, provision of compounds active on PYK2 also provides a method for modulating PYK2 activity by contacting PYK2 with a compound that binds to PYK2 and interacts with one more of residues residues 503, 505, 457, 488, 567, and 554, for example a compound of Formula I. The compound is preferably provided at a level sufficient to modulate the activity of PYK2 by at least 10%, more preferably at least 20%, 30%, 40%, or 50%. In many embodiments, the compound will be at a concentration of about 1 μM, 100 μM, or 1 mM, or in a range of 1-100 nM, 100-500 nM, 500-1000 nM, 1-100 μM, 100-500 μM, or 500-1000 μM.

As used herein, the term “modulating” or “modulate” refers to an effect of altering a biological activity, especially a biological activity associated with a particular biomolecule such as PYK2. For example, an agonist or antagonist of a particular biomolecule modulates the activity of that biomolecule, e.g., an enzyme.

The term “PYK2 activity” refers to a biological activity of PYK2, particularly including kinase activity.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

In a related aspect, the invention provides a method for treating a patient suffering from or at risk of a disease or condition for which modulation of PYK2 activity provides a therapeutic or prophylactic effect, e.g., a disease or condition characterized by abnormal PYK2 kinase activity, where the method involves administering to the patient a compound that interacts with at least 2, or three or more of PYK2 residues residues 503, 505, 457, 488, 567, and 554 (e.g., a compound of Formula I).

Specific diseases or disorders which might be treated or prevented cells include: myasthenia gravis; neuroblastoma; disorders caused by neuronal toxins such as cholera toxin, pertusis toxin, or snake venom; acute megakaryocytic myelosis; thrombocytopenia; those of the central nervous system such as seizures, stroke, head trauma, spinal cord injury, hypoxia-induced nerve cell damage such as in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disor-der, schizophrenia, neuroleptic malignant syndrome, and Tourette's syndrome. Conditions that may be treated by PYK2 inhibitors include epilepsy, schizophrenia, extreme hyperactivity in children, chronic pain, and acute pain. Examples of conditions that may be treated by PYK2 enhancers (for example a phosphatase inhibitor) include stroke, Alzheimer's, Parkinson's, other neurodegenerative diseases, and migraine.

Preferred disorders include epilepsy, stroke, schizophrenia, and Parkinson's disorder, as there is a well established relationship between these disorders and the function of potassium channels.

In addition, PYK2 can act as a target for therapeutics for treating cell proliferative diseases. Thus, in certain embodiments, the disease or condition is a proliferative disease or neoplasia, such as benign or malignant tumors, psoriasis, leukemias (such as myeloblastic leukemia), lymphoma, prostate cancer, liver cancer, breast cancer, sarcoma, neuroblastima, Wilm's tumor, bladder cancer, thyroid cancer, neoplasias of the epithelialorigin such as mammacarcinoma, a cancer of hematopoietic cells, or a chronic inflammatory disease or condition, resulting, for example, from a persistent infection (e.g., tuberculosis, syphilis, fungal infection), from prolonged exposure to endogenous (e.g., elevated plasma lipids) or exogenous (e.g., silica, asbestos, cigarette tar, surgical sutures) toxins, and from autoimmune reactions (e.g., rheumatoid arthritis, systemic lupus erythrymatosis, multiple sclerosis, psoriasis). Thus, chronic inflammatory diseases include many common medical conditions, such as rheumatoid arthritis, restenosis, psoriasis, multiple sclerosis, surgical adhesions, tuberculosis, and chronic inflammatory lung and airway diseases, such as asthma pheumoconiosis, chronic obstructive pulmonary disease, nasal polyps, and pulmonary fibrosis. PYK2 modulators may also be useful in inhibiting development of hematomous plaque and restinosis, in controlling restinosis, as anti-metastatic agents, in treating diabetic complications, as immunosuppressants, and in control of angiogenesis to the extent a PYK2 kinase is involved in a particular disease or condition.

As crystals of PYK2 have been developed and analyzed, another aspect concerns an electronic representation of PYK2 (which may be a reduced length PYK2), for example, an electronic representation containing atomic coordinate representations corresponding to the coordinates listed for PYK2 in Table 1 or Table 2, or a schematic representation such as one showing secondary structure and/or chain folding, and may also show conserved active site residues. The PYK2 may be wild type, an allelic variant, a mutant form, or a modifed form, e.g., as described herein.

The electronic representation can also be modified by replacing electronic representations of particular residues with electronic representations of other residues. Thus, for example, an electronic representation containing atomic coordinate representations corresponding to the coordinates for PYK2 listed in Table 1 or Table 2 can be modified by the replacement of coordinates for a particular conserved residue in a binding site by a different amino acid. Likewise, a PYK2 representation can be modified by the respective substitutions, insertions, and/or deletions of amino acid residues to provide a representation of a structure for FAK kinase. Following a modification or modifications, the representation of the overall structure can be adjusted to allow for the known interactions that would be affected by the modification or modifications. In most cases, a modification involving more than one residue will be performed in an iterative manner.

In addition, an electronic representation of a PYK2 binding compound or a test compound in the binding site can be included, e.g., a compound of Formula I.

Likewise, in a related aspect, the invention concerns an electronic representation of a portion of a PYK2 kinase, a binding site (which can be an active site) or kinase domain, for example, residues 419-691. A binding site or kinase domain can be represented in various ways, e.g., as representations of atomic coordinates of residues around the binding site and/or as a binding site surface contour, and can include representations of the binding character of particular residues at the binding site, e.g., conserved residues. As for electronic representations of PYK2, a binding compound or test compound may be present in the binding site; the binding site may be of a wild type, variant, mutant form, or modified form of PYK2.

In yet another aspect, the structural information of PYK2 can be used in a homology model (based on PYK2) for another kinase (such as FAK), thus providing an electronic representation of a PYK2 based homology model for a kinase. For example, the homology model can utilize atomic coordinates from Table 1 for conserved amino acid residues. In particular embodiments; atomic coordinates for a wild type, variant, modified form, or mutated form of PYK2 can be used, including, for example, wild type, variants, modified forms, and mutant forms as described herein. In particular, PYK2 structure provides a very close homology model for FAK kinases. Thus, in particular embodiments the invention provides PYK2-based homology models of FAK.

In still another aspect, the invention provides an electronic representation of a modified PYK2 crystal structure, that includes an electronic representation of the atomic coordinates of a modified PYK2. In an exemplary embodiment, atomic coordinates of Table 1 or Table 2 can be modified by the replacement of atomic coordinates for a particular amino acid with atomic coordinates for a different amino acid. Modifications can include substitutions, deletions (e.g., C-terminal and/or N-terminal detections), insertions (internal, C-terminal, and/or N-terminal) and/or side chain modifications.

In another aspect, the PYK2 structural information provides a method for developing useful biological agents based on PYK2, by analyzing a PYK2 structure to identify at least one sub-structure for forming the biological agent. Such sub-structures can include epitopes for antibody formation, and the method includes developing antibodies against the epitopes, e.g., by injecting an epitope presenting composition in a mammal such as a rabbit, guinea pig, pig, goat, or horse. The sub-structure can also include a mutation site at which mutation is expected to or is known to alter the activity of the PYK2, and the method includes creating a mutation at that site. Still further, the sub-structure can include an attachment point for attaching a separate moiety, for example, a peptide, a polypeptide, a solid phase material (e.g., beads, gels, chromatographic media, slides, chips, plates, and well surfaces), a linker, and a label (e.g., a direct label such as a fluorophore or an indirect label, such as biotin or other member of a specific binding pair). The method can include attaching the separate moiety.

In another aspect, the invention provides a method for identifying potential PYK2, binding compounds by fitting at least one electronic representation of a compound in an electronic representation of a PYK2 binding site. The representation of the binding site may be part of an electronic representation of a larger portion(s) or all of a PYK2 molecule or may be a representation of only the binding site or active site. The electronic representation may be as described above or otherwise described herein.

In particular embodiments, the method involves fitting a computer representation of a compound from a computer database with a computer representation of the active site of a PYK2 kinase, and involves removing a computer representation of a compound complexed with the PYK2 molecule and identifying compounds that best fit the active site based on favorable geometric fit and energetically favorable complementary interactions as potential binding compounds.

In other embodiments, the method involves modifying a computer representation of a compound complexed with a PYK2 molecule, by the deletion or addition or both of one or more chemical groups; fitting a computer representation of a compound from a computer database with a computer representation of the active site of the PYK2 molecule; and identifying compounds that best fit the active site based on favorable geometric fit and energetically favorable complementary interactions as potential binding compounds.

In still other embodiments, the method involves removing a computer representation of a compound complexed with a PYK2 kinase, and searching a database for compounds having structural similarity to the complexed compound using a compound searching computer program or replacing portions of the complexed compound with similar chemical structures using a compound construction computer program.

Fitting a compound can include determining whether a compound will interact with one or more of PYK2 residues residues 503, 505, 457, 488, 567, and 554. Compounds selected for fitting or that are complexed with PYK2 can, for example, be compounds of Formula I.

In another aspect, the invention concerns a method for attaching a PYK2 kinase binding compound to an attachment component, as well as a method for indentifying attachment sites on a PYK2 kinase binding compound. The method involves identifying energetically allowed sites for attachment of an attachment component for the binding compound bound to a binding site of PYK2; and attaching the compound or a derivative thereof to the attachment component at the energetically allowed site.

As used in connection with binding compounds, an “attachment component” refers to a moiety that is attached to a binding compound for adding a functionality other than binding with the target molecule and that does not prevent such binding. Examples include direct and indirect labels, linkers, and hapten and other specific recognition moieties. Linkers (including traceless linkers) can be incorporated, for example, for attachment to a solid phase or to another molecule or other moiety. Such attachment can be formed by synthesizing the compound or derivative on the linker attached to a solid phase medium e.g., in a combinatorial synthesis in a plurality of compound. Likewise, the attachment to a solid phase medium can provide an affinity medium (e.g., for affinity chromatography). Labels can be a directly detectable label such as a fluorophore, or an indirectly detectable such as a member of a specific binding pair, e.g., biotin.

The ability to identify energentically allowed sites on a PYK2 kinase binding compound also, in a related aspect, provides modified binding compounds that have linkers attached, for example, compounds of Formula I, preferably at an energetically allowed site for binding of the modified compound to PYK2. The linker can be attached to an attachment component as described above.

Another aspect concerns a modified PYK2 polypeptide that includes a modification that makes the modified PYK2 more similar than native PYK2 to another kinase, and can also include other mutations or other modifications. In various embodiments, the polypeptide includes a full-length PYK2 polypeptide, includes a modified PYK2 binding site, includes at least 20, 30, 40, 50, 60, 70, or 80 contiguous amino acid residues derived from PYK2 including a conserved site.

Still another aspect of the invention concerns a method for developing a ligand for a kinase that includes conserved residues matching any one, 2, 3, 4, 5, or 6 of PYK2 residues 503, 505, 457, 488, 567, and 554, by determining whether a compound of Formula I binds to the kinase. The method can also include determining whether the compound modulates the activity of the kinase. Preferably the kinase has at least 50, 55, 60, or 70% identity over an equal length kinase domain segment.

In particular embodiments, the determining includes computer fitting the compound in a binding site of the kinase and/or the method includes forming a co-crystal of the kinase and the compound. Such co-crystals can be used for determing the binding orientation of the compound with the kinase and/or provide structural information on the kinase, e.g., on the binding site and interacting amino acid residues. Such binding orientation and/or other structural information can be accomplished using X-ray crystallography.

Reference to “matching” of a specified conserved amino acid residue in a kinase domain means that in a maximal alignment of the amino acid sequences of that kinase domain with a different kinase domain, there is an amino acid residue aligned with the specified residue that is either the same amino acid or represents a conservative substitution. Preferably, the matching amino acid residue is within 5 angstroms rms in an overlay of crystal structure atomic coordinates for backbone atoms.

The invention also provides compounds that bind to and/or modulate (e.g., inhibit) PYK2, e.g., PYK2 kinase activity. Accordingly, in aspects and embodiments involving PYK2 binding compounds, molecular scaffolds, and ligands or modulators, the compound is a weak binding compound; a moderate binding compound; a strong binding compound; the compound interacts with one or more of PYK2 residues 503, 505, 457, 488, 567, and 554; the compound is a small molecule; the compound binds to a plurality of different kinases (e.g., at least 3, 5, 10, 15, 20 different kinases). In particular embodiments, the invention concerns compounds of Formula I, as described below.

Thus, in certain embodiments, the invention concerns compounds of Formula I: 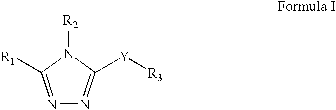
where:
R¹ is hydrogen, trifluormethyl, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, or NR¹⁶R¹⁷;
R² is hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, C(X)NR¹⁶R¹⁷, or —S(O₂)R²¹;
R³ is hydrogen, trifluoromethyl, optionally substituted alkoxyl, optionally substituted thioalkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;
R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl;
R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;
R²¹ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;
X=O, or S.
Y=S, O, NR¹⁶R¹⁷, —C(X)R²⁰, or optionally substituted alkyl.

In Formula I and the descriptions of substituents, subscripts and superscripts are to be regarded as equivalent.

In certain embodiments involving compounds of Formula I, X and Y are 0; X is O and Y is S; X is O and Y is NR¹⁶R¹⁷; X is O and Y is —C(X)R²⁰; X is S and Y is O; X is S and Y is S; X is S and Y is and Y is NR¹⁶R¹⁷; X is S and Y is —C(X)R²⁰.

In certain embodiments, X=O, Y=O, and R¹ is hydrogen; X=O, Y=O, and R² is hydrogen; X=O, Y=S, and R¹ is hydrogen; X=O, Y=S, and R is hydrogen; X=O, Y=NR¹⁶R¹⁷, and R¹ is hydrogen; X=O, Y=S, and R² is hydrogen; X=O, Y=N R¹⁶R¹⁷, and R² is hydrogen; X=O, Y=—C(X)R²⁰, and R¹ is hydrogen; X=O, Y=—C(X)R²⁰, and R² is hydrogen; X=O, Y=optionally substituted alkyl, and R¹ is hydrogen; X=O, Y=optionally substituted alkyl, and R² is hydrogen.

In certain embodiments, X=S, Y=O, and R¹ is hydrogen; X=S, Y=O, and R² is hydrogen; X=S, Y=S, and R¹ is hydrogen; X=S, Y=S, and R² is hydrogen; X=S, Y=NR¹⁶R¹⁷, and R¹ is hydrogen; X=S, Y=S. and R² is hydrogen; X=S, Y=N R=⁶R⁷, and R² is hydrogen; X=S, Y=C(X)R²⁰, and R¹ is hydrogen; X=S, Y=—C(X)R²⁰, and R² is hydrogen; X=S. Y=optionally substituted alkyl, and R¹ is hydrogen; X=S. Y=optionally substituted alkyl, and R¹ is hydrogen.

In certain embodiments, R¹ is hydrogen, optionally substituted lower alkyl, optionally substituted cycloalkyl, or NR¹⁶R¹⁷.

In certain embodiments, R² is hydrogen, optionally substituted lower alkyl, optionally substituted cycloalkyl, C(X)NR¹⁶R¹⁷, or —S(O₂)R²¹.

An additional aspect of this invention relates to pharmaceutical formulations, that include a therapeutically effective amount of a compound of Formula I and at least one pharmaceutically acceptable carrier or excipient. The composition can include a plurality of different pharmacalogically active compounds.

“Halo” or “Halogen”—alone or in combination means all halogens, that is, chloro (Cl), fluoro (F), bromo (Br), iodo (I).

“Hydroxyl” refers to the group —OH.

“Thiol” or “mercapto” refers to the group —SH.

“Alkyl”—alone or in combination means an alkane-derived radical containing from 1 to 20, preferably 1 to 15, carbon atoms (unless specifically defined). It is a straight chain alkyl, branched alkyl or cycloalkyl. Preferably, straight or branched alkyl groups containing from 1-15, more preferably 1 to 8, even more preferably 1-6, yet more preferably 1-4 and most preferably 1-2, carbon atoms, such as methyl, ethyl, propyl, isopropyl, butyl, t-butyl and the like. The term “lower alkyl” is used herein to describe the straight chain alkyl groups described immediately above. Preferably, cycloalkyl groups are monocyclic, bicyclic or tricyclic ring systems of 3-8, more preferably 3-6, ring members per ring, such as cyclopropyl, cyclopentyl, cyclohexyl, adamantyl and the like. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl. A substituted alkyl is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Alkenyl”—alone or in combination means a straight, branched, or cyclic hydrocarbon containing 2-20, preferably 2-17, more preferably 2-10, even more preferably 2-8, most preferably 2-4, carbon atoms and at least one, preferably 1-3, more preferably 1-2, most preferably one, carbon to carbon double bond. In the case of a cycloalkyl group, conjugation of more than one carbon to carbon double bond is not such as to confer aromaticity to the ring. Carbon to carbon double bonds may be either contained within a cycloalkyl portion, with the exception of cyclopropyl, or within a straight chain or branched portion. Examples of alkenyl groups include ethenyl, propenyl, isopropenyl, butenyl, cyclohexenyl, cyclohexenylalkyl and the like. A substituted alkenyl is the straight chain alkenyl, branched alkenyl or cycloalkenyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, carboxy, alkoxycarbonyl, aryloxycarbonyl, heteroaryloxycarbonyl, or the like attached at any available point to produce a stable compound.

“Alkynyl”—alone or in combination means a straight or branched hydrocarbon containing 2-20, preferably 2-17, more preferably 2-10, even more preferably 2-8, most preferably 2-4, carbon atoms containing at least one, preferably one, carbon to carbon triple bond. Examples of alkynyl groups include ethynyl, propynyl, butynyl and the like. A substituted alkynyl refers to the straight chain alkynyl or branched alkenyl defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like attached at any available point to produce a stable compound.

“Alkyl alkenyl” refers to a group —R—CR′═CR′″ R″″, where R is lower alkyl, or substituted lower alkyl, R′, R′″, R″″ may independently be hydrogen, halogen, lower alkyl, substituted lower alkyl, acyl, aryl, substituted aryl, hetaryl, or substituted hetaryl as defined below.

“Alkyl alkynyl” refers to a groups —RCCR′ where R is lower alkyl or substituted lower alkyl, R′ is hydrogen, lower alkyl, substituted lower alkyl, acyl, aryl, substituted aryl, hetaryl, or substituted hetaryl as defined below.

“Alkoxy” denotes the group —OR, where R is lower alkyl, substituted lower alkyl, acyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

“Alkylthio” or “thioalkoxy” denotes the group —SR, —S(O)ₙ₌₁₋₂—R, where R is lower alkyl, substituted lower alkyl, aryl, substituted aryl, aralkyl or substituted aralkyl as defined herein.

“Acyl” denotes groups —C(O)R, where R is hydrogen, lower alkyl substituted lower alkyl, aryl, substituted aryl and the like as defined herein.

“Aryloxy” denotes groups —OAr, where Ar is an aryl, substituted aryl, heteroaryl, or substituted heteroaryl group as defined herein.

“Amino” or substituted amine denotes the group NRR′, where R and R′ may independently by hydrogen, lower alkyl, substituted lower alkyl, aryl, substituted aryl, hetaryl, or substituted heteroaryl as defined herein, acyl or sulfonyl.

“Amido” denotes the group —C(O)NRR′, where R and R′ may independently by hydrogen, lower alkyl, substituted lower alkyl, aryl, substituted aryl, hetaryl, substituted hetaryl as defined herein.

“Carboxyl” denotes the group —C(O)OR, where R is hydrogen, lower alkyl, substituted lower alkyl, aryl, substituted aryl, hetaryl, and substituted hetaryl as defined herein.

“Aryl”—alone or in combination means phenyl or naphthyl optionally carbocyclic fused with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Substituted aryl” refers to aryl optionally substituted with one or more functional groups, e.g., halogen, lower alkyl, lower alkoxy, alkylthio, acetylene, amino, amido, carboxyl, hydroxyl, aryl, aryloxy, heterocycle, heteroaryl, substituted heteroaryl, nitro, cyano, thiol, sulfamido and the like.

“Heterocycle” refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl or benzo[b]thienyl) and having at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heteroaryl”—alone or in combination means a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group O, S, and N, and optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. Examples of heteroaryl groups are pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl and the like. A substituted heteroaryl contains a substituent attached at an available carbon or nitrogen to produce a stable compound.

“Heterocyclyl”—alone or in combination means a non-aromatic cycloalkyl group having from 5 to 10 atoms in which from 1 to 3 carbon atoms in the ring are replaced by heteroatoms of O, S or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. Examples of heterocyclyl groups are tetrahydrofuranyl, dihydropyridinyl, piperidinyl, pyrrolidinyl, piperazinyl, dihydrobenzofuryl, dihydroindolyl, and the like. A substituted hetercyclyl contains a substituent nitrogen attached at an available carbon or nitrogen to produce a stable compound.

“Substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Aralkyl” refers to the group —R—Ar where Ar is an aryl group and R is lower alkyl or substituted lower alkyl group. Aryl groups can optionally be unsubstituted or substituted with, e.g., halogen, lower alkyl, alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Heteroalkyl” refers to the group -R-Het where Het is a heterocycle group and R is a lower alkyl group. Heteroalkyl groups can optionally be unsubstituted or substituted with e.g., halogen, lower alkyl, lower alkoxy, alkylthio, acetylene, amino, amido, carboxyl, aryl, aryloxy, heterocycle, substituted heterocycle, hetaryl, substituted hetaryl, nitro, cyano, thiol, sulfamido and the like.

“Heteroarylalkyl” refers to the group -R-HetAr where HetAr is an heteroaryl group and R lower alkyl or substituted lower alkyl. Heteroarylalkyl groups can optionally be unsubstituted or substituted with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloalkyl” refers to a divalent cyclic or polycyclic alkyl group containing 3 to 15 carbon atoms.

“Substituted cycloalkyl” refers to a cycloalkyl group comprising one or more substituents with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloheteroalkyl” refers to a cycloalkyl group wherein one or more of the ring carbon atoms is replaced with a heteroatom (e.g., N, O, S or P).

“Substituted cycloheteroalkyl” refers to a cycloheteroalkyl group as herein defined which contains one or more substituents, such as halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloalkyl” denotes the group —R-cycloalkyl where cycloalkyl is a cycloalkyl group and R is a lower alkyl or substituted lower alkyl. Cycloalkyl groups can optionally be unsubstituted or substituted with e.g. halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloheteroalkyl” denotes the group -R-cycloheteroalkyl where R is a lower alkyl or substituted lower alkyl. Cycloheteroalkyl groups can optionally be unsubstituted or substituted with e.g. halogen, lower alkyl, lower alkoxy, alkylthio, amino, amido, carboxyl, acetylene, hydroxyl, aryl, aryloxy, heterocycle, substituted heterocycle, hetaryl, substituted hetaryl, nitro, cyano, thiol, sulfamido and the like.

In addition to compounds (including molecular scaffolds) of Formula I as described herein, additional types of compounds can be used as modulators (e.g., inhibitors) of PYK2, and for development of further PYK2 ligands. In particular, compounds of the types described in Bremer et al., U.S. application Ser. No. 10/664,421, filed Sep. 16, 2003, and Bremer et al., U.S. Application 60/503,277, filed Sep. 15, 2003, both of which are incorporated herein in their entireties, including drawings.

An additional aspect of this invention relates to pharmaceutical formulations, that include a therapeutically effective amount of a compound of Formula I, and at least one pharmaceutically acceptable carrier or excipient. The composition can include a plurality of different pharmacalogically active compounds.

Additional aspects and embodiments will be apparent from the following Detailed Description and from the claims.

### BRIEF DESCRIPTION OF THE DRAWINGS

FIG. 1 shows a ribbon diagram schematic representation of PYK2 active site.

### DETAILED DESCRIPTION OF THE PREFERRED EMBODIMENTS

The Tables will first be briefly described.

Table 1 provides atomic coordinates for human PYK2 kinase domain. In this table and in Table 2, the various columns in the lines beginning with “ATOM” have the following content, beginning with the left-most column:
ATOM: Refers to the relevant moiety for the table row.
Atom number: Refers to the arbitrary atom number designation within the coordinate table.
Atom Name: Identifier for the atom present at the particular coordinates.
Chain ID: Chain ID refers to one monomer of the protein in the crystal, e.g., chain “A”, or to other compound present in the crystal, e.g., HOH for water, and L for a ligand or binding compound. Multiple copies of the protein monomers will have different chain Ids.
Residue Number: The amino acid residue number in the chain.
X, Y, Z: Respectively are the X, Y, and Z coordinate values.
Occupancy: Describes the fraction of time the atom is observed in the crystal. For example, occupancy=1 means that the atom is present all the time; occupancy=0.5 indicates that the atom is present in the location 50% of the time.
B-factor: A measure of the thermal motion of the atom.
Element: Identifier for the element.

In addition, the lines that begin with “ANISOU” present the anisotropic temperature factors. The anisotropic temperture factors are related to the corresponding isotropic temperature factors (B-factors) in the “ATOM” lines in the table. Following “ANISOU”, the next 4 entries are “Atom number”, “Atom name”, Residue name”, and “Residue number”, and are the same as the respective corresponding “ATOM” line entries. The next 6 entries are the anisotropic temperature factors U(1,1 l), U(2,2), U(3,3), U1,2), U(1,3), and U(2,3) in order (scaled by a factor of 10⁴ (Angstroms²) and presented as integers).

Table 2 provides atomic coordinates for PYK2 with (5′-adenylylimidodiphosphate) AMPPNP in the binding site.

Table 3 provides an alignment of kinase domains for several kinases, including human PYK2, providing identification of residues conserved between various members of the set. The residue number is for PYK2.

Table 4 provides the nucleic acid and amino acid sequences for human PYK2 kinase domain.

Table 5 provides representative assay results for kinase activity of PYK2 kinase domain in the presence of ATP and in the presence of several ATP analogs.

I. Introduction

The present invention concerns the use of PYK2 kinase structures, structural information, and related compositions for identifying compounds that modulate PYK2 kinase activity and for determining structuctures of other kinases.

PYK2 kinase is involved in a number of disease conditions. For example, as indicated in the Background above, PYK2 functions as a neurotransmitter regulator, and thus modulation of PYK2 can enhance or inhibit such signaling. In addition, due to the involve ment of PYK2 in linking the G protein-coupled pathway with the sos/grb pathway for MAP kinase signal tranduction activation. This may involve the binding of src. Thus, PYK2 can also affect cell proliferation.

Exemplary Diseases Associated with PYK2.

As indicated above, modulation of PYK2 activity is beneficial for treatment or prevention of a variety of diseases and conditions, such as those relating to its roles in signal transduction. As a result, PYK2 inhibitors have therapeutic applications in the treatment of proliferative diseases, such as various cancers, osteoporosis, and inflammation, as well as other disease states, such as those referenced in the Summary above and those otherwise indicated herein. PYK2, sceening for PYK2 modulators, and methods for using PYK2 modulators, along with related assays, techniques, and data, are described, for example, in Duong et al., PCT Application No. PCT/US98/02792, PCT Publication WO/98/35056; Schlessinger et al., PCT Application No. PCT/US98/27871, PCT Publication WO 00/40971; Lev, et al., PCT Application PCT/US97/22565, PCT Publication WO 98/26054; Lev et al., PCT Application PCT/US95/15846, PCT Publication WO 96/18738, which are incorporated herein in their entireties.

Osteoporosis

Activation of osteoclasts is initiated by adhesion of osteoclast to bone surface. Cytoskeletal rearrangement results in formation of a sealing zone and a polarized ruffled membrane. Pyk2 was found to be highly expressed in osteoclasts. (Duong et al. (1998) “Pyk2 in osteoclasts is an adhesion kinase, localized in the sealing zone, activated by ligation of alpha(v)beta3 integrin, and phosphorylated by Src kinase.” J. Clin. Invest. 102:881-892.) Studies indicate that Pyk2 is involved in the adhesion-induced formation of the sealing zone and is required for osteoclast bone resorption. (Duong and Rodan (1998) Integrin-mediated signaling in the regulation of osteoclast adhesion and activation.” Front. Biosci. 3:757-768.)

Proliferative Diseases

In another example, modulation of PYK2 has been indicated for treatment of proliferative diseases such as cancer, e.g., for cancers of hematopoietic cells, among others. (Avraham et al., PCT Publication 98/07870, which is incorporated herein by reference in its entirety.)

Inflammation

Modulation of PYK2 has also been linked with treatment of inflammatory response-related diseases, generally those that have an aberrent inflammatory response, for example, inflammatory bowel diseases such as ulcerative colitis and Crohn's Disease, and connective tissue diseases such as rheumatoid arthritis, system lupus erythrmatosus, progressive systemin sclerosis, mixed connective tissue disease, and Sjogren's syndrome. (Schlessinger et al., PCT Publication WO 00/40971, which is incorporated herein by reference in its entirety.) A pathologic inflammatory response may be a continuation of an acute inflammatory response, or a prolonged low-grade inflammatory response, and typically results in tissue damage. Macrophage and T-cell recruitment, and process such as cytokine production can directly contribute to inflammatory pathogenesis.

II. Crystalline PYK2 Kinase

Crystalline PYK2 kinases (e.g., human PYK2) include native crystals, kinase domain crystals, derivative crystals, and co-crystals. The crystals generally comprise substantially pure polypeptides corresponding to the PYK2 kinase polyeptide in crystalline form. In connection with the development of inhibitors of PYK2 kinase function, it is advantageous to use PYK2 kinase domain for structural determination, because use of the reduced sequence simplifies structure determination. To be useful for this purpose, the kinase domain should be active and/or retain native-type binding, thus indicating that the kinase domain takes on substantially normal 3D structure.

It is to be understood that the crystalline kinases and kinase domains useful in the the invention are not limited to naturally occurring or native kinase. Indeed, the crystals include crystals of mutants of native kinases. Mutants of native kinases are obtained by replacing at least one amino acid residue in a native kinase with a different amino acid residue, or by adding or deleting amino acid residues within the native polypeptide or at the N- or C-terminus of the native polypeptide, and have substantially the same three-dimensional structure as the native kinase from which the mutant is derived.

By having substantially the same three-dimensional structure is meant having a set of atomic structure coordinates that have a root-mean-square deviation of less than or equal to about 2 Å when superimposed with the atomic structure coordinates of the native kinase from which the mutant is derived when at least about 50% to 100% of the Ca atoms of the native kinase or kinase domain are included in the superposition.

Amino acid substitutions, deletions and additions which do not significantly interfere with the three-dimensional structure of the kinase will depend, in part, on the region of the kinase where the substitution, addition or deletion occurs. In highly variable regions of the molecule, non-conservative substitutions as well as conservative substitutions may be tolerated without significantly disrupting the three-dimensional, structure of the molecule. In highly conserved regions, or regions containing significant secondary structure, conservative amino acid substitutions are preferred. Such conserved and variable regions can be identified by sequence alignment of PYK2 with other kinases. Such alignment of PYK2 kinase domain along with a number of other kinase domains is provided in Table 3.

Conservative amino acid substitutions are well known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

For kinases obtained in whole or in part by chemical synthesis, the selection of amino acids available for substitution or addition is not limited to the genetically encoded amino acids. Indeed, the mutants described herein may contain non-genetically encoded amino acids. Conservative amino acid substitutions for many of the commonly known non-genetically encoded amino acids are well known in the art. Conservative substitutions for other amino acids can be determined based on their physical properties as compared to the properties of the genetically encoded amino acids.

In some instances, it may be particularly advantageous or convenient to substitute, delete and/or add amino acid residues to a native kinase in order to provide convenient cloning sites in cDNA encoding the polypeptide, to aid in purification of the polypeptide, and for crystallization of the polypeptide. Such substitutions, deletions and/or additions which do not substantially alter the three dimensional structure of the native kinase domain will be apparent to those of ordinary skill in the art.

It should be noted that the mutants contemplated herein need not all exhibit kinase activity. Indeed, amino acid substitutions, additions or deletions that interfere with the kinase activity but which do not significantly alter the three-dimensional structure of the domain are specifically contemplated by the invention. Such crystalline polypeptides, or the atomic structure coordinates obtained therefrom, can be used to identify compounds that bind to the native domain. These compounds can affect the activity of the native domain.

The derivative crystals of the invention can comprise a crystalline kinase polypeptide in covalent association with one or more heavy metal atoms. The polypeptide may correspond to a native or a mutated kinase. Heavy metal atoms useful for providing derivative crystals include, by way of example and not limitation, gold, mercury, selenium, etc.

The co-crystals of the invention generally comprise a crystalline kinase domain polypeptide in association with one or more compounds. The association may be covalent or non-covalent. Such compounds include, but are not limited to, cofactors, substrates, substrate analogues, inhibitors, allosteric effectors, etc.

Exemplary mutations for PYK2 family kinases include the insertion of a sequence having the FAK sequence shown in the FIG. 3 alignment between PYK2 residues 482 and 483. Such insertion is useful, for example, to assist in using PYK2 kinases to model FAK kinase. Mutations at other sites can likewise be carried out, e.g., to make a mutated PYK2 kinase more similar to another kinase for structure modeling and/or compound fitting purposes, such as a kinase in the kinase domain alignment in Table 3.

III. Three Dimensional Structure Determination Using X-ray Crystallography

X-ray crystallography is a method of solving the three dimensional structures of molecules. The structure of a molecule is calculated from X-ray diffraction patterns using a crystal as a diffraction grating. Three dimensional structures of protein molecules arise from crystals grown from a concentrated aqueous solution of that protein. The process of X-ray crystallography can include the following steps:
(a) synthesizing and isolating (or otherwise obtaining) a polypeptide;
(b) growing a crystal from an aqueous solution comprising the polypeptide with or without a modulator; and
(c) collecting X-ray diffraction patterns from the crystals, determining unit cell dimensions and symmetry, determining electron density, fitting the amino acid sequence of the polypeptide to the electron density, and refining the structure.

Production of Polypeptides

The native and mutated kinase polypeptides described herein may be chemically synthesized in whole or part using techniques that are well-known in the art (see, e.g., Creighton (1983) Biopolymers 22(1):49-58).

Alternatively, methods which are well known to those skilled in the art can be used to construct expression vectors containing the native or mutated kinase polypeptide coding sequence and appropriate transcriptional/translational control signals. These methods include in vitro recombinant DNA techniques, synthetic techniques and in vivo recombination/genetic recombination. See, for example, the techniques described in Maniatis, T (1989). Molecular cloning: A laboratory Manual. Cold Spring Harbor Laboratory, N.Y. Cold Spring Harbor Laboratory Press; and Ausubel, F. M. et al. (1994) Current Protocols in Molecular Biology. John Wiley & Sons, Secaucus, N.J.

A variety of host-expression vector systems may be utilized to express the kinase coding sequence. These include but are not limited to microorganisms such as bacteria transformed with recombinant bacteriophage DNA, plasmid DNA or cosmid DNA expression vectors containing the kinase domain coding sequence; yeast transformed with recombinant yeast expression vectors containing the kinase domain coding sequence; insect cell systems infected with recombinant virus expression vectors (e.g., baculovirus) containing the kinase domain coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the kinase domain coding sequence; or animal cell systems. The expression elements of these systems vary in their strength and specificities.

Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genorne of mammalian cells (e.g., metallothionein promoter) or from mammalian viruses (e.g., the adenovirus late promoter; the vaccinia virus 7.5K promoter) may be used; when generating cell lines that contain multiple copies of the kinase domain DNA, SV4O-, BPV- and EBV-based vectors may be used with an appropriate selectable marker.

Exemplary methods describing methods of DNA manipulation, vectors, various types of cells used, methods of incorporating the vectors into the cells, expression techniques, protein purification and isolation methods, and protein concentration methods are disclosed in detail in PCT publication WO 96/18738. This publication is incorporated herein by reference in its entirety, including any drawings. Those skilled in the art will appreciate that such descriptions are applicable to the present invention and can be easily adapted to it.

Crystal Growth

Crystals are grown from an aqueous solution containing the purified and concentrated polypeptide by a variety of techniques. These techniques include batch, liquid, bridge, dialysis, vapor diffusion, and hanging and sitting drop methods. McPherson (1982) John Wiley, New York; McPherson (1990) Eur. J. Biochem. 189:1-23; Webber (1991) Adv. Protein Chem. 41:1-36, incorporated by reference herein in their entireties, including all figures, tables, and drawings.

The native crystals of the invention are, in general, grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

For crystals of the invention, exemplary crystallization conditions are described in the Examples. Those of ordinary skill in the art will recognize that the exemplary crystallization conditions can be varied. Such variations may be used alone or in combination. In addition, other crystallization conditions may be found, e.g., by using crystallization screening plates to identify such other conditions. Those alternate conditions can then be optimized if needed to provide larger or better quality crystals.

Derivative crystals of the invention can be obtained by soaking native crystals in mother liquor containing salts of heavy metal atoms. Exemplary conditions for such soaking a native crystal utilizes a solution containing about 0.1 mM to about 5 mM thimerosal, 4-chloromeruribenzoic acid or KAu(CN)₂ for about 2 hr to about 72 hr to provide derivative crystals suitable for use as isomorphous replacements in determining the X-ray crystal structure.

Co-crystals of the invention can be obtained by soaking a native crystal in mother liquor containing compound that binds the kinase, or can be obtained by co-crystallizing the kinase polypeptide in the presence of a binding compound.

In many cases, co-crystallization of kinase and binding compound can be accomplished using conditions identified for crystallizing the corresponding kinase without binding compound. It is advantageous if a plurality of different crystallization conditions have been identified for the kinase, and these can be tested to determine which condition gives the best co-crystals. It may also be benficial to optimize the conditions for co-crystallization. Alternatively, new crystallization conditions can be determined for obtaining co-crystals, e.g., by screening for crystallization and then optimizing those conditions. Exemplary co-crystallization conditions are provided in the Examples.

Determining Unit Cell Dimensions and the Three Dimensional Structure of a Polypeptide or Polypeptide Complex

Once the crystal is grown, it can be placed in a glass capillary tube or other mounting device and mounted onto a holding device connected to an X-ray generator and an X-ray detection device. Collection of X-ray diffraction patterns are well documented by those in the art. See, e.g., Ducruix and Geige, (1992), IRL Press, Oxford, England, and references cited therein. A beam of X-rays enters the crystal and then diffracts from the crystal. An X-ray detection device can be utilized to record the diffraction patterns emanating from the crystal. Although the X-ray detection device on older models of these instruments is a piece of film, modern instruments digitally record X-ray diffraction scattering. X-ray sources can be of various types, but advantageously, a high intensity source is used, e.g., a synchrotron beam source.

Methods for obtaining the three dimensional structure of the crystalline form of a peptide molecule or molecule complex are well known in the art. See, e.g., Ducruix and Geige, (1992), IRL Press, Oxford, England, and references cited therein. The following are steps in the process of determining the three dimensional structure of a molecule or complex from X-ray diffraction data.

After the X-ray diffraction patterns are collected from the crystal, the unit cell dimensions and orientation in the crystal can be determined. They can be determined from the spacing between the diffraction emissions as well as the patterns made from these emissions. The unit cell dimensions are characterized in three dimensions in units of Angstroms (one Å=10⁻¹⁰ meters) and by angles at each vertices. The symmetry of the unit cell in the crystals is also characterized at this stage. The symmetry of the unit cell in the crystal simplifies the complexity of the collected data by identifying repeating patterns. Application of the symmetry and dimensions of the unit cell is described below.

Each diffraction pattern emission is characterized as a vector and the data collected at this stage of the method determines the amplitude of each vector. The phases of the vectors can be determined using multiple techniques. In one method, heavy atoms can be soaked into a crystal, a method called isomorphous replacement, and the phases of the vectors can be determined by using these heavy atoms as reference points in the X-ray analysis. (Otwinowski, (1991), Daresbury, United Kingdom, 80-86). The isomorphous replacement method usually utilizes more than one heavy atom derivative.

In another method, the amplitudes and phases of vectors from a crystalline polypeptide with an already determined structure can be applied to the amplitudes of the vectors from a crystalline polypeptide of unknown structure and consequently determine the phases of these vectors. This second method is known as molecular replacement and the protein structure which is used as a reference should have a closely related structure to the protein of interest. (Naraza (1994) Proteins 11:281-296). Thus, the vector information from a kinase of known structure, such as those reported herein, are useful for the molecular replacement analysis of another kinase with unknown structure.

Once the phases of the vectors describing the unit cell of a crystal are determined, the vector amplitudes and phases, unit cell dimensions, and unit cell symmetry can be used as terms in a Fourier transform function. The Fourier transform function calculates the electron density in the unit cell from these measurements. The electron density that describes one of the molecules or one of the molecule complexes in the unit cell can be referred to as an electron density map. The amino acid structures of the sequence or the molecular structures of compounds complexed with the crystalline polypeptide may then be fitted to the electron density using a variety of computer programs. This step of the process is sometimes referred to as model building and can be accomplished by using computer programs such as Turbo/FRODO or “O”. (Jones (1985) Methods in Enzymology 115:157-171).

A theoretical electron density map can then be calculated from the amino acid structures fit to the experimentally determined electron density. The theoretical and experimental electron density maps can be compared to one another and the agreement between these two maps can be described by a parameter called an R-factor. A low value for an R-factor describes a high degree of overlapping electron density between a theoretical and experimental electron density map.

The R-factor is then minimized by using computer programs that refine the theoretical electron density map. A computer program such as X-PLOR can be used for model refinement by those skilled in the art. (Brünger (1992) Nature 355:472-475.) Refinement may be achieved in an iterative process. A first step can entail altering the conformation of atoms defined in an electron density map. The conformations of the atoms can be altered by simulating a rise in temperature, which will increase the vibrational frequency of the bonds and modify positions of atoms in the structure. At a particular point in the atomic perturbation process, a force field, which typically defines interactions between atoms in terms of allowed bond angles and bond lengths, Van der Waals interactions, hydrogen bonds, ionic interactions, and hydrophobic interactions, can be applied to the system of atoms. Favorable interactions may be described in terms of free energy and the atoms can be moved over many iterations until a free energy minimum is achieved. The refinement process can be iterated until the R-factor reaches a minimum value.

The three dimensional structure of the molecule or molecule complex is described by atoms that fit the theoretical electron density characterized by a minimum R-value. A file can then be created for the three dimensional structure that defines each atom by coordinates in three dimensions. An example of such a structural coordinate file is shown in Table 1.

IV. Structures of PYK2

The present invention provides high-resolution three-dimensional structures and atomic structure coordinates of crystalline PYK2 kinase domain and PYK2 kinase domain co-complexed with exemplary binding compounds as determined by X-ray crystallography. The methods used to obtain the structure coordinates are provided in the examples. The atomic structure coordinates of crystalline PYK2 are listed in Table 1, and atomic coordinates for PYK2 co-crystallized with AMPPNP are provided in Table 2. Co-crystal coordinates can be used in the same way, e.g., in the various aspects described herein, as coordinates for the protein by itself.

Those having skill in the art will recognize that atomic structure coordinates as determined by X-ray crystallography are not without error. Thus, it is to be understood that any set of structure coordinates obtained for crystals of PYK2, whether native crystals, kinase domain crystals, derivative crystals or co-crystals, that have a root mean square deviation (“r.m.s.d.”) of less than or equal to about 1.5 Å when superimposed, using backbone atoms (N, C_{α}, C and 0), on the structure coordinates listed in Table 1 (or Table 2) are considered to be identical with the structure coordinates listed in the Table 1 (or Table 2) when at least about 50% to 100% of the backbone atoms of PYK2 or PYK2 kinase domain are included in the superposition.

V. Uses of the Crystals and Atomic Structure Coordinates

The crystals of the invention, and particularly the atomic structure coordinates obtained therefrom, have a wide variety of uses. For example, the crystals described herein can be used as a starting point in any of the methods of use for kinases known in the art or later developed. Such methods of use include, for example, identifying molecules that bind to the native or mutated catalytic domain of kinases. The crystals and structure coordinates are particularly useful for identifying ligands that modulate kinase activity as an approach towards developing new therapeutic agents. In particular, the crystals and structural information are useful in methods for ligand development utilizing molecular scaffolds.

The structure coordinates described herein can be used as phasing models or homology models for determining the crystal structures of additional kinases, as well as the structures of co-crystals of such kinases with ligands such as inhibitors, agonists, antagonists, and other molecules. The structure coordinates, as well as models of the three-dimensional structures obtained therefrom, can also be used to aid the elucidation of solution-based structures of native or mutated kinases, such as those obtained via NMR.

VI. Electronic Representations of Kinase Structures

Structural information of kinases or portions of kinases (e.g., kinase active sites) can be represented in many different ways. Particularly useful are electronic representations, as such representations allow rapid and convenient data manipulations and structural modifications. Electronic representations can be embedded in many different storage or memory media, frequently computer readable media. Examples include without limitations, computer random access memory (RAM), floppy disk, magnetic hard drive, magnetic tape (analog or digital), compact disk (CD), optical disk, CD-ROM, memory card, digital video disk (DVD), and others. The storage medium can be separate or part of a computer system. Such a computer system may be a dedicated, special purpose, or embedded system, such as a computer system that forms part of an X-ray crystallography system, or may be a general purpose computer (which may have data connection with other equipment such as a sensor device in an X-ray crystallographic system. In many cases, the information provided by such electronic representations can also be represented physically or visually in two or three dimensions, e.g., on paper, as a visual display (e.g., on a computer monitor as a two-dimensional or pseudo-three-dimensional image) or as a three-dimensional physical model. Such physical representations can also be used, alone or in connection with electronic representations. Exemplary useful representations include, but are not limited to, the following:

Atomic Coordinate Representation

One type of representation is a list or table of atomic coordinates representing positions of particular atoms in a molecular structure, portions of a structure, or complex (e.g., a co-crystal). Such a representation may also include additional information, for example, information about occupancy of particular coordinates. One such atomic coordinate representation contains the coordinate information of Table 1 in electronic form.

Energy Surface or Surface of Interaction Representation

Another representation is an energy surface representation, e.g., of an active site or other binding site, representing an energy surface for electronic and steric interactions. Such a representation may also include other features. An example is the inclusion of representation of a particular amino acid residue(s) or group(s) on a particular amino acid residue(s), e.g., a residue or group that can participate in H-bonding or ionic interaction. Such energy surface representations can be readily generated from atomic coordinate representations using any of a variety of available computer programs.

Structural Representation

Still another representation is a structural representation, i.e., a physical representation or an electronic representation of such a physical representation. Such a structural representation includes representations of relative positions of particular features of a molecule or complex, often with linkage between structural features. For example, a structure can be represented in which all atoms are linked; atoms other than hydrogen are linked; backbone atoms, with or without representation of sidechain atoms that could participate in significant electronic interaction, are linked; among others. However, not all features need to be linked. For example, for structural representations of portions of a molecule or complex, structural features significant for that feature may be represented (e.g., atoms of amino acid residues that can have significant binding interation with a ligand at a binding site. Those amino acid residues may not be linked with each other.

A structural representation can also be a schematic representation. For example, a schematic representation can represent secondary and/or tertiary structure in a schematic manner. Within such a schematic representation of a polypeptide, a particular amino acid residue(s) or group(s) on a residue(s) can be included, e.g., conserved residues in a binding site, and/or residue(s) or group(s) that may interact with binding compounds. Electronic structural representations can be generated, for example, from atomic coordinate information using computer programs designed for that function and/or by constructing an electronic representation with manual input based on interpretation of another form of structural information. Physical representations can be created, for example, by printing an image of a computer-generated image, by constructing a 3D model.

VII. Structure Determination for Kinases with Unknown Structure Using Structural Coordinates

Structural coordinates, such as those set forth in Table 1, can be used to determine the three dimensional structures of kinases with unknown structure. The methods described below can apply structural coordinates of a polypeptide with known structure to another data set, such as an amino acid sequence, X-ray crystallographic diffraction data, or nuclear magnetic resonance (NMR) data. Preferred embodiments of the invention relate to determining the three dimensional structures of other serine/threonine kinases, and related polypeptides.

Structures Using Amino Acid Homology

Homology modeling is a method of applying structural coordinates of a polypeptide of known structure to the amino acid sequence of a polypeptide of unknown structure. This method is accomplished using a computer representation of the three dimensional structure of a polypeptide or polypeptide complex, the computer representation of amino acid sequences of the polypeptides with known and unknown structures, and standard computer representations of the structures of amino acids. Homology modeling generally involves (a) aligning the amino acid sequences of the polypeptides with and without known structure; (b) transferring the coordinates of the conserved amino acids in the known structure to the corresponding amino acids of the polypeptide of unknown structure; refining the subsequent three dimensional structure; and (d) constructing structures of the rest of the polypeptide. One skilled in the art recognizes that conserved amino acids between two proteins can be determined from the sequence alignment step in step (a).

The above method is well known to those skilled in the art. (Greer (1985) Science 228:1055; Blundell et al. A(1988) Eur. J. Biochem. 172:513. An exemplary computer program that can be utilized for homology modeling by those skilled in the art is the Homology module in the Insight II modeling package distributed by Accelerys Inc.

Alignment of the amino acid sequence is accomplished by first placing the computer representation of the amino acid sequence of a polypeptide with known structure above the amino acid sequence of the polypeptide of unknown structure. Amino acids in the sequences are then compared and groups of amino acids that are homologous (e.g., amino acid side chains that are similar in chemical nature—aliphatic, aromatic, polar, or charged) are grouped together. This method will detect conserved regions of the polypeptides and account for amino acid insertions or deletions. Such alignment and/or can also be performed fully electronically using sequence alignment and analyses software.

Once the amino acid sequences of the polypeptides with known and unknown structures are aligned, the structures of the conserved amino acids in the computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions are to be assigned manually by either using standard peptide geometries or molecular simulation techniques, such as molecular dynamics. The final step in the process is accomplished by refining the entire structure using molecular dynamics and/or energy minimization. The homology modeling method is well known to those skilled in the art and has been practiced using different protein molecules. For example, the three dimensional structure of the polypeptide corresponding to the catalytic domain of a serine/threonine protein kinase, myosin light chain protein kinase, was homology modeled from the cAMP-dependent protein kinase catalytic subunit. (Knighton et al. (1992) Science 258:130-135.)

Structures Using Molecular Replacement

Molecular replacement is a method of applying the X-ray diffraction data of a polypeptide of known structure to the X-ray diffraction data of a polypeptide of unknown sequence. This method can be utilized to define the phases describing the X-ray diffraction data of a polypeptide of unknown structure when only the amplitudes are known. X-PLOR is a commonly utilized computer software package used for molecular replacement. Brunger (1992) Nature 355:472-475. AMORE is another program used for molecular replacement. Navaza (1994) Acta Crystallogr. A50:157-163. Preferably, the resulting structure does not exhibit a root-mean-square deviation of more than 3 Å.

A goal of molecular replacement is to align the positions of atoms in the unit cell by matching electron diffraction data from two crystals. A program such as X-PLOR can involve four steps. A first step can be to determine the number of molecules in the unit cell and define the angles between them. A second step can involve rotating the diffraction data to define the orientation of the molecules in the unit cell. A third step can be to translate the electron density in three dimensions to correctly position the molecules in the unit cell. Once the amplitudes and phases of the X-ray diffraction data is determined, an R-factor can be calculated by comparing electron diffraction maps calculated experimentally from the reference data set and calculated from the new data set. An R-factor between 30-50% indicates that the orientations of the atoms in the unit cell are reasonably determined by this method. A fourth step in the process can be to decrease the R-factor to roughly 20% by refining the new electron density map using iterative refinement techniques described herein and known to those or ordinary skill in the art.

Structures Using NMR Data

Structural coordinates of a polypeptide or polypeptide complex derived from X-ray crystallographic techniques can be applied towards the elucidation of three dimensional structures of polypeptides from nuclear magnetic resonance (NMR) data. This method is used by those skilled in the art. (Wuthrich, (1986), John Wiley and Sons, New York: 176-199; Pflugrath et al. (1986) J. Mol. Biol. 189:383-386; Kline et al. (1986) J. Mol. Biol. 189:377-382.) While the secondary structure of a polypeptide is often readily determined by utilizing two-dimensional NMR data, the spatial connections between individual pieces of secondary structure are not as readily determinable. The coordinates defining a three-dimensional structure of a polypeptide derived from X-ray crystallographic techniques can guide the NMR spectroscopist to an understanding of these spatial interactions between secondary structural elements in a polypeptide of related structure.

The knowledge of spatial interactions between secondary structural elements can greatly simplify Nuclear Overhauser Effect (NOE) data from two-dimensional NMR experiments. Additionally, applying the crystallographic coordinates after the determination of secondary structure by NMR techniques only simplifies the assignment of NOEs relating to particular amino acids in the polypeptide sequence and does not greatly bias the NMR analysis of polypeptide structure. Conversely, using the crystallographic coordinates to simplify NOE data while determining secondary structure of the polypeptide would bias the NMR analysis of protein structure.

VIII. Structure-Based Design of Modulators of Kinase Function Utilizing Structural Coordinates

Structure-based modulator design and identification methods are powerful techniques that can involve searches of computer databases containing a wide variety of potential modulators and chemical functional groups. The computerized design and identification of modulators is useful as the computer databases contain more compounds than the chemical libraries, often by an order of magnitude. For reviews of structure-based drug design and identification (see Kuntz et al. (1994), Acc. Chem. Res. 27:117; Guida (1994) Current Opinion in Struc. Biol. 4: 777; Colman (1994) Current Opinion in Struc. Biol. 4: 868).

The three dimensional structure of a polypeptide defined by structural coordinates can be utilized by these design methods, for example, the structural coordinates of Table 1. In addition, the three dimensional structures of kinases determined by the homology, molecular replacement, and NMR techniques described herein can also be applied to modulator design and identification methods.

For identifying modulators, structural information for a native kinase, in particular, structural information for the active site of the kinase, can be used. However, it may be advantageous to utilize structural information from one or more co-crystals of the kinase with one or more binding compounds. It can also be advantageous if the binding compound has a structural core in common with test compounds.

Design by Searching Molecular Data Bases

One method of rational design searches for modulators by docking the computer representations of compounds from a database of molecules. Publicly available databases include, for example:
a) ACD from Molecular Designs Limited
b) NCI from National Cancer Institute
c) CCDC from Cambridge Crystallographic Data Center
d) CAST from Chemical Abstract Service
e) Derwent from Derwent Information Limited
f) Maybridge from Maybridge Chemical Company LTD
g) Aldrich from Aldrich Chemical Company
h) Directory of Natural Products from Chapman & Hall

One such data base (ACD distributed by Molecular Designs Limited Information Systems) contains compounds that are synthetically derived or are natural products. Methods available to those skilled in the art can convert a data set represented in two dimensions to one represented in three dimensions. These methods are enabled by such computer programs as CONCORD from Tripos Associates or DE-Converter from Molecular Simulations Limited.

Multiple methods of structure-based modulator design are known to those in the art. (Kuntz et al., (1982), J. Mol. Biol. 162: 269; Kuntz et aZ., (1994), Acc. Chern. Res. 27: 117; Meng et al., (1992), J. Compt. Chem. 13: 505; Bohm, (1994), J. Comp. Aided Molec. Design 8: 623.)

A computer program widely utilized by those skilled in the art of rational modulator design is DOCK from the University of California in San Francisco. The general methods utilized by this computer program and programs like it are described in three applications below. More detailed information regarding some of these techniques can be found in the Accelerys User Guide, 1995. A typical computer program used for this purpose can perform a processes comprising the following steps or functions:
(a) remove the existing compound from the protein;
(b) dock the structure of another compound into the active-site using the computer program (such as DOCK) or by interactively moving the compound into the active-site;
(c) characterize the space between the compound and the active-site atoms;
(d) search libraries for molecular fragments which (i) can fit into the empty space between the compound and the active-site, and (ii) can be linked to the compound; and
(e) link the fragments found above to the compound and evaluate the new modified compound.

Part (c) refers to characterizing the geometry and the complementary interactions formed between the atoms of the active site and the compounds. A favorable geometric fit is attained when a significant surface area is shared between the compound and active-site atoms without forming unfavorable steric interactions. One skilled in the art would note that the method can be performed by skipping parts (d) and (e) and screening a database of many compounds.

Structure-based design and identification of modulators of kinase function can be used in conjunction with assay screening. As large computer databases of compounds (around 10,000 compounds) can be searched in a matter of hours or even less, the computer-based method can narrow the compounds tested as potential modulators of kinase function in biochemical or cellular assays.

The above descriptions of structure-based modulator design are not all encompassing and other methods are reported in the literature and can be used, e.g.:
(1) CAVEAT: Bartlett et al., (1989), in Chemical and Biological Problems in Molecular Recognition, Roberts, S. M.; Ley, S. V.; Campbell, M. M. eds.; Royal Society of Chemistry: Cambridge, pp.182-196.

(2) FLOG: Miller et al., (1994), J. Comp. Aided Molec. Design 8:153.

(3) PRO Modulator: Clark et al., (1995), J. Comp. Aided Molec. Design 9:13.

(4) MCSS: Miranker and Karplus, (1991), Proteins: Structure, Function, and Genetics 11:29.

(5) AUTODOCK: Goodsell and Olson, (1990), Proteins: Structure, Function, and Genetics 8:195.

(6) GRID: Goodford, (1985), J. Med. Chem. 28:849.

Design by Modifying Compounds in Complex with PYK2 Kinase

Another way of identifying compounds as potential modulators is to modify an existing modulator in the polypeptide active site. For example, the computer representation of modulators can be modified within the computer representation of a PYK2 active site. Detailed instructions for this technique can be found, for example, in the Accelerys User Manual, 1995 in LUDI. The computer representation of the modulator is typically modified by the deletion of a chemical group or groups or by the addition of a chemical group or groups.

Upon each modification to the compound, the atoms of the modified compound and active site can be shifted in conformation and the distance between the modulator and the active-site atoms may be scored along with any complementary interactions formed between the two molecules. Scoring can be complete when a favorable geometric fit and favorable complementary interactions are attained. Compounds that have favorable scores are potential modulators.

Design by Modifying the Structure of Compounds that Bind PYK2 Kinase

A third method of structure-based modulator design is to screen compounds designed by a modulator building or modulator searching computer program. Examples of these types of programs can be found in the Molecular Simulations Package, Catalyst. Descriptions for using this program are documented in the Molecular Simulations User Guide (1995). Other computer programs used in this application are ISIS/HOST, ISIS/BASE, ISIS/DRAW) from Molecular Designs Limited and UNITY from Tripos Associates.

These programs can be operated on the structure of a compound that has been removed from the active site of the three dimensional structure of a compound-kinase complex. Operating the program on such a compound is preferable since it is in a biologically active conformation.

A modulator construction computer program is a computer program that may be used to replace computer representations of chemical groups in a compound complexed with a kinase or other biomolecule with groups from a computer database. A modulator searching computer program is a computer program that may be used to search computer representations of compounds from a computer data base that have similar three dimensional structures and similar chemical groups as compound bound to a particular biomolecule.

A typical program can operate by using the following general steps:
(a) map the compounds by chemical features such as by hydrogen bond donors or acceptors, hydrophobic/lipophilic sites, positively ionizable sites, or negatively ionizable sites;
(b) add geometric constraints to the mapped features; and
(c) search databases with the model generated in (b).

Those skilled in the art also recognize that not all of the possible chemical features of the compound need be present in the model of (b). One can use any subset of the model to generate different models for data base searches.

Modulator Design Using Molecular Scaffolds

The present invention can also advantageously utilize methods for designing compounds, designated as molecular scaffolds, that can act broadly across families of molecules and/or for using a molecular scaffold to design ligands that target individual or multiple members of those families. In preferred embodiments, the molecules can be proteins and a set of chemical compounds can be assembled that have properties such that they are 1) chemically designed to act on certain protein families and/or 2) behave more like molecular scaffolds, meaning that they have chemical substructures that make them specific for binding to one or more proteins in a family of interest. Alternatively, molecular scaffolds can be designed that are preferentially active on an individual target molecule.

Useful chemical properties of molecular scaffolds can include one or more of the following characteristics, but are not limited thereto: an average molecular weight below about 350 daltons, or between from about 150 to about 350 daltons, or from about 150 to about 300 daltons; having a clogP below 3; a number of rotatable bonds of less than 4; a number of hydrogen bond donors and acceptors below 5 or below 4; a polar surface area of less than 50 Å²; binding at protein binding sites in an orientation so that chemical substituents from a combinatorial library that are attached to the scaffold can be projected into pockets in the protein binding site; and possessing chemically tractable structures at its substituent attachment points that can be modified, thereby enabling rapid library construction.

By “clog P” is meant the calculated log P of a compound, “P” referring to the partition coefficient between octanol and water.

The term “Molecular Polar Surface Area (PSA)” refers to the sum of surface contributions of polar atoms (usually oxygens, nitrogens and attached hydrogens) in a molecule. The polar surface area has been shown to correlate well with drug transport properties, such as intestinal absorption, or blood-brain barrier penetration.

Additional useful chemical properties of distinct compounds for inclusion in a combinatorial library include the ability to attach chemical moieties to the compound that will not interfere with binding of the compound to at least one protein of interest, and that will impart desirable properties to the library members, for example, causing the library members to be actively transported to cells and/or organs of interest, or the ability to attach to a device such as a chromatography column (e.g., a streptavidin column through a molecule such as biotin) for uses such as tissue and proteomics profiling purposes.

A person of ordinary skill in the art will realize other properties that can be desirable for the scaffold or library members to have depending on the particular requirements of the use, and that compounds with these properties can also be sought and identified in like manner. Methods of selecting compounds for assay are known to those of ordinary skill in the art, for example, methods and compounds described in U.S. Pat. Nos. 6,288,234, 6,090,912, 5,840,485, each of which is hereby incorporated by reference in its entirety, including all charts and drawings.

In various embodiments, the present invention provides methods of designing ligands that bind to a plurality of members of a molecular family, where the ligands contain a common molecular scaffold. Thus, a compound set can be assayed for binding to a plurality of members of a molecular family, e.g., a protein family. One or more compounds that bind to a plurality of family members can be identified as molecular scaffolds. When the orientation of the scaffold at the binding site of the target molecules has been determined and chemically tractable structures have been identified, a set of ligands can be synthesized starting with one or a few molecular scaffolds to arrive at a plurality of ligands, wherein each ligand binds to a separate target molecule of the molecular family with altered or changed binding affinity or binding specificity relative to the scaffold. Thus, a plurality of drug lead molecules can be designed to preferentially target individual members of a molecular family based on the same molecular scaffold, and act on them in a specific manner.

IX. Binding Assays

The methods of the present invention can involve assays that are able to detect the binding of compounds to a target molecule. Such binding is at a statistically significant level, preferably with a confidence level of at least 90%, more preferably at least 95, 97, 98, 99% or greater confidence level that the assay signal represents binding to the target molecule, i.e., is distinguished from background. Preferably controls are used to distinguish target binding from non-specific binding. The assays of the present invention can also include assaying compounds for low affinity binding to the target molecule. A large variety of assays indicative of binding are known for different target types and can be used for this invention. Compounds that act broadly across protein families are not likely to have a high affinity against individual targets, due to the broad nature of their binding. Thus, assays described herein allow for the identification of compounds that bind with low affinity, very low affinity, and extremely low affinity. Therefore, potency (or binding affinity) is not the primary, nor even the most important, indicia of identification of a potentially useful binding compound. Rather, even those compounds that bind with low affinity, very low affinity, or extremely low affinity can be considered as molecular scaffolds that can continue to the next phase of the ligand design process.

By binding with “low affinity” is meant binding to the target molecule with a dissociation constant (k_{d}) of greater than 1 μM under standard conditions. By binding with “very low affinity” is meant binding with a kd of above about 100 μM under standard conditions. By binding with “extremely low affinity” is meant binding at a k_{d} of above about 1 mM under standard conditions. By “moderate affinity” is meant binding with a k_{d} of from about 200 nM to about 1 μM under standard conditions. By “moderately high affinity” is meant binding at a k_{d} of from about 1 nM to about 200 nM. By binding at “high affinity” is meant binding at a k_{d} of below about 1 nM under standard conditions. For example, low affinity binding can occur because of a poorer fit into the binding site of the target molecule or because of a smaller number of non-covalent bonds, or weaker covalent bonds present to cause binding of the scaffold or ligand to the binding site of the target molecule relative to instances where higher affinity binding occurs. The standard conditions for binding are at pH 7.2 at 37° C. for one hour. For example, 100 μl/well can be used in HEPES 50 mM buffer at pH 7.2, NaCl 15 mM, ATP 2 μM, and bovine serum albumin 1 ug/well, 37° C. for one hour.

Binding compounds can also be characterized by their effect on the activity of the target molecule. Thus, a “low activity” compound has an inhibitory concentration (IC₅₀) or excitation concentration (EC₅₀) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667$$

To design or discover scaffolds that act broadly across protein families, proteins of interest can be assayed against a compound collection or set. The assays can preferably be enzymatic or binding assays. In some embodiments it may be desirable to enhance the solubility of the compounds being screened and then analyze all compounds that show activity in the assay, including those that bind with low affinity or produce a signal with greater than about three times the standard deviation of the background signal. The assays can be any suitable assay such as, for example, binding assays that measure the binding affinity between two binding partners. Various types of screening assays that can be useful in the practice of the present invention are known in the art, such as those described in U.S. Pat. Nos. 5,763,198, 5,747,276, 5,877,007, 6,243,980, 6,294,330, and 6,294,330, each of which is hereby incorporated by reference in its entirety, including all charts and drawings.

In various embodiments of the assays at least one compound, at least about 5%, at least about 10%, at least about 15%, at least about 20%, or at least about 25% of the compounds can bind with low affinity. In general, up to about 20% of the compounds can show activity in the screening assay and these compounds can then be analyzed directly with high-throughput co-crystallography, computational analysis to group the compounds into classes with common structural properties (e.g., structural core and/or shape and polarity characteristics), and the identification of common chemical structures between compounds that show activity.

The person of ordinary skill in the art will realize that decisions can be based on criteria that are appropriate for the needs of the particular situation, and that the decisions can be made by computer software programs. Classes can be created containing almost any number of scaffolds, and the criteria selected can be based on increasingly exacting criteria until an arbitrary number of scaffolds is arrived at for each class that is deemed to be advantageous.

Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference. Vely F. et al., (2000) BIAcore® analysis to test phosphopeptide-SH2 domain interactions, Methods in Molecular Biology. 121:313-21; Liparoto et al., (1999) Biosensor analysis of the interleukin-2 receptor complex, Journal of Molecular Recognition. 12:316-21; Lipschultz et al., (2000) Experimental design for analysis of complex kinetics using surface plasmon resonance, Methods. 20(3):310-8; Malmqvist., (1999) BIACORE: an affinity biosensor system for characterization of biomolecular interactions, Biochemical Society Transactions 27:335-40; Alfthan, (1998) Surface plasmon resonance biosensors as a tool in antibody engineering, Biosensors & Bioelectronics. 13:653-63; Fivash et al., (1998) BIAcore for macromolecular interaction, Current Opinion in Biotechnology. 9:97-101; Price et al.; (1998) Summary report on the ISOBM TD-4 Workshop: analysis of 56 monoclonal antibodies against the MUC 1 mucin. Tumour Biology 19 Suppl 1:1-20; Malmqvist et al, (1997) Biomolecular interaction analysis: affinity biosensor technologies for functional analysis of proteins, Current Opinion in Chemical Biology. 1:378-83; O'Shannessy et al., (1996) Interpretation of deviations from pseudo-first-order kinetic behavior in the characterization of ligand binding by biosensor technology, Analytical Biochemistry. 236:275-83; Malmborg et al., (1995) BIAcore as a tool in antibody engineering, Journal of Immunological Methods. 183:7-13; Van Regenmortel, (1994) Use of biosensors to characterize recombinant proteins, Developments in Biological Standardization. 83:143-51; and O'Shannessy, (1994) Determination of kinetic rate and equilibrium binding constants for macromolecular interactions: a critique of the surface plasmon resonance literature, Current Opinions in Biotechnology. 5:65-71.

BIAcore® uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator. Similarly, when ligands to a sphingolipid target are sought, known ligands of the target can be present in control/calibration assay wells.

Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of colorimetric assays for the detection of peroxides, as disclosed in Example 1(b) and Gordon, A. J. and Ford, R. A., (1972) The Chemist's Companion: A Handbook Of Practical Data, Techniques, And References, John Wiley and Sons, N.Y., Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., (1987) Spectrophotometry and Spectrofluorometry: A Practical Approach, pp. 91-114, IRL Press Ltd.; and Bell, (1981) Spectroscopy In Biochemistry, Vol. I, pp. 155-194, CRC Press.

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, Oreg.). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex® Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., (1997), Application of Fluorescence Polarization Assays in High-Throughput Screening, Genetic Engineering News, 17:27.

FP is particularly desirable since its readout is independent of the emission intensity (Checovich, W. J., et al., (1995) Nature 375:254-256; Dandliker, W. B., et al., (1981) Methods in Enzymology 74:3-28) and is thus insensitive to the presence of colored compounds that quench fluorescence emission. FP and FRET (see below) are well-suited for identifying compounds that block interactions between sphingolipid receptors and their ligands. See, for example, Parker et al., (2000) Development of high throughput screening assays using fluorescence polarization: nuclear receptor-ligand-binding and kinase/phosphatase assays, J Biomol Screen 5:77-88.

Fluorophores derived from sphingolipids that may be used in FP assays are commercially available. For example, Molecular Probes (Eugene, Oreg.) currently sells sphingomyelin and one ceramide flurophores. These are, respectively, N-(4,4-difluoro-5,7-dimethyl-4-bora-3a,4a-diaza-s-indacene-3-pentanoyl)sphingosyl phosphocholine (BODIPY® FL C5-sphingomyelin); N-(4,4-difluoro-5,7-dimethyl-4-bora-3a,4a-diaza-s-indacene-3-dodecanoyl)sphingosyl phosphocholine (BODIPY® FL C12-sphingomyelin); and N-(4,4-difluoro-5,7-dimethyl-4-bora-3a,4a-diaza-s-indacene-3-pentanoyl)sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION® fluorescence polarization system (Tecan AG, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., (1996) Curr. Biol. 6:178-182; Mitra et al., (1996) Gene 173:13-17; and Selvin et al., (1995) Meth. Enzymol. 246:300-345. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., (1997) J. Lipid Res. 38:2365-2373; Kahl et al., (1996) Anal. Biochem. 243:282-283; Undenfriend et al., (1987) Anal. Biochem. 161:494-500). See also U.S. Pat. Nos. 4,626,513 and 4,568,649, and European Patent No. 0,154,734. One commercially available system uses FLASHPLATE® scintillant-coated plates (NEN Life Science Products, Boston, Mass.).

The target molecule can be bound to the scintillator plates by a variety of well known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT® microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., (1998) Anal. Biochem. 257:112-119).

Assay Compounds and Molecular Scaffolds

Preferred characteristics of a scaffold include being of low molecular weight (e.g., less than 350 Da, or from about 100 to about 350 daltons, or from about 150 to about 300 daltons). Preferably clog P of a scaffold is from −1 to 8, more preferably less than 6, 5, or 4, most preferably less than 3. In particular embodiments the clogP is in a range −1 to an upper limit of 2, 3, 4, 5, 6, or 8; or is in a range of 0 to an upper limit of 2, 3, 4, 5, 6, or 8. Preferably the number of rotatable bonds is less than 5, more preferably less than 4. Preferably the number of hydrogen bond donors and acceptors is below 6, more preferably below 5. An additional criterion that can be useful is a polar surface area of less than 5. Guidance that can be useful in identifying criteria for a particular application can be found in Lipinski et al., (1997) Advanced Drug Delivery Reviews 23 3-25, which is hereby incorporated by reference in its entirety.

A scaffold may preferably bind to a given protein binding site in a configuration that causes substituent moieties of the scaffold to be situated in pockets of the protein binding site. Also, possessing chemically tractable groups that can be chemically modified, particularly through synthetic reactions, to easily create a combinatorial library can be a preferred characteristic of the scaffold. Also preferred can be having positions on the scaffold to which other moieties can be attached, which do not interfere with binding of the scaffold to the protein(s) of interest but do cause the scaffold to achieve a desirable property, for example, active transport of the scaffold to cells and/or organs, enabling the scaffold to be attached to a chromatographic column to facilitate analysis, or another desirable property. A molecular scaffold can bind to a target molecule with any affinity, such as binding at high affinity, moderate affinity, low affinity, very low affinity, or extremely low affinity.

Thus, the above criteria can be utilized to select many compounds for testing that have the desired attributes. Many compounds having the criteria described are available in the commercial market, and may be selected for assaying depending on the specific needs to which the methods are to be applied.

A “compound library” or “library” is a collection of different compounds having different chemical structures. A compound library is screenable, that is, the compound library members therein may be subject to screening assays. In preferred embodiments, the library members can have a molecular weight of from about 100 to about 350 daltons, or from about 150 to about 350 daltons. Examples of libraries are provided above.

Libraries of the present invention can contain at least one compound than binds to the target molecule at low affinity. Libraries of candidate compounds can be assayed by many different assays, such as those described above, e.g., a fluorescence polarization assay. Libraries may consist of chemically synthesized peptides, peptidomimetics, or arrays of combinatorial chemicals that are large or small, focused or nonfocused. By “focused” it is meant that the collection of compounds is prepared using the structure of previously characterized compounds and/or pharmacophores.

Compound libraries may contain molecules isolated from natural sources, artificially synthesized molecules, or molecules synthesized, isolated, or otherwise prepared in such a manner so as to have one or more moieties variable, e.g., moieties that are independently isolated or randomly synthesized. Types of molecules in compound libraries include but are not limited to organic compounds, polypeptides and nucleic acids as those terms are used herein, and derivatives, conjugates and mixtures thereof.

Compound libraries of the invention may be purchased on the commercial market or prepared or obtained by any means including, but not limited to, combinatorial chemistry techniques, fermentation methods, plant and cellular extraction procedures and the like (see, e.g., Cwirla et al., (1990) Biochemistry, 87, 6378-6382; Houghten et al., (1991) Nature, 354, 84-86; Lam et al., (1991) Nature, 354, 82-84; Brenner et al., (1992) Proc. Natl. Acad. Sci. USA, 89, 5381-5383; R. A. Houghten, (1993) Trends Genet., 9, 235-239; E. R. Felder, (1994) Chimia, 48, 512-541; Gallop et al., (1994) J. Med. Chem., 37, 1233-1251; Gordon et al., (1994) J. Med. Chem., 37, 1385-1401; Carell et al., (1995) Chem. Biol., 3, 171-183; Madden et al., Perspectives in Drug Discovery and Design 2, 269-282; Lebl et al., (1995) Biopolymers, 37 177-198); small molecules assembled around a shared molecular structure; collections of chemicals that have been assembled by various commercial and noncommercial groups, natural products; extracts of marine organisms, fungi, bacteria, and plants.

Preferred libraries can be prepared in a homogenous reaction mixture, and separation of unreacted reagents from members of the library is not required prior to screening. Although many combinatorial chemistry approaches are based on solid state chemistry, liquid phase combinatorial chemistry is capable of generating libraries (Sun C M., (1999) Recent advances in liquid-phase combinatorial chemistry, Combinatorial Chemistry & High Throughput Screening. 2:299-318).

Libraries of a variety of types of molecules are prepared in order to obtain members therefrom having one or more preselected attributes that can be prepared by a variety of techniques, including but not limited to parallel array synthesis (Houghton, (2000) Annu Rev Pharmacol Toxicol 40:273-82, Parallel array and mixture-based synthetic combinatorial chemistry; solution-phase combinatorial chemistry (Merritt, (1998) Comb Chem High Throughput Screen 1(2):57-72, Solution phase combinatorial chemistry, Coe et al., (1998-99) Mol Divers;4(1):31-8, Solution-phase combinatorial chemistry, Sun, (1999) Comb Chem High Throughput Screen 2(6):299-318, Recent advances in liquid-phase combinatorial chemistry); synthesis on soluble polymer (Gravert et al., (1997) Curr Opin Chem Biol 1(1):107-13, Synthesis on soluble polymers: new reactions and the construction of small molecules); and the like. See, e.g., Dolle et al., (1999) J Comb Chem 1(4):235-82, Comprehensive survey of cominatorial library synthesis: 1998. Freidinger R M., (1999) Nonpeptidic ligands for peptide and protein receptors, Current Opinion in Chemical Biology; and Kundu et al., Prog Drug Res; 53:89-156, Combinatorial chemistry: polymer supported synthesis of peptide and non-peptide libraries). Compounds may be clinically tagged for ease of identification (Chabala, (1995) Curr Opin Biotechnol 6(6):633-9, Solid-phase combinatorial chemistry and novel tagging methods for identifying leads).

The combinatorial synthesis of carbohydrates and libraries containing oligosaccharides have been described (Schweizer et al., (1999) Curr Opin Chem Biol 3(3):291-8, Combinatorial synthesis of carbohydrates). The synthesis of natural-product based compound libraries has been described (Wessjohann, (2000) Curr Opin Chem Biol 4(3):303-9, Synthesis of natural-product based compound libraries).

Libraries of nucleic acids are prepared by various techniques, including by way of non-limiting example the ones described herein, for the isolation of aptamers. Libraries that include oligonucleotides and polyaminooligonucleotides (Markiewicz et al., (2000) Synthetic oligonucleotide combinatorial libraries and their applications, Farmaco. 55:174-7) displayed on streptavidin magnetic beads are known. Nucleic acid libraries are known that can be coupled to parallel sampling and be deconvoluted without complex procedures such as automated mass spectrometry (Enjalbal C. Martinez J. Aubagnac J L, (2000) Mass spectrometry in combinatorial chemistry, Mass Spectrometry Reviews. 19:139-61) and parallel tagging. (Perrin D M., Nucleic acids for recognition and catalysis: landmarks, limitations, and looking to the future, Combinatorial Chemistry & High Throughput Screening 3:243-69).

Peptidomimetics are identified using combinatorial chemistry and solid phase synthesis (Kim H O. Kahn M., (2000) A merger of rational drug design and combinatorial chemistry: development and application of peptide secondary structure mimetics, Combinatorial Chemistry & High Throughput Screening 3:167-83; al-Obeidi, (1998) Mol Biotechnol 9(3):205-23, Peptide and peptidomimetric libraries. Molecular diversity and drug design). The synthesis may be entirely random or based in part on a known polypeptide.

Polypeptide libraries can be prepared according to various techniques. In brief, phage display techniques can be used to produce polypeptide ligands (Gram H., (1999) Phage display in proteolysis and signal transduction, Combinatorial Chemistry & High Throughput Screening. 2:19-28) that may be used as the basis for synthesis of peptidomimetics. Polypeptides, constrained peptides, proteins, protein domains, antibodies, single chain antibody fragments, antibody fragments, and antibody combining regions are displayed on filamentous phage for selection.

Large libraries of individual variants of human single chain Fv antibodies have been produced. See, e.g., Siegel R W. Allen B. Pavlik P. Marks J D. Bradbury A., (2000) Mass spectral analysis of a protein complex using single-chain antibodies selected on a peptide target: applications to functional genomics, Journal of Molecular Biology 302:285-93; Poul M A. Becerril B. Nielsen U B. Morisson P. Marks J D., (2000) Selection of tumor-specific internalizing human antibodies from phage libraries. Source Journal of Molecular Biology. 301:1149-61; Amersdorfer P. Marks J D., (2001) Phage libraries for generation of anti-botulinum scFv antibodies, Methods in Molecular Biology. 145:219-40; Hughes-Jones N C. Bye J M. Gorick B D. Marks J D. Ouwehand W H., (1999) Synthesis of Rh Fv phage-antibodies using VH and VL germline genes, British Journal of Haematology. 105:811-6; McCall A M. Amoroso A R. Sautes C. Marks J D. Weiner L M., (1998) Characterization of anti-mouse Fc gamma RII single-chain Fv fragments derived from human phage display libraries, Immunotechnology. 4:71-87; Sheets M D. Amersdorfer P. Finnern R. Sargent P. Lindquist E. Schier R. Hemingsen G. Wong C. Gerhart J C. Marks J D. Lindquist E., (1998) Efficient construction of a large nonimmune phage antibody library: the production of high-affinity human single-chain antibodies to protein antigens (published erratum appears in Proc Natl Acad Sci USA 1999 96:795), Proc Natl Acad Sci USA 95:6157-62).

Focused or smart chemical and pharmacophore libraries can be designed with the help of sophisticated strategies involving computational chemistry (e.g., Kundu B. Khare S K. Rastogi S K., (1999) Combinatorial chemistry: polymer supported synthesis of peptide and non-peptide libraries, Progress in Drug Research 53:89-156) and the use of structure-based ligands using database searching and docking, de novo drug design and estimation of ligand binding affinities (Joseph-McCarthy D., (1999) Computational approaches to structure-based ligand design, Pharmacology & Therapeutics 84:179-91; Kirkpatrick D L. Watson S. Ulhaq S., (1999) Structure-based drug design: combinatorial chemistry and molecular modeling, Combinatorial Chemistry & High Throughput Screening. 2:211-21; Eliseev A V. Lehn J M., (1999) Dynamic combinatorial chemistry: evolutionary formation and screening of molecular libraries, Current Topics in Microbiology & Immunology 243:159-72; Bolger et al., (1991) Methods Enz. 203:21-45; Martin, (1991) Methods Enz. 203:587-613; Neidle et al., (1991) Methods Enz. 203:433-458; U.S. Pat. No. 6,178,384).

X. Crystallography

After binding compounds have been determined, the orientation of compound bound to target is determined. Preferably this determination involves crystallography on co-crystals of molecular scaffold compounds with target. Most protein crystallographic platforms can preferably be designed to analyze up to about 500 co-complexes of compounds, ligands, or molecular scaffolds bound to protein targets due to the physical parameters of the instruments and convenience of operation. If the number of scaffolds that have binding activity exceeds a number convenient for the application of crystallography methods, the scaffolds can be placed into groups based on having at least one common chemical structure or other desirable characteristics, and representative compounds can be selected from one or more of the classes. Classes can be made with increasingly exacting criteria until a desired number of classes (e.g., 500) is obtained. The classes can be based on chemical structure similarities between molecular scaffolds in the class, e.g., all possess a pyrrole ring, benzene ring, or other chemical feature. Likewise, classes can be based on shape characteristics, e.g., space-filling characteristics.

The co-crystallography analysis can be performed by co-complexing each scaffold with its target at concentrations of the scaffold that showed activity in the screening assay. This co-complexing can be accomplished with the use of low percentage organic solvents with the target molecule and then concentrating the target with each of the scaffolds. In preferred embodiments these solvents are less than 5% organic solvent such as dimethyl sulfoxide (DMSO), ethanol, methanol, or ethylene glycol in water or another aqueous solvent. Each scaffold complexed to the target molecule can then be screened with a suitable number of crystallization screening conditions at both 4 and 20 degrees. In preferred embodiments, about 96 crystallization screening conditions can be performed in order to obtain sufficient information about the co-complexation and crystallization conditions, and the orientation of the scaffold at the binding site of the target molecule. Crystal structures can then be analyzed to determine how the bound scaffold is oriented physically within the binding site or within one or more binding pockets of the molecular family member.

It is desirable to determine the atomic coordinates of the compounds bound to the target proteins in order to determine which is a most suitable scaffold for the protein family. X-ray crystallographic analysis is therefore most preferable for determining the atomic coordinates. Those compounds selected can be further tested with the application of medicinal chemistry. Compounds can be selected for medicinal chemistry testing based on their binding position in the target molecule. For example, when the compound binds at a binding site, the compound's binding position in the binding site of the target molecule can be considered with respect to the chemistry that can be performed on chemically tractable structures or sub-structures of the compound, and how such modifications on the compound might interact with structures or sub-structures on the binding site of the target. Thus, one can explore the binding site of the target and the chemistry of the scaffold in order to make decisions on how to modify the scaffold to arrive at a ligand with higher potency and/or selectivity. This process allows for more direct design of ligands, by utilizing structural and chemical information obtained directly from the co-complex, thereby enabling one to more efficiently and quickly design lead compounds that are likely to lead to beneficial drug products. In various embodiments it may be desirable to perform co-crystallography on all scaffolds that bind, or only those that bind with a particular affinity, for example, only those that bind with high affinity, moderate affinity, low affinity, very low affinity, or extremely low affinity. It may also be advantageous to perform co-crystallography on a selection of scaffolds that bind with any combination of affinities.

Standard X-ray protein diffraction studies such as by using a Rigaku RU-200® (Rigaku, Tokyo, Japan) with an X-ray imaging plate detector or a synchrotron beam-line can be performed on co-crystals and the diffraction data measured on a standard X-ray detector, such as a CCD detector or an X-ray imaging plate detector.

Performing X-ray crystallography on about 200 co-crystals should generally lead to about 50 co-crystals structures, which should provide about 10 scaffolds for validation in chemistry, which should finally result in about 5 selective leads for target molecules.

Virtual Assays

Commercially available software that generates three-dimensional graphical representations of the complexed target and compound from a set of coordinates provided can be used to illustrate and study how a compound is oriented when bound to a target. (e.g., QUANTA®, Accelerys, San Diego, Calif.). Thus, the existence of binding pockets at the binding site of the targets can be particularly useful in the present invention. These binding pockets are revealed by the crystallographic structure determination and show the precise chemical interactions involved in binding the compound to the binding site of the target. The person of ordinary skill will realize that the illustrations can also be used to decide where chemical groups might be added, substituted, modified, or deleted from the scaffold to enhance binding or another desirable effect, by considering where unoccupied space is located in the complex and which chemical substructures might have suitable size and/or charge characteristics to fill it. The person of ordinary skill will also realize that regions within the binding site can be flexible and its properties can change as a result of scaffold binding, and that chemical groups can be specifically targeted to those regions to achieve a desired effect. Specific locations on the molecular scaffold can be considered with reference to where a suitable chemical substructure can be attached and in which conformation, and which site has the most advantageous chemistry available.

An understanding of the forces that bind the compounds to the target proteins reveals which compounds can most advantageously be used as scaffolds, and which properties can most effectively be manipulated in the design of ligands. The person of ordinary skill will realize that steric, ionic, hydrogen bond, and other forces can be considered for their contribution to the maintenance or enhancement of the target-compound complex. Additional data can be obtained with automated computational methods, such as docking and/or Free Energy Perturbations (FEP), to account for other energetic effects such as desolvation penalties. The compounds selected can be used to generate information about the chemical interactions with the target or for elucidating chemical modifications that can enhance selectivity of binding of the compound.

Computer models, such as homology models (i.e., based on a known, experimentally derived structure) can be constructed using data from the co-crystal structures. When the target molecule is a protein or enzyme, preferred co-crystal structures for making homology models contain high sequence identity in the binding site of the protein sequence being modeled, and the proteins will preferentially also be within the same class and/or fold family. Knowledge of conserved residues in active sites of a protein class can be used to select homology models that accurately represent the binding site. Homology models can also be used to map structural information from a surrogate protein where an apo or co-crystal structure exists to the target protein.

Virtual screening methods, such as docking, can also be used to predict the binding configuration and affinity of scaffolds, compounds, and/or combinatorial library members to homology models. Using this data, and carrying out “virtual experiments” using computer software can save substantial resources and allow the person of ordinary skill to make decisions about which compounds can be suitable scaffolds or ligands, without having to actually synthesize the ligand and perform co-crystallization. Decisions thus can be made about which compounds merit actual synthesis and co-crystallization. An understanding of such chemical interactions aids in the discovery and design of drugs that interact more advantageously with target proteins and/or are more selective for one protein family member over others. Thus, applying these principles, compounds with superior properties can be discovered.

Additives that promote co-crystallization can of course be included in the target molecule formulation in order to enhance the formation of co-crystals. In the case of proteins or enzymes, the scaffold to be tested can be added to the protein formulation, which is preferably present at a concentration of approximately 1 mg/ml. The formulation can also contain between 0%-10% (v/v) organic solvent, e.g. DMSO, methanol, ethanol, propane diol, or 1,3 dimethyl propane diol (MPD) or some combination of those organic solvents. Compounds are preferably solubilized in the organic solvent at a concentration of about 10 mM and added to the protein sample at a concentration of about 100 mM. The protein-compound complex is then concentrated to a final concentration of protein of from about 5 to about 20 mg/ml. The complexation and concentration steps can conveniently be performed using a 96-well formatted concentration apparatus (e.g., Amicon Inc., Piscataway, N.J.). Buffers and other reagents present in the formulation being crystallized can contain other components that promote crystallization or are compatible with crystallization conditions, such as DTT, propane diol, glycerol.

The crystallization experiment can be set-up by placing small aliquots of the concentrated protein-compound complex (1 μl) in a 96 well format and sampling under 96 crystallization conditions. (Other screening formats can also be used, e.g., plates with greater than 96 wells.) Crystals can typically be obtained using standard crystallization protocols that can involve the 96 well crystallization plate being placed at different temperatures. Co-crystallization varying factors other than temperature can also be considered for each protein-compound complex if desirable. For example, atmospheric pressure, the presence or absence of light or oxygen, a change in gravity, and many other variables can all be tested. The person of ordinary skill in the art will realize other variables that can advantageously be varied and considered.

Ligand Design and Preparation

The design and preparation of ligands can be performed with or without structural and/or co-crystallization data by considering the chemical structures in common between the active scaffolds of a set. In this process structure-activity hypotheses can be formed and those chemical structures found to be present in a substantial number of the scaffolds, including those that bind with low affinity, can be presumed to have some effect on the binding of the scaffold. This binding can be presumed to induce a desired biochemical effect when it occurs in a biological system (e.g., a treated mammal). New or modified scaffolds or combinatorial libraries derived from scaffolds can be tested to disprove the maximum number of binding and/or structure-activity hypotheses. The remaining hypotheses can then be used to design ligands that achieve a desired binding and biochemical effect.

But in many cases it will be preferred to have co-crystallography data for consideration of how to modify the scaffold to achieve the desired binding effect (e.g., binding at higher affinity or with higher selectivity). Using the case of proteins and enzymes, co-crystallography data shows the binding pocket of the protein with the molecular scaffold bound to the binding site, and it will be apparent that a modification can be made to a chemically tractable group on the scaffold. For example, a small volume of space at a protein binding site or pocket might be filled by modifying the scaffold to include a small chemical group that fills the volume. Filling the void volume can be expected to result in a greater binding affinity, or the loss of undesirable binding to another member of the protein family. Similarly, the co-crystallography data may show that deletion of a chemical group on the scaffold may decrease a hindrance to binding and result in greater binding affinity or specificity.

It can be desirable to take advantage of the presence of a charged chemical group located at the binding site or pocket of the protein. For example, a positively charged group can be complemented with a negatively charged group introduced on the molecular scaffold. This can be expected to increase binding affinity or binding specificity, thereby resulting in a more desirable ligand. In many cases, regions of protein binding sites or pockets are known to vary from one family member to another based on the amino acid differences in those regions. Chemical additions in such regions can result in the creation or elimination of certain interactions (e.g., hydrophobic, electrostatic, or entropic) that allow a compound to be more specific for one protein target over another or to bind with greater affinity, thereby enabling one to synthesize a compound with greater selectivity or affinity for a particular family member. Additionally, certain regions can contain amino acids that are known to be more flexible than others. This often occurs in amino acids contained in loops connecting elements of the secondary structure of the protein, such as alpha helices or beta strands. Additions of chemical moieties can also be directed to these flexible regions in order to increase the likelihood of a specific interaction occurring between the protein target of interest and the compound. Virtual screening methods can also be conducted in silico to assess the effect of chemical additions, subtractions, modifications, and/or substitutions on compounds with respect to members of a protein family or class.

The addition, subtraction, or modification of a chemical structure or sub-structure to a scaffold can be performed with any suitable chemical moiety. For example the following moieties, which are provided by way of example and are not intended to be limiting, can be utilized: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (e.g., forming a ketone or N-oxide) or a sulphur atom (e.g., forming a thiol, thione, di-alkylsulfoxide or sulfone) are all examples of moieties that can be utilized.

Additional examples of structures or sub-structures that may be utilized are an aryl optionally substituted with one, two, or three substituents independently selected from the group consisting of alkyl, alkoxy, halogen, trihalomethyl, carboxylate, carboxamide, nitro, and ester moieties; an amine of formula -NX₂X₃, where X₂ and X₃ are independently selected from the group consisting of hydrogen, saturated or unsaturated alkyl, and homocyclic or heterocyclic ring moieties; halogen or trihalomethyl; a ketone of formula —COX4, where X₄ is selected from the group consisting of alkyl and homocyclic or heterocyclic ring moieties; a carboxylic acid of formula —(X₅)ₙCOOH or ester of formula (X₆)ₙCOOX₇, where X₅, X₆, and X₇ and are independently selected from the group consisting of alkyl and homocyclic or heterocyclic ring moieties and where n is 0 or 1; an alcohol of formula (X₈)ₙOH or an alkoxy moiety of formula —(X₈)ₙOX₉, where X₈ and X₉ are independently selected from the group consisting of saturated or unsaturated alkyl and homocyclic or heterocyclic ring moieties, wherein said ring is optionally substituted with one or more substituents independently selected from the group consisting of alkyl, alkoxy, halogen, trihalomethyl, carboxylate, nitro, and ester and where n is 0 or 1; an amide of formula NHCOX₁₀, where X₁₀ is selected from the group consisting of alkyl, hydroxyl, and homocyclic or heterocyclic ring moieties, wherein said ring is optionally substituted with one or more substituents independently selected from the group consisting of alkyl, alkoxy, halogen, trihalomethyl, carboxylate, nitro, and ester; SO₂, NX₁₁X₁₂, where X₁₁ and X₁₂ are selected from the group consisting of hydrogen, alkyl, and homocyclic or heterocyclic ring moieties; a homocyclic or heterocyclic ring moiety optionally substituted with one, two, or three substituents independently selected from the group consisting of alkyl, alkoxy, halogen, trihalomethyl, carboxylate, carboxamide, nitro, and ester moieties; an aldehyde of formula —CHO; a sulfone of formula —SO₂X₁₃, where X₁₃ is selected from the group consisting of saturated or unsaturated alkyl and homocyclic or heterocyclic ring moieties; and a nitro of formula —NO₂.

Identification of Attachment Sites on Molecular Scaffolds and Ligands

In addition to the identification and development of ligands for kinases and other enzymes, determination of the orientation of a molecular scaffold or other binding compound in a binding site allows identification of energetically allowed sites for attachment of the binding molecule to another component. For such sites, any free energy change associated with the presence of the attached component should not destablize the binding of the compound to the kinase to an extent that will disrupt the binding. Preferably, the binding energy with the attachment should be at least 4 kcal/mol., more preferably at least 6, 8, 10, 12, 15, or 20 kcal/mol. Preferably, the presence of the attachment at the particular site reduces binding energy by no more than 3, 4, 5, 8, 10, 12, or 15 kcal/mol.

In many cases, suitable attachment sites will be those that are exposed to solvent when the binding compound is bound in the binding site. In some cases, attachment sites can be used that will result in small displacements of a portion of the enzyme without an excessive energetic cost. Exposed sites can be identified in various ways. For example, exposed sites can be identified using a graphic display or 3-dimensional model. In a grahic display, such as a computer display, an image of a compound bound in a binding site can be visually inspected to reveal atoms or groups on the compound that are exposed to solvent and oriented such that attachment at such atom or group would not preclude binding of the enzyme and binding compound. Energetic costs of attachment can be calculated based on changes or distortions that would be caused by the attachment as well as entropic changes.

Many different types of components can be attached. Persons with skill are familiar with the chemistries used for various attachments. Examples of components that can be attached include, without limitation: solid phase components such as beads, plates, chips, and wells; a direct or indirect label; a linker, which may be a traceless linker; among others. Such linkers can themselves be attached to other components, e.g., to solid phase media, labels, and/or binding moieties.

The binding energy of a compound and the effects on binding energy for attaching the molecule to another component can be calculated approximately using any of a variety of available software or by manual calculation. An example is the following:

Calculations were performed to estimate binding energies of different organic molecules to two Kinases: PIM-1 and CDK2. The organic molecules considered included Staurosporine, identified compounds that bind to PIM-1, and several linkers.

Calculated binding energies between protein-ligand complexes were obtained using the FlexX score (an implementation of the Bohm scoring function) within the Tripos software suite. The form for that equation is shown in Eqn. 1 below:
ΔGbind=ΔGtr+ΔGhb+ΔGion+ΔGlipo+ΔGarom+ΔGrot
where: ΔGtr is a constant term that accounts for the overall loss of rotational and translational entropy of the lignand, ΔGhb accounts for hydrogen bonds formed between the ligand and protein, ΔGion accounts for the ionic interactions between the ligand and protein, ΔGlipo accounts for the lipophilic interaction that corresponds to the protein-ligand contact surface, ΔGarom accounts for interactions between aromatic rings in the protein and ligand, and ΔGrot accounts for the entropic penalty of restricting rotatable bonds in the ligand upon binding.

This method estimates the free energy that a lead compound should have to a target protein for which there is a crystal structure, and it accounts for the entropic penalty of flexible linkers. It can therefore be used to estimate the free energy penalty incurred by attaching linkers to molecules being screened and the binding energy that a lead compound should have in order to overcome the free energy penalty of the linker. The method does not account for solvation and the entropic penalty is likely overestimated for cases where the linker is bound to a solid phase through another binding complex, such as a biotin:streptavidin complex.

Co-crystals were aligned by superimposing residues of PIM-1 with corresponding residues in CDK2. The PIM-1 structure used for these calculations was a co-crystal of PYK2 with a binding compound. The CDK2:Staurosporine co-crystal used was from the Brookhaven database file 1aq1. Hydrogen atoms were added to the proteins and atomic charges were assigned using the AMBER95 parameters within Sybyl. Modifications to the compounds described were made within the Sybyl modeling suite from Tripos.

These calcualtions indicate that the calculated binding energy for compounds that bind strongly to a given target (such as Staurosporine:CDK2) can be lower than −25 kcal/mol, while the calculated binding affinity for a good scaffold or an unoptimized binding compound can be in the range of −15 to −20. The free energy penalty for attachment to a linker such as the ethylene glycol or hexatriene is estimated as typically being in the range of +5 to +15 kcal/mol.

Linkers

Linkers suitable for use in the invention can be of many different types. Linkers can be selected for particular applications based on factors such as linker chemistry compatible for attachment to a binding compound and to another component utilized in the particular application. Additional factors can include, without limitation, linker length, linker stability, and ability to remove the linker at an appropriate time. Exemplary linkers include, but are not limited to, hexyl, hexatrienyl, ethylene glycol, and peptide linkers. Traceless linkers can also be used, e.g., as described in Plunkett, M. J., and Ellman, J. A., (1995), J. Org. Chem., 60:6006.

Typical functional groups, that are utilized to link binding compound(s), include, but not limited to, carboxylic acid, amine, hydroxyl, and thiol. (Examples can be found in Solid-supported combinatorial and parallel synthesis of small molecular weight compound libraries; (1998) Tetrahedron organic chemistry series Vol.17; Pergamon; p85).

Labels

As indicated above, labels can also be attached to a binding compound or to a linker attached to a binding compound. Such attachment may be direct (attached directly to the binding compound) or indirect (attached to a component that is directly or indirectly attached to the binding compound). Such labels allow detection of the compound either directly or indirectly. Attachement of labels can be performed using conventional chemistries. Labels can include, for example, fluorescent labels, radiolabels, light scattering particles, light absorbent particles, magnetic particles, enzymes, and specific binding agents (e.g., biotin or an antibody target moiety).

Solid Phase Media

Additional examples of components that can be attached directly or indirectly to a binding compound include various solid phase media. Similar to attachment of linkers and labels, attachment to solid phase media can be performed using conventional chemistries. Such solid phase media can include, for example, small components such as beads, nanoparticles, and fibers (e.g., in suspension or in a gel or chromatographic matrix). Likewise, solid phase media can include larger objects such as plates, chips, slides, and tubes. In many cases, the binding compound will be attached in only a portion of such an objects, e.g., in a spot or other local element on a generally flat surface or in a well or portion of a well.

Identification of Biological Agents

The posession of structural information about a protein also provides for the identification of useful biological agents, such as epitpose for development of antibodies, identification of mutation sites expected to affect activity, and identification of attachment sites allowing attachment of the protein to materials such as labels, linkers, peptides, and solid phase media.

Antibodies (Abs) finds multiple applications in a variety of areas including biotechnology, medicine and diagnosis, and indeed they are one of the most powerful tools for life science research. Abs directed against protein antigens can recognize either linear or native three-dimensional (3D) epitopes. The obtention of Abs that recognize 3D epitopes require the use of whole native protein (or of a portion that assumes a native conformation) as immunogens. Unfortunately, this not always a choice due to various technical reasons: for example the native protein is just not available, the protein is toxic, or its is desirable to utilize a high density antigen presentation. In such cases, immunization with peptides is the alternative. Of course, Abs generated in this manner will recognize linear epitopes, and they might or might not recognize the source native protein, but yet they will be useful for standard laboratory applications such as western blots. The selection of peptides to use as immunogens can be accomplished by following particular selection rules and/or use of epitope prediction software.

Though methods to predict antigenic peptides are not infallible, there are several rules that can be followed to determine what peptide fragments from a protein are likely to be antigenic. These rules are also dictated to increase the likelihood that an Ab to a particular peptide will recognize the native protein.
1. Antigenic peptides should be located in solvent accessible regions and contain both hydrophobic and hydrophilic residues.
   For proteins of known 3D structure, solvent accessibility can be determined using a variety of programs such as DSSP, NACESS, or WHATIF, among others.
   If the 3D structure is not known, use any of the following web servers to predict accessibilities: PHD, JPRED, PredAcc (c) ACCpro
2. Preferably select peptides lying in long loops connecting Secondary Structure (SS) motifs, avoiding peptides located in helical regions. This will increase the odds that the Ab recognizes the native protein. Such peptides can, for example, be identified from a crystal structure or crystal structure-based homology model.
   For protein with known 3D coordinates, SS can be obtained from the sequence link of the relevant entry at the Brookhaven data bank. The PDBsum server also offer SS analysis of pdb records.
   When no structure is available secondary structure predictions can be obtained from any of the following servers: PHD, JPRED, PSI—PRED, NNSP, etc
3. When possible, choose peptides that are in the N- and C-terminal region of the protein. Because the N- and C-terminal regions of proteins are usually solvent accessible and unstructured, Abs against those regions are also likely to recognize the native protein.
4. For cell surface glycoproteins, eliminate from initial peptides those containing consesus sites for N-glycosilation.
   N-glycosilation sites can be detected using Scanprosite, or NetNGlyc

In addition, several methods based on various physio-chemical properties of experimental determined epitopes (flexibility, hydrophibility, accessibility) have been published for the prediction of antigenic determinants and can be used. The antigenic index and Preditop are example.

Perhaps the simplest method for the prediction of antigenic determinants is that of Kolaskar and Tongaonkar, which is based on the occurrence of amino acid residues in experimentally determined epitopes. (Kolaskar and Tongaonkar (1990) A semi-empirical method for prediction of antigenic determinants on protein antigens. FEBBS Lett. 276(1-2):172-174.) The prediction algorithm works as follows:
1. Calculate the average propensity for each overlapping 7-mer and assign the result to the central residue (i+3) of the 7-mer.
2. Calculate the average for the whole protein.
3. (a) If the average for the whole protein is above 1.0 then all residues having average propensity above 1.0 are potentially antigenic.
3. (b) If the average for the whole protein is below 1.0 then all residues having above the average for the whole protein are potentially antigenic.
4. Find 8-mers where all residues are selected by step 3 above (6-mers in the original paper)

The Kolaskar and Tongaonkar method is also available from the GCG package, and it runs using the command egcg.

Crystal structures also allow identification of residues at which mutation is likely to alter the activity of the protein. Such residues include, for example, residues that interact with susbtrate, conserved active site residues, and residues that are in a region of ordered secondary structure of involved in tertiary interactions. The mutations that are likely to affect activity will vary for different molecular contexts. Mutations in an active site that will affect activity are typically substitutions or deletions that eliminate a charge-charge or hydrogen bonding interaction, or introduce a steric interference. Mutations in secondary structure regions or molecular interaction regions that are likely to affect activity include, for example, substitutions that alter the hydrophobicity/hydrophilicity of a region, or that introduce a sufficient strain in a region near or including the active site so that critical residue(s) in the active site are displaced. Such substitutions and/or deletions and/or insertions are recognized, and the predicted structural and/or energetic effects of mutations can be calculated using conventional software.

IX. Kinase Activity Assays

A number of different assays for kinase activity can be utilized for assaying for active modulators and/or determining specificity of a modulator for a particular kinase or group or kinases. In addition to the assays mentioned below, one of ordinary skill in the art will know of other assays that can be utilized and can modify an assay for a particular application.

An exemplary assay for kinase activity that can be used for PYK2 can be performed according to the following procedure using purified kinase using myelin basic protein (MBP) as substrate. An exemplary assay can use the following materials: MBP (M-1891, Sigma); Kinase buffer (KB=HEPES 50 mM, pH7.2, MgCl₂:MnCl₂ (200 μM:200 μM); ATP (γ-³³P):NEG602H (10 mCi/mL)(Perkin-Elmer); ATP as 100 mM stock in kinase buffer; EDTA as 100 mM stock solution.

Coat scintillation plate suitable for radioactivity counting (e.g., FlashPlate from Perkin-Elmer, such as the SMP200(basic)) with kinase+MBP mix (final 100 ng+300 ng/well) at 90-μL/well in kinase buffer. Add compounds at 1 μL/well from 10 mM stock in DMSO. Positive control wells are added with 1 μL of DMSO. Negative control wells are added with 2 μL of EDTA stock solution. ATP solution (10 μL) is added to each well to provide a final concentration of cold ATP is 2 μM, and 50 nCi ATPγ[³³P]. The plate is shaken briefly, and a count is taken to initiate count (IC) using an apparatus adapted for counting with the plate selected, e.g., Perkin-Elmer Trilux. Store the plate at 37° C. for 4 hrs, then count again to provide final count (FC).

Net 33P incorporation (NI) is calculated as: NI=FC−IC.

The effect of the present of a test compound can then be calculated as the percent of the positive control as: % PC=[(NI−NC)/(PC−NC)]×100, where NC is the net incorporation for the negative control, and PC is the net incorporation for the positive control.

As indicated above, other assays can also be readily used. For example, kinase activity can be measured on standard polystyrene plates, using biotinylated MBP and ATPγ[³³P] and with Streptavidin-coated SPA (scintillation proximity) beads providing the signal.

Additional alternative assays can employ phospho-specific antibodies as detection reagents with biotinylated peptides as substrates for the kinase. This sort of assay can be formatted either in a fluorescence resonance energy transfer (FRET) format, or using an AlphaScreen (amplified luminescent proximity homogeneous assay) format by varying the donor and acceptor reagents that are attached to streptavidin or the phosphor-specific antibody.

X. Organic Synthetic Techniques

The versatility of computer-based modulator design and identification lies in the diversity of structures screened by the computer programs. The computer programs can search databases that contain very large numbers of molecules and can modify modulators already complexed with the enzyme with a wide variety of chemical functional groups. A consequence of this chemical diversity is that a potential modulator of kinase function may take a chemical form that is not predictable. A wide array of organic synthetic techniques exist in the art to meet the challenge of constructing these potential modulators. Many of these organic synthetic methods are described in detail in standard reference sources utilized by those skilled in the art. One example of suh a reference is March, 1994, Advanced Organic Chemistry; Reactions Mechanisms and Structure, New York, McGraw Hill. Thus, the techniques useful to synthesize a potential modulator of kinase function identified by computer-based methods are readily available to those skilled in the art of organic chemical synthesis.

XI. Administration

The methods and compounds will typically be used in therapy for human patients. However, they may also be used to treat similar or identical diseases in other vertebrates such as other primates, sports animals, and pets such as horses, dogs and cats.

Suitable dosage forms, in part, depend upon the use or the route of administration, for example, oral, transdermal, transmucosal, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in Remington's Pharmaceutical Sciences, 18^{th} ed., Mack Publishing Co., Easton, Pa., 1990 (hereby incorporated by reference herein).

Compounds can be formulated as pharmaceutically acceptable salts. Pharmaceutically acceptable salts are non-toxic salts in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methane sulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995. Such salts can be prepared using the appropriate corresponding bases.

Pharmaceutically acceptable salts can be prepared by standard techniques. For example, the free-base form of a compound is dissolved in a suitable solvent, such as an aqueous or aqueous-alcohol in solution containing the appropriate acid and then isolated by evaporating the solution. In another example, a salt is prepared by reacting the free base and acid in an organic solvent.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

Carriers or excipients can be used to produce pharmaceutical compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, or transdermal. Oral administration is preferred. For oral administration, for example, the compounds can be formulated into conventional oral dosage forms such as capsules, tablets, and liquid preparations such as syrups, elixirs, and concentrated drops.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the invention are formulated in sterile liquid solutions, preferably in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The amounts of various compound to be administered can be determined by standard procedures taking into account factors such as the compound IC₅₀, the biological half-life of the compound, the age, size, and weight of the patient, and the disorder associated with the patient. The importance of these and other factors are well known to those of ordinary skill in the art. Generally, a dose will be between about 0.01 and 50 mg/kg, preferably 0.1 and 20 mg/kg of the patient being treated. Multiple doses may be used.

Manipulation of PYK2

As the full-length coding sequence and amino acid sequence of PYK2 is known, cloning, construction of recombinant hPIM-3, production and purification of recombinant protein, introduction of PYK2 into other organisms, and other molecular biological manipulations of PYK2 are readily performed.

Techniques for the manipulation of nucleic acids, such as, e.g., subcloning, labeling probes (e.g., random-primer labeling using Klenow polymerase, nick translation, amplification), sequencing, hybridization and the like are well disclosed in the scientific and patent literature, see, e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, Tijssen, ed. Elsevier, N.Y. (1993).

Nucleic acid sequences can be amplified as necessary for further use using amplification methods, such as PCR, isothermal methods, rolling circle methods, etc., are well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun 1;29(11):E54-E54; Hafner et al., Biotechniques 2001 April; 30(4):852-6, 858, 860 passim; Zhong et al., Biotechniques 2001 April; 30(4):852-6, 858, 860 passim.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be performed by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids. Typically, nucleic acid molecules having a sequence of interest are available from commercial sources and/or from sequence repositories, or can be obtained using PCR from a suitable cDNA or genomic library, e.g., a library from an appropriate tissue. A number of different such libraries are commercially or publicly available.

The nucleic acids can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The nucleic acids of the invention can also be provided in expression vectors and cloning vehicles, e.g., sequences encoding the polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are disclosed, e.g., U.S. Pat. No. 5,426,039. To facilitate cloning of amplified sequences, restriction enzyme sites can be “built into” a PCR primer pair. Vectors may be introduced into a genome or into the cytoplasm or a nucleus of a cell and expressed by a variety of conventional techniques, well described in the scientific and patent literature. See, e.g., Roberts (1987) Nature 328:731; Schneider (1995) Protein Expr. Purif. 6435:10; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

The nucleic acids can be administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous merits of each of the parent vector properties (See e.g., Feng (1997) Nature Biotechnology 15:866-870). Such viral genomes may be modified by recombinant DNA techniques to include the nucleic acids of the invention; and may be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher (1992) J. Virol. 66:2731-2739; Johann (1992) J. Virol. 66:1635-1640). Adeno-associated virus (AAV)-based vectors can be used to transduce cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada (1996) Gene Ther. 3:957-964.

The present invention also relates to fusion proteins, and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif 12:404-414). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well disclosed in the scientific and patent literature, see e.g., Kroll (1993) DNA Cell. Biol. 12:441-53.

The nucleic acids and polypeptides of the invention can be bound to a solid support, e.g., for use in screening and diagnostic methods. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of molecules to a solid support can be direct (i.e., the molecule contacts the solid support) or indirect (a “linker” is bound to the support and the molecule of interest binds to this linker). Molecules can be immobilized either covalently (e.g., utilizing single reactive thiol groups of cysteine residues (see, e.g., Colliuod (1993) Bioconjugate Chem. 4:528-536) or non-covalently but specifically (e.g., via immobilized antibodies (see, e.g., Schuhmann (1991) Adv. Mater. 3:388-391; Lu (1995) Anal. Chem. 67:83-87; the biotin/strepavidin system (see, e.g., Iwane (1997) Biophys. Biochem. Res. Comm. 230:76-80); metal chelating, e.g., Langmuir-Blodgett films (see, e.g., Ng (1995) Langmuir 11:4048-55); metal-chelating self-assembled monolayers (see, e.g., Sigal (1996) Anal. Chem. 68:490-497) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce Chemicals, Rockford, Ill.).

Antibodies can also be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon (1989) Nature 377:525-531 (1989).

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface. In practicing the methods of the invention, any known array and/or method of making and using arrays can be incorporated in whole or in part, or variations thereof, as disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Host Cells and Transformed Cells

The invention also provides a transformed cell comprising a nucleic acid sequence of the invention, e.g., a sequence encoding a polypeptide of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

Vectors may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

For transient expression in mammalian cells, cDNA encoding a polypeptide of interest may be incorporated into a mammalian expression vector, e.g. pcDNA1, which is available commercially from Invitrogen Corporation (San Diego, Calif., U.S.A.; catalogue number V490-20). This is a multifunctional 4.2 kb plasmid vector designed for cDNA expression in eukaryotic systems, and cDNA analysis in prokaryotes, incorporated on the vector are the CMV promoter and enhancer, splice segment and polyadenylation signal, an SV40 and Polyoma virus origin of replication, and M13 origin to rescue single strand DNA for sequencing and mutagenesis, Sp6 and T7 RNA promoters for the production of sense and anti-sense RNA transcripts and a Col E1-like high copy plasmid origin. A polylinker is located appropriately downstream of the CMV promoter (and 3′ of the T7 promoter).

The cDNA insert may be first released from the above phagemid incorporated at appropriate restriction sites in the pcDNA1 polylinker. Sequencing across the junctions may be performed to confirm proper insert orientation in pcDNAI. The resulting plasmid may then be introduced for transient expression into a selected mammalian cell host, for example, the monkey-derived, fibroblast like cells of the COS-1 lineage (available from the American Type Culture Collection, Rockville, Md. as ATCC CRL 1650).

For transient expression of the protein-encoding DNA, for example, COS-1 cells may be transfected with approximately 8 μg DNA per 10⁶ COS cells, by DEAE-mediated DNA transfection and treated with chloroquine according to the procedures described by Sambrook et al, Molecular Cloning: A Laboratory Manual, 1989, Cold Spring Harbor Laboratory Press, Cold Spring Harbor N.Y, pp. 16.30-16.37. An exemplary method is as follows. Briefly, COS-1 cells are plated at a density of 5×10⁶ cells/dish and then grown for 24 hours in FBS-supplemented DMEM/F12 medium. Medium is then removed and cells are washed in PBS and then in medium. A transfection solution containing DEAE dextran (0.4 mg/ml), 100 μM chloroquine, 10% NuSerum, DNA (0.4 mg/ml) in DMEM/F12 medium is then applied on the cells 10 ml volume. After incubation for 3 hours at 37° C., cells are washed in PBS and medium as just described and then shocked for 1 minute with 10% DMSO in DMEM/F12 medium. Cells are allowed to grow for 2-3 days in 10% FBS-supplemented medium, and at the end of incubation dishes are placed on ice, washed with ice cold PBS and then removed by scraping. Cells are then harvested by centrifugation at 1000 rpm for 10 minutes and the cellular pellet is frozen in liquid nitrogen, for subsequent use in protein expression. Northern blot analysis of a thawed aliquot of frozen cells may be used to confirm expression of receptor-encoding cDNA in cells under storage.

In a like manner, stably transfected cell lines can also prepared, for example, using two different cell types as host: CHO K1 and CHO Pro5. To construct these cell lines, cDNA coding for the relevant protein may be incorporated into the mammalian expression vector pRC/CMV (Invitrogen), which enables stable expression. Insertion at this site places the cDNA under the expression control of the cytomegalovirus promoter and upstream of the polyadenylation site and terminator of the bovine growth hormone gene, and into a vector background comprising the neomycin resistance gene (driven by the SV40 early promoter) as selectable marker.

An exemplary protocol to introduce plasmids constructed as described above is as follows. The host CHO cells are first seeded at a density of 5×10⁵ in 10% FBS-supplemented MEM medium. After growth for 24 hours, fresh medium is added to the plates and three hours later, the cells are transfected using the calcium phosphate-DNA co-precipitation procedure (Sambrook et al, supra). Briefly, 3 μg of DNA is mixed and incubated with buffered calcium solution for 10 minutes at room temperature. An equal volume of buffered phosphate solution is added and the suspension is incubated for 15 minutes at room temperature. Next, the incubated suspension is applied to the cells for 4 hours, removed and cells were shocked with medium containing 15% glycerol. Three minutes later, cells are washed with medium and incubated for 24 hours at normal growth conditions. Cells resistant to neomycin are selected in 10% FBS-supplemented alpha-MEM medium containing G418 (1 mg/ml). Individual colonies of G418-resistant cells are isolated about 2-3 weeks later, clonally selected and then propagated for assay purposes.

### EXAMPLES

A number of examples involved in the present invention are described below. In most cases, alternative techniques could also be used. For example, techniques, methods, and other information described in U.S. Pat. No. 5,837,815; U.S. Pat. No. 5,837,524; U.S. Patent Publication 2002/0048782; PCT/US98/02797, WO 98/35056; and McShan et al., Internat. J. Oncology 21:197-205 (2002) can be used in the present invention. Such techniques and information include, without limitation, cloning, culturing, purification, assaying, screening, use of modulators, sequence information, and information concerning biological role of PYK2. Each of these references is incorporated by reference herein in its entirety, including drawings.

### Example 1

### Cloning of PYK2 Kinase Domain

Kinase domain of PYK2 (amino acids 420-691) was amplified by polymerase chain reaction (PCR) using the specific primers 5′-TCCACAGCATATGATTGCCCGTGAAGA TGTGGT-3′ (SEQ ID NO: 5) and 5′-CTCTCGTCGACCTACATGGCAATGTCCTTCTCCA-3′ (SEQ ID NO: 6). The resulting PCR fragment was digested with NdeI and SalI and was ligated into a modified pET15b vector (Novagen) with a cleavable N-terminal hexa-histidine tag (designated pET1S). PYK2 coding sequence has been deposited with GenBank under accession number U33284. A desired PYK2 sequence can be obtained using PCR with a brain (e.g., human brain) cDNA library, such as obtaining kinase domain using the above primers in PCR. The multi-cloning site of the pET15S vector is shown in the following sequence (SEQ ID NO: 7), including the sequence encoding the N-terminal hexa-histadine tag:

[image]

pET15S vector is derived from pET15b vector (Novagen) for bacterial expression to produce the proteins with N-terminal His6. This vector was modified by replacement of NdeI-BamHI fragment to others to create SalI site and stop codon (TAG). Vector size is 5814 bp. Insert can be put using NdeI-SalI site.

The amino acid and nucleic acid sequences for the PYK2 kinase domain utilized are provided in Table 4 (SEQ ID NO: 1 and 3 respectively).

### Example 2

### Expression and Purification of PYK2 Kinase Domain

For protein expression Pyk2 kinase domain was transformed into E. coli strain BL21 (DE3) pLysS and transformants were selected on LB plates containing Kanamycin. Single colonies were grown overnight at 37° C. in 200 ml TB (terrific broth) media. 16×1 L of fresh TB media in 2.8 L flasks were inoculated with 10 ml of overnight culture and grown with constant shaking at 37° C. Once cultures reached an absorbance of 1.0 at 600 nm, 1 mM isopropyl-β-D-thiogalactopyranoside (IPTG) was added and cultures were allowed to grow for a further 12 hrs at 22° C. with constant shaking. Cells were harvested by centrifugation at 7000×g and pellets were frozen in liquid nitrogen and stored at −80° C. until ready for lysis.

The cell pellet was suspended in lysis buffer containing 0.1M Potassium phosphate buffer pH 8.0, 200 mM NaCl, 10% Glycerol, 2 mm PMSF and EDTA free protease inhibitor cocktail tablets (Roche). Cells were lysed using a microfuidizer processor (Microfuidics Corporation) and insoluble cellular debris was removed using centrifugation at 30,000×g. The cleared supernatant was added to Talon resin (Clonetech) and incubated for 4 hrs at 4° C. with constant rocking. The suspension was loaded onto a column and washed with 20 column volumes of lysis buffer plus 10 mM Imadazole. Protein was eluted step wise with addition of lysis buffer plus 200 mM Imadazole pH7.5 and 1 ml fractions collected. Fractions containing PYK2 were pooled, concentrated and loaded onto a Pharmacia HiLoad 26/60 Superdex 200 sizing column (Pharmacia) pre-equilibrated with 20 mM Tris pH7.5, 150 mM NaCl.

Peak fractions were collected and assayed by SDS-PAGE. Fractions containing PYK2 were pooled and diluted in Tris buffer pH 7.5, until 30 mM NaCl was reached. Diluted protein was further subjected to anion exchange chromatography using a Source 15Q (Pharmacia) sepharose column equilibrated with 20 mM Tris pH7.5. Elution was performed using a linear gradient of sodium chloride (0-500 mM). Eluted protein was treated with 2U thrombin per mg protein to remove N-terminal Histidine tag. Following cleavage Pyk2 was re-applied to Source 15Q (Pharmacia) sepharose column equilibrated with 20 mM Tris pH7.5, and eluted using a linear sodium chloride gradient. Purified protein was concentrated to 100 mg/ml and stored at −80° C. until ready for crystallization screening.

### Example 3

### Crystallization of PYK2 Kinase Domain

Crystallization conditions were initially identified in the Hampton Research (Riverside, Calif.) screening kit ₍₁₎. Optimized crystals were grown by vapor diffusion in sitting drop plates with equal volumes of protein solution of 10 mg/ml containing 20 mM Tris-HCl pH 8.0, 150 mM NaCl, 14 mM BME, 1 mM DTT and reservoir solution containing 8% polyethylene glycol (PEG) 8000, 0.2M Sodium Acetate, 0.1 M Cacodylate pH 6.5, 20% Glycerol). Blades of crystals grew overnight at 4° C. Microseeding was used to produce larger, single crystals, the largest crystal being around 0.3 mm×0.05 mm×0.02 mm.

### Example 4

### Diffraction Analysis of PYK2

Synchrotron X-ray data for Pyk2 was collected at beamline 8.3.1 of the Advanced Light Source (ALS, Lawrence Berkeley National Laboratory, Berkeley) on a Quantum 210 charge-coupled device detector (λ=1.10 Å). The mother liquor from the reservoir was used as cryo-protectant for the crystal. Detector distance was 110 mm and exposure time was 10 s per frame. 200 frames were collected with 0.5° oscillation over a wedge of 100°. The quality and resolution limits of the diffraction pattern were considerably improved by annealing the crystal. The crystal was briefly allowed to warm up for 10 seconds by shutting off the Nitrogen cryo stream and refrozen by resuming cooling with the cryo stream. Crystals of PYK2 diffracted to a resolution limit of 1.45 Å with cell dimensions of a=37 Å, b=47 Å, c=81 Å, α=90°, β=92°, γ=90°. The data were processed using Mosflm ( ) and scaled and reduced with Scala ( ) in CCP40 in space group P2. The data processing process was driven by the ELVES automation scripts (J. M. Holton, unpublished data). An inspection of the 0K0 zone indicated that all odd (2n+1) reflections were very weak compared with the even reflections, suggesting the space group to be P2₁.

PYK2 Structure Determination and Refinement

The initial phases for the dataset were obtained by molecular replacement. A homology model of the protein Pyk2 was generated using the LCK kinase structure (PDBID: 1qpc) as a template. This model was trimmed by excising all loops before being used in molecular replacement program EPMR ( ), which resulted in a solution with CC=0.372. The molecular replacement solution phases were improved by the program Arp-Warp ( ). The resultant model was further improved by manual model building and extension in O ( ) and refinement with CNX ( ) and Refinac5 ( ) in CCP4. The cycle of model building and refinement continued till the model was complete and refinement converged to the R/Rfree of 20.83/26.94%. The geometric analysis of the model was performed by PROCHECK ( ) which indicated the structure to have excellent geometry.

Data collection and refinement statistics for PYK2 kinase domain crystal, and for PYK2 kinase domain/binding compound cocrystal are summarized in the following table:

Data Collection and Refinement Statistics

| | Pyk2 (APO) | Pyk2 + AMPPNP |
|---|---|---|
| Crystal Parameters | | |
| Space Group | P2₁ | P2₁ |
| Unit Cell (Å) | a = 37.17, b = 46.97, | a = 37.32, b = 46.98, |
| | c = 80.36, [symbol] = 92.63 | c = 81.11, [symbol] = 92.83 |
| Number of | 1 | 1 |
| molecules/AU | | |
| V_{M} (Å³/Dalton) | 2.4 | 2.4 |
| Solvent content (%) | 48 | 48 |
| Data Collection and | | |
| Processing | | |
| Resolution (Å) | 1.45 | 1.80 |
| Wavelength (Å) | 1.1 | 1.1 |
| Unique reflections | 47843 | 26149 |
| Redundancy (last shell*) | 2.0 (1.8) | 4.0 (2.9) |
| Completeness (last | 97.5 (88.9) | 99.8 (97.8) |
| shell) (%) | | |
| I/ [symbol] (last shell) | 10.9 (1.3) | 12.0 (2.3) |
| R_{sym} (last shell) | 0.043 (0.487) | 0.063 (0.459) |
| *Last shell (Å) | 1.49-1.45 | 1.85-1.80 |
| Refinement | | |
| R_{work}/R_{free} (%) | 16.93/20.68 | 18.62/22.81 |
| Number of Atoms | 2583 | 2507 |
| Rmsd from ideal | 0.012 (bond distance), | 0.010 (bond distance), |
| geometry | 1.434 (bond angle) | 1.372 (bond angle) |
| SigmaA coordinate error | 0.16 Å | 0.14 Å |
| | (for 5.0-1.45 Å) | (for 5.0-1.80 Å) |
| Average B-factors | 19.3 | 20.5 |
| (Å²) | | |
| Protein atoms | 16.4 | 19.0 |
| Waters | 37.6 | 34.3 |
| Ligand | — | 44.41 |

The model of Pyk2 contains 273 amino acids (spanning the PYK2 sequence 420-691 with one residue from the cloning vector) and 180 water molecules. The Pyk2 structure adopts the standard kinase fold consisting of an N-terminal β-sheet domain and a C-terminal α-helical domain linked by a 5 residue linker. The linker segment contains the canonical H-bond acceptor/donor residues E503 and Y505 that would normally interact with the adenosine ring of ATP. In the apo structure these residues make H-bonds with water molecules.

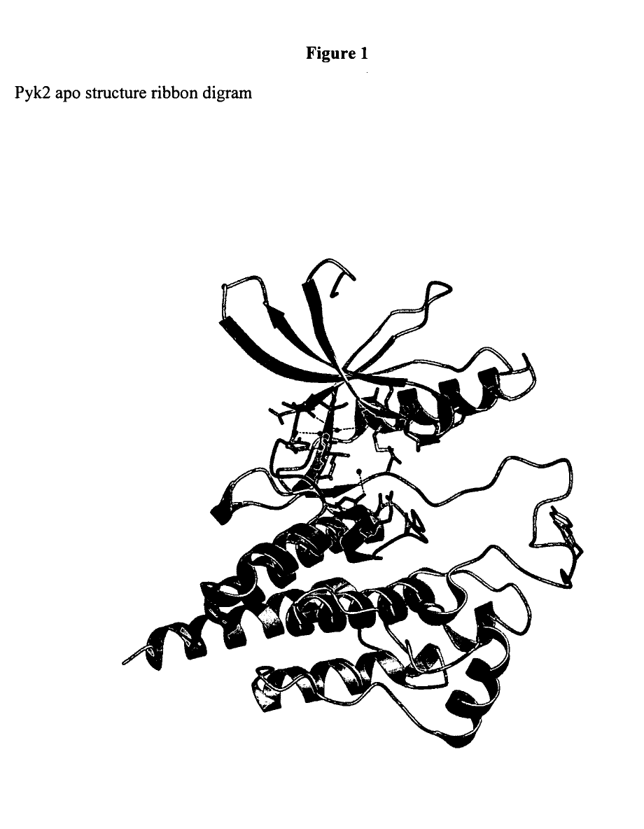

A ribbon diagram of the PYK2 active site is shown in FIG. 1. Atomic coordinates for the apo protein are provided in Table 1, while atomic coordinates for a PYK2 co-crystallized with a binding compound (AMPPNP) are provided in Table 2.

Active Loop Conformation

In many protein kinases, the activation loop, or A-loop, plays an important role in regulating the kinase activity. In active kinases, the A-loops adopt a highly similar conformation characterized by the formation of three small β-sheet moieties: two with the main body of the protein (the beginning of the catalytic or C-loop and the αEF/αF loop, respectively), and one with the substrate peptide. In contrast, the inactive conformation of A-loop differs markedly from protein to protein, albeit having the similar effect of blocking ATP binding, substrate-binding, or both. In comparison with the active insulin receptor (INSR) and IGFR1 kinase domain strutures, the A-loop in the solved Pyk2 structure is clearly in an inactive conformation. The loop is stabilized by a unique set of intra- and inter-loop interactions that differentiate it from all known A-loop structures.

The A-loop in our Pyk2 structure starts to deviate from the standard active conformation at the DFG motif (for comparison, we modeled the active A-loop conformation of Pyk2 based on the IGFR1 structure). The first two residues of the DFG motif (D⁵⁶⁷ and F⁵⁶⁸) have similar orientations as their counterparts in the active A-loop form, with D⁵⁶⁷ interacting with K⁴⁵⁷ (β3) and F⁵⁶⁸ locked in a hydrophobic pocket sandwiched by two residues (I⁴⁷⁷ and M⁴⁷⁸) from αC. However, the third residue in the motif, G⁵⁶⁹, adopts a completely different conformation, resulting in the formation of a hydrogen bond beween G⁵⁶⁷:NH and H⁵⁴⁷:CO. This hydrogen bond forces the A-loop to a different path that precludes it from forming a β-sheet with C-loop. A similar hydrogen bond has also been observed in two other tyrosine kinases: HCK (1qcf) and SRC (1fmk).

There are multiple interactions that help to stabilize the A-loop in its observed conformation. Most of them involve a unique sequence moiety of Pyk2. Among the tyrosine kinases of known structure, Pyk2 contains a unique ED repeat (E⁵⁷⁵-D⁵⁷⁸) in the A-loop. In the Pyk2 structure, E⁵⁷⁵ is exposed to solvent, whereas D⁵⁷⁶ initiates a tight β -turn. Beside providing the canonical β-turn backbone hydrogen bond between D⁵⁷⁶:CO—Y⁵⁷⁹:NH, the side chain of D⁵⁷⁶ also interacts with D⁵⁷⁸:NH. The β-turn region of A-loop is held to the αEF/αF loop by two side-chain-backbone hydrogen bonds: one between E⁵⁷⁷:CO—R⁶⁰⁰:Ne and the other between K⁵⁸¹:NZ-N⁵⁹⁸:CO. The side chain of E⁵⁷⁷ interacts with the end of the activation loop via two hydrogen bonds, one with T⁵⁸⁵ (OG) and the other with R⁵⁸⁶ (NH). The most interesting feature of the Pyk2 A-loop is the salt bridge formed between D⁵⁸⁸ and R⁵⁴⁷ from the C-loop (the distances between the two OD and two NH atoms are 2.9 Å). Neither of the two tyrosines Y⁵⁷⁹ and Y⁵⁸⁰ is phosphorylated in our structure. Y⁵⁷⁹ is exposed to solvent, whereas Y⁵⁸⁰ binds to the hydrophobic portions of the E⁵⁷⁵ and E⁵⁷⁷ side chains.

Because FAK does not have the second ED, the conformation of the A-loop in an inactive FAK is expected to be different.

Implications for Substrate Binding and Autophosphorylation

An important event in the enzymatic activation of FAK/Pyk2 is the autophosphorylation of a tyrosine residue before the catalytic domain (Y402). The phosphorylated Y402 provides the binding site for Src and other related kinases and facilitates Src-dependent phosphorylation of other tyrosine residues on Pyk2 including Y579 and Y580. It is not clear how autophosphorylation could occur before Y579 and Y580 are phosphorylated.

To test whether Y402 can reach the substrate binding site, we modeled the 7 residue peptide D⁴⁰⁰IYAEIPD⁴⁰⁷ containing Y⁴⁰² into the substrate binding site based on the cocrystal structure of IGFR1 kinase domain with its substrate peptide. In our protein construct, the Pyk2 insert starts at 1420. There are four residues (GSHM) N-terminal to 1420 left by the His-tag used, of those only M419 is visible. We then modeled the 11 residues that link D419 to M407. The model shows that, in order to reach the substrate binding site, the N-terminal region has to transverse along the back of aC. The link would also fix the A-loop in the active conformation. This may provide the mechanism that the protein used to autophosphorylate Y402. Once Y402 is phosphorylated, the N-terminus is then released and subject to SH2 binding. The A-loop also becomes flexible and accessible to Src.

Because the residues surrounding the P+1 and P+3 binding pocket are mostly hydrophobic in tyrosine kinases, substrate P+1 and P+3 sites are mostly hydrophobic residues. The residue that might interact with P+2 varies. Acidic and other polar site chains might be preferred because of the nearby residue R586. The P−1 site is an acidic residue in INSR and IGFR1. The residue for interacting with P−1 is Arg; this residue is changed to Gly in Pyk2, leaving the space largely hydrophobic. The autophosphorylation site sequence in Pyk2, IYAEIPD, and the sequences of several other known Pyk2 phosphorylation sites fit well the substrate selectivity profile of Pyk2.

### Example 5

### PYK2 Binding Assays

Binding assays can be performed in a variety of ways, including a variety of ways known in the art. For example, competitive binding to PYK2 can be measured on Nickel-FlashPlates, using His-tagged PYK2 (˜100 ng) and ATPγ[³⁵S] (˜10 nCi). As compound is added, the signal decreases, since less ATPγ[³⁵S] is bound to PYK2 which is proximal to the scintillant in the FlashPlate. The binding assay can be performed by the addition of compound (10 μl; 20 mM) to PYK2 protein or kinase domain (90 10 μl) followed by the addition of ATPγ[³⁵S] and incubating for 1 hr at 37° C. The radioactivity is measured through scintillation counting in Trilus (Perkin-Elmer).

Alternatively, any method which can measure binding of a ligand to the ATP-binding site can be used. For example, a fluorescent ligand can be used. When bound to PYK2, the emitted fluorescence is polarized. Once displaced by inhibitor binding, the polarization decreases.

Determination of IC50 for compounds by competitive binding assays. (Note that K_{I} is the dissociation constant for inhibitor binding; K_{D} is the dissociation constant for substrate binding.) For this system, the IC50, inhibitor binding constant and substrate binding constant can be interrelated according to the following formula:

When using radiolabeled substrate

$${K}_{I} = \frac{IC 50}{1 + [ {L}^{*} ] \text{/} {K}_{D}} ,$$

the IC50˜K_{I} when there is a small amount of labeled substrate.

### Example 6

### PYK2 Activity Assay

As an exemplary kinase assay, the kinase activity of PYK2 was measured in AlphaScreening (Packard BioScience). The kinase buffer (HMNB) contains HEPES 50 mM at pH7.2, Mg/Mn 5 mM each, NP-40 0.1%, and BSA at final 50 ug/ml. AlphaScreening is conducted as described by the manufacturer. In brief, the kinase reaction is performed in 384-well plate in 25 ul volume. The substrate is biotin-(E4Y)₃ at final concentration of 1 nM. The final concentration of ATP is 10 uM. For compound testing the final DMSO concentration is 1%. The reaction is incubated in 31° C. for 1 hour.

The Pyk2 kinase domain residues 419 to 691 is an active kinase in AlphaScreen. At a concentration of 8 ng/well in 384-well plate, PYK2 shows a Kd of 7.34 uM, which is in general agreement with most protein kinases (Table 5). Inhibition by ATP analogs was tested with Pyk2 at 8 ng/well and ATP at 10 uM. The data is shown in Table 5. The affinity of ATP-g-S and ADP with Pyk2 is at 14 uM. Adenosine and AMP-PCP have little effect on PYK2 in the concentration tested.

### Example 9

### Synthesis of the Compounds of Formula I:

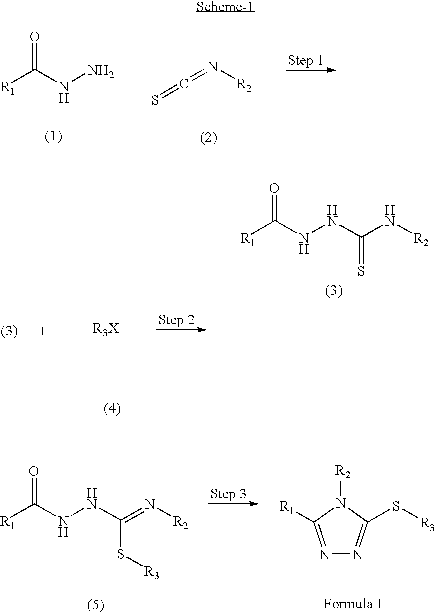

The triazole derivatives, represented by Formula I, can be prepared as shown in Scheme-1.

Step-1 Preparation of Formula (3)

The compound of formula (3) is prepared conventionally by reaction of a compound of formula (1), where R₁=alkyl, aryl, heteroaryl (e.g. m-toluic hydrazide), with an isothiocyanate of formula (2), in a basic solvent (e.g. pyridine), typically heated near 65° C. for 2-6 hours.

Step-2 Preparation of Formula (5)

The compound of formula (5) is prepared conventionally by reaction of a compound of formula (3) with an alkylating agent of formula (4)(e.g. methyl iodide), in an inert solvent (e.g. THF) at room temperature for 24-48 hours.

Step-3 Preparation of Formula I

The compound of Formula I is prepared by dissolving a compound of formula (5) in POCl3 and heated near 80° C. for 8-12 hours. When the reaction is substantially complete, the product of Formula I is isolated by conventional means (e.g. reverse phase HPLC). Smith, et. al., J. Comb. Chem., 1999, 1, 368-370; and references therein.

### Example 10

### Site-Directed Mutagenesis of PYK2 kinase

Mutagenesis of PYK2 kinase can be carried out according to the following procedure as described in Molecular Biology: Current Innovations and Future Trends. Eds. A. M. Griffin and H. G. Griffin. (1995) ISBN 1-898486-01-8, Horizon Scientific Press, PO Box 1, Wymondham, Norfolk, U.K., among others.

In vitro site-directed mutagenesis is an invaluable technique for studying protein structure-function relationships, gene expression and vector modification. Several methods have appeared in the literature, but many of these methods require single-stranded DNA as the template. The reason for this, historically, has been the need for separating the complementary strands to prevent reannealing. Use of PCR in site-directed mutagenesis accomplishes strand separation by using a denaturing step to separate the complementing strands and allowing efficient polymerization of the PCR primers. PCR site-directed methods thus allow site-specific mutations to be incorporated in virtually any double-stranded plasmid; eliminating the need for M13-based vectors or single-stranded rescue.

It is often desirable to reduce the number of cycles during PCR when performing PCR-based site-directed mutagenesis to prevent clonal expansion of any (undesired) second-site mutations. Limited cycling which would result in reduced product yield, is offset by increasing the starting template concentration. A selection is used to reduce the number of parental molecules coming through the reaction. Also, in order to use a single PCR primer set, it is desirable to optimize the long PCR method. Further, because of the extendase activity of some thermostable polymerases it is often necessary to incorporate an end-polishing step into the procedure prior to end-to-end ligation of the PCR-generated product containing the incorporated mutations in one or both PCR primers.

The following protocol provides a facile method for site-directed mutagenesis and accomplishes the above desired features by the incorporation of the following steps:
(i) increasing template concentration approximately 1000-fold over conventional PCR conditions; (ii) reducing the number of cycles from 25-30 to 5-10; (iii) adding the restriction endonuclease DpnI (recognition target sequence: 5-Gm6ATC-3, where the A residue is methylated) to select against parental DNA (note: DNA isolated from almost all common strains of E. coli is Dam-methylated at the sequence 5-GATC-3); (iv) using Taq Extender in the PCR mix for increased reliability for PCR to 10 kb; (v) using Pfu DNA polymerase to polish the ends of the PCR product, and (vi) efficient intramolecular ligation in the presence of T4 DNA ligase.

Plasmid template DNA (approximately 0.5 pmole) is added to a PCR cocktail containing, in 25 ul of 1× mutagenesis buffer: (20 mM Tris HCl, pH 7.5; 8 mM MgCl2; 40 ug/ml BSA); 12-20 pmole of each primer (one of which must contain a 5-prime phosphate), 250 uM each dNTP, 2.5 U Taq DNA polymerase, 2.5 U of Taq Extender (Stratagene).

The PCR cycling parameters are 1 cycle of: 4 min at 94 C, 2 min at 50 C and 2 min at 72 C; followed by 5-10 cycles of 1 min at 94 C, 2 min at 54 C and 1 min at 72 C (step 1).

The parental template DNA and the linear, mutagenesis-primer incorporating newly synthesized DNA are treated with DpnI (10 U) and Pfu DNA polymerase (2.5U). This results in the DpnI digestion of the in vivo methylated parental template and hybrid DNA and the removal, by Pfu DNA polymerase, of the Taq DNA polymerase-extended base(s) on the linear PCR product.

The reaction is incubated at 37 C for 30 min and then transferred to 72 C for an additional 30 min (step 2).

Mutagenesis buffer (1×, 115 ul, containing 0.5 mM ATP) is added to the DpnI-digested, Pfu DNA polymerase-polished PCR products.

The solution is mixed and 10 ul is removed to a new microfuge tube and T4 DNA ligase (2-4 U) added.

The ligation is incubated for greater than 60 min at 37 C (step 3).

The treated solution is transformed into competent E. coli (step 4).

In addition to the PCT-based site-directed mutagenesis described above, other methods are available. Examples include those described in Kunkel (1985) Proc. Natl. Acad. Sci. 82:488-492; Eckstein et al. (1985) Nucl. Acids Res. 13:8764-8785; and using the GeneEditor™ Site-Directed Mutageneis Sytem from Promega.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to crystallization or co-crystallization conditions for PYK2 proteins and/or various kinase domain sequences can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

TABLE 1
REMARK Written by DEALPDB Version 1.13 (06/02)
REMARK Fri Nov 8 15:01:36 2002

| | | |
|---|---|---|
| HEADER | ---- | XX-XXX-XX xxxx |

| COMPND | --- | | |
|---|---|---|---|
| REMARK | 3 | | |
| REMARK | 3 | REFINEMENT. | |
| REMARK | 3 | PROGRAM : REFMAC 5.1.25 | |
| REMARK | 3 | AUTHORS : MURSHUDOV, VAGIN, DODSON | |
| REMARK | 3 | | |
| REMARK | 3 | REFINEMENT TARGET : MAXIMUM LIKELIHOOD | |
| REMARK | 3 | | |
| REMARK | 3 | DATA USED IN REFINEMENT. | |
| REMARK | 3 | RESOLUTION RANGE HIGH (ANGSTROMS) | : 1.45 |
| REMARK | 3 | RESOLUTION RANGE LOW (ANGSTROMS) | : 79.06 |
| REMARK | 3 | DATA CUTOFF (SIGMA(F)) | : NONE |
| REMARK | 3 | COMPLETENESS FOR RANGE (%) | : 97.02 |
| REMARK | 3 | NUMBER OF REFLECTIONS | : 45396 |
| REMARK | 3 | | |

| REMARK | 3 | FIT TO DATA USED IN REFINEMENT. | |
|---|---|---|---|
| REMARK | 3 | CROSS-VALIDATION METHOD | : THROUGHOUT |
| REMARK | 3 | FREE R VALUE TEST SET SELECTION | : RANDOM |
| REMARK | 3 | R VALUE (WORKING + TEST SET) | : 0.17122 |
| REMARK | 3 | R VALUE (WORKING SET) | : 0.16934 |
| REMARK | 3 | FREE R VALUE | : 0.20676 |
| REMARK | 3 | FREE R VALUE TEST SET SIZE (%) | : 5.0 |
| REMARK | 3 | FREE R VALUE TEST SET COUNT | : 2407 |
| REMARK | 3 | | |

| REMARK | 3 | FIT IN THE HIGHEST RESOLUTION BIN. | |
|---|---|---|---|
| REMARK | 3 | TOTAL NUMBER OF BINS USED | : 20 |
| REMARK | 3 | BIN RESOLUTION RANGE HIGH | : 1.450 |
| REMARK | 3 | BIN RESOLUTION RANGE LOW | : 1.488 |
| REMARK | 3 | REFLECTION IN BIN (WORKING SET) | : 3077 |
| REMARK | 3 | BIN R VALUE (WORKING SET) | : 0.283 |
| REMARK | 3 | BIN FREE R VALUE SET COUNT | : 151 |
| REMARK | 3 | BIN FREE R VALUE | : 0.287 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS : 2583 | |
| REMARK | 3 | | |
| REMARK | 3 | B VALUES. | |
| REMARK | 3 | FROM WILSON PLOT (A**2) | : NULL |
| REMARK | 3 | MEAN B VALUE (OVERALL, A**2) | : 15.129 |

| REMARK | 3 | OVERALL ANISOTROPIC B VALUE. | | |
|---|---|---|---|---|
| REMARK | 3 | B11 (A**2) : −0.45 | | |
| REMARK | 3 | B22 (A**2) : 0.51 | | |
| REMARK | 3 | B33 (A**2) : −0.07 | | |
| REMARK | 3 | B12 (A**2) : 0.00 | | |
| REMARK | 3 | B13 (A**2) : −0.23 | | |
| REMARK | 3 | B23 (A**2) : 0.00 | | |
| REMARK | 3 | | | |
| REMARK | 3 | ESTIMATED OVERALL COORDINATE ERROR. | | |
| REMARK | 3 | ESU BASED ON R VALUE | (A): | 0.083 |
| REMARK | 3 | ESU BASED ON FREE R VALUE | (A): | 0.073 |
| REMARK | 3 | ESU BASED ON MAXIMUM LIKELIHOOD | (A): | 0.046 |
| REMARK | 3 | ESU FOR B VALUES BASED ON MAXIMUM LIKELIHOOD | (A**2): | 1.218 |
| REMARK | 3 | | | |

| REMARK | 3 | CORRELATION COEFFICIENTS. | | | | |
|---|---|---|---|---|---|---|
| REMARK | 3 | CORRELATION COEFFICIENT FO-FC : 0.966 | | | | |
| REMARK | 3 | CORRELATION COEFFICIENT FO-FC FREE : 0.949 | | | | |
| REMARK | 3 | | | | | |
| REMARK | 3 | RMS DEVIATIONS FROM IDEAL VALUES | | COUNT | RMS | WEIGHT |
| REMARK | 3 | BOND LENGTHS REFINED ATOMS | (A): | 2278 ; | 0.012 ; | 0.022 |
| REMARK | 3 | BOND LENGTHS OTHERS | (A): | 2095 ; | 0.002 ; | 0.020 |
| REMARK | 3 | BOND ANGLES REFINED ATOMS | (DEGREES): | 3079 ; | 1.434 ; | 1.970 |
| REMARK | 3 | BOND ANGLES OTHERS | (DEGREES): | 4880 ; | 1.216 ; | 3.000 |
| REMARK | 3 | TORSION ANGLES, PERIOD 1 | (DEGREES): | 271 ; | 5.456 ; | 5.000 |
| REMARK | 3 | CHIRAL-CENTER RESTRAINTS | (A**3): | 339 ; | 0.083 ; | 0.200 |
| REMARK | 3 | GENERAL PLANES REFINED ATOMS | (A): | 2465 ; | 0.009 ; | 0.020 |
| REMARK | 3 | GENERAL PLANES OTHERS | (A): | 462 ; | 0.011 ; | 0.020 |
| REMARK | 3 | NON-BONDED CONTACTS REFINED ATOMS | (A): | 517 ; | 0.238 ; | 0.200 |
| REMARK | 3 | NON-BONDED CONTACTS OTHERS | (A): | 2522 ; | 0.234 ; | 0.200 |
| REMARK | 3 | NON-BONDED TORSION OTHERS | (A): | 1336 ; | 0.088 ; | 0.200 |
| REMARK | 3 | H-BOND (X...Y) REFINED ATOMS | (A): | 241 ; | 0.163 ; | 0.200 |
| REMARK | 3 | SYMMETRY VDW REFINED ATOMS | (A): | 16 ; | 0.108 ; | 0.200 |
| REMARK | 3 | SYMMETRY VDW OTHERS | (A): | 93 ; | 0.279 ; | 0.200 |
| REMARK | 3 | SYMMETRY H-BOND REFINED ATOMS | (A): | 23 ; | 0.131 ; | 0.200 |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2): | 1362 ; | 1.094 ; | 1.500 |
| REMARK | 3 | MAIN-CHAIN ANGLE REFINED ATOMS | (A**2): | 2217 ; | 1.859 ; | 2.000 |
| REMARK | 3 | SIDE-CHAIN BOND REFINED ATOMS | (A**2): | 916 ; | 2.488 ; | 3.000 |
| REMARK | 3 | SIDE-CHAIN ANGLE REFINED ATOMS | (A**2): | 862 ; | 3.822 ; | 4.500 |
| REMARK | 3 | | | | | |
| REMARK | 3 | ANISOTROPIC THERMAL FACTOR RESTRAINTS. | | COUNT | RMS | WEIGHT |
| REMARK | 3 | RIGID-BOND RESTRAINTS | (A**2): | 2278 ; | 1.321 ; | 2.000 |
| REMARK | 3 | SPHERICITY; BONDED ATOMS | (A**2): | 2226 ; | 1.814 ; | 2.000 |
| REMARK | 3 | | | | | |

| REMARK | 3 | NCS RESTRAINTS STATISTICS | |
|---|---|---|---|
| REMARK | 3 | NUMBER OF NCS GROUPS : NULL | |
| REMARK | 3 | | |
| REMARK | 3 | | |
| REMARK | 3 | TLS DETAILS | |
| REMARK | 3 | NUMBER OF TLS GROUPS : 1 | |
| REMARK | 3 | | |
| REMARK | 3 | TLS GROUP : 1 | |
| REMARK | 3 | NUMBER OF COMPONENTS GROUP : 1 | |
| REMARK | 3 | COMPONENTS C SSSEQI TO C SSSEQI | |
| REMARK | 3 | RESIDUE RANGE : A 419 A 691 | |
| REMARK | 3 | ORIGIN FOR THE GROUP (A) : 7.0590 1.6770 18.9230 | |
| REMARK | 3 | T TENSOR | |
| REMARK | 3 | T11: 0.0106 T22: 0.0198 | |
| REMARK | 3 | T33: 0.0169 T12: −0.0142 | |
| REMARK | 3 | T13: −0.0005 T23: 0.0042 | |
| REMARK | 3 | L TENSOR | |
| REMARK | 3 | L11: 0.7756 L22: 0.7085 | |
| REMARK | 3 | L33: 0.5853 L12: −0.2205 | |
| REMARK | 3 | L13: 0.1565 L23: −0.0117 | |
| REMARK | 3 | S TENSOR | |
| REMARK | 3 | S11: −0.0307 S12: −0.0104 S13: 0.0730 | |
| REMARK | 3 | S21: 0.0204 S22: 0.0478 S23: −0.0005 | |
| REMARK | 3 | S31: −0.0401 S32: 0.0386 S33: −0.0171 | |
| REMARK | 3 | | |
| REMARK | 3 | | |
| REMARK | 3 | BULK SOLVENT MODELLING. | |
| REMARK | 3 | METHOD USED : BABINET MODEL WITH MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |

| | |
|---|---|
| CRYST1 | 37.173 46.970 80.360 90.00 92.63 90.00 P 1 21 1 |

| | | | | |
|---|---|---|---|---|
| SCALE1 | 0.026901 | 0.000000 | 0.001235 | 0.00000 |
| SCALE2 | 0.000000 | 0.021290 | 0.000000 | 0.00000 |
| SCALE3 | 0.000000 | 0.000000 | 0.012457 | 0.00000 |

| | | | | | | | | | | | | | |
|---|---|---|---|---|---|---|---|---|---|---|---|---|---|
| ATOM | 1 | N | MET | A | 419 | −17.798 | 13.824 | 26.716 | 1.00 | 37.08 | | A | N |
| ANISOU | 1 | N | MET | A | 419 | 4698 | 4704 | 4686 | 2 | −13 | 12 | A | N |
| ATOM | 3 | CA | MET | A | 419 | −17.141 | 14.629 | 25.645 | 1.00 | 36.94 | | A | C |
| ANISOU | 3 | CA | MET | A | 419 | 4672 | 4681 | 4680 | −19 | −7 | −4 | A | C |
| ATOM | 5 | CB | MET | A | 419 | −18.186 | 15.173 | 24.668 | 1.00 | 37.63 | | A | C |
| ANISOU | 5 | CB | MET | A | 419 | 4763 | 4778 | 4757 | 9 | −9 | 24 | A | C |
| ATOM | 8 | CG | MET | A | 419 | −19.078 | 14.098 | 24.049 | 1.00 | 39.47 | | A | C |
| ANISOU | 8 | CG | MET | A | 419 | 4983 | 5017 | 4994 | −61 | −50 | 8 | A | C |
| ATOM | 11 | SD | MET | A | 419 | −18.149 | 12.778 | 23.218 | 1.00 | 42.55 | | A | S |
| ANISOU | 11 | SD | MET | A | 419 | 5414 | 5343 | 5409 | 11 | 26 | −34 | A | S |
| ATOM | 12 | CE | MET | A | 419 | −17.963 | 11.571 | 24.548 | 1.00 | 42.75 | | A | C |
| ANISOU | 12 | CE | MET | A | 419 | 5417 | 5401 | 5423 | −17 | −19 | 12 | A | C |
| ATOM | 16 | C | MET | A | 419 | −16.343 | 15.776 | 26.257 | 1.00 | 35.96 | | A | C |
| ANISOU | 16 | C | MET | A | 419 | 4538 | 4570 | 4553 | −2 | 21 | 4 | A | C |
| ATOM | 17 | O | MET | A | 419 | −16.823 | 16.469 | 27.161 | 1.00 | 36.07 | | A | O |
| ANISOU | 17 | O | MET | A | 419 | 4561 | 4581 | 4561 | −5 | 15 | −19 | A | O |
| ATOM | 20 | N | ILE | A | 420 | −15.136 | 15.980 | 25.730 | 1.00 | 34.59 | | A | N |
| ANISOU | 20 | N | ILE | A | 420 | 4374 | 4378 | 4388 | 7 | −11 | −22 | A | N |
| ATOM | 22 | CA | ILE | A | 420 | −14.140 | 16.850 | 26.347 | 1.00 | 33.36 | | A | C |
| ANISOU | 22 | CA | ILE | A | 420 | 4229 | 4219 | 4225 | 20 | 2 | 7 | A | C |
| ATOM | 24 | CB | ILE | A | 420 | −12.741 | 16.141 | 26.376 | 1.00 | 33.70 | | A | C |
| ANISOU | 24 | CB | ILE | A | 420 | 4255 | 4286 | 4261 | 15 | 4 | −12 | A | C |
| ATOM | 26 | CG1 | ILE | A | 420 | −11.770 | 16.911 | 27.282 | 1.00 | 34.11 | | A | C |
| ANISOU | 26 | CG1 | ILE | A | 420 | 4297 | 4329 | 4332 | 12 | −13 | 0 | A | C |
| ATOM | 29 | CD1 | ILE | A | 420 | −10.797 | 17.813 | 26.577 | 1.00 | 34.87 | | A | C |
| ANISOU | 29 | CD1 | ILE | A | 420 | 4423 | 4406 | 4417 | −5 | 13 | 28 | A | C |
| ATOM | 33 | CG2 | ILE | A | 420 | −12.180 | 15.885 | 24.948 | 1.00 | 34.10 | | A | C |
| ANISOU | 33 | CG2 | ILE | A | 420 | 4312 | 4331 | 4310 | 1 | 23 | 6 | A | C |
| ATOM | 37 | C | ILE | A | 420 | −14.057 | 18.233 | 25.694 | 1.00 | 31.72 | | A | C |
| ANISOU | 37 | C | ILE | A | 420 | 4002 | 4045 | 4003 | 25 | −4 | −28 | A | C |
| ATOM | 38 | O | ILE | A | 420 | −13.902 | 18.365 | 24.480 | 1.00 | 32.15 | | A | O |
| ANISOU | 38 | O | ILE | A | 420 | 4033 | 4132 | 4050 | 36 | −20 | 13 | A | O |
| ATOM | 39 | N | ALA | A | 421 | −14.152 | 19.265 | 26.522 | 1.00 | 29.54 | | A | N |
| ANISOU | 39 | N | ALA | A | 421 | 3728 | 3740 | 3757 | 13 | −21 | 49 | A | N |
| ATOM | 41 | CA | ALA | A | 421 | −14.110 | 20.642 | 26.055 | 1.00 | 27.85 | | A | C |
| ANISOU | 41 | CA | ALA | A | 421 | 3497 | 3550 | 3531 | −15 | −12 | 6 | A | C |
| ATOM | 43 | CB | ALA | A | 421 | −15.025 | 21.514 | 26.899 | 1.00 | 27.73 | | A | C |
| ANISOU | 43 | CB | ALA | A | 421 | 3508 | 3516 | 3512 | 2 | −4 | 23 | A | C |
| ATOM | 47 | C | ALA | A | 421 | −12.683 | 21.138 | 26.141 | 1.00 | 26.04 | | A | C |
| ANISOU | 47 | C | ALA | A | 421 | 3309 | 3286 | 3297 | 18 | 0 | 47 | A | C |
| ATOM | 48 | O | ALA | A | 421 | −11.905 | 20.642 | 26.948 | 1.00 | 25.36 | | A | O |
| ANISOU | 48 | O | ALA | A | 421 | 3159 | 3228 | 3247 | 37 | −3 | 6 | A | O |
| ATOM | 49 | N | ARG | A | 422 | −12.355 | 22.138 | 25.331 | 1.00 | 24.01 | | A | N |
| ANISOU | 49 | N | ARG | A | 422 | 2992 | 3084 | 3046 | 16 | −1 | 27 | A | N |
| ATOM | 51 | CA | ARG | A | 422 | −11.041 | 22.756 | 25.366 | 1.00 | 22.30 | | A | C |
| ANISOU | 51 | CA | ARG | A | 422 | 2845 | 2819 | 2806 | 27 | 3 | 27 | A | C |
| ATOM | 53 | CB | ARG | A | 422 | −10.917 | 23.847 | 24.290 | 1.00 | 21.96 | | A | C |
| ANISOU | 53 | CB | ARG | A | 422 | 2771 | 2807 | 2766 | 36 | −4 | 32 | A | C |
| ATOM | 56 | CG | ARG | A | 422 | −9.490 | 24.349 | 24.085 | 1.00 | 21.12 | | A | C |
| ANISOU | 56 | CG | ARG | A | 422 | 2765 | 2618 | 2642 | −5 | 1 | 39 | A | C |
| ATOM | 59 | CD | ARG | A | 422 | −9.378 | 25.523 | 23.138 | 1.00 | 19.52 | | A | C |
| ANISOU | 59 | CD | ARG | A | 422 | 2505 | 2472 | 2436 | 15 | −28 | −27 | A | C |
| ATOM | 62 | NE | ARG | A | 422 | −9.899 | 25.202 | 21.812 | 1.00 | 18.34 | | A | N |
| ANISOU | 62 | NE | ARG | A | 422 | 2363 | 2273 | 2332 | 80 | −13 | 23 | A | N |
| ATOM | 64 | CZ | ARG | A | 422 | −9.213 | 24.608 | 20.840 | 1.00 | 16.29 | | A | C |
| ANISOU | 64 | CZ | ARG | A | 422 | 2110 | 2022 | 2056 | −41 | −63 | 7 | A | C |
| ATOM | 65 | NH1 | ARG | A | 422 | −7.965 | 24.214 | 21.025 | 1.00 | 14.80 | | A | N |
| ANISOU | 65 | NH1 | ARG | A | 422 | 2022 | 1925 | 1676 | −41 | −5 | −25 | A | N |
| ATOM | 68 | NH2 | ARG | A | 422 | −9.790 | 24.379 | 19.671 | 1.00 | 16.03 | | A | N |
| ANISOU | 68 | NH2 | ARG | A | 422 | 2044 | 2056 | 1991 | 55 | −106 | 122 | A | N |
| ATOM | 71 | C | ARG | A | 422 | −10.711 | 23.323 | 26.738 | 1.00 | 21.24 | | A | C |
| ANISOU | 71 | C | ARG | A | 422 | 2688 | 2703 | 2677 | 31 | 27 | 34 | A | C |
| ATOM | 72 | O | ARG | A | 422 | −9.578 | 23.209 | 27.188 | 1.00 | 20.08 | | A | O |
| ANISOU | 72 | O | ARG | A | 422 | 2563 | 2589 | 2475 | 42 | 80 | 71 | A | O |
| ATOM | 73 | N | GLU | A | 423 | −11.706 | 23.897 | 27.411 | 1.00 | 20.34 | | A | N |
| ANISOU | 73 | N | GLU | A | 423 | 2580 | 2572 | 2576 | 57 | −1 | 48 | A | N |
| ATOM | 75 | CA | GLU | A | 423 | −11.503 | 24.533 | 28.707 | 1.00 | 20.07 | | A | C |
| ANISOU | 75 | CA | GLU | A | 423 | 2550 | 2531 | 2542 | 52 | 1 | 38 | A | C |
| ATOM | 77 | CB | GLU | A | 423 | −12.724 | 25.406 | 29.090 | 1.00 | 20.68 | | A | C |
| ANISOU | 77 | CB | GLU | A | 423 | 2584 | 2640 | 2634 | 71 | 35 | 31 | A | C |
| ATOM | 80 | CG | GLU | A | 423 | −12.476 | 26.414 | 30.216 | 1.00 | 23.54 | | A | C |
| ANISOU | 80 | CG | GLU | A | 423 | 3027 | 3008 | 2907 | −79 | −35 | −10 | A | C |
| ATOM | 83 | CD | GLU | A | 423 | −13.574 | 27.477 | 30.362 | 1.00 | 26.82 | | A | C |
| ANISOU | 83 | CD | GLU | A | 423 | 3409 | 3383 | 3395 | 45 | −9 | 1 | A | C |
| ATOM | 84 | OE1 | GLU | A | 423 | −14.777 | 27.122 | 30.416 | 1.00 | 29.98 | | A | O |
| ANISOU | 84 | OE1 | GLU | A | 423 | 3624 | 3986 | 3778 | −102 | 18 | 77 | A | O |
| ATOM | 85 | OE2 | GLU | A | 423 | −13.251 | 28.688 | 30.433 | 1.00 | 27.61 | | A | O |
| ANISOU | 85 | OE2 | GLU | A | 423 | 3581 | 3449 | 3461 | −64 | −77 | −21 | A | O |
| ATOM | 86 | C | GLU | A | 423 | −11.209 | 23.499 | 29.810 | 1.00 | 18.81 | | A | C |
| ANISOU | 86 | C | GLU | A | 423 | 2381 | 2373 | 2390 | 43 | 19 | 20 | A | C |
| ATOM | 87 | O | GLU | A | 423 | −10.737 | 23.875 | 30.866 | 1.00 | 18.11 | | A | O |
| ANISOU | 87 | O | GLU | A | 423 | 2335 | 2198 | 2347 | 163 | 39 | 1 | A | O |
| ATOM | 88 | N | ASP | A | 424 | −11.483 | 22.214 | 29.555 | 1.00 | 17.37 | | A | N |
| ANISOU | 88 | N | ASP | A | 424 | 2170 | 2251 | 2179 | 93 | 40 | 40 | A | N |
| ATOM | 90 | CA | ASP | A | 424 | −11.106 | 21.134 | 30.486 | 1.00 | 16.78 | | A | C |
| ANISOU | 90 | CA | ASP | A | 424 | 2076 | 2178 | 2122 | 63 | 62 | 33 | A | C |
| ATOM | 92 | CB | ASP | A | 424 | −11.801 | 19.810 | 30.126 | 1.00 | 16.79 | | A | C |
| ANISOU | 92 | CB | ASP | A | 424 | 2124 | 2186 | 2066 | 65 | 15 | 51 | A | C |
| ATOM | 95 | CG | ASP | A | 424 | −13.310 | 19.872 | 30.235 | 1.00 | 18.67 | | A | C |
| ANISOU | 95 | CG | ASP | A | 424 | 2322 | 2411 | 2361 | −32 | 25 | 88 | A | C |
| ATOM | 96 | OD1 | ASP | A | 424 | −13.822 | 20.689 | 31.012 | 1.00 | 20.52 | | A | O |
| ANISOU | 96 | OD1 | ASP | A | 424 | 2501 | 2731 | 2565 | 98 | 55 | 2 | A | O |
| ATOM | 97 | OD2 | ASP | A | 424 | −14.059 | 19.117 | 29.590 | 1.00 | 20.81 | | A | O |
| ANISOU | 97 | OD2 | ASP | A | 424 | 2670 | 2802 | 2434 | −141 | −145 | 110 | A | O |
| ATOM | 98 | C | ASP | A | 424 | −9.591 | 20.887 | 30.510 | 1.00 | 16.59 | | A | C |
| ANISOU | 98 | C | ASP | A | 424 | 2045 | 2175 | 2084 | 58 | 19 | 79 | A | C |
| ATOM | 99 | O | ASP | A | 424 | −9.095 | 20.193 | 31.394 | 1.00 | 15.30 | | A | O |
| ANISOU | 99 | O | ASP | A | 424 | 1760 | 2109 | 1944 | 95 | 117 | 137 | A | O |
| ATOM | 100 | N | VAL | A | 425 | −8.866 | 21.439 | 29.537 | 1.00 | 15.79 | | A | N |
| ANISOU | 100 | N | VAL | A | 425 | 1968 | 2034 | 1998 | 81 | 36 | 53 | A | N |
| ATOM | 102 | CA | VAL | A | 425 | −7.433 | 21.180 | 29.400 | 1.00 | 16.53 | | A | C |
| ANISOU | 102 | CA | VAL | A | 425 | 2060 | 2116 | 2104 | 36 | 6 | 47 | A | C |
| ATOM | 104 | CB | VAL | A | 425 | −7.084 | 20.492 | 28.062 | 1.00 | 16.10 | | A | C |
| ANISOU | 104 | CB | VAL | A | 425 | 2052 | 2052 | 2014 | 63 | 6 | 94 | A | C |
| ATOM | 106 | CG1 | VAL | A | 425 | −5.577 | 20.299 | 27.943 | 1.00 | 17.51 | | A | C |
| ANISOU | 106 | CG1 | VAL | A | 425 | 2169 | 2241 | 2244 | 19 | 40 | 56 | A | C |
| ATOM | 110 | CG2 | VAL | A | 425 | −7.780 | 19.162 | 27.934 | 1.00 | 16.93 | | A | C |
| ANISOU | 110 | CG2 | VAL | A | 425 | 2140 | 2180 | 2111 | 13 | 45 | −29 | A | C |
| ATOM | 114 | C | VAL | A | 425 | −6.715 | 22.499 | 29.464 | 1.00 | 16.70 | | A | C |
| ANISOU | 114 | C | VAL | A | 425 | 2095 | 2109 | 2141 | 35 | 21 | 39 | A | C |
| ATOM | 115 | O | VAL | A | 425 | −7.006 | 23.392 | 28.650 | 1.00 | 17.77 | | A | O |
| ANISOU | 115 | O | VAL | A | 425 | 2229 | 2267 | 2253 | −18 | −21 | 164 | A | O |
| ATOM | 116 | N | VAL | A | 426 | −5.821 | 22.635 | 30.442 | 1.00 | 16.43 | | A | N |
| ANISOU | 116 | N | VAL | A | 426 | 2046 | 2074 | 2121 | 62 | −2 | 56 | A | N |
| ATOM | 118 | CA | VAL | A | 426 | −4.993 | 23.823 | 30.616 | 1.00 | 16.84 | | A | C |
| ANISOU | 118 | CA | VAL | A | 426 | 2106 | 2097 | 2194 | 49 | 16 | 37 | A | C |
| ATOM | 120 | CB | VAL | A | 426 | −5.078 | 24.352 | 32.052 | 1.00 | 17.23 | | A | C |
| ANISOU | 120 | CB | VAL | A | 426 | 2147 | 2187 | 2210 | 20 | −4 | 53 | A | C |
| ATOM | 122 | CG1 | VAL | A | 426 | −4.207 | 25.586 | 32.233 | 1.00 | 18.25 | | A | C |
| ANISOU | 122 | CG1 | VAL | A | 426 | 2299 | 2325 | 2309 | −30 | 3 | 0 | A | C |
| ATOM | 126 | CG2 | VAL | A | 426 | −6.534 | 24.674 | 32.402 | 1.00 | 17.11 | | A | C |
| ANISOU | 126 | CG2 | VAL | A | 426 | 2153 | 2093 | 2254 | 48 | 24 | 25 | A | C |
| ATOM | 130 | C | VAL | A | 426 | −3.534 | 23.506 | 30.292 | 1.00 | 16.82 | | A | C |
| ANISOU | 130 | C | VAL | A | 426 | 2114 | 2104 | 2170 | 38 | 43 | 46 | A | C |
| ATOM | 131 | O | VAL | A | 426 | −2.935 | 22.615 | 30.889 | 1.00 | 17.14 | | A | O |
| ANISOU | 131 | O | VAL | A | 426 | 2107 | 2165 | 2237 | 41 | 32 | 125 | A | O |
| ATOM | 132 | N | LEU | A | 427 | −2.973 | 24.235 | 29.340 | 1.00 | 16.42 | | A | N |
| ANISOU | 132 | N | LEU | A | 427 | 2112 | 1992 | 2133 | 31 | 37 | 13 | A | N |
| ATOM | 134 | CA | LEU | A | 427 | −1.595 | 24.028 | 28.926 | 1.00 | 16.16 | | A | C |
| ANISOU | 134 | CA | LEU | A | 427 | 2057 | 1994 | 2087 | −4 | 13 | −1 | A | C |
| ATOM | 136 | CB | LEU | A | 427 | −1.409 | 24.452 | 27.473 | 1.00 | 15.99 | | A | C |
| ANISOU | 136 | CB | LEU | A | 427 | 2026 | 2006 | 2044 | −10 | −4 | 0 | A | C |
| ATOM | 139 | CG | LEU | A | 427 | −2.397 | 23.859 | 26.453 | 1.00 | 15.54 | | A | C |
| ANISOU | 139 | CG | LEU | A | 427 | 1898 | 2022 | 1982 | −24 | 16 | 22 | A | C |
| ATOM | 141 | CD1 | LEU | A | 427 | −2.113 | 24.393 | 25.052 | 1.00 | 15.79 | | A | C |
| ANISOU | 141 | CD1 | LEU | A | 427 | 1866 | 2116 | 2017 | −36 | 35 | 23 | A | C |
| ATOM | 145 | CD2 | LEU | A | 427 | −2.417 | 22.333 | 26.481 | 1.00 | 15.07 | | A | C |
| ANISOU | 145 | CD2 | LEU | A | 427 | 1790 | 2024 | 1911 | −15 | 57 | 33 | A | C |
| ATOM | 149 | C | LEU | A | 427 | −0.667 | 24.823 | 29.826 | 1.00 | 16.42 | | A | C |
| ANISOU | 149 | C | LEU | A | 427 | 2122 | 2010 | 2105 | −15 | 11 | −28 | A | C |
| ATOM | 150 | O | LEU | A | 427 | −0.931 | 25.985 | 30.099 | 1.00 | 16.32 | | A | O |
| ANISOU | 150 | O | LEU | A | 427 | 2169 | 1842 | 2188 | 15 | 15 | 18 | A | O |
| ATOM | 151 | N | ASN | A | 428 | 0.417 | 24.199 | 30.284 | 1.00 | 16.65 | | A | N |
| ANISOU | 151 | N | ASN | A | 428 | 2139 | 2031 | 2154 | −4 | −2 | −29 | A | N |
| ATOM | 153 | CA | ASN | A | 428 | 1.375 | 24.848 | 31.192 | 1.00 | 17.77 | | A | C |
| ANISOU | 153 | CA | ASN | A | 428 | 2266 | 2207 | 2279 | −14 | −32 | −45 | A | C |
| ATOM | 155 | CB | ASN | A | 428 | 1.598 | 23.981 | 32.430 | 1.00 | 18.50 | | A | C |
| ANISOU | 155 | CB | ASN | A | 428 | 2377 | 2344 | 2306 | −40 | −65 | −41 | A | C |
| ATOM | 158 | CG | ASN | A | 428 | 0.304 | 23.646 | 33.156 | 1.00 | 20.68 | | A | C |
| ANISOU | 158 | CG | ASN | A | 428 | 2577 | 2704 | 2574 | −19 | −10 | −38 | A | C |
| ATOM | 159 | OD1 | ASN | A | 428 | 0.066 | 22.488 | 33.532 | 1.00 | 24.63 | | A | O |
| ANISOU | 159 | OD1 | ASN | A | 428 | 3195 | 3072 | 3091 | −115 | −67 | 140 | A | O |
| ATOM | 160 | ND2 | ASN | A | 428 | −0.544 | 24.648 | 33.345 | 1.00 | 23.16 | | A | N |
| ANISOU | 160 | ND2 | ASN | A | 428 | 2906 | 2948 | 2943 | 70 | 29 | −1 | A | N |
| ATOM | 163 | C | ASN | A | 428 | 2.731 | 25.180 | 30.562 | 1.00 | 18.24 | | A | C |
| ANISOU | 163 | C | ASN | A | 428 | 2302 | 2272 | 2355 | −18 | −21 | −40 | A | C |
| ATOM | 164 | O | ASN | A | 428 | 3.384 | 26.117 | 31.002 | 1.00 | 18.80 | | A | O |
| ANISOU | 164 | O | ASN | A | 428 | 2346 | 2345 | 2452 | −87 | −40 | −69 | A | O |
| ATOM | 165 | N | ARG | A | 429 | 3.178 | 24.391 | 29.582 | 1.00 | 18.33 | | A | N |
| ANISOU | 165 | N | ARG | A | 429 | 2318 | 2275 | 2371 | −42 | −38 | −47 | A | N |
| ATOM | 167 | CA | ARG | A | 429 | 4.441 | 24.649 | 28.874 | 1.00 | 18.98 | | A | C |
| ANISOU | 167 | CA | ARG | A | 429 | 2386 | 2394 | 2430 | −23 | −8 | −32 | A | C |
| ATOM | 169 | CB | ARG | A | 429 | 5.653 | 24.352 | 29.780 | 1.00 | 19.78 | | A | C |
| ANISOU | 169 | CB | ARG | A | 429 | 2501 | 2539 | 2476 | −3 | −45 | −60 | A | C |
| ATOM | 172 | CG | ARG | A | 429 | 5.760 | 22.912 | 30.242 | 1.00 | 22.03 | | A | C |
| ANISOU | 172 | CG | ARG | A | 429 | 2801 | 2757 | 2810 | −16 | 4 | 26 | A | C |
| ATOM | 175 | CD | ARG | A | 429 | 7.015 | 22.591 | 31.061 | 1.00 | 25.73 | | A | C |
| ANISOU | 175 | CD | ARG | A | 429 | 3174 | 3365 | 3234 | 32 | −104 | −3 | A | C |
| ATOM | 178 | NE | ARG | A | 429 | 8.241 | 23.038 | 30.394 | 1.00 | 27.84 | | A | N |
| ANISOU | 178 | NE | ARG | A | 429 | 3477 | 3553 | 3548 | −37 | 12 | 77 | A | N |
| ATOM | 180 | CZ | ARG | A | 429 | 9.067 | 22.276 | 29.671 | 1.00 | 29.91 | | A | C |
| ANISOU | 180 | CZ | ARG | A | 429 | 3750 | 3827 | 3785 | 28 | 2 | −36 | A | C |
| ATOM | 181 | NH1 | ARG | A | 429 | 8.851 | 20.976 | 29.496 | 1.00 | 31.32 | | A | N |
| ANISOU | 181 | NH1 | ARG | A | 429 | 3973 | 3878 | 4047 | −4 | 11 | −1 | A | N |
| ATOM | 184 | NH2 | ARG | A | 429 | 10.143 | 22.825 | 29.125 | 1.00 | 31.16 | | A | N |
| ANISOU | 184 | NH2 | ARG | A | 429 | 3955 | 3964 | 3918 | −21 | 59 | 65 | A | N |
| ATOM | 187 | C | ARG | A | 429 | 4.572 | 23.855 | 27.578 | 1.00 | 18.59 | | A | C |
| ANISOU | 187 | C | ARG | A | 429 | 2338 | 2319 | 2403 | −35 | −13 | −39 | A | C |
| ATOM | 188 | O | ARG | A | 429 | 3.769 | 22.957 | 27.324 | 1.00 | 17.92 | | A | O |
| ANISOU | 188 | O | ARG | A | 429 | 2141 | 2343 | 2323 | −126 | −43 | −100 | A | O |
| ATOM | 189 | N | ILE | A | 430 | 5.576 | 24.176 | 26.762 | 1.00 | 18.50 | | A | N |
| ANISOU | 189 | N | ILE | A | 430 | 2310 | 2296 | 2421 | −75 | −32 | −40 | A | N |
| ATOM | 191 | CA | ILE | A | 430 | 5.883 | 23.381 | 25.572 | 1.00 | 19.36 | | A | C |
| ANISOU | 191 | CA | ILE | A | 430 | 2429 | 2451 | 2475 | −26 | 8 | −3 | A | C |
| ATOM | 193 | CB | ILE | A | 430 | 6.305 | 24.277 | 24.350 | 1.00 | 19.16 | | A | C |
| ANISOU | 193 | CB | ILE | A | 430 | 2389 | 2413 | 2478 | −29 | 2 | 2 | A | C |
| ATOM | 195 | CG1 | ILE | A | 430 | 5.082 | 24.971 | 23.769 | 1.00 | 18.82 | | A | C |
| ANISOU | 195 | CG1 | ILE | A | 430 | 2379 | 2336 | 2433 | −52 | 39 | 34 | A | C |
| ATOM | 198 | CD1 | ILE | A | 430 | 5.354 | 26.108 | 22.766 | 1.00 | 17.44 | | A | C |
| ANISOU | 198 | CD1 | ILE | A | 430 | 2197 | 2173 | 2255 | −15 | 2 | −12 | A | C |
| ATOM | 202 | CG2 | ILE | A | 430 | 6.954 | 23.428 | 23.250 | 1.00 | 20.27 | | A | C |
| ANISOU | 202 | CG2 | ILE | A | 430 | 2545 | 2602 | 2553 | −30 | 52 | −11 | A | C |
| ATOM | 206 | C | ILE | A | 430 | 6.958 | 22.359 | 25.913 | 1.00 | 20.29 | | A | C |
| ANISOU | 206 | C | ILE | A | 430 | 2520 | 2613 | 2574 | −3 | −29 | 9 | A | C |
| ATOM | 207 | O | ILE | A | 430 | 8.054 | 22.722 | 26.357 | 1.00 | 20.11 | | A | O |
| ANISOU | 207 | O | ILE | A | 430 | 2489 | 2615 | 2536 | −28 | −52 | −14 | A | O |
| ATOM | 208 | N | LEU | A | 431 | 6.624 | 21.084 | 25.721 | 1.00 | 21.10 | | A | N |
| ANISOU | 208 | N | LEU | A | 431 | 2647 | 2706 | 2664 | −14 | −24 | −36 | A | N |
| ATOM | 210 | CA | LEU | A | 431 | 7.550 | 19.968 | 25.917 | 1.00 | 22.54 | | A | C |
| ANISOU | 210 | CA | LEU | A | 431 | 2810 | 2899 | 2856 | 18 | −27 | 8 | A | C |
| ATOM | 212 | CB | LEU | A | 431 | 6.799 | 18.635 | 25.897 | 1.00 | 22.99 | | A | C |
| ANISOU | 212 | CB | LEU | A | 431 | 2892 | 2919 | 2922 | 13 | −16 | −11 | A | C |
| ATOM | 215 | CG | LEU | A | 431 | 5.887 | 18.344 | 27.086 | 1.00 | 23.65 | | A | C |
| ANISOU | 215 | CG | LEU | A | 431 | 2963 | 3032 | 2990 | 7 | −2 | 16 | A | C |
| ATOM | 217 | CD1 | LEU | A | 431 | 5.020 | 17.147 | 26.774 | 1.00 | 24.23 | | A | C |
| ANISOU | 217 | CD1 | LEU | A | 431 | 3139 | 2966 | 3100 | 34 | 1 | 19 | A | C |
| ATOM | 221 | CD2 | LEU | A | 431 | 6.687 | 18.129 | 28.377 | 1.00 | 25.23 | | A | C |
| ANISOU | 221 | CD2 | LEU | A | 431 | 3176 | 3233 | 3175 | 7 | −73 | 17 | A | C |
| ATOM | 225 | C | LEU | A | 431 | 8.604 | 19.930 | 24.829 | 1.00 | 23.65 | | A | C |
| ANISOU | 225 | C | LEU | A | 431 | 2949 | 3070 | 2967 | 30 | −5 | 5 | A | C |
| ATOM | 226 | O | LEU | A | 431 | 9.779 | 19.671 | 25.090 | 1.00 | 24.47 | | A | O |
| ANISOU | 226 | O | LEU | A | 431 | 2919 | 3312 | 3064 | 91 | 15 | 44 | A | O |
| ATOM | 227 | N | GLY | A | 432 | 8.181 | 20.182 | 23.601 | 1.00 | 24.46 | | A | N |
| ANISOU | 227 | N | GLY | A | 432 | 3052 | 3167 | 3073 | 23 | −23 | 9 | A | N |
| ATOM | 229 | CA | GLY | A | 432 | 9.096 | 20.197 | 22.483 | 1.00 | 24.89 | | A | C |
| ANISOU | 229 | CA | GLY | A | 432 | 3127 | 3179 | 3149 | 33 | 14 | 9 | A | C |
| ATOM | 232 | C | GLY | A | 432 | 8.370 | 20.267 | 21.164 | 1.00 | 25.37 | | A | C |
| ANISOU | 232 | C | GLY | A | 432 | 3191 | 3230 | 3215 | 1 | −9 | −6 | A | C |
| ATOM | 233 | O | GLY | A | 432 | 7.138 | 20.300 | 21.119 | 1.00 | 25.05 | | A | O |
| ANISOU | 233 | O | GLY | A | 432 | 3135 | 3225 | 3156 | 0 | 0 | −2 | A | O |
| ATOM | 234 | N | GLU | A | 433 | 9.147 | 20.306 | 20.092 | 1.00 | 26.02 | | A | N |
| ANISOU | 234 | N | GLU | A | 433 | 3270 | 3304 | 3310 | 14 | 34 | −7 | A | N |
| ATOM | 236 | CA | GLU | A | 433 | 8.614 | 20.321 | 18.743 | 1.00 | 26.75 | | A | C |
| ANISOU | 236 | CA | GLU | A | 433 | 3355 | 3403 | 3403 | 14 | 7 | −12 | A | C |
| ATOM | 238 | CB | GLU | A | 433 | 9.483 | 21.193 | 17.829 | 1.00 | 26.94 | | A | C |
| ANISOU | 238 | CB | GLU | A | 433 | 3392 | 3422 | 3420 | 2 | 19 | 18 | A | C |
| ATOM | 241 | CG | GLU | A | 433 | 9.341 | 22.703 | 18.053 | 1.00 | 27.97 | | A | C |
| ANISOU | 241 | CG | GLU | A | 433 | 3539 | 3515 | 3571 | 14 | 29 | −17 | A | C |
| ATOM | 244 | CD | GLU | A | 433 | 10.146 | 23.249 | 19.235 | 1.00 | 29.18 | | A | C |
| ANISOU | 244 | CD | GLU | A | 433 | 3665 | 3749 | 3670 | −3 | −18 | −15 | A | C |
| ATOM | 245 | OE1 | GLU | A | 433 | 11.395 | 23.216 | 19.160 | 1.00 | 30.69 | | A | O |
| ANISOU | 245 | OE1 | GLU | A | 433 | 3750 | 4008 | 3900 | 2 | −10 | −14 | A | O |
| ATOM | 246 | OE2 | GLU | A | 433 | 9.541 | 23.737 | 20.234 | 1.00 | 28.38 | | A | O |
| ANISOU | 246 | OE2 | GLU | A | 433 | 3532 | 3554 | 3696 | 25 | −23 | 16 | A | O |
| ATOM | 247 | C | GLU | A | 433 | 8.571 | 18.881 | 18.235 | 1.00 | 27.15 | | A | C |
| ANISOU | 247 | C | GLU | A | 433 | 3398 | 3442 | 3475 | 10 | 27 | −21 | A | C |
| ATOM | 248 | O | GLU | A | 433 | 9.585 | 18.323 | 17.795 | 1.00 | 28.41 | | A | O |
| ANISOU | 248 | O | GLU | A | 433 | 3499 | 3633 | 3663 | 90 | 49 | −61 | A | O |
| ATOM | 249 | N | GLY | A | 434 | 7.405 | 18.262 | 18.331 | 1.00 | 26.92 | | A | N |
| ANISOU | 249 | N | GLY | A | 434 | 3365 | 3436 | 3426 | 13 | 11 | −13 | A | N |
| ATOM | 251 | CA | GLY | A | 434 | 7.194 | 16.951 | 17.757 | 1.00 | 26.85 | | A | C |
| ANISOU | 251 | CA | GLY | A | 434 | 3389 | 3412 | 3398 | 9 | 6 | −6 | A | C |
| ATOM | 254 | C | GLY | A | 434 | 7.116 | 16.989 | 16.237 | 1.00 | 26.79 | | A | C |
| ANISOU | 254 | C | GLY | A | 434 | 3399 | 3396 | 3383 | 0 | 8 | −15 | A | C |
| ATOM | 255 | O | GLY | A | 434 | 7.243 | 18.048 | 15.600 | 1.00 | 26.22 | | A | O |
| ANISOU | 255 | O | GLY | A | 434 | 3310 | 3352 | 3297 | −17 | 52 | −68 | A | O |
| ATOM | 256 | N | PHE | A | 435 | 6.896 | 15.813 | 15.658 | 1.00 | 27.04 | | A | N |
| ANISOU | 256 | N | PHE | A | 435 | 3441 | 3400 | 3431 | 8 | 1 | −23 | A | N |
| ATOM | 258 | CA | PHE | A | 435 | 6.782 | 15.647 | 14.207 | 1.00 | 27.52 | | A | C |
| ANISOU | 258 | CA | PHE | A | 435 | 3512 | 3474 | 3469 | 23 | 7 | −7 | A | C |
| ATOM | 260 | CB | PHE | A | 435 | 6.369 | 14.201 | 13.893 | 1.00 | 28.44 | | A | C |
| ANISOU | 260 | CB | PHE | A | 435 | 3608 | 3563 | 3632 | 18 | 0 | −16 | A | C |
| ATOM | 263 | CG | PHE | A | 435 | 6.300 | 13.899 | 12.426 | 1.00 | 31.95 | | A | C |
| ANISOU | 263 | CG | PHE | A | 435 | 4122 | 4087 | 3929 | 16 | −14 | −47 | A | C |
| ATOM | 264 | CD1 | PHE | A | 435 | 7.460 | 13.682 | 11.697 | 1.00 | 34.08 | | A | C |
| ANISOU | 264 | CD1 | PHE | A | 435 | 4267 | 4393 | 4289 | 29 | 77 | 0 | A | C |
| ATOM | 266 | CE1 | PHE | A | 435 | 7.402 | 13.403 | 10.343 | 1.00 | 35.40 | | A | C |
| ANISOU | 266 | CE1 | PHE | A | 435 | 4500 | 4563 | 4384 | 4 | −24 | −22 | A | C |
| ATOM | 268 | CZ | PHE | A | 435 | 6.182 | 13.346 | 9.707 | 1.00 | 35.84 | | A | C |
| ANISOU | 268 | CZ | PHE | A | 435 | 4486 | 4620 | 4512 | 12 | 3 | −13 | A | C |
| ATOM | 270 | CE2 | PHE | A | 435 | 5.017 | 13.571 | 10.423 | 1.00 | 35.45 | | A | C |
| ANISOU | 270 | CE2 | PHE | A | 435 | 4516 | 4558 | 4392 | 2 | −1 | −25 | A | C |
| ATOM | 272 | CD2 | PHE | A | 435 | 5.081 | 13.845 | 11.773 | 1.00 | 34.18 | | A | C |
| ANISOU | 272 | CD2 | PHE | A | 435 | 4255 | 4412 | 4317 | 14 | −54 | −23 | A | C |
| ATOM | 274 | C | PHE | A | 435 | 5.759 | 16.589 | 13.575 | 1.00 | 26.36 | | A | C |
| ANISOU | 274 | C | PHE | A | 435 | 3364 | 3309 | 3342 | −1 | 26 | −19 | A | C |
| ATOM | 275 | O | PHE | A | 435 | 6.031 | 17.213 | 12.544 | 1.00 | 26.24 | | A | O |
| ANISOU | 275 | O | PHE | A | 435 | 3407 | 3284 | 3278 | 25 | 77 | −80 | A | O |
| ATOM | 276 | N | PHE | A | 436 | 4.587 | 16.674 | 14.208 | 1.00 | 25.12 | | A | N |
| ANISOU | 276 | N | PHE | A | 436 | 3244 | 3148 | 3152 | 36 | 13 | −19 | A | N |
| ATOM | 278 | CA | PHE | A | 436 | 3.429 | 17.402 | 13.675 | 1.00 | 24.06 | | A | C |
| ANISOU | 278 | CA | PHE | A | 436 | 3094 | 3015 | 3031 | 8 | 17 | −49 | A | C |
| ATOM | 280 | CB | PHE | A | 436 | 2.124 | 16.826 | 14.254 | 1.00 | 24.95 | | A | C |
| ANISOU | 280 | CB | PHE | A | 436 | 3157 | 3180 | 3142 | 19 | 12 | −9 | A | C |
| ATOM | 283 | CG | PHE | A | 436 | 1.940 | 15.351 | 14.009 | 1.00 | 28.95 | | A | C |
| ANISOU | 283 | CG | PHE | A | 436 | 3718 | 3538 | 3741 | −72 | 13 | −29 | A | C |
| ATOM | 284 | CD1 | PHE | A | 436 | 1.704 | 14.872 | 12.729 | 1.00 | 31.70 | | A | C |
| ANISOU | 284 | CD1 | PHE | A | 436 | 4101 | 4031 | 3910 | −16 | −55 | −61 | A | C |
| ATOM | 286 | CE1 | PHE | A | 436 | 1.541 | 13.498 | 12.497 | 1.00 | 33.36 | | A | C |
| ANISOU | 286 | CE1 | PHE | A | 436 | 4325 | 4099 | 4251 | −26 | −5 | 5 | A | C |
| ATOM | 288 | CZ | PHE | A | 436 | 1.612 | 12.604 | 13.552 | 1.00 | 33.62 | | A | C |
| ANISOU | 288 | CZ | PHE | A | 436 | 4343 | 4232 | 4199 | 14 | −31 | −2 | A | C |
| ATOM | 290 | CE2 | PHE | A | 436 | 1.841 | 13.069 | 14.839 | 1.00 | 33.02 | | A | C |
| ANISOU | 290 | CE2 | PHE | A | 436 | 4264 | 4081 | 4198 | −24 | −20 | −42 | A | C |
| ATOM | 292 | CD2 | PHE | A | 436 | 2.014 | 14.436 | 15.062 | 1.00 | 31.64 | | A | C |
| ANISOU | 292 | CD2 | PHE | A | 436 | 4094 | 3946 | 3979 | 3 | −42 | 86 | A | C |
| ATOM | 294 | C | PHE | A | 436 | 3.464 | 18.893 | 14.013 | 1.00 | 21.43 | | A | C |
| ANISOU | 294 | C | PHE | A | 436 | 2729 | 2772 | 2639 | −19 | 23 | −10 | A | C |
| ATOM | 295 | O | PHE | A | 436 | 2.847 | 19.695 | 13.329 | 1.00 | 19.47 | | A | O |
| ANISOU | 295 | O | PHE | A | 436 | 2560 | 2502 | 2335 | −26 | 134 | −54 | A | O |
| ATOM | 296 | N | GLY | A | 437 | 4.152 | 19.237 | 15.099 | 1.00 | 18.75 | | A | N |
| ANISOU | 296 | N | GLY | A | 437 | 2360 | 2386 | 2379 | 8 | 78 | −34 | A | N |
| ATOM | 298 | CA | GLY | A | 437 | 4.094 | 20.576 | 15.662 | 1.00 | 17.07 | | A | C |
| ANISOU | 298 | CA | GLY | A | 437 | 2190 | 2193 | 2101 | −57 | 32 | −19 | A | C |
| ATOM | 301 | C | GLY | A | 437 | 4.424 | 20.600 | 17.144 | 1.00 | 15.71 | | A | C |
| ANISOU | 301 | C | GLY | A | 437 | 2041 | 1958 | 1969 | −1 | 43 | −18 | A | C |
| ATOM | 302 | O | GLY | A | 437 | 4.907 | 19.609 | 17.708 | 1.00 | 14.91 | | A | O |
| ANISOU | 302 | O | GLY | A | 437 | 1941 | 1977 | 1747 | −16 | 42 | −135 | A | O |
| ATOM | 303 | N | GLU | A | 438 | 4.123 | 21.731 | 17.791 | 1.00 | 14.54 | | A | N |
| ANISOU | 303 | N | GLU | A | 438 | 1846 | 1917 | 1760 | −54 | 62 | −10 | A | N |
| ATOM | 305 | CA | GLU | A | 438 | 4.468 | 21.899 | 19.191 | 1.00 | 14.13 | | A | C |
| ANISOU | 305 | CA | GLU | A | 438 | 1791 | 1840 | 1738 | −6 | 38 | −4 | A | C |
| ATOM | 307 | CB | GLU | A | 438 | 4.184 | 23.318 | 19.661 | 1.00 | 14.11 | | A | C |
| ANISOU | 307 | CB | GLU | A | 438 | 1813 | 1794 | 1754 | −23 | 47 | 6 | A | C |
| ATOM | 310 | CG | GLU | A | 438 | 4.960 | 24.388 | 18.916 | 1.00 | 16.03 | | A | C |
| ANISOU | 310 | CG | GLU | A | 438 | 2081 | 2028 | 1979 | −54 | 110 | 73 | A | C |
| ATOM | 313 | CD | GLU | A | 438 | 6.368 | 24.563 | 19.416 | 1.00 | 18.92 | | A | C |
| ANISOU | 313 | CD | GLU | A | 438 | 2336 | 2458 | 2391 | −20 | −14 | 61 | A | C |
| ATOM | 314 | OE1 | GLU | A | 438 | 6.963 | 23.598 | 19.938 | 1.00 | 18.75 | | A | O |
| ANISOU | 314 | OE1 | GLU | A | 438 | 2304 | 2342 | 2479 | 5 | 183 | 65 | A | O |
| ATOM | 315 | OE2 | GLU | A | 438 | 6.884 | 25.699 | 19.299 | 1.00 | 22.36 | | A | O |
| ANISOU | 315 | OE2 | GLU | A | 438 | 2910 | 2643 | 2941 | −115 | 55 | 23 | A | O |
| ATOM | 316 | C | GLU | A | 438 | 3.635 | 20.925 | 20.012 | 1.00 | 13.22 | | A | C |
| ANISOU | 316 | C | GLU | A | 438 | 1653 | 1763 | 1605 | 20 | 46 | −9 | A | C |
| ATOM | 317 | O | GLU | A | 438 | 2.459 | 20.714 | 19.735 | 1.00 | 13.07 | | A | O |
| ANISOU | 317 | O | GLU | A | 438 | 1730 | 1939 | 1297 | −74 | 72 | −60 | A | O |
| ATOM | 318 | N | VAL | A | 439 | 4.264 | 20.330 | 21.014 | 1.00 | 12.73 | | A | N |
| ANISOU | 318 | N | VAL | A | 439 | 1567 | 1724 | 1544 | −23 | 67 | 16 | A | N |
| ATOM | 320 | CA | VAL | A | 439 | 3.598 | 19.457 | 21.963 | 1.00 | 11.95 | | A | C |
| ANISOU | 320 | CA | VAL | A | 439 | 1504 | 1539 | 1497 | −17 | 45 | 2 | A | C |
| ATOM | 322 | CB | VAL | A | 439 | 4.260 | 18.070 | 22.058 | 1.00 | 12.24 | | A | C |
| ANISOU | 322 | CB | VAL | A | 439 | 1482 | 1624 | 1542 | −19 | 10 | −21 | A | C |
| ATOM | 324 | CG1 | VAL | A | 439 | 3.502 | 17.197 | 23.059 | 1.00 | 12.64 | | A | C |
| ANISOU | 324 | CG1 | VAL | A | 439 | 1592 | 1602 | 1608 | 62 | −44 | 85 | A | C |
| ATOM | 328 | CG2 | VAL | A | 439 | 4.322 | 17.420 | 20.693 | 1.00 | 12.04 | | A | C |
| ANISOU | 328 | CG2 | VAL | A | 439 | 1607 | 1347 | 1619 | −24 | 106 | −32 | A | C |
| ATOM | 332 | C | VAL | A | 439 | 3.684 | 20.154 | 23.315 | 1.00 | 11.31 | | A | C |
| ANISOU | 332 | C | VAL | A | 439 | 1386 | 1473 | 1435 | −72 | −15 | −9 | A | C |
| ATOM | 333 | O | VAL | A | 439 | 4.759 | 20.594 | 23.714 | 1.00 | 11.12 | | A | O |
| ANISOU | 333 | O | VAL | A | 439 | 1313 | 1556 | 1355 | −141 | 6 | −48 | A | O |
| ATOM | 334 | N | TYR | A | 440 | 2.549 | 20.243 | 24.010 | 1.00 | 10.48 | | A | N |
| ANISOU | 334 | N | TYR | A | 440 | 1319 | 1294 | 1369 | −93 | 7 | −21 | A | N |
| ATOM | 336 | CA | TYR | A | 440 | 2.442 | 20.922 | 25.290 | 1.00 | 10.54 | | A | C |
| ANISOU | 336 | CA | TYR | A | 440 | 1302 | 1358 | 1344 | −83 | −23 | 11 | A | C |
| ATOM | 338 | CB | TYR | A | 440 | 1.274 | 21.906 | 25.265 | 1.00 | 10.13 | | A | C |
| ANISOU | 338 | CB | TYR | A | 440 | 1292 | 1296 | 1258 | −67 | −30 | −4 | A | C |
| ATOM | 341 | CG | TYR | A | 440 | 1.357 | 22.990 | 24.208 | 1.00 | 9.13 | | A | C |
| ANISOU | 341 | CG | TYR | A | 440 | 1147 | 1189 | 1134 | −97 | 51 | −19 | A | C |
| ATOM | 342 | CD1 | TYR | A | 440 | 1.833 | 24.248 | 24.511 | 1.00 | 10.81 | | A | C |
| ANISOU | 342 | CD1 | TYR | A | 440 | 1463 | 1310 | 1334 | −114 | −80 | 2 | A | C |
| ATOM | 344 | CE1 | TYR | A | 440 | 1.897 | 25.241 | 23.543 | 1.00 | 12.32 | | A | C |
| ANISOU | 344 | CE1 | TYR | A | 440 | 1734 | 1562 | 1382 | −166 | −18 | 76 | A | C |
| ATOM | 346 | CZ | TYR | A | 440 | 1.515 | 24.972 | 22.249 | 1.00 | 11.63 | | A | C |
| ANISOU | 346 | CZ | TYR | A | 440 | 1604 | 1416 | 1396 | −86 | 0 | 91 | A | C |
| ATOM | 347 | OH | TYR | A | 440 | 1.618 | 25.996 | 21.316 | 1.00 | 13.82 | | A | O |
| ANISOU | 347 | OH | TYR | A | 440 | 1852 | 1892 | 1504 | −314 | 45 | 253 | A | O |
| ATOM | 349 | CE2 | TYR | A | 440 | 1.065 | 23.727 | 21.917 | 1.00 | 10.58 | | A | C |
| ANISOU | 349 | CE2 | TYR | A | 440 | 1356 | 1517 | 1147 | −56 | 79 | −18 | A | C |
| ATOM | 351 | CD2 | TYR | A | 440 | 0.996 | 22.737 | 22.891 | 1.00 | 9.78 | | A | C |
| ANISOU | 351 | CD2 | TYR | A | 440 | 1196 | 1328 | 1190 | −90 | −59 | −9 | A | C |
| ATOM | 353 | C | TYR | A | 440 | 2.196 | 19.940 | 26.414 | 1.00 | 11.30 | | A | C |
| ANISOU | 353 | C | TYR | A | 440 | 1409 | 1422 | 1462 | −125 | −34 | 5 | A | C |
| ATOM | 354 | O | TYR | A | 440 | 1.523 | 18.932 | 26.226 | 1.00 | 11.93 | | A | O |
| ANISOU | 354 | O | TYR | A | 440 | 1520 | 1449 | 1560 | −275 | −170 | −31 | A | O |
| ATOM | 355 | N | GLU | A | 441 | 2.724 | 20.239 | 27.593 | 1.00 | 11.57 | | A | N |
| ANISOU | 355 | N | GLU | A | 441 | 1424 | 1507 | 1464 | −80 | −52 | −40 | A | N |
| ATOM | 357 | CA | GLU | A | 441 | 2.312 | 19.572 | 28.810 | 1.00 | 12.96 | | A | C |
| ANISOU | 357 | CA | GLU | A | 441 | 1633 | 1646 | 1645 | −13 | −4 | −27 | A | C |
| ATOM | 359 | CB | GLU | A | 441 | 3.418 | 19.564 | 29.862 | 1.00 | 14.31 | | A | C |
| ANISOU | 359 | CB | GLU | A | 441 | 1748 | 1885 | 1804 | 24 | −44 | −27 | A | C |
| ATOM | 362 | CG | GLU | A | 441 | 3.021 | 18.766 | 31.094 | 1.00 | 18.27 | | A | C |
| ANISOU | 362 | CG | GLU | A | 441 | 2323 | 2331 | 2285 | −75 | 92 | 40 | A | C |
| ATOM | 365 | CD | GLU | A | 441 | 4.123 | 18.618 | 32.115 | 1.00 | 23.03 | | A | C |
| ANISOU | 365 | CD | GLU | A | 441 | 2875 | 3010 | 2863 | 41 | −140 | 55 | A | C |
| ATOM | 366 | OE1 | GLU | A | 441 | 5.321 | 18.774 | 31.760 | 1.00 | 27.63 | | A | O |
| ANISOU | 366 | OE1 | GLU | A | 441 | 3187 | 3829 | 3479 | 15 | 35 | 124 | A | O |
| ATOM | 367 | OE2 | GLU | A | 441 | 3.786 | 18.342 | 33.285 | 1.00 | 26.26 | | A | O |
| ANISOU | 367 | OE2 | GLU | A | 441 | 3301 | 3543 | 3134 | 26 | 99 | 36 | A | O |
| ATOM | 368 | C | GLU | A | 441 | 1.140 | 20.351 | 29.342 | 1.00 | 12.30 | | A | C |
| ANISOU | 368 | C | GLU | A | 441 | 1566 | 1580 | 1527 | 5 | −14 | −32 | A | C |
| ATOM | 369 | O | GLU | A | 441 | 1.151 | 21.596 | 29.341 | 1.00 | 12.57 | | A | O |
| ANISOU | 369 | O | GLU | A | 441 | 1640 | 1579 | 1554 | −29 | 34 | −28 | A | O |
| ATOM | 370 | N | GLY | A | 442 | 0.118 | 19.634 | 29.769 | 1.00 | 12.18 | | A | N |
| ANISOU | 370 | N | GLY | A | 442 | 1611 | 1531 | 1486 | 14 | 21 | −26 | A | N |
| ATOM | 372 | CA | GLY | A | 442 | −1.001 | 20.257 | 30.452 | 1.00 | 12.50 | | A | C |
| ANISOU | 372 | CA | GLY | A | 442 | 1603 | 1609 | 1534 | 15 | 12 | −27 | A | C |
| ATOM | 375 | C | GLY | A | 442 | −1.686 | 19.352 | 31.454 | 1.00 | 12.64 | | A | C |
| ANISOU | 375 | C | GLY | A | 442 | 1609 | 1602 | 1592 | 0 | −13 | 2 | A | C |
| ATOM | 376 | O | GLY | A | 442 | −1.237 | 18.246 | 31.752 | 1.00 | 12.27 | | A | O |
| ANISOU | 376 | O | GLY | A | 442 | 1662 | 1541 | 1457 | −49 | −21 | −114 | A | O |
| ATOM | 377 | N | VAL | A | 443 | −2.807 | 19.848 | 31.963 | 1.00 | 13.10 | | A | N |
| ANISOU | 377 | N | VAL | A | 443 | 1693 | 1666 | 1616 | 27 | 35 | 7 | A | N |
| ATOM | 379 | CA | VAL | A | 443 | −3.639 | 19.103 | 32.889 | 1.00 | 13.71 | | A | C |
| ANISOU | 379 | CA | VAL | A | 443 | 1738 | 1730 | 1741 | 13 | 14 | 18 | A | C |
| ATOM | 381 | CB | VAL | A | 443 | −3.578 | 19.703 | 34.298 | 1.00 | 14.27 | | A | C |
| ANISOU | 381 | CB | VAL | A | 443 | 1788 | 1845 | 1786 | −11 | 3 | −5 | A | C |
| ATOM | 383 | CG1 | VAL | A | 443 | −4.581 | 19.007 | 35.233 | 1.00 | 14.54 | | A | C |
| ANISOU | 383 | CG1 | VAL | A | 443 | 1859 | 1914 | 1752 | 22 | 34 | 38 | A | C |
| ATOM | 387 | CG2 | VAL | A | 443 | −2.181 | 19.561 | 34.859 | 1.00 | 14.32 | | A | C |
| ANISOU | 387 | CG2 | VAL | A | 443 | 1795 | 1872 | 1773 | 5 | −10 | −59 | A | C |
| ATOM | 391 | C | VAL | A | 443 | −5.074 | 19.067 | 32.403 | 1.00 | 13.17 | | A | C |
| ANISOU | 391 | C | VAL | A | 443 | 1686 | 1660 | 1657 | 20 | 35 | 41 | A | C |
| ATOM | 392 | O | VAL | A | 443 | −5.677 | 20.094 | 32.118 | 1.00 | 13.26 | | A | O |
| ANISOU | 392 | O | VAL | A | 443 | 1742 | 1581 | 1716 | 67 | 43 | 55 | A | O |
| ATOM | 393 | N | TYR | A | 444 | −5.619 | 17.864 | 32.342 | 1.00 | 12.72 | | A | N |
| ANISOU | 393 | N | TYR | A | 444 | 1678 | 1575 | 1578 | 54 | 22 | 66 | A | N |
| ATOM | 395 | CA | TYR | A | 444 | −6.976 | 17.622 | 31.944 | 1.00 | 12.83 | | A | C |
| ANISOU | 395 | CA | TYR | A | 444 | 1637 | 1633 | 1604 | 43 | 50 | 91 | A | C |
| ATOM | 397 | CB | TYR | A | 444 | −7.041 | 16.415 | 31.015 | 1.00 | 13.36 | | A | C |
| ANISOU | 397 | CB | TYR | A | 444 | 1722 | 1673 | 1680 | 21 | 40 | 108 | A | C |
| ATOM | 400 | CG | TYR | A | 444 | −8.425 | 15.829 | 30.850 | 1.00 | 14.22 | | A | C |
| ANISOU | 400 | CG | TYR | A | 444 | 1777 | 1870 | 1756 | −4 | 32 | 88 | A | C |
| ATOM | 401 | CD1 | TYR | A | 444 | −9.527 | 16.644 | 30.596 | 1.00 | 15.71 | | A | C |
| ANISOU | 401 | CD1 | TYR | A | 444 | 1985 | 2023 | 1959 | 40 | 13 | 81 | A | C |
| ATOM | 403 | CE1 | TYR | A | 444 | −10.793 | 16.095 | 30.460 | 1.00 | 16.75 | | A | C |
| ANISOU | 403 | CE1 | TYR | A | 444 | 2039 | 2253 | 2071 | 7 | −72 | 99 | A | C |
| ATOM | 405 | CZ | TYR | A | 444 | −10.954 | 14.728 | 30.556 | 1.00 | 17.08 | | A | C |
| ANISOU | 405 | CZ | TYR | A | 444 | 2181 | 2265 | 2042 | −6 | 6 | 32 | A | C |
| ATOM | 406 | OH | TYR | A | 444 | −12.193 | 14.156 | 30.422 | 1.00 | 20.54 | | A | O |
| ANISOU | 406 | OH | TYR | A | 444 | 2562 | 2665 | 2575 | −236 | −38 | 65 | A | O |
| ATOM | 408 | CE2 | TYR | A | 444 | −9.890 | 13.917 | 30.810 | 1.00 | 16.31 | | A | C |
| ANISOU | 408 | CE2 | TYR | A | 444 | 2117 | 2000 | 2077 | −72 | 6 | 64 | A | C |
| ATOM | 410 | CD2 | TYR | A | 444 | −8.631 | 14.462 | 30.943 | 1.00 | 16.63 | | A | C |
| ANISOU | 410 | CD2 | TYR | A | 444 | 2057 | 2040 | 2221 | −12 | 36 | 28 | A | C |
| ATOM | 412 | C | TYR | A | 444 | −7.735 | 17.350 | 33.218 | 1.00 | 13.67 | | A | C |
| ANISOU | 412 | C | TYR | A | 444 | 1762 | 1727 | 1703 | 52 | 59 | 79 | A | C |
| ATOM | 413 | O | TYR | A | 444 | −7.379 | 16.436 | 33.953 | 1.00 | 13.60 | | A | O |
| ANISOU | 413 | O | TYR | A | 444 | 1743 | 1767 | 1656 | 42 | 51 | 153 | A | O |
| ATOM | 414 | N | THR | A | 445 | −8.749 | 18.172 | 33.485 | 1.00 | 13.86 | | A | N |
| ANISOU | 414 | N | THR | A | 445 | 1723 | 1781 | 1762 | 60 | 72 | 36 | A | N |
| ATOM | 416 | CA | THR | A | 445 | −9.588 | 18.011 | 34.665 | 1.00 | 14.76 | | A | C |
| ANISOU | 416 | CA | THR | A | 445 | 1875 | 1910 | 1821 | 0 | 43 | 60 | A | C |
| ATOM | 418 | CB | THR | A | 445 | −9.814 | 19.361 | 35.380 | 1.00 | 14.26 | | A | C |
| ANISOU | 418 | CB | THR | A | 445 | 1780 | 1837 | 1800 | 41 | 58 | 72 | A | C |
| ATOM | 420 | OG1 | THR | A | 445 | −8.561 | 19.916 | 35.777 | 1.00 | 15.78 | | A | O |
| ANISOU | 420 | OG1 | THR | A | 445 | 2033 | 1974 | 1989 | −69 | −16 | 55 | A | O |
| ATOM | 422 | CG2 | THR | A | 445 | −10.566 | 19.197 | 36.721 | 1.00 | 14.65 | | A | C |
| ANISOU | 422 | CG2 | THR | A | 445 | 1946 | 1852 | 1768 | 10 | 14 | 41 | A | C |
| ATOM | 426 | C | THR | A | 445 | −10.892 | 17.475 | 34.117 | 1.00 | 15.56 | | A | C |
| ANISOU | 426 | C | THR | A | 445 | 1926 | 2046 | 1936 | 0 | 23 | 72 | A | C |
| ATOM | 427 | O | THR | A | 445 | −11.581 | 18.169 | 33.379 | 1.00 | 15.80 | | A | O |
| ANISOU | 427 | O | THR | A | 445 | 1908 | 2128 | 1966 | 30 | 45 | 188 | A | O |
| ATOM | 428 | N | ASN | A | 446 | −11.225 | 16.237 | 34.441 | 1.00 | 16.06 | | A | N |
| ANISOU | 428 | N | ASN | A | 446 | 2074 | 2069 | 1957 | −13 | 24 | 44 | A | N |
| ATOM | 430 | CA | ASN | A | 446 | −12.499 | 15.684 | 34.022 | 1.00 | 16.90 | | A | C |
| ANISOU | 430 | CA | ASN | A | 446 | 2187 | 2180 | 2054 | −42 | −2 | 24 | A | C |
| ATOM | 432 | CB | ASN | A | 446 | −12.425 | 14.151 | 33.997 | 1.00 | 16.14 | | A | C |
| ANISOU | 432 | CB | ASN | A | 446 | 2134 | 2071 | 1925 | −31 | 23 | 36 | A | C |
| ATOM | 435 | CG | ASN | A | 446 | −12.366 | 13.525 | 35.395 | 1.00 | 15.76 | | A | C |
| ANISOU | 435 | CG | ASN | A | 446 | 2058 | 2019 | 1908 | −28 | 12 | −3 | A | C |
| ATOM | 436 | OD1 | ASN | A | 446 | −12.610 | 14.174 | 36.394 | 1.00 | 13.92 | | A | O |
| ANISOU | 436 | OD1 | ASN | A | 446 | 1872 | 1816 | 1598 | −97 | −66 | 137 | A | O |
| ATOM | 437 | ND2 | ASN | A | 446 | −12.059 | 12.255 | 35.442 | 1.00 | 16.63 | | A | N |
| ANISOU | 437 | ND2 | ASN | A | 446 | 2320 | 2031 | 1967 | −36 | −35 | 113 | A | N |
| ATOM | 440 | C | ASN | A | 446 | −13.662 | 16.235 | 34.871 | 1.00 | 18.01 | | A | C |
| ANISOU | 440 | C | ASN | A | 446 | 2317 | 2317 | 2205 | −35 | 36 | 5 | A | C |
| ATOM | 441 | O | ASN | A | 446 | −13.461 | 17.105 | 35.722 | 1.00 | 17.82 | | A | O |
| ANISOU | 441 | O | ASN | A | 446 | 2312 | 2331 | 2128 | −126 | 64 | 22 | A | O |
| ATOM | 442 | N | HIS | A | 447 | −14.882 | 15.778 | 34.606 | 1.00 | 19.72 | | A | N |
| ANISOU | 442 | N | HIS | A | 447 | 2522 | 2530 | 2441 | −64 | −23 | −8 | A | N |
| ATOM | 444 | CA | HIS | A | 447 | −16.070 | 16.349 | 35.251 | 1.00 | 21.09 | | A | C |
| ANISOU | 444 | CA | HIS | A | 447 | 2665 | 2717 | 2628 | −16 | −5 | 20 | A | C |
| ATOM | 446 | CB | HIS | A | 447 | −17.347 | 15.890 | 34.530 | 1.00 | 22.10 | | A | C |
| ANISOU | 446 | CB | HIS | A | 447 | 2780 | 2841 | 2775 | −49 | −29 | −18 | A | C |
| ATOM | 449 | CG | HIS | A | 447 | −17.554 | 16.527 | 33.187 | 1.00 | 25.24 | | A | C |
| ANISOU | 449 | CG | HIS | A | 447 | 3287 | 3216 | 3084 | 19 | −31 | 71 | A | C |
| ATOM | 450 | ND1 | HIS | A | 447 | −18.753 | 16.448 | 32.507 | 1.00 | 28.07 | | A | N |
| ANISOU | 450 | ND1 | HIS | A | 447 | 3513 | 3631 | 3517 | −67 | −110 | 37 | A | N |
| ATOM | 452 | CE1 | HIS | A | 447 | −18.647 | 17.092 | 31.357 | 1.00 | 29.07 | | A | C |
| ANISOU | 452 | CE1 | HIS | A | 447 | 3675 | 3756 | 3613 | −61 | −32 | 65 | A | C |
| ATOM | 454 | NE2 | HIS | A | 447 | −17.425 | 17.586 | 31.267 | 1.00 | 28.72 | | A | N |
| ANISOU | 454 | NE2 | HIS | A | 447 | 3615 | 3729 | 3566 | −38 | −30 | 64 | A | N |
| ATOM | 456 | CD2 | HIS | A | 447 | −16.720 | 17.244 | 32.397 | 1.00 | 27.27 | | A | C |
| ANISOU | 456 | CD2 | HIS | A | 447 | 3427 | 3467 | 3464 | −61 | 10 | 69 | A | C |
| ATOM | 458 | C | HIS | A | 447 | −16.170 | 16.022 | 36.758 | 1.00 | 20.82 | | A | C |
| ANISOU | 458 | C | HIS | A | 447 | 2634 | 2677 | 2598 | −14 | −13 | −1 | A | C |
| ATOM | 459 | O | HIS | A | 447 | −16.936 | 16.668 | 37.496 | 1.00 | 21.25 | | A | O |
| ANISOU | 459 | O | HIS | A | 447 | 2642 | 2781 | 2649 | −12 | −47 | 19 | A | O |
| ATOM | 460 | N | LYS | A | 448 | −15.400 | 15.022 | 37.192 | 1.00 | 20.49 | | A | N |
| ANISOU | 460 | N | LYS | A | 448 | 2626 | 2626 | 2530 | −48 | −32 | 41 | A | N |
| ATOM | 462 | CA | LYS | A | 448 | −15.304 | 14.653 | 38.608 | 1.00 | 20.17 | | A | C |
| ANISOU | 462 | CA | LYS | A | 448 | 2586 | 2580 | 2496 | −44 | −26 | 4 | A | C |
| ATOM | 464 | CB | LYS | A | 448 | −15.058 | 13.150 | 38.734 | 1.00 | 20.12 | | A | C |
| ANISOU | 464 | CB | LYS | A | 448 | 2596 | 2562 | 2485 | −16 | −16 | 21 | A | C |
| ATOM | 467 | CG | LYS | A | 448 | −16.243 | 12.328 | 38.264 | 1.00 | 20.68 | | A | C |
| ANISOU | 467 | CG | LYS | A | 448 | 2613 | 2656 | 2586 | −31 | −30 | 0 | A | C |
| ATOM | 470 | CD | LYS | A | 448 | −15.881 | 10.871 | 38.151 | 1.00 | 21.62 | | A | C |
| ANISOU | 470 | CD | LYS | A | 448 | 2803 | 2688 | 2721 | −50 | −67 | 63 | A | C |
| ATOM | 473 | CE | LYS | A | 448 | −16.979 | 10.064 | 37.483 | 1.00 | 21.84 | | A | C |
| ANISOU | 473 | CE | LYS | A | 448 | 2806 | 2767 | 2725 | −25 | −36 | 5 | A | C |
| ATOM | 476 | NZ | LYS | A | 448 | −16.595 | 8.631 | 37.433 | 1.00 | 21.44 | | A | N |
| ANISOU | 476 | NZ | LYS | A | 448 | 2818 | 2691 | 2636 | −26 | −64 | 64 | A | N |
| ATOM | 480 | C | LYS | A | 448 | −14.212 | 15.416 | 39.367 | 1.00 | 19.90 | | A | C |
| ANISOU | 480 | C | LYS | A | 448 | 2560 | 2556 | 2443 | −71 | −18 | 29 | A | C |
| ATOM | 481 | O | LYS | A | 448 | −14.055 | 15.234 | 40.581 | 1.00 | 20.18 | | A | O |
| ANISOU | 481 | O | LYS | A | 448 | 2647 | 2614 | 2407 | −120 | −37 | 45 | A | O |
| ATOM | 482 | N | GLY | A | 449 | −13.466 | 16.261 | 38.660 | 1.00 | 18.93 | | A | N |
| ANISOU | 482 | N | GLY | A | 449 | 2429 | 2436 | 2325 | −48 | −23 | 16 | A | N |
| ATOM | 484 | CA | GLY | A | 449 | −12.438 | 17.082 | 39.268 | 1.00 | 18.33 | | A | C |
| ANISOU | 484 | CA | GLY | A | 449 | 2333 | 2359 | 2270 | −3 | 22 | 17 | A | C |
| ATOM | 487 | C | GLY | A | 449 | −11.099 | 16.384 | 39.325 | 1.00 | 18.11 | | A | C |
| ANISOU | 487 | C | GLY | A | 449 | 2304 | 2334 | 2243 | −10 | 11 | 25 | A | C |
| ATOM | 488 | O | GLY | A | 449 | −10.153 | 16.929 | 39.872 | 1.00 | 18.44 | | A | O |
| ANISOU | 488 | O | GLY | A | 449 | 2343 | 2383 | 2280 | −17 | −13 | 19 | A | O |
| ATOM | 489 | N | GLU | A | 450 | −11.015 | 15.186 | 38.758 | 1.00 | 17.86 | | A | N |
| ANISOU | 489 | N | GLU | A | 450 | 2254 | 2294 | 2235 | −21 | −6 | 48 | A | N |
| ATOM | 491 | CA | GLU | A | 450 | −9.760 | 14.439 | 38.722 | 1.00 | 18.01 | | A | C |
| ANISOU | 491 | CA | GLU | A | 450 | 2280 | 2315 | 2249 | −18 | 22 | 15 | A | C |
| ATOM | 493 | CB | GLU | A | 450 | −10.031 | 12.991 | 38.356 | 1.00 | 18.11 | | A | C |
| ANISOU | 493 | CB | GLU | A | 450 | 2303 | 2300 | 2276 | −2 | −3 | 4 | A | C |
| ATOM | 496 | CG | GLU | A | 450 | −10.952 | 12.287 | 39.340 | 1.00 | 19.27 | | A | C |
| ANISOU | 496 | CG | GLU | A | 450 | 2475 | 2472 | 2374 | −45 | −5 | 60 | A | C |
| ATOM | 499 | CD | GLU | A | 450 | −11.219 | 10.857 | 38.943 | 1.00 | 21.94 | | A | C |
| ANISOU | 499 | CD | GLU | A | 450 | 2934 | 2634 | 2768 | −79 | −38 | 20 | A | C |
| ATOM | 500 | OE1 | GLU | A | 450 | −11.672 | 10.634 | 37.795 | 1.00 | 23.68 | | A | O |
| ANISOU | 500 | OE1 | GLU | A | 450 | 3178 | 2995 | 2822 | −111 | −66 | 77 | A | O |
| ATOM | 501 | OE2 | GLU | A | 450 | −10.962 | 9.957 | 39.774 | 1.00 | 22.58 | | A | O |
| ANISOU | 501 | OE2 | GLU | A | 450 | 3007 | 2808 | 2765 | −104 | −108 | 137 | A | O |
| ATOM | 502 | C | GLU | A | 450 | −8.799 | 15.046 | 37.713 | 1.00 | 18.31 | | A | C |
| ANISOU | 502 | C | GLU | A | 450 | 2319 | 2346 | 2292 | −46 | −2 | 50 | A | C |
| ATOM | 503 | O | GLU | A | 450 | −9.191 | 15.323 | 36.588 | 1.00 | 17.45 | | A | O |
| ANISOU | 503 | O | GLU | A | 450 | 2142 | 2295 | 2192 | −73 | 9 | 49 | A | O |
| ATOM | 504 | N | LYS | A | 451 | −7.542 | 15.226 | 38.122 | 1.00 | 18.88 | | A | N |
| ANISOU | 504 | N | LYS | A | 451 | 2349 | 2450 | 2374 | −27 | 3 | 49 | A | N |
| ATOM | 506 | CA | LYS | A | 451 | −6.537 | 15.886 | 37.292 | 1.00 | 19.30 | | A | C |
| ANISOU | 506 | CA | LYS | A | 451 | 2420 | 2474 | 2436 | −11 | 8 | 41 | A | C |
| ATOM | 508 | CB | LYS | A | 451 | −5.773 | 16.933 | 38.101 | 1.00 | 19.90 | | A | C |
| ANISOU | 508 | CB | LYS | A | 451 | 2504 | 2553 | 2503 | −25 | 0 | 25 | A | C |
| ATOM | 511 | CG | LYS | A | 451 | −6.656 | 18.007 | 38.734 | 1.00 | 21.63 | | A | C |
| ANISOU | 511 | CG | LYS | A | 451 | 2709 | 2729 | 2779 | 2 | 48 | −12 | A | C |
| ATOM | 514 | CD | LYS | A | 451 | −6.191 | 19.431 | 38.405 | 1.00 | 23.25 | | A | C |
| ANISOU | 514 | CD | LYS | A | 451 | 2959 | 2905 | 2968 | −52 | 0 | 53 | A | C |
| ATOM | 517 | CE | LYS | A | 451 | −7.065 | 20.474 | 39.069 | 1.00 | 23.83 | | A | C |
| ANISOU | 517 | CE | LYS | A | 451 | 2991 | 3052 | 3009 | −15 | −6 | 5 | A | C |
| ATOM | 520 | NZ | LYS | A | 451 | −7.846 | 21.294 | 38.116 | 1.00 | 24.29 | | A | N |
| ANISOU | 520 | NZ | LYS | A | 451 | 3103 | 3063 | 3062 | −44 | 7 | 63 | A | N |
| ATOM | 524 | C | LYS | A | 451 | −5.576 | 14.860 | 36.707 | 1.00 | 19.37 | | A | C |
| ANISOU | 524 | C | LYS | A | 451 | 2436 | 2512 | 2411 | −9 | 1 | 43 | A | C |
| ATOM | 525 | O | LYS | A | 451 | −4.954 | 14.066 | 37.431 | 1.00 | 19.54 | | A | O |
| ANISOU | 525 | O | LYS | A | 451 | 2436 | 2534 | 2454 | 17 | −16 | 87 | A | O |
| ATOM | 526 | N | ILE | A | 452 | −5.470 | 14.881 | 35.383 | 1.00 | 19.06 | | A | N |
| ANISOU | 526 | N | ILE | A | 452 | 2411 | 2463 | 2365 | −24 | 21 | 50 | A | N |
| ATOM | 528 | CA | ILE | A | 452 | −4.694 | 13.928 | 34.622 | 1.00 | 19.44 | | A | C |
| ANISOU | 528 | CA | ILE | A | 452 | 2448 | 2471 | 2464 | −14 | 8 | 59 | A | C |
| ATOM | 530 | CB | ILE | A | 452 | −5.684 | 13.140 | 33.686 | 1.00 | 20.36 | | A | C |
| ANISOU | 530 | CB | ILE | A | 452 | 2588 | 2603 | 2544 | −48 | −29 | 27 | A | C |
| ATOM | 532 | CG1 | ILE | A | 452 | −6.674 | 12.316 | 34.541 | 1.00 | 22.71 | | A | C |
| ANISOU | 532 | CG1 | ILE | A | 452 | 2879 | 2905 | 2845 | −80 | 23 | 68 | A | C |
| ATOM | 535 | CD1 | ILE | A | 452 | −7.858 | 11.768 | 33.782 | 1.00 | 24.16 | | A | C |
| ANISOU | 535 | CD1 | ILE | A | 452 | 3037 | 3086 | 3054 | −90 | −39 | 50 | A | C |
| ATOM | 539 | CG2 | ILE | A | 452 | −4.975 | 12.233 | 32.713 | 1.00 | 20.63 | | A | C |
| ANISOU | 539 | CG2 | ILE | A | 452 | 2573 | 2651 | 2614 | −15 | −20 | 36 | A | C |
| ATOM | 543 | C | ILE | A | 452 | −3.654 | 14.733 | 33.834 | 1.00 | 17.96 | | A | C |
| ANISOU | 543 | C | ILE | A | 452 | 2251 | 2313 | 2261 | 47 | 8 | 55 | A | C |
| ATOM | 544 | O | ILE | A | 452 | −4.010 | 15.652 | 33.113 | 1.00 | 17.20 | | A | O |
| ANISOU | 544 | O | ILE | A | 452 | 2194 | 2146 | 2193 | 131 | 84 | 90 | A | O |
| ATOM | 545 | N | ASN | A | 453 | −2.371 | 14.422 | 33.996 | 1.00 | 16.81 | | A | N |
| ANISOU | 545 | N | ASN | A | 453 | 2139 | 2108 | 2137 | 76 | −9 | 45 | A | N |
| ATOM | 547 | CA | ASN | A | 453 | −1.331 | 15.050 | 33.194 | 1.00 | 16.18 | | A | C |
| ANISOU | 547 | CA | ASN | A | 453 | 2022 | 2087 | 2036 | 66 | −52 | 18 | A | C |
| ATOM | 549 | CB | ASN | A | 453 | 0.070 | 14.751 | 33.747 | 1.00 | 17.02 | | A | C |
| ANISOU | 549 | CB | ASN | A | 453 | 2092 | 2199 | 2173 | 85 | −58 | 36 | A | C |
| ATOM | 552 | CG | ASN | A | 453 | 0.234 | 15.222 | 35.166 | 1.00 | 19.63 | | A | C |
| ANISOU | 552 | CG | ASN | A | 453 | 2501 | 2585 | 2370 | 9 | −62 | −41 | A | C |
| ATOM | 553 | OD1 | ASN | A | 453 | 0.683 | 14.462 | 36.040 | 1.00 | 24.36 | | A | O |
| ANISOU | 553 | OD1 | ASN | A | 453 | 3128 | 3251 | 2877 | 94 | −220 | 164 | A | O |
| ATOM | 554 | ND2 | ASN | A | 453 | −0.154 | 16.457 | 35.421 | 1.00 | 18.71 | | A | N |
| ANISOU | 554 | ND2 | ASN | A | 453 | 2282 | 2542 | 2281 | 10 | −178 | 18 | A | N |
| ATOM | 557 | C | ASN | A | 453 | −1.415 | 14.572 | 31.758 | 1.00 | 14.71 | | A | C |
| ANISOU | 557 | C | ASN | A | 453 | 1820 | 1874 | 1895 | 74 | −27 | 46 | A | C |
| ATOM | 558 | O | ASN | A | 453 | −1.539 | 13.379 | 31.497 | 1.00 | 15.20 | | A | O |
| ANISOU | 558 | O | ASN | A | 453 | 1937 | 1971 | 1865 | 86 | −101 | 37 | A | O |
| ATOM | 559 | N | VAL | A | 454 | −1.367 | 15.525 | 30.836 | 1.00 | 12.89 | | A | N |
| ANISOU | 559 | N | VAL | A | 454 | 1613 | 1593 | 1688 | 50 | −48 | 0 | A | N |
| ATOM | 561 | CA | VAL | A | 454 | −1.511 | 15.246 | 29.418 | 1.00 | 11.46 | | A | C |
| ANISOU | 561 | CA | VAL | A | 454 | 1412 | 1412 | 1528 | 60 | −41 | 23 | A | C |
| ATOM | 563 | CB | VAL | A | 454 | −2.910 | 15.649 | 28.880 | 1.00 | 10.74 | | A | C |
| ANISOU | 563 | CB | VAL | A | 454 | 1336 | 1273 | 1468 | 40 | −43 | 14 | A | C |
| ATOM | 565 | CG1 | VAL | A | 454 | −3.991 | 14.806 | 29.534 | 1.00 | 11.37 | | A | C |
| ANISOU | 565 | CG1 | VAL | A | 454 | 1323 | 1411 | 1587 | 82 | −8 | 21 | A | C |
| ATOM | 569 | CG2 | VAL | A | 454 | −3.191 | 17.144 | 29.047 | 1.00 | 10.27 | | A | C |
| ANISOU | 569 | CG2 | VAL | A | 454 | 1352 | 1251 | 1296 | 24 | −24 | −6 | A | C |
| ATOM | 573 | C | VAL | A | 454 | −0.432 | 15.913 | 28.586 | 1.00 | 11.10 | | A | C |
| ANISOU | 573 | C | VAL | A | 454 | 1375 | 1356 | 1487 | 28 | −75 | 9 | A | C |
| ATOM | 574 | O | VAL | A | 454 | 0.158 | 16.934 | 28.982 | 1.00 | 11.62 | | A | O |
| ANISOU | 574 | O | VAL | A | 454 | 1341 | 1411 | 1664 | 32 | −123 | −108 | A | O |
| ATOM | 575 | N | ALA | A | 455 | −0.164 | 15.290 | 27.444 | 1.00 | 10.28 | | A | N |
| ANISOU | 575 | N | ALA | A | 455 | 1293 | 1177 | 1436 | 48 | −86 | −43 | A | N |
| ATOM | 577 | CA | ALA | A | 455 | 0.601 | 15.841 | 26.361 | 1.00 | 10.79 | | A | C |
| ANISOU | 577 | CA | ALA | A | 455 | 1369 | 1251 | 1478 | −37 | −90 | 8 | A | C |
| ATOM | 579 | CB | ALA | A | 455 | 1.607 | 14.858 | 25.836 | 1.00 | 10.88 | | A | C |
| ANISOU | 579 | CB | ALA | A | 455 | 1292 | 1424 | 1418 | −44 | 25 | 27 | A | C |
| ATOM | 583 | C | ALA | A | 455 | −0.407 | 16.225 | 25.275 | 1.00 | 11.22 | | A | C |
| ANISOU | 583 | C | ALA | A | 455 | 1454 | 1377 | 1432 | −102 | −122 | 32 | A | C |
| ATOM | 584 | O | ALA | A | 455 | −1.261 | 15.418 | 24.872 | 1.00 | 13.07 | | A | O |
| ANISOU | 584 | O | ALA | A | 455 | 1746 | 1421 | 1799 | −243 | −207 | 112 | A | O |
| ATOM | 585 | N | VAL | A | 456 | −0.345 | 17.468 | 24.840 | 1.00 | 10.13 | | A | N |
| ANISOU | 585 | N | VAL | A | 456 | 1323 | 1231 | 1293 | −95 | −15 | 0 | A | N |
| ATOM | 587 | CA | VAL | A | 456 | −1.261 | 17.960 | 23.835 | 1.00 | 10.58 | | A | C |
| ANISOU | 587 | CA | VAL | A | 456 | 1347 | 1340 | 1331 | −21 | −8 | −11 | A | C |
| ATOM | 589 | CB | VAL | A | 456 | −2.037 | 19.199 | 24.300 | 1.00 | 9.96 | | A | C |
| ANISOU | 589 | CB | VAL | A | 456 | 1317 | 1285 | 1181 | −26 | −1 | −13 | A | C |
| ATOM | 591 | CG1 | VAL | A | 456 | −3.004 | 19.641 | 23.192 | 1.00 | 11.36 | | A | C |
| ANISOU | 591 | CG1 | VAL | A | 456 | 1448 | 1516 | 1350 | 42 | −66 | −1 | A | C |
| ATOM | 595 | CG2 | VAL | A | 456 | −2.804 | 18.879 | 25.581 | 1.00 | 11.32 | | A | C |
| ANISOU | 595 | CG2 | VAL | A | 456 | 1438 | 1590 | 1271 | −62 | 18 | 3 | A | C |
| ATOM | 599 | C | VAL | A | 456 | −0.521 | 18.262 | 22.553 | 1.00 | 11.04 | | A | C |
| ANISOU | 599 | C | VAL | A | 456 | 1483 | 1337 | 1373 | −99 | 4 | 20 | A | C |
| ATOM | 600 | O | VAL | A | 456 | 0.326 | 19.144 | 22.517 | 1.00 | 11.55 | | A | O |
| ANISOU | 600 | O | VAL | A | 456 | 1503 | 1375 | 1511 | −173 | 61 | −40 | A | O |
| ATOM | 601 | N | LYS | A | 457 | −0.828 | 17.505 | 21.501 | 1.00 | 10.92 | | A | N |
| ANISOU | 601 | N | LYS | A | 457 | 1460 | 1352 | 1337 | −155 | −12 | −36 | A | N |
| ATOM | 603 | CA | LYS | A | 457 | −0.189 | 17.668 | 20.205 | 1.00 | 12.29 | | A | C |
| ANISOU | 603 | CA | LYS | A | 457 | 1578 | 1564 | 1526 | −86 | 13 | −46 | A | C |
| ATOM | 605 | CB | LYS | A | 457 | −0.165 | 16.343 | 19.447 | 1.00 | 12.94 | | A | C |
| ANISOU | 605 | CB | LYS | A | 457 | 1646 | 1617 | 1650 | −78 | 16 | −121 | A | C |
| ATOM | 608 | CG | LYS | A | 457 | 0.446 | 15.205 | 20.233 | 1.00 | 17.77 | | A | C |
| ANISOU | 608 | CG | LYS | A | 457 | 2283 | 2217 | 2251 | 51 | −21 | 73 | A | C |
| ATOM | 611 | CD | LYS | A | 457 | 0.979 | 14.101 | 19.312 | 1.00 | 22.35 | | A | C |
| ANISOU | 611 | CD | LYS | A | 457 | 2962 | 2729 | 2800 | 108 | 29 | −131 | A | C |
| ATOM | 614 | CE | LYS | A | 457 | −0.007 | 12.979 | 19.123 | 1.00 | 25.98 | | A | C |
| ANISOU | 614 | CE | LYS | A | 457 | 3284 | 3293 | 3291 | −78 | 1 | 2 | A | C |
| ATOM | 617 | NZ | LYS | A | 457 | 0.634 | 11.893 | 18.327 | 1.00 | 28.65 | | A | N |
| ANISOU | 617 | NZ | LYS | A | 457 | 3675 | 3488 | 3720 | 123 | 12 | −70 | A | N |
| ATOM | 621 | C | LYS | A | 457 | −0.959 | 18.664 | 19.387 | 1.00 | 11.53 | | A | C |
| ANISOU | 621 | C | LYS | A | 457 | 1538 | 1480 | 1363 | −102 | 17 | −71 | A | C |
| ATOM | 622 | O | LYS | A | 457 | −2.186 | 18.621 | 19.333 | 1.00 | 11.18 | | A | O |
| ANISOU | 622 | O | LYS | A | 457 | 1602 | 1472 | 1174 | −173 | −41 | −49 | A | O |
| ATOM | 623 | N | THR | A | 458 | −0.228 | 19.579 | 18.763 | 1.00 | 11.68 | | A | N |
| ANISOU | 623 | N | THR | A | 458 | 1544 | 1514 | 1379 | −112 | 59 | −67 | A | N |
| ATOM | 625 | CA | THR | A | 458 | −0.809 | 20.529 | 17.844 | 1.00 | 11.93 | | A | C |
| ANISOU | 625 | CA | THR | A | 458 | 1562 | 1549 | 1422 | −55 | 13 | −86 | A | C |
| ATOM | 627 | CB | THR | A | 458 | −0.725 | 21.966 | 18.383 | 1.00 | 12.32 | | A | C |
| ANISOU | 627 | CB | THR | A | 458 | 1602 | 1575 | 1502 | −45 | 27 | −84 | A | C |
| ATOM | 629 | OG1 | THR | A | 458 | 0.647 | 22.381 | 18.478 | 1.00 | 11.89 | | A | O |
| ANISOU | 629 | OG1 | THR | A | 458 | 1577 | 1768 | 1173 | −132 | 119 | −164 | A | O |
| ATOM | 631 | CG2 | THR | A | 458 | −1.281 | 22.017 | 19.799 | 1.00 | 10.96 | | A | C |
| ANISOU | 631 | CG2 | THR | A | 458 | 1506 | 1256 | 1402 | −84 | −12 | −48 | A | C |
| ATOM | 635 | C | THR | A | 458 | −0.114 | 20.446 | 16.505 | 1.00 | 13.06 | | A | C |
| ANISOU | 635 | C | THR | A | 458 | 1684 | 1720 | 1558 | −50 | 23 | −88 | A | C |
| ATOM | 636 | O | THR | A | 458 | 0.942 | 19.832 | 16.386 | 1.00 | 12.37 | | A | O |
| ANISOU | 636 | O | THR | A | 458 | 1679 | 1806 | 1213 | −53 | 111 | −209 | A | O |
| ATOM | 637 | N | CYS | A | 459 | −0.721 | 21.088 | 15.521 | 1.00 | 14.61 | | A | N |
| ANISOU | 637 | N | CYS | A | 459 | 1964 | 1845 | 1742 | −44 | 0 | −64 | A | N |
| ATOM | 639 | CA | CYS | A | 459 | −0.188 | 21.077 | 14.165 | 1.00 | 15.99 | | A | C |
| ANISOU | 639 | CA | CYS | A | 459 | 2103 | 2069 | 1903 | −100 | 10 | −46 | A | C |
| ATOM | 641 | CB | CYS | A | 459 | −1.264 | 20.724 | 13.138 | 1.00 | 17.53 | | A | C |
| ANISOU | 641 | CB | CYS | A | 459 | 2227 | 2423 | 2008 | −37 | −47 | −39 | A | C |
| ATOM | 644 | SG | CYS | A | 459 | −1.594 | 18.929 | 13.033 | 1.00 | 22.86 | | A | S |
| ANISOU | 644 | SG | CYS | A | 459 | 2952 | 3255 | 2478 | −791 | −155 | −532 | A | S |
| ATOM | 645 | C | CYS | A | 459 | 0.429 | 22.421 | 13.871 | 1.00 | 15.76 | | A | C |
| ANISOU | 645 | C | CYS | A | 459 | 2067 | 2029 | 1890 | −33 | −49 | −29 | A | C |
| ATOM | 646 | O | CYS | A | 459 | −0.171 | 23.463 | 14.136 | 1.00 | 14.36 | | A | O |
| ANISOU | 646 | O | CYS | A | 459 | 1973 | 1890 | 1590 | −69 | −114 | −87 | A | O |
| ATOM | 647 | N | LYS | A | 460 | 1.645 | 22.365 | 13.349 | 1.00 | 15.95 | | A | N |
| ANISOU | 647 | N | LYS | A | 460 | 2113 | 2056 | 1890 | −9 | −41 | −59 | A | N |
| ATOM | 649 | CA | LYS | A | 460 | 2.361 | 23.536 | 12.911 | 1.00 | 17.02 | | A | C |
| ANISOU | 649 | CA | LYS | A | 460 | 2198 | 2157 | 2112 | −48 | −11 | −38 | A | C |
| ATOM | 651 | CB | LYS | A | 460 | 3.765 | 23.181 | 12.424 | 1.00 | 17.45 | | A | C |
| ANISOU | 651 | CB | LYS | A | 460 | 2237 | 2213 | 2179 | −27 | 7 | 7 | A | C |
| ATOM | 654 | CG | LYS | A | 460 | 3.838 | 22.242 | 11.232 | 1.00 | 20.13 | | A | C |
| ANISOU | 654 | CG | LYS | A | 460 | 2645 | 2538 | 2462 | −36 | −7 | −72 | A | C |
| ATOM | 657 | CD | LYS | A | 460 | 5.301 | 21.915 | 10.910 | 1.00 | 23.80 | | A | C |
| ANISOU | 657 | CD | LYS | A | 460 | 2908 | 3102 | 3031 | 26 | 48 | −61 | A | C |
| ATOM | 660 | CE | LYS | A | 460 | 5.857 | 20.843 | 11.829 | 1.00 | 25.78 | | A | C |
| ANISOU | 660 | CE | LYS | A | 460 | 3269 | 3291 | 3234 | 4 | −31 | 8 | A | C |
| ATOM | 663 | NZ | LYS | A | 460 | 7.303 | 20.538 | 11.561 | 1.00 | 27.95 | | A | N |
| ANISOU | 663 | NZ | LYS | A | 460 | 3409 | 3621 | 3590 | 52 | 3 | −7 | A | N |
| ATOM | 667 | C | LYS | A | 460 | 1.574 | 24.265 | 11.847 | 1.00 | 16.99 | | A | C |
| ANISOU | 667 | C | LYS | A | 460 | 2162 | 2160 | 2132 | −34 | −19 | −27 | A | C |
| ATOM | 668 | O | LYS | A | 460 | 0.721 | 23.670 | 11.157 | 1.00 | 17.16 | | A | O |
| ANISOU | 668 | O | LYS | A | 460 | 2149 | 2281 | 2089 | −98 | −50 | −66 | A | O |
| ATOM | 669 | N | LYS | A | 461 | 1.837 | 25.561 | 11.719 | 1.00 | 17.39 | | A | N |
| ANISOU | 669 | N | LYS | A | 461 | 2225 | 2204 | 2175 | −47 | −19 | −52 | A | N |
| ATOM | 671 | CA | LYS | A | 461 | 1.047 | 26.390 | 10.833 | 1.00 | 17.93 | | A | C |
| ANISOU | 671 | CA | LYS | A | 461 | 2281 | 2258 | 2273 | −28 | −23 | −8 | A | C |
| ATOM | 673 | CB | LYS | A | 461 | 1.414 | 27.867 | 10.985 | 1.00 | 18.42 | | A | C |
| ANISOU | 673 | CB | LYS | A | 461 | 2355 | 2350 | 2292 | 0 | −8 | −15 | A | C |
| ATOM | 676 | CG | LYS | A | 461 | 2.717 | 28.200 | 10.442 | 1.00 | 18.49 | | A | C |
| ANISOU | 676 | CG | LYS | A | 461 | 2340 | 2328 | 2358 | −46 | −45 | 7 | A | C |
| ATOM | 679 | CD | LYS | A | 461 | 3.000 | 29.687 | 10.608 | 1.00 | 17.75 | | A | C |
| ANISOU | 679 | CD | LYS | A | 461 | 2247 | 2259 | 2239 | −72 | −2 | 8 | A | C |
| ATOM | 682 | CE | LYS | A | 461 | 4.327 | 29.964 | 10.021 | 1.00 | 17.68 | | A | C |
| ANISOU | 682 | CE | LYS | A | 461 | 2220 | 2316 | 2182 | 34 | 12 | −2 | A | C |
| ATOM | 685 | NZ | LYS | A | 461 | 4.595 | 31.378 | 9.966 | 1.00 | 15.93 | | A | N |
| ANISOU | 685 | NZ | LYS | A | 461 | 1954 | 2170 | 1928 | −119 | 95 | −100 | A | N |
| ATOM | 689 | C | LYS | A | 461 | 1.175 | 25.903 | 9.392 | 1.00 | 18.50 | | A | C |
| ANISOU | 689 | C | LYS | A | 461 | 2362 | 2352 | 2315 | −26 | 11 | 16 | A | C |
| ATOM | 690 | O | LYS | A | 461 | 0.235 | 26.036 | 8.618 | 1.00 | 18.56 | | A | O |
| ANISOU | 690 | O | LYS | A | 461 | 2350 | 2395 | 2305 | −37 | −11 | 8 | A | O |
| ATOM | 691 | N | ASP | A | 462 | 2.309 | 25.285 | 9.060 | 1.00 | 19.44 | | A | N |
| ANISOU | 691 | N | ASP | A | 462 | 2459 | 2469 | 2457 | −34 | 0 | 5 | A | N |
| ATOM | 693 | CA | ASP | A | 462 | 2.521 | 24.705 | 7.737 | 1.00 | 20.50 | | A | C |
| ANISOU | 693 | CA | ASP | A | 462 | 2615 | 2583 | 2591 | −22 | −2 | −34 | A | C |
| ATOM | 695 | CB | ASP | A | 462 | 4.011 | 24.747 | 7.340 | 1.00 | 21.57 | | A | C |
| ANISOU | 695 | CB | ASP | A | 462 | 2697 | 2744 | 2751 | −6 | 2 | −44 | A | C |
| ATOM | 698 | CG | ASP | A | 462 | 4.234 | 24.606 | 5.838 | 1.00 | 24.85 | | A | C |
| ANISOU | 698 | CG | ASP | A | 462 | 3199 | 3201 | 3038 | −17 | 17 | −52 | A | C |
| ATOM | 699 | OD1 | ASP | A | 462 | 3.548 | 25.288 | 5.060 | 1.00 | 28.28 | | A | O |
| ANISOU | 699 | OD1 | ASP | A | 462 | 3690 | 3668 | 3386 | 85 | −51 | 39 | A | O |
| ATOM | 700 | OD2 | ASP | A | 462 | 5.087 | 23.840 | 5.340 | 1.00 | 28.72 | | A | O |
| ANISOU | 700 | OD2 | ASP | A | 462 | 3522 | 3761 | 3627 | 106 | 77 | −169 | A | O |
| ATOM | 701 | C | ASP | A | 462 | 1.984 | 23.267 | 7.746 | 1.00 | 20.27 | | A | C |
| ANISOU | 701 | C | ASP | A | 462 | 2566 | 2573 | 2561 | 0 | −42 | −48 | A | C |
| ATOM | 702 | O | ASP | A | 462 | 2.745 | 22.306 | 7.894 | 1.00 | 22.31 | | A | O |
| ANISOU | 702 | O | ASP | A | 462 | 2814 | 2842 | 2819 | 33 | −30 | −149 | A | O |
| ATOM | 703 | N | CME | A | 463 | 0.709 | 23.148 | 7.402 | 1.00 | 19.42 | | A | N |
| ANISOU | 703 | N | CME | A | 463 | 2456 | 2468 | 2452 | −17 | −24 | −70 | A | N |
| ATOM | 706 | CA | CME | A | 463 | 0.071 | 21.860 | 7.475 | 1.00 | 18.40 | | A | C |
| ANISOU | 706 | CA | CME | A | 463 | 2334 | 2392 | 2264 | −16 | −29 | −36 | A | C |
| ATOM | 708 | CB | CME | A | 463 | −0.254 | 21.485 | 8.939 | 1.00 | 19.10 | | A | C |
| ANISOU | 708 | CB | CME | A | 463 | 2395 | 2503 | 2359 | −7 | −38 | −59 | A | C |
| ATOM | 711 | SG | CME | A | 463 | −0.977 | 19.896 | 9.168 | 1.00 | 23.43 | | A | S |
| ANISOU | 711 | SG | CME | A | 463 | 3199 | 3069 | 2632 | −199 | −114 | −144 | A | S |
| ATOM | 712 | S2 | CME | A | 463 | 0.497 | 18.544 | 9.074 | 1.00 | 28.04 | | A | S |
| ANISOU | 712 | S2 | CME | A | 463 | 3750 | 3133 | 3767 | −94 | −119 | −278 | A | S |
| ATOM | 713 | C2 | CME | A | 463 | 2.017 | 19.054 | 8.364 | 1.00 | 30.33 | | A | C |
| ANISOU | 713 | C2 | CME | A | 463 | 3927 | 3790 | 3807 | −48 | 62 | −51 | A | C |
| ATOM | 716 | C1 | CME | A | 463 | 3.166 | 18.838 | 9.310 | 1.00 | 33.78 | | A | C |
| ANISOU | 716 | C1 | CME | A | 463 | 4277 | 4290 | 4268 | 22 | −49 | −13 | A | C |
| ATOM | 718 | O1 | CME | A | 463 | 4.145 | 19.578 | 9.206 | 1.00 | 36.82 | | A | O |
| ANISOU | 718 | O1 | CME | A | 463 | 4541 | 4709 | 4738 | −68 | 14 | −4 | A | O |
| ATOM | 719 | C | CME | A | 463 | −1.280 | 21.972 | 6.924 | 1.00 | 16.38 | | A | C |
| ANISOU | 719 | C | CME | A | 463 | 2122 | 2097 | 2002 | −23 | 0 | −43 | A | C |
| ATOM | 720 | O | CME | A | 463 | −2.086 | 22.802 | 7.149 | 1.00 | 14.95 | | A | O |
| ANISOU | 720 | O | CME | A | 463 | 2000 | 1969 | 1709 | −105 | −36 | −112 | A | O |
| ATOM | 722 | N | THR | A | 464 | −1.287 | 21.329 | 5.764 | 1.00 | 14.94 | | A | N |
| ANISOU | 722 | N | THR | A | 464 | 1857 | 1986 | 1830 | −7 | −27 | −64 | A | N |
| ATOM | 724 | CA | THR | A | 464 | −2.339 | 21.522 | 4.798 | 1.00 | 14.07 | | A | C |
| ANISOU | 724 | CA | THR | A | 464 | 1784 | 1892 | 1669 | −25 | −5 | −99 | A | C |
| ATOM | 726 | CB | THR | A | 464 | −1.907 | 21.016 | 3.430 | 1.00 | 14.07 | | A | C |
| ANISOU | 726 | CB | THR | A | 464 | 1763 | 1982 | 1601 | −92 | −27 | −89 | A | C |
| ATOM | 728 | OG1 | THR | A | 464 | −1.504 | 19.649 | 3.548 | 1.00 | 13.52 | | A | O |
| ANISOU | 728 | OG1 | THR | A | 464 | 1653 | 2136 | 1348 | −200 | −63 | −217 | A | O |
| ATOM | 730 | CG2 | THR | A | 464 | −0.682 | 21.763 | 2.914 | 1.00 | 15.07 | | A | C |
| ANISOU | 730 | CG2 | THR | A | 464 | 1880 | 2134 | 1712 | −83 | 40 | −130 | A | C |
| ATOM | 734 | C | THR | A | 464 | −3.501 | 20.713 | 5.263 | 1.00 | 13.24 | | A | C |
| ANISOU | 734 | C | THR | A | 464 | 1697 | 1807 | 1525 | 13 | 1 | −117 | A | C |
| ATOM | 735 | O | THR | A | 464 | −3.355 | 19.782 | 6.037 | 1.00 | 12.68 | | A | O |
| ANISOU | 735 | O | THR | A | 464 | 1617 | 1760 | 1440 | −16 | −50 | −274 | A | O |
| ATOM | 736 | N | LEU | A | 465 | −4.673 | 21.066 | 4.767 | 1.00 | 12.73 | | A | N |
| ANISOU | 736 | N | LEU | A | 465 | 1606 | 1808 | 1421 | 15 | −33 | −154 | A | N |
| ATOM | 738 | CA | LEU | A | 465 | −5.853 | 20.278 | 5.063 | 1.00 | 12.88 | | A | C |
| ANISOU | 738 | CA | LEU | A | 465 | 1663 | 1728 | 1500 | −32 | −6 | −57 | A | C |
| ATOM | 740 | CB | LEU | A | 465 | −7.067 | 20.931 | 4.430 | 1.00 | 12.55 | | A | C |
| ANISOU | 740 | CB | LEU | A | 465 | 1625 | 1683 | 1458 | −2 | 5 | −12 | A | C |
| ATOM | 743 | CG | LEU | A | 465 | −7.490 | 22.259 | 5.046 | 1.00 | 12.91 | | A | C |
| ANISOU | 743 | CG | LEU | A | 465 | 1669 | 1664 | 1571 | −86 | 56 | −30 | A | C |
| ATOM | 745 | CD1 | LEU | A | 465 | −8.576 | 22.885 | 4.210 | 1.00 | 15.25 | | A | C |
| ANISOU | 745 | CD1 | LEU | A | 465 | 1932 | 2033 | 1828 | 72 | 18 | −84 | A | C |
| ATOM | 749 | CD2 | LEU | A | 465 | −7.940 | 22.071 | 6.486 | 1.00 | 14.13 | | A | C |
| ANISOU | 749 | CD2 | LEU | A | 465 | 1832 | 1916 | 1621 | −7 | 84 | −88 | A | C |
| ATOM | 753 | C | LEU | A | 465 | −5.713 | 18.851 | 4.555 | 1.00 | 13.30 | | A | C |
| ANISOU | 753 | C | LEU | A | 465 | 1696 | 1784 | 1572 | −58 | −52 | −54 | A | C |
| ATOM | 754 | O | LEU | A | 465 | −6.233 | 17.919 | 5.137 | 1.00 | 13.10 | | A | O |
| ANISOU | 754 | O | LEU | A | 465 | 1757 | 1772 | 1448 | −150 | −106 | −125 | A | O |
| ATOM | 755 | N | ASP | A | 466 | −5.001 | 18.688 | 3.447 | 1.00 | 14.29 | | A | N |
| ANISOU | 755 | N | ASP | A | 466 | 1732 | 1956 | 1740 | −59 | −15 | −103 | A | N |
| ATOM | 757 | CA | ASP | A | 466 | −4.649 | 17.365 | 2.933 | 1.00 | 15.75 | | A | C |
| ANISOU | 757 | CA | ASP | A | 466 | 1964 | 2021 | 1998 | −31 | −13 | −52 | A | C |
| ATOM | 759 | CB | ASP | A | 466 | −3.768 | 17.595 | 1.694 | 1.00 | 16.19 | | A | C |
| ANISOU | 759 | CB | ASP | A | 466 | 2013 | 2092 | 2046 | 1 | 7 | −64 | A | C |
| ATOM | 762 | CG | ASP | A | 466 | −3.400 | 16.334 | 0.957 | 1.00 | 18.38 | | A | C |
| ANISOU | 762 | CG | ASP | A | 466 | 2366 | 2283 | 2335 | 45 | 38 | −63 | A | C |
| ATOM | 763 | OD1 | ASP | A | 466 | −3.566 | 15.220 | 1.482 | 1.00 | 18.27 | | A | O |
| ANISOU | 763 | OD1 | ASP | A | 466 | 2282 | 2225 | 2432 | 137 | 40 | −28 | A | O |
| ATOM | 764 | OD2 | ASP | A | 466 | −2.891 | 16.411 | −0.190 | 1.00 | 21.14 | | A | O |
| ANISOU | 764 | OD2 | ASP | A | 466 | 2704 | 2875 | 2450 | 178 | 57 | −3 | A | O |
| ATOM | 765 | C | ASP | A | 466 | −3.957 | 16.536 | 4.027 | 1.00 | 16.82 | | A | C |
| ANISOU | 765 | C | ASP | A | 466 | 2101 | 2140 | 2146 | 15 | 6 | −71 | A | C |
| ATOM | 766 | O | ASP | A | 466 | −4.444 | 15.491 | 4.438 | 1.00 | 16.81 | | A | O |
| ANISOU | 766 | O | ASP | A | 466 | 1934 | 2293 | 2159 | 0 | 21 | −96 | A | O |
| ATOM | 767 | N | ASN | A | 467 | −2.832 | 17.026 | 4.534 | 1.00 | 18.07 | | A | N |
| ANISOU | 767 | N | ASN | A | 467 | 2214 | 2341 | 2311 | −35 | −11 | −57 | A | N |
| ATOM | 769 | CA | ASN | A | 467 | −2.121 | 16.313 | 5.582 | 1.00 | 19.78 | | A | C |
| ANISOU | 769 | CA | ASN | A | 467 | 2477 | 2498 | 2538 | 5 | −28 | −32 | A | C |
| ATOM | 771 | CB | ASN | A | 467 | −0.737 | 16.928 | 5.794 | 1.00 | 20.56 | | A | C |
| ANISOU | 771 | CB | ASN | A | 467 | 2537 | 2625 | 2648 | −32 | −31 | −20 | A | C |
| ATOM | 774 | CG | ASN | A | 467 | 0.229 | 16.574 | 4.672 | 1.00 | 22.62 | | A | C |
| ANISOU | 774 | CG | ASN | A | 467 | 2775 | 2889 | 2929 | 37 | 68 | −61 | A | C |
| ATOM | 775 | OD1 | ASN | A | 467 | 0.859 | 17.452 | 4.057 | 1.00 | 23.81 | | A | O |
| ANISOU | 775 | OD1 | ASN | A | 467 | 2798 | 2995 | 3252 | 76 | 149 | −58 | A | O |
| ATOM | 776 | ND2 | ASN | A | 467 | 0.331 | 15.278 | 4.381 | 1.00 | 24.98 | | A | N |
| ANISOU | 776 | ND2 | ASN | A | 467 | 3031 | 3041 | 3416 | 68 | 39 | −58 | A | N |
| ATOM | 779 | C | ASN | A | 467 | −2.899 | 16.237 | 6.892 | 1.00 | 20.16 | | A | C |
| ANISOU | 779 | C | ASN | A | 467 | 2520 | 2560 | 2578 | −21 | −41 | −14 | A | C |
| ATOM | 780 | O | ASN | A | 467 | −2.808 | 15.241 | 7.614 | 1.00 | 20.23 | | A | O |
| ANISOU | 780 | O | ASN | A | 467 | 2562 | 2539 | 2583 | −41 | −18 | −24 | A | O |
| ATOM | 781 | N | LYS | A | 468 | −3.681 | 17.266 | 7.201 | 1.00 | 20.54 | | A | N |
| ANISOU | 781 | N | LYS | A | 468 | 2605 | 2564 | 2635 | −26 | −40 | −31 | A | N |
| ATOM | 783 | CA | LYS | A | 468 | −4.477 | 17.259 | 8.422 | 1.00 | 21.30 | | A | C |
| ANISOU | 783 | CA | LYS | A | 468 | 2715 | 2684 | 2695 | −42 | −4 | −4 | A | C |
| ATOM | 785 | CB | LYS | A | 468 | −5.207 | 18.591 | 8.633 | 1.00 | 21.59 | | A | C |
| ANISOU | 785 | CB | LYS | A | 468 | 2728 | 2722 | 2752 | 6 | 5 | 32 | A | C |
| ATOM | 788 | CG | LYS | A | 468 | −4.377 | 19.632 | 9.351 | 1.00 | 24.20 | | A | C |
| ANISOU | 788 | CG | LYS | A | 468 | 3026 | 3103 | 3062 | −27 | −13 | −39 | A | C |
| ATOM | 791 | CD | LYS | A | 468 | −5.128 | 20.926 | 9.566 | 1.00 | 25.91 | | A | C |
| ANISOU | 791 | CD | LYS | A | 468 | 3276 | 3253 | 3313 | 48 | 20 | −28 | A | C |
| ATOM | 794 | CE | LYS | A | 468 | −4.182 | 22.044 | 9.994 | 1.00 | 27.19 | | A | C |
| ANISOU | 794 | CE | LYS | A | 468 | 3404 | 3437 | 3490 | −51 | 3 | −23 | A | C |
| ATOM | 797 | NZ | LYS | A | 468 | −4.900 | 23.299 | 10.354 | 1.00 | 28.40 | | A | N |
| ANISOU | 797 | NZ | LYS | A | 468 | 3622 | 3639 | 3529 | 41 | 41 | −9 | A | N |
| ATOM | 801 | C | LYS | A | 468 | −5.495 | 16.116 | 8.427 | 1.00 | 21.72 | | A | C |
| ANISOU | 801 | C | LYS | A | 468 | 2785 | 2719 | 2748 | −68 | 7 | 22 | A | C |
| ATOM | 802 | O | LYS | A | 468 | −5.729 | 15.526 | 9.476 | 1.00 | 22.45 | | A | O |
| ANISOU | 802 | O | LYS | A | 468 | 2914 | 2767 | 2846 | −79 | −3 | 80 | A | O |
| ATOM | 803 | N | GLU | A | 469 | −6.078 | 15.798 | 7.269 | 1.00 | 21.72 | | A | N |
| ANISOU | 803 | N | GLU | A | 469 | 2775 | 2723 | 2754 | −37 | −26 | 54 | A | N |
| ATOM | 805 | CA | GLU | A | 469 | −7.038 | 14.687 | 7.163 | 1.00 | 22.61 | | A | C |
| ANISOU | 805 | CA | GLU | A | 469 | 2875 | 2844 | 2869 | −47 | −9 | 16 | A | C |
| ATOM | 807 | CB | GLU | A | 469 | −7.649 | 14.613 | 5.759 | 1.00 | 23.02 | | A | C |
| ANISOU | 807 | CB | GLU | A | 469 | 2889 | 2932 | 2924 | −20 | −16 | −5 | A | C |
| ATOM | 810 | CG | GLU | A | 469 | −8.749 | 13.562 | 5.624 | 1.00 | 24.14 | | A | C |
| ANISOU | 810 | CG | GLU | A | 469 | 3042 | 3075 | 3054 | −76 | −36 | 51 | A | C |
| ATOM | 813 | CD | GLU | A | 469 | −8.315 | 12.246 | 4.988 | 1.00 | 27.42 | | A | C |
| ANISOU | 813 | CD | GLU | A | 469 | 3495 | 3409 | 3512 | 42 | 0 | −28 | A | C |
| ATOM | 814 | OE1 | GLU | A | 469 | −7.176 | 12.125 | 4.492 | 1.00 | 29.73 | | A | O |
| ANISOU | 814 | OE1 | GLU | A | 469 | 3673 | 3893 | 3729 | −41 | 77 | 19 | A | O |
| ATOM | 815 | OE2 | GLU | A | 469 | −9.131 | 11.292 | 4.989 | 1.00 | 29.95 | | A | O |
| ANISOU | 815 | OE2 | GLU | A | 469 | 3640 | 3810 | 3927 | −122 | −5 | −21 | A | O |
| ATOM | 816 | C | GLU | A | 469 | −6.367 | 13.346 | 7.498 | 1.00 | 23.42 | | A | C |
| ANISOU | 816 | C | GLU | A | 469 | 2990 | 2925 | 2984 | −23 | −15 | 5 | A | C |
| ATOM | 817 | O | GLU | A | 469 | −6.947 | 12.495 | 8.184 | 1.00 | 23.03 | | A | O |
| ANISOU | 817 | O | GLU | A | 469 | 2973 | 2852 | 2922 | −19 | −17 | 4 | A | O |
| ATOM | 818 | N | LYS | A | 470 | −5.152 | 13.163 | 7.002 | 1.00 | 24.57 | | A | N |
| ANISOU | 818 | N | LYS | A | 470 | 3089 | 3097 | 3150 | −20 | −4 | −14 | A | N |
| ATOM | 820 | CA | LYS | A | 470 | −4.380 | 11.953 | 7.273 | 1.00 | 25.96 | | A | C |
| ANISOU | 820 | CA | LYS | A | 470 | 3290 | 3239 | 3333 | −4 | −10 | 2 | A | C |
| ATOM | 822 | CB | LYS | A | 470 | −3.026 | 12.008 | 6.559 | 1.00 | 26.85 | | A | C |
| ANISOU | 822 | CB | LYS | A | 470 | 3374 | 3364 | 3463 | −2 | 21 | 0 | A | C |
| ATOM | 825 | CG | LYS | A | 470 | −3.074 | 12.031 | 5.046 | 1.00 | 28.91 | | A | C |
| ANISOU | 825 | CG | LYS | A | 470 | 3680 | 3666 | 3637 | −10 | −9 | −25 | A | C |
| ATOM | 828 | CD | LYS | A | 470 | −1.669 | 12.255 | 4.471 | 1.00 | 30.89 | | A | C |
| ANISOU | 828 | CD | LYS | A | 470 | 3826 | 3954 | 3958 | −5 | 35 | 17 | A | C |
| ATOM | 831 | CE | LYS | A | 470 | −1.675 | 12.400 | 2.954 | 1.00 | 32.45 | | A | C |
| ANISOU | 831 | CE | LYS | A | 470 | 4115 | 4158 | 4055 | −20 | 17 | 3 | A | C |
| ATOM | 834 | NZ | LYS | A | 470 | −1.310 | 13.782 | 2.473 | 1.00 | 33.67 | | A | N |
| ANISOU | 834 | NZ | LYS | A | 470 | 4283 | 4241 | 4268 | −30 | −19 | 17 | A | N |
| ATOM | 838 | C | LYS | A | 470 | −4.152 | 11.789 | 8.774 | 1.00 | 26.18 | | A | C |
| ANISOU | 838 | C | LYS | A | 470 | 3302 | 3289 | 3355 | −7 | 7 | −5 | A | C |
| ATOM | 839 | O | LYS | A | 470 | −4.404 | 10.731 | 9.339 | 1.00 | 27.01 | | A | O |
| ANISOU | 839 | O | LYS | A | 470 | 3425 | 3344 | 3493 | 32 | 7 | 35 | A | O |
| ATOM | 840 | N | PHE | A | 471 | −3.695 | 12.853 | 9.421 | 1.00 | 25.95 | | A | N |
| ANISOU | 840 | N | PHE | A | 471 | 3261 | 3255 | 3343 | −10 | 2 | −8 | A | N |
| ATOM | 842 | CA | PHE | A | 471 | −3.362 | 12.817 | 10.845 | 1.00 | 25.82 | | A | C |
| ANISOU | 842 | CA | PHE | A | 471 | 3257 | 3251 | 3302 | −17 | −1 | 22 | A | C |
| ATOM | 844 | CB | PHE | A | 471 | −2.694 | 14.122 | 11.277 | 1.00 | 26.48 | | A | C |
| ANISOU | 844 | CB | PHE | A | 471 | 3375 | 3316 | 3369 | −26 | −17 | 1 | A | C |
| ATOM | 847 | CG | PHE | A | 471 | −1.430 | 14.433 | 10.539 | 1.00 | 29.15 | | A | C |
| ANISOU | 847 | CG | PHE | A | 471 | 3612 | 3787 | 3677 | −3 | 37 | 21 | A | C |
| ATOM | 848 | CD1 | PHE | A | 471 | −0.745 | 13.453 | 9.816 | 1.00 | 31.05 | | A | C |
| ANISOU | 848 | CD1 | PHE | A | 471 | 3928 | 3921 | 3945 | 45 | 32 | −44 | A | C |
| ATOM | 850 | CE1 | PHE | A | 471 | 0.418 | 13.767 | 9.134 | 1.00 | 32.18 | | A | C |
| ANISOU | 850 | CE1 | PHE | A | 471 | 4022 | 4066 | 4138 | −23 | 51 | −12 | A | C |
| ATOM | 852 | CZ | PHE | A | 471 | 0.913 | 15.059 | 9.174 | 1.00 | 32.01 | | A | C |
| ANISOU | 852 | CZ | PHE | A | 471 | 4055 | 4034 | 4073 | 6 | 22 | 24 | A | C |
| ATOM | 854 | CE2 | PHE | A | 471 | 0.234 | 16.031 | 9.882 | 1.00 | 31.70 | | A | C |
| ANISOU | 854 | CE2 | PHE | A | 471 | 4012 | 4053 | 3979 | −58 | 29 | 14 | A | C |
| ATOM | 856 | CD2 | PHE | A | 471 | −0.930 | 15.722 | 10.554 | 1.00 | 30.89 | | A | C |
| ANISOU | 856 | CD2 | PHE | A | 471 | 3892 | 3928 | 3915 | −27 | 6 | −36 | A | C |
| ATOM | 858 | C | PHE | A | 471 | −4.561 | 12.604 | 11.736 | 1.00 | 25.07 | | A | C |
| ANISOU | 858 | C | PHE | A | 471 | 3156 | 3158 | 3210 | −6 | −21 | 6 | A | C |
| ATOM | 859 | O | PHE | A | 471 | −4.459 | 11.957 | 12.773 | 1.00 | 24.28 | | A | O |
| ANISOU | 859 | O | PHE | A | 471 | 3004 | 3006 | 3214 | −103 | −44 | 40 | A | O |
| ATOM | 860 | N | MET | A | 472 | −5.689 | 13.182 | 11.351 | 1.00 | 24.49 | | A | N |
| ANISOU | 860 | N | MET | A | 472 | 3096 | 3084 | 3123 | −25 | −17 | 23 | A | N |
| ATOM | 862 | CA | MET | A | 472 | −6.891 | 13.091 | 12.155 | 1.00 | 24.34 | | A | C |
| ANISOU | 862 | CA | MET | A | 472 | 3089 | 3066 | 3094 | −10 | 3 | 16 | A | C |
| ATOM | 864 | CB | MET | A | 472 | −7.927 | 14.118 | 11.710 | 1.00 | 24.75 | | A | C |
| ANISOU | 864 | CB | MET | A | 472 | 3163 | 3134 | 3106 | 15 | −8 | 24 | A | C |
| ATOM | 867 | CG | MET | A | 472 | −7.468 | 15.528 | 11.941 | 1.00 | 27.04 | | A | C |
| ANISOU | 867 | CG | MET | A | 472 | 3505 | 3408 | 3362 | −20 | −50 | −18 | A | C |
| ATOM | 870 | SD | MET | A | 472 | −7.230 | 15.809 | 13.673 | 1.00 | 30.11 | | A | S |
| ANISOU | 870 | SD | MET | A | 472 | 4137 | 3871 | 3430 | −73 | −82 | 28 | A | S |
| ATOM | 871 | CE | MET | A | 472 | −8.902 | 16.260 | 14.143 | 1.00 | 30.30 | | A | C |
| ANISOU | 871 | CE | MET | A | 472 | 3964 | 3785 | 3761 | −30 | −40 | −16 | A | C |
| ATOM | 875 | C | MET | A | 472 | −7.434 | 11.683 | 12.043 | 1.00 | 23.47 | | A | C |
| ANISOU | 875 | C | MET | A | 472 | 2948 | 2948 | 3018 | −5 | −2 | −10 | A | C |
| ATOM | 876 | O | MET | A | 472 | −7.835 | 11.105 | 13.046 | 1.00 | 22.54 | | A | O |
| ANISOU | 876 | O | MET | A | 472 | 2782 | 2692 | 3087 | −28 | 21 | −16 | A | O |
| ATOM | 877 | N | SER | A | 473 | −7.397 | 11.134 | 10.835 | 1.00 | 23.29 | | A | N |
| ANISOU | 877 | N | SER | A | 473 | 2910 | 2915 | 3022 | 10 | 5 | 10 | A | N |
| ATOM | 879 | CA | SER | A | 473 | −7.778 | 9.741 | 10.595 | 1.00 | 23.99 | | A | C |
| ANISOU | 879 | CA | SER | A | 473 | 3050 | 2995 | 3069 | −34 | 15 | −23 | A | C |
| ATOM | 881 | CB | SER | A | 473 | −7.530 | 9.365 | 9.131 | 1.00 | 24.56 | | A | C |
| ANISOU | 881 | CB | SER | A | 473 | 3154 | 3079 | 3098 | −30 | 4 | −34 | A | C |
| ATOM | 884 | OG | SER | A | 473 | −8.335 | 10.139 | 8.276 | 1.00 | 27.20 | | A | O |
| ANISOU | 884 | OG | SER | A | 473 | 3512 | 3346 | 3477 | 37 | 1 | −39 | A | O |
| ATOM | 886 | C | SER | A | 473 | −7.010 | 8.788 | 11.488 | 1.00 | 23.83 | | A | C |
| ANISOU | 886 | C | SER | A | 473 | 3037 | 2969 | 3047 | −42 | 38 | −29 | A | C |
| ATOM | 887 | O | SER | A | 473 | −7.588 | 7.883 | 12.086 | 1.00 | 23.99 | | A | O |
| ANISOU | 887 | O | SER | A | 473 | 3186 | 2840 | 3086 | −112 | 117 | −86 | A | O |
| ATOM | 888 | N | GLU | A | 474 | −5.702 | 8.990 | 11.573 | 1.00 | 23.71 | | A | N |
| ANISOU | 888 | N | GLU | A | 474 | 3023 | 2935 | 3047 | −33 | −2 | −6 | A | N |
| ATOM | 890 | CA | GLU | A | 474 | −4.829 | 8.150 | 12.382 | 1.00 | 23.37 | | A | C |
| ANISOU | 890 | CA | GLU | A | 474 | 2931 | 2935 | 3012 | −36 | 15 | 7 | A | C |
| ATOM | 892 | CB | GLU | A | 474 | −3.360 | 8.466 | 12.059 | 1.00 | 24.34 | | A | C |
| ANISOU | 892 | CB | GLU | A | 474 | 2989 | 3061 | 3198 | −30 | −1 | 4 | A | C |
| ATOM | 895 | CG | GLU | A | 474 | −2.941 | 8.037 | 10.651 | 1.00 | 27.51 | | A | C |
| ANISOU | 895 | CG | GLU | A | 474 | 3550 | 3482 | 3420 | 12 | −3 | −55 | A | C |
| ATOM | 898 | CD | GLU | A | 474 | −1.783 | 8.853 | 10.090 | 1.00 | 32.17 | | A | C |
| ANISOU | 898 | CD | GLU | A | 474 | 3968 | 4118 | 4136 | −73 | 80 | 27 | A | C |
| ATOM | 899 | OE1 | GLU | A | 474 | −1.100 | 9.553 | 10.871 | 1.00 | 34.14 | | A | O |
| ANISOU | 899 | OE1 | GLU | A | 474 | 4302 | 4334 | 4336 | −34 | −37 | −99 | A | O |
| ATOM | 900 | OE2 | GLU | A | 474 | −1.547 | 8.794 | 8.859 | 1.00 | 35.20 | | A | O |
| ANISOU | 900 | OE2 | GLU | A | 474 | 4516 | 4598 | 4258 | 0 | 27 | −19 | A | O |
| ATOM | 901 | C | GLU | A | 474 | −5.108 | 8.341 | 13.861 | 1.00 | 21.79 | | A | C |
| ANISOU | 901 | C | GLU | A | 474 | 2710 | 2682 | 2887 | −22 | 3 | −5 | A | C |
| ATOM | 902 | O | GLU | A | 474 | −5.080 | 7.382 | 14.633 | 1.00 | 20.36 | | A | O |
| ANISOU | 902 | O | GLU | A | 474 | 2363 | 2556 | 2814 | −137 | 72 | 75 | A | O |
| ATOM | 903 | N | ALA | A | 475 | −5.394 | 9.568 | 14.267 | 1.00 | 20.31 | | A | N |
| ANISOU | 903 | N | ALA | A | 475 | 2491 | 2500 | 2723 | −73 | 41 | 22 | A | N |
| ATOM | 905 | CA | ALA | A | 475 | −5.705 | 9.834 | 15.661 | 1.00 | 19.50 | | A | C |
| ANISOU | 905 | CA | ALA | A | 475 | 2479 | 2348 | 2581 | −50 | −14 | −20 | A | C |
| ATOM | 907 | CB | ALA | A | 475 | −5.826 | 11.348 | 15.893 | 1.00 | 19.18 | | A | C |
| ANISOU | 907 | CB | ALA | A | 475 | 2454 | 2292 | 2539 | −14 | −30 | −4 | A | C |
| ATOM | 911 | C | ALA | A | 475 | −6.986 | 9.113 | 16.131 | 1.00 | 19.35 | | A | C |
| ANISOU | 911 | C | ALA | A | 475 | 2492 | 2328 | 2531 | −47 | −3 | −25 | A | C |
| ATOM | 912 | O | ALA | A | 475 | −7.046 | 8.634 | 17.256 | 1.00 | 18.32 | | A | O |
| ANISOU | 912 | O | ALA | A | 475 | 2470 | 1974 | 2514 | −193 | −54 | −98 | A | O |
| ATOM | 913 | N | VAL | A | 476 | −7.999 | 9.054 | 15.271 | 1.00 | 19.06 | | A | N |
| ANISOU | 913 | N | VAL | A | 476 | 2442 | 2313 | 2486 | −61 | 49 | −62 | A | N |
| ATOM | 915 | CA | VAL | A | 476 | −9.257 | 8.358 | 15.587 | 1.00 | 19.09 | | A | C |
| ANISOU | 915 | CA | VAL | A | 476 | 2437 | 2331 | 2484 | −36 | 28 | −40 | A | C |
| ATOM | 917 | CB | VAL | A | 476 | −10.349 | 8.645 | 14.514 | 1.00 | 19.07 | | A | C |
| ANISOU | 917 | CB | VAL | A | 476 | 2419 | 2337 | 2487 | −16 | 25 | −39 | A | C |
| ATOM | 919 | CG1 | VAL | A | 476 | −11.592 | 7.797 | 14.731 | 1.00 | 20.08 | | A | C |
| ANISOU | 919 | CG1 | VAL | A | 476 | 2462 | 2561 | 2607 | −3 | 26 | −64 | A | C |
| ATOM | 923 | CG2 | VAL | A | 476 | −10.720 | 10.137 | 14.528 | 1.00 | 19.60 | | A | C |
| ANISOU | 923 | CG2 | VAL | A | 476 | 2453 | 2432 | 2561 | −14 | 2 | −40 | A | C |
| ATOM | 927 | C | VAL | A | 476 | −8.985 | 6.865 | 15.752 | 1.00 | 18.83 | | A | C |
| ANISOU | 927 | C | VAL | A | 476 | 2407 | 2308 | 2439 | −46 | 54 | −57 | A | C |
| ATOM | 928 | O | VAL | A | 476 | −9.568 | 6.231 | 16.613 | 1.00 | 18.22 | | A | O |
| ANISOU | 928 | O | VAL | A | 476 | 2315 | 2188 | 2420 | −172 | 161 | −183 | A | O |
| ATOM | 929 | N | ILE | A | 477 | −8.057 | 6.311 | 14.975 | 1.00 | 18.97 | | A | N |
| ANISOU | 929 | N | ILE | A | 477 | 2394 | 2359 | 2454 | −51 | 89 | −45 | A | N |
| ATOM | 931 | CA | ILE | A | 477 | −7.686 | 4.906 | 15.167 | 1.00 | 18.91 | | A | C |
| ANISOU | 931 | CA | ILE | A | 477 | 2373 | 2323 | 2488 | −46 | 76 | −38 | A | C |
| ATOM | 933 | CB | ILE | A | 477 | −6.741 | 4.397 | 14.062 | 1.00 | 19.56 | | A | C |
| ANISOU | 933 | CB | ILE | A | 477 | 2480 | 2428 | 2522 | −32 | 85 | −17 | A | C |
| ATOM | 935 | CG1 | ILE | A | 477 | −7.467 | 4.333 | 12.726 | 1.00 | 20.91 | | A | C |
| ANISOU | 935 | CG1 | ILE | A | 477 | 2585 | 2631 | 2728 | −18 | 17 | −29 | A | C |
| ATOM | 938 | CD1 | ILE | A | 477 | −6.537 | 4.159 | 11.530 | 1.00 | 22.97 | | A | C |
| ANISOU | 938 | CD1 | ILE | A | 477 | 2952 | 2899 | 2875 | −24 | 97 | −40 | A | C |
| ATOM | 942 | CG2 | ILE | A | 477 | −6.178 | 3.022 | 14.439 | 1.00 | 20.66 | | A | C |
| ANISOU | 942 | CG2 | ILE | A | 477 | 2590 | 2596 | 2661 | −19 | 102 | 6 | A | C |
| ATOM | 946 | C | ILE | A | 477 | −7.042 | 4.729 | 16.538 | 1.00 | 18.42 | | A | C |
| ANISOU | 946 | C | ILE | A | 477 | 2339 | 2226 | 2431 | −12 | 63 | −80 | A | C |
| ATOM | 947 | O | ILE | A | 477 | −7.390 | 3.842 | 17.310 | 1.00 | 17.83 | | A | O |
| ANISOU | 947 | O | ILE | A | 477 | 2273 | 2119 | 2382 | −161 | 157 | −199 | A | O |
| ATOM | 948 | N | MET | A | 478 | −6.117 | 5.617 | 16.866 | 1.00 | 18.34 | | A | N |
| ANISOU | 948 | N | MET | A | 478 | 2292 | 2195 | 2482 | −71 | 41 | 2 | A | N |
| ATOM | 950 | CA | MET | A | 478 | −5.446 | 5.557 | 18.143 | 1.00 | 19.38 | | A | C |
| ANISOU | 950 | CA | MET | A | 478 | 2492 | 2344 | 2527 | −62 | −2 | −24 | A | C |
| ATOM | 952 | CB | MET | A | 478 | −4.323 | 6.616 | 18.231 | 1.00 | 20.22 | | A | C |
| ANISOU | 952 | CB | MET | A | 478 | 2563 | 2437 | 2681 | −72 | −34 | −7 | A | C |
| ATOM | 955 | CG | MET | A | 478 | −3.109 | 6.353 | 17.341 | 1.00 | 22.57 | | A | C |
| ANISOU | 955 | CG | MET | A | 478 | 2917 | 2795 | 2863 | −86 | 75 | −23 | A | C |
| ATOM | 958 | SD | MET | A | 478 | −2.232 | 4.756 | 17.613 | 1.00 | 24.48 | | A | S |
| ANISOU | 958 | SD | MET | A | 478 | 3183 | 2881 | 3237 | −176 | 60 | 31 | A | S |
| ATOM | 959 | CE | MET | A | 478 | −1.864 | 4.806 | 19.305 | 1.00 | 23.71 | | A | C |
| ANISOU | 959 | CE | MET | A | 478 | 3066 | 2860 | 3080 | −32 | 0 | 45 | A | C |
| ATOM | 963 | C | MET | A | 478 | −6.413 | 5.699 | 19.301 | 1.00 | 18.90 | | A | C |
| ANISOU | 963 | C | MET | A | 478 | 2434 | 2267 | 2479 | −110 | −45 | −50 | A | C |
| ATOM | 964 | O | MET | A | 478 | −6.239 | 5.043 | 20.309 | 1.00 | 18.91 | | A | O |
| ANISOU | 964 | O | MET | A | 478 | 2513 | 2153 | 2518 | −197 | −100 | −77 | A | O |
| ATOM | 965 | N | LYS | A | 479 | −7.421 | 6.562 | 19.159 | 1.00 | 18.61 | | A | N |
| ANISOU | 965 | N | LYS | A | 479 | 2405 | 2246 | 2419 | −113 | −51 | −27 | A | N |
| ATOM | 967 | CA | LYS | A | 479 | −8.463 | 6.735 | 20.169 | 1.00 | 19.16 | | A | C |
| ANISOU | 967 | CA | LYS | A | 479 | 2498 | 2386 | 2393 | −81 | −7 | −48 | A | C |
| ATOM | 969 | CB | LYS | A | 479 | −9.507 | 7.770 | 19.717 | 1.00 | 20.24 | | A | C |
| ANISOU | 969 | CB | LYS | A | 479 | 2670 | 2500 | 2517 | −54 | −20 | −21 | A | C |
| ATOM | 972 | CG | LYS | A | 479 | −10.627 | 8.023 | 20.746 | 1.00 | 22.66 | | A | C |
| ANISOU | 972 | CG | LYS | A | 479 | 2887 | 2894 | 2826 | −53 | 68 | −67 | A | C |
| ATOM | 975 | CD | LYS | A | 479 | −11.477 | 9.232 | 20.413 | 1.00 | 26.49 | | A | C |
| ANISOU | 975 | CD | LYS | A | 479 | 3344 | 3355 | 3365 | 83 | 0 | 8 | A | C |
| ATOM | 978 | CE | LYS | A | 479 | −12.825 | 9.209 | 21.157 | 1.00 | 28.20 | | A | C |
| ANISOU | 978 | CE | LYS | A | 479 | 3530 | 3639 | 3546 | −21 | 78 | 2 | A | C |
| ATOM | 981 | NZ | LYS | A | 479 | −13.953 | 8.782 | 20.289 | 1.00 | 30.33 | | A | N |
| ANISOU | 981 | NZ | LYS | A | 479 | 3791 | 3947 | 3784 | −59 | −22 | −26 | A | N |
| ATOM | 985 | C | LYS | A | 479 | −9.159 | 5.407 | 20.529 | 1.00 | 18.39 | | A | C |
| ANISOU | 985 | C | LYS | A | 479 | 2426 | 2311 | 2249 | −99 | 4 | −23 | A | C |
| ATOM | 986 | O | LYS | A | 479 | −9.546 | 5.172 | 21.685 | 1.00 | 18.52 | | A | O |
| ANISOU | 986 | O | LYS | A | 479 | 2491 | 2351 | 2192 | −180 | −49 | −146 | A | O |
| ATOM | 987 | N | ASN | A | 480 | −9.356 | 4.564 | 19.523 | 1.00 | 16.96 | | A | N |
| ANISOU | 987 | N | ASN | A | 480 | 2237 | 2099 | 2106 | −34 | 18 | −14 | A | N |
| ATOM | 989 | CA | ASN | A | 480 | −10.026 | 3.286 | 19.703 | 1.00 | 16.98 | | A | C |
| ANISOU | 989 | CA | ASN | A | 480 | 2160 | 2145 | 2147 | −13 | −7 | −11 | A | C |
| ATOM | 991 | CB | ASN | A | 480 | −10.698 | 2.858 | 18.401 | 1.00 | 16.39 | | A | C |
| ANISOU | 991 | CB | ASN | A | 480 | 2076 | 2060 | 2090 | −37 | 4 | −52 | A | C |
| ATOM | 994 | CG | ASN | A | 480 | −11.934 | 3.639 | 18.112 | 1.00 | 15.29 | | A | C |
| ANISOU | 994 | CG | ASN | A | 480 | 1897 | 2026 | 1885 | −129 | 0 | −70 | A | C |
| ATOM | 995 | OD1 | ASN | A | 480 | −11.942 | 4.557 | 17.280 | 1.00 | 18.43 | | A | O |
| ANISOU | 995 | OD1 | ASN | A | 480 | 2446 | 2331 | 2223 | −208 | −96 | 46 | A | O |
| ATOM | 996 | ND2 | ASN | A | 480 | −13.011 | 3.303 | 18.816 | 1.00 | 13.78 | | A | N |
| ANISOU | 996 | ND2 | ASN | A | 480 | 1582 | 1892 | 1762 | −370 | −146 | −128 | A | N |
| ATOM | 999 | C | ASN | A | 480 | −9.129 | 2.159 | 20.204 | 1.00 | 17.53 | | A | C |
| ANISOU | 999 | C | ASN | A | 480 | 2223 | 2210 | 2226 | 19 | −15 | 8 | A | C |
| ATOM | 1000 | O | ASN | A | 480 | −9.622 | 1.111 | 20.641 | 1.00 | 18.79 | | A | O |
| ANISOU | 1000 | O | ASN | A | 480 | 2245 | 2368 | 2526 | −45 | 0 | −13 | A | O |
| ATOM | 1001 | N | LEU | A | 481 | −7.820 | 2.343 | 20.127 | 1.00 | 18.67 | | A | N |
| ANISOU | 1001 | N | LEU | A | 481 | 2339 | 2379 | 2372 | −11 | −18 | 36 | A | N |
| ATOM | 1003 | CA | LEU | A | 481 | −6.906 | 1.361 | 20.703 | 1.00 | 19.92 | | A | C |
| ANISOU | 1003 | CA | LEU | A | 481 | 2491 | 2490 | 2588 | 9 | 4 | 21 | A | C |
| ATOM | 1005 | CB | LEU | A | 481 | −5.514 | 1.472 | 20.095 | 1.00 | 19.88 | | A | C |
| ANISOU | 1005 | CB | LEU | A | 481 | 2462 | 2479 | 2611 | −37 | 7 | 31 | A | C |
| ATOM | 1008 | CG | LEU | A | 481 | −5.337 | 0.959 | 18.682 | 1.00 | 19.33 | | A | C |
| ANISOU | 1008 | CG | LEU | A | 481 | 2395 | 2422 | 2526 | −44 | 2 | 21 | A | C |
| ATOM | 1010 | CD1 | LEU | A | 481 | −3.960 | 1.337 | 18.187 | 1.00 | 19.58 | | A | C |
| ANISOU | 1010 | CD1 | LEU | A | 481 | 2355 | 2496 | 2587 | −44 | 42 | −36 | A | C |
| ATOM | 1014 | CD2 | LEU | A | 481 | −5.512 | −0.541 | 18.559 | 1.00 | 18.61 | | A | C |
| ANISOU | 1014 | CD2 | LEU | A | 481 | 2201 | 2342 | 2528 | −48 | −8 | −53 | A | C |
| ATOM | 1018 | C | LEU | A | 481 | −6.826 | 1.556 | 22.207 | 1.00 | 20.75 | | A | C |
| ANISOU | 1018 | C | LEU | A | 481 | 2607 | 2605 | 2671 | −7 | −31 | 19 | A | C |
| ATOM | 1019 | O | LEU | A | 481 | −6.351 | 2.579 | 22.698 | 1.00 | 23.44 | | A | O |
| ANISOU | 1019 | O | LEU | A | 481 | 3130 | 2817 | 2960 | −75 | −85 | 30 | A | O |
| ATOM | 1020 | N | ASP | A | 482 | −7.252 | 0.544 | 22.936 | 1.00 | 19.94 | | A | N |
| ANISOU | 1020 | N | ASP | A | 482 | 2354 | 2547 | 2673 | −12 | 4 | 43 | A | N |
| ATOM | 1022 | CA | ASP | A | 482 | −7.259 | 0.567 | 24.385 | 1.00 | 20.20 | | A | C |
| ANISOU | 1022 | CA | ASP | A | 482 | 2423 | 2593 | 2658 | −6 | −14 | −1 | A | C |
| ATOM | 1024 | CB | ASP | A | 482 | −8.699 | 0.585 | 24.928 | 1.00 | 21.21 | | A | C |
| ANISOU | 1024 | CB | ASP | A | 482 | 2492 | 2737 | 2829 | −7 | 28 | 36 | A | C |
| ATOM | 1027 | CG | ASP | A | 482 | −8.795 | 1.086 | 26.367 | 1.00 | 24.55 | | A | C |
| ANISOU | 1027 | CG | ASP | A | 482 | 3026 | 3244 | 3055 | −4 | 29 | −54 | A | C |
| ATOM | 1028 | OD1 | ASP | A | 482 | −7.776 | 1.153 | 27.090 | 1.00 | 26.94 | | A | O |
| ANISOU | 1028 | OD1 | ASP | A | 482 | 3169 | 3589 | 3477 | 68 | 42 | −84 | A | O |
| ATOM | 1029 | OD2 | ASP | A | 482 | −9.894 | 1.432 | 26.884 | 1.00 | 29.24 | | A | O |
| ANISOU | 1029 | OD2 | ASP | A | 482 | 3376 | 3929 | 3802 | 104 | 152 | −37 | A | O |
| ATOM | 1030 | C | ASP | A | 482 | −6.532 | −0.698 | 24.788 | 1.00 | 19.47 | | A | C |
| ANISOU | 1030 | C | ASP | A | 482 | 2366 | 2458 | 2573 | −81 | −51 | 37 | A | C |
| ATOM | 1031 | O | ASP | A | 482 | −7.070 | −1.795 | 24.722 | 1.00 | 21.32 | | A | O |
| ANISOU | 1031 | O | ASP | A | 482 | 2652 | 2592 | 2854 | −109 | −142 | 65 | A | O |
| ATOM | 1032 | N | HIS | A | 483 | −5.265 | −0.533 | 25.136 | 1.00 | 16.58 | | A | N |
| ANISOU | 1032 | N | HIS | A | 483 | 2011 | 2099 | 2188 | −22 | −11 | −23 | A | N |
| ATOM | 1034 | CA | HIS | A | 483 | −4.430 | −1.613 | 25.569 | 1.00 | 15.45 | | A | C |
| ANISOU | 1034 | CA | HIS | A | 483 | 1977 | 1944 | 1946 | −48 | 1 | 0 | A | C |
| ATOM | 1036 | CB | HIS | A | 483 | −3.614 | −2.176 | 24.393 | 1.00 | 15.27 | | A | C |
| ANISOU | 1036 | CB | HIS | A | 483 | 1873 | 1981 | 1945 | −27 | 9 | −68 | A | C |
| ATOM | 1039 | CG | HIS | A | 483 | −2.972 | −3.478 | 24.705 | 1.00 | 15.11 | | A | C |
| ANISOU | 1039 | CG | HIS | A | 483 | 2091 | 1955 | 1696 | −45 | −4 | −1 | A | C |
| ATOM | 1040 | ND1 | HIS | A | 483 | −1.888 | −3.587 | 25.541 | 1.00 | 14.86 | | A | N |
| ANISOU | 1040 | ND1 | HIS | A | 483 | 1922 | 1862 | 1862 | −43 | 20 | −94 | A | N |
| ATOM | 1042 | CE1 | HIS | A | 483 | −1.587 | −4.860 | 25.707 | 1.00 | 16.29 | | A | C |
| ANISOU | 1042 | CE1 | HIS | A | 483 | 2204 | 1904 | 2080 | 116 | 12 | −177 | A | C |
| ATOM | 1044 | NE2 | HIS | A | 483 | −2.413 | −5.584 | 24.975 | 1.00 | 16.13 | | A | N |
| ANISOU | 1044 | NE2 | HIS | A | 483 | 2252 | 2054 | 1821 | −78 | −44 | 89 | A | N |
| ATOM | 1046 | CD2 | HIS | A | 483 | −3.281 | −4.744 | 24.323 | 1.00 | 16.71 | | A | C |
| ANISOU | 1046 | CD2 | HIS | A | 483 | 2200 | 1975 | 2174 | 40 | −78 | −81 | A | C |
| ATOM | 1048 | C | HIS | A | 483 | −3.501 | −1.061 | 26.647 | 1.00 | 14.08 | | A | C |
| ANISOU | 1048 | C | HIS | A | 483 | 1731 | 1775 | 1845 | 0 | 23 | −4 | A | C |
| ATOM | 1049 | O | HIS | A | 483 | −3.031 | 0.075 | 26.517 | 1.00 | 13.86 | | A | O |
| ANISOU | 1049 | O | HIS | A | 483 | 1538 | 1815 | 1913 | −9 | 136 | −45 | A | O |
| ATOM | 1050 | N | PRO | A | 484 | −3.250 | −1.822 | 27.717 | 1.00 | 13.40 | | A | N |
| ANISOU | 1050 | N | PRO | A | 484 | 1676 | 1608 | 1806 | −6 | 56 | −36 | A | N |
| ATOM | 1051 | CA | PRO | A | 484 | −2.360 | −1.331 | 28.765 | 1.00 | 13.42 | | A | C |
| ANISOU | 1051 | CA | PRO | A | 484 | 1685 | 1656 | 1757 | 68 | 41 | 0 | A | C |
| ATOM | 1053 | CB | PRO | A | 484 | −2.332 | −2.483 | 29.788 | 1.00 | 14.58 | | A | C |
| ANISOU | 1053 | CB | PRO | A | 484 | 1899 | 1792 | 1849 | −11 | 77 | 30 | A | C |
| ATOM | 1056 | CG | PRO | A | 484 | −3.446 | −3.345 | 29.452 | 1.00 | 15.97 | | A | C |
| ANISOU | 1056 | CG | PRO | A | 484 | 2026 | 1962 | 2077 | −23 | −86 | 4 | A | C |
| ATOM | 1059 | CD | PRO | A | 484 | −3.849 | −3.127 | 28.069 | 1.00 | 13.57 | | A | C |
| ANISOU | 1059 | CD | PRO | A | 484 | 1721 | 1667 | 1766 | −21 | 84 | −36 | A | C |
| ATOM | 1062 | C | PRO | A | 484 | −0.961 | −0.972 | 28.281 | 1.00 | 12.89 | | A | C |
| ANISOU | 1062 | C | PRO | A | 484 | 1678 | 1551 | 1668 | 15 | 21 | 1 | A | C |
| ATOM | 1063 | O | PRO | A | 484 | −0.298 | −0.240 | 28.987 | 1.00 | 12.93 | | A | O |
| ANISOU | 1063 | O | PRO | A | 484 | 1688 | 1539 | 1684 | 29 | −31 | −89 | A | O |
| ATOM | 1064 | N | HIS | A | 485 | −0.504 | −1.486 | 27.135 | 1.00 | 12.02 | | A | N |
| ANISOU | 1064 | N | HIS | A | 485 | 1536 | 1444 | 1587 | 14 | 25 | 16 | A | N |
| ATOM | 1066 | CA | HIS | A | 485 | 0.846 | −1.194 | 26.667 | 1.00 | 11.59 | | A | C |
| ANISOU | 1066 | CA | HIS | A | 485 | 1455 | 1436 | 1513 | 68 | 76 | 6 | A | C |
| ATOM | 1068 | CB | HIS | A | 485 | 1.704 | −2.445 | 26.786 | 1.00 | 12.42 | | A | C |
| ANISOU | 1068 | CB | HIS | A | 485 | 1506 | 1595 | 1618 | 36 | 40 | −21 | A | C |
| ATOM | 1071 | CG | HIS | A | 485 | 1.737 | −2.953 | 28.196 | 1.00 | 16.90 | | A | C |
| ANISOU | 1071 | CG | HIS | A | 485 | 2175 | 2294 | 1952 | 290 | 168 | 192 | A | C |
| ATOM | 1072 | ND1 | HIS | A | 485 | 2.884 | −3.185 | 28.896 | 1.00 | 24.89 | | A | N |
| ANISOU | 1072 | ND1 | HIS | A | 485 | 3030 | 3698 | 2726 | −296 | −189 | 291 | A | N |
| ATOM | 1074 | CE1 | HIS | A | 485 | 2.579 | −3.596 | 30.118 | 1.00 | 23.33 | | A | C |
| ANISOU | 1074 | CE1 | HIS | A | 485 | 3028 | 3348 | 2487 | −30 | 7 | 120 | A | C |
| ATOM | 1076 | NE2 | HIS | A | 485 | 1.285 | −3.609 | 30.242 | 1.00 | 20.63 | | A | N |
| ANISOU | 1076 | NE2 | HIS | A | 485 | 2810 | 2815 | 2211 | 35 | −75 | 218 | A | N |
| ATOM | 1078 | CD2 | HIS | A | 485 | 0.730 | −3.153 | 29.076 | 1.00 | 23.26 | | A | C |
| ANISOU | 1078 | CD2 | HIS | A | 485 | 2913 | 3335 | 2589 | −224 | 19 | 322 | A | C |
| ATOM | 1080 | C | HIS | A | 485 | 0.856 | −0.583 | 25.281 | 1.00 | 10.76 | | A | C |
| ANISOU | 1080 | C | HIS | A | 485 | 1357 | 1307 | 1423 | 42 | −20 | −15 | A | C |
| ATOM | 1081 | O | HIS | A | 485 | 1.791 | −0.771 | 24.511 | 1.00 | 10.12 | | A | O |
| ANISOU | 1081 | O | HIS | A | 485 | 1239 | 1166 | 1439 | 70 | −24 | −33 | A | O |
| ATOM | 1082 | N | ILE | A | 486 | −0.198 | 0.176 | 24.993 | 1.00 | 9.75 | | A | N |
| ANISOU | 1082 | N | ILE | A | 486 | 1225 | 1255 | 1224 | 101 | 32 | −30 | A | N |
| ATOM | 1084 | CA | ILE | A | 486 | −0.232 | 1.097 | 23.843 | 1.00 | 10.19 | | A | C |
| ANISOU | 1084 | CA | ILE | A | 486 | 1387 | 1160 | 1322 | 41 | −28 | −33 | A | C |
| ATOM | 1086 | CB | ILE | A | 486 | −1.268 | 0.655 | 22.782 | 1.00 | 9.74 | | A | C |
| ANISOU | 1086 | CB | ILE | A | 486 | 1305 | 1111 | 1284 | 74 | −35 | −40 | A | C |
| ATOM | 1088 | CG1 | ILE | A | 486 | −0.894 | −0.735 | 22.230 | 1.00 | 11.70 | | A | C |
| ANISOU | 1088 | CG1 | ILE | A | 486 | 1556 | 1206 | 1680 | 62 | −40 | 6 | A | C |
| ATOM | 1091 | CD1 | ILE | A | 486 | −1.888 | −1.277 | 21.230 | 1.00 | 12.79 | | A | C |
| ANISOU | 1091 | CD1 | ILE | A | 486 | 1576 | 1542 | 1738 | −5 | −71 | −11 | A | C |
| ATOM | 1095 | CG2 | ILE | A | 486 | −1.356 | 1.695 | 21.677 | 1.00 | 11.01 | | A | C |
| ANISOU | 1095 | CG2 | ILE | A | 486 | 1373 | 1557 | 1253 | 35 | 44 | 0 | A | C |
| ATOM | 1099 | C | ILE | A | 486 | −0.575 | 2.503 | 24.346 | 1.00 | 9.96 | | A | C |
| ANISOU | 1099 | C | ILE | A | 486 | 1290 | 1200 | 1291 | 72 | −2 | −51 | A | C |
| ATOM | 1100 | O | ILE | A | 486 | −1.434 | 2.673 | 25.223 | 1.00 | 10.61 | | A | O |
| ANISOU | 1100 | O | ILE | A | 486 | 1394 | 1177 | 1461 | 137 | 68 | −112 | A | O |
| ATOM | 1101 | N | VAL | A | 487 | 0.123 | 3.496 | 23.828 | 1.00 | 10.88 | | A | N |
| ANISOU | 1101 | N | VAL | A | 487 | 1433 | 1284 | 1415 | 68 | −32 | −33 | A | N |
| ATOM | 1103 | CA | VAL | A | 487 | −0.119 | 4.870 | 24.267 | 1.00 | 11.41 | | A | C |
| ANISOU | 1103 | CA | VAL | A | 487 | 1449 | 1356 | 1527 | 39 | 22 | −31 | A | C |
| ATOM | 1105 | CB | VAL | A | 487 | 0.729 | 5.886 | 23.464 | 1.00 | 11.90 | | A | C |
| ANISOU | 1105 | CB | VAL | A | 487 | 1523 | 1349 | 1647 | 40 | 23 | −31 | A | C |
| ATOM | 1107 | CG1 | VAL | A | 487 | 2.203 | 5.669 | 23.775 | 1.00 | 10.94 | | A | C |
| ANISOU | 1107 | CG1 | VAL | A | 487 | 1527 | 1338 | 1290 | −75 | 6 | −92 | A | C |
| ATOM | 1111 | CG2 | VAL | A | 487 | 0.431 | 5.805 | 21.981 | 1.00 | 14.05 | | A | C |
| ANISOU | 1111 | CG2 | VAL | A | 487 | 1875 | 1617 | 1845 | 82 | 40 | 35 | A | C |
| ATOM | 1115 | C | VAL | A | 487 | −1.613 | 5.215 | 24.177 | 1.00 | 12.14 | | A | C |
| ANISOU | 1115 | C | VAL | A | 487 | 1487 | 1487 | 1637 | 25 | 13 | −34 | A | C |
| ATOM | 1116 | O | VAL | A | 487 | −2.320 | 4.793 | 23.253 | 1.00 | 12.52 | | A | O |
| ANISOU | 1116 | O | VAL | A | 487 | 1452 | 1584 | 1720 | 174 | −17 | −47 | A | O |
| ATOM | 1117 | N | LYS | A | 488 | −2.072 | 5.943 | 25.185 | 1.00 | 12.49 | | A | N |
| ANISOU | 1117 | N | LYS | A | 488 | 1582 | 1418 | 1746 | −26 | 65 | −46 | A | N |
| ATOM | 1119 | CA | LYS | A | 488 | −3.478 | 6.269 | 25.322 | 1.00 | 13.72 | | A | C |
| ANISOU | 1119 | CA | LYS | A | 488 | 1732 | 1573 | 1909 | 79 | −44 | 1 | A | C |
| ATOM | 1121 | CB | LYS | A | 488 | −3.880 | 6.171 | 26.785 | 1.00 | 15.01 | | A | C |
| ANISOU | 1121 | CB | LYS | A | 488 | 1939 | 1768 | 1996 | 75 | 24 | 33 | A | C |
| ATOM | 1124 | CG | LYS | A | 488 | −5.340 | 6.581 | 27.022 | 1.00 | 18.79 | | A | C |
| ANISOU | 1124 | CG | LYS | A | 488 | 2195 | 2347 | 2597 | 114 | 4 | −67 | A | C |
| ATOM | 1127 | CD | LYS | A | 488 | −5.926 | 6.040 | 28.301 | 1.00 | 23.36 | | A | C |
| ANISOU | 1127 | CD | LYS | A | 488 | 2996 | 2977 | 2899 | 21 | 56 | 52 | A | C |
| ATOM | 1130 | CE | LYS | A | 488 | −7.455 | 5.893 | 28.165 | 1.00 | 25.89 | | A | C |
| ANISOU | 1130 | CE | LYS | A | 488 | 3144 | 3344 | 3348 | −9 | −37 | 28 | A | C |
| ATOM | 1133 | NZ | LYS | A | 488 | −8.211 | 5.808 | 29.464 | 1.00 | 28.78 | | A | N |
| ANISOU | 1133 | NZ | LYS | A | 488 | 3710 | 3660 | 3562 | −17 | 28 | 36 | A | N |
| ATOM | 1137 | C | LYS | A | 488 | −3.791 | 7.673 | 24.817 | 1.00 | 13.81 | | A | C |
| ANISOU | 1137 | C | LYS | A | 488 | 1813 | 1503 | 1930 | 7 | −9 | 14 | A | C |
| ATOM | 1138 | O | LYS | A | 488 | −3.234 | 8.669 | 25.327 | 1.00 | 13.74 | | A | O |
| ANISOU | 1138 | O | LYS | A | 488 | 1789 | 1333 | 2096 | 139 | −142 | 39 | A | O |
| ATOM | 1139 | N | LEU | A | 489 | −4.670 | 7.723 | 23.826 | 1.00 | 14.99 | | A | N |
| ANISOU | 1139 | N | LEU | A | 489 | 1997 | 1595 | 2104 | 30 | −56 | 15 | A | N |
| ATOM | 1141 | CA | LEU | A | 489 | −5.218 | 8.979 | 23.296 | 1.00 | 16.61 | | A | C |
| ANISOU | 1141 | CA | LEU | A | 489 | 2161 | 1908 | 2241 | 28 | −64 | 42 | A | C |
| ATOM | 1143 | CB | LEU | A | 489 | −5.445 | 8.889 | 21.783 | 1.00 | 17.93 | | A | C |
| ANISOU | 1143 | CB | LEU | A | 489 | 2381 | 2080 | 2351 | 27 | −59 | 0 | A | C |
| ATOM | 1146 | CG | LEU | A | 489 | −5.759 | 10.211 | 21.078 | 1.00 | 20.73 | | A | C |
| ANISOU | 1146 | CG | LEU | A | 489 | 2798 | 2384 | 2693 | −42 | −23 | 37 | A | C |
| ATOM | 1148 | CD1 | LEU | A | 489 | −5.312 | 10.130 | 19.618 | 1.00 | 22.63 | | A | C |
| ANISOU | 1148 | CD1 | LEU | A | 489 | 3054 | 2730 | 2815 | −2 | −45 | 33 | A | C |
| ATOM | 1152 | CD2 | LEU | A | 489 | −7.204 | 10.543 | 21.212 | 1.00 | 22.83 | | A | C |
| ANISOU | 1152 | CD2 | LEU | A | 489 | 2960 | 2703 | 3009 | 26 | −126 | −37 | A | C |
| ATOM | 1156 | C | LEU | A | 489 | −6.493 | 9.234 | 24.039 | 1.00 | 17.27 | | A | C |
| ANISOU | 1156 | C | LEU | A | 489 | 2161 | 2012 | 2386 | 0 | −56 | 53 | A | C |
| ATOM | 1157 | O | LEU | A | 489 | −7.442 | 8.439 | 23.953 | 1.00 | 19.13 | | A | O |
| ANISOU | 1157 | O | LEU | A | 489 | 2297 | 2041 | 2930 | −29 | −52 | 68 | A | O |
| ATOM | 1158 | N | ILE | A | 490 | −6.531 | 10.339 | 24.781 | 1.00 | 16.86 | | A | N |
| ANISOU | 1158 | N | ILE | A | 490 | 2071 | 2069 | 2263 | 27 | 24 | 26 | A | N |
| ATOM | 1160 | CA | ILE | A | 490 | −7.653 | 10.645 | 25.665 | 1.00 | 17.31 | | A | C |
| ANISOU | 1160 | CA | ILE | A | 490 | 2134 | 2134 | 2309 | 45 | 31 | 79 | A | C |
| ATOM | 1162 | CB | ILE | A | 490 | −7.153 | 11.481 | 26.830 | 1.00 | 17.50 | | A | C |
| ANISOU | 1162 | CB | ILE | A | 490 | 2191 | 2209 | 2249 | 42 | 30 | 61 | A | C |
| ATOM | 1164 | CG1 | ILE | A | 490 | −6.191 | 10.662 | 27.693 | 1.00 | 18.79 | | A | C |
| ANISOU | 1164 | CG1 | ILE | A | 490 | 2337 | 2365 | 2434 | 67 | 5 | 0 | A | C |
| ATOM | 1167 | CD1 | ILE | A | 490 | −5.432 | 11.484 | 28.687 | 1.00 | 19.29 | | A | C |
| ANISOU | 1167 | CD1 | ILE | A | 490 | 2445 | 2407 | 2475 | 17 | −16 | −56 | A | C |
| ATOM | 1171 | CG2 | ILE | A | 490 | −8.330 | 12.031 | 27.654 | 1.00 | 18.76 | | A | C |
| ANISOU | 1171 | CG2 | ILE | A | 490 | 2345 | 2390 | 2393 | 60 | 85 | 70 | A | C |
| ATOM | 1175 | C | ILE | A | 490 | −8.776 | 11.372 | 24.940 | 1.00 | 17.33 | | A | C |
| ANISOU | 1175 | C | ILE | A | 490 | 2102 | 2220 | 2260 | 3 | 13 | 97 | A | C |
| ATOM | 1176 | O | ILE | A | 490 | −9.951 | 11.089 | 25.159 | 1.00 | 18.68 | | A | O |
| ANISOU | 1176 | O | ILE | A | 490 | 2110 | 2361 | 2626 | −4 | −61 | 139 | A | O |
| ATOM | 1177 | N | GLY | A | 491 | −8.427 | 12.324 | 24.091 | 1.00 | 16.24 | | A | N |
| ANISOU | 1177 | N | GLY | A | 491 | 1928 | 2002 | 2238 | 21 | −3 | 88 | A | N |
| ATOM | 1179 | CA | GLY | A | 491 | −9.453 | 13.017 | 23.337 | 1.00 | 16.22 | | A | C |
| ANISOU | 1179 | CA | GLY | A | 491 | 1991 | 2001 | 2171 | 19 | −27 | 25 | A | C |
| ATOM | 1182 | C | GLY | A | 491 | −8.931 | 13.872 | 22.220 | 1.00 | 15.56 | | A | C |
| ANISOU | 1182 | C | GLY | A | 491 | 1888 | 1933 | 2092 | −8 | −28 | 20 | A | C |
| ATOM | 1183 | O | GLY | A | 491 | −7.740 | 14.041 | 22.046 | 1.00 | 14.32 | | A | O |
| ANISOU | 1183 | O | GLY | A | 491 | 1639 | 1560 | 2241 | −21 | −88 | 10 | A | O |
| ATOM | 1184 | N | ILE | A | 492 | −9.855 | 14.381 | 21.419 | 1.00 | 16.14 | | A | N |
| ANISOU | 1184 | N | ILE | A | 492 | 1926 | 1975 | 2229 | 19 | −48 | 39 | A | N |
| ATOM | 1186 | CA | ILE | A | 492 | −9.532 | 15.195 | 20.260 | 1.00 | 17.06 | | A | C |
| ANISOU | 1186 | CA | ILE | A | 492 | 2088 | 2137 | 2255 | 3 | −25 | 21 | A | C |
| ATOM | 1188 | CB | ILE | A | 492 | −9.742 | 14.397 | 18.950 | 1.00 | 17.37 | | A | C |
| ANISOU | 1188 | CB | ILE | A | 492 | 2124 | 2210 | 2264 | 6 | −30 | 12 | A | C |
| ATOM | 1190 | CG1 | ILE | A | 492 | −8.852 | 13.150 | 18.902 | 1.00 | 18.95 | | A | C |
| ANISOU | 1190 | CG1 | ILE | A | 492 | 2443 | 2336 | 2420 | 57 | 4 | −46 | A | C |
| ATOM | 1193 | CD1 | ILE | A | 492 | −9.244 | 12.169 | 17.797 | 1.00 | 20.06 | | A | C |
| ANISOU | 1193 | CD1 | ILE | A | 492 | 2623 | 2503 | 2495 | −61 | −65 | 6 | A | C |
| ATOM | 1197 | CG2 | ILE | A | 492 | −9.469 | 15.274 | 17.749 | 1.00 | 19.11 | | A | C |
| ANISOU | 1197 | CG2 | ILE | A | 492 | 2483 | 2378 | 2399 | −31 | −24 | 42 | A | C |
| ATOM | 1201 | C | ILE | A | 492 | −10.463 | 16.415 | 20.310 | 1.00 | 17.29 | | A | C |
| ANISOU | 1201 | C | ILE | A | 492 | 2054 | 2160 | 2356 | 26 | −25 | 52 | A | C |
| ATOM | 1202 | O | ILE | A | 492 | −11.686 | 16.280 | 20.428 | 1.00 | 17.49 | | A | O |
| ANISOU | 1202 | O | ILE | A | 492 | 2030 | 2126 | 2486 | −106 | −25 | 125 | A | O |
| ATOM | 1203 | N | ILE | A | 493 | −9.882 | 17.607 | 20.268 | 1.00 | 16.77 | | A | N |
| ANISOU | 1203 | N | ILE | A | 493 | 2017 | 2032 | 2322 | 89 | −22 | 63 | A | N |
| ATOM | 1205 | CA | ILE | A | 493 | −10.655 | 18.827 | 20.046 | 1.00 | 17.71 | | A | C |
| ANISOU | 1205 | CA | ILE | A | 493 | 2216 | 2141 | 2370 | 80 | −43 | 47 | A | C |
| ATOM | 1207 | CB | ILE | A | 493 | −10.193 | 19.940 | 20.958 | 1.00 | 17.74 | | A | C |
| ANISOU | 1207 | CB | ILE | A | 493 | 2203 | 2198 | 2339 | 118 | −25 | 46 | A | C |
| ATOM | 1209 | CG1 | ILE | A | 493 | −10.306 | 19.495 | 22.415 | 1.00 | 19.40 | | A | C |
| ANISOU | 1209 | CG1 | ILE | A | 493 | 2449 | 2457 | 2464 | 75 | −11 | 52 | A | C |
| ATOM | 1212 | CD1 | ILE | A | 493 | −9.703 | 20.456 | 23.344 | 1.00 | 21.40 | | A | C |
| ANISOU | 1212 | CD1 | ILE | A | 493 | 2677 | 2726 | 2726 | −3 | −36 | −32 | A | C |
| ATOM | 1216 | CG2 | ILE | A | 493 | −11.022 | 21.222 | 20.690 | 1.00 | 17.23 | | A | C |
| ANISOU | 1216 | CG2 | ILE | A | 493 | 2206 | 2039 | 2300 | 95 | −5 | 63 | A | C |
| ATOM | 1220 | C | ILE | A | 493 | −10.395 | 19.163 | 18.603 | 1.00 | 18.30 | | A | C |
| ANISOU | 1220 | C | ILE | A | 493 | 2336 | 2214 | 2402 | 80 | −27 | 31 | A | C |
| ATOM | 1221 | O | ILE | A | 493 | −9.308 | 19.592 | 18.238 | 1.00 | 17.19 | | A | O |
| ANISOU | 1221 | O | ILE | A | 493 | 2246 | 1979 | 2305 | 160 | −159 | 3 | A | O |
| ATOM | 1222 | N | GLU | A | 494 | −11.418 | 18.985 | 17.779 | 1.00 | 20.26 | | A | N |
| ANISOU | 1222 | N | GLU | A | 494 | 2566 | 2512 | 2619 | 10 | −82 | −4 | A | N |
| ATOM | 1224 | CA | GLU | A | 494 | −11.262 | 19.089 | 16.330 | 1.00 | 22.00 | | A | C |
| ANISOU | 1224 | CA | GLU | A | 494 | 2798 | 2788 | 2770 | 23 | −21 | 20 | A | C |
| ATOM | 1226 | CB | GLU | A | 494 | −12.443 | 18.395 | 15.635 | 1.00 | 22.98 | | A | C |
| ANISOU | 1226 | CB | GLU | A | 494 | 2893 | 2940 | 2896 | −10 | −54 | −33 | A | C |
| ATOM | 1229 | CG | GLU | A | 494 | −12.641 | 16.927 | 16.006 | 1.00 | 25.89 | | A | C |
| ANISOU | 1229 | CG | GLU | A | 494 | 3352 | 3198 | 3284 | 14 | −5 | 62 | A | C |
| ATOM | 1232 | CD | GLU | A | 494 | −13.947 | 16.341 | 15.472 | 1.00 | 30.25 | | A | C |
| ANISOU | 1232 | CD | GLU | A | 494 | 3768 | 3834 | 3891 | −107 | −67 | −25 | A | C |
| ATOM | 1233 | OE1 | GLU | A | 494 | −14.323 | 15.221 | 15.903 | 1.00 | 32.64 | | A | O |
| ANISOU | 1233 | OE1 | GLU | A | 494 | 4256 | 3953 | 4191 | −107 | −14 | 33 | A | O |
| ATOM | 1234 | OE2 | GLU | A | 494 | −14.608 | 16.997 | 14.628 | 1.00 | 32.77 | | A | O |
| ANISOU | 1234 | OE2 | GLU | A | 494 | 4137 | 4180 | 4133 | −15 | −110 | 61 | A | O |
| ATOM | 1235 | C | GLU | A | 494 | −11.165 | 20.550 | 15.875 | 1.00 | 22.26 | | A | C |
| ANISOU | 1235 | C | GLU | A | 494 | 2829 | 2822 | 2804 | 5 | −17 | 12 | A | C |
| ATOM | 1236 | O | GLU | A | 494 | −10.408 | 20.881 | 14.955 | 1.00 | 22.49 | | A | O |
| ANISOU | 1236 | O | GLU | A | 494 | 2881 | 2892 | 2771 | −2 | −34 | 46 | A | O |
| ATOM | 1237 | N | GLU | A | 495 | −11.892 | 21.421 | 16.566 | 1.00 | 22.55 | | A | N |
| ANISOU | 1237 | N | GLU | A | 495 | 2859 | 2872 | 2834 | 32 | −48 | 20 | A | N |
| ATOM | 1239 | CA | GLU | A | 495 | −12.028 | 22.821 | 16.164 | 1.00 | 23.10 | | A | C |
| ANISOU | 1239 | CA | GLU | A | 495 | 2913 | 2910 | 2953 | 8 | −26 | −1 | A | C |
| ATOM | 1241 | CB | GLU | A | 495 | −13.047 | 23.513 | 17.070 | 1.00 | 23.74 | | A | C |
| ANISOU | 1241 | CB | GLU | A | 495 | 3047 | 2983 | 2991 | 41 | −17 | −6 | A | C |
| ATOM | 1244 | CG | GLU | A | 495 | −14.487 | 23.077 | 16.818 | 1.00 | 25.77 | | A | C |
| ANISOU | 1244 | CG | GLU | A | 495 | 3199 | 3311 | 3280 | −10 | −24 | −23 | A | C |
| ATOM | 1247 | CD | GLU | A | 495 | −14.984 | 21.935 | 17.706 | 1.00 | 28.15 | | A | C |
| ANISOU | 1247 | CD | GLU | A | 495 | 3598 | 3530 | 3567 | −3 | 8 | 87 | A | C |
| ATOM | 1248 | OE1 | GLU | A | 495 | −14.178 | 21.250 | 18.372 | 1.00 | 28.67 | | A | O |
| ANISOU | 1248 | OE1 | GLU | A | 495 | 3561 | 3664 | 3666 | 86 | −2 | 42 | A | O |
| ATOM | 1249 | OE2 | GLU | A | 495 | −16.216 | 21.716 | 17.746 | 1.00 | 30.21 | | A | O |
| ANISOU | 1249 | OE2 | GLU | A | 495 | 3742 | 3840 | 3896 | −40 | −35 | 22 | A | O |
| ATOM | 1250 | C | GLU | A | 495 | −10.688 | 23.549 | 16.206 | 1.00 | 22.54 | | A | C |
| ANISOU | 1250 | C | GLU | A | 495 | 2852 | 2842 | 2869 | 26 | −20 | −60 | A | C |
| ATOM | 1251 | O | GLU | A | 495 | −9.768 | 23.126 | 16.933 | 1.00 | 21.30 | | A | O |
| ANISOU | 1251 | O | GLU | A | 495 | 2746 | 2563 | 2784 | −7 | −38 | 27 | A | O |
| ATOM | 1252 | N | GLU | A | 496 | −10.998 | 25.197 | 16.115 | 0.00 | 27.68 | | A | N |
| ANISOU | 1252 | N | GLU | A | 496 | 3506 | 3506 | 3506 | 0 | 0 | 0 | A | N |
| ATOM | 1254 | CA | GLU | A | 496 | −9.609 | 27.027 | 16.947 | 1.00 | 19.02 | | A | C |
| ANISOU | 1254 | CA | GLU | A | 496 | 2418 | 2426 | 2383 | −10 | −8 | 41 | A | C |
| ATOM | 1256 | CB | GLU | A | 496 | −10.924 | 26.542 | 17.542 | 1.00 | 20.20 | | A | C |
| ANISOU | 1256 | CB | GLU | A | 496 | 2589 | 2557 | 2526 | −73 | 0 | 58 | A | C |
| ATOM | 1259 | CG | GLU | A | 496 | −11.154 | 28.033 | 17.552 | 0.00 | 24.82 | | A | C |
| ANISOU | 1259 | CG | GLU | A | 496 | 3143 | 3143 | 3143 | 0 | 0 | 0 | A | C |
| ATOM | 1262 | CD | GLU | A | 496 | −11.168 | 28.880 | 18.814 | 0.00 | 24.31 | | A | C |
| ANISOU | 1262 | CD | GLU | A | 496 | 3079 | 3079 | 3079 | 0 | 0 | 0 | A | C |
| ATOM | 1263 | OE1 | GLU | A | 496 | −10.146 | 29.543 | 19.101 | 0.00 | 24.05 | | A | O |
| ANISOU | 1263 | OE1 | GLU | A | 496 | 3046 | 3046 | 3046 | 0 | 0 | 0 | A | O |
| ATOM | 1264 | OE2 | GLU | A | 496 | −12.203 | 28.889 | 19.513 | 0.00 | 24.05 | | A | O |
| ANISOU | 1264 | OE2 | GLU | A | 496 | 3046 | 3046 | 3046 | 0 | 0 | 0 | A | O |
| ATOM | 1265 | C | GLU | A | 496 | −8.667 | 25.895 | 16.623 | 1.00 | 18.59 | | A | C |
| ANISOU | 1265 | C | GLU | A | 496 | 2385 | 2359 | 2317 | −46 | −12 | 65 | A | C |
| ATOM | 1266 | O | GLU | A | 496 | −8.359 | 24.644 | 15.324 | 0.00 | 25.40 | | A | O |
| ANISOU | 1266 | O | GLU | A | 496 | 3217 | 3217 | 3217 | 0 | 0 | 0 | A | O |
| ATOM | 1267 | N | PRO | A | 497 | −7.493 | 25.950 | 17.245 | 1.00 | 17.85 | | A | N |
| ANISOU | 1267 | N | PRO | A | 497 | 2297 | 2252 | 2230 | −14 | 14 | 39 | A | N |
| ATOM | 1268 | CA | PRO | A | 497 | −6.400 | 25.047 | 16.901 | 1.00 | 17.30 | | A | C |
| ANISOU | 1268 | CA | PRO | A | 497 | 2270 | 2149 | 2152 | −10 | −1 | 39 | A | C |
| ATOM | 1270 | CB | PRO | A | 497 | −5.211 | 25.617 | 17.674 | 1.00 | 17.74 | | A | C |
| ANISOU | 1270 | CB | PRO | A | 497 | 2272 | 2241 | 2225 | −16 | −1 | 22 | A | C |
| ATOM | 1273 | CG | PRO | A | 497 | −5.805 | 26.406 | 18.785 | 1.00 | 18.13 | | A | C |
| ANISOU | 1273 | CG | PRO | A | 497 | 2355 | 2309 | 2225 | −8 | 5 | 28 | A | C |
| ATOM | 1276 | CD | PRO | A | 497 | −7.131 | 26.875 | 18.331 | 1.00 | 17.50 | | A | C |
| ANISOU | 1276 | CD | PRO | A | 497 | 2265 | 2174 | 2209 | 3 | 28 | 8 | A | C |
| ATOM | 1279 | C | PRO | A | 497 | −6.718 | 23.634 | 17.364 | 1.00 | 16.79 | | A | C |
| ANISOU | 1279 | C | PRO | A | 497 | 2219 | 2066 | 2094 | 0 | 11 | −7 | A | C |
| ATOM | 1280 | O | PRO | A | 497 | −7.105 | 23.424 | 18.502 | 1.00 | 15.67 | | A | O |
| ANISOU | 1280 | O | PRO | A | 497 | 2139 | 1856 | 1957 | −7 | −7 | 51 | A | O |
| ATOM | 1281 | N | THR | A | 498 | −6.574 | 22.689 | 16.454 | 1.00 | 16.24 | | A | N |
| ANISOU | 1281 | N | THR | A | 498 | 2193 | 1995 | 1979 | −10 | −12 | 10 | A | N |
| ATOM | 1283 | CA | THR | A | 498 | −6.826 | 21.298 | 16.743 | 1.00 | 16.32 | | A | C |
| ANISOU | 1283 | CA | THR | A | 498 | 2150 | 2031 | 2018 | 15 | −10 | −22 | A | C |
| ATOM | 1285 | CB | THR | A | 498 | −6.645 | 20.528 | 15.461 | 1.00 | 17.01 | | A | C |
| ANISOU | 1285 | CB | THR | A | 498 | 2249 | 2118 | 2095 | 11 | −7 | −62 | A | C |
| ATOM | 1287 | OG1 | THR | A | 498 | −7.585 | 21.017 | 14.504 | 1.00 | 19.42 | | A | O |
| ANISOU | 1287 | OG1 | THR | A | 498 | 2556 | 2625 | 2195 | −5 | −72 | −128 | A | O |
| ATOM | 1289 | CG2 | THR | A | 498 | −6.987 | 19.070 | 15.648 | 1.00 | 18.09 | | A | C |
| ANISOU | 1289 | CG2 | THR | A | 498 | 2409 | 2207 | 2254 | −39 | −60 | 30 | A | C |
| ATOM | 1293 | C | THR | A | 498 | −5.873 | 20.796 | 17.805 | 1.00 | 14.82 | | A | C |
| ANISOU | 1293 | C | THR | A | 498 | 1955 | 1828 | 1845 | −15 | 12 | −22 | A | C |
| ATOM | 1294 | O | THR | A | 498 | −4.679 | 21.065 | 17.728 | 1.00 | 14.45 | | A | O |
| ANISOU | 1294 | O | THR | A | 498 | 1997 | 1650 | 1840 | 9 | 25 | −11 | A | O |
| ATOM | 1295 | N | TRP | A | 499 | −6.413 | 20.095 | 18.808 | 1.00 | 13.64 | | A | N |
| ANISOU | 1295 | N | TRP | A | 499 | 1766 | 1645 | 1770 | 29 | −52 | 20 | A | N |
| ATOM | 1297 | CA | TRP | A | 499 | −5.611 | 19.466 | 19.852 | 1.00 | 12.85 | | A | C |
| ANISOU | 1297 | CA | TRP | A | 499 | 1698 | 1530 | 1654 | −31 | −45 | 49 | A | C |
| ATOM | 1299 | CB | TRP | A | 499 | −5.999 | 19.998 | 21.227 | 1.00 | 11.89 | | A | C |
| ANISOU | 1299 | CB | TRP | A | 499 | 1551 | 1359 | 1605 | 46 | −27 | 74 | A | C |
| ATOM | 1302 | CG | TRP | A | 499 | −5.594 | 21.411 | 21.543 | 1.00 | 10.80 | | A | C |
| ANISOU | 1302 | CG | TRP | A | 499 | 1448 | 1361 | 1293 | −34 | −35 | 59 | A | C |
| ATOM | 1303 | CD1 | TRP | A | 499 | −4.952 | 22.301 | 20.725 | 1.00 | 11.96 | | A | C |
| ANISOU | 1303 | CD1 | TRP | A | 499 | 1697 | 1428 | 1417 | −126 | 10 | −19 | A | C |
| ATOM | 1305 | NE1 | TRP | A | 499 | −4.758 | 23.486 | 21.386 | 1.00 | 10.80 | | A | N |
| ANISOU | 1305 | NE1 | TRP | A | 499 | 1627 | 1194 | 1281 | 16 | −6 | 65 | A | N |
| ATOM | 1307 | CE2 | TRP | A | 499 | −5.270 | 23.389 | 22.647 | 1.00 | 11.62 | | A | C |
| ANISOU | 1307 | CE2 | TRP | A | 499 | 1482 | 1456 | 1476 | 51 | −4 | 68 | A | C |
| ATOM | 1308 | CD2 | TRP | A | 499 | −5.812 | 22.098 | 22.778 | 1.00 | 9.92 | | A | C |
| ANISOU | 1308 | CD2 | TRP | A | 499 | 1272 | 1276 | 1218 | 43 | 8 | 18 | A | C |
| ATOM | 1309 | CE3 | TRP | A | 499 | −6.398 | 21.739 | 24.006 | 1.00 | 11.26 | | A | C |
| ANISOU | 1309 | CE3 | TRP | A | 499 | 1373 | 1445 | 1459 | 0 | 8 | 191 | A | C |
| ATOM | 1311 | CZ3 | TRP | A | 499 | −6.425 | 22.666 | 25.022 | 1.00 | 12.18 | | A | C |
| ANISOU | 1311 | CZ3 | TRP | A | 499 | 1618 | 1691 | 1316 | 27 | 25 | 183 | A | C |
| ATOM | 1313 | CH2 | TRP | A | 499 | −5.902 | 23.958 | 24.858 | 1.00 | 13.93 | | A | C |
| ANISOU | 1313 | CH2 | TRP | A | 499 | 1773 | 1878 | 1639 | −84 | 65 | 20 | A | C |
| ATOM | 1315 | CZ2 | TRP | A | 499 | −5.317 | 24.341 | 23.677 | 1.00 | 12.47 | | A | C |
| ANISOU | 1315 | CZ2 | TRP | A | 499 | 1647 | 1680 | 1409 | −67 | −25 | 32 | A | C |
| ATOM | 1317 | C | TRP | A | 499 | −5.854 | 17.966 | 19.870 | 1.00 | 12.90 | | A | C |
| ANISOU | 1317 | C | TRP | A | 499 | 1673 | 1527 | 1700 | 1 | −22 | 2 | A | C |
| ATOM | 1318 | O | TRP | A | 499 | −7.015 | 17.536 | 19.903 | 1.00 | 14.17 | | A | O |
| ANISOU | 1318 | O | TRP | A | 499 | 1881 | 1504 | 1997 | −34 | −74 | 72 | A | O |
| ATOM | 1319 | N | ILE | A | 500 | −4.768 | 17.194 | 19.886 | 1.00 | 11.88 | | A | N |
| ANISOU | 1319 | N | ILE | A | 500 | 1587 | 1390 | 1535 | −1 | −37 | 46 | A | N |
| ATOM | 1321 | CA | ILE | A | 500 | −4.824 | 15.769 | 20.179 | 1.00 | 12.14 | | A | C |
| ANISOU | 1321 | CA | ILE | A | 500 | 1573 | 1451 | 1589 | −38 | −40 | 21 | A | C |
| ATOM | 1323 | CB | ILE | A | 500 | −4.030 | 14.965 | 19.133 | 1.00 | 13.42 | | A | C |
| ANISOU | 1323 | CB | ILE | A | 500 | 1744 | 1624 | 1731 | −13 | 0 | 21 | A | C |
| ATOM | 1325 | CG1 | ILE | A | 500 | −4.536 | 15.253 | 17.713 | 1.00 | 16.00 | | A | C |
| ANISOU | 1325 | CG1 | ILE | A | 500 | 2127 | 1977 | 1973 | 7 | −56 | 80 | A | C |
| ATOM | 1328 | CD1 | ILE | A | 500 | −6.012 | 15.212 | 17.531 | 1.00 | 18.88 | | A | C |
| ANISOU | 1328 | CD1 | ILE | A | 500 | 2343 | 2457 | 2372 | 15 | 16 | 10 | A | C |
| ATOM | 1332 | CG2 | ILE | A | 500 | −4.094 | 13.472 | 19.419 | 1.00 | 14.86 | | A | C |
| ANISOU | 1332 | CG2 | ILE | A | 500 | 2003 | 1694 | 1946 | −68 | −63 | −14 | A | C |
| ATOM | 1336 | C | ILE | A | 500 | −4.273 | 15.587 | 21.582 | 1.00 | 11.17 | | A | C |
| ANISOU | 1336 | C | ILE | A | 500 | 1417 | 1310 | 1515 | −44 | −42 | 1 | A | C |
| ATOM | 1337 | O | ILE | A | 500 | −3.112 | 15.868 | 21.867 | 1.00 | 11.84 | | A | O |
| ANISOU | 1337 | O | ILE | A | 500 | 1452 | 1347 | 1699 | −110 | −65 | 43 | A | O |
| ATOM | 1338 | N | ILE | A | 501 | −5.129 | 15.142 | 22.476 | 1.00 | 10.69 | | A | N |
| ANISOU | 1338 | N | ILE | A | 501 | 1402 | 1215 | 1443 | −11 | −47 | 16 | A | N |
| ATOM | 1340 | CA | ILE | A | 501 | −4.804 | 15.065 | 23.883 | 1.00 | 10.07 | | A | C |
| ANISOU | 1340 | CA | ILE | A | 501 | 1289 | 1167 | 1371 | −33 | −41 | −1 | A | C |
| ATOM | 1342 | CB | ILE | A | 501 | −6.002 | 15.548 | 24.757 | 1.00 | 10.35 | | A | C |
| ANISOU | 1342 | CB | ILE | A | 501 | 1289 | 1232 | 1409 | −19 | −22 | 10 | A | C |
| ATOM | 1344 | CG1 | ILE | A | 501 | −6.453 | 16.956 | 24.336 | 1.00 | 11.96 | | A | C |
| ANISOU | 1344 | CG1 | ILE | A | 501 | 1570 | 1393 | 1580 | −13 | −59 | 22 | A | C |
| ATOM | 1347 | CD1 | ILE | A | 501 | −7.847 | 17.192 | 24.617 | 1.00 | 13.84 | | A | C |
| ANISOU | 1347 | CD1 | ILE | A | 501 | 1751 | 1603 | 1904 | 63 | −39 | 6 | A | C |
| ATOM | 1351 | CG2 | ILE | A | 501 | −5.657 | 15.564 | 26.216 | 1.00 | 9.55 | | A | C |
| ANISOU | 1351 | CG2 | ILE | A | 501 | 1196 | 1033 | 1397 | 99 | 36 | −76 | A | C |
| ATOM | 1355 | C | ILE | A | 501 | −4.426 | 13.624 | 24.187 | 1.00 | 10.31 | | A | C |
| ANISOU | 1355 | C | ILE | A | 501 | 1320 | 1158 | 1437 | −38 | −41 | 16 | A | C |
| ATOM | 1356 | O | ILE | A | 501 | −5.255 | 12.719 | 24.059 | 1.00 | 10.06 | | A | O |
| ANISOU | 1356 | O | ILE | A | 501 | 1331 | 1022 | 1467 | −98 | 15 | 16 | A | O |
| ATOM | 1357 | N | MET | A | 502 | −3.167 | 13.435 | 24.568 | 1.00 | 10.54 | | A | N |
| ANISOU | 1357 | N | MET | A | 502 | 1331 | 1201 | 1471 | −4 | −2 | 32 | A | N |
| ATOM | 1359 | CA | MET | A | 502 | −2.621 | 12.134 | 24.964 | 1.00 | 11.99 | | A | C |
| ANISOU | 1359 | CA | MET | A | 502 | 1590 | 1341 | 1622 | 21 | −10 | −7 | A | C |
| ATOM | 1361 | CB | MET | A | 502 | −1.303 | 11.888 | 24.242 | 1.00 | 13.19 | | A | C |
| ANISOU | 1361 | CB | MET | A | 502 | 1721 | 1540 | 1750 | 37 | 41 | 38 | A | C |
| ATOM | 1364 | CG | MET | A | 502 | −1.346 | 11.998 | 22.722 | 1.00 | 17.72 | | A | C |
| ANISOU | 1364 | CG | MET | A | 502 | 2343 | 2292 | 2098 | 42 | −40 | 50 | A | C |
| ATOM | 1367 | SD | MET | A | 502 | −2.388 | 10.754 | 21.972 | 1.00 | 20.40 | | A | S |
| ANISOU | 1367 | SD | MET | A | 502 | 2893 | 2505 | 2353 | 65 | −156 | −87 | A | S |
| ATOM | 1368 | CE | MET | A | 502 | −1.484 | 9.269 | 22.260 | 1.00 | 21.03 | | A | C |
| ANISOU | 1368 | CE | MET | A | 502 | 2677 | 2560 | 2750 | 24 | −24 | −11 | A | C |
| ATOM | 1372 | C | MET | A | 502 | −2.324 | 12.079 | 26.447 | 1.00 | 11.47 | | A | C |
| ANISOU | 1372 | C | MET | A | 502 | 1448 | 1318 | 1590 | 59 | 7 | −13 | A | C |
| ATOM | 1373 | O | MET | A | 502 | −2.052 | 13.086 | 27.078 | 1.00 | 10.71 | | A | O |
| ANISOU | 1373 | O | MET | A | 502 | 1324 | 1276 | 1467 | 40 | 46 | −165 | A | O |
| ATOM | 1374 | N | GLU | A | 503 | −2.325 | 10.884 | 27.007 | 1.00 | 11.37 | | A | N |
| ANISOU | 1374 | N | GLU | A | 503 | 1479 | 1262 | 1578 | 38 | 21 | −36 | A | N |
| ATOM | 1376 | CA | GLU | A | 503 | −1.693 | 10.661 | 28.304 | 1.00 | 13.03 | | A | C |
| ANISOU | 1376 | CA | GLU | A | 503 | 1642 | 1622 | 1684 | 59 | −6 | −29 | A | C |
| ATOM | 1378 | CB | GLU | A | 503 | −1.809 | 9.175 | 28.658 | 1.00 | 14.57 | | A | C |
| ANISOU | 1378 | CB | GLU | A | 503 | 1822 | 1742 | 1972 | −3 | −12 | −11 | A | C |
| ATOM | 1381 | CG | GLU | A | 503 | −1.514 | 8.790 | 30.099 | 1.00 | 19.60 | | A | C |
| ANISOU | 1381 | CG | GLU | A | 503 | 2498 | 2570 | 2377 | 2 | −70 | 56 | A | C |
| ATOM | 1384 | CD | GLU | A | 503 | −1.930 | 7.350 | 30.408 | 1.00 | 23.91 | | A | C |
| ANISOU | 1384 | CD | GLU | A | 503 | 3150 | 2793 | 3142 | −3 | −26 | 83 | A | C |
| ATOM | 1385 | OE1 | GLU | A | 503 | −1.691 | 6.447 | 29.580 | 1.00 | 27.71 | | A | O |
| ANISOU | 1385 | OE1 | GLU | A | 503 | 3785 | 3287 | 3455 | −78 | −100 | −74 | A | O |
| ATOM | 1386 | OE2 | GLU | A | 503 | −2.491 | 7.109 | 31.485 | 1.00 | 28.61 | | A | O |
| ANISOU | 1386 | OE2 | GLU | A | 503 | 3684 | 3605 | 3578 | −13 | 127 | 58 | A | O |
| ATOM | 1387 | C | GLU | A | 503 | −0.220 | 11.115 | 28.273 | 1.00 | 13.12 | | A | C |
| ANISOU | 1387 | C | GLU | A | 503 | 1637 | 1676 | 1670 | 61 | −22 | −28 | A | C |
| ATOM | 1388 | O | GLU | A | 503 | 0.480 | 10.902 | 27.277 | 1.00 | 14.04 | | A | O |
| ANISOU | 1388 | O | GLU | A | 503 | 1672 | 1832 | 1830 | 179 | −63 | −63 | A | O |
| ATOM | 1389 | N | LEU | A | 504 | 0.225 | 11.793 | 29.338 | 1.00 | 12.78 | | A | N |
| ANISOU | 1389 | N | LEU | A | 504 | 1560 | 1617 | 1679 | 107 | −26 | −59 | A | N |
| ATOM | 1391 | CA | LEU | A | 504 | 1.621 | 12.220 | 29.470 | 1.00 | 13.70 | | A | C |
| ANISOU | 1391 | CA | LEU | A | 504 | 1729 | 1724 | 1751 | 55 | −17 | 0 | A | C |
| ATOM | 1393 | CB | LEU | A | 504 | 1.723 | 13.411 | 30.433 | 1.00 | 13.89 | | A | C |
| ANISOU | 1393 | CB | LEU | A | 504 | 1753 | 1720 | 1804 | 101 | −35 | −24 | A | C |
| ATOM | 1396 | CG | LEU | A | 504 | 3.133 | 13.978 | 30.629 | 1.00 | 15.44 | | A | C |
| ANISOU | 1396 | CG | LEU | A | 504 | 1938 | 1908 | 2020 | 14 | −12 | −49 | A | C |
| ATOM | 1398 | CD1 | LEU | A | 504 | 3.589 | 14.615 | 29.325 | 1.00 | 17.03 | | A | C |
| ANISOU | 1398 | CD1 | LEU | A | 504 | 2149 | 2227 | 2094 | −11 | 25 | 24 | A | C |
| ATOM | 1402 | CD2 | LEU | A | 504 | 3.177 | 14.991 | 31.799 | 1.00 | 16.68 | | A | C |
| ANISOU | 1402 | CD2 | LEU | A | 504 | 2189 | 1996 | 2152 | −5 | 21 | −80 | A | C |
| ATOM | 1406 | C | LEU | A | 504 | 2.447 | 11.061 | 30.018 | 1.00 | 14.74 | | A | C |
| ANISOU | 1406 | C | LEU | A | 504 | 1804 | 1920 | 1874 | 53 | −50 | 56 | A | C |
| ATOM | 1407 | O | LEU | A | 504 | 2.068 | 10.438 | 31.003 | 1.00 | 15.36 | | A | O |
| ANISOU | 1407 | O | LEU | A | 504 | 1757 | 2086 | 1992 | 189 | 6 | 211 | A | O |
| ATOM | 1408 | N | TYR | A | 505 | 3.566 | 10.793 | 29.363 | 1.00 | 15.03 | | A | N |
| ANISOU | 1408 | N | TYR | A | 505 | 1874 | 1945 | 1889 | 159 | −46 | 44 | A | N |
| ATOM | 1410 | CA | TYR | A | 505 | 4.542 | 9.782 | 29.780 | 1.00 | 16.40 | | A | C |
| ANISOU | 1410 | CA | TYR | A | 505 | 2080 | 2063 | 2087 | 135 | −35 | 45 | A | C |
| ATOM | 1412 | CB | TYR | A | 505 | 4.712 | 8.775 | 28.642 | 1.00 | 16.92 | | A | C |
| ANISOU | 1412 | CB | TYR | A | 505 | 2164 | 2116 | 2148 | 181 | −22 | 14 | A | C |
| ATOM | 1415 | CG | TYR | A | 505 | 3.387 | 8.093 | 28.346 | 1.00 | 18.81 | | A | C |
| ANISOU | 1415 | CG | TYR | A | 505 | 2304 | 2428 | 2414 | 158 | −32 | −7 | A | C |
| ATOM | 1416 | CD1 | TYR | A | 505 | 2.721 | 7.364 | 29.326 | 1.00 | 20.24 | | A | C |
| ANISOU | 1416 | CD1 | TYR | A | 505 | 2509 | 2451 | 2728 | 97 | −78 | 115 | A | C |
| ATOM | 1418 | CE1 | TYR | A | 505 | 1.484 | 6.776 | 29.075 | 1.00 | 21.60 | | A | C |
| ANISOU | 1418 | CE1 | TYR | A | 505 | 2771 | 2650 | 2784 | 6 | −144 | 53 | A | C |
| ATOM | 1420 | CZ | TYR | A | 505 | 0.893 | 6.938 | 27.824 | 1.00 | 19.04 | | A | C |
| ANISOU | 1420 | CZ | TYR | A | 505 | 2563 | 2102 | 2568 | 192 | −66 | −18 | A | C |
| ATOM | 1421 | OH | TYR | A | 505 | −0.337 | 6.366 | 27.585 | 1.00 | 20.41 | | A | O |
| ANISOU | 1421 | OH | TYR | A | 505 | 2876 | 1975 | 2900 | 241 | −198 | −48 | A | O |
| ATOM | 1423 | CE2 | TYR | A | 505 | 1.528 | 7.670 | 26.856 | 1.00 | 19.06 | | A | C |
| ANISOU | 1423 | CE2 | TYR | A | 505 | 2405 | 2382 | 2454 | 196 | −26 | −86 | A | C |
| ATOM | 1425 | CD2 | TYR | A | 505 | 2.746 | 8.264 | 27.121 | 1.00 | 19.86 | | A | C |
| ANISOU | 1425 | CD2 | TYR | A | 505 | 2525 | 2624 | 2396 | 152 | 1 | −144 | A | C |
| ATOM | 1427 | C | TYR | A | 505 | 5.812 | 10.535 | 30.149 | 1.00 | 16.38 | | A | C |
| ANISOU | 1427 | C | TYR | A | 505 | 2097 | 2053 | 2074 | 197 | −44 | 17 | A | C |
| ATOM | 1428 | O | TYR | A | 505 | 6.641 | 10.855 | 29.316 | 1.00 | 16.56 | | A | O |
| ANISOU | 1428 | O | TYR | A | 505 | 2150 | 1986 | 2155 | 248 | −51 | 99 | A | O |
| ATOM | 1429 | N | PRO | A | 506 | 5.924 | 10.899 | 31.420 | 1.00 | 17.54 | | A | N |
| ANISOU | 1429 | N | PRO | A | 506 | 2179 | 2305 | 2178 | 148 | −26 | 35 | A | N |
| ATOM | 1430 | CA | PRO | A | 506 | 6.936 | 11.865 | 31.844 | 1.00 | 17.08 | | A | C |
| ANISOU | 1430 | CA | PRO | A | 506 | 2193 | 2166 | 2131 | 118 | 10 | −15 | A | C |
| ATOM | 1432 | CB | PRO | A | 506 | 6.574 | 12.136 | 33.306 | 1.00 | 17.97 | | A | C |
| ANISOU | 1432 | CB | PRO | A | 506 | 2330 | 2327 | 2169 | 91 | 32 | 20 | A | C |
| ATOM | 1435 | CG | PRO | A | 506 | 5.824 | 10.925 | 33.738 | 1.00 | 17.80 | | A | C |
| ANISOU | 1435 | CG | PRO | A | 506 | 2275 | 2259 | 2227 | 44 | 3 | −115 | A | C |
| ATOM | 1438 | CD | PRO | A | 506 | 5.110 | 10.381 | 32.534 | 1.00 | 17.74 | | A | C |
| ANISOU | 1438 | CD | PRO | A | 506 | 2230 | 2321 | 2187 | 100 | −56 | 30 | A | C |
| ATOM | 1441 | C | PRO | A | 506 | 8.384 | 11.346 | 31.734 | 1.00 | 16.27 | | A | C |
| ANISOU | 1441 | C | PRO | A | 506 | 2071 | 1999 | 2111 | 54 | 15 | −32 | A | C |
| ATOM | 1442 | O | PRO | A | 506 | 9.316 | 12.131 | 31.656 | 1.00 | 16.69 | | A | O |
| ANISOU | 1442 | O | PRO | A | 506 | 2253 | 1902 | 2185 | 47 | −19 | −77 | A | O |
| ATOM | 1443 | N | TYR | A | 507 | 8.567 | 10.031 | 31.721 | 1.00 | 14.24 | | A | N |
| ANISOU | 1443 | N | TYR | A | 507 | 1798 | 1774 | 1838 | 46 | −25 | −65 | A | N |
| ATOM | 1445 | CA | TYR | A | 507 | 9.904 | 9.477 | 31.587 | 1.00 | 12.77 | | A | C |
| ANISOU | 1445 | CA | TYR | A | 507 | 1591 | 1593 | 1668 | 10 | −11 | −38 | A | C |
| ATOM | 1447 | CB | TYR | A | 507 | 9.964 | 8.074 | 32.166 | 1.00 | 12.48 | | A | C |
| ANISOU | 1447 | CB | TYR | A | 507 | 1465 | 1590 | 1687 | 25 | 25 | −63 | A | C |
| ATOM | 1450 | CG | TYR | A | 507 | 9.736 | 8.018 | 33.654 | 1.00 | 12.63 | | A | C |
| ANISOU | 1450 | CG | TYR | A | 507 | 1612 | 1485 | 1700 | 17 | −50 | 1 | A | C |
| ATOM | 1451 | CD1 | TYR | A | 507 | 10.786 | 8.202 | 34.542 | 1.00 | 13.86 | | A | C |
| ANISOU | 1451 | CD1 | TYR | A | 507 | 1656 | 1856 | 1754 | 42 | −26 | −110 | A | C |
| ATOM | 1453 | CE1 | TYR | A | 507 | 10.589 | 8.151 | 35.916 | 1.00 | 15.19 | | A | C |
| ANISOU | 1453 | CE1 | TYR | A | 507 | 1910 | 2047 | 1812 | 28 | −16 | −35 | A | C |
| ATOM | 1455 | CZ | TYR | A | 507 | 9.339 | 7.892 | 36.415 | 1.00 | 16.05 | | A | C |
| ANISOU | 1455 | CZ | TYR | A | 507 | 1938 | 2236 | 1923 | −43 | −3 | −81 | A | C |
| ATOM | 1456 | OH | TYR | A | 507 | 9.173 | 7.852 | 37.775 | 1.00 | 19.87 | | A | O |
| ANISOU | 1456 | OH | TYR | A | 507 | 2688 | 2691 | 2169 | −70 | −3 | −15 | A | O |
| ATOM | 1458 | CE2 | TYR | A | 507 | 8.266 | 7.707 | 35.554 | 1.00 | 14.97 | | A | C |
| ANISOU | 1458 | CE2 | TYR | A | 507 | 1915 | 1955 | 1818 | −37 | −5 | 46 | A | C |
| ATOM | 1460 | CD2 | TYR | A | 507 | 8.480 | 7.754 | 34.175 | 1.00 | 12.99 | | A | C |
| ANISOU | 1460 | CD2 | TYR | A | 507 | 1623 | 1582 | 1731 | 60 | 24 | −80 | A | C |
| ATOM | 1462 | C | TYR | A | 507 | 10.395 | 9.481 | 30.148 | 1.00 | 11.87 | | A | C |
| ANISOU | 1462 | C | TYR | A | 507 | 1497 | 1402 | 1609 | 22 | −49 | −37 | A | C |
| ATOM | 1463 | O | TYR | A | 507 | 11.570 | 9.211 | 29.900 | 1.00 | 12.76 | | A | O |
| ANISOU | 1463 | O | TYR | A | 507 | 1543 | 1492 | 1813 | 100 | −1 | −13 | A | O |
| ATOM | 1464 | N | GLY | A | 508 | 9.517 | 9.776 | 29.187 | 1.00 | 11.45 | | A | N |
| ANISOU | 1464 | N | GLY | A | 508 | 1455 | 1342 | 1553 | 90 | −23 | −75 | A | N |
| ATOM | 1466 | CA | GLY | A | 508 | 9.926 | 9.887 | 27.811 | 1.00 | 11.62 | | A | C |
| ANISOU | 1466 | CA | GLY | A | 508 | 1501 | 1371 | 1542 | 35 | −41 | −35 | A | C |
| ATOM | 1469 | C | GLY | A | 508 | 10.173 | 8.594 | 27.050 | 1.00 | 11.14 | | A | C |
| ANISOU | 1469 | C | GLY | A | 508 | 1422 | 1325 | 1484 | 52 | 52 | −20 | A | C |
| ATOM | 1470 | O | GLY | A | 508 | 9.661 | 7.541 | 27.396 | 1.00 | 10.63 | | A | O |
| ANISOU | 1470 | O | GLY | A | 508 | 1361 | 1324 | 1351 | 48 | 121 | −26 | A | O |
| ATOM | 1471 | N | GLU | A | 509 | 10.939 | 8.721 | 25.967 | 1.00 | 10.48 | | A | N |
| ANISOU | 1471 | N | GLU | A | 509 | 1444 | 1200 | 1338 | 52 | 61 | −37 | A | N |
| ATOM | 1473 | CA | GLU | A | 509 | 11.240 | 7.585 | 25.103 | 1.00 | 11.08 | | A | C |
| ANISOU | 1473 | CA | GLU | A | 509 | 1457 | 1332 | 1420 | 44 | −9 | −59 | A | C |
| ATOM | 1475 | CB | GLU | A | 509 | 11.999 | 8.040 | 23.875 | 1.00 | 11.95 | | A | C |
| ANISOU | 1475 | CB | GLU | A | 509 | 1574 | 1429 | 1536 | 22 | 27 | −6 | A | C |
| ATOM | 1478 | CG | GLU | A | 509 | 11.176 | 8.934 | 22.952 | 1.00 | 13.26 | | A | C |
| ANISOU | 1478 | CG | GLU | A | 509 | 1705 | 1599 | 1731 | 104 | −17 | 34 | A | C |
| ATOM | 1481 | CD | GLU | A | 509 | 11.969 | 9.497 | 21.781 | 1.00 | 16.05 | | A | C |
| ANISOU | 1481 | CD | GLU | A | 509 | 2080 | 2080 | 1937 | 21 | 59 | 45 | A | C |
| ATOM | 1482 | OE1 | GLU | A | 509 | 13.142 | 9.112 | 21.572 | 1.00 | 16.36 | | A | O |
| ANISOU | 1482 | OE1 | GLU | A | 509 | 2205 | 1850 | 2158 | 63 | 18 | 99 | A | O |
| ATOM | 1483 | OE2 | GLU | A | 509 | 11.397 | 10.356 | 21.073 | 1.00 | 20.02 | | A | O |
| ANISOU | 1483 | OE2 | GLU | A | 509 | 2779 | 2307 | 2520 | 164 | 27 | 169 | A | O |
| ATOM | 1484 | C | GLU | A | 509 | 12.060 | 6.531 | 25.806 | 1.00 | 10.19 | | A | C |
| ANISOU | 1484 | C | GLU | A | 509 | 1312 | 1249 | 1308 | −7 | −26 | −37 | A | C |
| ATOM | 1485 | O | GLU | A | 509 | 12.972 | 6.850 | 26.552 | 1.00 | 11.22 | | A | O |
| ANISOU | 1485 | O | GLU | A | 509 | 1438 | 1460 | 1365 | 163 | −105 | −66 | A | O |
| ATOM | 1486 | N | LEU | A | 510 | 11.781 | 5.273 | 25.494 | 1.00 | 10.30 | | A | N |
| ANISOU | 1486 | N | LEU | A | 510 | 1356 | 1268 | 1288 | 57 | −39 | −60 | A | N |
| ATOM | 1488 | CA | LEU | A | 510 | 12.486 | 4.176 | 26.098 | 1.00 | 10.07 | | A | C |
| ANISOU | 1488 | CA | LEU | A | 510 | 1303 | 1288 | 1235 | 11 | 14 | −18 | A | C |
| ATOM | 1490 | CB | LEU | A | 510 | 11.839 | 2.836 | 25.722 | 1.00 | 10.20 | | A | C |
| ANISOU | 1490 | CB | LEU | A | 510 | 1374 | 1251 | 1249 | −11 | 72 | −9 | A | C |
| ATOM | 1493 | CG | LEU | A | 510 | 12.436 | 1.566 | 26.308 | 1.00 | 9.60 | | A | C |
| ANISOU | 1493 | CG | LEU | A | 510 | 1103 | 1347 | 1194 | 38 | 67 | −38 | A | C |
| ATOM | 1495 | CD1 | LEU | A | 510 | 12.459 | 1.659 | 27.841 | 1.00 | 9.22 | | A | C |
| ANISOU | 1495 | CD1 | LEU | A | 510 | 1211 | 1114 | 1175 | 88 | 55 | −61 | A | C |
| ATOM | 1499 | CD2 | LEU | A | 510 | 11.682 | 0.370 | 25.821 | 1.00 | 10.25 | | A | C |
| ANISOU | 1499 | CD2 | LEU | A | 510 | 1325 | 1204 | 1363 | 90 | 110 | −80 | A | C |
| ATOM | 1503 | C | LEU | A | 510 | 13.968 | 4.163 | 25.765 | 1.00 | 9.99 | | A | C |
| ANISOU | 1503 | C | LEU | A | 510 | 1312 | 1249 | 1235 | 22 | 49 | −29 | A | C |
| ATOM | 1504 | O | LEU | A | 510 | 14.760 | 3.823 | 26.632 | 1.00 | 9.82 | | A | O |
| ANISOU | 1504 | O | LEU | A | 510 | 1364 | 1174 | 1193 | 11 | 80 | −97 | A | O |
| ATOM | 1505 | N | GLY | A | 511 | 14.357 | 4.502 | 24.532 | 1.00 | 10.34 | | A | N |
| ANISOU | 1505 | N | GLY | A | 511 | 1267 | 1323 | 1335 | −4 | 40 | −52 | A | N |
| ATOM | 1507 | CA | GLY | A | 511 | 15.773 | 4.432 | 24.191 | 1.00 | 10.19 | | A | C |
| ANISOU | 1507 | CA | GLY | A | 511 | 1240 | 1269 | 1359 | −54 | −1 | −36 | A | C |
| ATOM | 1510 | C | GLY | A | 511 | 16.598 | 5.311 | 25.121 | 1.00 | 10.62 | | A | C |
| ANISOU | 1510 | C | GLY | A | 511 | 1302 | 1345 | 1387 | −21 | −8 | −10 | A | C |
| ATOM | 1511 | O | GLY | A | 511 | 17.570 | 4.853 | 25.744 | 1.00 | 10.45 | | A | O |
| ANISOU | 1511 | O | GLY | A | 511 | 1160 | 1348 | 1460 | −17 | −4 | −14 | A | O |
| ATOM | 1512 | N | HIS | A | 512 | 16.194 | 6.563 | 25.238 | 1.00 | 10.65 | | A | N |
| ANISOU | 1512 | N | HIS | A | 512 | 1316 | 1418 | 1312 | −22 | −1 | −29 | A | N |
| ATOM | 1514 | CA | HIS | A | 512 | 16.915 | 7.499 | 26.076 | 1.00 | 11.18 | | A | C |
| ANISOU | 1514 | CA | HIS | A | 512 | 1376 | 1430 | 1442 | −50 | −39 | −60 | A | C |
| ATOM | 1516 | CB | HIS | A | 512 | 16.422 | 8.925 | 25.835 | 1.00 | 12.25 | | A | C |
| ANISOU | 1516 | CB | HIS | A | 512 | 1527 | 1468 | 1657 | −15 | −28 | −80 | A | C |
| ATOM | 1519 | CG | HIS | A | 512 | 16.638 | 9.391 | 24.428 | 1.00 | 17.53 | | A | C |
| ANISOU | 1519 | CG | HIS | A | 512 | 2421 | 2148 | 2089 | −185 | −8 | −1 | A | C |
| ATOM | 1520 | ND1 | HIS | A | 512 | 16.129 | 10.570 | 23.938 | 1.00 | 24.90 | | A | N |
| ANISOU | 1520 | ND1 | HIS | A | 512 | 3433 | 2964 | 3063 | 182 | 39 | 146 | A | N |
| ATOM | 1522 | CE1 | HIS | A | 512 | 16.471 | 10.709 | 22.668 | 1.00 | 24.85 | | A | C |
| ANISOU | 1522 | CE1 | HIS | A | 512 | 3320 | 3104 | 3017 | 91 | 17 | 46 | A | C |
| ATOM | 1524 | NE2 | HIS | A | 512 | 17.154 | 9.644 | 22.303 | 1.00 | 24.72 | | A | N |
| ANISOU | 1524 | NE2 | HIS | A | 512 | 3427 | 2884 | 3081 | −99 | 130 | 124 | A | N |
| ATOM | 1526 | CD2 | HIS | A | 512 | 17.266 | 8.800 | 23.384 | 1.00 | 23.85 | | A | C |
| ANISOU | 1526 | CD2 | HIS | A | 512 | 3241 | 2958 | 2863 | 175 | 153 | −11 | A | C |
| ATOM | 1528 | C | HIS | A | 512 | 16.830 | 7.106 | 27.555 | 1.00 | 10.37 | | A | C |
| ANISOU | 1528 | C | HIS | A | 512 | 1271 | 1317 | 1350 | −53 | 29 | −64 | A | C |
| ATOM | 1529 | O | HIS | A | 512 | 17.811 | 7.238 | 28.288 | 1.00 | 10.94 | | A | O |
| ANISOU | 1529 | O | HIS | A | 512 | 1404 | 1252 | 1500 | −17 | 0 | −185 | A | O |
| ATOM | 1530 | N | TYR | A | 513 | 15.683 | 6.581 | 27.978 | 1.00 | 9.37 | | A | N |
| ANISOU | 1530 | N | TYR | A | 513 | 1234 | 1083 | 1240 | 37 | 36 | −82 | A | N |
| ATOM | 1532 | CA | TYR | A | 513 | 15.521 | 6.095 | 29.335 | 1.00 | 9.93 | | A | C |
| ANISOU | 1532 | CA | TYR | A | 513 | 1269 | 1297 | 1206 | 32 | 30 | −86 | A | C |
| ATOM | 1534 | CB | TYR | A | 513 | 14.094 | 5.612 | 29.532 | 1.00 | 9.27 | | A | C |
| ANISOU | 1534 | CB | TYR | A | 513 | 1215 | 1221 | 1084 | 43 | 63 | −47 | A | C |
| ATOM | 1537 | CG | TYR | A | 513 | 13.766 | 5.046 | 30.887 | 1.00 | 8.67 | | A | C |
| ANISOU | 1537 | CG | TYR | A | 513 | 1183 | 1067 | 1044 | 65 | 117 | −28 | A | C |
| ATOM | 1538 | CD1 | TYR | A | 513 | 13.205 | 5.856 | 31.881 | 1.00 | 9.29 | | A | C |
| ANISOU | 1538 | CD1 | TYR | A | 513 | 1235 | 1104 | 1191 | −58 | 12 | −129 | A | C |
| ATOM | 1540 | CE1 | TYR | A | 513 | 12.882 | 5.336 | 33.110 | 1.00 | 10.43 | | A | C |
| ANISOU | 1540 | CE1 | TYR | A | 513 | 1344 | 1343 | 1275 | −3 | −28 | −115 | A | C |
| ATOM | 1542 | CZ | TYR | A | 513 | 13.064 | 4.002 | 33.362 | 1.00 | 10.54 | | A | C |
| ANISOU | 1542 | CZ | TYR | A | 513 | 1127 | 1426 | 1449 | −42 | 28 | 66 | A | C |
| ATOM | 1543 | OH | TYR | A | 513 | 12.721 | 3.521 | 34.590 | 1.00 | 11.85 | | A | O |
| ANISOU | 1543 | OH | TYR | A | 513 | 1458 | 1688 | 1356 | −15 | 115 | −81 | A | O |
| ATOM | 1545 | CE2 | TYR | A | 513 | 13.616 | 3.167 | 32.409 | 1.00 | 10.42 | | A | C |
| ANISOU | 1545 | CE2 | TYR | A | 513 | 1323 | 1221 | 1415 | −124 | 61 | 61 | A | C |
| ATOM | 1547 | CD2 | TYR | A | 513 | 13.946 | 3.690 | 31.158 | 1.00 | 11.11 | | A | C |
| ANISOU | 1547 | CD2 | TYR | A | 513 | 1515 | 1370 | 1334 | 137 | 111 | 53 | A | C |
| ATOM | 1549 | C | TYR | A | 513 | 16.518 | 4.970 | 29.634 | 1.00 | 9.64 | | A | C |
| ANISOU | 1549 | C | TYR | A | 513 | 1285 | 1214 | 1162 | 56 | −8 | −54 | A | C |
| ATOM | 1550 | O | TYR | A | 513 | 17.173 | 4.985 | 30.673 | 1.00 | 9.60 | | A | O |
| ANISOU | 1550 | O | TYR | A | 513 | 1340 | 1279 | 1028 | 69 | 69 | −97 | A | O |
| ATOM | 1551 | N | LEU | A | 514 | 16.675 | 4.010 | 28.717 | 1.00 | 9.19 | | A | N |
| ANISOU | 1551 | N | LEU | A | 514 | 1287 | 1254 | 951 | 27 | −69 | −75 | A | N |
| ATOM | 1553 | CA | LEU | A | 514 | 17.637 | 2.925 | 28.906 | 1.00 | 9.98 | | A | C |
| ANISOU | 1553 | CA | LEU | A | 514 | 1279 | 1317 | 1195 | 18 | 43 | −75 | A | C |
| ATOM | 1555 | CB | LEU | A | 514 | 17.524 | 1.887 | 27.779 | 1.00 | 10.88 | | A | C |
| ANISOU | 1555 | CB | LEU | A | 514 | 1389 | 1325 | 1417 | 74 | 37 | −134 | A | C |
| ATOM | 1558 | CG | LEU | A | 514 | 16.189 | 1.150 | 27.715 | 1.00 | 11.46 | | A | C |
| ANISOU | 1558 | CG | LEU | A | 514 | 1449 | 1429 | 1476 | 56 | 37 | −113 | A | C |
| ATOM | 1560 | CD1 | LEU | A | 514 | 16.101 | 0.400 | 26.403 | 1.00 | 13.32 | | A | C |
| ANISOU | 1560 | CD1 | LEU | A | 514 | 1685 | 1796 | 1579 | 34 | −17 | −191 | A | C |
| ATOM | 1564 | CD2 | LEU | A | 514 | 16.002 | 0.211 | 28.866 | 1.00 | 13.98 | | A | C |
| ANISOU | 1564 | CD2 | LEU | A | 514 | 1802 | 1662 | 1848 | −10 | −52 | 33 | A | C |
| ATOM | 1568 | C | LEU | A | 514 | 19.068 | 3.440 | 29.001 | 1.00 | 10.78 | | A | C |
| ANISOU | 1568 | C | LEU | A | 514 | 1372 | 1373 | 1350 | 44 | 17 | −91 | A | C |
| ATOM | 1569 | O | LEU | A | 514 | 19.854 | 2.962 | 29.807 | 1.00 | 10.98 | | A | O |
| ANISOU | 1569 | O | LEU | A | 514 | 1302 | 1545 | 1323 | 38 | 33 | −120 | A | O |
| ATOM | 1570 | N | GLU | A | 515 | 19.393 | 4.405 | 28.162 | 1.00 | 11.53 | | A | N |
| ANISOU | 1570 | N | GLU | A | 515 | 1466 | 1504 | 1409 | 18 | 52 | −33 | A | N |
| ATOM | 1572 | CA | GLU | A | 515 | 20.722 | 5.000 | 28.158 | 1.00 | 13.11 | | A | C |
| ANISOU | 1572 | CA | GLU | A | 515 | 1605 | 1704 | 1672 | −13 | −9 | −45 | A | C |
| ATOM | 1574 | CB | GLU | A | 515 | 20.810 | 6.059 | 27.065 | 1.00 | 14.02 | | A | C |
| ANISOU | 1574 | CB | GLU | A | 515 | 1652 | 1837 | 1837 | −26 | 16 | 32 | A | C |
| ATOM | 1577 | CG | GLU | A | 515 | 20.845 | 5.572 | 25.630 | 1.00 | 17.06 | | A | C |
| ANISOU | 1577 | CG | GLU | A | 515 | 2083 | 2245 | 2154 | −72 | 7 | −89 | A | C |
| ATOM | 1580 | CD | GLU | A | 515 | 20.592 | 6.709 | 24.629 | 1.00 | 21.59 | | A | C |
| ANISOU | 1580 | CD | GLU | A | 515 | 2890 | 2657 | 2654 | −41 | −32 | 123 | A | C |
| ATOM | 1581 | OE1 | GLU | A | 515 | 20.608 | 7.895 | 25.042 | 1.00 | 27.20 | | A | O |
| ANISOU | 1581 | OE1 | GLU | A | 515 | 3781 | 3229 | 3325 | 69 | 29 | −96 | A | O |
| ATOM | 1582 | OE2 | GLU | A | 515 | 20.384 | 6.444 | 23.418 | 1.00 | 24.09 | | A | O |
| ANISOU | 1582 | OE2 | GLU | A | 515 | 3216 | 3100 | 2834 | 12 | −27 | −6 | A | O |
| ATOM | 1583 | C | GLU | A | 515 | 21.037 | 5.637 | 29.521 | 1.00 | 13.80 | | A | C |
| ANISOU | 1583 | C | GLU | A | 515 | 1732 | 1775 | 1734 | −43 | −47 | −10 | A | C |
| ATOM | 1584 | O | GLU | A | 515 | 22.100 | 5.366 | 30.096 | 1.00 | 15.50 | | A | O |
| ANISOU | 1584 | O | GLU | A | 515 | 1837 | 2077 | 1972 | −22 | −48 | −3 | A | O |
| ATOM | 1585 | N | ARG | A | 516 | 20.106 | 6.452 | 30.027 | 1.00 | 13.54 | | A | N |
| ANISOU | 1585 | N | ARG | A | 516 | 1705 | 1782 | 1656 | −18 | −88 | −56 | A | N |
| ATOM | 1587 | CA | ARG | A | 516 | 20.250 | 7.179 | 31.311 | 1.00 | 14.19 | | A | C |
| ANISOU | 1587 | CA | ARG | A | 516 | 1795 | 1863 | 1730 | −5 | −61 | −64 | A | C |
| ATOM | 1589 | CB | ARG | A | 516 | 18.972 | 7.991 | 31.640 | 1.00 | 15.29 | | A | C |
| ANISOU | 1589 | CB | ARG | A | 516 | 2017 | 1903 | 1889 | 37 | −24 | −67 | A | C |
| ATOM | 1592 | CG | ARG | A | 516 | 18.762 | 9.181 | 30.826 | 1.00 | 17.75 | | A | C |
| ANISOU | 1592 | CG | ARG | A | 516 | 2275 | 2241 | 2228 | 44 | −47 | 12 | A | C |
| ATOM | 1595 | CD | ARG | A | 516 | 17.877 | 10.195 | 31.461 | 1.00 | 17.62 | | A | C |
| ANISOU | 1595 | CD | ARG | A | 516 | 2249 | 2185 | 2259 | −5 | 81 | −18 | A | C |
| ATOM | 1598 | NE | ARG | A | 516 | 16.507 | 9.747 | 31.733 | 1.00 | 15.87 | | A | N |
| ANISOU | 1598 | NE | ARG | A | 516 | 2097 | 1890 | 2040 | 13 | 34 | −130 | A | N |
| ATOM | 1600 | CZ | ARG | A | 516 | 15.551 | 9.616 | 30.834 | 1.00 | 15.53 | | A | C |
| ANISOU | 1600 | CZ | ARG | A | 516 | 1988 | 1846 | 2064 | 22 | 38 | 36 | A | C |
| ATOM | 1601 | NH1 | ARG | A | 516 | 15.789 | 9.858 | 29.545 | 1.00 | 15.41 | | A | N |
| ANISOU | 1601 | NH1 | ARG | A | 516 | 1819 | 2016 | 2019 | −58 | 1 | −112 | A | N |
| ATOM | 1604 | NH2 | ARG | A | 516 | 14.335 | 9.234 | 31.223 | 1.00 | 15.36 | | A | N |
| ANISOU | 1604 | NH2 | ARG | A | 516 | 1989 | 1737 | 2107 | 84 | 31 | 40 | A | N |
| ATOM | 1607 | C | ARG | A | 516 | 20.420 | 6.252 | 32.487 | 1.00 | 13.71 | | A | C |
| ANISOU | 1607 | C | ARG | A | 516 | 1732 | 1770 | 1707 | −29 | −61 | −67 | A | C |
| ATOM | 1608 | O | ARG | A | 516 | 21.145 | 6.580 | 33.441 | 1.00 | 13.82 | | A | O |
| ANISOU | 1608 | O | ARG | A | 516 | 1799 | 1888 | 1562 | −5 | −153 | −70 | A | O |
| ATOM | 1609 | N | ASN | A | 517 | 19.684 | 5.140 | 32.457 | 1.00 | 13.59 | | A | N |
| ANISOU | 1609 | N | ASN | A | 517 | 1662 | 1797 | 1703 | −32 | −74 | −45 | A | N |
| ATOM | 1611 | CA | ASN | A | 517 | 19.450 | 4.318 | 33.636 | 1.00 | 13.42 | | A | C |
| ANISOU | 1611 | CA | ASN | A | 517 | 1645 | 1743 | 1709 | −62 | −51 | −50 | A | C |
| ATOM | 1613 | CB | ASN | A | 517 | 17.944 | 4.137 | 33.843 | 1.00 | 13.51 | | A | C |
| ANISOU | 1613 | CB | ASN | A | 517 | 1669 | 1766 | 1697 | −8 | −46 | −16 | A | C |
| ATOM | 1616 | CG | ASN | A | 517 | 17.228 | 5.464 | 34.078 | 1.00 | 13.53 | | A | C |
| ANISOU | 1616 | CG | ASN | A | 517 | 1649 | 1722 | 1768 | 7 | −13 | −95 | A | C |
| ATOM | 1617 | OD1 | ASN | A | 517 | 16.266 | 5.815 | 33.395 | 1.00 | 14.70 | | A | O |
| ANISOU | 1617 | OD1 | ASN | A | 517 | 1848 | 2065 | 1669 | −39 | 26 | −57 | A | O |
| ATOM | 1618 | ND2 | ASN | A | 517 | 17.701 | 6.207 | 35.048 | 1.00 | 15.63 | | A | N |
| ANISOU | 1618 | ND2 | ASN | A | 517 | 1928 | 2162 | 1847 | 76 | −91 | −248 | A | N |
| ATOM | 1621 | C | ASN | A | 517 | 20.134 | 2.969 | 33.559 | 1.00 | 13.65 | | A | C |
| ANISOU | 1621 | C | ASN | A | 517 | 1641 | 1781 | 1764 | −65 | −49 | −25 | A | C |
| ATOM | 1622 | O | ASN | A | 517 | 19.929 | 2.120 | 34.425 | 1.00 | 13.34 | | A | O |
| ANISOU | 1622 | O | ASN | A | 517 | 1580 | 1856 | 1631 | −126 | −109 | −60 | A | O |
| ATOM | 1623 | N | LYS | A | 518 | 20.985 | 2.790 | 32.547 | 1.00 | 13.68 | | A | N |
| ANISOU | 1623 | N | LYS | A | 518 | 1735 | 1816 | 1644 | −95 | −36 | −67 | A | N |
| ATOM | 1625 | CA | LYS | A | 518 | 21.658 | 1.521 | 32.300 | 1.00 | 15.53 | | A | C |
| ANISOU | 1625 | CA | LYS | A | 518 | 1950 | 2007 | 1942 | 0 | −6 | 15 | A | C |
| ATOM | 1627 | CB | LYS | A | 518 | 22.707 | 1.694 | 31.184 | 1.00 | 16.30 | | A | C |
| ANISOU | 1627 | CB | LYS | A | 518 | 2067 | 2115 | 2008 | −32 | 48 | 9 | A | C |
| ATOM | 1630 | CG | LYS | A | 518 | 23.666 | 0.526 | 31.025 | 1.00 | 18.24 | | A | C |
| ANISOU | 1630 | CG | LYS | A | 518 | 2292 | 2325 | 2311 | 53 | −29 | −6 | A | C |
| ATOM | 1633 | CD | LYS | A | 518 | 24.704 | 0.806 | 29.928 | 1.00 | 21.68 | | A | C |
| ANISOU | 1633 | CD | LYS | A | 518 | 2713 | 2861 | 2660 | −51 | 70 | 36 | A | C |
| ATOM | 1636 | CE | LYS | A | 518 | 25.507 | −0.446 | 29.607 | 1.00 | 24.34 | | A | C |
| ANISOU | 1636 | CE | LYS | A | 518 | 3092 | 3036 | 3117 | 7 | 11 | −38 | A | C |
| ATOM | 1639 | NZ | LYS | A | 518 | 26.330 | −0.847 | 30.773 | 1.00 | 26.68 | | A | N |
| ANISOU | 1639 | NZ | LYS | A | 518 | 3409 | 3385 | 3341 | 2 | −86 | 44 | A | N |
| ATOM | 1643 | C | LYS | A | 518 | 22.308 | 0.908 | 33.526 | 1.00 | 15.71 | | A | C |
| ANISOU | 1643 | C | LYS | A | 518 | 1935 | 2014 | 2017 | 21 | −9 | 8 | A | C |
| ATOM | 1644 | O | LYS | A | 518 | 22.219 | −0.291 | 33.745 | 1.00 | 16.81 | | A | O |
| ANISOU | 1644 | O | LYS | A | 518 | 2136 | 2100 | 2149 | 26 | −2 | 34 | A | O |
| ATOM | 1645 | N | ASN | A | 519 | 22.957 | 1.727 | 34.327 | 1.00 | 15.57 | | A | N |
| ANISOU | 1645 | N | ASN | A | 519 | 1971 | 1985 | 1957 | 37 | 30 | 11 | A | N |
| ATOM | 1647 | CA | ASN | A | 519 | 23.700 | 1.193 | 35.464 | 1.00 | 16.90 | | A | C |
| ANISOU | 1647 | CA | ASN | A | 519 | 2124 | 2158 | 2137 | 0 | −26 | 17 | A | C |
| ATOM | 1649 | CB | ASN | A | 519 | 24.695 | 2.234 | 35.957 | 1.00 | 16.82 | | A | C |
| ANISOU | 1649 | CB | ASN | A | 519 | 2132 | 2154 | 2105 | 36 | −89 | −12 | A | C |
| ATOM | 1652 | CG | ASN | A | 519 | 25.788 | 2.499 | 34.936 | 1.00 | 19.91 | | A | C |
| ANISOU | 1652 | CG | ASN | A | 519 | 2455 | 2632 | 2477 | −38 | 8 | 4 | A | C |
| ATOM | 1653 | OD1 | ASN | A | 519 | 26.203 | 1.598 | 34.193 | 1.00 | 24.12 | | A | O |
| ANISOU | 1653 | OD1 | ASN | A | 519 | 3019 | 3078 | 3068 | 50 | 74 | −56 | A | O |
| ATOM | 1654 | ND2 | ASN | A | 519 | 26.259 | 3.734 | 34.886 | 1.00 | 24.29 | | A | N |
| ANISOU | 1654 | ND2 | ASN | A | 519 | 3097 | 2891 | 3238 | −78 | 155 | 69 | A | N |
| ATOM | 1657 | C | ASN | A | 519 | 22.830 | 0.638 | 36.587 | 1.00 | 17.17 | | A | C |
| ANISOU | 1657 | C | ASN | A | 519 | 2189 | 2211 | 2124 | 22 | −13 | 23 | A | C |
| ATOM | 1658 | O | ASN | A | 519 | 23.342 | −0.091 | 37.436 | 1.00 | 18.51 | | A | O |
| ANISOU | 1658 | O | ASN | A | 519 | 2373 | 2416 | 2243 | 16 | −31 | 109 | A | O |
| ATOM | 1659 | N | SER | A | 520 | 21.537 | 0.961 | 36.596 | 1.00 | 17.98 | | A | N |
| ANISOU | 1659 | N | SER | A | 520 | 2283 | 2363 | 2183 | −37 | −51 | 29 | A | N |
| ATOM | 1661 | CA | SER | A | 520 | 20.621 | 0.467 | 37.629 | 1.00 | 18.20 | | A | C |
| ANISOU | 1661 | CA | SER | A | 520 | 2303 | 2373 | 2238 | −52 | 1 | −13 | A | C |
| ATOM | 1663 | CB | SER | A | 520 | 19.959 | 1.652 | 38.321 | 1.00 | 18.86 | | A | C |
| ANISOU | 1663 | CB | SER | A | 520 | 2370 | 2426 | 2366 | −36 | 50 | −19 | A | C |
| ATOM | 1666 | OG | SER | A | 520 | 20.964 | 2.510 | 38.840 | 1.00 | 20.63 | | A | O |
| ANISOU | 1666 | OG | SER | A | 520 | 2612 | 2716 | 2511 | −84 | 43 | −79 | A | O |
| ATOM | 1668 | C | SER | A | 520 | 19.556 | −0.510 | 37.130 | 1.00 | 18.21 | | A | C |
| ANISOU | 1668 | C | SER | A | 520 | 2337 | 2371 | 2209 | −69 | 34 | −4 | A | C |
| ATOM | 1669 | O | SER | A | 520 | 18.767 | −1.046 | 37.904 | 1.00 | 19.67 | | A | O |
| ANISOU | 1669 | O | SER | A | 520 | 2603 | 2633 | 2238 | −134 | 118 | 16 | A | O |
| ATOM | 1670 | N | LEU | A | 521 | 19.538 | −0.766 | 35.840 | 1.00 | 16.22 | | A | N |
| ANISOU | 1670 | N | LEU | A | 521 | 2042 | 2150 | 1970 | −36 | 7 | 25 | A | N |
| ATOM | 1672 | CA | LEU | A | 521 | 18.520 | −1.634 | 35.276 | 1.00 | 15.83 | | A | C |
| ANISOU | 1672 | CA | LEU | A | 521 | 1989 | 2055 | 1969 | −15 | 28 | 9 | A | C |
| ATOM | 1674 | CB | LEU | A | 521 | 18.287 | −1.262 | 33.813 | 1.00 | 15.16 | | A | C |
| ANISOU | 1674 | CB | LEU | A | 521 | 1924 | 1969 | 1865 | −43 | −38 | −18 | A | C |
| ATOM | 1677 | CG | LEU | A | 521 | 17.445 | −0.003 | 33.626 | 1.00 | 15.36 | | A | C |
| ANISOU | 1677 | CG | LEU | A | 521 | 1994 | 1996 | 1846 | −3 | 21 | 43 | A | C |
| ATOM | 1679 | CD1 | LEU | A | 521 | 17.606 | 0.581 | 32.220 | 1.00 | 14.43 | | A | C |
| ANISOU | 1679 | CD1 | LEU | A | 521 | 1797 | 1990 | 1692 | −96 | −36 | −12 | A | C |
| ATOM | 1683 | CD2 | LEU | A | 521 | 16.004 | −0.323 | 33.878 | 1.00 | 17.25 | | A | C |
| ANISOU | 1683 | CD2 | LEU | A | 521 | 2118 | 2241 | 2194 | −22 | 19 | 63 | A | C |
| ATOM | 1687 | C | LEU | A | 521 | 18.896 | −3.099 | 35.387 | 1.00 | 15.45 | | A | C |
| ANISOU | 1687 | C | LEU | A | 521 | 1923 | 2011 | 1935 | −10 | 10 | −3 | A | C |
| ATOM | 1688 | O | LEU | A | 521 | 20.030 | −3.478 | 35.084 | 1.00 | 17.68 | | A | O |
| ANISOU | 1688 | O | LEU | A | 521 | 2097 | 2275 | 2345 | 98 | 146 | 45 | A | O |
| ATOM | 1689 | N | LYS | A | 522 | 17.956 | −3.913 | 35.848 | 1.00 | 14.61 | | A | N |
| ANISOU | 1689 | N | LYS | A | 522 | 1851 | 1934 | 1765 | −4 | 14 | −53 | A | N |
| ATOM | 1691 | CA | LYS | A | 522 | 18.112 | −5.371 | 35.897 | 1.00 | 14.30 | | A | C |
| ANISOU | 1691 | CA | LYS | A | 522 | 1788 | 1847 | 1797 | −21 | −6 | −12 | A | C |
| ATOM | 1693 | CB | LYS | A | 522 | 17.216 | −5.973 | 36.987 | 1.00 | 14.87 | | A | C |
| ANISOU | 1693 | CB | LYS | A | 522 | 1921 | 1886 | 1840 | 1 | 12 | 10 | A | C |
| ATOM | 1696 | CG | LYS | A | 522 | 17.466 | −5.498 | 38.395 | 1.00 | 17.87 | | A | C |
| ANISOU | 1696 | CG | LYS | A | 522 | 2325 | 2328 | 2137 | 57 | −25 | −72 | A | C |
| ATOM | 1699 | CD | LYS | A | 522 | 16.531 | −6.214 | 39.377 | 1.00 | 21.73 | | A | C |
| ANISOU | 1699 | CD | LYS | A | 522 | 2777 | 2767 | 2710 | −86 | 49 | 50 | A | C |
| ATOM | 1702 | CE | LYS | A | 522 | 15.213 | −5.479 | 39.565 | 1.00 | 24.11 | | A | C |
| ANISOU | 1702 | CE | LYS | A | 522 | 3008 | 3065 | 3088 | 26 | 7 | 5 | A | C |
| ATOM | 1705 | NZ | LYS | A | 522 | 14.798 | −5.470 | 41.004 | 1.00 | 26.70 | | A | N |
| ANISOU | 1705 | NZ | LYS | A | 522 | 3500 | 3390 | 3253 | −35 | 49 | 9 | A | N |
| ATOM | 1709 | C | LYS | A | 522 | 17.675 | −6.006 | 34.568 | 1.00 | 13.14 | | A | C |
| ANISOU | 1709 | C | LYS | A | 522 | 1638 | 1692 | 1662 | −19 | 11 | 17 | A | C |
| ATOM | 1710 | O | LYS | A | 522 | 16.778 | −5.503 | 33.902 | 1.00 | 12.43 | | A | O |
| ANISOU | 1710 | O | LYS | A | 522 | 1632 | 1546 | 1543 | −120 | 15 | 54 | A | O |
| ATOM | 1711 | N | VAL | A | 523 | 18.255 | −7.155 | 34.230 | 1.00 | 12.02 | | A | N |
| ANISOU | 1711 | N | VAL | A | 523 | 1542 | 1542 | 1479 | −43 | −2 | −9 | A | N |
| ATOM | 1713 | CA | VAL | A | 523 | 17.857 | −7.882 | 33.025 | 1.00 | 12.05 | | A | C |
| ANISOU | 1713 | CA | VAL | A | 523 | 1557 | 1579 | 1441 | −36 | −11 | 33 | A | C |
| ATOM | 1715 | CB | VAL | A | 523 | 18.715 | −9.145 | 32.800 | 1.00 | 12.93 | | A | C |
| ANISOU | 1715 | CB | VAL | A | 523 | 1625 | 1676 | 1609 | −8 | 16 | −1 | A | C |
| ATOM | 1717 | CG1 | VAL | A | 523 | 18.210 | −9.948 | 31.626 | 1.00 | 13.27 | | A | C |
| ANISOU | 1717 | CG1 | VAL | A | 523 | 1660 | 1675 | 1706 | −11 | 67 | 17 | A | C |
| ATOM | 1721 | GG2 | VAL | A | 523 | 20.188 | −8.786 | 32.588 | 1.00 | 14.25 | | A | C |
| ANISOU | 1721 | CG2 | VAL | A | 523 | 1800 | 1944 | 1669 | −68 | 48 | 12 | A | C |
| ATOM | 1725 | C | VAL | A | 523 | 16.373 | −8.272 | 33.117 | 1.00 | 11.75 | | A | C |
| ANISOU | 1725 | C | VAL | A | 523 | 1499 | 1561 | 1404 | −28 | −32 | 33 | A | C |
| ATOM | 1726 | O | VAL | A | 523 | 15.664 | −8.247 | 32.143 | 1.00 | 10.50 | | A | O |
| ANISOU | 1726 | O | VAL | A | 523 | 1362 | 1386 | 1239 | −81 | −108 | 202 | A | O |
| ATOM | 1727 | N | LEU | A | 524 | 15.911 | −8.624 | 34.306 | 1.00 | 12.33 | | A | N |
| ANISOU | 1727 | N | LEU | A | 524 | 1565 | 1612 | 1509 | −56 | −42 | 93 | A | N |
| ATOM | 1729 | CA | LEU | A | 524 | 14.514 | −8.914 | 34.560 | 1.00 | 13.41 | | A | C |
| ANISOU | 1729 | CA | LEU | A | 524 | 1652 | 1798 | 1645 | −2 | −14 | 69 | A | C |
| ATOM | 1731 | CB | LEU | A | 524 | 14.344 | −8.986 | 36.094 | 1.00 | 15.16 | | A | C |
| ANISOU | 1731 | CB | LEU | A | 524 | 1890 | 2012 | 1855 | 47 | −50 | 179 | A | C |
| ATOM | 1734 | CG | LEU | A | 524 | 13.061 | −9.363 | 36.763 | 1.00 | 20.08 | | A | C |
| ANISOU | 1734 | CG | LEU | A | 524 | 2450 | 2684 | 2495 | −80 | 45 | 48 | A | C |
| ATOM | 1736 | CD1 | LEU | A | 524 | 12.446 | −10.522 | 36.037 | 1.00 | 21.92 | | A | C |
| ANISOU | 1736 | CD1 | LEU | A | 524 | 2859 | 2718 | 2749 | −31 | −32 | −41 | A | C |
| ATOM | 1740 | CD2 | LEU | A | 524 | 13.434 | −9.741 | 38.204 | 1.00 | 21.61 | | A | C |
| ANISOU | 1740 | CD2 | LEU | A | 524 | 2929 | 2684 | 2597 | −69 | 0 | 58 | A | C |
| ATOM | 1744 | C | LEU | A | 524 | 13.587 | −7.840 | 33.995 | 1.00 | 12.32 | | A | C |
| ANISOU | 1744 | C | LEU | A | 524 | 1513 | 1658 | 1510 | −52 | −37 | 49 | A | C |
| ATOM | 1745 | O | LEU | A | 524 | 12.552 | −8.131 | 33.384 | 1.00 | 11.88 | | A | O |
| ANISOU | 1745 | O | LEU | A | 524 | 1415 | 1637 | 1463 | −181 | −42 | 103 | A | O |
| ATOM | 1746 | N | THR | A | 525 | 13.959 | −6.590 | 34.218 | 1.00 | 11.05 | | A | N |
| ANISOU | 1746 | N | THR | A | 525 | 1336 | 1613 | 1247 | −93 | −20 | 59 | A | N |
| ATOM | 1748 | CA | THR | A | 525 | 13.190 | −5.447 | 33.789 | 1.00 | 10.85 | | A | C |
| ANISOU | 1748 | CA | THR | A | 525 | 1328 | 1497 | 1296 | −65 | 77 | 0 | A | C |
| ATOM | 1750 | CB | THR | A | 525 | 13.745 | −4.215 | 34.469 | 1.00 | 11.61 | | A | C |
| ANISOU | 1750 | CB | THR | A | 525 | 1393 | 1707 | 1312 | −99 | 41 | −17 | A | C |
| ATOM | 1752 | OG1 | THR | A | 525 | 13.715 | −4.414 | 35.900 | 1.00 | 12.91 | | A | O |
| ANISOU | 1752 | OG1 | THR | A | 525 | 1548 | 2104 | 1250 | −74 | 20 | −137 | A | O |
| ATOM | 1754 | CG2 | THR | A | 525 | 12.913 | −2.990 | 34.189 | 1.00 | 12.19 | | A | C |
| ANISOU | 1754 | CG2 | THR | A | 525 | 1507 | 1718 | 1407 | −66 | 33 | −104 | A | C |
| ATOM | 1758 | C | THR | A | 525 | 13.205 | −5.257 | 32.273 | 1.00 | 10.45 | | A | C |
| ANISOU | 1758 | C | THR | A | 525 | 1242 | 1438 | 1289 | −72 | 6 | 23 | A | C |
| ATOM | 1759 | O | THR | A | 525 | 12.219 | −4.870 | 31.680 | 1.00 | 10.57 | | A | O |
| ANISOU | 1759 | O | THR | A | 525 | 1167 | 1388 | 1460 | −115 | −24 | 34 | A | O |
| ATOM | 1760 | N | LEU | A | 526 | 14.334 | −5.549 | 31.657 | 1.00 | 9.36 | | A | N |
| ANISOU | 1760 | N | LEU | A | 526 | 1174 | 1259 | 1122 | −35 | −7 | −12 | A | C |
| ATOM | 1762 | CA | LEU | A | 526 | 14.460 | −5.438 | 30.215 | 1.00 | 9.41 | | A | C |
| ANISOU | 1762 | CA | LEU | A | 526 | 1225 | 1222 | 1128 | −39 | −2 | 43 | A | C |
| ATOM | 1764 | CB | LEU | A | 526 | 15.918 | −5.623 | 29.831 | 1.00 | 9.14 | | A | C |
| ANISOU | 1764 | CB | LEU | A | 526 | 1189 | 1238 | 1044 | −58 | 36 | 17 | A | C |
| ATOM | 1767 | CG | LEU | A | 526 | 16.904 | −4.618 | 30.404 | 1.00 | 9.74 | | A | C |
| ANISOU | 1767 | CG | LEU | A | 526 | 1262 | 1281 | 1155 | 10 | −89 | −20 | A | C |
| ATOM | 1769 | CD1 | LEU | A | 526 | 18.322 | −4.916 | 29.952 | 1.00 | 10.71 | | A | C |
| ANISOU | 1769 | CD1 | LEU | A | 526 | 1380 | 1362 | 1325 | −103 | 18 | −71 | A | C |
| ATOM | 1773 | CD2 | LEU | A | 526 | 16.492 | −3.182 | 30.095 | 1.00 | 10.98 | | A | C |
| ANISOU | 1773 | CD2 | LEU | A | 526 | 1543 | 1304 | 1324 | −108 | −23 | −22 | A | C |
| ATOM | 1777 | C | LEU | A | 526 | 13.582 | −6.491 | 29.536 | 1.00 | 9.09 | | A | C |
| ANISOU | 1777 | C | LEU | A | 526 | 1242 | 1185 | 1026 | −16 | 15 | −1 | A | C |
| ATOM | 1778 | O | LEU | A | 526 | 12.988 | −6.233 | 28.493 | 1.00 | 8.30 | | A | O |
| ANISOU | 1778 | O | LEU | A | 526 | 1217 | 878 | 1055 | 41 | 20 | 90 | A | O |
| ATOM | 1779 | N | VAL | A | 527 | 13.504 | −7.676 | 30.145 | 1.00 | 8.84 | | A | N |
| ANISOU | 1779 | N | VAL | A | 527 | 1247 | 1178 | 932 | −52 | −38 | −14 | A | N |
| ATOM | 1781 | CA | VAL | A | 527 | 12.645 | −8.743 | 29.661 | 1.00 | 9.54 | | A | C |
| ANISOU | 1781 | CA | VAL | A | 527 | 1243 | 1166 | 1215 | −61 | 12 | 34 | A | C |
| ATOM | 1783 | CB | VAL | A | 527 | 12.969 | −10.092 | 30.305 | 1.00 | 9.86 | | A | C |
| ANISOU | 1783 | CB | VAL | A | 527 | 1269 | 1253 | 1224 | 13 | −16 | 10 | A | C |
| ATOM | 1785 | CG1 | VAL | A | 527 | 11.984 | −11.135 | 29.857 | 1.00 | 11.97 | | A | C |
| ANISOU | 1785 | CG1 | VAL | A | 527 | 1439 | 1479 | 1628 | −88 | 7 | 109 | A | C |
| ATOM | 1789 | CG2 | VAL | A | 527 | 14.369 | −10.514 | 29.931 | 1.00 | 9.87 | | A | C |
| ANISOU | 1789 | CG2 | VAL | A | 527 | 1239 | 1058 | 1451 | −49 | 2 | −76 | A | C |
| ATOM | 1793 | C | VAL | A | 527 | 11.175 | −8.343 | 29.859 | 1.00 | 9.36 | | A | C |
| ANISOU | 1793 | C | VAL | A | 527 | 1209 | 1152 | 1193 | −27 | −10 | −29 | A | C |
| ATOM | 1794 | O | VAL | A | 527 | 10.343 | −8.566 | 28.983 | 1.00 | 9.72 | | A | O |
| ANISOU | 1794 | O | VAL | A | 527 | 1239 | 1278 | 1175 | O | −62 | −37 | A | O |
| ATOM | 1795 | N | LEU | A | 528 | 10.860 | −7.725 | 30.994 | 1.00 | 10.29 | | A | N |
| ANISOU | 1795 | N | LEU | A | 528 | 1267 | 1299 | 1344 | −42 | 29 | −48 | A | N |
| ATOM | 1797 | CA | LEU | A | 528 | 9.489 | −7.280 | 31.261 | 1.00 | 10.35 | | A | C |
| ANISOU | 1797 | CA | LEU | A | 528 | 1306 | 1331 | 1293 | −41 | 0 | 35 | A | C |
| ATOM | 1799 | CB | LEU | A | 528 | 9.389 | −6.614 | 32.632 | 1.00 | 10.74 | | A | C |
| ANISOU | 1799 | CB | LEU | A | 528 | 1459 | 1244 | 1375 | −78 | 0 | 10 | A | C |
| ATOM | 1802 | CG | LEU | A | 528 | 8.064 | −5.937 | 32.926 | 1.00 | 12.29 | | A | C |
| ANISOU | 1802 | CG | LEU | A | 528 | 1533 | 1539 | 1595 | −63 | −13 | 20 | A | C |
| ATOM | 1804 | CD1 | LEU | A | 528 | 6.941 | −6.975 | 33.017 | 1.00 | 13.69 | | A | C |
| ANISOU | 1804 | CD1 | LEU | A | 528 | 1574 | 1770 | 1857 | −117 | 60 | 20 | A | C |
| ATOM | 1808 | CD2 | LEU | A | 528 | 8.153 | −5.126 | 34.181 | 1.00 | 13.38 | | A | C |
| ANISOU | 1808 | CD2 | LEU | A | 528 | 1639 | 1734 | 1709 | 24 | 52 | −63 | A | C |
| ATOM | 1812 | C | LEU | A | 528 | 9.034 | −6.300 | 30.181 | 1.00 | 9.90 | | A | C |
| ANISOU | 1812 | C | LEU | A | 528 | 1244 | 1162 | 1354 | −47 | −23 | 41 | A | C |
| ATOM | 1813 | O | LEU | A | 528 | 7.924 | −6.413 | 29.648 | 1.00 | 10.14 | | A | O |
| ANISOU | 1813 | O | LEU | A | 528 | 1302 | 1282 | 1268 | −70 | −81 | 0 | A | O |
| ATOM | 1814 | N | TYR | A | 529 | 9.889 | −5.317 | 29.864 | 1.00 | 9.63 | | A | N |
| ANISOU | 1814 | N | TYR | A | 529 | 1073 | 1283 | 1301 | −114 | −29 | 47 | A | N |
| ATOM | 1816 | CA | TYR | A | 529 | 9.529 | −4.351 | 28.843 | 1.00 | 9.14 | | A | C |
| ANISOU | 1816 | CA | TYR | A | 529 | 1094 | 1143 | 1236 | −17 | −8 | −11 | A | C |
| ATOM | 1818 | CB | TYR | A | 529 | 10.588 | −3.258 | 28.672 | 1.00 | 9.37 | | A | C |
| ANISOU | 1818 | CB | TYR | A | 529 | 1153 | 1152 | 1252 | −53 | −18 | 28 | A | C |
| ATOM | 1821 | CG | TYR | A | 529 | 10.825 | −2.388 | 29.893 | 1.00 | 10.76 | | A | C |
| ANISOU | 1821 | CG | TYR | A | 529 | 1408 | 1370 | 1309 | −3 | 33 | −15 | A | C |
| ATOM | 1822 | CD1 | TYR | A | 529 | 9.882 | −2.244 | 30.912 | 1.00 | 11.31 | | A | C |
| ANISOU | 1822 | CD1 | TYR | A | 529 | 1491 | 1376 | 1427 | −184 | 2 | −128 | A | C |
| ATOM | 1824 | CE1 | TYR | A | 529 | 10.146 | −1.438 | 32.006 | 1.00 | 11.43 | | A | C |
| ANISOU | 1824 | CE1 | TYR | A | 529 | 1582 | 1461 | 1297 | −78 | 31 | −171 | A | C |
| ATOM | 1826 | CZ | TYR | A | 529 | 11.352 | −0.759 | 32.072 | 1.00 | 12.02 | | A | C |
| ANISOU | 1826 | CZ | TYR | A | 529 | 1606 | 1411 | 1550 | −45 | 101 | −145 | A | C |
| ATOM | 1827 | OH | TYR | A | 529 | 11.660 | 0.055 | 33.154 | 1.00 | 14.10 | | A | O |
| ANISOU | 1827 | OH | TYR | A | 529 | 1938 | 1820 | 1597 | −4 | 68 | −324 | A | O |
| ATOM | 1829 | CE2 | TYR | A | 529 | 12.282 | −0.880 | 31.084 | 1.00 | 12.26 | | A | C |
| ANISOU | 1829 | CE2 | TYR | A | 529 | 1666 | 1490 | 1502 | −114 | 47 | 16 | A | C |
| ATOM | 1831 | CD2 | TYR | A | 529 | 12.018 | −1.676 | 30.006 | 1.00 | 12.33 | | A | C |
| ANISOU | 1831 | CD2 | TYR | A | 529 | 1595 | 1567 | 1523 | −118 | 67 | −77 | A | C |
| ATOM | 1833 | C | TYR | A | 529 | 9.269 | −5.030 | 27.508 | 1.00 | 8.97 | | A | C |
| ANISOU | 1833 | C | TYR | A | 529 | 1106 | 1094 | 1206 | −32 | −18 | −16 | A | C |
| ATOM | 1834 | O | TYR | A | 529 | 8.302 | −4.695 | 26.809 | 1.00 | 9.43 | | A | O |
| ANISOU | 1834 | O | TYR | A | 529 | 1072 | 1210 | 1300 | −40 | 41 | −33 | A | O |
| ATOM | 1835 | N | SER | A | 530 | 10.114 | −5.992 | 27.154 | 1.00 | 8.94 | | A | N |
| ANISOU | 1835 | N | SER | A | 530 | 1036 | 1132 | 1229 | −61 | −24 | −9 | A | N |
| ATOM | 1837 | CA | SER | A | 530 | 9.947 | −6.739 | 25.904 | 1.00 | 8.84 | | A | C |
| ANISOU | 1837 | CA | SER | A | 530 | 1083 | 1116 | 1159 | −30 | 29 | 37 | A | C |
| ATOM | 1839 | CB | SER | A | 530 | 11.079 | −7.742 | 25.714 | 1.00 | 9.23 | | A | C |
| ANISOU | 1839 | CB | SER | A | 530 | 1075 | 1115 | 1316 | −53 | 14 | 14 | A | C |
| ATOM | 1842 | OG | SER | A | 530 | 12.340 | −7.132 | 25.627 | 1.00 | 10.06 | | A | O |
| ANISOU | 1842 | OG | SER | A | 530 | 1121 | 1154 | 1545 | −63 | 30 | 189 | A | O |
| ATOM | 1844 | C | SER | A | 530 | 8.612 | −7.473 | 25.888 | 1.00 | 8.46 | | A | C |
| ANISOU | 1844 | C | SER | A | 530 | 1026 | 1096 | 1092 | 17 | 16 | 2 | A | C |
| ATOM | 1845 | O | SER | A | 530 | 7.913 | −7.487 | 24.889 | 1.00 | 8.24 | | A | O |
| ANISOU | 1845 | O | SER | A | 530 | 963 | 1078 | 1089 | 132 | 6 | 83 | A | O |
| ATOM | 1846 | N | LEU | A | 531 | 8.269 | −8.109 | 27.000 | 1.00 | 9.01 | | A | N |
| ANISOU | 1846 | N | LEU | A | 531 | 1117 | 1139 | 1165 | −15 | −47 | 74 | A | N |
| ATOM | 1848 | CA | LEU | A | 531 | 7.001 | −8.827 | 27.133 | 1.00 | 8.68 | | A | C |
| ANISOU | 1848 | CA | LEU | A | 531 | 1116 | 1055 | 1124 | −34 | 27 | −56 | A | C |
| ATOM | 1850 | CB | LEU | A | 531 | 6.971 | −9.559 | 28.464 | 1.00 | 9.19 | | A | C |
| ANISOU | 1850 | CB | LEU | A | 531 | 1163 | 1088 | 1240 | −14 | −28 | −50 | A | C |
| ATOM | 1853 | CG | LEU | A | 531 | 5.685 | −10.272 | 28.860 | 1.00 | 8.73 | | A | C |
| ANISOU | 1853 | CG | LEU | A | 531 | 1172 | 1052 | 1091 | 43 | 39 | −44 | A | C |
| ATOM | 1855 | CD1 | LEU | A | 531 | 5.290 | −11.366 | 27.860 | 1.00 | 9.70 | | A | C |
| ANISOU | 1855 | CD1 | LEU | A | 531 | 1345 | 1059 | 1281 | −62 | 17 | −25 | A | C |
| ATOM | 1859 | CD2 | LEU | A | 531 | 5.954 | −10.879 | 30.255 | 1.00 | 10.76 | | A | C |
| ANISOU | 1859 | CD2 | LEU | A | 531 | 1301 | 1546 | 1241 | −162 | 132 | 118 | A | C |
| ATOM | 1863 | C | LEU | A | 531 | 5.796 | −7.900 | 27.005 | 1.00 | 9.13 | | A | C |
| ANISOU | 1863 | C | LEU | A | 531 | 1146 | 1127 | 1193 | −21 | −18 | −57 | A | C |
| ATOM | 1864 | O | LEU | A | 531 | 4.810 | −8.224 | 26.348 | 1.00 | 8.80 | | A | O |
| ANISOU | 1864 | O | LEU | A | 531 | 1021 | 1070 | 1251 | −55 | 1 | −6 | A | O |
| ATOM | 1865 | N | GLN | A | 532 | 5.867 | −6.732 | 27.636 | 1.00 | 9.21 | | A | N |
| ANISOU | 1865 | N | GLN | A | 532 | 1136 | 1083 | 1278 | −66 | −44 | −34 | A | N |
| ATOM | 1867 | CA | GLN | A | 532 | 4.768 | −5.769 | 27.593 | 1.00 | 9.35 | | A | C |
| ANISOU | 1867 | CA | GLN | A | 532 | 1181 | 1148 | 1223 | −29 | −33 | −46 | A | C |
| ATOM | 1869 | CB | GLN | A | 532 | 5.110 | −4.583 | 28.474 | 1.00 | 9.02 | | A | C |
| ANISOU | 1869 | CB | GLN | A | 532 | 1057 | 1113 | 1255 | 99 | 21 | −149 | A | C |
| ATOM | 1872 | CG | GLN | A | 532 | 5.060 | −4.948 | 29.954 | 1.00 | 9.32 | | A | C |
| ANISOU | 1872 | CG | GLN | A | 532 | 1099 | 1136 | 1305 | 6 | −15 | −33 | A | C |
| ATOM | 1875 | CD | GLN | A | 532 | 5.341 | −3.774 | 30.882 | 1.00 | 9.94 | | A | C |
| ANISOU | 1875 | CD | GLN | A | 532 | 1192 | 1298 | 1287 | −43 | −49 | 15 | A | C |
| ATOM | 1876 | OE1 | GLN | A | 532 | 5.859 | −2.743 | 30.446 | 1.00 | 10.56 | | A | O |
| ANISOU | 1876 | OE1 | GLN | A | 532 | 1584 | 1343 | 1084 | −169 | 155 | −60 | A | O |
| ATOM | 1877 | NE2 | GLN | A | 532 | 4.988 | −3.930 | 32.163 | 1.00 | 10.28 | | A | N |
| ANISOU | 1877 | NE2 | GLN | A | 532 | 1277 | 1497 | 1130 | 136 | −26 | −131 | A | N |
| ATOM | 1880 | C | GLN | A | 532 | 4.512 | −5.319 | 26.166 | 1.00 | 8.73 | | A | C |
| ANISOU | 1880 | C | GLN | A | 532 | 1089 | 1053 | 1175 | −12 | 0 | −32 | A | C |
| ATOM | 1881 | O | GLN | A | 532 | 3.382 | −5.302 | 25.727 | 1.00 | 9.39 | | A | O |
| ANISOU | 1881 | O | GLN | A | 532 | 1118 | 1217 | 1231 | 35 | −32 | −81 | A | O |
| ATOM | 1882 | N | ILE | A | 533 | 5.572 | −4.985 | 25.435 | 1.00 | 8.53 | | A | N |
| ANISOU | 1882 | N | ILE | A | 533 | 1058 | 1056 | 1127 | −3 | −11 | −22 | A | N |
| ATOM | 1884 | CA | ILE | A | 533 | 5.432 | −4.587 | 24.031 | 1.00 | 8.51 | | A | C |
| ANISOU | 1884 | CA | ILE | A | 533 | 1073 | 1079 | 1081 | 6 | 43 | −22 | A | C |
| ATOM | 1886 | CB | ILE | A | 533 | 6.751 | −4.017 | 23.491 | 1.00 | 9.50 | | A | C |
| ANISOU | 1886 | CB | ILE | A | 533 | 1187 | 1129 | 1292 | −25 | −7 | −5 | A | C |
| ATOM | 1888 | CG1 | ILE | A | 533 | 7.157 | −2.772 | 24.301 | 1.00 | 9.86 | | A | C |
| ANISOU | 1888 | CG1 | ILE | A | 533 | 1199 | 1147 | 1398 | −86 | 20 | −48 | A | C |
| ATOM | 1891 | CD1 | ILE | A | 533 | 6.245 | −1.619 | 24.164 | 1.00 | 12.76 | | A | C |
| ANISOU | 1891 | CD1 | ILE | A | 533 | 1616 | 1463 | 1769 | −39 | −67 | 67 | A | C |
| ATOM | 1895 | CG2 | ILE | A | 533 | 6.651 | −3.765 | 21.994 | 1.00 | 10.62 | | A | C |
| ANISOU | 1895 | CG2 | ILE | A | 533 | 1420 | 1256 | 1357 | −75 | −79 | 23 | A | C |
| ATOM | 1899 | C | ILE | A | 533 | 4.908 | −5.761 | 23.197 | 1.00 | 8.60 | | A | C |
| ANISOU | 1899 | C | ILE | A | 533 | 1125 | 1061 | 1079 | −43 | −15 | 70 | A | C |
| ATOM | 1900 | O | ILE | A | 533 | 4.069 | −5.563 | 22.307 | 1.00 | 8.84 | | A | O |
| ANISOU | 1900 | O | ILE | A | 533 | 1134 | 1016 | 1208 | 55 | −227 | −5 | A | O |
| ATOM | 1901 | N | CYS | A | 534 | 5.366 | −6.971 | 23.481 | 1.00 | 8.34 | | A | N |
| ANISOU | 1901 | N | CYS | A | 534 | 1104 | 1116 | 946 | 6 | −90 | −22 | A | N |
| ATOM | 1903 | CA | CYS | A | 534 | 4.882 | −8.152 | 22.777 | 1.00 | 8.69 | | A | C |
| ANISOU | 1903 | CA | CYS | A | 534 | 1093 | 1101 | 1105 | 25 | −27 | −26 | A | C |
| ATOM | 1905 | CB | CYS | A | 534 | 5.637 | −9.377 | 23.259 | 1.00 | 8.61 | | A | C |
| ANISOU | 1905 | CB | CYS | A | 534 | 1186 | 972 | 1113 | −64 | 7 | 42 | A | C |
| ATOM | 1908 | SG | CYS | A | 534 | 5.538 | −10.759 | 22.092 | 1.00 | 9.98 | | A | S |
| ANISOU | 1908 | SG | CYS | A | 534 | 1431 | 1011 | 1350 | 32 | −138 | −122 | A | S |
| ATOM | 1909 | C | CYS | A | 534 | 3.375 | −8.345 | 22.957 | 1.00 | 8.38 | | A | C |
| ANISOU | 1909 | C | CYS | A | 534 | 1059 | 1083 | 1039 | −15 | −58 | −4 | A | C |
| ATOM | 1910 | O | CYS | A | 534 | 2.684 | −8.727 | 22.023 | 1.00 | 8.76 | | A | O |
| ANISOU | 1910 | O | CYS | A | 534 | 1180 | 1296 | 851 | −32 | −39 | −122 | A | O |
| ATOM | 1911 | N | LYS | A | 535 | 2.862 | −8.093 | 24.163 | 1.00 | 9.20 | | A | N |
| ANISOU | 1911 | N | LYS | A | 535 | 1085 | 1264 | 1144 | −23 | −30 | −78 | A | N |
| ATOM | 1913 | CA | LYS | A | 535 | 1.428 | −8.203 | 24.428 | 1.00 | 9.52 | | A | C |
| ANISOU | 1913 | CA | LYS | A | 535 | 1118 | 1292 | 1205 | 32 | 1 | −52 | A | C |
| ATOM | 1915 | CB | LYS | A | 535 | 1.142 | −8.059 | 25.924 | 1.00 | 9.70 | | A | C |
| ANISOU | 1915 | CB | LYS | A | 535 | 1074 | 1340 | 1270 | −39 | 21 | −38 | A | C |
| ATOM | 1918 | CG | LYS | A | 535 | 1.581 | −9.276 | 26.716 | 1.00 | 12.27 | | A | C |
| ANISOU | 1918 | CG | LYS | A | 535 | 1420 | 1659 | 1582 | 44 | 2 | 85 | A | C |
| ATOM | 1921 | CD | LYS | A | 535 | 1.302 | −9.083 | 28.189 | 1.00 | 15.41 | | A | C |
| ANISOU | 1921 | CD | LYS | A | 535 | 1860 | 2145 | 1849 | 6 | 22 | −50 | A | C |
| ATOM | 1924 | CE | LYS | A | 535 | 1.591 | −10.341 | 28.971 | 1.00 | 17.41 | | A | C |
| ANISOU | 1924 | CE | LYS | A | 535 | 2173 | 2286 | 2154 | −18 | 48 | 28 | A | C |
| ATOM | 1927 | NZ | LYS | A | 535 | 1.182 | −10.136 | 30.386 | 1.00 | 20.70 | | A | N |
| ANISOU | 1927 | NZ | LYS | A | 535 | 2737 | 2901 | 2225 | −8 | 166 | 51 | A | N |
| ATOM | 1931 | C | LYS | A | 535 | 0.640 | −7.181 | 23.606 | 1.00 | 8.70 | | A | C |
| ANISOU | 1931 | C | LYS | A | 535 | 1049 | 1181 | 1076 | −24 | 37 | 1 | A | C |
| ATOM | 1932 | O | LYS | A | 535 | −0.421 | −7.489 | 23.046 | 1.00 | 9.75 | | A | O |
| ANISOU | 1932 | O | LYS | A | 535 | 1211 | 1261 | 1232 | 4 | −35 | −141 | A | O |
| ATOM | 1933 | N | ALA | A | 536 | 1.147 | −5.957 | 23.504 | 1.00 | 9.18 | | A | N |
| ANISOU | 1933 | N | ALA | A | 536 | 1196 | 1172 | 1118 | 61 | −42 | −76 | A | N |
| ATOM | 1935 | CA | ALA | A | 536 | 0.544 | −4.974 | 22.601 | 1.00 | 8.93 | | A | C |
| ANISOU | 1935 | CA | ALA | A | 536 | 1079 | 1204 | 1110 | 100 | 23 | −34 | A | C |
| ATOM | 1937 | CB | ALA | A | 536 | 1.287 | −3.675 | 22.658 | 1.00 | 9.97 | | A | C |
| ANISOU | 1937 | CB | ALA | A | 536 | 1288 | 1240 | 1258 | 112 | 22 | −85 | A | C |
| ATOM | 1941 | C | ALA | A | 536 | 0.475 | −5.497 | 21.174 | 1.00 | 8.90 | | A | C |
| ANISOU | 1941 | C | ALA | A | 536 | 1051 | 1234 | 1093 | 62 | −53 | −63 | A | C |
| ATOM | 1942 | O | ALA | A | 536 | −0.541 | −5.348 | 20.486 | 1.00 | 9.18 | | A | O |
| ANISOU | 1942 | O | ALA | A | 536 | 977 | 1349 | 1159 | 187 | 9 | 36 | A | O |
| ATOM | 1943 | N | MET | A | 537 | 1.576 | −6.078 | 20.703 | 1.00 | 8.38 | | A | N |
| ANISOU | 1943 | N | MET | A | 537 | 969 | 1184 | 1029 | 69 | −70 | −27 | A | N |
| ATOM | 1945 | CA | MET | A | 537 | 1.656 | −6.598 | 19.345 | 1.00 | 9.14 | | A | C |
| ANISOU | 1945 | CA | MET | A | 537 | 1075 | 1257 | 1137 | 52 | −34 | −32 | A | C |
| ATOM | 1947 | CB | MET | A | 537 | 3.106 | −6.895 | 18.960 | 1.00 | 9.20 | | A | C |
| ANISOU | 1947 | CB | MET | A | 537 | 1148 | 1184 | 1161 | 137 | 12 | −128 | A | C |
| ATOM | 1950 | CG | MET | A | 537 | 3.944 | −5.630 | 18.797 | 1.00 | 11.09 | | A | C |
| ANISOU | 1950 | CG | MET | A | 537 | 1223 | 1582 | 1409 | −13 | 94 | −197 | A | C |
| ATOM | 1953 | SD | MET | A | 537 | 3.206 | −4.382 | 17.684 | 1.00 | 13.43 | | A | S |
| ANISOU | 1953 | SD | MET | A | 537 | 1844 | 1254 | 2003 | 62 | 403 | −32 | A | S |
| ATOM | 1954 | CE | MET | A | 537 | 2.792 | −5.416 | 16.235 | 1.00 | 14.20 | | A | C |
| ANISOU | 1954 | CE | MET | A | 537 | 1922 | 1653 | 1819 | 134 | 102 | 123 | A | C |
| ATOM | 1958 | C | MET | A | 537 | 0.755 | −7.791 | 19.132 | 1.00 | 8.69 | | A | C |
| ANISOU | 1958 | C | MET | A | 537 | 1067 | 1175 | 1057 | 69 | 4 | −23 | A | C |
| ATOM | 1959 | O | MET | A | 537 | 0.191 | −7.938 | 18.045 | 1.00 | 9.24 | | A | O |
| ANISOU | 1959 | O | MET | A | 537 | 975 | 1485 | 1050 | 118 | −55 | −9 | A | O |
| ATOM | 1960 | N | ALA | A | 538 | 0.561 | −8.635 | 20.147 | 1.00 | 9.38 | | A | N |
| ANISOU | 1960 | N | ALA | A | 538 | 1213 | 1268 | 1083 | −14 | −77 | 2 | A | N |
| ATOM | 1962 | CA | ALA | A | 538 | −0.374 | −9.741 | 20.011 | 1.00 | 8.94 | | A | C |
| ANISOU | 1962 | CA | ALA | A | 538 | 1177 | 1182 | 1038 | 5 | −33 | −35 | A | C |
| ATOM | 1964 | CB | ALA | A | 538 | −0.346 | −10.626 | 21.231 | 1.00 | 9.08 | | A | C |
| ANISOU | 1964 | CB | ALA | A | 538 | 1141 | 1074 | 1233 | 14 | 11 | 53 | A | C |
| ATOM | 1968 | C | ALA | A | 538 | −1.774 | −9.180 | 19.782 | 1.00 | 9.51 | | A | C |
| ANISOU | 1968 | C | ALA | A | 538 | 1216 | 1276 | 1121 | −11 | −1 | −55 | A | C |
| ATOM | 1969 | O | ALA | A | 538 | −2.557 | −9.735 | 18.995 | 1.00 | 9.57 | | A | O |
| ANISOU | 1969 | O | ALA | A | 538 | 1046 | 1423 | 1164 | −75 | −73 | −128 | A | O |
| ATOM | 1970 | N | TYR | A | 539 | −2.114 | −8.092 | 20.457 | 1.00 | 9.75 | | A | N |
| ANISOU | 1970 | N | TYR | A | 539 | 1179 | 1314 | 1212 | 21 | 7 | −38 | A | N |
| ATOM | 1972 | CA | TYR | A | 539 | −3.407 | −7.484 | 20.197 | 1.00 | 10.52 | | A | C |
| ANISOU | 1972 | CA | TYR | A | 539 | 1245 | 1384 | 1367 | 53 | 17 | −64 | A | C |
| ATOM | 1974 | CB | TYR | A | 539 | −3.710 | −6.383 | 21.215 | 1.00 | 11.10 | | A | C |
| ANISOU | 1974 | CB | TYR | A | 539 | 1436 | 1402 | 1380 | −26 | 9 | −83 | A | C |
| ATOM | 1977 | CG | TYR | A | 539 | −4.991 | −5.665 | 20.906 | 1.00 | 12.72 | | A | C |
| ANISOU | 1977 | CG | TYR | A | 539 | 1627 | 1769 | 1436 | 156 | 88 | −137 | A | C |
| ATOM | 1978 | CD1 | TYR | A | 539 | −6.187 | −6.360 | 20.802 | 1.00 | 15.35 | | A | C |
| ANISOU | 1978 | CD1 | TYR | A | 539 | 1903 | 1979 | 1949 | 56 | 81 | −13 | A | C |
| ATOM | 1980 | CE1 | TYR | A | 539 | −7.383 | −5.734 | 20.478 | 1.00 | 17.27 | | A | C |
| ANISOU | 1980 | CE1 | TYR | A | 539 | 2084 | 2223 | 2252 | 46 | 85 | 78 | A | C |
| ATOM | 1982 | CZ | TYR | A | 539 | −7.407 | −4.392 | 20.279 | 1.00 | 17.42 | | A | C |
| ANISOU | 1982 | CZ | TYR | A | 539 | 2158 | 2210 | 2248 | 154 | 128 | −16 | A | C |
| ATOM | 1983 | OH | TYR | A | 539 | −8.635 | −3.817 | 19.972 | 1.00 | 20.79 | | A | O |
| ANISOU | 1983 | OH | TYR | A | 539 | 2483 | 2758 | 2656 | 265 | −154 | 102 | A | O |
| ATOM | 1985 | CE2 | TYR | A | 539 | −6.236 | −3.650 | 20.387 | 1.00 | 18.14 | | A | C |
| ANISOU | 1985 | CE2 | TYR | A | 539 | 2265 | 2231 | 2395 | 131 | −31 | −7 | A | C |
| ATOM | 1987 | CD2 | TYR | A | 539 | −5.011 | −4.305 | 20.687 | 1.00 | 16.60 | | A | C |
| ANISOU | 1987 | CD2 | TYR | A | 539 | 2066 | 2069 | 2170 | 14 | 174 | 55 | A | C |
| ATOM | 1989 | C | TYR | A | 539 | −3.509 | −6.967 | 18.769 | 1.00 | 9.93 | | A | C |
| ANISOU | 1989 | C | TYR | A | 539 | 1089 | 1354 | 1330 | −21 | 5 | −61 | A | C |
| ATOM | 1990 | O | TYR | A | 539 | −4.509 | −7.190 | 18.067 | 1.00 | 10.52 | | A | O |
| ANISOU | 1990 | O | TYR | A | 539 | 989 | 1647 | 1358 | 17 | 32 | −85 | A | O |
| ATOM | 1991 | N | LEU | A | 540 | −2.499 | −6.223 | 18.310 | 1.00 | 9.16 | | A | N |
| ANISOU | 1991 | N | LEU | A | 540 | 994 | 1270 | 1216 | 24 | 13 | 23 | A | N |
| ATOM | 1993 | CA | LEU | A | 540 | −2.551 | −5.753 | 16.947 | 1.00 | 9.93 | | A | C |
| ANISOU | 1993 | CA | LEU | A | 540 | 1148 | 1347 | 1277 | 11 | −27 | 8 | A | C |
| ATOM | 1995 | CB | LEU | A | 540 | −1.383 | −4.793 | 16.676 | 1.00 | 10.30 | | A | C |
| ANISOU | 1995 | CB | LEU | A | 540 | 1226 | 1342 | 1344 | −27 | 43 | 27 | A | C |
| ATOM | 1998 | CG | LEU | A | 540 | −1.357 | −3.503 | 17.527 | 1.00 | 11.55 | | A | C |
| ANISOU | 1998 | CG | LEU | A | 540 | 1448 | 1487 | 1453 | 24 | 27 | 10 | A | C |
| ATOM | 2000 | CD1 | LEU | A | 540 | −0.182 | −2.587 | 17.170 | 1.00 | 13.03 | | A | C |
| ANISOU | 2000 | CD1 | LEU | A | 540 | 1510 | 1623 | 1818 | 12 | 59 | −30 | A | C |
| ATOM | 2004 | CD2 | LEU | A | 540 | −2.683 | −2.769 | 17.444 | 1.00 | 10.93 | | A | C |
| ANISOU | 2004 | CD2 | LEU | A | 540 | 1550 | 1247 | 1357 | 92 | −1 | −6 | A | C |
| ATOM | 2008 | C | LEU | A | 540 | −2.607 | −6.914 | 15.941 | 1.00 | 9.95 | | A | C |
| ANISOU | 2008 | C | LEU | A | 540 | 1155 | 1333 | 1291 | 37 | 17 | 7 | A | C |
| ATOM | 2009 | O | LEU | A | 540 | −3.339 | −6.847 | 14.957 | 1.00 | 10.84 | | A | O |
| ANISOU | 2009 | O | LEU | A | 540 | 1157 | 1574 | 1385 | 137 | −103 | −57 | A | O |
| ATOM | 2010 | N | GLU | A | 541 | −1.883 | −7.994 | 16.183 | 1.00 | 10.20 | | A | N |
| ANISOU | 2010 | N | GLU | A | 541 | 1222 | 1390 | 1263 | 21 | −127 | 20 | A | N |
| ATOM | 2012 | CA | GLU | A | 541 | −1.942 | −9.177 | 15.318 | 1.00 | 10.92 | | A | C |
| ANISOU | 2012 | CA | GLU | A | 541 | 1338 | 1411 | 1397 | 77 | 13 | −17 | A | C |
| ATOM | 2014 | CB | GLU | A | 541 | −1.027 | −10.256 | 15.898 | 1.00 | 11.79 | | A | C |
| ANISOU | 2014 | CB | GLU | A | 541 | 1402 | 1554 | 1523 | 95 | −62 | −29 | A | C |
| ATOM | 2017 | CG | GLU | A | 541 | −1.082 | −11.616 | 15.227 | 1.00 | 11.44 | | A | C |
| ANISOU | 2017 | CG | GLU | A | 541 | 1248 | 1518 | 1580 | −19 | −74 | 21 | A | C |
| ATOM | 2020 | CD | GLU | A | 541 | −0.083 | −12.570 | 15.866 | 1.00 | 12.04 | | A | C |
| ANISOU | 2020 | CD | GLU | A | 541 | 1463 | 1421 | 1690 | −44 | −118 | 32 | A | C |
| ATOM | 2021 | OE1 | GLU | A | 541 | −0.394 | −13.132 | 16.937 | 1.00 | 13.20 | | A | O |
| ANISOU | 2021 | OE1 | GLU | A | 541 | 1418 | 1892 | 1704 | 30 | −170 | 155 | A | O |
| ATOM | 2022 | OE2 | GLU | A | 541 | 1.026 | −12.690 | 15.331 | 1.00 | 12.85 | | A | O |
| ANISOU | 2022 | OE2 | GLU | A | 541 | 1700 | 1601 | 1579 | 52 | −98 | 24 | A | O |
| ATOM | 2023 | C | GLU | A | 541 | −3.384 | −9.717 | 15.198 | 1.00 | 11.01 | | A | C |
| ANISOU | 2023 | C | GLU | A | 541 | 1417 | 1423 | 1343 | 15 | −37 | −51 | A | C |
| ATOM | 2024 | O | GLU | A | 541 | −3.814 | −10.155 | 14.129 | 1.00 | 11.07 | | A | O |
| ANISOU | 2024 | O | GLU | A | 541 | 1329 | 1472 | 1405 | 115 | −37 | −127 | A | O |
| ATOM | 2025 | N | SER | A | 542 | −4.131 | −9.666 | 16.305 | 1.00 | 11.07 | | A | N |
| ANISOU | 2025 | N | SER | A | 542 | 1333 | 1456 | 1416 | −4 | 25 | −35 | A | N |
| ATOM | 2027 | CA | SER | A | 542 | −5.476 | −10.210 | 16.354 | 1.00 | 11.62 | | A | C |
| ANISOU | 2027 | CA | SER | A | 542 | 1396 | 1549 | 1468 | −28 | −12 | −29 | A | C |
| ATOM | 2029 | CB | SER | A | 542 | −6.005 | −10.237 | 17.795 | 1.00 | 11.54 | | A | C |
| ANISOU | 2029 | CB | SER | A | 542 | 1344 | 1574 | 1466 | −76 | −6 | −61 | A | C |
| ATOM | 2032 | OG | SER | A | 542 | −6.484 | −8.971 | 18.196 | 1.00 | 12.72 | | A | O |
| ANISOU | 2032 | OG | SER | A | 542 | 1330 | 1755 | 1747 | 50 | 49 | 71 | A | O |
| ATOM | 2034 | C | SER | A | 542 | −6.447 | −9.463 | 15.454 | 1.00 | 12.29 | | A | C |
| ANISOU | 2034 | C | SER | A | 542 | 1429 | 1642 | 1599 | −30 | −14 | −12 | A | C |
| ATOM | 2035 | O | SER | A | 542 | −7.479 | −10.038 | 15.085 | 1.00 | 13.05 | | A | O |
| ANISOU | 2035 | O | SER | A | 542 | 1416 | 1883 | 1658 | −148 | −85 | −25 | A | O |
| ATOM | 2036 | N | ILE | A | 543 | −6.139 | −8.203 | 15.125 | 1.00 | 12.06 | | A | N |
| ANISOU | 2036 | N | ILE | A | 543 | 1377 | 1609 | 1595 | 0 | −50 | −3 | A | N |
| ATOM | 2038 | CA | ILE | A | 543 | −6.927 | −7.427 | 14.167 | 1.00 | 12.89 | | A | C |
| ANISOU | 2038 | CA | ILE | A | 543 | 1507 | 1753 | 1636 | −22 | −12 | 33 | A | C |
| ATOM | 2040 | CB | ILE | A | 543 | −7.411 | −6.101 | 14.786 | 1.00 | 12.91 | | A | C |
| ANISOU | 2040 | CB | ILE | A | 543 | 1456 | 1795 | 1651 | −11 | −59 | 38 | A | C |
| ATOM | 2042 | CG1 | ILE | A | 543 | −6.272 | −5.280 | 15.408 | 1.00 | 12.95 | | A | C |
| ANISOU | 2042 | CG1 | ILE | A | 543 | 1623 | 1687 | 1610 | −26 | −51 | −33 | A | C |
| ATOM | 2045 | CD1 | ILE | A | 543 | −6.660 | −3.826 | 15.644 | 1.00 | 14.46 | | A | C |
| ANISOU | 2045 | CD1 | ILE | A | 543 | 1779 | 1755 | 1957 | −27 | −7 | 16 | A | C |
| ATOM | 2049 | CG2 | ILE | A | 543 | −8.465 | −6.376 | 15.826 | 1.00 | 13.78 | | A | C |
| ANISOU | 2049 | CG2 | ILE | A | 543 | 1634 | 1876 | 1723 | −8 | 28 | 56 | A | C |
| ATOM | 2053 | C | ILE | A | 543 | −6.184 | −7.192 | 12.848 | 1.00 | 13.59 | | A | C |
| ANISOU | 2053 | C | ILE | A | 543 | 1727 | 1794 | 1639 | 65 | −11 | 29 | A | C |
| ATOM | 2054 | O | ILE | A | 543 | −6.597 | −6.361 | 12.034 | 1.00 | 14.94 | | A | O |
| ANISOU | 2054 | O | ILE | A | 543 | 1834 | 1982 | 1858 | 192 | 28 | 151 | A | O |
| ATOM | 2055 | N | ASN | A | 544 | −5.142 | −7.979 | 12.594 | 1.00 | 14.05 | | A | N |
| ANISOU | 2055 | N | ASN | A | 544 | 1657 | 1897 | 1781 | 79 | −75 | 20 | A | N |
| ATOM | 2057 | CA | ASN | A | 544 | −4.333 | −7.838 | 11.388 | 1.00 | 15.80 | | A | C |
| ANISOU | 2057 | CA | ASN | A | 544 | 1991 | 2045 | 1965 | 65 | −36 | 67 | A | C |
| ATOM | 2059 | CB | ASN | A | 544 | −5.136 | −8.340 | 10.164 | 1.00 | 17.02 | | A | C |
| ANISOU | 2059 | CB | ASN | A | 544 | 2179 | 2222 | 2064 | 22 | 4 | 13 | A | C |
| ATOM | 2062 | CG | ASN | A | 544 | −5.600 | −9.780 | 10.307 | 1.00 | 21.95 | | A | C |
| ANISOU | 2062 | CG | ASN | A | 544 | 2855 | 2660 | 2822 | −52 | −23 | 11 | A | C |
| ATOM | 2063 | OD1 | ASN | A | 544 | −4.820 | −10.658 | 10.687 | 1.00 | 26.38 | | A | O |
| ANISOU | 2063 | OD1 | ASN | A | 544 | 3448 | 3186 | 3389 | 242 | −102 | 106 | A | O |
| ATOM | 2064 | ND2 | ASN | A | 544 | −6.864 | −10.045 | 9.950 | 1.00 | 25.54 | | A | N |
| ANISOU | 2064 | ND2 | ASN | A | 544 | 3002 | 3389 | 3311 | −28 | −9 | −30 | A | N |
| ATOM | 2067 | C | ASN | A | 544 | −3.839 | −6.386 | 11.145 | 1.00 | 15.07 | | A | C |
| ANISOU | 2067 | C | ASN | A | 544 | 1894 | 1964 | 1866 | 91 | −6 | −2 | A | C |
| ATOM | 2068 | O | ASN | A | 544 | −3.784 | −5.914 | 9.982 | 1.00 | 16.47 | | A | O |
| ANISOU | 2068 | O | ASN | A | 544 | 2180 | 2149 | 1928 | 174 | 0 | 10 | A | O |
| ATOM | 2069 | N | CAS | A | 545 | −3.491 | −5.677 | 12.224 | 1.00 | 14.03 | | A | N |
| ANISOU | 2069 | N | CAS | A | 545 | 1635 | 1857 | 1837 | 69 | −40 | 25 | A | N |
| ATOM | 2072 | CA | CAS | A | 545 | −2.959 | −4.333 | 12.145 | 1.00 | 14.24 | | A | C |
| ANISOU | 2072 | CA | CAS | A | 545 | 1745 | 1849 | 1815 | 34 | −17 | 35 | A | C |
| ATOM | 2074 | CB | CAS | A | 545 | −3.448 | −3.554 | 13.346 | 1.00 | 15.02 | | A | C |
| ANISOU | 2074 | CB | CAS | A | 545 | 1838 | 1878 | 1988 | 126 | 45 | 43 | A | C |
| ATOM | 2077 | SG | CAS | A | 545 | −2.801 | −1.903 | 13.461 | 1.00 | 19.61 | | A | S |
| ANISOU | 2077 | SG | CAS | A | 545 | 2777 | 2151 | 2522 | 186 | 47 | −59 | A | S |
| ATOM | 2078 | AS | CAS | A | 545 | −3.902 | −0.995 | 11.780 | 0.50 | 18.37 | | A | AS |
| ANISOU | 2078 | AS | CAS | A | 545 | 2487 | 1666 | 2824 | 158 | −103 | 26 | A | AS |
| ATOM | 2079 | CE2 | CAS | A | 545 | −2.576 | −0.647 | 10.336 | 0.50 | 20.07 | | A | C |
| ANISOU | 2079 | CE2 | CAS | A | 545 | 2571 | 2431 | 2622 | 30 | −72 | 10 | A | C |
| ATOM | 2083 | CE1 | CAS | A | 545 | −5.056 | 0.534 | 12.278 | 0.50 | 19.01 | | A | C |
| ANISOU | 2083 | CE1 | CAS | A | 545 | 2429 | 2292 | 2501 | 113 | 41 | −93 | A | C |
| ATOM | 2087 | C | CAS | A | 545 | −1.460 | −4.413 | 12.155 | 1.00 | 13.38 | | A | C |
| ANISOU | 2087 | C | CAS | A | 545 | 1637 | 1815 | 1631 | 44 | 10 | 77 | A | C |
| ATOM | 2088 | O | CAS | A | 545 | −0.842 | −4.851 | 13.112 | 1.00 | 13.52 | | A | O |
| ANISOU | 2088 | O | CAS | A | 545 | 1605 | 1982 | 1548 | 57 | 61 | 268 | A | O |
| ATOM | 2090 | N | VAL | A | 546 | −0.859 | −3.933 | 11.077 | 1.00 | 12.64 | | A | N |
| ANISOU | 2090 | N | VAL | A | 546 | 1604 | 1635 | 1564 | 30 | 75 | 33 | A | N |
| ATOM | 2092 | CA | VAL | A | 546 | 0.573 | −3.935 | 10.899 | 1.00 | 12.23 | | A | C |
| ANISOU | 2092 | CA | VAL | A | 546 | 1538 | 1611 | 1495 | 23 | −29 | 30 | A | C |
| ATOM | 2094 | CB | VAL | A | 546 | 0.954 | −4.245 | 9.452 | 1.00 | 13.05 | | A | C |
| ANISOU | 2094 | CB | VAL | A | 546 | 1613 | 1713 | 1629 | −1 | 40 | 8 | A | C |
| ATOM | 2096 | CG1 | VAL | A | 546 | 2.485 | −4.277 | 9.302 | 1.00 | 13.38 | | A | C |
| ANISOU | 2096 | CG1 | VAL | A | 546 | 1660 | 1832 | 1590 | −66 | 88 | 116 | A | C |
| ATOM | 2100 | CG2 | VAL | A | 546 | 0.343 | −5.567 | 9.029 | 1.00 | 13.89 | | A | C |
| ANISOU | 2100 | CG2 | VAL | A | 546 | 1761 | 1847 | 1666 | 16 | 36 | −13 | A | C |
| ATOM | 2104 | C | VAL | A | 546 | 1.094 | −2.574 | 11.315 | 1.00 | 11.11 | | A | C |
| ANISOU | 2104 | C | VAL | A | 546 | 1416 | 1456 | 1345 | 100 | 99 | 15 | A | C |
| ATOM | 2105 | O | VAL | A | 546 | 0.762 | −1.552 | 10.731 | 1.00 | 13.61 | | A | O |
| ANISOU | 2105 | O | VAL | A | 546 | 1681 | 1729 | 1759 | 139 | 13 | 126 | A | O |
| ATOM | 2106 | N | HIS | A | 547 | 1.967 | −2.572 | 12.311 | 1.00 | 10.67 | | A | N |
| ANISOU | 2106 | N | HIS | A | 547 | 1441 | 1290 | 1322 | 105 | 26 | 21 | A | N |
| ATOM | 2108 | CA | HIS | A | 547 | 2.375 | −1.352 | 12.978 | 1.00 | 10.61 | | A | C |
| ANISOU | 2108 | CA | HIS | A | 547 | 1372 | 1346 | 1311 | 60 | −25 | −23 | A | C |
| ATOM | 2110 | CB | HIS | A | 547 | 2.737 | −1.689 | 14.403 | 1.00 | 11.17 | | A | C |
| ANISOU | 2110 | CB | HIS | A | 547 | 1466 | 1408 | 1369 | −5 | 14 | −20 | A | C |
| ATOM | 2113 | CG | HIS | A | 547 | 2.988 | −0.500 | 15.257 | 1.00 | 10.55 | | A | C |
| ANISOU | 2113 | CG | HIS | A | 547 | 1326 | 1212 | 1467 | 214 | −53 | 50 | A | C |
| ATOM | 2114 | ND1 | HIS | A | 547 | 4.222 | 0.091 | 15.347 | 1.00 | 12.47 | | A | N |
| ANISOU | 2114 | ND1 | HIS | A | 547 | 1647 | 1680 | 1409 | −129 | −130 | 10 | A | N |
| ATOM | 2116 | CE1 | HIS | A | 547 | 4.168 | 1.092 | 16.208 | 1.00 | 12.36 | | A | C |
| ANISOU | 2116 | CE1 | HIS | A | 547 | 1532 | 1430 | 1733 | 96 | 25 | −63 | A | C |
| ATOM | 2118 | NE2 | HIS | A | 547 | 2.921 | 1.215 | 16.623 | 1.00 | 13.17 | | A | N |
| ANISOU | 2118 | NE2 | HIS | A | 547 | 1459 | 1693 | 1850 | −47 | −9 | 0 | A | N |
| ATOM | 2120 | CD2 | HIS | A | 547 | 2.165 | 0.228 | 16.042 | 1.00 | 11.72 | | A | C |
| ANISOU | 2120 | CD2 | HIS | A | 547 | 1379 | 1402 | 1672 | 153 | 89 | −94 | A | C |
| ATOM | 2122 | C | HIS | A | 547 | 3.515 | −0.604 | 12.297 | 1.00 | 11.31 | | A | C |
| ANISOU | 2122 | C | HIS | A | 547 | 1513 | 1402 | 1379 | 42 | −12 | −8 | A | C |
| ATOM | 2123 | O | HIS | A | 547 | 3.443 | 0.608 | 12.112 | 1.00 | 12.83 | | A | O |
| ANISOU | 2123 | O | HIS | A | 547 | 1672 | 1536 | 1665 | 78 | −21 | −30 | A | O |
| ATOM | 2124 | N | ARG | A | 548 | 4.564 | −1.342 | 11.938 | 1.00 | 11.57 | | A | N |
| ANISOU | 2124 | N | ARG | A | 548 | 1449 | 1460 | 1487 | 61 | −12 | 93 | A | N |
| ATOM | 2126 | CA | ARG | A | 548 | 5.723 | −0.839 | 11.208 | 1.00 | 11.95 | | A | C |
| ANISOU | 2126 | CA | ARG | A | 548 | 1411 | 1558 | 1568 | 50 | 24 | 42 | A | C |
| ATOM | 2128 | CB | ARG | A | 548 | 5.324 | −0.106 | 9.919 | 1.00 | 12.37 | | A | C |
| ANISOU | 2128 | CB | ARG | A | 548 | 1407 | 1690 | 1600 | 23 | 42 | 63 | A | C |
| ATOM | 2131 | CG | ARG | A | 548 | 4.400 | −0.860 | 9.000 | 1.00 | 12.27 | | A | C |
| ANISOU | 2131 | CG | ARG | A | 548 | 1419 | 1656 | 1584 | 58 | 73 | 1 | A | C |
| ATOM | 2134 | CD | ARG | A | 548 | 4.245 | −0.126 | 7.686 | 1.00 | 14.56 | | A | C |
| ANISOU | 2134 | CD | ARG | A | 548 | 1895 | 1865 | 1769 | 49 | 23 | 31 | A | C |
| ATOM | 2137 | NE | ARG | A | 548 | 3.276 | −0.702 | 6.768 | 1.00 | 14.69 | | A | N |
| ANISOU | 2137 | NE | ARG | A | 548 | 1963 | 1899 | 1720 | 78 | −18 | 120 | A | N |
| ATOM | 2139 | CZ | ARG | A | 548 | 3.282 | −0.444 | 5.464 | 1.00 | 15.09 | | A | C |
| ANISOU | 2139 | CZ | ARG | A | 548 | 2009 | 1966 | 1758 | −45 | 23 | −4 | A | C |
| ATOM | 2140 | NH1 | ARG | A | 548 | 4.174 | 0.391 | 4.974 | 1.00 | 14.76 | | A | N |
| ANISOU | 2140 | NH1 | ARG | A | 548 | 2013 | 2073 | 1519 | −47 | 62 | −21 | A | N |
| ATOM | 2143 | NH2 | ARG | A | 548 | 2.390 | −0.992 | 4.656 | 1.00 | 16.02 | | A | N |
| ANISOU | 2143 | NH2 | ARG | A | 548 | 1971 | 2124 | 1989 | −61 | 16 | −2 | A | N |
| ATOM | 2146 | C | ARG | A | 548 | 6.693 | 0.072 | 11.955 | 1.00 | 11.43 | | A | C |
| ANISOU | 2146 | C | ARG | A | 548 | 1385 | 1419 | 1538 | 46 | 35 | 92 | A | C |
| ATOM | 2147 | O | ARG | A | 548 | 7.658 | 0.531 | 11.349 | 1.00 | 13.42 | | A | O |
| ANISOU | 2147 | O | ARG | A | 548 | 1435 | 1793 | 1868 | 36 | 56 | 223 | A | O |
| ATOM | 2148 | N | ASP | A | 549 | 6.448 | 0.383 | 13.222 | 1.00 | 12.01 | | A | N |
| ANISOU | 2148 | N | ASP | A | 549 | 1462 | 1490 | 1609 | 38 | 34 | 30 | A | N |
| ATOM | 2150 | CA | ASP | A | 549 | 7.341 | 1.272 | 13.975 | 1.00 | 12.94 | | A | C |
| ANISOU | 2150 | CA | ASP | A | 549 | 1595 | 1591 | 1729 | 97 | −48 | −30 | A | C |
| ATOM | 2152 | CB | ASP | A | 549 | 6.878 | 2.731 | 13.931 | 1.00 | 14.09 | | A | C |
| ANISOU | 2152 | CB | ASP | A | 549 | 1785 | 1668 | 1897 | 107 | −31 | −8 | A | C |
| ATOM | 2155 | CG | ASP | A | 549 | 7.986 | 3.712 | 14.315 | 1.00 | 17.92 | | A | C |
| ANISOU | 2155 | CG | ASP | A | 549 | 2227 | 2112 | 2468 | −45 | −18 | −21 | A | C |
| ATOM | 2156 | OD1 | ASP | A | 549 | 9.171 | 3.291 | 14.479 | 1.00 | 21.77 | | A | O |
| ANISOU | 2156 | OD1 | ASP | A | 549 | 2568 | 2693 | 3008 | 6 | −101 | 112 | A | O |
| ATOM | 2157 | OD2 | ASP | A | 549 | 7.742 | 4.937 | 14.480 | 1.00 | 20.33 | | A | O |
| ANISOU | 2157 | OD2 | ASP | A | 549 | 2780 | 2082 | 2862 | −148 | 25 | −17 | A | O |
| ATOM | 2158 | C | ASP | A | 549 | 7.551 | 0.816 | 15.392 | 1.00 | 12.24 | | A | C |
| ANISOU | 2158 | C | ASP | A | 549 | 1466 | 1528 | 1654 | 51 | 0 | −32 | A | C |
| ATOM | 2159 | O | ASP | A | 549 | 7.316 | 1.550 | 16.367 | 1.00 | 13.22 | | A | O |
| ANISOU | 2159 | O | ASP | A | 549 | 1691 | 1621 | 1707 | 143 | −155 | −110 | A | O |
| ATOM | 2160 | N | ILE | A | 550 | 8.025 | −0.419 | 15.499 | 1.00 | 11.46 | | A | N |
| ANISOU | 2160 | N | ILE | A | 550 | 1425 | 1417 | 1512 | 57 | −51 | −47 | A | N |
| ATOM | 2162 | CA | ILE | A | 550 | 8.294 | −1.047 | 16.778 | 1.00 | 10.66 | | A | C |
| ANISOU | 2162 | CA | ILE | A | 550 | 1268 | 1415 | 1364 | 33 | −30 | −53 | A | C |
| ATOM | 2164 | CB | ILE | A | 550 | 7.904 | −2.528 | 16.745 | 1.00 | 10.39 | | A | C |
| ANISOU | 2164 | CB | ILE | A | 550 | 1208 | 1351 | 1388 | 60 | 31 | −26 | A | C |
| ATOM | 2166 | CG1 | ILE | A | 550 | 6.497 | −2.712 | 16.183 | 1.00 | 11.03 | | A | C |
| ANISOU | 2166 | CG1 | ILE | A | 550 | 1285 | 1371 | 1532 | −38 | −61 | −29 | A | C |
| ATOM | 2169 | CD1 | ILE | A | 550 | 6.253 | −4.071 | 15.615 | 1.00 | 13.07 | | A | C |
| ANISOU | 2169 | CD1 | ILE | A | 550 | 1555 | 1625 | 1784 | −13 | 56 | −101 | A | C |
| ATOM | 2173 | CG2 | ILE | A | 550 | 7.997 | −3.106 | 18.142 | 1.00 | 12.08 | | A | C |
| ANISOU | 2173 | CG2 | ILE | A | 550 | 1455 | 1598 | 1536 | 164 | −6 | 62 | A | C |
| ATOM | 2177 | C | ILE | A | 550 | 9.791 | −0.900 | 17.051 | 1.00 | 10.76 | | A | C |
| ANISOU | 2177 | C | ILE | A | 550 | 1253 | 1424 | 1410 | 79 | −60 | −75 | A | C |
| ATOM | 2178 | O | ILE | A | 550 | 10.636 | −1.635 | 16.527 | 1.00 | 11.52 | | A | O |
| ANISOU | 2178 | O | ILE | A | 550 | 1347 | 1415 | 1612 | 152 | −124 | −125 | A | O |
| ATOM | 2179 | N | ALA | A | 551 | 10.109 | 0.131 | 17.820 | 1.00 | 10.63 | | A | N |
| ANISOU | 2179 | N | ALA | A | 551 | 1251 | 1325 | 1461 | 83 | −64 | −113 | A | N |
| ATOM | 2181 | CA | ALA | A | 551 | 11.484 | 0.550 | 18.071 | 1.00 | 10.00 | | A | C |
| ANISOU | 2181 | CA | ALA | A | 551 | 1231 | 1250 | 1316 | −1 | −10 | 4 | A | C |
| ATOM | 2183 | CB | ALA | A | 551 | 11.996 | 1.385 | 16.909 | 1.00 | 9.71 | | A | C |
| ANISOU | 2183 | CB | ALA | A | 551 | 1162 | 1199 | 1328 | 31 | 97 | −38 | A | C |
| ATOM | 2187 | C | ALA | A | 551 | 11.503 | 1.365 | 19.339 | 1.00 | 9.46 | | A | C |
| ANISOU | 2187 | C | ALA | A | 551 | 1166 | 1222 | 1206 | −13 | 39 | 33 | A | C |
| ATOM | 2188 | O | ALA | A | 551 | 10.511 | 1.990 | 19.683 | 1.00 | 11.05 | | A | O |
| ANISOU | 2188 | O | ALA | A | 551 | 1324 | 1408 | 1464 | 6 | −41 | 22 | A | O |
| ATOM | 2189 | N | VAL | A | 552 | 12.635 | 1.376 | 20.041 | 1.00 | 9.14 | | A | N |
| ANISOU | 2189 | N | VAL | A | 552 | 1102 | 1081 | 1290 | 47 | 5 | 12 | A | N |
| ATOM | 2191 | CA | VAL | A | 552 | 12.676 | 2.000 | 21.365 | 1.00 | 9.60 | | A | C |
| ANISOU | 2191 | CA | VAL | A | 552 | 1211 | 1209 | 1227 | −10 | −2 | 34 | A | C |
| ATOM | 2193 | CB | VAL | A | 552 | 13.970 | 1.684 | 22.161 | 1.00 | 9.79 | | A | C |
| ANISOU | 2193 | CB | VAL | A | 552 | 1144 | 1219 | 1354 | 59 | 24 | −80 | A | C |
| ATOM | 2195 | CG1 | VAL | A | 552 | 14.040 | 0.209 | 22.482 | 1.00 | 10.22 | | A | C |
| ANISOU | 2195 | CG1 | VAL | A | 552 | 1205 | 1269 | 1408 | 7 | 50 | 124 | A | C |
| ATOM | 2199 | CG2 | VAL | A | 552 | 15.209 | 2.196 | 21.483 | 1.00 | 9.58 | | A | C |
| ANISOU | 2199 | CG2 | VAL | A | 552 | 1119 | 1259 | 1260 | −6 | −30 | −85 | A | C |
| ATOM | 2203 | C | VAL | A | 552 | 12.450 | 3.506 | 21.350 | 1.00 | 10.58 | | A | C |
| ANISOU | 2203 | C | VAL | A | 552 | 1321 | 1273 | 1426 | −3 | 55 | −57 | A | C |
| ATOM | 2204 | O | VAL | A | 552 | 11.957 | 4.074 | 22.343 | 1.00 | 11.27 | | A | O |
| ANISOU | 2204 | O | VAL | A | 552 | 1553 | 1429 | 1301 | −2 | −9 | −61 | A | O |
| ATOM | 2205 | N | ARG | A | 553 | 12.780 | 4.150 | 20.234 | 1.00 | 11.21 | | A | N |
| ANISOU | 2205 | N | ARG | A | 553 | 1477 | 1382 | 1400 | 14 | 22 | −86 | A | N |
| ATOM | 2207 | CA | ARG | A | 553 | 12.497 | 5.572 | 20.061 | 1.00 | 12.16 | | A | C |
| ANISOU | 2207 | CA | ARG | A | 553 | 1563 | 1432 | 1624 | 27 | 22 | −24 | A | C |
| ATOM | 2209 | CB | ARG | A | 553 | 13.143 | 6.078 | 18.762 | 1.00 | 13.88 | | A | C |
| ANISOU | 2209 | CB | ARG | A | 553 | 1762 | 1701 | 1810 | −26 | 73 | 47 | A | C |
| ATOM | 2212 | CG | ARG | A | 553 | 12.998 | 7.551 | 18.453 | 1.00 | 18.30 | | A | C |
| ANISOU | 2212 | CG | ARG | A | 553 | 2397 | 2089 | 2467 | 108 | 26 | −58 | A | C |
| ATOM | 2215 | CD | ARG | A | 553 | 13.731 | 7.938 | 17.160 | 1.00 | 23.71 | | A | C |
| ANISOU | 2215 | CD | ARG | A | 553 | 3003 | 3086 | 2919 | 2 | 123 | 87 | A | C |
| ATOM | 2218 | NE | ARG | A | 553 | 13.755 | 9.366 | 16.854 | 1.00 | 28.74 | | A | N |
| ANISOU | 2218 | NE | ARG | A | 553 | 3758 | 3444 | 3716 | 39 | 11 | 10 | A | N |
| ATOM | 2220 | CZ | ARG | A | 553 | 14.289 | 9.867 | 15.739 | 1.00 | 31.75 | | A | C |
| ANISOU | 2220 | CZ | ARG | A | 553 | 4090 | 4001 | 3971 | −14 | 71 | 59 | A | C |
| ATOM | 2221 | NH1 | ARG | A | 553 | 14.846 | 9.063 | 14.832 | 1.00 | 33.43 | | A | N |
| ANISOU | 2221 | NH1 | ARG | A | 553 | 4320 | 4210 | 4169 | 56 | 86 | −3 | A | N |
| ATOM | 2224 | NH2 | ARG | A | 553 | 14.265 | 11.172 | 15.519 | 1.00 | 32.88 | | A | N |
| ANISOU | 2224 | NH2 | ARG | A | 553 | 4281 | 4027 | 4185 | −14 | 48 | 26 | A | N |
| ATOM | 2227 | C | ARG | A | 553 | 10.973 | 5.833 | 20.053 | 1.00 | 11.70 | | A | C |
| ANISOU | 2227 | C | ARG | A | 553 | 1522 | 1383 | 1538 | 48 | 10 | −29 | A | C |
| ATOM | 2228 | O | ARG | A | 553 | 10.533 | 6.955 | 20.301 | 1.00 | 13.49 | | A | O |
| ANISOU | 2228 | O | ARG | A | 553 | 1766 | 1425 | 1932 | 29 | 37 | −158 | A | O |
| ATOM | 2229 | N | ASN | A | 554 | 10.197 | 4.801 | 19.754 | 1.00 | 11.17 | | A | N |
| ANISOU | 2229 | N | ASN | A | 554 | 1357 | 1334 | 1550 | 21 | 65 | −9 | A | N |
| ATOM | 2231 | CA | ASN | A | 554 | 8.738 | 4.884 | 19.635 | 1.00 | 10.72 | | A | C |
| ANISOU | 2231 | CA | ASN | A | 554 | 1330 | 1319 | 1424 | −13 | 17 | −55 | A | C |
| ATOM | 2233 | CB | ASN | A | 554 | 8.249 | 4.432 | 18.249 | 1.00 | 10.62 | | A | C |
| ANISOU | 2233 | CB | ASN | A | 554 | 1364 | 1352 | 1318 | −1 | 24 | 6 | A | C |
| ATOM | 2236 | CG | ASN | A | 554 | 6.807 | 4.871 | 17.959 | 1.00 | 12.01 | | A | C |
| ANISOU | 2236 | CG | ASN | A | 554 | 1478 | 1588 | 1496 | 29 | 19 | −117 | A | C |
| ATOM | 2237 | OD1 | ASN | A | 554 | 6.403 | 5.973 | 18.358 | 1.00 | 15.71 | | A | O |
| ANISOU | 2237 | OD1 | ASN | A | 554 | 1886 | 1664 | 2416 | 172 | 72 | −49 | A | O |
| ATOM | 2238 | ND2 | ASN | A | 554 | 6.032 | 4.018 | 17.285 | 1.00 | 12.82 | | A | N |
| ANISOU | 2238 | ND2 | ASN | A | 554 | 1674 | 1585 | 1609 | −3 | 135 | −79 | A | N |
| ATOM | 2241 | C | ASN | A | 554 | 8.007 | 4.096 | 20.708 | 1.00 | 10.60 | | A | C |
| ANISOU | 2241 | C | ASN | A | 554 | 1285 | 1399 | 1341 | −2 | 59 | −65 | A | C |
| ATOM | 2242 | O | ASN | A | 554 | 6.889 | 3.651 | 20.506 | 1.00 | 11.82 | | A | O |
| ANISOU | 2242 | O | ASN | A | 554 | 1334 | 1668 | 1489 | −37 | 93 | −58 | A | O |
| ATOM | 2243 | N | ILE | A | 555 | 8.660 | 3.897 | 21.840 | 1.00 | 10.92 | | A | N |
| ANISOU | 2243 | N | ILE | A | 555 | 1279 | 1524 | 1344 | −102 | 94 | 22 | A | N |
| ATOM | 2245 | CA | ILE | A | 555 | 8.053 | 3.329 | 23.036 | 1.00 | 10.52 | | A | C |
| ANISOU | 2245 | CA | ILE | A | 555 | 1291 | 1427 | 1278 | −43 | 91 | −30 | A | C |
| ATOM | 2247 | CB | ILE | A | 555 | 8.726 | 2.024 | 23.412 | 1.00 | 10.98 | | A | C |
| ANISOU | 2247 | CB | ILE | A | 555 | 1399 | 1514 | 1258 | −86 | 80 | 5 | A | C |
| ATOM | 2249 | CG1 | ILE | A | 555 | 8.482 | 0.986 | 22.302 | 1.00 | 12.83 | | A | C |
| ANISOU | 2249 | CG1 | ILE | A | 555 | 1727 | 1630 | 1516 | −14 | 167 | −14 | A | C |
| ATOM | 2252 | CD1 | ILE | A | 555 | 9.354 | −0.148 | 22.390 | 1.00 | 13.57 | | A | C |
| ANISOU | 2252 | CD1 | ILE | A | 555 | 1552 | 1857 | 1745 | 0 | 106 | −62 | A | C |
| ATOM | 2256 | CG2 | ILE | A | 555 | 8.210 | 1.549 | 24.788 | 1.00 | 11.79 | | A | C |
| ANISOU | 2256 | CG2 | ILE | A | 555 | 1433 | 1656 | 1389 | −19 | 194 | 59 | A | C |
| ATOM | 2260 | C | ILE | A | 555 | 8.206 | 4.369 | 24.127 | 1.00 | 10.30 | | A | C |
| ANISOU | 2260 | C | ILE | A | 555 | 1241 | 1376 | 1295 | −57 | 13 | −9 | A | C |
| ATOM | 2261 | O | ILE | A | 555 | 9.261 | 4.978 | 24.271 | 1.00 | 10.45 | | A | O |
| ANISOU | 2261 | O | ILE | A | 555 | 1336 | 1295 | 1337 | −131 | 107 | −9 | A | O |
| ATOM | 2262 | N | LEU | A | 556 | 7.125 | 4.598 | 24.858 | 1.00 | 10.78 | | A | N |
| ANISOU | 2262 | N | LEU | A | 556 | 1288 | 1442 | 1364 | −37 | 39 | −96 | A | N |
| ATOM | 2264 | CA | LEU | A | 556 | 7.109 | 5.589 | 25.924 | 1.00 | 11.49 | | A | C |
| ANISOU | 2264 | CA | LEU | A | 556 | 1447 | 1438 | 1479 | −15 | 37 | −69 | A | C |
| ATOM | 2266 | CB | LEU | A | 556 | 5.922 | 6.560 | 25.755 | 1.00 | 12.91 | | A | C |
| ANISOU | 2266 | CB | LEU | A | 556 | 1553 | 1603 | 1748 | 76 | 34 | −120 | A | C |
| ATOM | 2269 | CG | LEU | A | 556 | 5.973 | 7.473 | 24.521 | 1.00 | 16.23 | | A | C |
| ANISOU | 2269 | CG | LEU | A | 556 | 1988 | 2084 | 2094 | 48 | 54 | 26 | A | C |
| ATOM | 2271 | CD1 | LEU | A | 556 | 4.806 | 8.420 | 24.478 | 1.00 | 20.11 | | A | C |
| ANISOU | 2271 | CD1 | LEU | A | 556 | 2429 | 2590 | 2621 | 132 | −85 | 24 | A | C |
| ATOM | 2275 | CD2 | LEU | A | 556 | 7.232 | 8.281 | 24.391 | 1.00 | 18.33 | | A | C |
| ANISOU | 2275 | CD2 | LEU | A | 556 | 2313 | 2157 | 2494 | −49 | −71 | 37 | A | C |
| ATOM | 2279 | C | LEU | A | 556 | 7.099 | 4.924 | 27.264 | 1.00 | 11.18 | | A | C |
| ANISOU | 2279 | C | LEU | A | 556 | 1411 | 1350 | 1486 | −8 | 5 | −68 | A | C |
| ATOM | 2280 | O | LEU | A | 556 | 6.497 | 3.875 | 27.443 | 1.00 | 10.88 | | A | O |
| ANISOU | 2280 | O | LEU | A | 556 | 1435 | 1321 | 1375 | −47 | 107 | −217 | A | O |
| ATOM | 2281 | N | VAL | A | 557 | 7.798 | 5.531 | 28.216 | 1.00 | 10.68 | | A | N |
| ANISOU | 2281 | N | VAL | A | 557 | 1407 | 1295 | 1354 | −64 | 15 | −94 | A | N |
| ATOM | 2283 | CA | VAL | A | 557 | 7.917 | 4.967 | 29.558 | 1.00 | 10.69 | | A | C |
| ANISOU | 2283 | CA | VAL | A | 557 | 1364 | 1324 | 1373 | 10 | −2 | −46 | A | C |
| ATOM | 2285 | CB | VAL | A | 557 | 9.334 | 5.102 | 30.117 | 1.00 | 10.98 | | A | C |
| ANISOU | 2285 | CB | VAL | A | 557 | 1382 | 1355 | 1434 | −61 | 39 | −51 | A | C |
| ATOM | 2287 | CG1 | VAL | A | 557 | 9.415 | 4.495 | 31.502 | 1.00 | 10.58 | | A | C |
| ANISOU | 2287 | CG1 | VAL | A | 557 | 1261 | 1241 | 1516 | 73 | 32 | −44 | A | C |
| ATOM | 2291 | CG2 | VAL | A | 557 | 10.350 | 4.474 | 29.149 | 1.00 | 11.50 | | A | C |
| ANISOU | 2291 | CG2 | VAL | A | 557 | 1411 | 1344 | 1611 | −21 | 0 | −106 | A | C |
| ATOM | 2295 | C | VAL | A | 557 | 6.916 | 5.690 | 30.481 | 1.00 | 11.26 | | A | C |
| ANISOU | 2295 | C | VAL | A | 557 | 1376 | 1443 | 1460 | 1 | 26 | −82 | A | C |
| ATOM | 2296 | O | VAL | A | 557 | 7.056 | 6.887 | 30.794 | 1.00 | 11.64 | | A | O |
| ANISOU | 2296 | O | VAL | A | 557 | 1282 | 1456 | 1684 | 73 | −4 | −146 | A | O |
| ATOM | 2297 | N | ALA | A | 558 | 5.872 | 4.974 | 30.860 | 1.00 | 12.16 | | A | N |
| ANISOU | 2297 | N | ALA | A | 558 | 1549 | 1560 | 1509 | −76 | −6 | −103 | A | N |
| ATOM | 2299 | CA | ALA | A | 558 | 4.879 | 5.498 | 31.793 | 1.00 | 12.50 | | A | C |
| ANISOU | 2299 | CA | ALA | A | 558 | 1508 | 1649 | 1592 | 28 | 24 | −50 | A | C |
| ATOM | 2301 | CB | ALA | A | 558 | 3.598 | 4.672 | 31.704 | 1.00 | 12.80 | | A | C |
| ANISOU | 2301 | CB | ALA | A | 558 | 1634 | 1655 | 1573 | −49 | 6 | −47 | A | C |
| ATOM | 2305 | C | ALA | A | 558 | 5.390 | 5.485 | 33.238 | 1.00 | 12.51 | | A | C |
| ANISOU | 2305 | C | ALA | A | 558 | 1535 | 1663 | 1552 | 47 | −31 | −122 | A | C |
| ATOM | 2306 | O | ALA | A | 558 | 5.104 | 6.380 | 34.066 | 1.00 | 14.27 | | A | O |
| ANISOU | 2306 | O | ALA | A | 558 | 1703 | 1875 | 1841 | 126 | −98 | −181 | A | O |
| ATOM | 2307 | N | SER | A | 559 | 6.096 | 4.421 | 33.574 | 1.00 | 12.55 | | A | N |
| ANISOU | 2307 | N | SER | A | 559 | 1564 | 1624 | 1580 | 5 | −8 | −68 | A | N |
| ATOM | 2309 | CA | SER | A | 559 | 6.722 | 4.284 | 34.883 | 1.00 | 12.69 | | A | C |
| ANISOU | 2309 | CA | SER | A | 559 | 1603 | 1618 | 1599 | −24 | 6 | −60 | A | C |
| ATOM | 2311 | CB | SER | A | 559 | 5.695 | 3.793 | 35.901 | 1.00 | 12.95 | | A | C |
| ANISOU | 2311 | CB | SER | A | 559 | 1673 | 1634 | 1613 | 9 | 34 | 1 | A | C |
| ATOM | 2314 | OG | SER | A | 559 | 5.391 | 2.428 | 35.687 | 1.00 | 14.31 | | A | O |
| ANISOU | 2314 | OG | SER | A | 559 | 1859 | 1733 | 1843 | 0 | 192 | −165 | A | O |
| ATOM | 2316 | C | SER | A | 559 | 7.828 | 3.264 | 34.747 | 1.00 | 12.56 | | A | C |
| ANISOU | 2316 | C | SER | A | 559 | 1543 | 1639 | 1587 | −22 | 51 | −34 | A | C |
| ATOM | 2317 | O | SER | A | 559 | 7.885 | 2.580 | 33.719 | 1.00 | 12.75 | | A | O |
| ANISOU | 2317 | O | SER | A | 559 | 1590 | 1693 | 1560 | −111 | 69 | −101 | A | O |
| ATOM | 2318 | N | PRO | A | 560 | 8.696 | 3.113 | 35.742 | 1.00 | 12.53 | | A | N |
| ANISOU | 2318 | N | PRO | A | 560 | 1577 | 1600 | 1582 | −19 | 21 | −83 | A | N |
| ATOM | 2319 | CA | PRO | A | 560 | 9.684 | 2.019 | 35.688 | 1.00 | 13.14 | | A | C |
| ANISOU | 2319 | CA | PRO | A | 560 | 1654 | 1704 | 1633 | 23 | 25 | −36 | A | C |
| ATOM | 2321 | CB | PRO | A | 560 | 10.423 | 2.146 | 37.011 | 1.00 | 13.21 | | A | C |
| ANISOU | 2321 | CB | PRO | A | 560 | 1593 | 1721 | 1705 | 46 | 0 | −50 | A | C |
| ATOM | 2324 | CG | PRO | A | 560 | 10.289 | 3.595 | 37.339 | 1.00 | 13.53 | | A | C |
| ANISOU | 2324 | CG | PRO | A | 560 | 1632 | 1733 | 1775 | −64 | −20 | −70 | A | C |
| ATOM | 2327 | CD | PRO | A | 560 | 8.892 | 3.966 | 36.929 | 1.00 | 13.11 | | A | C |
| ANISOU | 2327 | CD | PRO | A | 560 | 1688 | 1704 | 1586 | 63 | −3 | −27 | A | C |
| ATOM | 2330 | C | PRO | A | 560 | 9.064 | 0.619 | 35.507 | 1.00 | 13.46 | | A | C |
| ANISOU | 2330 | C | PRO | A | 560 | 1734 | 1706 | 1674 | 25 | 26 | −53 | A | C |
| ATOM | 2331 | O | PRO | A | 560 | 9.781 | −0.325 | 35.091 | 1.00 | 14.90 | | A | O |
| ANISOU | 2331 | O | PRO | A | 560 | 2001 | 1925 | 1736 | 102 | 149 | −117 | A | O |
| ATOM | 2332 | N | GLU | A | 561 | 7.764 | 0.499 | 35.784 | 1.00 | 14.34 | | A | N |
| ANISOU | 2332 | N | GLU | A | 561 | 1839 | 1877 | 1732 | −8 | 27 | −80 | A | N |
| ATOM | 2334 | CA | GLU | A | 561 | 7.047 | −0.771 | 35.718 | 1.00 | 14.84 | | A | C |
| ANISOU | 2334 | CA | GLU | A | 561 | 1929 | 1900 | 1809 | −27 | 14 | −57 | A | C |
| ATOM | 2336 | CB | GLU | A | 561 | 6.041 | −0.890 | 36.883 | 1.00 | 16.70 | | A | C |
| ANISOU | 2336 | CB | GLU | A | 561 | 2112 | 2109 | 2124 | −109 | 61 | −10 | A | C |
| ATOM | 2339 | CG | GLU | A | 561 | 6.678 | −0.972 | 38.251 | 1.00 | 20.63 | | A | C |
| ANISOU | 2339 | CG | GLU | A | 561 | 2661 | 2689 | 2487 | −4 | −55 | 25 | A | C |
| ATOM | 2342 | CD | GLU | A | 561 | 7.188 | 0.367 | 38.748 | 1.00 | 25.39 | | A | C |
| ANISOU | 2342 | CD | GLU | A | 561 | 3275 | 3015 | 3357 | −100 | −42 | −45 | A | C |
| ATOM | 2343 | OE1 | GLU | A | 561 | 6.484 | 1.405 | 38.583 | 1.00 | 27.09 | | A | O |
| ANISOU | 2343 | OE1 | GLU | A | 561 | 3289 | 3365 | 3639 | 65 | −24 | 69 | A | O |
| ATOM | 2344 | OE2 | GLU | A | 561 | 8.308 | 0.373 | 39.309 | 1.00 | 29.08 | | A | O |
| ANISOU | 2344 | OE2 | GLU | A | 561 | 3480 | 3721 | 3845 | −62 | −156 | 52 | A | O |
| ATOM | 2345 | C | GLU | A | 561 | 6.245 | −0.953 | 34.440 | 1.00 | 13.68 | | A | C |
| ANISOU | 2345 | C | GLU | A | 561 | 1749 | 1762 | 1684 | −21 | 39 | −34 | A | C |
| ATOM | 2346 | O | GLU | A | 561 | 5.624 | −1.988 | 34.275 | 1.00 | 13.37 | | A | O |
| ANISOU | 2346 | O | GLU | A | 561 | 1859 | 1716 | 1503 | −42 | 73 | −194 | A | O |
| ATOM | 2347 | N | CYS | A | 562 | 6.216 | 0.040 | 33.565 | 1.00 | 12.49 | | A | N |
| ANISOU | 2347 | N | CYS | A | 562 | 1594 | 1673 | 1479 | −68 | 24 | −56 | A | N |
| ATOM | 2349 | CA | CYS | A | 562 | 5.275 | −0.001 | 32.455 | 1.00 | 13.84 | | A | C |
| ANISOU | 2349 | CA | CYS | A | 562 | 1726 | 1863 | 1668 | −17 | 34 | 0 | A | C |
| ATOM | 2351 | CB | CYS | A | 562 | 3.902 | 0.495 | 32.925 | 1.00 | 14.48 | | A | C |
| ANISOU | 2351 | CB | CYS | A | 562 | 1810 | 1887 | 1802 | −6 | 54 | −57 | A | C |
| ATOM | 2354 | SG | CYS | A | 562 | 2.661 | 0.449 | 31.631 | 1.00 | 17.07 | | A | S |
| ANISOU | 2354 | SG | CYS | A | 562 | 1661 | 2589 | 2234 | 89 | 144 | −150 | A | S |
| ATOM | 2355 | C | CYS | A | 562 | 5.703 | 0.831 | 31.253 | 1.00 | 12.03 | | A | C |
| ANISOU | 2355 | C | CYS | A | 562 | 1505 | 1592 | 1471 | −22 | 53 | −32 | A | C |
| ATOM | 2356 | O | CYS | A | 562 | 5.878 | 2.048 | 31.345 | 1.00 | 11.76 | | A | O |
| ANISOU | 2356 | O | CYS | A | 562 | 1560 | 1728 | 1180 | −40 | 142 | −131 | A | O |
| ATOM | 2357 | N | VAL | A | 563 | 5.829 | 0.158 | 30.111 | 1.00 | 11.30 | | A | N |
| ANISOU | 2357 | N | VAL | A | 563 | 1394 | 1522 | 1374 | 48 | 42 | −32 | A | N |
| ATOM | 2359 | CA | VAL | A | 563 | 6.071 | 0.838 | 28.857 | 1.00 | 10.98 | | A | C |
| ANISOU | 2359 | CA | VAL | A | 563 | 1335 | 1426 | 1408 | 30 | 54 | −24 | A | C |
| ATOM | 2361 | CB | VAL | A | 563 | 7.361 | 0.341 | 28.161 | 1.00 | 10.70 | | A | C |
| ANISOU | 2361 | CB | VAL | A | 563 | 1299 | 1395 | 1372 | 29 | 68 | 19 | A | C |
| ATOM | 2363 | CG1 | VAL | A | 563 | 8.574 | 0.604 | 29.051 | 1.00 | 11.76 | | A | C |
| ANISOU | 2363 | CG1 | VAL | A | 563 | 1401 | 1449 | 1616 | 31 | −27 | 0 | A | C |
| ATOM | 2367 | CG2 | VAL | A | 563 | 7.275 | −1.143 | 27.774 | 1.00 | 11.15 | | A | C |
| ANISOU | 2367 | CG2 | VAL | A | 563 | 1313 | 1418 | 1504 | −63 | 72 | 19 | A | C |
| ATOM | 2371 | C | VAL | A | 563 | 4.863 | 0.722 | 27.912 | 1.00 | 10.38 | | A | C |
| ANISOU | 2371 | C | VAL | A | 563 | 1292 | 1348 | 1303 | 42 | 89 | −65 | A | C |
| ATOM | 2372 | O | VAL | A | 563 | 4.009 | −0.146 | 28.064 | 1.00 | 11.15 | | A | O |
| ANISOU | 2372 | O | VAL | A | 563 | 1408 | 1398 | 1430 | −81 | 37 | −177 | A | O |
| ATOM | 2373 | N | LYS | A | 564 | 4.828 | 1.584 | 26.913 | 1.00 | 10.63 | | A | N |
| ANISOU | 2373 | N | LYS | A | 564 | 1296 | 1358 | 1384 | 95 | 76 | −23 | A | N |
| ATOM | 2375 | CA | LYS | A | 564 | 3.696 | 1.692 | 26.000 | 1.00 | 11.44 | | A | C |
| ANISOU | 2375 | CA | LYS | A | 564 | 1485 | 1469 | 1391 | 36 | −19 | −51 | A | C |
| ATOM | 2377 | CB | LYS | A | 564 | 2.766 | 2.828 | 26.410 | 1.00 | 13.11 | | A | C |
| ANISOU | 2377 | CB | LYS | A | 564 | 1649 | 1790 | 1540 | 137 | 56 | 26 | A | C |
| ATOM | 2380 | CG | LYS | A | 564 | 2.215 | 2.624 | 27.825 | 1.00 | 15.67 | | A | C |
| ANISOU | 2380 | CG | LYS | A | 564 | 2152 | 2218 | 1583 | 118 | −24 | −25 | A | C |
| ATOM | 2383 | CD | LYS | A | 564 | 0.846 | 3.066 | 28.038 | 1.00 | 17.90 | | A | C |
| ANISOU | 2383 | CD | LYS | A | 564 | 2303 | 2192 | 2305 | 8 | 24 | −23 | A | C |
| ATOM | 2386 | CE | LYS | A | 564 | 0.585 | 3.113 | 29.531 | 1.00 | 18.28 | | A | C |
| ANISOU | 2386 | CE | LYS | A | 564 | 2360 | 2253 | 2331 | 158 | 81 | −141 | A | C |
| ATOM | 2389 | NZ | LYS | A | 564 | −0.781 | 2.659 | 29.852 | 1.00 | 22.77 | | A | N |
| ANISOU | 2389 | NZ | LYS | A | 564 | 2792 | 3039 | 2819 | −210 | 121 | −24 | A | N |
| ATOM | 2393 | C | LYS | A | 564 | 4.172 | 1.938 | 24.591 | 1.00 | 10.77 | | A | C |
| ANISOU | 2393 | C | LYS | A | 564 | 1309 | 1375 | 1408 | 55 | 22 | −16 | A | C |
| ATOM | 2394 | O | LYS | A | 564 | 4.941 | 2.864 | 24.328 | 1.00 | 10.74 | | A | O |
| ANISOU | 2394 | O | LYS | A | 564 | 1239 | 1341 | 1500 | 120 | 52 | −122 | A | O |
| ATOM | 2395 | N | LEU | A | 565 | 3.730 | 1.079 | 23.689 | 1.00 | 10.25 | | A | N |
| ANISOU | 2395 | N | LEU | A | 565 | 1254 | 1252 | 1389 | 53 | 28 | −25 | A | N |
| ATOM | 2397 | CA | LEU | A | 565 | 4.035 | 1.227 | 22.285 | 1.00 | 10.59 | | A | C |
| ANISOU | 2397 | CA | LEU | A | 565 | 1364 | 1334 | 1322 | 36 | −44 | 11 | A | C |
| ATOM | 2399 | CB | LEU | A | 565 | 3.543 | −0.003 | 21.533 | 1.00 | 11.06 | | A | C |
| ANISOU | 2399 | CB | LEU | A | 565 | 1495 | 1274 | 1430 | 23 | −75 | 87 | A | C |
| ATOM | 2402 | CG | LEU | A | 565 | 3.798 | −0.071 | 20.038 | 1.00 | 12.33 | | A | C |
| ANISOU | 2402 | CG | LEU | A | 565 | 1649 | 1558 | 1476 | 100 | −72 | −66 | A | C |
| ATOM | 2404 | CD1 | LEU | A | 565 | 5.275 | −0.056 | 19.762 | 1.00 | 13.31 | | A | C |
| ANISOU | 2404 | CD1 | LEU | A | 565 | 1811 | 1742 | 1501 | 103 | 88 | −47 | A | C |
| ATOM | 2408 | CD2 | LEU | A | 565 | 3.139 | −1.293 | 19.470 | 1.00 | 13.66 | | A | C |
| ANISOU | 2408 | CD2 | LEU | A | 565 | 1947 | 1735 | 1509 | 56 | −141 | 58 | A | C |
| ATOM | 2412 | C | LEU | A | 565 | 3.378 | 2.475 | 21.718 | 1.00 | 11.22 | | A | C |
| ANISOU | 2412 | C | LEU | A | 565 | 1427 | 1366 | 1468 | 56 | 20 | −44 | A | C |
| ATOM | 2413 | O | LEU | A | 565 | 2.191 | 2.721 | 21.937 | 1.00 | 11.52 | | A | O |
| ANISOU | 2413 | O | LEU | A | 565 | 1372 | 1402 | 1603 | 214 | 113 | −16 | A | O |
| ATOM | 2414 | N | GLY | A | 566 | 4.140 | 3.232 | 20.942 | 1.00 | 12.46 | | A | N |
| ANISOU | 2414 | N | GLY | A | 566 | 1579 | 1574 | 1578 | 46 | −6 | −14 | A | N |
| ATOM | 2416 | CA | GLY | A | 566 | 3.628 | 4.441 | 20.315 | 1.00 | 13.36 | | A | C |
| ANISOU | 2416 | CA | GLY | A | 566 | 1744 | 1575 | 1756 | 50 | 45 | 14 | A | C |
| ATOM | 2419 | C | GLY | A | 566 | 2.830 | 4.241 | 19.038 | 1.00 | 15.83 | | A | C |
| ANISOU | 2419 | C | GLY | A | 566 | 1988 | 2012 | 2015 | 36 | 5 | −17 | A | C |
| ATOM | 2420 | O | GLY | A | 566 | 2.460 | 3.136 | 18.671 | 1.00 | 15.30 | | A | O |
| ANISOU | 2420 | O | GLY | A | 566 | 1818 | 1989 | 2003 | 230 | −5 | 24 | A | O |
| ATOM | 2421 | N | ASP | A | 567 | 2.610 | 5.366 | 18.358 | 1.00 | 18.88 | | A | N |
| ANISOU | 2421 | N | ASP | A | 567 | 2485 | 2299 | 2388 | 120 | −17 | 26 | A | N |
| ATOM | 2423 | CA | ASP | A | 567 | 1.821 | 5.454 | 17.123 | 1.00 | 22.18 | | A | C |
| ANISOU | 2423 | CA | ASP | A | 567 | 2820 | 2815 | 2790 | 34 | −77 | 10 | A | C |
| ATOM | 2425 | CB | ASP | A | 567 | 1.743 | 6.909 | 16.652 | 1.00 | 23.80 | | A | C |
| ANISOU | 2425 | CB | ASP | A | 567 | 3063 | 3001 | 2978 | 85 | −85 | 46 | A | C |
| ATOM | 2428 | CG | ASP | A | 567 | 1.123 | 7.810 | 17.647 | 1.00 | 27.77 | | A | C |
| ANISOU | 2428 | CG | ASP | A | 567 | 3596 | 3515 | 3436 | 94 | −1 | −93 | A | C |
| ATOM | 2429 | OD1 | ASP | A | 567 | 0.351 | 7.324 | 18.502 | 1.00 | 31.50 | | A | O |
| ANISOU | 2429 | OD1 | ASP | A | 567 | 3849 | 4126 | 3993 | −103 | 119 | 59 | A | O |
| ATOM | 2430 | OD2 | ASP | A | 567 | 1.370 | 9.031 | 17.647 | 1.00 | 32.32 | | A | O |
| ANISOU | 2430 | OD2 | ASP | A | 567 | 4227 | 3735 | 4317 | −132 | −17 | 8 | A | O |
| ATOM | 2431 | C | ASP | A | 567 | 2.407 | 4.717 | 15.954 | 1.00 | 23.94 | | A | C |
| ANISOU | 2431 | C | ASP | A | 567 | 3056 | 3019 | 3019 | 49 | 15 | 21 | A | C |
| ATOM | 2432 | O | ASP | A | 567 | 3.570 | 4.375 | 15.926 | 1.00 | 20.99 | | A | O |
| ANISOU | 2432 | O | ASP | A | 567 | 2719 | 2583 | 2671 | 42 | −104 | 189 | A | O |
| ATOM | 2433 | N | PHE | A | 568 | 1.581 | 4.572 | 14.934 | 1.00 | 27.78 | | A | N |
| ANISOU | 2433 | N | PHE | A | 568 | 3437 | 3614 | 3502 | 58 | −107 | 0 | A | N |
| ATOM | 2435 | CA | PHE | A | 568 | 1.940 | 3.852 | 13.718 | 1.00 | 31.07 | | A | C |
| ANISOU | 2435 | CA | PHE | A | 568 | 3939 | 3981 | 3883 | 45 | −33 | −57 | A | C |
| ATOM | 2437 | CB | PHE | A | 568 | 0.690 | 3.615 | 12.890 | 1.00 | 31.44 | | A | C |
| ANISOU | 2437 | CB | PHE | A | 568 | 4014 | 4000 | 3932 | 35 | −50 | −2 | A | C |
| ATOM | 2440 | CG | PHE | A | 568 | −0.310 | 2.813 | 13.602 | 1.00 | 32.10 | | A | C |
| ANISOU | 2440 | CG | PHE | A | 568 | 4049 | 4112 | 4033 | −49 | 8 | −29 | A | C |
| ATOM | 2441 | CD1 | PHE | A | 568 | −0.090 | 1.468 | 13.790 | 1.00 | 33.05 | | A | C |
| ANISOU | 2441 | CD1 | PHE | A | 568 | 4152 | 4222 | 4181 | 78 | 8 | 6 | A | C |
| ATOM | 2443 | CE1 | PHE | A | 568 | −0.992 | 0.699 | 14.489 | 1.00 | 33.55 | | A | C |
| ANISOU | 2443 | CE1 | PHE | A | 568 | 4179 | 4250 | 4319 | 4 | −26 | 53 | A | C |
| ATOM | 2445 | CZ | PHE | A | 568 | −2.118 | 1.290 | 15.038 | 1.00 | 33.51 | | A | C |
| ANISOU | 2445 | CZ | PHE | A | 568 | 4275 | 4211 | 4243 | 6 | 34 | 1 | A | C |
| ATOM | 2447 | CE2 | PHE | A | 568 | −2.335 | 2.650 | 14.866 | 1.00 | 33.37 | | A | C |
| ANISOU | 2447 | CE2 | PHE | A | 568 | 4194 | 4249 | 4234 | 6 | 6 | −25 | A | C |
| ATOM | 2449 | CD2 | PHE | A | 568 | −1.431 | 3.403 | 14.167 | 1.00 | 32.67 | | A | C |
| ANISOU | 2449 | CD2 | PHE | A | 568 | 4165 | 4077 | 4172 | 31 | 1 | −30 | A | C |
| ATOM | 2451 | C | PHE | A | 568 | 2.952 | 4.572 | 12.891 | 1.00 | 34.06 | | A | C |
| ANISOU | 2451 | C | PHE | A | 568 | 4336 | 4339 | 4266 | −22 | −41 | 24 | A | C |
| ATOM | 2452 | O | PHE | A | 568 | 3.100 | 5.787 | 12.998 | 1.00 | 34.68 | | A | O |
| ANISOU | 2452 | O | PHE | A | 568 | 4525 | 4338 | 4311 | 59 | −28 | −26 | A | O |
| ATOM | 2453 | N | GLY | A | 569 | 3.653 | 3.800 | 12.061 | 1.00 | 37.04 | | A | N |
| ANISOU | 2453 | N | GLY | A | 569 | 4697 | 4714 | 4661 | 48 | −34 | −63 | A | N |
| ATOM | 2455 | CA | GLY | A | 569 | 4.677 | 4.325 | 11.180 | 1.00 | 39.68 | | A | C |
| ANISOU | 2455 | CA | GLY | A | 569 | 5005 | 5045 | 5025 | −14 | 29 | 0 | A | C |
| ATOM | 2458 | C | GLY | A | 569 | 4.199 | 4.680 | 9.781 | 1.00 | 42.00 | | A | C |
| ANISOU | 2458 | C | GLY | A | 569 | 5343 | 5348 | 5265 | 12 | −8 | 9 | A | C |
| ATOM | 2459 | O | GLY | A | 569 | 5.017 | 5.059 | 8.933 | 1.00 | 42.55 | | A | O |
| ANISOU | 2459 | O | GLY | A | 569 | 5375 | 5396 | 5396 | −26 | 54 | 23 | A | O |
| ATOM | 2460 | N | LEU | A | 570 | 2.890 | 4.581 | 9.533 | 1.00 | 44.32 | | A | N |
| ANISOU | 2460 | N | LEU | A | 570 | 5557 | 5638 | 5644 | −8 | 2 | −7 | A | N |
| ATOM | 2462 | CA | LEU | A | 570 | 2.346 | 4.764 | 8.172 | 1.00 | 46.07 | | A | C |
| ANISOU | 2462 | CA | LEU | A | 570 | 5828 | 5864 | 5810 | 2 | −10 | 0 | A | C |
| ATOM | 2464 | CB | LEU | A | 570 | 0.892 | 4.250 | 8.042 | 1.00 | 46.30 | | A | C |
| ANISOU | 2464 | CB | LEU | A | 570 | 5844 | 5877 | 5871 | 0 | −20 | 0 | A | C |
| ATOM | 2467 | CG | LEU | A | 570 | −0.034 | 4.052 | 9.255 | 1.00 | 46.98 | | A | C |
| ANISOU | 2467 | CG | LEU | A | 570 | 5939 | 5964 | 5945 | 1 | 4 | 2 | A | C |
| ATOM | 2469 | CD1 | LEU | A | 570 | −0.765 | 5.343 | 9.594 | 1.00 | 47.51 | | A | C |
| ANISOU | 2469 | CD1 | LEU | A | 570 | 6012 | 6000 | 6039 | 10 | 5 | −15 | A | C |
| ATOM | 2473 | CD2 | LEU | A | 570 | −1.032 | 2.924 | 9.007 | 1.00 | 47.32 | | A | C |
| ANISOU | 2473 | CD2 | LEU | A | 570 | 5977 | 5970 | 6030 | −7 | 6 | −8 | A | C |
| ATOM | 2477 | C | LEU | A | 570 | 2.470 | 6.210 | 7.655 | 1.00 | 47.30 | | A | C |
| ANISOU | 2477 | C | LEU | A | 570 | 5999 | 5969 | 6004 | −5 | 0 | −1 | A | C |
| ATOM | 2478 | O | LEU | A | 570 | 2.024 | 7.158 | 8.300 | 1.00 | 47.60 | | A | O |
| ANISOU | 2478 | O | LEU | A | 570 | 6036 | 6001 | 6049 | 6 | 1 | −27 | A | O |
| ATOM | 2479 | N | SER | A | 571 | 3.064 | 6.343 | 6.467 | 1.00 | 48.73 | | A | N |
| ANISOU | 2479 | N | SER | A | 571 | 6175 | 6189 | 6151 | −14 | 9 | 6 | A | N |
| ATOM | 2481 | CA | SER | A | 571 | 3.477 | 7.633 | 5.893 | 1.00 | 49.54 | | A | C |
| ANISOU | 2481 | CA | SER | A | 571 | 6285 | 6262 | 6275 | −9 | 3 | 14 | A | C |
| ATOM | 2483 | CB | SER | A | 571 | 4.172 | 7.412 | 4.535 | 1.00 | 49.55 | | A | C |
| ANISOU | 2483 | CB | SER | A | 571 | 6270 | 6281 | 6276 | 3 | 4 | 18 | A | C |
| ATOM | 2486 | OG | SER | A | 571 | 5.577 | 7.560 | 4.643 | 1.00 | 49.55 | | A | O |
| ANISOU | 2486 | OG | SER | A | 571 | 6267 | 6278 | 6279 | −13 | 8 | 35 | A | O |
| ATOM | 2488 | C | SER | A | 571 | 2.342 | 8.637 | 5.707 | 1.00 | 50.18 | | A | C |
| ANISOU | 2488 | C | SER | A | 571 | 6344 | 6361 | 6360 | 12 | 6 | 0 | A | C |
| ATOM | 2489 | O | SER | A | 571 | 1.164 | 8.282 | 5.771 | 1.00 | 50.60 | | A | O |
| ANISOU | 2489 | O | SER | A | 571 | 6400 | 6405 | 6419 | −16 | 15 | −8 | A | O |
| ATOM | 2490 | N | ARG | A | 572 | 2.727 | 9.887 | 5.452 | 1.00 | 50.82 | | A | N |
| ANISOU | 2490 | N | ARG | A | 572 | 6440 | 6420 | 6447 | −5 | 6 | 0 | A | N |
| ATOM | 2492 | CA | ARG | A | 572 | 1.783 | 10.990 | 5.235 | 1.00 | 51.28 | | A | C |
| ANISOU | 2492 | CA | ARG | A | 572 | 6492 | 6494 | 6497 | 5 | 1 | 1 | A | C |
| ATOM | 2494 | CB | ARG | A | 572 | 1.830 | 11.971 | 6.419 | 1.00 | 51.64 | | A | C |
| ANISOU | 2494 | CB | ARG | A | 572 | 6547 | 6531 | 6542 | 6 | 7 | −11 | A | C |
| ATOM | 2497 | CG | ARG | A | 572 | 3.205 | 12.606 | 6.691 | 1.00 | 52.82 | | A | C |
| ANISOU | 2497 | CG | ARG | A | 572 | 6657 | 6714 | 6695 | −19 | −6 | −9 | A | C |
| ATOM | 2500 | CD | ARG | A | 572 | 3.285 | 14.116 | 6.426 | 1.00 | 54.29 | | A | C |
| ANISOU | 2500 | CD | ARG | A | 572 | 6915 | 6823 | 6886 | 5 | 5 | 10 | A | C |
| ATOM | 2503 | NE | ARG | A | 572 | 4.593 | 14.668 | 6.793 | 1.005 | 5.46 | | A | N |
| ANISOU | 2503 | NE | ARG | A | 572 | 6988 | 7037 | 7045 | −22 | −19 | −6 | A | N |
| ATOM | 2505 | CZ | ARG | A | 572 | 4.888 | 15.968 | 6.845 | 1.00 | 56.06 | | A | C |
| ANISOU | 2505 | CZ | ARG | A | 572 | 7109 | 7064 | 7125 | −8 | −2 | 5 | A | C |
| ATOM | 2506 | NH1 | ARG | A | 572 | 3.977 | 16.889 | 6.550 | 1.00 | 56.24 | | A | N |
| ANISOU | 2506 | NH1 | ARG | A | 572 | 7113 | 7117 | 7136 | 6 | −2 | 14 | A | N |
| ATOM | 2509 | NH2 | ARG | A | 572 | 6.112 | 16.351 | 7.195 | 1.00 | 56.51 | | A | N |
| ANISOU | 2509 | NH2 | ARG | A | 572 | 7136 | 7146 | 7188 | −5 | −10 | −12 | A | N |
| ATOM | 2512 | C | ARG | A | 572 | 2.055 | 11.723 | 3.912 | 1.00 | 51.09 | | A | C |
| ANISOU | 2512 | C | ARG | A | 572 | 6476 | 6470 | 6466 | 6 | 3 | −5 | A | C |
| ATOM | 2513 | O | ARG | A | 572 | 1.921 | 12.950 | 3.829 | 1.00 | 51.39 | | A | O |
| ANISOU | 2513 | O | ARG | A | 572 | 6504 | 6510 | 6509 | 10 | 2 | −17 | A | O |
| ATOM | 2514 | N | TYR | A | 573 | 2.419 | 10.960 | 2.879 | 1.00 | 50.77 | | A | N |
| ANISOU | 2514 | N | TYR | A | 573 | 6439 | 6431 | 6418 | 1 | −1 | 3 | A | N |
| ATOM | 2516 | CA | TYR | A | 573 | 2.760 | 11.514 | 1.563 | 1.00 | 50.38 | | A | C |
| ANISOU | 2516 | CA | TYR | A | 573 | 6391 | 6380 | 6369 | −2 | −8 | −5 | A | C |
| ATOM | 2518 | CB | TYR | A | 573 | 4.283 | 11.462 | 1.352 | 1.00 | 50.71 | | A | C |
| ANISOU | 2518 | CB | TYR | A | 573 | 6417 | 6430 | 6420 | 1 | −3 | −3 | A | C |
| ATOM | 2521 | CG | TYR | A | 573 | 4.830 | 12.554 | 0.449 | 1.00 | 51.77 | | A | C |
| ANISOU | 2521 | CG | TYR | A | 573 | 6570 | 6547 | 6551 | −15 | 5 | 22 | A | C |
| ATOM | 2522 | CD1 | TYR | A | 573 | 5.530 | 12.242 | −0.721 | 1.00 | 52.38 | | A | C |
| ANISOU | 2522 | CD1 | TYR | A | 573 | 6632 | 6648 | 6622 | 5 | 25 | 0 | A | C |
| ATOM | 2524 | CE1 | TYR | A | 573 | 6.028 | 13.246 | −1.552 | 1.00 | 52.54 | | A | C |
| ANISOU | 2524 | GE1 | TYR | A | 573 | 6645 | 6649 | 6668 | −11 | 15 | 10 | A | C |
| ATOM | 2526 | CZ | TYR | A | 573 | 5.830 | 14.577 | −1.214 | 1.00 | 52.84 | | A | C |
| ANISOU | 2526 | CZ | TYR | A | 573 | 6692 | 6687 | 6695 | −5 | 15 | −8 | A | C |
| ATOM | 2527 | OH | TYR | A | 573 | 6.316 | 15.580 | −2.026 | 1.00 | 52.93 | | A | O |
| ANISOU | 2527 | OH | TYR | A | 573 | 6703 | 6694 | 6713 | −15 | 15 | 3 | A | O |
| ATOM | 2529 | CE2 | TYR | A | 573 | 5.141 | 14.909 | −0.059 | 1.00 | 52.70 | | A | C |
| ANISOU | 2529 | CE2 | TYR | A | 573 | 6675 | 6668 | 6679 | −18 | 12 | 4 | A | C |
| ATOM | 2531 | CD2 | TYR | A | 573 | 4.649 | 13.900 | 0.765 | 1.00 | 52.48 | | A | C |
| ANISOU | 2531 | CD2 | TYR | A | 573 | 6667 | 6613 | 6658 | 6 | 2 | −12 | A | C |
| ATOM | 2533 | C | TYR | A | 573 | 2.034 | 10.761 | 0.433 | 1.00 | 49.50 | | A | C |
| ANISOU | 2533 | C | TYR | A | 573 | 6292 | 6261 | 6253 | 0 | −2 | 18 | A | C |
| ATOM | 2534 | O | TYR | A | 573 | 1.740 | 9.566 | 0.562 | 1.00 | 49.79 | | A | O |
| ANISOU | 2534 | O | TYR | A | 573 | 6333 | 6284 | 6301 | −18 | −1 | −4 | A | O |
| ATOM | 2535 | N | ILE | A | 574 | 1.751 | 11.472 | −0.665 | 1.00 | 48.15 | | A | N |
| ANISOU | 2535 | N | ILE | A | 574 | 6101 | 6097 | 6096 | −7 | −17 | −21 | A | N |
| ATOM | 2537 | CA | ILE | A | 574 | 1.032 | 10.906 | −1.820 | 1.00 | 46.82 | | A | C |
| ANISOU | 2537 | CA | ILE | A | 574 | 5931 | 5911 | 5946 | 12 | 11 | −6 | A | C |
| ATOM | 2539 | CB | ILE | A | 574 | 0.824 | 11.987 | −2.966 | 1.00 | 47.04 | | A | C |
| ANISOU | 2539 | CB | ILE | A | 574 | 5958 | 5953 | 5959 | −3 | 2 | −3 | A | C |
| ATOM | 2541 | CG1 | ILE | A | 574 | 0.115 | 11.378 | −4.188 | 1.00 | 47.21 | | A | C |
| ANISOU | 2541 | CG1 | ILE | A | 574 | 5979 | 5977 | 5980 | 4 | −9 | −6 | A | C |
| ATOM | 2544 | CD1 | ILE | A | 574 | −0.113 | 12.358 | −5.328 | 1.00 | 47.28 | | A | C |
| ANISOU | 2544 | CD1 | ILE | A | 574 | 5982 | 5982 | 6001 | 15 | 2 | 10 | A | C |
| ATOM | 2548 | CG2 | ILE | A | 574 | 2.144 | 12.663 | −3.400 | 1.00 | 47.22 | | A | C |
| ANISOU | 2548 | CG2 | ILE | A | 574 | 5967 | 5985 | 5987 | −4 | 11 | −8 | A | C |
| ATOM | 2552 | C | ILE | A | 574 | 1.659 | 9.607 | −2.367 | 1.00 | 45.25 | | A | C |
| ANISOU | 2552 | C | ILE | A | 574 | 5692 | 5756 | 5745 | −17 | 24 | 4 | A | C |
| ATOM | 2553 | O | ILE | A | 574 | 1.095 | 8.513 | −2.190 | 1.00 | 45.46 | | A | O |
| ANISOU | 2553 | O | ILE | A | 574 | 5743 | 5753 | 5777 | −15 | 14 | 3 | A | O |
| ATOM | 2554 | N | GLU | A | 575 | 2.832 | 9.717 | −2.989 | 1.00 | 42.99 | | A | N |
| ANISOU | 2554 | N | GLU | A | 575 | 5468 | 5415 | 5448 | 6 | 5 | −23 | A | N |
| ATOM | 2556 | CA | GLU | A | 575 | 3.425 | 8.595 | −3.722 | 1.00 | 40.89 | | A | C |
| ANISOU | 2556 | CA | GLU | A | 575 | 5189 | 5163 | 5184 | 0 | −5 | 26 | A | C |
| ATOM | 2558 | CB | GLU | A | 575 | 4.684 | 9.041 | −4.489 | 1.00 | 41.19 | | A | C |
| ANISOU | 2558 | CB | GLU | A | 575 | 5209 | 5210 | 5230 | 4 | 12 | 4 | A | C |
| ATOM | 2561 | CG | GLU | A | 575 | 4.416 | 9.455 | −5.931 | 1.00 | 42.08 | | A | C |
| ANISOU | 2561 | CG | GLU | A | 575 | 5364 | 5308 | 5315 | 6 | −12 | 5 | A | C |
| ATOM | 2564 | CD | GLU | A | 575 | 5.674 | 9.887 | −6.668 | 1.00 | 43.35 | | A | C |
| ANISOU | 2564 | CD | GLU | A | 575 | 5470 | 5494 | 5507 | −6 | 35 | 13 | A | C |
| ATOM | 2565 | OE1 | GLU | A | 575 | 6.746 | 9.274 | −6.448 | 1.00 | 43.91 | | A | O |
| ANISOU | 2565 | OE1 | GLU | A | 575 | 5551 | 5535 | 5596 | 35 | −28 | 40 | A | O |
| ATOM | 2566 | OE2 | GLU | A | 575 | 5.590 | 10.844 | −7.476 | 1.00 | 44.09 | | A | O |
| ANISOU | 2566 | OE2 | GLU | A | 575 | 5633 | 5563 | 5553 | 11 | −1 | 40 | A | O |
| ATOM | 2567 | C | GLU | A | 575 | 3.737 | 7.367 | −2.853 | 1.00 | 38.22 | | A | C |
| ANISOU | 2567 | C | GLU | A | 575 | 4849 | 4868 | 4803 | −3 | −7 | −34 | A | C |
| ATOM | 2568 | O | GLU | A | 575 | 3.833 | 6.259 | −3.397 | 1.00 | 38.54 | | A | O |
| ANISOU | 2568 | O | GLU | A | 575 | 4862 | 4870 | 4909 | 11 | −1 | −16 | A | O |
| ATOM | 2569 | N | ASP | A | 576 | 3.894 | 7.574 | −1.534 | 1.00 | 34.64 | | A | N |
| ANISOU | 2569 | N | ASP | A | 576 | 4378 | 4342 | 4440 | 16 | 15 | 34 | A | N |
| ATOM | 2571 | CA | ASP | A | 576 | 4.123 | 6.525 | −0.520 | 1.00 | 31.46 | | A | C |
| ANISOU | 2571 | CA | ASP | A | 576 | 3940 | 3975 | 4038 | 49 | 17 | −35 | A | C |
| ATOM | 2573 | CB | ASP | A | 576 | 2.798 | 5.899 | −0.068 | 1.00 | 31.90 | | A | C |
| ANISOU | 2573 | CB | ASP | A | 576 | 4039 | 3982 | 4099 | 11 | 21 | 18 | A | C |
| ATOM | 2576 | CG | ASP | A | 576 | 2.962 | 4.913 | 1.094 | 1.00 | 32.79 | | A | C |
| ANISOU | 2576 | CG | ASP | A | 576 | 4172 | 4136 | 4151 | 46 | 9 | 10 | A | C |
| ATOM | 2577 | OD1 | ASP | A | 576 | 3.073 | 3.705 | 0.828 | 1.00 | 30.61 | | A | O |
| ANISOU | 2577 | OD1 | ASP | A | 576 | 3905 | 3925 | 3798 | 24 | 40 | −7 | A | O |
| ATOM | 2578 | OD2 | ASP | A | 576 | 2.943 | 5.232 | 2.306 | 1.00 | 36.41 | | A | O |
| ANISOU | 2578 | OD2 | ASP | A | 576 | 4694 | 4671 | 4466 | 27 | −10 | −129 | A | O |
| ATOM | 2579 | C | ASP | A | 576 | 5.135 | 5.445 | −0.921 | 1.00 | 27.81 | | A | C |
| ANISOU | 2579 | C | ASP | A | 576 | 3527 | 3515 | 3523 | −2 | −36 | 26 | A | C |
| ATOM | 2580 | O | ASP | A | 576 | 5.102 | 4.917 | −2.026 | 1.00 | 26.13 | | A | O |
| ANISOU | 2580 | O | ASP | A | 576 | 3282 | 3194 | 3451 | 36 | −28 | 40 | A | O |
| ATOM | 2581 | N | GLU | A | 577 | 6.030 | 5.127 | 0.006 | 1.00 | 24.06 | | A | N |
| ANISOU | 2581 | N | GLU | A | 577 | 3046 | 3003 | 3090 | 24 | 47 | −22 | A | N |
| ATOM | 2583 | CA | GLU | A | 577 | 7.183 | 4.284 | −0.294 | 1.00 | 21.31 | | A | C |
| ANISOU | 2583 | CA | GLU | A | 577 | 2749 | 2641 | 2704 | −29 | −1 | 13 | A | C |
| ATOM | 2585 | CB | GLU | A | 577 | 8.290 | 4.492 | 0.743 | 1.00 | 21.05 | | A | C |
| ANISOU | 2585 | CB | GLU | A | 577 | 2716 | 2636 | 2645 | 8 | 30 | 15 | A | C |
| ATOM | 2588 | CG | GLU | A | 577 | 8.936 | 5.867 | 0.652 | 1.00 | 22.39 | | A | C |
| ANISOU | 2588 | CG | GLU | A | 577 | 2889 | 2763 | 2855 | −53 | −6 | −43 | A | C |
| ATOM | 2591 | CD | GLU | A | 577 | 10.323 | 5.932 | 1.267 | 1.00 | 23.68 | | A | C |
| ANISOU | 2591 | CD | GLU | A | 577 | 2991 | 3002 | 3004 | 31 | −25 | 42 | A | C |
| ATOM | 2592 | OE1 | GLU | A | 577 | 11.175 | 5.095 | 0.933 | 1.00 | 22.74 | | A | O |
| ANISOU | 2592 | OE1 | GLU | A | 577 | 2823 | 2940 | 2876 | −98 | 27 | 85 | A | O |
| ATOM | 2593 | OE2 | GLU | A | 577 | 10.557 | 6.839 | 2.088 | 1.00 | 26.39 | | A | O |
| ANISOU | 2593 | OE2 | GLU | A | 577 | 3441 | 3169 | 3418 | −40 | −106 | −30 | A | O |
| ATOM | 2594 | C | GLU | A | 577 | 6.844 | 2.799 | −0.430 | 1.00 | 19.18 | | A | C |
| ANISOU | 2594 | C | GLU | A | 577 | 2438 | 2448 | 2401 | −10 | 3 | 39 | A | C |
| ATOM | 2595 | O | GLU | A | 577 | 7.743 | 2.010 | −0.666 | 1.00 | 18.10 | | A | O |
| ANISOU | 2595 | O | GLU | A | 577 | 2407 | 2158 | 2312 | −54 | −14 | 67 | A | O |
| ATOM | 2596 | N | ASP | A | 578 | 5.564 | 2.425 | −0.324 | 1.00 | 17.39 | | A | N |
| ANISOU | 2596 | N | ASP | A | 578 | 2240 | 2225 | 2140 | 21 | −15 | 16 | A | N |
| ATOM | 2598 | CA | ASP | A | 578 | 5.142 | 1.064 | −0.671 | 1.00 | 16.41 | | A | C |
| ANISOU | 2598 | CA | ASP | A | 578 | 2082 | 2110 | 2040 | 10 | 0 | 22 | A | C |
| ATOM | 2600 | CB | ASP | A | 578 | 3.747 | 0.728 | −0.108 | 1.00 | 16.70 | | A | C |
| ANISOU | 2600 | CB | ASP | A | 578 | 2102 | 2185 | 2056 | 23 | −22 | 5 | A | C |
| ATOM | 2603 | CG | ASP | A | 578 | 3.721 | 0.575 | 1.396 | 1.00 | 17.80 | | A | C |
| ANISOU | 2603 | CG | ASP | A | 578 | 2269 | 2283 | 2210 | −25 | 4 | 73 | A | C |
| ATOM | 2604 | OD1 | ASP | A | 578 | 4.744 | 0.836 | 2.080 | 1.00 | 18.44 | | A | O |
| ANISOU | 2604 | OD1 | ASP | A | 578 | 2460 | 2283 | 2260 | 33 | −34 | 46 | A | O |
| ATOM | 2605 | OD2 | ASP | A | 578 | 2.673 | 0.168 | 1.958 | 1.00 | 19.32 | | A | O |
| ANISOU | 2605 | OD2 | ASP | A | 578 | 2556 | 2804 | 1980 | 25 | 129 | 191 | A | O |
| ATOM | 2606 | C | ASP | A | 578 | 5.101 | 0.850 | −2.194 | 1.00 | 15.80 | | A | C |
| ANISOU | 2606 | C | ASP | A | 578 | 2021 | 1999 | 1983 | 25 | 12 | 1 | A | C |
| ATOM | 2607 | O | ASP | A | 578 | 4.982 | −0.283 | −2.661 | 1.00 | 15.16 | | A | O |
| ANISOU | 2607 | O | ASP | A | 578 | 1934 | 2018 | 1806 | 66 | 29 | −29 | A | O |
| ATOM | 2608 | N | TYR | A | 579 | 5.154 | 1.942 | −2.947 | 1.00 | 15.54 | | A | N |
| ANISOU | 2608 | N | TYR | A | 579 | 1978 | 1989 | 1934 | −25 | 22 | 0 | A | N |
| ATOM | 2610 | CA | TYR | A | 579 | 4.949 | 1.919 | −4.396 | 1.00 | 15.86 | | A | C |
| ANISOU | 2610 | CA | TYR | A | 579 | 2010 | 2019 | 1994 | 16 | −14 | 20 | A | C |
| ATOM | 2612 | CB | TYR | A | 579 | 3.878 | 2.949 | −4.751 | 1.00 | 15.76 | | A | C |
| ANISOU | 2612 | CB | TYR | A | 579 | 2011 | 1982 | 1992 | 6 | 3 | 45 | A | C |
| ATOM | 2615 | CG | TYR | A | 579 | 2.527 | 2.607 | −4.227 | 1.00 | 16.35 | | A | C |
| ANISOU | 2615 | CG | TYR | A | 579 | 2024 | 2045 | 2141 | 67 | 1 | 17 | A | C |
| ATOM | 2616 | CD1 | TYR | A | 579 | 1.619 | 1.912 | −5.010 | 1.00 | 17.24 | | A | C |
| ANISOU | 2616 | CD1 | TYR | A | 579 | 2203 | 2167 | 2179 | 47 | −51 | −9 | A | C |
| ATOM | 2618 | CE1 | TYR | A | 579 | 0.367 | 1.573 | −4.526 | 1.00 | 18.73 | | A | C |
| ANISOU | 2618 | CE1 | TYR | A | 579 | 2353 | 2364 | 2399 | −10 | −14 | 23 | A | C |
| ATOM | 2620 | CZ | TYR | A | 579 | −0.002 | 1.946 | −3.259 | 1.00 | 19.96 | | A | C |
| ANISOU | 2620 | CZ | TYR | A | 579 | 2495 | 2490 | 2596 | 45 | 50 | −42 | A | C |
| ATOM | 2621 | OH | TYR | A | 579 | −1.257 | 1.594 | −2.806 | 1.00 | 23.30 | | A | O |
| ANISOU | 2621 | OH | TYR | A | 579 | 2638 | 3008 | 3204 | −9 | 164 | −76 | A | O |
| ATOM | 2623 | CE2 | TYR | A | 579 | 0.876 | 2.636 | −2.453 | 1.00 | 19.53 | | A | C |
| ANISOU | 2623 | CE2 | TYR | A | 579 | 2459 | 2491 | 2469 | 42 | 95 | −66 | A | C |
| ATOM | 2625 | CD2 | TYR | A | 579 | 2.149 | 2.962 | −2.936 | 1.00 | 18.93 | | A | C |
| ANISOU | 2625 | CD2 | TYR | A | 579 | 2448 | 2411 | 2333 | −3 | 12 | −65 | A | C |
| ATOM | 2627 | C | TYR | A | 579 | 6.214 | 2.199 | −5.206 | 1.00 | 16.65 | | A | C |
| ANISOU | 2627 | C | TYR | A | 579 | 2123 | 2109 | 2091 | 30 | 8 | 2 | A | C |
| ATOM | 2628 | O | TYR | A | 579 | 6.240 | 1.995 | −6.419 | 1.00 | 17.08 | | A | O |
| ANISOU | 2628 | O | TYR | A | 579 | 2170 | 2171 | 2148 | 4 | 7 | 12 | A | O |
| ATOM | 2629 | N | TYR | A | 580 | 7.257 | 2.676 | −4.538 | 1.00 | 17.48 | | A | N |
| ANISOU | 2629 | N | TYR | A | 580 | 2180 | 2208 | 2254 | 12 | 13 | −20 | A | N |
| ATOM | 2631 | CA | TYR | A | 580 | 8.512 | 3.024 | −5.197 | 1.00 | 17.86 | | A | C |
| ANISOU | 2631 | CA | TYR | A | 580 | 2242 | 2256 | 2285 | −1 | 37 | −5 | A | C |
| ATOM | 2633 | CB | TYR | A | 580 | 8.451 | 4.465 | −5.766 | 1.00 | 18.09 | | A | C |
| ANISOU | 2633 | CB | TYR | A | 580 | 2274 | 2296 | 2302 | −8 | 22 | 35 | A | C |
| ATOM | 2636 | CG | TYR | A | 580 | 8.481 | 5.556 | −4.709 | 1.00 | 18.48 | | A | C |
| ANISOU | 2636 | CG | TYR | A | 580 | 2326 | 2301 | 2392 | −21 | 15 | 0 | A | C |
| ATOM | 2637 | CD1 | TYR | A | 580 | 9.689 | 6.102 | −4.268 | 1.00 | 17.63 | | A | C |
| ANISOU | 2637 | CD1 | TYR | A | 580 | 2232 | 2214 | 2252 | 18 | 33 | 99 | A | C |
| ATOM | 2639 | CE1 | TYR | A | 580 | 9.729 | 7.072 | −3.300 | 1.00 | 18.28 | | A | C |
| ANISOU | 2639 | CE1 | TYR | A | 580 | 2343 | 2148 | 2453 | −45 | 47 | 77 | A | C |
| ATOM | 2641 | CZ | TYR | A | 580 | 8.549 | 7.547 | −2.745 | 1.00 | 19.74 | | A | C |
| ANISOU | 2641 | CZ | TYR | A | 580 | 2440 | 2514 | 2544 | −11 | 20 | 51 | A | C |
| ATOM | 2642 | OH | TYR | A | 580 | 8.594 | 8.526 | −1.776 | 1.00 | 21.33 | | A | O |
| ANISOU | 2642 | OH | TYR | A | 580 | 2750 | 2415 | 2937 | −128 | 50 | 68 | A | O |
| ATOM | 2644 | CE2 | TYR | A | 580 | 7.326 | 7.024 | −3.158 | 1.00 | 20.10 | | A | C |
| ANISOU | 2644 | CE2 | TYR | A | 580 | 2490 | 2505 | 2639 | −21 | 38 | −24 | A | C |
| ATOM | 2646 | CD2 | TYR | A | 580 | 7.300 | 6.041 | −4.143 | 1.00 | 18.84 | | A | C |
| ANISOU | 2646 | CD2 | TYR | A | 580 | 2334 | 2379 | 2446 | −16 | 19 | 40 | A | C |
| ATOM | 2648 | C | TYR | A | 580 | 9.713 | 2.848 | −4.271 | 1.00 | 17.89 | | A | C |
| ANISOU | 2648 | C | TYR | A | 580 | 2269 | 2274 | 2253 | −32 | 32 | −16 | A | C |
| ATOM | 2649 | O | TYR | A | 580 | 9.600 | 2.942 | −3.030 | 1.00 | 17.99 | | A | O |
| ANISOU | 2649 | O | TYR | A | 580 | 2292 | 2255 | 2288 | −54 | 105 | 5 | A | O |
| ATOM | 2650 | N | LYS | A | 581 | 10.863 | 2.584 | −4.893 | 1.00 | 18.02 | | A | N |
| ANISOU | 2650 | N | LYS | A | 581 | 2255 | 2257 | 2333 | 4 | 1 | 0 | A | N |
| ATOM | 2652 | CA | LYS | A | 581 | 12.136 | 2.505 | −4.201 | 1.00 | 17.79 | | A | C |
| ANISOU | 2652 | CA | LYS | A | 581 | 2238 | 2225 | 2297 | −19 | 13 | −19 | A | C |
| ATOM | 2654 | CB | LYS | A | 581 | 13.032 | 1.431 | −4.820 | 1.00 | 18.41 | | A | C |
| ANISOU | 2654 | CB | LYS | A | 581 | 2290 | 2298 | 2406 | 13 | 6 | −25 | A | C |
| ATOM | 2657 | CG | LYS | A | 581 | 12.516 | 0.033 | −4.640 | 1.00 | 19.43 | | A | C |
| ANISOU | 2657 | CG | LYS | A | 581 | 2453 | 2376 | 2553 | −12 | 7 | −17 | A | C |
| ATOM | 2660 | CD | LYS | A | 581 | 12.368 | −0.307 | −3.177 | 1.00 | 20.34 | | A | C |
| ANISOU | 2660 | CD | LYS | A | 581 | 2563 | 2558 | 2605 | 14 | −29 | 1 | A | C |
| ATOM | 2663 | CE | LYS | A | 581 | 12.436 | −1.810 | −2.931 | 1.00 | 20.07 | | A | C |
| ANISOU | 2663 | CE | LYS | A | 581 | 2521 | 2484 | 2619 | −2 | 14 | −8 | A | C |
| ATOM | 2666 | NZ | LYS | A | 581 | 13.759 | −2.435 | −3.299 | 1.00 | 21.13 | | A | N |
| ANISOU | 2666 | NZ | LYS | A | 581 | 2474 | 2729 | 2823 | −12 | 42 | −38 | A | N |
| ATOM | 2670 | C | LYS | A | 581 | 12.818 | 3.853 | −4.316 | 1.00 | 16.99 | | A | C |
| ANISOU | 2670 | C | LYS | A | 581 | 2141 | 2142 | 2172 | −8 | 36 | 30 | A | C |
| ATOM | 2671 | O | LYS | A | 581 | 13.245 | 4.263 | −5.413 | 1.00 | 18.03 | | A | O |
| ANISOU | 2671 | O | LYS | A | 581 | 2308 | 2234 | 2309 | 25 | 84 | 38 | A | O |
| ATOM | 2672 | N | ALA | A | 582 | 12.902 | 4.536 | −3.183 | 1.00 | 15.82 | | A | N |
| ANISOU | 2672 | N | ALA | A | 582 | 1955 | 1993 | 2060 | −33 | 45 | 46 | A | N |
| ATOM | 2674 | CA | ALA | A | 582 | 13.586 | 5.810 | −3.082 | 1.00 | 15.68 | | A | C |
| ANISOU | 2674 | CA | ALA | A | 582 | 1978 | 1922 | 2058 | 0 | 23 | 38 | A | C |
| ATOM | 2676 | CB | ALA | A | 582 | 13.321 | 6.444 | −1.738 | 1.00 | 15.15 | | A | C |
| ANISOU | 2676 | CB | ALA | A | 582 | 1912 | 1910 | 1931 | −7 | 8 | 103 | A | C |
| ATOM | 2680 | C | ALA | A | 582 | 15.083 | 5.632 | −3.284 | 1.00 | 16.35 | | A | C |
| ANISOU | 2680 | C | ALA | A | 582 | 2012 | 1998 | 2201 | 8 | 22 | 50 | A | C |
| ATOM | 2681 | O | ALA | A | 582 | 15.623 | 4.587 | −2.984 | 1.00 | 16.94 | | A | O |
| ANISOU | 2681 | O | ALA | A | 582 | 2032 | 1912 | 2491 | 11 | 50 | 104 | A | O |
| ATOM | 2682 | N | SER | A | 583 | 15.753 | 6.661 | −3.796 | 1.00 | 16.37 | | A | N |
| ANISOU | 2682 | N | SER | A | 583 | 2060 | 2003 | 2157 | 0 | 39 | 73 | A | N |
| ATOM | 2684 | CA | SER | A | 583 | 17.217 | 6.677 | −3.778 | 1.00 | 16.93 | | A | C |
| ANISOU | 2684 | CA | SER | A | 583 | 2104 | 2085 | 2240 | 23 | 4 | 34 | A | C |
| ATOM | 2686 | CB | SER | A | 583 | 17.775 | 7.902 | −4.502 | 1.00 | 16.51 | | A | C |
| ANISOU | 2686 | CB | SER | A | 583 | 2019 | 2086 | 2168 | −2 | 44 | 4 | A | C |
| ATOM | 2689 | OG | SER | A | 583 | 17.566 | 7.822 | −5.897 | 1.00 | 14.11 | | A | O |
| ANISOU | 2689 | OG | SER | A | 583 | 1605 | 1680 | 2076 | 52 | −26 | −26 | A | O |
| ATOM | 2691 | C | SER | A | 583 | 17.735 | 6.681 | −2.346 | 1.00 | 17.70 | | A | C |
| ANISOU | 2691 | C | SER | A | 583 | 2243 | 2193 | 2287 | 10 | 10 | 67 | A | C |
| ATOM | 2692 | O | SER | A | 583 | 18.697 | 5.982 | −2.032 | 1.00 | 18.66 | | A | O |
| ANISOU | 2692 | O | SER | A | 583 | 2279 | 2288 | 2523 | 70 | −14 | 112 | A | O |
| ATOM | 2693 | N | VAL | A | 584 | 17.120 | 7.501 | −1.498 | 1.00 | 18.19 | | A | N |
| ANISOU | 2693 | N | VAL | A | 584 | 2287 | 2261 | 2361 | −5 | 29 | 56 | A | N |
| ATOM | 2695 | CA | VAL | A | 584 | 17.413 | 7.556 | −0.072 | 1.00 | 19.05 | | A | C |
| ANISOU | 2695 | CA | VAL | A | 584 | 2409 | 2408 | 2420 | −25 | 29 | 40 | A | C |
| ATOM | 2697 | CB | VAL | A | 584 | 18.096 | 8.871 | 0.357 | 1.00 | 19.66 | | A | C |
| ANISOU | 2697 | CB | VAL | A | 584 | 2511 | 2473 | 2485 | −4 | −4 | 10 | A | C |
| ATOM | 2699 | CG1 | VAL | A | 584 | 18.341 | 8.890 | 1.867 | 1.00 | 20.62 | | A | C |
| ANISOU | 2699 | CG1 | VAL | A | 584 | 2665 | 2634 | 2532 | −7 | −23 | −5 | A | C |
| ATOM | 2703 | CG2 | VAL | A | 584 | 19.381 | 9.060 | −0.379 | 1.00 | 20.53 | | A | C |
| ANISOU | 2703 | CG2 | VAL | A | 584 | 2621 | 2609 | 2567 | −47 | 31 | 11 | A | C |
| ATOM | 2707 | C | VAL | A | 584 | 16.078 | 7.449 | 0.648 | 1.00 | 18.95 | | A | C |
| ANISOU | 2707 | C | VAL | A | 584 | 2381 | 2398 | 2418 | −23 | 59 | 51 | A | C |
| ATOM | 2708 | O | VAL | A | 584 | 15.291 | 8.391 | 0.672 | 1.00 | 17.84 | | A | O |
| ANISOU | 2708 | O | VAL | A | 584 | 2262 | 2271 | 2245 | −157 | 161 | 219 | A | O |
| ATOM | 2709 | N | THR | A | 585 | 15.818 | 6.283 | 1.216 | 1.00 | 19.63 | | A | N |
| ANISOU | 2709 | N | THR | A | 585 | 2483 | 2451 | 2523 | 0 | 34 | 58 | A | N |
| ATOM | 2711 | CA | THR | A | 585 | 14.560 | 6.052 | 1.900 | 1.00 | 20.45 | | A | C |
| ANISOU | 2711 | CA | THR | A | 585 | 2574 | 2605 | 2590 | −10 | 24 | 71 | A | C |
| ATOM | 2713 | CB | THR | A | 585 | 14.287 | 4.549 | 2.007 | 1.00 | 21.03 | | A | C |
| ANISOU | 2713 | CB | THR | A | 585 | 2630 | 2664 | 2696 | 0 | −24 | 12 | A | C |
| ATOM | 2715 | OG1 | THR | A | 585 | 12.964 | 4.321 | 2.525 | 1.00 | 21.93 | | A | O |
| ANISOU | 2715 | OG1 | THR | A | 585 | 2848 | 2884 | 2600 | −100 | 150 | 177 | A | O |
| ATOM | 2717 | CG2 | THR | A | 585 | 15.209 | 3.897 | 3.026 | 1.00 | 21.35 | | A | C |
| ANISOU | 2717 | CG2 | THR | A | 585 | 2765 | 2668 | 2676 | 7 | −40 | 96 | A | C |
| ATOM | 2721 | C | THR | A | 585 | 14.512 | 6.709 | 3.281 | 1.00 | 20.98 | | A | C |
| ANISOU | 2721 | C | THR | A | 585 | 2671 | 2624 | 2676 | −26 | 17 | 66 | A | C |
| ATOM | 2722 | O | THR | A | 585 | 15.531 | 6.921 | 3.928 | 1.00 | 21.27 | | A | O |
| ANISOU | 2722 | O | THR | A | 585 | 2738 | 2558 | 2785 | −153 | 35 | 137 | A | O |
| ATOM | 2723 | N | ARG | A | 586 | 13.289 | 6.983 | 3.732 | 1.00 | 21.82 | | A | N |
| ANISOU | 2723 | N | ARG | A | 586 | 2782 | 2761 | 2745 | 31 | 47 | 43 | A | N |
| ATOM | 2725 | CA | ARG | A | 586 | 13.031 | 7.549 | 5.052 | 1.00 | 21.86 | | A | C |
| ANISOU | 2725 | CA | ARG | A | 586 | 2852 | 2719 | 2735 | −10 | 58 | 63 | A | C |
| ATOM | 2727 | CB | ARG | A | 586 | 11.813 | 8.473 | 4.979 | 1.00 | 23.02 | | A | C |
| ANISOU | 2727 | CB | ARG | A | 586 | 2935 | 2927 | 2883 | 4 | 38 | 5 | A | C |
| ATOM | 2730 | CG | ARG | A | 586 | 11.881 | 9.569 | 3.912 | 1.00 | 26.16 | | A | C |
| ANISOU | 2730 | CG | ARG | A | 586 | 3295 | 3342 | 3302 | −3 | 15 | 126 | A | C |
| ATOM | 2733 | CD | ARG | A | 586 | 11.676 | 10.988 | 4.464 | 1.00 | 30.42 | | A | C |
| ANISOU | 2733 | CD | ARG | A | 586 | 3912 | 3704 | 3939 | 10 | −21 | −49 | A | C |
| ATOM | 2736 | NE | ARG | A | 586 | 10.804 | 11.017 | 5.645 | 1.00 | 33.93 | | A | N |
| ANISOU | 2736 | NE | ARG | A | 586 | 4286 | 4331 | 4274 | 6 | 80 | 35 | A | N |
| ATOM | 2738 | CZ | ARG | A | 586 | 10.848 | 11.935 | 6.613 | 1.00 | 36.28 | | A | C |
| ANISOU | 2738 | CZ | ARG | A | 586 | 4628 | 4593 | 4562 | 5 | −4 | −70 | A | C |
| ATOM | 2739 | NH1 | ARG | A | 586 | 11.701 | 12.953 | 6.563 | 1.00 | 37.52 | | A | N |
| ANISOU | 2739 | NH1 | ARG | A | 586 | 4744 | 4718 | 4792 | −25 | 8 | −2 | A | N |
| ATOM | 2742 | NH2 | ARG | A | 586 | 10.010 | 11.845 | 7.639 | 1.00 | 37.48 | | A | N |
| ANISOU | 2742 | NH2 | ARG | A | 586 | 4743 | 4840 | 4655 | −18 | 32 | 2 | A | N |
| ATOM | 2745 | C | ARG | A | 586 | 12.743 | 6.405 | 6.040 | 1.00 | 20.86 | | A | C |
| ANISOU | 2745 | C | ARG | A | 586 | 2704 | 2585 | 2636 | −11 | 41 | 41 | A | C |
| ATOM | 2746 | O | ARG | A | 586 | 12.667 | 6.611 | 7.255 | 1.00 | 21.59 | | A | O |
| ANISOU | 2746 | O | ARG | A | 586 | 2933 | 2538 | 2732 | 11 | 112 | 56 | A | O |
| ATOM | 2747 | N | LEU | A | 587 | 12.581 | 5.215 | 5.483 | 1.00 | 18.66 | | A | N |
| ANISOU | 2747 | N | LEU | A | 587 | 2390 | 2310 | 2388 | 8 | 81 | 121 | A | N |
| ATOM | 2749 | CA | LEU | A | 587 | 12.161 | 4.031 | 6.232 | 1.00 | 17.34 | | A | C |
| ANISOU | 2749 | CA | LEU | A | 587 | 2176 | 2186 | 2226 | −5 | 40 | 118 | A | C |
| ATOM | 2751 | CB | LEU | A | 587 | 11.754 | 2.929 | 5.269 | 1.00 | 17.67 | | A | C |
| ANISOU | 2751 | CB | LEU | A | 587 | 2233 | 2283 | 2195 | 35 | 75 | 82 | A | C |
| ATOM | 2754 | CG | LEU | A | 587 | 10.543 | 3.200 | 4.382 | 1.00 | 19.24 | | A | C |
| ANISOU | 2754 | CG | LEU | A | 587 | 2414 | 2472 | 2422 | −25 | −27 | 102 | A | C |
| ATOM | 2756 | CD1 | LEU | A | 587 | 10.419 | 2.131 | 3.317 | 1.00 | 21.02 | | A | C |
| ANISOU | 2756 | CD1 | LEU | A | 587 | 2726 | 2638 | 2621 | 61 | −25 | 59 | A | C |
| ATOM | 2760 | CD2 | LEU | A | 587 | 9.293 | 3.265 | 5.247 | 1.00 | 21.78 | | A | C |
| ANISOU | 2760 | CD2 | LEU | A | 587 | 2677 | 2875 | 2721 | 26 | 34 | −31 | A | C |
| ATOM | 2764 | C | LEU | A | 587 | 13.279 | 3.522 | 7.136 | 1.00 | 15.98 | | A | C |
| ANISOU | 2764 | C | LEU | A | 587 | 1981 | 1985 | 2104 | 8 | 53 | 106 | A | C |
| ATOM | 2765 | O | LEU | A | 587 | 14.474 | 3.747 | 6.860 | 1.00 | 15.60 | | A | O |
| ANISOU | 2765 | O | LEU | A | 587 | 1918 | 1860 | 2148 | 24 | 18 | 215 | A | O |
| ATOM | 2766 | N | PRO | A | 588 | 12.896 | 2.852 | 8.224 | 1.00 | 14.09 | | A | N |
| ANISOU | 2766 | N | PRO | A | 588 | 1690 | 1800 | 1864 | 22 | −23 | 133 | A | N |
| ATOM | 2767 | CA | PRO | A | 588 | 13.861 | 2.313 | 9.194 | 1.00 | 13.50 | | A | C |
| ANISOU | 2767 | CA | PRO | A | 588 | 1699 | 1649 | 1781 | 24 | −50 | 41 | A | C |
| ATOM | 2769 | CB | PRO | A | 588 | 13.008 | 2.103 | 10.439 | 1.00 | 13.35 | | A | C |
| ANISOU | 2769 | CB | PRO | A | 588 | 1764 | 1590 | 1716 | 76 | −62 | −6 | A | C |
| ATOM | 2772 | CG | PRO | A | 588 | 11.673 | 1.809 | 9.928 | 1.00 | 13.43 | | A | C |
| ANISOU | 2772 | CG | PRO | A | 588 | 1667 | 1761 | 1672 | −40 | 50 | 68 | A | C |
| ATOM | 2775 | CD | PRO | A | 588 | 11.504 | 2.628 | 8.662 | 1.00 | 13.91 | | A | C |
| ANISOU | 2775 | CD | PRO | A | 588 | 1743 | 1700 | 1839 | 16 | 29 | 104 | A | C |
| ATOM | 2778 | C | PRO | A | 588 | 14.538 | 1.017 | 8.741 | 1.00 | 11.57 | | A | C |
| ANISOU | 2778 | C | PRO | A | 588 | 1422 | 1491 | 1483 | 21 | −49 | 5 | A | C |
| ATOM | 2779 | O | PRO | A | 588 | 14.303 | −0.065 | 9.268 | 1.00 | 11.14 | | A | O |
| ANISOU | 2779 | O | PRO | A | 588 | 1294 | 1574 | 1361 | 81 | −88 | 96 | A | O |
| ATOM | 2780 | N | ILE | A | 589 | 15.387 | 1.146 | 7.738 | 1.00 | 10.87 | | A | N |
| ANISOU | 2780 | N | ILE | A | 589 | 1413 | 1343 | 1373 | −5 | −84 | 38 | A | N |
| ATOM | 2782 | CA | ILE | A | 589 | 15.941 | 0.000 | 7.051 | 1.00 | 10.49 | | A | C |
| ANISOU | 2782 | CA | ILE | A | 589 | 1291 | 1358 | 1337 | 16 | −90 | 13 | A | C |
| ATOM | 2784 | CB | ILE | A | 589 | 16.956 | 0.492 | 5.983 | 1.00 | 11.11 | | A | C |
| ANISOU | 2784 | CB | ILE | A | 589 | 1437 | 1403 | 1379 | −34 | −57 | 12 | A | C |
| ATOM | 2786 | CG1 | ILE | A | 589 | 16.248 | 1.248 | 4.851 | 1.00 | 12.77 | | A | C |
| ANISOU | 2786 | CG1 | ILE | A | 589 | 1631 | 1555 | 1664 | 79 | −43 | 70 | A | C |
| ATOM | 2789 | CD1 | ILE | A | 589 | 15.267 | 0.388 | 4.044 | 1.00 | 13.65 | | A | C |
| ANISOU | 2789 | CD1 | ILE | A | 589 | 1905 | 1597 | 1681 | −16 | −40 | 94 | A | C |
| ATOM | 2793 | CG2 | ILE | A | 589 | 17.767 | −0.643 | 5.436 | 1.00 | 10.86 | | A | C |
| ANISOU | 2793 | CG2 | ILE | A | 589 | 1447 | 1421 | 1254 | −71 | 5 | −27 | A | C |
| ATOM | 2797 | C | ILE | A | 589 | 16.622 | −0.957 | 8.030 | 1.00 | 10.24 | | A | C |
| ANISOU | 2797 | C | ILE | A | 589 | 1371 | 1259 | 1262 | −26 | −33 | 49 | A | C |
| ATOM | 2798 | O | ILE | A | 589 | 16.470 | −2.163 | 7.911 | 1.00 | 9.58 | | A | O |
| ANISOU | 2798 | O | ILE | A | 589 | 1292 | 1125 | 1222 | 67 | −143 | 57 | A | O |
| ATOM | 2799 | N | LYS | A | 590 | 17.377 | −0.415 | 8.987 | 1.00 | 10.07 | | A | N |
| ANISOU | 2799 | N | LYS | A | 590 | 1285 | 1253 | 1286 | −3 | −20 | 82 | A | N |
| ATOM | 2801 | CA | LYS | A | 590 | 18.118 | −1.241 | 9.958 | 1.00 | 10.34 | | A | C |
| ANISOU | 2801 | CA | LYS | A | 590 | 1334 | 1280 | 1312 | 33 | −57 | 18 | A | C |
| ATOM | 2803 | CB | LYS | A | 590 | 19.118 | −0.367 | 10.716 | 1.00 | 9.88 | | A | C |
| ANISOU | 2803 | CB | LYS | A | 590 | 1334 | 1175 | 1244 | 26 | −69 | 104 | A | C |
| ATOM | 2806 | CG | LYS | A | 590 | 20.262 | 0.073 | 9.834 | 1.00 | 10.30 | | A | C |
| ANISOU | 2806 | CG | LYS | A | 590 | 1415 | 1305 | 1193 | 36 | 0 | 39 | A | C |
| ATOM | 2809 | CD | LYS | A | 590 | 21.127 | 1.144 | 10.494 | 1.00 | 11.73 | | A | C |
| ANISOU | 2809 | CD | LYS | A | 590 | 1555 | 1358 | 1542 | −46 | −207 | 182 | A | C |
| ATOM | 2812 | CE | LYS | A | 590 | 22.293 | 1.503 | 9.623 | 1.00 | 12.78 | | A | C |
| ANISOU | 2812 | CE | LYS | A | 590 | 1713 | 1558 | 1583 | −12 | −118 | 48 | A | C |
| ATOM | 2815 | NZ | LYS | A | 590 | 23.216 | 2.545 | 10.173 | 1.00 | 14.55 | | A | N |
| ANISOU | 2815 | NZ | LYS | A | 590 | 1950 | 1654 | 1923 | −89 | −189 | −17 | A | N |
| ATOM | 2819 | C | LYS | A | 590 | 17.232 | −2.059 | 10.923 | 1.00 | 9.74 | | A | C |
| ANISOU | 2819 | C | LYS | A | 590 | 1295 | 1165 | 1241 | 41 | −59 | 24 | A | C |
| ATOM | 2820 | O | LYS | A | 590 | 17.722 | −2.929 | 11.657 | 1.00 | 9.82 | | A | O |
| ANISOU | 2820 | O | LYS | A | 590 | 1207 | 1237 | 1287 | 92 | −113 | 118 | A | O |
| ATOM | 2821 | N | TRP | A | 591 | 15.938 | −1.740 | 10.960 | 1.00 | 9.17 | | A | N |
| ANISOU | 2821 | N | TRP | A | 591 | 1224 | 1156 | 1104 | 74 | 17 | 34 | A | N |
| ATOM | 2823 | CA | TRP | A | 591 | 14.938 | −2.432 | 11.754 | 1.00 | 9.07 | | A | C |
| ANISOU | 2823 | CA | TRP | A | 591 | 1188 | 1195 | 1061 | 10 | 10 | 20 | A | C |
| ATOM | 2825 | CB | TRP | A | 591 | 14.051 | −1.404 | 12.442 | 1.00 | 10.28 | | A | C |
| ANISOU | 2825 | CB | TRP | A | 591 | 1322 | 1298 | 1283 | 49 | −32 | −57 | A | C |
| ATOM | 2828 | CG | TRP | A | 591 | 14.679 | −0.623 | 13.537 | 1.00 | 10.96 | | A | C |
| ANISOU | 2828 | CG | TRP | A | 591 | 1478 | 1422 | 1263 | 66 | −57 | −46 | A | C |
| ATOM | 2829 | CD1 | TRP | A | 591 | 14.475 | −0.795 | 14.883 | 1.00 | 12.24 | | A | C |
| ANISOU | 2829 | CD1 | TRP | A | 591 | 1628 | 1561 | 1461 | 164 | −9 | −71 | A | C |
| ATOM | 2831 | NE1 | TRP | A | 591 | 15.162 | 0.164 | 15.582 | 1.00 | 13.31 | | A | N |
| ANISOU | 2831 | NE1 | TRP | A | 591 | 1741 | 1547 | 1768 | 249 | −62 | −150 | A | N |
| ATOM | 2833 | CE2 | TRP | A | 591 | 15.841 | 0.977 | 14.719 | 1.00 | 12.60 | | A | C |
| ANISOU | 2833 | CE2 | TRP | A | 591 | 1678 | 1550 | 1558 | 214 | −99 | −57 | A | C |
| ATOM | 2834 | CD2 | TRP | A | 591 | 15.525 | 0.538 | 13.408 | 1.00 | 10.95 | | A | C |
| ANISOU | 2834 | CD2 | TRP | A | 591 | 1348 | 1395 | 1416 | 212 | −69 | −162 | A | C |
| ATOM | 2835 | CE3 | TRP | A | 591 | 16.075 | 1.228 | 12.328 | 1.00 | 13.22 | | A | C |
| ANISOU | 2835 | CE3 | TRP | A | 591 | 1656 | 1554 | 1810 | 37 | −31 | 14 | A | C |
| ATOM | 2837 | CZ3 | TRP | A | 591 | 16.897 | 2.342 | 12.567 | 1.00 | 14.15 | | A | C |
| ANISOU | 2837 | CZ3 | TRP | A | 591 | 1859 | 1677 | 1837 | 89 | −153 | −55 | A | C |
| ATOM | 2839 | CH2 | TRP | A | 591 | 17.177 | 2.755 | 13.879 | 1.00 | 14.22 | | A | C |
| ANISOU | 2839 | CH2 | TRP | A | 591 | 1867 | 1669 | 1866 | 134 | −135 | −110 | A | C |
| ATOM | 2841 | CZ2 | TRP | A | 591 | 16.659 | 2.090 | 14.962 | 1.00 | 13.23 | | A | C |
| ANISOU | 2841 | CZ2 | TRP | A | 591 | 1675 | 1669 | 1681 | 211 | −83 | −98 | A | C |
| ATOM | 2843 | C | TRP | A | 591 | 14.015 | −3.349 | 10.940 | 1.00 | 9.10 | | A | C |
| ANISOU | 2843 | C | TRP | A | 591 | 1120 | 1220 | 1117 | −80 | 106 | 27 | A | C |
| ATOM | 2844 | O | TRP | A | 591 | 13.203 | −4.078 | 11.509 | 1.00 | 9.60 | | A | O |
| ANISOU | 2844 | O | TRP | A | 591 | 1272 | 1285 | 1088 | −186 | 226 | 128 | A | O |
| ATOM | 2845 | N | MET | A | 592 | 14.123 | −3.286 | 9.615 | 1.00 | 8.97 | | A | N |
| ANISOU | 2845 | N | MET | A | 592 | 1068 | 1302 | 1038 | −63 | 34 | −14 | A | N |
| ATOM | 2847 | CA | MET | A | 592 | 13.138 | −3.872 | 8.697 | 1.00 | 8.99 | | A | C |
| ANISOU | 2847 | CA | MET | A | 592 | 1065 | 1226 | 1124 | −39 | −14 | 28 | A | C |
| ATOM | 2849 | CB | MET | A | 592 | 12.955 | −2.949 | 7.475 | 1.00 | 9.42 | | A | C |
| ANISOU | 2849 | CB | MET | A | 592 | 1080 | 1294 | 1203 | −52 | 26 | 62 | A | C |
| ATOM | 2852 | CG | MET | A | 592 | 12.048 | −1.807 | 7.767 | 1.00 | 11.87 | | A | C |
| ANISOU | 2852 | CG | MET | A | 592 | 1370 | 1533 | 1605 | 31 | −2 | 55 | A | C |
| ATOM | 2855 | SD | MET | A | 592 | 12.167 | −0.465 | 6.588 | 1.00 | 13.21 | | A | S |
| ANISOU | 2855 | SD | MET | A | 592 | 1673 | 1573 | 1773 | 144 | −201 | 79 | A | S |
| ATOM | 2856 | CE | MET | A | 592 | 11.778 | −1.235 | 5.042 | 1.00 | 12.86 | | A | C |
| ANISOU | 2856 | CE | MET | A | 592 | 1482 | 1741 | 1663 | −52 | −90 | 106 | A | C |
| ATOM | 2860 | C | MET | A | 592 | 13.463 | −5.281 | 8.206 | 1.00 | 9.02 | | A | C |
| ANISOU | 2860 | C | MET | A | 592 | 1056 | 1225 | 1146 | −35 | −13 | 9 | A | C |
| ATOM | 2861 | O | MET | A | 592 | 14.625 | −5.652 | 8.029 | 1.00 | 10.06 | | A | O |
| ANISOU | 2861 | O | MET | A | 592 | 1321 | 1196 | 1304 | 119 | 13 | −5 | A | O |
| ATOM | 2862 | N | SER | A | 593 | 12.406 | −6.058 | 7.968 | 1.00 | 9.29 | | A | N |
| ANISOU | 2862 | N | SER | A | 593 | 1059 | 1295 | 1173 | −41 | −22 | 40 | A | N |
| ATOM | 2864 | CA | SER | A | 593 | 12.542 | −7.395 | 7.428 | 1.00 | 9.43 | | A | C |
| ANISOU | 2864 | CA | SER | A | 593 | 1132 | 1284 | 1164 | −23 | −2 | −1 | A | C |
| ATOM | 2866 | CB | SER | A | 593 | 11.223 | −8.158 | 7.405 | 1.00 | 10.04 | | A | C |
| ANISOU | 2866 | CB | SER | A | 593 | 1210 | 1360 | 1243 | −15 | 18 | −13 | A | C |
| ATOM | 2869 | OG | SER | A | 593 | 10.389 | −7.727 | 6.355 | 1.00 | 11.29 | | A | O |
| ANISOU | 2869 | OG | SER | A | 593 | 1284 | 1637 | 1367 | −38 | 19 | 0 | A | O |
| ATOM | 2871 | C | SER | A | 593 | 13.100 | −7.345 | 6.017 | 1.00 | 9.22 | | A | C |
| ANISOU | 2871 | C | SER | A | 593 | 1105 | 1251 | 1146 | −45 | 41 | 85 | A | C |
| ATOM | 2872 | O | SER | A | 593 | 12.950 | −6.322 | 5.324 | 1.00 | 9.98 | | A | O |
| ANISOU | 2872 | O | SER | A | 593 | 1353 | 1417 | 1019 | 10 | 35 | 73 | A | O |
| ATOM | 2873 | N | PRO | A | 594 | 13.718 | −8.436 | 5.576 | 1.00 | 8.91 | | A | N |
| ANISOU | 2873 | N | PRO | A | 594 | 1097 | 1209 | 1077 | −50 | 71 | 65 | A | N |
| ATOM | 2874 | CA | PRO | A | 594 | 14.227 | −8.463 | 4.188 | 1.00 | 9.59 | | A | C |
| ANISOU | 2874 | CA | PRO | A | 594 | 1197 | 1262 | 1185 | −47 | 55 | 19 | A | C |
| ATOM | 2876 | CB | PRO | A | 594 | 14.833 | −9.853 | 4.045 | 1.00 | 10.47 | | A | C |
| ANISOU | 2876 | CB | PRO | A | 594 | 1271 | 1343 | 1360 | −20 | 73 | 48 | A | C |
| ATOM | 2879 | CG | PRO | A | 594 | 15.100 | −10.293 | 5.442 | 1.00 | 11.63 | | A | C |
| ANISOU | 2879 | CG | PRO | A | 594 | 1509 | 1514 | 1393 | 13 | 31 | 2 | A | C |
| ATOM | 2882 | CD | PRO | A | 594 | 14.062 | −9.666 | 6.321 | 1.00 | 10.26 | | A | C |
| ANISOU | 2882 | CD | PRO | A | 594 | 1211 | 1294 | 1390 | −94 | 8 | −6 | A | C |
| ATOM | 2885 | C | PRO | A | 594 | 13.157 | −8.210 | 3.160 | 1.00 | 9.30 | | A | C |
| ANISOU | 2885 | C | PRO | A | 594 | 1180 | 1239 | 1112 | −36 | 74 | −34 | A | C |
| ATOM | 2886 | O | PRO | A | 594 | 13.400 | −7.501 | 2.210 | 1.00 | 10.37 | | A | O |
| ANISOU | 2886 | O | PRO | A | 594 | 1249 | 1378 | 1312 | 18 | −47 | 150 | A | O |
| ATOM | 2887 | N | GLU | A | 595 | 11.976 | −8.763 | 3.347 | 1.00 | 10.12 | | A | N |
| ANISOU | 2887 | N | GLU | A | 595 | 1297 | 1385 | 1163 | −32 | −11 | 27 | A | N |
| ATOM | 2889 | CA | GLU | A | 595 | 10.911 | −8.579 | 2.375 | 1.00 | 10.05 | | A | C |
| ANISOU | 2889 | CA | GLU | A | 595 | 1222 | 1302 | 1292 | −13 | −47 | O | A | C |
| ATOM | 2891 | CB | GLU | A | 595 | 9.809 | −9.610 | 2.584 | 1.00 | 10.60 | | A | C |
| ANISOU | 2891 | CB | GLU | A | 595 | 1315 | 1411 | 1298 | −52 | −7 | 42 | A | C |
| ATOM | 2894 | CG | GLU | A | 595 | 8.953 | −9.471 | 3.827 | 1.00 | 11.26 | | A | C |
| ANISOU | 2894 | CG | GLU | A | 595 | 1336 | 1498 | 1442 | −130 | 5 | −6 | A | C |
| ATOM | 2897 | CD | GLU | A | 595 | 9.485 | −10.178 | 5.054 | 1.00 | 11.56 | | A | C |
| ANISOU | 2897 | CD | GLU | A | 595 | 1394 | 1580 | 1418 | 28 | 28 | 35 | A | C |
| ATOM | 2898 | OE1 | GLU | A | 595 | 10.685 | −10.577 | 5.091 | 1.00 | 11.13 | | A | O |
| ANISOU | 2898 | OE1 | GLU | A | 595 | 1434 | 1517 | 1277 | −69 | 73 | 78 | A | O |
| ATOM | 2899 | OE2 | GLU | A | 595 | 8.670 | −10.321 | 6.019 | 1.00 | 11.62 | | A | O |
| ANISOU | 2899 | OE2 | GLU | A | 595 | 1484 | 1457 | 1472 | 42 | 121 | −5 | A | O |
| ATOM | 2900 | C | GLU | A | 595 | 10.383 | −7.144 | 2.371 | 1.00 | 9.21 | | A | C |
| ANISOU | 2900 | C | GLU | A | 595 | 1136 | 1262 | 1100 | 37 | 30 | 8 | A | C |
| ATOM | 2901 | O | GLU | A | 595 | 9.883 | −6.656 | 1.347 | 1.00 | 9.76 | | A | O |
| ANISOU | 2901 | O | GLU | A | 595 | 1097 | 1442 | 1167 | −2 | −68 | −41 | A | O |
| ATOM | 2902 | N | SER | A | 596 | 10.500 | −6.452 | 3.501 | 1.00 | 9.67 | | A | N |
| ANISOU | 2902 | N | SER | A | 596 | 1235 | 1236 | 1203 | −7 | −38 | 28 | A | N |
| ATOM | 2904 | CA | SER | A | 596 | 10.129 | −5.052 | 3.558 | 1.00 | 9.89 | | A | C |
| ANISOU | 2904 | CA | SER | A | 596 | 1196 | 1260 | 1299 | −21 | −28 | 25 | A | C |
| ATOM | 2906 | CB | SER | A | 596 | 10.008 | −4.594 | 5.008 | 1.00 | 10.23 | | A | C |
| ANISOU | 2906 | CB | SER | A | 596 | 1287 | 1329 | 1268 | −75 | 105 | 98 | A | C |
| ATOM | 2909 | OG | SER | A | 596 | 9.052 | −5.376 | 5.764 | 1.00 | 10.32 | | A | O |
| ANISOU | 2909 | OG | SER | A | 596 | 1213 | 1403 | 1302 | −145 | 245 | 197 | A | O |
| ATOM | 2911 | C | SER | A | 596 | 11.171 | −4.200 | 2.827 | 1.00 | 9.96 | | A | C |
| ANISOU | 2911 | C | SER | A | 596 | 1248 | 1278 | 1255 | −10 | −22 | 46 | A | C |
| ATOM | 2912 | O | SER | A | 596 | 10.830 | −3.244 | 2.133 | 1.00 | 9.77 | | A | O |
| ANISOU | 2912 | O | SER | A | 596 | 1240 | 1189 | 1281 | −62 | −68 | 33 | A | O |
| ATOM | 2913 | N | ILE | A | 597 | 12.441 | −4.557 | 2.970 | 1.00 | 10.01 | | A | N |
| ANISOU | 2913 | N | ILE | A | 597 | 1255 | 1266 | 1280 | −33 | −36 | 89 | A | N |
| ATOM | 2915 | CA | ILE | A | 597 | 13.514 | −3.836 | 2.286 | 1.00 | 9.56 | | A | C |
| ANISOU | 2915 | CA | ILE | A | 597 | 1210 | 1283 | 1136 | −35 | 10 | 0 | A | C |
| ATOM | 2917 | CB | ILE | A | 597 | 14.901 | −4.252 | 2.834 | 1.00 | 9.88 | | A | C |
| ANISOU | 2917 | CB | ILE | A | 597 | 1284 | 1340 | 1130 | −70 | −42 | 60 | A | C |
| ATOM | 2919 | CG1 | ILE | A | 597 | 15.037 | −3.816 | 4.304 | 1.00 | 9.44 | | A | C |
| ANISOU | 2919 | CG1 | ILE | A | 597 | 1175 | 1212 | 1197 | 2 | 76 | −30 | A | C |
| ATOM | 2922 | CD1 | ILE | A | 597 | 16.240 | −4.356 | 4.994 | 1.00 | 10.48 | | A | C |
| ANISOU | 2922 | CD1 | ILE | A | 597 | 1424 | 1480 | 1077 | 41 | −61 | −30 | A | C |
| ATOM | 2926 | CG2 | ILE | A | 597 | 16.019 | −3.627 | 1.970 | 1.00 | 10.27 | | A | C |
| ANISOU | 2926 | CG2 | ILE | A | 597 | 1248 | 1426 | 1228 | −93 | −42 | 58 | A | C |
| ATOM | 2930 | C | ILE | A | 597 | 13.438 | −4.070 | 0.778 | 1.00 | 9.98 | | A | C |
| ANISOU | 2930 | C | ILE | A | 597 | 1320 | 1345 | 1126 | −41 | 7 | 69 | A | C |
| ATOM | 2931 | O | ILE | A | 597 | 13.494 | −3.129 | −0.030 | 1.00 | 10.68 | | A | O |
| ANISOU | 2931 | O | ILE | A | 597 | 1422 | 1278 | 1357 | −44 | −20 | 162 | A | O |
| ATOM | 2932 | N | ASN | A | 598 | 13.296 | −5.320 | 0.405 | 1.00 | 10.21 | | A | N |
| ANISOU | 2932 | N | ASN | A | 598 | 1380 | 1360 | 1138 | −50 | 35 | 30 | A | N |
| ATOM | 2934 | CA | ASN | A | 598 | 13.343 | −5.707 | −1.024 | 1.00 | 10.75 | | A | C |
| ANISOU | 2934 | CA | ASN | A | 598 | 1425 | 1458 | 1199 | −28 | 8 | 11 | A | C |
| ATOM | 2936 | CB | ASN | A | 598 | 13.584 | −7.212 | −1.153 | 1.00 | 10.68 | | A | C |
| ANISOU | 2936 | CB | ASN | A | 598 | 1402 | 1431 | 1225 | −25 | 49 | −13 | A | C |
| ATOM | 2939 | CG | ASN | A | 598 | 15.020 | −7.605 | −0.903 | 1.00 | 12.94 | | A | C |
| ANISOU | 2939 | CG | ASN | A | 598 | 1649 | 1748 | 1519 | −65 | −177 | 39 | A | C |
| ATOM | 2940 | OD1 | ASN | A | 598 | 15.945 | −6.851 | −1.204 | 1.00 | 14.53 | | A | O |
| ANISOU | 2940 | OD1 | ASN | A | 598 | 1544 | 2190 | 1787 | −17 | 89 | −14 | A | O |
| ATOM | 2941 | ND2 | ASN | A | 598 | 15.217 | −8.832 | −0.397 | 1.00 | 14.73 | | A | N |
| ANISOU | 2941 | ND2 | ASN | A | 598 | 2011 | 1799 | 1785 | −55 | −246 | 29 | A | N |
| ATOM | 2944 | C | ASN | A | 598 | 12.079 | −5.365 | −1.807 | 1.00 | 11.61 | | A | C |
| ANISOU | 2944 | C | ASN | A | 598 | 1470 | 1606 | 1334 | −17 | 23 | 58 | A | C |
| ATOM | 2945 | O | ASN | A | 598 | 12.150 | −4.927 | −2.954 | 1.00 | 11.95 | | A | O |
| ANISOU | 2945 | O | ASN | A | 598 | 1598 | 1738 | 1203 | −74 | 48 | 74 | A | O |
| ATOM | 2946 | N | PHE | A | 599 | 10.915 | −5.571 | −1.192 | 1.00 | 11.13 | | A | N |
| ANISOU | 2946 | N | PHE | A | 599 | 1371 | 1614 | 1244 | −82 | −19 | 43 | A | N |
| ATOM | 2948 | CA | PHE | A | 599 | 9.643 | −5.530 | −1.925 | 1.00 | 11.86 | | A | C |
| ANISOU | 2948 | CA | PHE | A | 599 | 1459 | 1606 | 1441 | −1 | −54 | 39 | A | C |
| ATOM | 2950 | CB | PHE | A | 599 | 9.109 | −6.949 | −2.112 | 1.00 | 12.27 | | A | C |
| ANISOU | 2950 | CB | PHE | A | 599 | 1511 | 1667 | 1480 | −4 | −80 | −27 | A | C |
| ATOM | 2953 | CG | PHE | A | 599 | 10.116 | −7.904 | −2.688 | 1.00 | 13.95 | | A | C |
| ANISOU | 2953 | CG | PHE | A | 599 | 1691 | 1826 | 1781 | 0 | −47 | −36 | A | C |
| ATOM | 2954 | CD1 | PHE | A | 599 | 10.758 | −7.627 | −3.890 | 1.00 | 14.56 | | A | C |
| ANISOU | 2954 | CD1 | PHE | A | 599 | 1774 | 1899 | 1856 | 34 | 7 | −17 | A | C |
| ATOM | 2956 | CE1 | PHE | A | 599 | 11.700 | −8.503 | −4.401 | 1.00 | 16.51 | | A | C |
| ANISOU | 2956 | CE1 | PHE | A | 599 | 2048 | 2000 | 2223 | 24 | 41 | −25 | A | C |
| ATOM | 2958 | CZ | PHE | A | 599 | 11.999 | −9.653 | −3.717 | 1.00 | 15.93 | | A | C |
| ANISOU | 2958 | CZ | PHE | A | 599 | 1918 | 2030 | 2105 | 101 | 41 | −31 | A | C |
| ATOM | 2960 | CE2 | PHE | A | 599 | 11.372 | −9.948 | −2.549 | 1.00 | 16.28 | | A | C |
| ANISOU | 2960 | CE2 | PHE | A | 599 | 1991 | 2025 | 2166 | 1 | −15 | −19 | A | C |
| ATOM | 2962 | CD2 | PHE | A | 599 | 10.447 | −9.066 | −2.013 | 1.00 | 14.63 | | A | C |
| ANISOU | 2962 | CD2 | PHE | A | 599 | 1831 | 1797 | 1928 | −41 | 5 | −23 | A | C |
| ATOM | 2964 | C | PHE | A | 599 | 8.584 | −4.682 | −1.247 | 1.00 | 11.77 | | A | C |
| ANISOU | 2964 | C | PHE | A | 599 | 1463 | 1608 | 1400 | −24 | −39 | 33 | A | C |
| ATOM | 2965 | O | PHE | A | 599 | 7.439 | −4.693 | −1.660 | 1.00 | 12.18 | | A | O |
| ANISOU | 2965 | O | PHE | A | 599 | 1468 | 1661 | 1496 | −153 | −124 | 52 | A | O |
| ATOM | 2966 | N | ARG | A | 600 | 8.969 | −3.947 | −0.204 | 1.00 | 11.50 | | A | N |
| ANISOU | 2966 | N | ARG | A | 600 | 1459 | 1527 | 1382 | 18 | −42 | −6 | A | N |
| ATOM | 2968 | CA | ARG | A | 600 | 8.033 | −3.173 | 0.604 | 1.00 | 11.53 | | A | C |
| ANISOU | 2968 | CA | ARG | A | 600 | 1463 | 1514 | 1401 | 22 | −24 | 24 | A | C |
| ATOM | 2970 | CB | ARG | A | 600 | 7.636 | −1.887 | −0.143 | 1.00 | 11.85 | | A | C |
| ANISOU | 2970 | CB | ARG | A | 600 | 1556 | 1479 | 1467 | −23 | 37 | 48 | A | C |
| ATOM | 2973 | CG | ARG | A | 600 | 8.808 | −0.969 | −0.430 | 1.00 | 11.41 | | A | C |
| ANISOU | 2973 | CG | ARG | A | 600 | 1458 | 1483 | 1393 | 42 | −17 | 84 | A | C |
| ATOM | 2976 | CD | ARG | A | 600 | 9.327 | −0.272 | 0.803 | 1.00 | 13.18 | | A | C |
| ANISOU | 2976 | CD | ARG | A | 600 | 1633 | 1644 | 1730 | 22 | −38 | −12 | A | C |
| ATOM | 2979 | NE | ARG | A | 600 | 10.395 | 0.643 | 0.428 | 1.00 | 14.17 | | A | N |
| ANISOU | 2979 | NE | ARG | A | 600 | 1837 | 1614 | 1929 | 10 | −3 | 133 | A | N |
| ATOM | 2981 | CZ | ARG | A | 600 | 11.695 | 0.379 | 0.489 | 1.00 | 15.98 | | A | C |
| ANISOU | 2981 | CZ | ARG | A | 600 | 1894 | 1988 | 2189 | 25 | −3 | 14 | A | C |
| ATOM | 2982 | NH1 | ARG | A | 600 | 12.141 | −0.779 | 0.959 | 1.00 | 15.36 | | A | N |
| ANISOU | 2982 | NH1 | ARG | A | 600 | 1832 | 1945 | 2057 | −104 | −33 | 126 | A | N |
| ATOM | 2985 | NH2 | ARG | A | 600 | 12.561 | 1.294 | 0.074 | 1.00 | 17.80 | | A | N |
| ANISOU | 2985 | NH2 | ARG | A | 600 | 2293 | 2177 | 2291 | −95 | 1 | 95 | A | N |
| ATOM | 2988 | C | ARG | A | 600 | 6.813 | −3.997 | 0.991 | 1.00 | 12.03 | | A | C |
| ANISOU | 2988 | C | ARG | A | 600 | 1525 | 1555 | 1488 | 4 | 21 | 66 | A | C |
| ATOM | 2989 | O | ARG | A | 600 | 5.667 | −3.540 | 0.899 | 1.00 | 12.71 | | A | O |
| ANISOU | 2989 | O | ARG | A | 600 | 1640 | 1649 | 1538 | 89 | 68 | 216 | A | O |
| ATOM | 2990 | N | ARG | A | 601 | 7.089 | −5.218 | 1.452 | 1.00 | 11.58 | | A | N |
| ANISOU | 2990 | N | ARG | A | 601 | 1417 | 1563 | 1419 | 7 | −49 | 58 | A | N |
| ATOM | 2992 | CA | ARG | A | 601 | 6.078 | −6.128 | 1.965 | 1.00 | 11.44 | | A | C |
| ANISOU | 2992 | CA | ARG | A | 601 | 1424 | 1551 | 1369 | −12 | −37 | 53 | A | C |
| ATOM | 2994 | CB | ARG | A | 601 | 6.463 | −7.563 | 1.637 | 1.00 | 12.67 | | A | C |
| ANISOU | 2994 | CB | ARG | A | 601 | 1588 | 1663 | 1561 | −10 | −91 | −57 | A | C |
| ATOM | 2997 | CG | ARG | A | 601 | 5.472 | −8.612 | 2.069 | 1.00 | 14.91 | | A | C |
| ANISOU | 2997 | CG | ARG | A | 601 | 1851 | 1995 | 1818 | −34 | −14 | 61 | A | C |
| ATOM | 3000 | CD | ARG | A | 601 | 5.697 | −9.952 | 1.382 | 1.00 | 17.74 | | A | C |
| ANISOU | 3000 | CD | ARG | A | 601 | 2258 | 2106 | 2374 | 4 | 7 | −3 | A | C |
| ATOM | 3003 | NE | ARG | A | 601 | 5.514 | −9.784 | −0.057 | 1.00 | 19.66 | | A | N |
| ANISOU | 3003 | NE | ARG | A | 601 | 2617 | 2382 | 2468 | 87 | −41 | −10 | A | N |
| ATOM | 3005 | CZ | ARG | A | 601 | 6.285 | −10.267 | −1.029 | 1.00 | 21.00 | | A | C |
| ANISOU | 3005 | CZ | ARG | A | 601 | 2561 | 2753 | 2663 | −11 | 86 | 53 | A | C |
| ATOM | 3006 | NH1 | ARG | A | 601 | 7.357 | −11.018 | −0.784 | 1.00 | 22.47 | | A | N |
| ANISOU | 3006 | NH1 | ARG | A | 601 | 2809 | 2782 | 2946 | 79 | −1 | 100 | A | N |
| ATOM | 3009 | NH2 | ARG | A | 601 | 5.983 | −9.979 | −2.280 | 1.00 | 19.82 | | A | N |
| ANISOU | 3009 | NH2 | ARG | A | 601 | 2298 | 2580 | 2652 | 95 | −57 | 78 | A | N |
| ATOM | 3012 | C | ARG | A | 601 | 6.063 | −5.963 | 3.470 | 1.00 | 11.15 | | A | C |
| ANISOU | 3012 | C | ARG | A | 601 | 1403 | 1500 | 1332 | −36 | −18 | 53 | A | C |
| ATOM | 3013 | O | ARG | A | 601 | 7.066 | −6.212 | 4.126 | 1.00 | 11.96 | | A | O |
| ANISOU | 3013 | O | ARG | A | 601 | 1567 | 1654 | 1321 | 43 | 73 | 60 | A | O |
| ATOM | 3014 | N | PHE | A | 602 | 4.931 | −5.518 | 3.991 | 1.00 | 10.72 | | A | N |
| ANISOU | 3014 | N | PHE | A | 602 | 1378 | 1494 | 1199 | 36 | 21 | 77 | A | N |
| ATOM | 3016 | CA | PHE | A | 602 | 4.763 | −5.308 | 5.412 | 1.00 | 10.99 | | A | C |
| ANISOU | 3016 | CA | PHE | A | 602 | 1305 | 1548 | 1321 | −15 | 23 | 61 | A | C |
| ATOM | 3018 | CB | PHE | A | 602 | 4.545 | −3.851 | 5.713 | 1.00 | 11.03 | | A | C |
| ANISOU | 3018 | CB | PHE | A | 602 | 1315 | 1509 | 1364 | −7 | 22 | 36 | A | C |
| ATOM | 3021 | CG | PHE | A | 602 | 5.689 | −2.951 | 5.331 | 1.00 | 11.43 | | A | C |
| ANISOU | 3021 | CG | PHE | A | 602 | 1386 | 1493 | 1461 | 0 | 52 | 74 | A | C |
| ATOM | 3022 | CD1 | PHE | A | 602 | 6.668 | −2.610 | 6.273 | 1.00 | 13.11 | | A | C |
| ANISOU | 3022 | CD1 | PHE | A | 602 | 1529 | 1757 | 1691 | −1 | −61 | 185 | A | C |
| ATOM | 3024 | CE1 | PHE | A | 602 | 7.746 | −1.771 | 5.922 | 1.00 | 13.50 | | A | C |
| ANISOU | 3024 | CE1 | PHE | A | 602 | 1500 | 1892 | 1736 | 21 | 45 | −12 | A | C |
| ATOM | 3026 | CZ | PHE | A | 602 | 7.788 | −1.239 | 4.645 | 1.00 | 14.36 | | A | C |
| ANISOU | 3026 | CZ | PHE | A | 602 | 1731 | 1858 | 1865 | 51 | 24 | 94 | A | C |
| ATOM | 3028 | CE2 | PHE | A | 602 | 6.813 | −1.558 | 3.712 | 1.00 | 13.34 | | A | C |
| ANISOU | 3028 | CE2 | PHE | A | 602 | 1681 | 1625 | 1762 | 52 | 114 | −48 | A | C |
| ATOM | 3030 | CD2 | PHE | A | 602 | 5.775 | −2.418 | 4.052 | 1.00 | 14.19 | | A | C |
| ANISOU | 3030 | CD2 | PHE | A | 602 | 1724 | 1964 | 1702 | −10 | 36 | 88 | A | C |
| ATOM | 3032 | C | PHE | A | 602 | 3.551 | −6.087 | 5.905 | 1.00 | 10.54 | | A | C |
| ANISOU | 3032 | C | PHE | A | 602 | 1148 | 1469 | 1386 | −13 | −1 | 116 | A | C |
| ATOM | 3033 | O | PHE | A | 602 | 2.407 | −5.842 | 5.488 | 1.00 | 12.65 | | A | O |
| ANISOU | 3033 | O | PHE | A | 602 | 1365 | 1889 | 1551 | 108 | −29 | 211 | A | O |
| ATOM | 3034 | N | THR | A | 603 | 3.811 | −7.023 | 6.796 | 1.00 | 11.08 | | A | N |
| ANISOU | 3034 | N | THR | A | 603 | 1222 | 1582 | 1405 | −24 | 4 | 106 | A | N |
| ATOM | 3036 | CA | THR | A | 603 | 2.816 | −7.938 | 7.319 | 1.00 | 11.28 | | A | C |
| ANISOU | 3036 | CA | THR | A | 603 | 1328 | 1523 | 1433 | −28 | −70 | 66 | A | C |
| ATOM | 3038 | CB | THR | A | 603 | 2.983 | −9.312 | 6.670 | 1.00 | 12.02 | | A | C |
| ANISOU | 3038 | CB | THR | A | 603 | 1395 | 1663 | 1507 | −1 | 31 | −12 | A | C |
| ATOM | 3040 | OG1 | THR | A | 603 | 4.240 | −9.900 | 7.083 | 1.00 | 13.58 | | A | O |
| ANISOU | 3040 | OG1 | THR | A | 603 | 1799 | 1882 | 1478 | 121 | −99 | 64 | A | O |
| ATOM | 3042 | CG2 | THR | A | 603 | 2.992 | −9.233 | 5.109 | 1.00 | 13.80 | | A | C |
| ANISOU | 3042 | CG2 | THR | A | 603 | 1655 | 1915 | 1669 | −17 | −57 | 7 | A | C |
| ATOM | 3046 | C | THR | A | 603 | 3.050 | −8.096 | 8.812 | 1.00 | 10.70 | | A | C |
| ANISOU | 3046 | C | THR | A | 603 | 1306 | 1392 | 1365 | −8 | −32 | 68 | A | C |
| ATOM | 3047 | O | THR | A | 603 | 3.999 | −7.564 | 9.368 | 1.00 | 10.54 | | A | O |
| ANISOU | 3047 | O | THR | A | 603 | 1282 | 1352 | 1369 | −5 | 25 | −23 | A | O |
| ATOM | 3048 | N | THR | A | 604 | 2.189 | −8.848 | 9.478 | 1.00 | 10.68 | | A | N |
| ANISOU | 3048 | N | THR | A | 604 | 1218 | 1508 | 1329 | −17 | 23 | 81 | A | N |
| ATOM | 3050 | CA | THR | A | 604 | 2.465 | −9.157 | 10.874 | 1.00 | 10.95 | | A | C |
| ANISOU | 3050 | CA | THR | A | 604 | 1327 | 1474 | 1358 | −15 | 13 | 41 | A | C |
| ATOM | 3052 | CB | THR | A | 604 | 1.306 | −9.959 | 11.474 | 1.00 | 12.36 | | A | C |
| ANISOU | 3052 | CB | THR | A | 604 | 1400 | 1764 | 1533 | 21 | 54 | 140 | A | C |
| ATOM | 3054 | OG1 | THR | A | 604 | 0.140 | −9.113 | 11.538 | 1.00 | 15.22 | | A | O |
| ANISOU | 3054 | OG1 | THR | A | 604 | 1720 | 2173 | 1887 | 218 | 80 | −10 | A | O |
| ATOM | 3056 | CG2 | THR | A | 604 | 1.597 | −10.335 | 12.915 | 1.00 | 14.21 | | A | C |
| ANISOU | 3056 | CG2 | THR | A | 604 | 1763 | 1889 | 1746 | −3 | 32 | 55 | A | C |
| ATOM | 3060 | C | THR | A | 604 | 3.814 | −9.877 | 11.035 | 1.00 | 10.39 | | A | C |
| ANISOU | 3060 | C | THR | A | 604 | 1301 | 1384 | 1260 | −57 | 34 | 32 | A | C |
| ATOM | 3061 | O | THR | A | 604 | 4.513 | −9.634 | 12.009 | 1.00 | 10.09 | | A | O |
| ANISOU | 3061 | O | THR | A | 604 | 1291 | 1435 | 1106 | −33 | 61 | −16 | A | O |
| ATOM | 3062 | N | ALA | A | 605 | 4.208 | −10.710 | 10.062 | 1.00 | 9.88 | | A | N |
| ANISOU | 3062 | N | ALA | A | 605 | 1244 | 1284 | 1226 | −46 | −3 | 22 | A | N |
| ATOM | 3064 | CA | ALA | A | 605 | 5.500 | −11.361 | 10.095 | 1.00 | 9.56 | | A | C |
| ANISOU | 3064 | CA | ALA | A | 605 | 1219 | 1168 | 1244 | −45 | 46 | 14 | A | C |
| ATOM | 3066 | CB | ALA | A | 605 | 5.625 | −12.414 | 9.019 | 1.00 | 9.71 | | A | C |
| ANISOU | 3066 | CB | ALA | A | 605 | 1094 | 1242 | 1353 | −73 | 5 | −81 | A | C |
| ATOM | 3070 | C | ALA | A | 605 | 6.675 | −10.385 | 9.998 | 1.00 | 9.33 | | A | C |
| ANISOU | 3070 | C | ALA | A | 605 | 1124 | 1231 | 1190 | −37 | −11 | −7 | A | C |
| ATOM | 3071 | O | ALA | A | 605 | 7.711 | −10.608 | 10.637 | 1.00 | 8.88 | | A | O |
| ANISOU | 3071 | O | ALA | A | 605 | 1140 | 1174 | 1059 | 41 | −46 | 60 | A | O |
| ATOM | 3072 | N | SER | A | 606 | 6.512 | −9.307 | 9.241 | 1.00 | 8.76 | | A | N |
| ANISOU | 3072 | N | SER | A | 606 | 1154 | 1124 | 1049 | −29 | 11 | −27 | A | N |
| ATOM | 3074 | CA | SER | A | 606 | 7.539 | −8.278 | 9.219 | 1.00 | 8.84 | | A | C |
| ANISOU | 3074 | CA | SER | A | 606 | 1100 | 1210 | 1046 | −25 | −9 | 32 | A | C |
| ATOM | 3076 | CB | SER | A | 606 | 7.487 | −7.363 | 7.991 | 1.00 | 9.46 | | A | C |
| ANISOU | 3076 | CB | SER | A | 606 | 1308 | 1122 | 1160 | −61 | 73 | 61 | A | C |
| ATOM | 3079 | OG | SER | A | 606 | 6.365 | −6.519 | 7.970 | 1.00 | 12.44 | | A | O |
| ANISOU | 3079 | OG | SER | A | 606 | 1548 | 1453 | 1726 | 70 | 55 | 153 | A | O |
| ATOM | 3081 | C | SER | A | 606 | 7.590 | −7.503 | 10.539 | 1.00 | 8.37 | | A | C |
| ANISOU | 3081 | C | SER | A | 606 | 1078 | 1081 | 1020 | −8 | −5 | 24 | A | C |
| ATOM | 3082 | O | SER | A | 606 | 8.670 | −7.107 | 10.981 | 1.00 | 9.31 | | A | O |
| ANISOU | 3082 | O | SER | A | 606 | 1114 | 1306 | 1116 | −30 | −37 | 167 | A | O |
| ATOM | 3083 | N | ASP | A | 607 | 6.433 | −7.317 | 11.174 | 1.00 | 8.77 | | A | N |
| ANISOU | 3083 | N | ASP | A | 607 | 1027 | 1217 | 1086 | −30 | 8 | 47 | A | N |
| ATOM | 3085 | CA | ASP | A | 607 | 6.384 | −6.745 | 12.523 | 1.00 | 8.49 | | A | C |
| ANISOU | 3085 | CA | ASP | A | 607 | 1053 | 1109 | 1061 | −60 | 50 | 41 | A | C |
| ATOM | 3087 | CB | ASP | A | 607 | 4.954 | −6.516 | 13.002 | 1.00 | 8.95 | | A | C |
| ANISOU | 3087 | CB | ASP | A | 607 | 1100 | 1190 | 1108 | 30 | −32 | −3 | A | C |
| ATOM | 3090 | CG | ASP | A | 607 | 4.360 | −5.209 | 12.593 | 1.00 | 10.83 | | A | C |
| ANISOU | 3090 | CG | ASP | A | 607 | 1321 | 1330 | 1461 | 26 | 120 | 7 | A | C |
| ATOM | 3091 | OD1 | ASP | A | 607 | 5.011 | −4.305 | 11.994 | 1.00 | 13.04 | | A | O |
| ANISOU | 3091 | OD1 | ASP | A | 607 | 1672 | 1415 | 1865 | 89 | −72 | 76 | A | O |
| ATOM | 3092 | OD2 | ASP | A | 607 | 3.139 | −5.054 | 12.856 | 1.00 | 13.25 | | A | O |
| ANISOU | 3092 | OD2 | ASP | A | 607 | 1351 | 1773 | 1908 | 156 | 134 | 269 | A | O |
| ATOM | 3093 | C | ASP | A | 607 | 7.111 | −7.650 | 13.546 | 1.00 | 8.81 | | A | C |
| ANISOU | 3093 | C | ASP | A | 607 | 1106 | 1106 | 1136 | −29 | 4 | 21 | A | C |
| ATOM | 3094 | O | ASP | A | 607 | 7.754 | −7.143 | 14.458 | 1.00 | 8.69 | | A | O |
| ANISOU | 3094 | O | ASP | A | 607 | 1188 | 931 | 1183 | 33 | −77 | 88 | A | O |
| ATOM | 3095 | N | VAL | A | 608 | 7.008 | −8.969 | 13.395 | 1.00 | 8.75 | | A | N |
| ANISOU | 3095 | N | VAL | A | 608 | 1030 | 1131 | 1163 | 11 | −21 | 72 | A | N |
| ATOM | 3097 | CA | VAL | A | 608 | 7.718 | −9.894 | 14.282 | 1.00 | 8.19 | | A | C |
| ANISOU | 3097 | CA | VAL | A | 608 | 873 | 1051 | 1188 | 76 | 60 | 39 | A | C |
| ATOM | 3099 | CB | VAL | A | 608 | 7.320 | −11.353 | 14.019 | 1.00 | 8.71 | | A | C |
| ANISOU | 3099 | CB | VAL | A | 608 | 907 | 1144 | 1256 | −42 | 39 | 19 | A | C |
| ATOM | 3101 | CG1 | VAL | A | 608 | 8.255 | −12.340 | 14.695 | 1.00 | 8.72 | | A | C |
| ANISOU | 3101 | CG1 | VAL | A | 608 | 822 | 1138 | 1350 | −60 | 25 | 5 | A | C |
| ATOM | 3105 | CG2 | VAL | A | 608 | 5.884 | −11.612 | 14.519 | 1.00 | 9.27 | | A | C |
| ANISOU | 3105 | CG2 | VAL | A | 608 | 1017 | 1114 | 1389 | −43 | 45 | 2 | A | C |
| ATOM | 3109 | C | VAL | A | 608 | 9.237 | −9.701 | 14.158 | 1.00 | 8.48 | | A | C |
| ANISOU | 3109 | C | VAL | A | 608 | 994 | 1166 | 1060 | −4 | 45 | 33 | A | C |
| ATOM | 3110 | O | VAL | A | 608 | 9.932 | −9.625 | 15.166 | 1.00 | 8.08 | | A | O |
| ANISOU | 3110 | O | VAL | A | 608 | 889 | 1062 | 1118 | 70 | 11 | 48 | A | O |
| ATOM | 3111 | N | TRP | A | 609 | 9.729 | −9.606 | 12.914 | 1.00 | 8.33 | | A | N |
| ANISOU | 3111 | N | TRP | A | 609 | 1007 | 1249 | 908 | −41 | 26 | 37 | A | N |
| ATOM | 3113 | CA | TRP | A | 609 | 11.142 | −9.320 | 12.681 | 1.00 | 8.34 | | A | C |
| ANISOU | 3113 | CA | TRP | A | 609 | 1059 | 1133 | 977 | 22 | 17 | 21 | A | C |
| ATOM | 3115 | CB | TRP | A | 609 | 11.385 | −9.180 | 11.178 | 1.00 | 7.90 | | A | C |
| ANISOU | 3115 | CB | TRP | A | 609 | 1066 | 1075 | 858 | −48 | 94 | −113 | A | C |
| ATOM | 3118 | CG | TRP | A | 609 | 12.771 | −8.768 | 10.842 | 1.00 | 7.50 | | A | C |
| ANISOU | 3118 | CG | TRP | A | 609 | 971 | 909 | 969 | −33 | 17 | −5 | A | C |
| ATOM | 3119 | CD1 | TRP | A | 609 | 13.368 | −7.539 | 11.112 | 1.00 | 8.81 | | A | C |
| ANISOU | 3119 | CD1 | TRP | A | 609 | 1188 | 1085 | 1072 | 38 | 1 | −215 | A | C |
| ATOM | 3121 | NE1 | TRP | A | 609 | 14.670 | −7.550 | 10.672 | 1.00 | 9.39 | | A | N |
| ANISOU | 3121 | NE1 | TRP | A | 609 | 1136 | 1203 | 1229 | −39 | 41 | −112 | A | N |
| ATOM | 3123 | CE2 | TRP | A | 609 | 14.948 | −8.779 | 10.124 | 1.00 | 8.49 | | A | C |
| ANISOU | 3123 | CE2 | TRP | A | 609 | 1078 | 1034 | 1111 | −54 | −43 | −58 | A | C |
| ATOM | 3124 | CD2 | TRP | A | 609 | 13.778 | −9.573 | 10.215 | 1.00 | 8.13 | | A | C |
| ANISOU | 3124 | CD2 | TRP | A | 609 | 1005 | 1075 | 1008 | −51 | 39 | 41 | A | C |
| ATOM | 3125 | CE3 | TRP | A | 609 | 13.805 | −10.880 | 9.692 | 1.00 | 9.35 | | A | C |
| ANISOU | 3125 | CE3 | TRP | A | 609 | 1122 | 1339 | 1091 | −34 | −66 | −2 | A | C |
| ATOM | 3127 | CZ3 | TRP | A | 609 | 14.975 | −11.352 | 9.124 | 1.00 | 9.86 | | A | C |
| ANISOU | 3127 | CZ3 | TRP | A | 609 | 1363 | 1168 | 1214 | 18 | 39 | −41 | A | C |
| ATOM | 3129 | CH2 | TRP | A | 609 | 16.119 | −10.542 | 9.073 | 1.00 | 10.57 | | A | C |
| ANISOU | 3129 | CH2 | TRP | A | 609 | 1332 | 1373 | 1311 | −13 | 31 | 38 | A | C |
| ATOM | 3131 | CZ2 | TRP | A | 609 | 16.118 | −9.251 | 9.555 | 1.00 | 9.41 | | A | C |
| ANISOU | 3131 | CZ2 | TRP | A | 609 | 1017 | 1411 | 1144 | 7 | −67 | 18 | A | C |
| ATOM | 3133 | C | TRP | A | 609 | 11.524 | −8.025 | 13.419 | 1.00 | 8.26 | | A | C |
| ANISOU | 3133 | C | TRP | A | 609 | 1006 | 1063 | 1067 | −28 | 48 | 58 | A | C |
| ATOM | 3134 | O | TRP | A | 609 | 12.522 | −7.955 | 14.143 | 1.00 | 8.86 | | A | O |
| ANISOU | 3134 | O | TRP | A | 609 | 1113 | 1169 | 1082 | −1 | −5 | 40 | A | O |
| ATOM | 3135 | N | MET | A | 610 | 10.728 | −6.980 | 13.217 | 1.00 | 8.69 | | A | N |
| ANISOU | 3135 | N | MET | A | 610 | 1029 | 1163 | 1106 | 0 | 56 | 35 | A | N |
| ATOM | 3137 | CA | MET | A | 610 | 11.045 | −5.673 | 13.757 | 1.00 | 8.50 | | A | C |
| ANISOU | 3137 | CA | MET | A | 610 | 1051 | 1120 | 1058 | 1 | 22 | 39 | A | C |
| ATOM | 3139 | CB | MET | A | 610 | 10.134 | −4.623 | 13.150 | 1.00 | 9.27 | | A | C |
| ANISOU | 3139 | CB | MET | A | 610 | 1139 | 1280 | 1101 | 60 | 34 | −12 | A | C |
| ATOM | 3142 | CG | MET | A | 610 | 10.465 | −3.184 | 13.554 | 1.00 | 10.66 | | A | C |
| ANISOU | 3142 | CG | MET | A | 610 | 1372 | 1304 | 1374 | 75 | 19 | 122 | A | C |
| ATOM | 3145 | SD | MET | A | 610 | 9.370 | −1.993 | 12.813 | 1.00 | 11.18 | | A | S |
| ANISOU | 3145 | SD | MET | A | 610 | 1461 | 1256 | 1529 | 218 | 165 | 197 | A | S |
| ATOM | 3146 | CE | MET | A | 610 | 10.494 | −0.555 | 12.750 | 1.00 | 13.95 | | A | C |
| ANISOU | 3146 | CE | MET | A | 610 | 1693 | 1720 | 1888 | 12 | 180 | 244 | A | C |
| ATOM | 3150 | C | MET | A | 610 | 10.990 | −5.686 | 15.277 | 1.00 | 7.98 | | A | C |
| ANISOU | 3150 | C | MET | A | 610 | 964 | 1022 | 1045 | 27 | 8 | 93 | A | C |
| ATOM | 3151 | O | MET | A | 610 | 11.813 | −5.085 | 15.942 | 1.00 | 8.54 | | A | O |
| ANISOU | 3151 | O | MET | A | 610 | 1078 | 1087 | 1077 | −42 | −4 | 80 | A | O |
| ATOM | 3152 | N | PHE | A | 611 | 10.020 | −6.403 | 15.820 | 1.00 | 7.88 | | A | N |
| ANISOU | 3152 | N | PHE | A | 611 | 971 | 966 | 1056 | −6 | 14 | 51 | A | N |
| ATOM | 3154 | CA | PHE | A | 611 | 9.902 | −6.526 | 17.253 | 1.00 | 7.74 | | A | C |
| ANISOU | 3154 | CA | PHE | A | 611 | 954 | 964 | 1021 | 33 | 53 | 60 | A | C |
| ATOM | 3156 | CB | PHE | A | 611 | 8.656 | −7.320 | 17.632 | 1.00 | 8.72 | | A | C |
| ANISOU | 3156 | CB | PHE | A | 611 | 1082 | 1057 | 1173 | 50 | 108 | 24 | A | C |
| ATOM | 3159 | CG | PHE | A | 611 | 8.637 | −7.708 | 19.069 | 1.00 | 8.66 | | A | C |
| ANISOU | 3159 | CG | PHE | A | 611 | 1036 | 1145 | 1107 | 62 | −101 | −66 | A | C |
| ATOM | 3160 | CD1 | PHE | A | 611 | 8.388 | −6.763 | 20.065 | 1.00 | 9.74 | | A | C |
| ANISOU | 3160 | CD1 | PHE | A | 611 | 1260 | 1295 | 1144 | 25 | 69 | −16 | A | C |
| ATOM | 3162 | CE1 | PHE | A | 611 | 8.405 | −7.126 | 21.393 | 1.00 | 9.58 | | A | C |
| ANISOU | 3162 | CE1 | PHE | A | 611 | 1340 | 1276 | 1022 | 171 | 112 | −179 | A | C |
| ATOM | 3164 | CZ | PHE | A | 611 | 8.663 | −8.428 | 21.753 | 1.00 | 9.63 | | A | C |
| ANISOU | 3164 | CZ | PHE | A | 611 | 1105 | 1320 | 1231 | −6 | 47 | 118 | A | C |
| ATOM | 3166 | CE2 | PHE | A | 611 | 8.879 | −9.378 | 20.784 | 1.00 | 10.44 | | A | C |
| ANISOU | 3166 | CE2 | PHE | A | 611 | 1387 | 1241 | 1339 | 13 | −95 | 120 | A | C |
| ATOM | 3168 | CD2 | PHE | A | 611 | 8.872 | −9.022 | 19.432 | 1.00 | 9.85 | | A | C |
| ANISOU | 3168 | CD2 | PHE | A | 611 | 1285 | 1223 | 1233 | 107 | 12 | 3 | A | C |
| ATOM | 3170 | C | PHE | A | 611 | 11.153 | −7.145 | 17.870 | 1.00 | 7.02 | | A | C |
| ANISOU | 3170 | C | PHE | A | 611 | 904 | 921 | 840 | 31 | 110 | 22 | A | C |
| ATOM | 3171 | O | PHE | A | 611 | 11.620 | −6.710 | 18.933 | 1.00 | 8.57 | | A | O |
| ANISOU | 3171 | O | PHE | A | 611 | 996 | 1108 | 1152 | 113 | 3 | 51 | A | O |
| ATOM | 3172 | N | ALA | A | 612 | 11.722 | −8.144 | 17.199 | 1.00 | 7.13 | | A | N |
| ANISOU | 3172 | N | ALA | A | 612 | 1009 | 847 | 851 | 65 | 20 | 6 | A | N |
| ATOM | 3174 | CA | ALA | A | 612 | 12.970 | −8.721 | 17.694 | 1.00 | 7.74 | | A | C |
| ANISOU | 3174 | CA | ALA | A | 612 | 990 | 1002 | 947 | 24 | 11 | 28 | A | C |
| ATOM | 3176 | CB | ALA | A | 612 | 13.295 | −10.000 | 17.023 | 1.00 | 8.53 | | A | C |
| ANISOU | 3176 | CB | ALA | A | 612 | 1135 | 993 | 1113 | 22 | 51 | 103 | A | C |
| ATOM | 3180 | C | ALA | A | 612 | 14.125 | −7.733 | 17.628 | 1.00 | 7.90 | | A | C |
| ANISOU | 3180 | C | ALA | A | 612 | 1004 | 940 | 1056 | 95 | 26 | −10 | A | C |
| ATOM | 3181 | O | ALA | A | 612 | 15.001 | −7.758 | 18.493 | 1.00 | 8.27 | | A | O |
| ANISOU | 3181 | O | ALA | A | 612 | 1085 | 1012 | 1044 | −55 | 21 | 68 | A | O |
| ATOM | 3182 | N | VAL | A | 613 | 14.148 | −6.862 | 16.615 | 1.00 | 7.47 | | A | N |
| ANISOU | 3182 | N | VAL | A | 613 | 887 | 1023 | 925 | 54 | 34 | 1 | A | N |
| ATOM | 3184 | CA | VAL | A | 613 | 15.130 | −5.771 | 16.598 | 1.00 | 7.74 | | A | C |
| ANISOU | 3184 | CA | VAL | A | 613 | 916 | 1032 | 990 | 29 | 25 | −11 | A | C |
| ATOM | 3186 | CB | VAL | A | 613 | 15.124 | −4.954 | 15.294 | 1.00 | 8.08 | | A | C |
| ANISOU | 3186 | CB | VAL | A | 613 | 979 | 1066 | 1025 | −31 | 61 | −16 | A | C |
| ATOM | 3188 | CG1 | VAL | A | 613 | 16.212 | −3.903 | 15.326 | 1.00 | 8.94 | | A | C |
| ANISOU | 3188 | CG1 | VAL | A | 613 | 1125 | 1147 | 1125 | −76 | 26 | 41 | A | C |
| ATOM | 3192 | CG2 | VAL | A | 613 | 15.295 | −5.871 | 14.104 | 1.00 | 7.92 | | A | C |
| ANISOU | 3192 | CG2 | VAL | A | 613 | 974 | 918 | 1114 | 56 | −26 | 54 | A | C |
| ATOM | 3196 | C | VAL | A | 613 | 14.924 | −4.850 | 17.811 | 1.00 | 7.57 | | A | C |
| ANISOU | 3196 | C | VAL | A | 613 | 913 | 1072 | 891 | −49 | 4 | 17 | A | C |
| ATOM | 3197 | O | VAL | A | 613 | 15.867 | −4.477 | 18.469 | 1.00 | 8.37 | | A | O |
| ANISOU | 3197 | O | VAL | A | 613 | 960 | 1207 | 1013 | −104 | −33 | 18 | A | O |
| ATOM | 3198 | N | CYS | A | 614 | 13.677 | −4.522 | 18.103 | 1.00 | 8.43 | | A | N |
| ANISOU | 3198 | N | CYS | A | 614 | 1052 | 1065 | 1085 | 16 | −28 | −41 | A | N |
| ATOM | 3200 | CA | CYS | A | 614 | 13.374 | −3.718 | 19.264 | 1.00 | 8.40 | | A | C |
| ANISOU | 3200 | CA | CYS | A | 614 | 1021 | 1112 | 1056 | 36 | 58 | −15 | A | C |
| ATOM | 3202 | CB | CYS | A | 614 | 11.865 | −3.464 | 19.290 | 1.00 | 9.06 | | A | C |
| ANISOU | 3202 | CB | CYS | A | 614 | 1089 | 1165 | 1187 | 64 | 61 | 30 | A | C |
| ATOM | 3205 | SG | CYS | A | 614 | 11.324 | −2.366 | 20.621 | 1.00 | 11.65 | | A | S |
| ANISOU | 3205 | SG | CYS | A | 614 | 1381 | 1541 | 1503 | 381 | −28 | −304 | A | S |
| ATOM | 3206 | C | CYS | A | 614 | 13.867 | −4.397 | 20.564 | 1.00 | 8.29 | | A | C |
| ANISOU | 3206 | C | CYS | A | 614 | 1023 | 1002 | 1125 | −25 | 30 | −23 | A | C |
| ATOM | 3207 | O | CYS | A | 614 | 14.461 | −3.744 | 21.429 | 1.00 | 9.16 | | A | O |
| ANISOU | 3207 | O | CYS | A | 614 | 1032 | 1308 | 1138 | 27 | −9 | −4 | A | O |
| ATOM | 3208 | N | MET | A | 615 | 13.654 | −5.708 | 20.688 | 1.00 | 8.27 | | A | N |
| ANISOU | 3208 | N | MET | A | 615 | 994 | 985 | 1163 | −60 | 3 | 92 | A | N |
| ATOM | 3210 | CA | MET | A | 615 | 14.185 | −6.416 | 21.852 | 1.00 | 8.60 | | A | C |
| ANISOU | 3210 | CA | MET | A | 615 | 1117 | 1096 | 1054 | 24 | 48 | 18 | A | C |
| ATOM | 3212 | CB | MET | A | 615 | 13.794 | −7.892 | 21.841 | 1.00 | 9.35 | | A | C |
| ANISOU | 3212 | CB | MET | A | 615 | 1195 | 1150 | 1207 | −33 | −6 | 0 | A | C |
| ATOM | 3215 | CG | MET | A | 615 | 12.336 | −8.165 | 22.031 | 1.00 | 10.66 | | A | C |
| ANISOU | 3215 | CG | MET | A | 615 | 1304 | 1337 | 1409 | 0 | 67 | 118 | A | C |
| ATOM | 3218 | SD | MET | A | 615 | 11.989 | −9.904 | 22.526 | 1.00 | 13.11 | | A | S |
| ANISOU | 3218 | SD | MET | A | 615 | 1496 | 1477 | 2007 | −159 | 83 | 378 | A | S |
| ATOM | 3219 | CE | MET | A | 615 | 12.205 | −10.770 | 20.949 | 1.00 | 14.46 | | A | C |
| ANISOU | 3219 | CE | MET | A | 615 | 1773 | 1582 | 2138 | −87 | 155 | 76 | A | C |
| ATOM | 3223 | C | MET | A | 615 | 15.691 | −6.323 | 21.906 | 1.00 | 8.07 | | A | C |
| ANISOU | 3223 | C | MET | A | 615 | 1079 | 956 | 1029 | 55 | 23 | 41 | A | C |
| ATOM | 3224 | O | MET | A | 615 | 16.261 | −6.134 | 22.977 | 1.00 | 8.96 | | A | O |
| ANISOU | 3224 | O | MET | A | 615 | 1292 | 1100 | 1012 | 121 | 83 | 13 | A | O |
| ATOM | 3225 | N | TRP | A | 616 | 16.349 | −6.428 | 20.748 | 1.00 | 8.09 | | A | N |
| ANISOU | 3225 | N | TRP | A | 616 | 1112 | 1019 | 939 | 114 | −49 | 27 | A | N |
| ATOM | 3227 | CA | TRP | A | 616 | 17.802 | −6.264 | 20.692 | 1.00 | 8.17 | | A | C |
| ANISOU | 3227 | CA | TRP | A | 616 | 1131 | 1004 | 966 | 75 | −26 | −3 | A | C |
| ATOM | 3229 | CB | TRP | A | 616 | 18.327 | −6.540 | 19.276 | 1.00 | 8.03 | | A | C |
| ANISOU | 3229 | CB | TRP | A | 616 | 1125 | 939 | 985 | 85 | 20 | 19 | A | C |
| ATOM | 3232 | CG | TRP | A | 616 | 19.790 | −6.449 | 19.151 | 1.00 | 7.37 | | A | C |
| ANISOU | 3232 | CG | TRP | A | 616 | 1017 | 909 | 873 | 22 | −84 | −42 | A | C |
| ATOM | 3233 | CD1 | TRP | A | 616 | 20.648 | −7.482 | 19.193 | 1.00 | 8.05 | | A | C |
| ANISOU | 3233 | CD1 | TRP | A | 616 | 1119 | 1061 | 877 | 129 | 70 | 68 | A | C |
| ATOM | 3235 | NE1 | TRP | A | 616 | 21.940 | −7.044 | 19.044 | 1.00 | 8.55 | | A | N |
| ANISOU | 3235 | NE1 | TRP | A | 616 | 1074 | 1128 | 1046 | 63 | 24 | 22 | A | N |
| ATOM | 3237 | CE2 | TRP | A | 616 | 21.946 | −5.686 | 18.895 | 1.00 | 8.19 | | A | C |
| ANISOU | 3237 | CE2 | TRP | A | 616 | 1105 | 1016 | 988 | 9 | −27 | 3 | A | C |
| ATOM | 3238 | CD2 | TRP | A | 616 | 20.592 | −5.268 | 18.938 | 1.00 | 8.11 | | A | C |
| ANISOU | 3238 | CD2 | TRP | A | 616 | 1033 | 993 | 1056 | 27 | 43 | 15 | A | C |
| ATOM | 3239 | CE3 | TRP | A | 616 | 20.306 | −3.896 | 18.799 | 1.00 | 7.66 | | A | C |
| ANISOU | 3239 | CE3 | TRP | A | 616 | 968 | 1061 | 880 | 103 | 17 | −50 | A | C |
| ATOM | 3241 | CZ3 | TRP | A | 616 | 21.347 | −3.004 | 18.624 | 1.00 | 9.44 | | A | C |
| ANISOU | 3241 | CZ3 | TRP | A | 616 | 1356 | 1158 | 1072 | −34 | 44 | −11 | A | C |
| ATOM | 3243 | CH2 | TRP | A | 616 | 22.699 | −3.455 | 18.561 | 1.00 | 9.10 | | A | C |
| ANISOU | 3243 | CH2 | TRP | A | 616 | 1173 | 1093 | 1191 | −100 | 118 | 41 | A | C |
| ATOM | 3245 | CZ2 | TRP | A | 616 | 23.013 | −4.792 | 18.688 | 1.00 | 7.47 | | A | C |
| ANISOU | 3245 | CZ2 | TRP | A | 616 | 796 | 1171 | 872 | 140 | −57 | 103 | A | C |
| ATOM | 3247 | C | TRP | A | 616 | 18.191 | −4.868 | 21.202 | 1.00 | 8.49 | | A | C |
| ANISOU | 3247 | C | TRP | A | 616 | 1121 | 1094 | 1010 | 27 | 0 | −16 | A | C |
| ATOM | 3248 | O | TRP | A | 616 | 19.128 | −4.740 | 22.001 | 1.00 | 8.40 | | A | O |
| ANISOU | 3248 | O | TRP | A | 616 | 1102 | 1097 | 992 | 48 | −62 | −44 | A | O |
| ATOM | 3249 | N | GLU | A | 617 | 17.446 | −3.846 | 20.808 | 1.00 | 8.56 | | A | N |
| ANISOU | 3249 | N | GLU | A | 617 | 1104 | 1176 | 972 | 26 | 55 | −50 | A | N |
| ATOM | 3251 | CA | GLU | A | 617 | 17.707 | −2.480 | 21.291 | 1.00 | 8.72 | | A | C |
| ANISOU | 3251 | CA | GLU | A | 617 | 1138 | 1122 | 1049 | −19 | 34 | −30 | A | C |
| ATOM | 3253 | CB | GLU | A | 617 | 16.741 | −1.474 | 20.683 | 1.00 | 10.12 | | A | C |
| ANISOU | 3253 | CB | GLU | A | 617 | 1364 | 1194 | 1286 | 37 | 13 | −31 | A | C |
| ATOM | 3256 | CG | GLU | A | 617 | 16.935 | −1.149 | 19.226 | 1.00 | 10.89 | | A | C |
| ANISOU | 3256 | CG | GLU | A | 617 | 1363 | 1413 | 1358 | 41 | 5 | −6 | A | C |
| ATOM | 3259 | CD | GLU | A | 617 | 15.885 | −0.160 | 18.752 | 1.00 | 10.67 | | A | C |
| ANISOU | 3259 | CD | GLU | A | 617 | 1444 | 1309 | 1300 | −64 | −98 | −28 | A | C |
| ATOM | 3260 | OE1 | GLU | A | 617 | 14.700 | −0.467 | 18.885 | 1.00 | 12.97 | | A | O |
| ANISOU | 3260 | OE1 | GLU | A | 617 | 1515 | 1667 | 1743 | 56 | 0 | −36 | A | O |
| ATOM | 3261 | OE2 | GLU | A | 617 | 16.235 | 0.929 | 18.273 | 1.00 | 13.44 | | A | O |
| ANISOU | 3261 | OE2 | GLU | A | 617 | 2111 | 1387 | 1607 | 143 | 94 | 261 | A | O |
| ATOM | 3262 | C | GLU | A | 617 | 17.546 | −2.389 | 22.804 | 1.00 | 8.46 | | A | C |
| ANISOU | 3262 | C | GLU | A | 617 | 1032 | 1175 | 1004 | −33 | 47 | −43 | A | C |
| ATOM | 3263 | O | GLU | A | 617 | 18.366 | −1.773 | 23.498 | 1.00 | 8.69 | | A | O |
| ANISOU | 3263 | O | GLU | A | 617 | 1089 | 1238 | 973 | −65 | 19 | −75 | A | O |
| ATOM | 3264 | N | ILE | A | 618 | 16.478 | −2.991 | 23.316 | 1.00 | 7.97 | | A | N |
| ANISOU | 3264 | N | ILE | A | 618 | 1115 | 965 | 946 | −34 | 38 | −41 | A | N |
| ATOM | 3266 | CA | ILE | A | 618 | 16.230 | −2.950 | 24.766 | 1.00 | 7.93 | | A | C |
| ANISOU | 3266 | CA | ILE | A | 618 | 1051 | 1005 | 955 | 76 | 27 | 30 | A | C |
| ATOM | 3268 | CB | ILE | A | 618 | 14.879 | −3.602 | 25.083 | 1.00 | 8.25 | | A | C |
| ANISOU | 3268 | CB | ILE | A | 618 | 1131 | 1002 | 999 | 60 | 26 | 0 | A | C |
| ATOM | 3270 | CG1 | ILE | A | 618 | 13.748 | −2.752 | 24.464 | 1.00 | 8.51 | | A | C |
| ANISOU | 3270 | CG1 | ILE | A | 618 | 1117 | 1100 | 1015 | 5 | 58 | 97 | A | C |
| ATOM | 3273 | CD1 | ILE | A | 618 | 12.366 | −3.346 | 24.595 | 1.00 | 9.45 | | A | C |
| ANISOU | 3273 | CD1 | ILE | A | 618 | 1179 | 1134 | 1277 | 53 | 148 | 10 | A | C |
| ATOM | 3277 | CG2 | ILE | A | 618 | 14.706 | −3.777 | 26.605 | 1.00 | 8.97 | | A | C |
| ANISOU | 3277 | CG2 | ILE | A | 618 | 1159 | 1211 | 1036 | 90 | 40 | 53 | A | C |
| ATOM | 3281 | C | ILE | A | 618 | 17.389 | −3.599 | 25.542 | 1.00 | 8.12 | | A | C |
| ANISOU | 3281 | C | ILE | A | 618 | 994 | 1054 | 1038 | −19 | −3 | −40 | A | C |
| ATOM | 3282 | O | ILE | A | 618 | 17.924 | −3.029 | 26.507 | 1.00 | 8.54 | | A | O |
| ANISOU | 3282 | O | ILE | A | 618 | 1122 | 1077 | 1044 | −1 | 97 | −32 | A | O |
| ATOM | 3283 | N | LEU | A | 619 | 17.755 | −4.815 | 25.110 | 1.00 | 8.06 | | A | N |
| ANISOU | 3283 | N | LEU | A | 619 | 1022 | 1042 | 997 | 56 | −64 | 4 | A | N |
| ATOM | 3285 | CA | LEU | A | 619 | 18.817 | −5.570 | 25.803 | 1.00 | 9.30 | | A | C |
| ANISOU | 3285 | CA | LEU | A | 619 | 1150 | 1164 | 1217 | 24 | −21 | 18 | A | C |
| ATOM | 3287 | CB | LEU | A | 619 | 18.771 | −7.042 | 25.366 | 1.00 | 9.66 | | A | C |
| ANISOU | 3287 | CB | LEU | A | 619 | 1250 | 1206 | 1214 | 38 | −46 | −39 | A | C |
| ATOM | 3290 | CG | LEU | A | 619 | 17.852 | −7.985 | 26.156 | 1.00 | 10.45 | | A | C |
| ANISOU | 3290 | CG | LEU | A | 619 | 1313 | 1244 | 1411 | 25 | 23 | −73 | A | C |
| ATOM | 3292 | CD1 | LEU | A | 619 | 18.316 | −8.193 | 27.599 | 1.00 | 11.33 | | A | C |
| ANISOU | 3292 | CD1 | LEU | A | 619 | 1599 | 1208 | 1496 | 98 | 55 | 23 | A | C |
| ATOM | 3296 | CD2 | LEU | A | 619 | 16.378 | −7.523 | 26.133 | 1.00 | 10.51 | | A | C |
| ANISOU | 3296 | CD2 | LEU | A | 619 | 1328 | 1153 | 1509 | 53 | −27 | 19 | A | C |
| ATOM | 3300 | C | LEU | A | 619 | 20.193 | −4.965 | 25.575 | 1.00 | 8.94 | | A | C |
| ANISOU | 3300 | C | LEU | A | 619 | 1136 | 1142 | 1118 | 63 | −19 | −31 | A | C |
| ATOM | 3301 | O | LEU | A | 619 | 21.121 | −5.297 | 26.290 | 1.00 | 10.08 | | A | O |
| ANISOU | 3301 | O | LEU | A | 619 | 1381 | 1287 | 1162 | 56 | −119 | −2 | A | O |
| ATOM | 3302 | N | SER | A | 620 | 20.296 | −4.033 | 24.631 | 1.00 | 8.88 | | A | N |
| ANISOU | 3302 | N | SER | A | 620 | 1103 | 1151 | 1117 | −3 | −50 | 13 | A | N |
| ATOM | 3304 | CA | SER | A | 620 | 21.505 | −3.261 | 24.373 | 1.00 | 9.66 | | A | C |
| ANISOU | 3304 | CA | SER | A | 620 | 1202 | 1184 | 1282 | 53 | 8 | 10 | A | C |
| ATOM | 3306 | CB | SER | A | 620 | 21.728 | −3.183 | 22.869 | 1.00 | 9.29 | | A | C |
| ANISOU | 3306 | CB | SER | A | 620 | 1200 | 1104 | 1224 | 49 | −5 | −98 | A | C |
| ATOM | 3309 | OG | SER | A | 620 | 21.807 | −4.470 | 22.292 | 1.00 | 10.72 | | A | O |
| ANISOU | 3309 | OG | SER | A | 620 | 1387 | 1338 | 1346 | 65 | −36 | −323 | A | O |
| ATOM | 3311 | C | SER | A | 620 | 21.456 | −1.832 | 24.937 | 1.00 | 9.21 | | A | C |
| ANISOU | 3311 | C | SER | A | 620 | 1174 | 1196 | 1129 | −50 | 35 | −27 | A | C |
| ATOM | 3312 | O | SER | A | 620 | 22.307 | −1.008 | 24.608 | 1.00 | 11.06 | | A | O |
| ANISOU | 3312 | O | SER | A | 620 | 1474 | 1382 | 1344 | −139 | 85 | −4 | A | O |
| ATOM | 3313 | N | PHE | A | 621 | 20.462 | −1.529 | 25.771 | 1.00 | 8.95 | | A | N |
| ANISOU | 3313 | N | PHE | A | 621 | 1114 | 1132 | 1155 | −23 | 27 | −28 | A | N |
| ATOM | 3315 | CA | PHE | A | 621 | 20.337 | −0.218 | 26.387 | 1.00 | 9.65 | | A | C |
| ANISOU | 3315 | CA | PHE | A | 621 | 1236 | 1282 | 1145 | 46 | 20 | −98 | A | C |
| ATOM | 3317 | CB | PHE | A | 621 | 21.468 | 0.011 | 27.414 | 1.00 | 10.29 | | A | C |
| ANISOU | 3317 | CB | PHE | A | 621 | 1288 | 1375 | 1245 | 0 | 3 | −64 | A | C |
| ATOM | 3320 | CG | PHE | A | 621 | 21.477 | −1.003 | 28.516 | 1.00 | 9.62 | | A | C |
| ANISOU | 3320 | CG | PHE | A | 621 | 1337 | 1174 | 1142 | −58 | −37 | −88 | A | C |
| ATOM | 3321 | CD1 | PHE | A | 621 | 20.646 | −0.866 | 29.598 | 1.00 | 11.01 | | A | C |
| ANISOU | 3321 | CD1 | PHE | A | 621 | 1456 | 1403 | 1323 | 1 | 25 | −48 | A | C |
| ATOM | 3323 | CE1 | PHE | A | 621 | 20.632 | −1.815 | 30.628 | 1.00 | 11.66 | | A | C |
| ANISOU | 3323 | CE1 | PHE | A | 621 | 1557 | 1483 | 1388 | 11 | 4 | 6 | A | C |
| ATOM | 3325 | CZ | PHE | A | 621 | 21.478 | −2.876 | 30.576 | 1.00 | 12.92 | | A | C |
| ANISOU | 3325 | CZ | PHE | A | 621 | 1757 | 1692 | 1458 | 113 | 121 | 56 | A | C |
| ATOM | 3327 | CE2 | PHE | A | 621 | 22.325 | −3.025 | 29.477 | 1.00 | 14.15 | | A | C |
| ANISOU | 3327 | CE2 | PHE | A | 621 | 1903 | 1798 | 1672 | 126 | 166 | 30 | A | C |
| ATOM | 3329 | CD2 | PHE | A | 621 | 22.320 | −2.086 | 28.478 | 1.00 | 11.25 | | A | C |
| ANISOU | 3329 | CD2 | PHE | A | 621 | 1501 | 1547 | 1223 | 98 | 92 | −83 | A | C |
| ATOM | 3331 | C | PHE | A | 621 | 20.241 | 0.921 | 25.373 | 1.00 | 10.39 | | A | C |
| ANISOU | 3331 | C | PHE | A | 621 | 1360 | 1302 | 1286 | 22 | −4 | −79 | A | C |
| ATOM | 3332 | O | PHE | A | 621 | 20.786 | 2.011 | 25.569 | 1.00 | 11.34 | | A | O |
| ANISOU | 3332 | O | PHE | A | 621 | 1621 | 1375 | 1311 | 32 | −13 | −117 | A | O |
| ATOM | 3333 | N | GLY | A | 622 | 19.522 | 0.651 | 24.285 | 1.00 | 11.10 | | A | N |
| ANISOU | 3333 | N | GLY | A | 622 | 1512 | 1375 | 1328 | 42 | −63 | −81 | A | N |
| ATOM | 3335 | CA | GLY | A | 622 | 19.193 | 1.662 | 23.303 | 1.00 | 11.22 | | A | C |
| ANISOU | 3335 | CA | GLY | A | 622 | 1468 | 1417 | 1376 | 66 | 4 | −22 | A | C |
| ATOM | 3338 | C | GLY | A | 622 | 20.124 | 1.861 | 22.126 | 1.00 | 12.73 | | A | C |
| ANISOU | 3338 | C | GLY | A | 622 | 1686 | 1597 | 1551 | 18 | 20 | −39 | A | C |
| ATOM | 3339 | O | GLY | A | 622 | 19.920 | 2.776 | 21.338 | 1.00 | 14.16 | | A | O |
| ANISOU | 3339 | O | GLY | A | 622 | 1927 | 1912 | 1540 | 219 | −37 | −18 | A | O |
| ATOM | 3340 | N | LYS | A | 623 | 21.153 | 1.030 | 21.998 | 1.00 | 12.99 | | A | N |
| ANISOU | 3340 | N | LYS | A | 623 | 1681 | 1643 | 1610 | 69 | −29 | −17 | A | N |
| ATOM | 3342 | CA | LYS | A | 623 | 22.031 | 1.135 | 20.830 | 1.00 | 14.46 | | A | C |
| ANISOU | 3342 | CA | LYS | A | 623 | 1800 | 1899 | 1795 | 0 | −6 | −28 | A | C |
| ATOM | 3344 | CB | LYS | A | 623 | 23.171 | 0.133 | 20.897 | 1.00 | 16.42 | | A | C |
| ANISOU | 3344 | CB | LYS | A | 623 | 2096 | 2093 | 2050 | 76 | 2 | 5 | A | C |
| ATOM | 3347 | CG | LYS | A | 623 | 24.168 | 0.387 | 22.051 | 1.00 | 19.39 | | A | C |
| ANISOU | 3347 | CG | LYS | A | 623 | 2346 | 2627 | 2395 | 31 | −71 | −13 | A | C |
| ATOM | 3350 | CD | LYS | A | 623 | 24.349 | 1.876 | 22.432 | 1.00 | 23.86 | | A | C |
| ANISOU | 3350 | CD | LYS | A | 623 | 3111 | 2953 | 2998 | −20 | −35 | −29 | A | C |
| ATOM | 3353 | CE | LYS | A | 623 | 25.567 | 2.118 | 23.318 | 1.00 | 25.46 | | A | C |
| ANISOU | 3353 | CE | LYS | A | 623 | 3218 | 3278 | 3177 | 8 | −71 | 33 | A | C |
| ATOM | 3356 | NZ | LYS | A | 623 | 25.748 | 3.571 | 23.628 | 1.00 | 27.42 | | A | N |
| ANISOU | 3356 | NZ | LYS | A | 623 | 3624 | 3417 | 3377 | −57 | −46 | −49 | A | N |
| ATOM | 3360 | C | LYS | A | 623 | 21.255 | 0.917 | 19.534 | 1.00 | 13.13 | | A | C |
| ANISOU | 3360 | C | LYS | A | 623 | 1680 | 1666 | 1643 | 49 | 26 | −50 | A | C |
| ATOM | 3361 | O | LYS | A | 623 | 20.255 | 0.223 | 19.513 | 1.00 | 12.72 | | A | O |
| ANISOU | 3361 | O | LYS | A | 623 | 1567 | 1670 | 1594 | 15 | 118 | −135 | A | O |
| ATOM | 3362 | N | GLN | A | 624 | 21.722 | 1.549 | 18.473 | 1.00 | 12.29 | | A | N |
| ANISOU | 3362 | N | GLN | A | 624 | 1579 | 1448 | 1640 | 9 | 21 | −24 | A | N |
| ATOM | 3364 | CA | GLN | A | 624 | 21.102 | 1.440 | 17.176 | 1.00 | 12.12 | | A | C |
| ANISOU | 3364 | CA | GLN | A | 624 | 1546 | 1487 | 1573 | 20 | 19 | −7 | A | C |
| ATOM | 3366 | CB | GLN | A | 624 | 21.541 | 2.586 | 16.243 | 1.00 | 13.32 | | A | C |
| ANISOU | 3366 | CB | GLN | A | 624 | 1719 | 1539 | 1803 | 31 | 66 | 15 | A | C |
| ATOM | 3369 | CG | GLN | A | 624 | 20.909 | 2.482 | 14.840 | 1.00 | 18.07 | | A | C |
| ANISOU | 3369 | CG | GLN | A | 624 | 2357 | 2269 | 2240 | 73 | −72 | −62 | A | C |
| ATOM | 3372 | CD | GLN | A | 624 | 21.509 | 3.377 | 13.743 | 1.00 | 22.31 | | A | C |
| ANISOU | 3372 | CD | GLN | A | 624 | 2878 | 2762 | 2837 | −40 | 63 | 88 | A | C |
| ATOM | 3373 | OE1 | GLN | A | 624 | 22.588 | 3.095 | 13.196 | 1.00 | 24.31 | | A | O |
| ANISOU | 3373 | OE1 | GLN | A | 624 | 2960 | 3051 | 3224 | 92 | 9 | 132 | A | O |
| ATOM | 3374 | NE2 | GLN | A | 624 | 20.752 | 4.398 | 13.348 | 1.00 | 26.33 | | A | N |
| ANISOU | 3374 | NE2 | GLN | A | 624 | 3465 | 3053 | 3484 | 164 | −3 | 82 | A | N |
| ATOM | 3377 | C | GLN | A | 624 | 21.489 | 0.123 | 16.542 | 1.00 | 10.73 | | A | C |
| ANISOU | 3377 | C | GLN | A | 624 | 1339 | 1325 | 1410 | −9 | −7 | 41 | A | C |
| ATOM | 3378 | O | GLN | A | 624 | 22.681 | −0.206 | 16.488 | 1.00 | 10.29 | | A | O |
| ANISOU | 3378 | O | GLN | A | 624 | 1224 | 1167 | 1518 | −91 | 43 | −37 | A | O |
| ATOM | 3379 | N | PRO | A | 625 | 20.516 | −0.603 | 16.000 | 1.00 | 9.27 | | A | N |
| ANISOU | 3379 | N | PRO | A | 625 | 1193 | 1126 | 1200 | −19 | 20 | −26 | A | N |
| ATOM | 3380 | CA | PRO | A | 625 | 20.831 | −1.847 | 15.293 | 1.00 | 9.00 | | A | C |
| ANISOU | 3380 | CA | PRO | A | 625 | 1099 | 1095 | 1224 | 57 | 50 | 15 | A | C |
| ATOM | 3382 | CB | PRO | A | 625 | 19.456 | −2.402 | 14.917 | 1.00 | 8.99 | | A | C |
| ANISOU | 3382 | CB | PRO | A | 625 | 1186 | 1082 | 1145 | 35 | 24 | 46 | A | C |
| ATOM | 3385 | CG | PRO | A | 625 | 18.570 | −1.210 | 14.867 | 1.00 | 9.14 | | A | C |
| ANISOU | 3385 | CG | PRO | A | 625 | 1226 | 1066 | 1180 | 86 | 122 | 46 | A | C |
| ATOM | 3388 | CD | PRO | A | 625 | 19.069 | −0.317 | 15.969 | 1.00 | 9.56 | | A | C |
| ANISOU | 3388 | CD | PRO | A | 625 | 1275 | 1169 | 1187 | 34 | 53 | 105 | A | C |
| ATOM | 3391 | C | PRO | A | 625 | 21.685 | −1.542 | 14.067 | 1.00 | 8.92 | | A | C |
| ANISOU | 3391 | C | PRO | A | 625 | 1173 | 1016 | 1200 | 25 | 48 | 54 | A | C |
| ATOM | 3392 | O | PRO | A | 625 | 21.435 | −0.568 | 13.358 | 1.00 | 9.28 | | A | O |
| ANISOU | 3392 | O | PRO | A | 625 | 1224 | 1087 | 1215 | 71 | 94 | 122 | A | O |
| ATOM | 3393 | N | PHE | A | 626 | 22.701 | −2.370 | 13.815 | 1.00 | 7.60 | | A | N |
| ANISOU | 3393 | N | PHE | A | 626 | 936 | 903 | 1048 | 19 | −50 | 11 | A | N |
| ATOM | 3395 | CA | PHE | A | 626 | 23.534 | −2.225 | 12.629 | 1.00 | 7.73 | | A | C |
| ANISOU | 3395 | CA | PHE | A | 626 | 1076 | 877 | 982 | 12 | −48 | 21 | A | C |
| ATOM | 3397 | CB | PHE | A | 626 | 22.737 | −2.566 | 11.356 | 1.00 | 7.40 | | A | C |
| ANISOU | 3397 | CB | PHE | A | 626 | 1034 | 884 | 894 | −3 | 5 | −12 | A | C |
| ATOM | 3400 | CG | PHE | A | 626 | 22.329 | −4.021 | 11.255 | 1.00 | 8.09 | | A | C |
| ANISOU | 3400 | CG | PHE | A | 626 | 1041 | 1024 | 1008 | −86 | −2 | −46 | A | C |
| ATOM | 3401 | CD1 | PHE | A | 626 | 23.294 | −5.024 | 11.215 | 1.00 | 7.67 | | A | C |
| ANISOU | 3401 | CD1 | PHE | A | 626 | 1036 | 890 | 988 | −162 | −239 | 27 | A | C |
| ATOM | 3403 | CE1 | PHE | A | 626 | 22.924 | −6.362 | 11.159 | 1.00 | 8.27 | | A | C |
| ANISOU | 3403 | CE1 | PHE | A | 626 | 1141 | 784 | 1216 | 93 | −7 | −18 | A | C |
| ATOM | 3405 | CZ | PHE | A | 626 | 21.587 | −6.709 | 11.106 | 1.00 | 8.76 | | A | C |
| ANISOU | 3405 | CZ | PHE | A | 626 | 1255 | 969 | 1101 | −52 | −21 | −137 | A | C |
| ATOM | 3407 | CE2 | PHE | A | 626 | 20.608 | −5.726 | 11.146 | 1.00 | 7.41 | | A | C |
| ANISOU | 3407 | CE2 | PHE | A | 626 | 984 | 1007 | 823 | −114 | 46 | 0 | A | C |
| ATOM | 3409 | CD2 | PHE | A | 626 | 20.983 | −4.378 | 11.219 | 1.00 | 8.31 | | A | C |
| ANISOU | 3409 | CD2 | PHE | A | 626 | 1055 | 1051 | 1049 | −57 | 4 | 28 | A | C |
| ATOM | 3411 | C | PHE | A | 626 | 24.146 | −0.827 | 12.547 | 1.00 | 8.26 | | A | C |
| ANISOU | 3411 | C | PHE | A | 626 | 1166 | 926 | 1043 | 37 | −49 | 14 | A | C |
| ATOM | 3412 | O | PHE | A | 626 | 24.344 | −0.279 | 11.477 | 1.00 | 8.99 | | A | O |
| ANISOU | 3412 | O | PHE | A | 626 | 1258 | 970 | 1188 | 51 | −9 | −20 | A | O |
| ATOM | 3413 | N | PHE | A | 627 | 24.528 | −0.292 | 13.699 | 1.00 | 8.24 | | A | N |
| ANISOU | 3413 | N | PHE | A | 627 | 1259 | 876 | 995 | −4 | −74 | 26 | A | N |
| ATOM | 3415 | CA | PHE | A | 627 | 25.174 | 1.024 | 13.707 | 1.00 | 9.11 | | A | C |
| ANISOU | 3415 | CA | PHE | A | 627 | 1240 | 1060 | 1159 | −58 | −25 | −17 | A | C |
| ATOM | 3417 | CB | PHE | A | 627 | 25.424 | 1.511 | 15.140 | 1.00 | 9.32 | | A | C |
| ANISOU | 3417 | CB | PHE | A | 627 | 1293 | 1146 | 1103 | −79 | −39 | −37 | A | C |
| ATOM | 3420 | CG | PHE | A | 627 | 26.369 | 0.645 | 15.944 | 1.00 | 9.19 | | A | C |
| ANISOU | 3420 | CG | PHE | A | 627 | 1223 | 1095 | 1172 | −86 | −26 | −71 | A | C |
| ATOM | 3421 | CD1 | PHE | A | 627 | 27.735 | 0.787 | 15.834 | 1.00 | 10.25 | | A | C |
| ANISOU | 3421 | CD1 | PHE | A | 627 | 1241 | 1227 | 1427 | −6 | −56 | −130 | A | C |
| ATOM | 3423 | CE1 | PHE | A | 627 | 28.598 | −0.008 | 16.569 | 1.00 | 9.28 | | A | C |
| ANISOU | 3423 | CE1 | PHE | A | 627 | 1125 | 1270 | 1131 | −139 | −83 | −7 | A | C |
| ATOM | 3425 | CZ | PHE | A | 627 | 28.106 | −0.937 | 17.430 | 1.00 | 10.17 | | A | C |
| ANISOU | 3425 | CZ | PHE | A | 627 | 1354 | 1332 | 1178 | 51 | −91 | 64 | A | C |
| ATOM | 3427 | CE2 | PHE | A | 627 | 26.740 | −1.080 | 17.553 | 1.00 | 10.61 | | A | C |
| ANISOU | 3427 | CE2 | PHE | A | 627 | 1444 | 1401 | 1183 | −72 | −11 | −72 | A | C |
| ATOM | 3429 | CD2 | PHE | A | 627 | 25.893 | −0.299 | 16.833 | 1.00 | 8.87 | | A | C |
| ANISOU | 3429 | CD2 | PHE | A | 627 | 1305 | 1018 | 1045 | −100 | 9 | −208 | A | C |
| ATOM | 3431 | C | PHE | A | 627 | 26.456 | 1.077 | 12.892 | 1.00 | 10.08 | | A | C |
| ANISOU | 3431 | C | PHE | A | 627 | 1346 | 1201 | 1281 | −68 | 0 | 23 | A | C |
| ATOM | 3432 | O | PHE | A | 627 | 26.887 | 2.177 | 12.510 | 1.00 | 11.98 | | A | O |
| ANISOU | 3432 | O | PHE | A | 627 | 1683 | 1284 | 1583 | −151 | 37 | 95 | A | O |
| ATOM | 3433 | N | TRP | A | 628 | 27.053 | −0.093 | 12.649 | 1.00 | 10.14 | | A | N |
| ANISOU | 3433 | N | TRP | A | 628 | 1228 | 1310 | 1315 | −37 | −3 | −3 | A | N |
| ATOM | 3435 | CA | TRP | A | 628 | 28.347 | −0.244 | 11.983 | 1.00 | 11.50 | | A | C |
| ANISOU | 3435 | CA | TRP | A | 628 | 1418 | 1502 | 1446 | −38 | 19 | 8 | A | C |
| ATOM | 3437 | CB | TRP | A | 628 | 29.083 | −1.495 | 12.512 | 1.00 | 11.81 | | A | C |
| ANISOU | 3437 | CB | TRP | A | 628 | 1473 | 1562 | 1452 | 21 | −8 | −3 | A | C |
| ATOM | 3440 | CG | TRP | A | 628 | 28.265 | −2.820 | 12.467 | 1.00 | 11.23 | | A | C |
| ANISOU | 3440 | CG | TRP | A | 628 | 1405 | 1410 | 1451 | 93 | −65 | −64 | A | C |
| ATOM | 3441 | CD1 | TRP | A | 628 | 28.293 | −3.793 | 11.476 | 1.00 | 11.11 | | A | C |
| ANISOU | 3441 | CD1 | TRP | A | 628 | 1339 | 1477 | 1406 | 170 | −262 | −42 | A | C |
| ATOM | 3443 | NE1 | TRP | A | 628 | 27.423 | −4.809 | 11.789 | 1.00 | 12.58 | | A | N |
| ANISOU | 3443 | NE2 | TRP | A | 628 | 1741 | 1422 | 1616 | 220 | −272 | −9 | A | N |
| ATOM | 3445 | CE2 | TRP | A | 628 | 26.795 | −4.531 | 12.966 | 1.00 | 11.44 | | A | C |
| ANISOU | 3445 | CE2 | TRP | A | 628 | 1445 | 1326 | 1572 | 113 | −268 | 136 | A | C |
| ATOM | 3446 | CD2 | TRP | A | 628 | 27.306 | −3.277 | 13.427 | 1.00 | 10.60 | | A | C |
| ANISOU | 3446 | CD2 | TRP | A | 628 | 1462 | 1248 | 1315 | 0 | −261 | 72 | A | C |
| ATOM | 3447 | CE3 | TRP | A | 628 | 26.816 | −2.770 | 14.624 | 1.00 | 10.04 | | A | C |
| ANISOU | 3447 | CE3 | TRP | A | 628 | 1218 | 1287 | 1308 | 100 | −209 | 58 | A | C |
| ATOM | 3449 | CZ3 | TRP | A | 628 | 25.850 | −3.499 | 15.329 | 1.00 | 11.14 | | A | C |
| ANISOU | 3449 | CZ3 | TRP | A | 628 | 1512 | 1390 | 1329 | −80 | −227 | −54 | A | C |
| ATOM | 3451 | CH2 | TRP | A | 628 | 25.372 | −4.727 | 14.846 | 1.00 | 12.39 | | A | C |
| ANISOU | 3451 | CH2 | TRP | A | 628 | 1663 | 1370 | 1675 | −8 | −202 | −3 | A | C |
| ATOM | 3453 | CZ2 | TRP | A | 628 | 25.848 | −5.260 | 13.680 | 1.00 | 10.83 | | A | C |
| ANISOU | 3453 | CZ2 | TRP | A | 628 | 1406 | 1287 | 1419 | 60 | −245 | 50 | A | C |
| ATOM | 3455 | C | TRP | A | 628 | 28.219 | −0.323 | 10.460 | 1.00 | 12.25 | | A | C |
| ANISOU | 3455 | C | TRP | A | 628 | 1482 | 1674 | 1495 | −25 | 19 | 22 | A | C |
| ATOM | 3456 | O | TRP | A | 628 | 29.234 | −0.395 | 9.757 | 1.00 | 14.02 | | A | O |
| ANISOU | 3456 | O | TRP | A | 628 | 1642 | 2078 | 1606 | 7 | 63 | 11 | A | O |
| ATOM | 3457 | N | LEU | A | 629 | 26.980 | −0.302 | 9.964 | 1.00 | 12.51 | | A | N |
| ANISOU | 3457 | N | LEU | A | 629 | 1535 | 1705 | 1512 | −32 | −17 | 29 | A | N |
| ATOM | 3459 | CA | LEU | A | 629 | 26.669 | −0.380 | 8.536 | 1.00 | 12.79 | | A | C |
| ANISOU | 3459 | CA | LEU | A | 629 | 1617 | 1700 | 1542 | −32 | 11 | 7 | A | C |
| ATOM | 3461 | CB | LEU | A | 629 | 25.702 | −1.541 | 8.289 | 1.00 | 12.60 | | A | C |
| ANISOU | 3461 | CB | LEU | A | 629 | 1610 | 1647 | 1529 | −14 | −9 | −14 | A | C |
| ATOM | 3464 | CG | LEU | A | 629 | 26.137 | −2.945 | 8.678 | 1.00 | 12.33 | | A | C |
| ANISOU | 3464 | CG | LEU | A | 629 | 1646 | 1651 | 1386 | 14 | −97 | −1 | A | C |
| ATOM | 3466 | CD1 | LEU | A | 629 | 25.054 | −3.913 | 8.301 | 1.00 | 13.38 | | A | C |
| ANISOU | 3466 | CD1 | LEU | A | 629 | 1752 | 1716 | 1614 | −33 | −6 | 93 | A | C |
| ATOM | 3470 | CD2 | LEU | A | 629 | 27.422 | −3.323 | 8.009 | 1.00 | 13.37 | | A | C |
| ANISOU | 3470 | CD2 | LEU | A | 629 | 1713 | 1765 | 1599 | 56 | −77 | 0 | A | C |
| ATOM | 3474 | C | LEU | A | 629 | 25.978 | 0.881 | 8.034 | 1.00 | 13.68 | | A | C |
| ANISOU | 3474 | C | LEU | A | 629 | 1762 | 1761 | 1673 | 5 | 7 | 25 | A | C |
| ATOM | 3475 | O | LEU | A | 629 | 25.349 | 1.609 | 8.806 | 1.00 | 14.67 | | A | O |
| ANISOU | 3475 | O | LEU | A | 629 | 2007 | 1915 | 1652 | 56 | 17 | 146 | A | O |
| ATOM | 3476 | N | GLU | A | 630 | 26.047 | 1.100 | 6.723 | 1.00 | 15.20 | | A | N |
| ANISOU | 3476 | N | GLU | A | 630 | 1933 | 1972 | 1868 | −43 | 19 | 53 | A | N |
| ATOM | 3478 | CA | GLU | A | 630 | 25.195 | 2.078 | 6.050 | 1.00 | 15.90 | | A | C |
| ANISOU | 3478 | CA | GLU | A | 630 | 2047 | 2013 | 1981 | −18 | −32 | 34 | A | C |
| ATOM | 3480 | CB | GLU | A | 630 | 25.887 | 2.644 | 4.805 | 1.00 | 16.75 | | A | C |
| ANISOU | 3480 | CB | GLU | A | 630 | 2158 | 2129 | 2076 | −24 | 14 | 48 | A | C |
| ATOM | 3483 | CG | GLU | A | 630 | 27.081 | 3.540 | 5.068 | 1.00 | 19.59 | | A | C |
| ANISOU | 3483 | CG | GLU | A | 630 | 2476 | 2449 | 2518 | −38 | −51 | −29 | A | C |
| ATOM | 3486 | CD | GLU | A | 630 | 27.635 | 4.127 | 3.778 | 1.00 | 23.92 | | A | C |
| ANISOU | 3486 | CD | GLU | A | 630 | 3112 | 3047 | 2928 | −45 | 50 | 79 | A | C |
| ATOM | 3487 | OE1 | GLU | A | 630 | 28.396 | 3.432 | 3.073 | 1.00 | 25.66 | | A | O |
| ANISOU | 3487 | OE1 | GLU | A | 630 | 3327 | 3302 | 3119 | 51 | 180 | 61 | A | O |
| ATOM | 3488 | OE2 | GLU | A | 630 | 27.278 | 5.275 | 3.454 | 1.00 | 27.92 | | A | O |
| ANISOU | 3488 | OE2 | GLU | A | 630 | 3780 | 3258 | 3571 | 71 | −64 | 56 | A | O |
| ATOM | 3489 | C | GLU | A | 630 | 23.916 | 1.364 | 5.631 | 1.00 | 15.32 | | A | C |
| ANISOU | 3489 | C | GLU | A | 630 | 1983 | 1960 | 1875 | 0 | −57 | 56 | A | C |
| ATOM | 3490 | O | GLU | A | 630 | 23.947 | 0.155 | 5.409 | 1.00 | 14.36 | | A | O |
| ANISOU | 3490 | O | GLU | A | 630 | 1855 | 1857 | 1744 | −22 | −239 | 28 | A | O |
| ATOM | 3491 | N | ASN | A | 631 | 22.802 | 2.095 | 5.510 | 1.00 | 15.10 | | A | N |
| ANISOU | 3491 | N | ASN | A | 631 | 2014 | 1832 | 1889 | −11 | −36 | 32 | A | N |
| ATOM | 3493 | CA | ASN | A | 631 | 21.527 | 1.489 | 5.144 | 1.00 | 15.38 | | A | C |
| ANISOU | 3493 | CA | ASN | A | 631 | 2016 | 1916 | 1909 | 9 | −24 | 57 | A | C |
| ATOM | 3495 | CB | ASN | A | 631 | 20.452 | 2.554 | 4.851 | 1.00 | 15.89 | | A | C |
| ANISOU | 3495 | CB | ASN | A | 631 | 2019 | 2001 | 2014 | 42 | −89 | 39 | A | C |
| ATOM | 3498 | CG | ASN | A | 631 | 19.746 | 3.067 | 6.094 | 1.00 | 16.81 | | A | C |
| ANISOU | 3498 | CG | ASN | A | 631 | 2269 | 2093 | 2023 | 68 | −85 | 65 | A | C |
| ATOM | 3499 | OD1 | ASN | A | 631 | 19.985 | 2.593 | 7.216 | 1.00 | 16.14 | | A | O |
| ANISOU | 3499 | OD1 | ASN | A | 631 | 2243 | 1942 | 1945 | 23 | −77 | 176 | A | O |
| ATOM | 3500 | ND2 | ASN | A | 631 | 18.830 | 4.050 | 5.900 | 1.00 | 18.61 | | A | N |
| ANISOU | 3500 | ND2 | ASN | A | 631 | 2402 | 2304 | 2362 | 141 | −75 | −12 | A | N |
| ATOM | 3503 | C | ASN | A | 631 | 21.653 | 0.574 | 3.941 | 1.00 | 15.25 | | A | C |
| ANISOU | 3503 | C | ASN | A | 631 | 1952 | 1921 | 1919 | 9 | −14 | 43 | A | C |
| ATOM | 3504 | O | ASN | A | 631 | 21.066 | −0.496 | 3.911 | 1.00 | 14.96 | | A | O |
| ANISOU | 3504 | O | ASN | A | 631 | 2104 | 1781 | 1796 | −12 | −27 | 63 | A | O |
| ATOM | 3505 | N | LYS | A | 632 | 22.393 | 1.018 | 2.931 | 1.00 | 15.71 | | A | N |
| ANISOU | 3505 | N | LYS | A | 632 | 2046 | 1977 | 1945 | −22 | −23 | 79 | A | N |
| ATOM | 3507 | CA | LYS | A | 632 | 22.511 | 0.279 | 1.674 | 1.00 | 16.46 | | A | C |
| ANISOU | 3507 | CA | LYS | A | 632 | 2123 | 2082 | 2048 | −14 | −5 | 15 | A | C |
| ATOM | 3509 | CB | LYS | A | 632 | 23.235 | 1.134 | 0.605 | 1.00 | 17.14 | | A | C |
| ANISOU | 3509 | CB | LYS | A | 632 | 2275 | 2140 | 2096 | −11 | −14 | 60 | A | C |
| ATOM | 3512 | CG | LYS | A | 632 | 24.720 | 1.347 | 0.845 | 1.00 | 19.38 | | A | C |
| ANISOU | 3512 | CG | LYS | A | 632 | 2408 | 2456 | 2498 | −32 | −13 | 48 | A | C |
| ATOM | 3515 | CD | LYS | A | 632 | 25.458 | 1.946 | −0.371 | 1.00 | 22.31 | | A | C |
| ANISOU | 3515 | CD | LYS | A | 632 | 2923 | 2801 | 2751 | −20 | 62 | 44 | A | C |
| ATOM | 3518 | CE | LYS | A | 632 | 26.963 | 2.027 | −0.095 | 1.00 | 24.58 | | A | C |
| ANISOU | 3518 | CE | LYS | A | 632 | 3082 | 3122 | 3135 | −35 | −23 | 27 | A | C |
| ATOM | 3521 | NZ | LYS | A | 632 | 27.705 | 2.894 | −1.061 | 1.00 | 26.47 | | A | N |
| ANISOU | 3521 | NZ | LYS | A | 632 | 3387 | 3352 | 3317 | −17 | 57 | 30 | A | N |
| ATOM | 3525 | C | LYS | A | 632 | 23.194 | −1.074 | 1.833 | 1.00 | 15.77 | | A | C |
| ANISOU | 3525 | C | LYS | A | 632 | 2016 | 2031 | 1942 | −32 | −13 | 24 | A | C |
| ATOM | 3526 | O | LYS | A | 632 | 23.051 | −1.953 | 0.996 | 1.00 | 16.37 | | A | O |
| ANISOU | 3526 | O | LYS | A | 632 | 2199 | 2170 | 1850 | −15 | −30 | 7 | A | O |
| ATOM | 3527 | N | ASP | A | 633 | 23.937 | −1.253 | 2.919 | 1.00 | 15.01 | | A | N |
| ANISOU | 3527 | N | ASP | A | 633 | 1921 | 1944 | 1836 | −26 | −3 | −10 | A | N |
| ATOM | 3529 | CA | ASP | A | 633 | 24.661 | −2.493 | 3.139 | 1.00 | 15.01 | | A | C |
| ANISOU | 3529 | CA | ASP | A | 633 | 1895 | 1914 | 1893 | −22 | 0 | 8 | A | C |
| ATOM | 3531 | CB | ASP | A | 633 | 25.958 | −2.190 | 3.882 | 1.00 | 16.28 | | A | C |
| ANISOU | 3531 | CB | ASP | A | 633 | 2051 | 2039 | 2095 | 19 | −82 | −21 | A | C |
| ATOM | 3534 | CG | ASP | A | 633 | 27.019 | −1.561 | 2.966 | 1.00 | 19.59 | | A | C |
| ANISOU | 3534 | CG | ASP | A | 633 | 2490 | 2424 | 2529 | −40 | 98 | 35 | A | C |
| ATOM | 3535 | OD1 | ASP | A | 633 | 26.772 | −1.374 | 1.760 | 1.00 | 24.84 | | A | O |
| ANISOU | 3535 | OD1 | ASP | A | 633 | 3235 | 3123 | 3080 | −1 | −125 | 185 | A | O |
| ATOM | 3536 | OD2 | ASP | A | 633 | 28.154 | −1.266 | 3.350 | 1.00 | 24.27 | | A | O |
| ANISOU | 3536 | OD2 | ASP | A | 633 | 3011 | 3068 | 3140 | −140 | −93 | −75 | A | O |
| ATOM | 3537 | C | ASP | A | 633 | 23.869 | −3.555 | 3.896 | 1.00 | 13.27 | | A | C |
| ANISOU | 3537 | C | ASP | A | 633 | 1660 | 1734 | 1647 | 14 | −30 | −24 | A | C |
| ATOM | 3538 | O | ASP | A | 633 | 24.270 | −4.711 | 3.936 | 1.00 | 13.66 | | A | O |
| ANISOU | 3538 | O | ASP | A | 633 | 1712 | 1795 | 1680 | 2 | 7 | −23 | A | O |
| ATOM | 3539 | N | VAL | A | 634 | 22.737 | −3.157 | 4.479 | 1.00 | 11.49 | | A | N |
| ANISOU | 3539 | N | VAL | A | 634 | 1423 | 1491 | 1453 | −26 | −28 | 5 | A | N |
| ATOM | 3541 | CA | VAL | A | 634 | 22.008 | −4.016 | 5.386 | 1.00 | 11.05 | | A | C |
| ANISOU | 3541 | CA | VAL | A | 634 | 1405 | 1454 | 1337 | −5 | −35 | 15 | A | C |
| ATOM | 3543 | CB | VAL | A | 634 | 20.863 | −3.256 | 6.056 | 1.00 | 10.53 | | A | C |
| ANISOU | 3543 | CB | VAL | A | 634 | 1351 | 1365 | 1282 | −33 | −39 | 64 | A | C |
| ATOM | 3545 | CG1 | VAL | A | 634 | 19.950 | −4.217 | 6.852 | 1.00 | 11.29 | | A | C |
| ANISOU | 3545 | CG1 | VAL | A | 634 | 1363 | 1582 | 1344 | −27 | 121 | −4 | A | C |
| ATOM | 3549 | CG2 | VAL | A | 634 | 21.388 | −2.174 | 6.941 | 1.00 | 11.26 | | A | C |
| ANISOU | 3549 | CG2 | VAL | A | 634 | 1398 | 1428 | 1451 | −153 | 96 | 81 | A | C |
| ATOM | 3553 | C | VAL | A | 634 | 21.481 | −5.264 | 4.685 | 1.00 | 11.12 | | A | C |
| ANISOU | 3553 | C | VAL | A | 634 | 1404 | 1453 | 1366 | −12 | 0 | 35 | A | C |
| ATOM | 3554 | O | VAL | A | 634 | 21.681 | −6.371 | 5.174 | 1.00 | 10.89 | | A | O |
| ANISOU | 3554 | O | VAL | A | 634 | 1296 | 1492 | 1349 | 105 | 72 | −22 | A | O |
| ATOM | 3555 | N | ILE | A | 635 | 20.816 | −5.099 | 3.542 | 1.00 | 11.18 | | A | N |
| ANISOU | 3555 | N | ILE | A | 635 | 1387 | 1435 | 1424 | −49 | 3 | 64 | A | N |
| ATOM | 3557 | CA | ILE | A | 635 | 20.221 | −6.252 | 2.891 | 1.00 | 11.30 | | A | C |
| ANISOU | 3557 | CA | ILE | A | 635 | 1393 | 1486 | 1414 | −12 | −41 | 17 | A | C |
| ATOM | 3559 | CB | ILE | A | 635 | 19.316 | −5.847 | 1.719 | 1.00 | 11.18 | | A | C |
| ANISOU | 3559 | CB | ILE | A | 635 | 1329 | 1472 | 1444 | 22 | −10 | −5 | A | C |
| ATOM | 3561 | CG1 | ILE | A | 635 | 18.478 | −7.053 | 1.270 | 1.00 | 11.96 | | A | C |
| ANISOU | 3561 | CG1 | ILE | A | 635 | 1556 | 1507 | 1479 | −27 | −47 | −50 | A | C |
| ATOM | 3564 | CD1 | ILE | A | 635 | 17.492 | −7.555 | 2.279 | 1.00 | 12.52 | | A | C |
| ANISOU | 3564 | CD1 | ILE | A | 635 | 1484 | 1578 | 1694 | 60 | 9 | 4 | A | C |
| ATOM | 3568 | CG2 | ILE | A | 635 | 20.115 | −5.305 | 0.563 | 1.00 | 11.86 | | A | C |
| ANISOU | 3568 | CG2 | ILE | A | 635 | 1484 | 1608 | 1413 | −28 | −68 | 47 | A | C |
| ATOM | 3572 | C | ILE | A | 635 | 21.259 | −7.287 | 2.498 | 1.00 | 11.28 | | A | C |
| ANISOU | 3572 | C | ILE | A | 635 | 1409 | 1464 | 1412 | −25 | −34 | 24 | A | C |
| ATOM | 3573 | O | ILE | A | 635 | 21.030 | −8.481 | 2.660 | 1.00 | 11.42 | | A | O |
| ANISOU | 3573 | O | ILE | A | 635 | 1400 | 1440 | 1496 | −7 | −159 | −55 | A | O |
| ATOM | 3574 | N | GLY | A | 636 | 22.426 | −6.844 | 2.046 | 1.00 | 11.85 | | A | N |
| ANISOU | 3574 | N | GLY | A | 636 | 1458 | 1484 | 1560 | −12 | −25 | 23 | A | N |
| ATOM | 3576 | CA | GLY | A | 636 | 23.473 | −7.790 | 1.712 | 1.00 | 12.18 | | A | C |
| ANISOU | 3576 | CA | GLY | A | 636 | 1507 | 1563 | 1556 | −13 | 47 | 8 | A | C |
| ATOM | 3579 | C | GLY | A | 636 | 23.909 | −8.641 | 2.891 | 1.00 | 12.11 | | A | C |
| ANISOU | 3579 | C | GLY | A | 636 | 1509 | 1531 | 1561 | −1 | 38 | 0 | A | C |
| ATOM | 3580 | O | GLY | A | 636 | 24.137 | −9.842 | 2.762 | 1.00 | 12.65 | | A | O |
| ANISOU | 3580 | O | GLY | A | 636 | 1609 | 1535 | 1661 | 33 | 39 | −14 | A | O |
| ATOM | 3581 | N | VAL | A | 637 | 24.021 | −8.013 | 4.054 | 1.00 | 11.63 | | A | N |
| ANISOU | 3581 | N | VAL | A | 637 | 1400 | 1458 | 1560 | 39 | 9 | −25 | A | N |
| ATOM | 3583 | CA | VAL | A | 637 | 24.420 | −8.698 | 5.273 | 1.00 | 11.85 | | A | C |
| ANISOU | 3583 | CA | VAL | A | 637 | 1469 | 1539 | 1494 | 20 | 59 | −35 | A | C |
| ATOM | 3585 | CB | VAL | A | 637 | 24.685 | −7.642 | 6.391 | 1.00 | 12.84 | | A | C |
| ANISOU | 3585 | CB | VAL | A | 637 | 1581 | 1614 | 1682 | 10 | −46 | −47 | A | C |
| ATOM | 3587 | CG1 | VAL | A | 637 | 24.600 | −8.211 | 7.750 | 1.00 | 15.93 | | A | C |
| ANISOU | 3587 | CG1 | VAL | A | 637 | 2107 | 2036 | 1909 | −28 | −36 | −64 | A | C |
| ATOM | 3591 | CG2 | VAL | A | 637 | 26.022 | −6.963 | 6.152 | 1.00 | 14.08 | | A | C |
| ANISOU | 3591 | CG2 | VAL | A | 637 | 1825 | 1719 | 1805 | −80 | 21 | −75 | A | C |
| ATOM | 3595 | C | VAL | A | 637 | 23.338 | −9.723 | 5.662 | 1.00 | 10.63 | | A | C |
| ANISOU | 3595 | C | VAL | A | 637 | 1337 | 1366 | 1333 | 20 | 0 | −35 | A | C |
| ATOM | 3596 | O | VAL | A | 637 | 23.611 | −10.862 | 5.976 | 1.00 | 11.17 | | A | O |
| ANISOU | 3596 | O | VAL | A | 637 | 1276 | 1517 | 1451 | 100 | 18 | −115 | A | O |
| ATOM | 3597 | N | LEU | A | 638 | 22.079 | −9.325 | 5.619 | 1.00 | 10.88 | | A | N |
| ANISOU | 3597 | N | LEU | A | 638 | 1352 | 1350 | 1431 | 35 | 24 | −43 | A | N |
| ATOM | 3599 | CA | LEU | A | 638 | 21.002 | −10.244 | 5.992 | 1.00 | 11.05 | | A | C |
| ANISOU | 3599 | CA | LEU | A | 638 | 1461 | 1375 | 1361 | −15 | 40 | −26 | A | C |
| ATOM | 3601 | CB | LEU | A | 638 | 19.658 | −9.521 | 6.027 | 1.00 | 10.68 | | A | C |
| ANISOU | 3601 | CB | LEU | A | 638 | 1429 | 1309 | 1320 | −3 | 35 | −22 | A | C |
| ATOM | 3604 | CG | LEU | A | 638 | 19.547 | −8.344 | 7.011 | 1.00 | 10.27 | | A | C |
| ANISOU | 3604 | CG | LEU | A | 638 | 1344 | 1282 | 1277 | −76 | −1 | −7 | A | C |
| ATOM | 3606 | CD1 | LEU | A | 638 | 18.238 | −7.630 | 6.794 | 1.00 | 9.53 | | A | C |
| ANISOU | 3606 | CD1 | LEU | A | 638 | 1420 | 1197 | 1003 | −27 | 87 | −76 | A | C |
| ATOM | 3610 | CD2 | LEU | A | 638 | 19.732 | −8.792 | 8.440 | 1.00 | 10.25 | | A | C |
| ANISOU | 3610 | CD2 | LEU | A | 638 | 1304 | 1365 | 1226 | −26 | −11 | −46 | A | C |
| ATOM | 3614 | C | LEU | A | 638 | 20.915 | −11.413 | 5.013 | 1.00 | 11.41 | | A | C |
| ANISOU | 3614 | C | LEU | A | 638 | 1477 | 1379 | 1478 | 10 | 57 | −50 | A | C |
| ATOM | 3615 | O | LEU | A | 638 | 20.712 | −12.537 | 5.423 | 1.00 | 10.84 | | A | O |
| ANISOU | 3615 | O | LEU | A | 638 | 1420 | 1333 | 1364 | −17 | 123 | −103 | A | O |
| ATOM | 3616 | N | GLU | A | 639 | 21.083 | −11.132 | 3.727 | 1.00 | 12.44 | | A | N |
| ANISOU | 3616 | N | GLU | A | 639 | 1656 | 1525 | 1545 | −42 | 27 | −63 | A | N |
| ATOM | 3618 | CA | GLU | A | 639 | 21.074 | −12.190 | 2.705 | 1.00 | 13.00 | | A | C |
| ANISOU | 3618 | CA | GLU | A | 639 | 1690 | 1632 | 1615 | −14 | 32 | −62 | A | C |
| ATOM | 3620 | CB | GLU | A | 639 | 21.095 | −11.605 | 1.273 | 1.00 | 14.26 | | A | C |
| ANISOU | 3620 | CB | GLU | A | 639 | 1957 | 1762 | 1696 | −36 | 34 | −66 | A | C |
| ATOM | 3623 | CG | GLU | A | 639 | 19.797 | −10.934 | 0.839 | 1.00 | 17.67 | | A | C |
| ANISOU | 3623 | CG | GLU | A | 639 | 2268 | 2152 | 2291 | 24 | −11 | −51 | A | C |
| ATOM | 3626 | CD | GLU | A | 639 | 19.890 | −10.273 | −0.542 | 1.00 | 22.72 | | A | C |
| ANISOU | 3626 | CD | GLU | A | 639 | 3100 | 2823 | 2708 | 21 | 0 | 24 | A | C |
| ATOM | 3627 | OE1 | GLU | A | 639 | 21.009 | −10.130 | −1.081 | 1.00 | 26.70 | | A | O |
| ANISOU | 3627 | OE1 | GLU | A | 639 | 3484 | 3423 | 3237 | −81 | 115 | 45 | A | O |
| ATOM | 3628 | OE2 | GLU | A | 639 | 18.840 | −9.871 | −1.079 | 1.00 | 26.87 | | A | O |
| ANISOU | 3628 | OE2 | GLU | A | 639 | 3480 | 3331 | 3398 | 42 | −197 | −6 | A | O |
| ATOM | 3629 | C | GLU | A | 639 | 22.216 | −13.185 | 2.917 | 1.00 | 13.08 | | A | C |
| ANISOU | 3629 | C | GLU | A | 639 | 1741 | 1634 | 1594 | −12 | −4 | −48 | A | C |
| ATOM | 3630 | O | GLU | A | 639 | 22.049 | −14.344 | 2.645 | 1.00 | 13.54 | | A | O |
| ANISOU | 3630 | O | GLU | A | 639 | 1800 | 1633 | 1712 | 1 | −38 | −82 | A | O |
| ATOM | 3631 | N | LYS | A | 640 | 23.359 | −12.742 | 3.442 | 1.00 | 12.74 | | A | N |
| ANISOU | 3631 | N | LYS | A | 640 | 1633 | 1625 | 1583 | 1 | 16 | −74 | A | N |
| ATOM | 3633 | CA | LYS | A | 640 | 24.478 | −13.640 | 3.735 | 1.00 | 13.77 | | A | C |
| ANISOU | 3633 | CA | LYS | A | 640 | 1810 | 1698 | 1724 | 15 | 29 | −57 | A | C |
| ATOM | 3635 | CB | LYS | A | 640 | 25.762 | −12.841 | 3.937 | 1.00 | 14.76 | | A | C |
| ANISOU | 3635 | CB | LYS | A | 640 | 1814 | 1870 | 1922 | 79 | −19 | −39 | A | C |
| ATOM | 3638 | CG | LYS | A | 640 | 26.269 | −12.150 | 2.705 | 1.00 | 18.43 | | A | C |
| ANISOU | 3638 | CG | LYS | A | 640 | 2373 | 2339 | 2290 | 30 | 24 | 47 | A | C |
| ATOM | 3641 | CD | LYS | A | 640 | 27.669 | −11.557 | 2.921 | 1.00 | 22.16 | | A | C |
| ANISOU | 3641 | CD | LYS | A | 640 | 2652 | 2838 | 2927 | −72 | −22 | 25 | A | C |
| ATOM | 3644 | CE | LYS | A | 640 | 27.703 | −10.410 | 3.923 | 1.00 | 24.61 | | A | C |
| ANISOU | 3644 | CE | LYS | A | 640 | 3123 | 3082 | 3146 | −3 | −31 | −54 | A | C |
| ATOM | 3647 | NZ | LYS | A | 640 | 27.280 | −9.091 | 3.342 | 1.00 | 26.76 | | A | N |
| ANISOU | 3647 | NZ | LYS | A | 640 | 3415 | 3270 | 3481 | 22 | −90 | 42 | A | N |
| ATOM | 3651 | C | LYS | A | 640 | 24.222 | −14.495 | 4.969 | 1.00 | 13.21 | | A | C |
| ANISOU | 3651 | C | LYS | A | 640 | 1750 | 1627 | 1642 | 55 | −17 | −59 | A | C |
| ATOM | 3652 | O | LYS | A | 640 | 25.003 | −15.379 | 5.284 | 1.00 | 14.21 | | A | O |
| ANISOU | 3652 | O | LYS | A | 640 | 1849 | 1720 | 1830 | 113 | 74 | −123 | A | O |
| ATOM | 3653 | N | GLY | A | 641 | 23.165 | −14.174 | 5.710 | 1.00 | 12.64 | | A | N |
| ANISOU | 3653 | N | GLY | A | 641 | 1659 | 1512 | 1631 | 61 | 25 | −4 | A | N |
| ATOM | 3655 | CA | GLY | A | 641 | 22.779 | −14.925 | 6.891 | 1.00 | 12.25 | | A | C |
| ANISOU | 3655 | CA | GLY | A | 641 | 1573 | 1501 | 1581 | 78 | 62 | −74 | A | C |
| ATOM | 3658 | C | GLY | A | 641 | 23.279 | −14.317 | 8.179 | 1.00 | 12.26 | | A | C |
| ANISOU | 3658 | C | GLY | A | 641 | 1571 | 1525 | 1560 | 69 | 52 | −26 | A | C |
| ATOM | 3659 | O | GLY | A | 641 | 23.066 | −14.893 | 9.246 | 1.00 | 13.24 | | A | O |
| ANISOU | 3659 | O | GLY | A | 641 | 1826 | 1595 | 1610 | 105 | 87 | −74 | A | O |
| ATOM | 3660 | N | ASP | A | 642 | 23.948 | −13.170 | 8.085 | 1.00 | 11.89 | | A | N |
| ANISOU | 3660 | N | ASP | A | 642 | 1482 | 1543 | 1492 | 77 | 61 | −95 | A | N |
| ATOM | 3662 | CA | ASP | A | 642 | 24.444 | −12.472 | 9.264 | 1.00 | 11.79 | | A | C |
| ANISOU | 3662 | CA | ASP | A | 642 | 1506 | 1482 | 1490 | 57 | 16 | −51 | A | C |
| ATOM | 3664 | CB | ASP | A | 642 | 25.450 | −11.384 | 8.862 | 1.00 | 13.17 | | A | C |
| ANISOU | 3664 | CB | ASP | A | 642 | 1673 | 1652 | 1677 | 5 | −3 | −34 | A | C |
| ATOM | 3667 | CG | ASP | A | 642 | 26.755 | −11.922 | 8.340 | 1.00 | 16.93 | | A | C |
| ANISOU | 3667 | CG | ASP | A | 642 | 2055 | 2221 | 2153 | 50 | 20 | −29 | A | C |
| ATOM | 3668 | OD1 | ASP | A | 642 | 27.067 | −13.113 | 8.550 | 1.00 | 19.65 | | A | O |
| ANISOU | 3668 | OD1 | ASP | A | 642 | 2418 | 2245 | 2802 | 109 | 13 | −157 | A | O |
| ATOM | 3669 | OD2 | ASP | A | 642 | 27.559 | −11.174 | 7.734 | 1.00 | 21.95 | | A | O |
| ANISOU | 3669 | OD2 | ASP | A | 642 | 2641 | 2883 | 2816 | −148 | 157 | 28 | A | O |
| ATOM | 3670 | C | ASP | A | 642 | 23.254 | −11.811 | 9.997 | 1.00 | 10.88 | | A | C |
| ANISOU | 3670 | C | ASP | A | 642 | 1379 | 1386 | 1368 | 36 | −19 | −54 | A | C |
| ATOM | 3671 | O | ASP | A | 642 | 22.269 | −11.384 | 9.384 | 1.00 | 9.89 | | A | O |
| ANISOU | 3671 | O | ASP | A | 642 | 1325 | 1187 | 1245 | 48 | 91 | −99 | A | O |
| ATOM | 3672 | N | ARG | A | 643 | 23.394 | −11.705 | 11.309 | 1.00 | 10.17 | | A | N |
| ANISOU | 3672 | N | ARG | A | 643 | 1299 | 1312 | 1253 | 40 | 49 | 32 | A | N |
| ATOM | 3674 | CA | ARG | A | 643 | 22.358 | −11.145 | 12.170 | 1.00 | 10.74 | | A | C |
| ANISOU | 3674 | CA | ARG | A | 643 | 1403 | 1320 | 1358 | 39 | 12 | −7 | A | C |
| ATOM | 3676 | CB | ARG | A | 643 | 21.507 | −12.257 | 12.804 | 1.00 | 11.04 | | A | C |
| ANISOU | 3676 | CB | ARG | A | 643 | 1392 | 1433 | 1367 | 32 | 17 | 19 | A | C |
| ATOM | 3679 | CG | ARG | A | 643 | 20.724 | −13.122 | 11.814 | 1.00 | 11.65 | | A | C |
| ANISOU | 3679 | CG | ARG | A | 643 | 1613 | 1338 | 1475 | 24 | 66 | −11 | A | C |
| ATOM | 3682 | CD | ARG | A | 643 | 19.638 | −12.359 | 11.087 | 1.00 | 11.09 | | A | C |
| ANISOU | 3682 | CD | ARG | A | 643 | 1367 | 1546 | 1298 | −35 | 75 | −80 | A | C |
| ATOM | 3685 | NE | ARG | A | 643 | 18.829 | −13.196 | 10.204 | 1.00 | 12.34 | | A | N |
| ANISOU | 3685 | NE | ARG | A | 643 | 1455 | 1520 | 1714 | −103 | 55 | −32 | A | N |
| ATOM | 3687 | CZ | ARG | A | 643 | 19.073 | −13.441 | 8.918 | 1.00 | 12.04 | | A | C |
| ANISOU | 3687 | CZ | ARG | A | 643 | 1477 | 1458 | 1639 | −51 | 78 | 5 | A | C |
| ATOM | 3688 | NH1 | ARG | A | 643 | 20.134 | −12.927 | 8.306 | 1.00 | 8.18 | | A | N |
| ANISOU | 3688 | NH1 | ARG | A | 643 | 1163 | 1026 | 917 | 10 | 72 | −183 | A | N |
| ATOM | 3691 | NH2 | ARG | A | 643 | 18.234 | −14.212 | 8.225 | 1.00 | 12.82 | | A | N |
| ANISOU | 3691 | NH2 | ARG | A | 643 | 1696 | 1600 | 1572 | 59 | 58 | −141 | A | N |
| ATOM | 3694 | C | ARG | A | 643 | 22.990 | −10.277 | 13.248 | 1.00 | 10.07 | | A | C |
| ANISOU | 3694 | C | ARG | A | 643 | 1304 | 1257 | 1264 | 11 | 32 | 23 | A | C |
| ATOM | 3695 | O | ARG | A | 643 | 24.202 | −10.312 | 13.480 | 1.00 | 11.11 | | A | O |
| ANISOU | 3695 | O | ARG | A | 643 | 1409 | 1440 | 1370 | 50 | −37 | −52 | A | O |
| ATOM | 3696 | N | LEU | A | 644 | 22.171 | −9.452 | 13.881 | 1.00 | 9.23 | | A | N |
| ANISOU | 3696 | N | LEU | A | 644 | 1088 | 1167 | 1250 | 11 | 0 | 12 | A | N |
| ATOM | 3698 | CA | LEU | A | 644 | 22.605 | −8.720 | 15.057 | 1.00 | 8.97 | | A | C |
| ANISOU | 3698 | CA | LEU | A | 644 | 1120 | 1110 | 1178 | −26 | 41 | 30 | A | C |
| ATOM | 3700 | CB | LEU | A | 644 | 21.442 | −7.941 | 15.659 | 1.00 | 8.90 | | A | C |
| ANISOU | 3700 | CB | LEU | A | 644 | 1134 | 1133 | 1111 | 16 | −35 | −40 | A | C |
| ATOM | 3703 | CG | LEU | A | 644 | 20.872 | −6.812 | 14.792 | 1.00 | 8.68 | | A | C |
| ANISOU | 3703 | CG | LEU | A | 644 | 1160 | 967 | 1168 | −50 | 24 | 5 | A | C |
| ATOM | 3705 | CD1 | LEU | A | 644 | 19.472 | −6.410 | 15.278 | 1.00 | 9.88 | | A | C |
| ANISOU | 3705 | CD1 | LEU | A | 644 | 1287 | 1162 | 1305 | −33 | −26 | 4 | A | C |
| ATOM | 3709 | CD2 | LEU | A | 644 | 21.782 | −5.605 | 14.760 | 1.00 | 9.21 | | A | C |
| ANISOU | 3709 | CD2 | LEU | A | 644 | 1135 | 1211 | 1152 | −138 | −38 | 80 | A | C |
| ATOM | 3713 | C | LEU | A | 644 | 23.176 | −9.701 | 16.080 | 1.00 | 8.86 | | A | C |
| ANISOU | 3713 | C | LEU | A | 644 | 1091 | 1091 | 1183 | −28 | 32 | 36 | A | C |
| ATOM | 3714 | O | LEU | A | 644 | 22.570 | −10.754 | 16.337 | 1.00 | 9.64 | | A | O |
| ANISOU | 3714 | O | LEU | A | 644 | 1216 | 1033 | 1413 | −109 | 4 | 33 | A | O |
| ATOM | 3715 | N | PRO | A | 645 | 24.316 | −9.380 | 16.685 | 1.00 | 9.03 | | A | N |
| ANISOU | 3715 | N | PRO | A | 645 | 1145 | 1108 | 1174 | −66 | 26 | 19 | A | N |
| ATOM | 3716 | CA | PRO | A | 645 | 24.919 | −10.294 | 17.665 | 1.00 | 8.83 | | A | C |
| ANISOU | 3716 | CA | PRO | A | 645 | 1092 | 1090 | 1172 | −9 | 2 | −12 | A | C |
| ATOM | 3718 | CB | PRO | A | 645 | 26.331 | −9.729 | 17.832 | 1.00 | 10.39 | | A | C |
| ANISOU | 3718 | CB | PRO | A | 645 | 1214 | 1345 | 1387 | −91 | −17 | 34 | A | C |
| ATOM | 3721 | CG | PRO | A | 645 | 26.141 | −8.252 | 17.675 | 1.00 | 11.21 | | A | C |
| ANISOU | 3721 | CG | PRO | A | 645 | 1375 | 1399 | 1483 | −173 | −50 | 23 | A | C |
| ATOM | 3724 | CD | PRO | A | 645 | 25.104 | −8.133 | 16.539 | 1.00 | 10.07 | | A | C |
| ANISOU | 3724 | CD | PRO | A | 645 | 1273 | 1288 | 1263 | −88 | 14 | 133 | A | C |
| ATOM | 3727 | C | PRO | A | 645 | 24.188 | −10.276 | 18.993 | 1.00 | 8.79 | | A | C |
| ANISOU | 3727 | C | PRO | A | 645 | 1117 | 1066 | 1155 | 10 | 29 | 33 | A | C |
| ATOM | 3728 | O | PRO | A | 645 | 23.520 | −9.313 | 19.322 | 1.00 | 9.49 | | A | O |
| ANISOU | 3728 | O | PRO | A | 645 | 1330 | 1031 | 1242 | 131 | 70 | 130 | A | O |
| ATOM | 3729 | N | LYS | A | 646 | 24.381 | −11.295 | 19.803 | 1.00 | 8.65 | | A | N |
| ANISOU | 3729 | N | LYS | A | 646 | 1172 | 1019 | 1096 | 14 | 48 | 25 | A | N |
| ATOM | 3731 | CA | LYS | A | 646 | 23.756 | −11.323 | 21.111 | 1.00 | 9.22 | | A | C |
| ANISOU | 3731 | CA | LYS | A | 646 | 1217 | 1096 | 1189 | −5 | 49 | 15 | A | C |
| ATOM | 3733 | CB | LYS | A | 646 | 24.026 | −12.666 | 21.801 | 1.00 | 10.45 | | A | C |
| ANISOU | 3733 | CB | LYS | A | 646 | 1475 | 1215 | 1280 | −32 | −24 | 27 | A | C |
| ATOM | 3736 | CG | LYS | A | 646 | 23.202 | −12.745 | 23.076 | 1.00 | 12.24 | | A | C |
| ANISOU | 3736 | CG | LYS | A | 646 | 1631 | 1586 | 1431 | −97 | 89 | −12 | A | C |
| ATOM | 3739 | CD | LYS | A | 646 | 23.314 | −14.080 | 23.805 | 1.00 | 15.66 | | A | C |
| ANISOU | 3739 | CD | LYS | A | 646 | 2158 | 1847 | 1945 | −24 | −7 | 72 | A | C |
| ATOM | 3742 | CE | LYS | A | 646 | 24.596 | −14.159 | 24.612 | 1.00 | 16.74 | | A | C |
| ANISOU | 3742 | CE | LYS | A | 646 | 2145 | 2027 | 2189 | −7 | −35 | 75 | A | C |
| ATOM | 3745 | NZ | LYS | A | 646 | 24.740 | −13.087 | 25.672 | 1.00 | 16.74 | | A | N |
| ANISOU | 3745 | NZ | LYS | A | 646 | 2122 | 2081 | 2158 | 13 | −57 | 29 | A | N |
| ATOM | 3749 | C | LYS | A | 646 | 24.268 | −10.174 | 21.975 | 1.00 | 8.79 | | A | C |
| ANISOU | 3749 | C | LYS | A | 646 | 1117 | 1147 | 1072 | −37 | 65 | 17 | A | C |
| ATOM | 3750 | O | LYS | A | 646 | 25.483 | −10.056 | 22.190 | 1.00 | 9.36 | | A | O |
| ANISOU | 3750 | O | LYS | A | 646 | 1116 | 1274 | 1166 | −99 | 16 | −103 | A | O |
| ATOM | 3751 | N | PRO | A | 647 | 23.380 | −9.325 | 22.495 | 1.00 | 9.12 | | A | N |
| ANISOU | 3751 | N | PRO | A | 647 | 1134 | 1137 | 1192 | −24 | 5 | 24 | A | N |
| ATOM | 3752 | CA | PRO | A | 647 | 23.816 | −8.335 | 23.492 | 1.00 | 9.77 | | A | C |
| ANISOU | 3752 | CA | PRO | A | 647 | 1255 | 1202 | 1252 | −27 | 45 | 9 | A | C |
| ATOM | 3754 | CB | PRO | A | 647 | 22.517 | −7.593 | 23.838 | 1.00 | 9.88 | | A | C |
| ANISOU | 3754 | CB | PRO | A | 647 | 1247 | 1242 | 1264 | −32 | 58 | 17 | A | C |
| ATOM | 3757 | CG | PRO | A | 647 | 21.672 | −7.760 | 22.648 | 1.00 | 10.13 | | A | C |
| ANISOU | 3757 | CG | PRO | A | 647 | 1322 | 1330 | 1195 | 12 | 92 | −62 | A | C |
| ATOM | 3760 | CD | PRO | A | 647 | 21.944 | −9.159 | 22.159 | 1.00 | 9.19 | | A | C |
| ANISOU | 3760 | CD | PRO | A | 647 | 1102 | 1182 | 1208 | −66 | 62 | −25 | A | C |
| ATOM | 3763 | C | PRO | A | 647 | 24.421 | −9.023 | 24.713 | 1.00 | 9.70 | | A | C |
| ANISOU | 3763 | C | PRO | A | 647 | 1264 | 1200 | 1221 | −18 | 57 | −31 | A | C |
| ATOM | 3764 | O | PRO | A | 647 | 23.980 | −10.129 | 25.069 | 1.00 | 9.56 | | A | O |
| ANISOU | 3764 | O | PRO | A | 647 | 1272 | 1232 | 1126 | 61 | 47 | 137 | A | O |
| ATOM | 3765 | N | ASP | A | 648 | 25.412 | −8.395 | 25.331 | 1.00 | 10.93 | | A | N |
| ANISOU | 3765 | N | ASP | A | 648 | 1409 | 1374 | 1367 | −15 | −47 | 30 | A | N |
| ATOM | 3767 | CA | ASP | A | 648 | 26.106 | −9.004 | 26.465 | 1.00 | 12.88 | | A | C |
| ANISOU | 3767 | CA | ASP | A | 648 | 1616 | 1657 | 1621 | −20 | −20 | 55 | A | C |
| ATOM | 3769 | CB | ASP | A | 648 | 27.097 | −8.015 | 27.064 | 1.00 | 14.05 | | A | C |
| ANISOU | 3769 | CB | ASP | A | 648 | 1815 | 1786 | 1737 | −47 | −27 | 51 | A | C |
| ATOM | 3772 | CG | ASP | A | 648 | 28.032 | −8.674 | 28.039 | 1.00 | 16.34 | | A | C |
| ANISOU | 3772 | CG | ASP | A | 648 | 1947 | 2173 | 2089 | −3 | −79 | 120 | A | C |
| ATOM | 3773 | OD1 | ASP | A | 648 | 28.724 | −9.649 | 27.667 | 1.00 | 18.87 | | A | O |
| ANISOU | 3773 | OD1 | ASP | A | 648 | 2317 | 2422 | 2429 | 23 | −40 | 85 | A | O |
| ATOM | 3774 | OD2 | ASP | A | 648 | 28.101 | −8.282 | 29.203 | 1.00 | 22.16 | | A | O |
| ANISOU | 3774 | OD2 | ASP | A | 648 | 2868 | 3064 | 2484 | −75 | −60 | −50 | A | O |
| ATOM | 3775 | C | ASP | A | 648 | 25.171 | −9.560 | 27.553 | 1.00 | 13.03 | | A | C |
| ANISOU | 3775 | C | ASP | A | 648 | 1705 | 1634 | 1611 | −6 | −8 | 50 | A | C |
| ATOM | 3776 | O | ASP | A | 648 | 25.364 | −10.693 | 28.018 | 1.00 | 13.83 | | A | O |
| ANISOU | 3776 | O | ASP | A | 648 | 1774 | 1758 | 1721 | 5 | −70 | 164 | A | O |
| ATOM | 3777 | N | LEU | A | 649 | 24.149 | −8.786 | 27.915 | 1.00 | 12.78 | | A | N |
| ANISOU | 3777 | N | LEU | A | 649 | 1646 | 1649 | 1561 | −39 | 12 | 53 | A | N |
| ATOM | 3779 | CA | LEU | A | 649 | 23.264 | −9.140 | 29.021 | 1.00 | 13.30 | | A | C |
| ANISOU | 3779 | CA | LEU | A | 649 | 1688 | 1718 | 1644 | −23 | 31 | 30 | A | C |
| ATOM | 3781 | CB | LEU | A | 649 | 22.863 | −7.892 | 29.802 | 1.00 | 14.42 | | A | C |
| ANISOU | 3781 | CB | LEU | A | 649 | 1918 | 1812 | 1746 | −27 | 91 | 48 | A | C |
| ATOM | 3784 | CG | LEU | A | 649 | 24.018 | −7.251 | 30.578 | 1.00 | 17.26 | | A | C |
| ANISOU | 3784 | CG | LEU | A | 649 | 2139 | 2165 | 2252 | −76 | −15 | 36 | A | C |
| ATOM | 3786 | CD1 | LEU | A | 649 | 23.487 | −6.172 | 31.477 | 1.00 | 18.90 | | A | C |
| ANISOU | 3786 | CD1 | LEU | A | 649 | 2457 | 2412 | 2310 | −10 | 64 | −22 | A | C |
| ATOM | 3790 | CD2 | LEU | A | 649 | 24.837 | −8.239 | 31.400 | 1.00 | 18.74 | | A | C |
| ANISOU | 3790 | CD2 | LEU | A | 649 | 2434 | 2340 | 2344 | −27 | −47 | 29 | A | C |
| ATOM | 3794 | C | LEU | A | 649 | 22.023 | −9.898 | 28.582 | 1.00 | 12.89 | | A | C |
| ANISOU | 3794 | C | LEU | A | 649 | 1674 | 1674 | 1549 | −40 | 53 | 11 | A | C |
| ATOM | 3795 | O | LEU | A | 649 | 21.196 | −10.263 | 29.410 | 1.00 | 14.26 | | A | O |
| ANISOU | 3795 | O | LEU | A | 649 | 1827 | 1867 | 1723 | −137 | 203 | −12 | A | O |
| ATOM | 3796 | N | CYS | A | 650 | 21.901 | −10.178 | 27.293 | 1.00 | 11.79 | | A | N |
| ANISOU | 3796 | N | CYS | A | 650 | 1537 | 1537 | 1405 | −36 | 72 | 38 | A | N |
| ATOM | 3798 | CA | CYS | A | 650 | 20.795 | −10.967 | 26.806 | 1.00 | 11.54 | | A | C |
| ANISOU | 3798 | CA | CYS | A | 650 | 1456 | 1487 | 1441 | 45 | 32 | 20 | A | C |
| ATOM | 3800 | CB | CYS | A | 650 | 20.692 | −10.790 | 25.289 | 1.00 | 11.36 | | A | C |
| ANISOU | 3800 | CB | CYS | A | 650 | 1450 | 1460 | 1405 | 68 | 18 | 0 | A | C |
| ATOM | 3803 | SG | CYS | A | 650 | 19.303 | −11.660 | 24.541 | 1.00 | 11.66 | | A | S |
| ANISOU | 3803 | SG | CYS | A | 650 | 1267 | 1459 | 1704 | −18 | 63 | 226 | A | S |
| ATOM | 3804 | C | CYS | A | 650 | 20.955 | −12.455 | 27.154 | 1.00 | 12.36 | | A | C |
| ANISOU | 3804 | C | CYS | A | 650 | 1592 | 1586 | 1518 | 62 | 27 | 57 | A | C |
| ATOM | 3805 | O | CYS | A | 650 | 21.955 | −13.058 | 26.782 | 1.00 | 12.76 | | A | O |
| ANISOU | 3805 | O | CYS | A | 650 | 1653 | 1586 | 1608 | 93 | 60 | 78 | A | O |
| ATOM | 3806 | N | PRO | A | 651 | 19.971 | −13.037 | 27.854 | 1.00 | 12.87 | | A | N |
| ANISOU | 3806 | N | PRO | A | 651 | 1684 | 1537 | 1669 | 12 | 13 | 82 | A | N |
| ATOM | 3807 | CA | PRO | A | 651 | 19.951 | −14.479 | 28.113 | 1.00 | 13.18 | | A | C |
| ANISOU | 3807 | CA | PRO | A | 651 | 1681 | 1611 | 1713 | −19 | −27 | 10 | A | C |
| ATOM | 3809 | CB | PRO | A | 651 | 18.565 | −14.681 | 28.747 | 1.00 | 13.38 | | A | C |
| ANISOU | 3809 | CB | PRO | A | 651 | 1800 | 1640 | 1643 | −86 | 60 | 25 | A | C |
| ATOM | 3812 | CG | PRO | A | 651 | 18.243 | −13.418 | 29.381 | 1.00 | 14.43 | | A | C |
| ANISOU | 3812 | CG | PRO | A | 651 | 1817 | 1781 | 1884 | −21 | 21 | 80 | A | C |
| ATOM | 3815 | CD | PRO | A | 651 | 18.812 | −12.370 | 28.476 | 1.00 | 13.39 | | A | C |
| ANISOU | 3815 | CD | PRO | A | 651 | 1734 | 1739 | 1611 | −46 | 38 | 90 | A | C |
| ATOM | 3818 | C | PRO | A | 651 | 20.038 | −15.273 | 26.797 | 1.00 | 12.61 | | A | C |
| ANISOU | 3818 | C | PRO | A | 651 | 1610 | 1521 | 1657 | −26 | −50 | 21 | A | C |
| ATOM | 3819 | O | PRO | A | 651 | 19.355 | −14.918 | 25.853 | 1.00 | 11.79 | | A | O |
| ANISOU | 3819 | O | PRO | A | 651 | 1532 | 1282 | 1664 | −5 | −135 | 223 | A | O |
| ATOM | 3820 | N | PRO | A | 652 | 20.867 | −16.303 | 26.703 | 1.00 | 13.43 | | A | N |
| ANISOU | 3820 | N | PRO | A | 652 | 1665 | 1683 | 1753 | 46 | −124 | 69 | A | N |
| ATOM | 3821 | CA | PRO | A | 652 | 20.896 | −17.142 | 25.503 | 1.00 | 13.03 | | A | C |
| ANISOU | 3821 | CA | PRO | A | 652 | 1648 | 1583 | 1718 | 24 | −79 | 79 | A | C |
| ATOM | 3823 | CB | PRO | A | 652 | 21.794 | −18.317 | 25.917 | 1.00 | 14.64 | | A | C |
| ANISOU | 3823 | CB | PRO | A | 652 | 1883 | 1729 | 1950 | 48 | −76 | 45 | A | C |
| ATOM | 3826 | CG | PRO | A | 652 | 22.676 | −17.726 | 26.935 | 1.00 | 13.89 | | A | C |
| ANISOU | 3826 | CG | PRO | A | 652 | 1659 | 1752 | 1866 | 84 | −100 | 111 | A | C |
| ATOM | 3829 | CD | PRO | A | 652 | 21.904 | −16.682 | 27.680 | 1.00 | 14.14 | | A | C |
| ANISOU | 3829 | CD | PRO | A | 652 | 1710 | 1804 | 1856 | 15 | −163 | 52 | A | C |
| ATOM | 3832 | C | PRO | A | 652 | 19.513 | −17.594 | 24.987 | 1.00 | 12.68 | | A | C |
| ANISOU | 3832 | C | PRO | A | 652 | 1619 | 1532 | 1667 | 26 | −4 | 52 | A | C |
| ATOM | 3833 | O | PRO | A | 652 | 19.282 | −17.552 | 23.788 | 1.00 | 11.79 | | A | O |
| ANISOU | 3833 | O | PRO | A | 652 | 1536 | 1290 | 1652 | 46 | −71 | 124 | A | O |
| ATOM | 3834 | N | VAL | A | 653 | 18.613 | −17.983 | 25.878 | 1.00 | 12.27 | | A | N |
| ANISOU | 3834 | N | VAL | A | 653 | 1597 | 1420 | 1644 | 9 | −14 | 62 | A | N |
| ATOM | 3836 | CA | VAL | A | 653 | 17.281 | −18.416 | 25.481 | 1.00 | 12.50 | | A | C |
| ANISOU | 3836 | CA | VAL | A | 653 | 1605 | 1486 | 1655 | 26 | −5 | 34 | A | C |
| ATOM | 3838 | CB | VAL | A | 653 | 16.469 | −18.936 | 26.708 | 1.00 | 13.38 | | A | C |
| ANISOU | 3838 | CB | VAL | A | 653 | 1764 | 1651 | 1667 | 29 | 16 | 69 | A | C |
| ATOM | 3840 | CG1 | VAL | A | 653 | 16.253 | −17.882 | 27.778 | 1.00 | 15.20 | | A | C |
| ANISOU | 3840 | CG1 | VAL | A | 653 | 2033 | 1871 | 1871 | −10 | −22 | −18 | A | C |
| ATOM | 3844 | CG2 | VAL | A | 653 | 15.128 | −19.484 | 26.305 | 1.00 | 14.80 | | A | C |
| ANISOU | 3844 | CG2 | VAL | A | 653 | 1877 | 1758 | 1985 | −6 | −46 | −2 | A | C |
| ATOM | 3848 | C | VAL | A | 653 | 16.529 | −17.293 | 24.764 | 1.00 | 11.55 | | A | C |
| ANISOU | 3848 | C | VAL | A | 653 | 1522 | 1370 | 1497 | −6 | 12 | 35 | A | C |
| ATOM | 3849 | O | VAL | A | 653 | 15.803 | −17.533 | 23.784 | 1.00 | 11.72 | | A | O |
| ANISOU | 3849 | O | VAL | A | 653 | 1487 | 1334 | 1631 | 25 | 37 | 28 | A | O |
| ATOM | 3850 | N | LEU | A | 654 | 16.725 | −16.061 | 25.234 | 1.00 | 10.59 | | A | N |
| ANISOU | 3850 | N | LEU | A | 654 | 1369 | 1240 | 1414 | 3 | −7 | 91 | A | N |
| ATOM | 3852 | CA | LEU | A | 654 | 16.111 | −14.926 | 24.553 | 1.00 | 10.31 | | A | C |
| ANISOU | 3852 | CA | LEU | A | 654 | 1279 | 1271 | 1366 | 29 | −58 | 80 | A | C |
| ATOM | 3854 | CB | LEU | A | 654 | 16.141 | −13.704 | 25.461 | 1.00 | 10.74 | | A | C |
| ANISOU | 3854 | CB | LEU | A | 654 | 1411 | 1293 | 1375 | 61 | 37 | 61 | A | C |
| ATOM | 3857 | CG | LEU | A | 654 | 15.488 | −12.434 | 24.897 | 1.00 | 10.90 | | A | C |
| ANISOU | 3857 | CG | LEU | A | 654 | 1365 | 1352 | 1424 | 80 | 28 | 54 | A | C |
| ATOM | 3859 | CD1 | LEU | A | 654 | 14.034 | −12.661 | 24.526 | 1.00 | 12.25 | | A | C |
| ANISOU | 3859 | CD1 | LEU | A | 654 | 1549 | 1513 | 1593 | −63 | 62 | 49 | A | C |
| ATOM | 3863 | CD2 | LEU | A | 654 | 15.658 | −11.283 | 25.870 | 1.00 | 13.59 | | A | C |
| ANISOU | 3863 | CD2 | LEU | A | 654 | 1829 | 1668 | 1667 | −25 | 74 | −61 | A | C |
| ATOM | 3867 | C | LEU | A | 654 | 16.748 | −14.626 | 23.194 | 1.00 | 9.80 | | A | C |
| ANISOU | 3867 | C | LEU | A | 654 | 1217 | 1122 | 1383 | 38 | −40 | 18 | A | C |
| ATOM | 3868 | O | LEU | A | 654 | 16.050 | −14.304 | 22.234 | 1.00 | 10.34 | | A | O |
| ANISOU | 3868 | O | LEU | A | 654 | 1264 | 1232 | 1432 | 20 | −54 | 53 | A | O |
| ATOM | 3869 | N | TYR | A | 655 | 18.064 | −14.745 | 23.086 | 1.00 | 9.87 | | A | N |
| ANISOU | 3869 | N | TYR | A | 655 | 1236 | 1163 | 1351 | 18 | 18 | 117 | A | N |
| ATOM | 3871 | CA | TYR | A | 655 | 18.691 | −14.605 | 21.780 | 1.00 | 9.35 | | A | C |
| ANISOU | 3871 | CA | TYR | A | 655 | 1195 | 1144 | 1210 | 12 | 0 | −29 | A | C |
| ATOM | 3873 | CB | TYR | A | 655 | 20.203 | −14.628 | 21.896 | 1.00 | 8.79 | | A | C |
| ANISOU | 3873 | CB | TYR | A | 655 | 1147 | 1073 | 1118 | 30 | 0 | 44 | A | C |
| ATOM | 3876 | CG | TYR | A | 655 | 20.876 | −14.257 | 20.615 | 1.00 | 9.30 | | A | C |
| ANISOU | 3876 | CG | TYR | A | 655 | 1057 | 1198 | 1277 | −17 | 60 | 125 | A | C |
| ATOM | 3877 | CD1 | TYR | A | 655 | 20.735 | −12.989 | 20.095 | 1.00 | 9.13 | | A | C |
| ANISOU | 3877 | CD1 | TYR | A | 655 | 997 | 1062 | 1409 | 51 | −33 | 65 | A | C |
| ATOM | 3879 | CE1 | TYR | A | 655 | 21.335 | −12.634 | 18.891 | 1.00 | 9.04 | | A | C |
| ANISOU | 3879 | CE1 | TYR | A | 655 | 1110 | 1023 | 1301 | −81 | −9 | −42 | A | C |
| ATOM | 3881 | CZ | TYR | A | 655 | 22.097 | −13.558 | 18.203 | 1.00 | 10.17 | | A | C |
| ANISOU | 3881 | CZ | TYR | A | 655 | 1228 | 1269 | 1366 | −32 | 85 | 21 | A | C |
| ATOM | 3882 | OH | TYR | A | 655 | 22.675 | −13.209 | 17.020 | 1.00 | 12.04 | | A | O |
| ANISOU | 3882 | OH | TYR | A | 655 | 1460 | 1598 | 1517 | −29 | 248 | −27 | A | O |
| ATOM | 3884 | CE2 | TYR | A | 655 | 22.238 | −14.845 | 18.695 | 1.00 | 10.02 | | A | C |
| ANISOU | 3884 | CE2 | TYR | A | 655 | 1312 | 1224 | 1271 | 95 | 102 | −12 | A | C |
| ATOM | 3886 | CD2 | TYR | A | 655 | 21.635 | −15.186 | 19.902 | 1.00 | 9.51 | | A | C |
| ANISOU | 3886 | CD2 | TYR | A | 655 | 1169 | 1128 | 1316 | 95 | 36 | 173 | A | C |
| ATOM | 3888 | C | TYR | A | 655 | 18.194 | −15.670 | 20.789 | 1.00 | 9.65 | | A | C |
| ANISOU | 3888 | C | TYR | A | 655 | 1216 | 1137 | 1313 | 37 | 0 | −15 | A | C |
| ATOM | 3889 | O | TYR | A | 655 | 17.978 | −15.375 | 19.643 | 1.00 | 10.05 | | A | O |
| ANISOU | 3889 | O | TYR | A | 655 | 1207 | 1195 | 1415 | 30 | 58 | −67 | A | O |
| ATOM | 3890 | N | THR | A | 656 | 17.986 | −16.896 | 21.245 | 1.00 | 10.25 | | A | N |
| ANISOU | 3890 | N | THR | A | 656 | 1328 | 1203 | 1360 | 11 | −7 | −43 | A | N |
| ATOM | 3892 | CA | THR | A | 656 | 17.441 | −17.911 | 20.338 | 1.00 | 11.61 | | A | C |
| ANISOU | 3892 | CA | THR | A | 656 | 1548 | 1345 | 1516 | −3 | −48 | −8 | A | C |
| ATOM | 3894 | CB | THR | A | 656 | 17.341 | −19.223 | 21.104 | 1.00 | 12.32 | | A | C |
| ANISOU | 3894 | CB | THR | A | 656 | 1614 | 1469 | 1597 | −40 | −43 | 39 | A | C |
| ATOM | 3896 | OG1 | THR | A | 656 | 18.672 | −19.666 | 21.427 | 1.00 | 14.62 | | A | O |
| ANISOU | 3896 | OG1 | THR | A | 656 | 1925 | 1540 | 2089 | 151 | −187 | 79 | A | O |
| ATOM | 3898 | CG2 | THR | A | 656 | 16.743 | −20.321 | 20.246 | 1.00 | 13.85 | | A | C |
| ANISOU | 3898 | CG2 | THR | A | 656 | 1924 | 1485 | 1852 | 17 | 13 | −75 | A | C |
| ATOM | 3902 | C | THR | A | 656 | 16.068 | −17.470 | 19.808 | 1.00 | 11.48 | | A | C |
| ANISOU | 3902 | C | THR | A | 656 | 1500 | 1366 | 1493 | −78 | 13 | 63 | A | C |
| ATOM | 3903 | O | THR | A | 656 | 15.760 | −17.625 | 18.625 | 1.00 | 12.21 | | A | O |
| ANISOU | 3903 | O | THR | A | 656 | 1610 | 1445 | 1582 | −17 | −80 | 64 | A | O |
| ATOM | 3904 | N | LEU | A | 657 | 15.256 | −16.904 | 20.678 | 1.00 | 11.37 | | A | N |
| ANISOU | 3904 | N | LEU | A | 657 | 1456 | 1359 | 1504 | −48 | −71 | 71 | A | N |
| ATOM | 3906 | CA | LEU | A | 657 | 13.967 | −16.388 | 20.270 | 1.00 | 11.26 | | A | C |
| ANISOU | 3906 | CA | LEU | A | 657 | 1370 | 1402 | 1504 | −10 | −47 | 46 | A | C |
| ATOM | 3908 | CB | LEU | A | 657 | 13.194 | −15.883 | 21.490 | 1.00 | 12.25 | | A | C |
| ANISOU | 3908 | CB | LEU | A | 657 | 1537 | 1510 | 1606 | 36 | −40 | −46 | A | C |
| ATOM | 3911 | CG | LEU | A | 657 | 11.691 | −15.721 | 21.318 | 1.00 | 15.01 | | A | C |
| ANISOU | 3911 | CG | LEU | A | 657 | 1807 | 1933 | 1962 | −50 | 14 | 68 | A | C |
| ATOM | 3913 | CD1 | LEU | A | 657 | 11.055 | −17.063 | 21.001 | 1.00 | 16.78 | | A | C |
| ANISOU | 3913 | CD1 | LEU | A | 657 | 2124 | 2040 | 2210 | −138 | 61 | −141 | A | C |
| ATOM | 3917 | CD2 | LEU | A | 657 | 11.099 | −15.144 | 22.592 | 1.00 | 16.43 | | A | C |
| ANISOU | 3917 | CD2 | LEU | A | 657 | 2124 | 1949 | 2167 | 48 | 52 | −153 | A | C |
| ATOM | 3921 | C | LEU | A | 657 | 14.107 | −15.291 | 19.228 | 1.00 | 10.50 | | A | C |
| ANISOU | 3921 | C | LEU | A | 657 | 1237 | 1328 | 1424 | −25 | −59 | 9 | A | C |
| ATOM | 3922 | O | LEU | A | 657 | 13.389 | −15.290 | 18.221 | 1.00 | 11.31 | | A | O |
| ANISOU | 3922 | O | LEU | A | 657 | 1323 | 1397 | 1575 | 140 | −163 | 13 | A | O |
| ATOM | 3923 | N | MET | A | 658 | 15.016 | −14.348 | 19.466 | 1.00 | 9.64 | | A | N |
| ANISOU | 3923 | N | MET | A | 658 | 1161 | 1248 | 1252 | 16 | −70 | 37 | A | N |
| ATOM | 3925 | CA | MET | A | 658 | 15.275 | −13.301 | 18.493 | 1.00 | 9.56 | | A | C |
| ANISOU | 3925 | CA | MET | A | 658 | 1159 | 1209 | 1264 | 51 | −40 | 67 | A | C |
| ATOM | 3927 | CB | MET | A | 658 | 16.419 | −12.415 | 18.942 | 1.00 | 9.94 | | A | C |
| ANISOU | 3927 | CB | MET | A | 658 | 1247 | 1282 | 1248 | 21 | −29 | 47 | A | C |
| ATOM | 3930 | CG | MET | A | 658 | 16.115 | −11.495 | 20.092 | 1.00 | 11.63 | | A | C |
| ANISOU | 3930 | CG | MET | A | 658 | 1400 | 1521 | 1497 | 82 | −67 | −126 | A | C |
| ATOM | 3933 | SD | MET | A | 658 | 17.671 | −10.658 | 20.635 | 1.00 | 15.26 | | A | S |
| ANISOU | 3933 | SD | MET | A | 658 | 1778 | 1684 | 2337 | 162 | −495 | −211 | A | S |
| ATOM | 3934 | CE | MET | A | 658 | 17.105 | −9.738 | 22.088 | 1.00 | 15.81 | | A | C |
| ANISOU | 3934 | CE | MET | A | 658 | 1841 | 2062 | 2103 | −23 | 31 | 40 | A | C |
| ATOM | 3938 | C | MET | A | 658 | 15.638 | −13.894 | 17.136 | 1.00 | 9.77 | | A | C |
| ANISOU | 3938 | C | MET | A | 658 | 1215 | 1234 | 1261 | 3 | −38 | 51 | A | C |
| ATOM | 3939 | O | MET | A | 658 | 15.151 | −13.440 | 16.106 | 1.00 | 10.18 | | A | O |
| ANISOU | 3939 | O | MET | A | 658 | 1308 | 1171 | 1389 | −26 | −102 | 54 | A | O |
| ATOM | 3940 | N | THR | A | 659 | 16.505 | −14.908 | 17.131 | 1.00 | 10.09 | | A | N |
| ANISOU | 3940 | N | THR | A | 659 | 1266 | 1213 | 1351 | 3 | −82 | 69 | A | N |
| ATOM | 3942 | CA | THR | A | 659 | 17.000 | −15.494 | 15.881 | 1.00 | 11.51 | | A | C |
| ANISOU | 3942 | CA | THR | A | 659 | 1419 | 1436 | 1516 | 37 | 1 | 33 | A | C |
| ATOM | 3944 | CB | THR | A | 659 | 18.166 | −16.476 | 16.089 | 1.00 | 13.24 | | A | C |
| ANISOU | 3944 | CB | THR | A | 659 | 1513 | 1728 | 1789 | 80 | −18 | 3 | A | C |
| ATOM | 3946 | OG1 | THR | A | 659 | 17.735 | −17.581 | 16.856 | 1.00 | 17.70 | | A | O |
| ANISOU | 3946 | OG1 | THR | A | 659 | 2269 | 2217 | 2237 | 110 | 38 | 191 | A | O |
| ATOM | 3948 | CG2 | THR | A | 659 | 19.279 | −15.883 | 16.898 | 1.00 | 12.51 | | A | C |
| ANISOU | 3948 | CG2 | THR | A | 659 | 1570 | 1593 | 1589 | 148 | 10 | −58 | A | C |
| ATOM | 3952 | C | THR | A | 659 | 15.882 | −16.137 | 15.081 | 1.00 | 10.45 | | A | C |
| ANISOU | 3952 | C | THR | A | 659 | 1414 | 1209 | 1347 | 38 | 16 | −37 | A | C |
| ATOM | 3953 | O | THR | A | 659 | 15.909 | −16.094 | 13.858 | 1.00 | 10.83 | | A | O |
| ANISOU | 3953 | O | THR | A | 659 | 1385 | 1405 | 1322 | 80 | −1 | 3 | A | O |
| ATOM | 3954 | N | ARG | A | 660 | 14.900 | −16.721 | 15.777 | 1.00 | 10.63 | | A | N |
| ANISOU | 3954 | N | ARG | A | 660 | 1337 | 1349 | 1351 | 57 | 20 | −64 | A | N |
| ATOM | 3956 | CA | ARG | A | 660 | 13.700 | −17.276 | 15.145 | 1.00 | 11.35 | | A | C |
| ANISOU | 3956 | CA | ARG | A | 660 | 1414 | 1422 | 1475 | −10 | 11 | −18 | A | C |
| ATOM | 3958 | CB | ARG | A | 660 | 12.851 | −18.042 | 16.161 | 1.00 | 12.55 | | A | C |
| ANISOU | 3958 | CB | ARG | A | 660 | 1518 | 1653 | 1596 | −43 | −37 | 2 | A | C |
| ATOM | 3961 | CG | ARG | A | 660 | 13.473 | −19.353 | 16.555 | 1.00 | 15.60 | | A | C |
| ANISOU | 3961 | CG | ARG | A | 660 | 1962 | 1865 | 2097 | −18 | −82 | 44 | A | C |
| ATOM | 3964 | CD | ARG | A | 660 | 12.801 | −19.997 | 17.728 | 1.00 | 19.96 | | A | C |
| ANISOU | 3964 | CD | ARG | A | 660 | 2560 | 2590 | 2432 | 17 | 58 | 55 | A | C |
| ATOM | 3967 | NE | ARG | A | 660 | 11.470 | −20.429 | 17.337 | 1.00 | 22.64 | | A | N |
| ANISOU | 3967 | NE | ARG | A | 660 | 2742 | 2885 | 2975 | −134 | 11 | 88 | A | N |
| ATOM | 3969 | CZ | ARG | A | 660 | 10.390 | −20.450 | 18.129 | 1.00 | 24.17 | | A | C |
| ANISOU | 3969 | CZ | ARG | A | 660 | 2979 | 3153 | 3050 | 8 | 71 | −6 | A | C |
| ATOM | 3970 | NH1 | ARG | A | 660 | 10.448 | −20.106 | 19.423 | 1.00 | 23.56 | | A | N |
| ANISOU | 3970 | NH1 | ARG | A | 660 | 2853 | 2985 | 3111 | −35 | 23 | −91 | A | N |
| ATOM | 3973 | NH2 | ARG | A | 660 | 9.240 | −20.866 | 17.608 | 1.00 | 25.14 | | A | N |
| ANISOU | 3973 | NH2 | ARG | A | 660 | 3066 | 3291 | 3193 | −70 | −15 | −17 | A | N |
| ATOM | 3976 | C | ARG | A | 660 | 12.861 | −16.187 | 14.479 | 1.00 | 10.39 | | A | C |
| ANISOU | 3976 | C | ARG | A | 660 | 1306 | 1255 | 1386 | −38 | 6 | −32 | A | C |
| ATOM | 3977 | O | ARG | A | 660 | 12.333 | −16.373 | 13.387 | 1.00 | 11.12 | | A | O |
| ANISOU | 3977 | O | ARG | A | 660 | 1386 | 1295 | 1540 | −51 | 3 | −122 | A | O |
| ATOM | 3978 | N | CYS | A | 661 | 12.743 | −15.040 | 15.145 | 1.00 | 9.59 | | A | N |
| ANISOU | 3978 | N | CYS | A | 661 | 1241 | 1145 | 1255 | −52 | 45 | −22 | A | N |
| ATOM | 3980 | CA | CYS | A | 661 | 12.046 | −13.902 | 14.564 | 1.00 | 9.30 | | A | C |
| ANISOU | 3980 | CA | CYS | A | 661 | 1063 | 1228 | 1241 | −46 | 23 | 34 | A | C |
| ATOM | 3982 | CB | CYS | A | 661 | 11.912 | −12.785 | 15.587 | 1.00 | 9.52 | | A | C |
| ANISOU | 3982 | CB | CYS | A | 661 | 1113 | 1160 | 1343 | −110 | −33 | 36 | A | C |
| ATOM | 3985 | SG | CYS | A | 661 | 10.905 | −13.173 | 17.035 | 1.00 | 10.38 | | A | S |
| ANISOU | 3985 | SG | CYS | A | 661 | 1174 | 1273 | 1497 | −2 | 34 | 0 | A | S |
| ATOM | 3986 | C | CYS | A | 661 | 12.762 | −13.337 | 13.350 | 1.00 | 9.87 | | A | C |
| ANISOU | 3986 | C | CYS | A | 661 | 1220 | 1284 | 1246 | −12 | −15 | 13 | A | C |
| ATOM | 3987 | O | CYS | A | 661 | 12.141 | −12.648 | 12.551 | 1.00 | 10.25 | | A | O |
| ANISOU | 3987 | O | CYS | A | 661 | 989 | 1459 | 1447 | 80 | −13 | 74 | A | O |
| ATOM | 3988 | N | TRP | A | 662 | 14.069 | −13.607 | 13.254 | 1.00 | 9.56 | | A | N |
| ANISOU | 3988 | N | TRP | A | 662 | 1119 | 1241 | 1270 | 42 | 44 | 49 | A | N |
| ATOM | 3990 | CA | TRP | A | 662 | 14.878 | −13.186 | 12.101 | 1.00 | 8.71 | | A | C |
| ANISOU | 3990 | CA | TRP | A | 662 | 1143 | 1052 | 1114 | 28 | −16 | −23 | A | C |
| ATOM | 3992 | CB | TRP | A | 662 | 16.211 | −12.595 | 12.536 | 1.00 | 8.84 | | A | C |
| ANISOU | 3992 | CB | TRP | A | 662 | 1144 | 1066 | 1147 | −38 | 68 | 10 | A | C |
| ATOM | 3995 | CG | TRP | A | 662 | 16.102 | −11.404 | 13.435 | 1.00 | 9.51 | | A | C |
| ANISOU | 3995 | CG | TRP | A | 662 | 1168 | 1100 | 1346 | 20 | −13 | −20 | A | C |
| ATOM | 3996 | CD1 | TRP | A | 662 | 15.154 | −10.420 | 13.419 | 1.00 | 9.22 | | A | C |
| ANISOU | 3996 | CD1 | TRP | A | 662 | 1067 | 1175 | 1259 | −60 | 36 | 36 | A | C |
| ATOM | 3998 | NE1 | TRP | A | 662 | 15.420 | −9.495 | 14.399 | 1.00 | 9.46 | | A | N |
| ANISOU | 3998 | NE1 | TRP | A | 662 | 1068 | 1133 | 1392 | 115 | 20 | −55 | A | N |
| ATOM | 4000 | CE2 | TRP | A | 662 | 16.518 | −9.904 | 15.105 | 1.00 | 8.11 | | A | C |
| ANISOU | 4000 | CE2 | TRP | A | 662 | 1012 | 881 | 1187 | 12 | 28 | 37 | A | C |
| ATOM | 4001 | CD2 | TRP | A | 662 | 16.975 | −11.091 | 14.516 | 1.00 | 7.35 | | A | C |
| ANISOU | 4001 | CD2 | TRP | A | 662 | 1014 | 780 | 995 | −118 | 148 | 24 | A | C |
| ATOM | 4002 | CE3 | TRP | A | 662 | 18.111 | −11.697 | 15.051 | 1.00 | 8.68 | | A | C |
| ANISOU | 4002 | CE3 | TRP | A | 662 | 1199 | 926 | 1172 | −98 | 39 | 132 | A | C |
| ATOM | 4004 | CZ3 | TRP | A | 662 | 18.733 | −11.119 | 16.123 | 1.00 | 9.60 | | A | C |
| ANISOU | 4004 | CZ3 | TRP | A | 662 | 1434 | 1045 | 1168 | −56 | 51 | 88 | A | C |
| ATOM | 4006 | CH2 | TRP | A | 662 | 18.273 | −9.927 | 16.659 | 1.00 | 9.22 | | A | C |
| ANISOU | 4006 | CH2 | TRP | A | 662 | 1177 | 1161 | 1164 | 7 | −14 | −46 | A | C |
| ATOM | 4008 | CZ2 | TRP | A | 662 | 17.169 | −9.317 | 16.174 | 1.00 | 7.78 | | A | C |
| ANISOU | 4008 | CZ2 | TRP | A | 662 | 1169 | 709 | 1076 | −72 | 94 | 138 | A | C |
| ATOM | 4010 | C | TRP | A | 662 | 15.091 | −14.293 | 11.055 | 1.00 | 9.58 | | A | C |
| ANISOU | 4010 | C | TRP | A | 662 | 1267 | 1208 | 1161 | 17 | 19 | −24 | A | C |
| ATOM | 4011 | O | TRP | A | 662 | 16.022 | −14.238 | 10.263 | 1.00 | 11.04 | | A | O |
| ANISOU | 4011 | O | TRP | A | 662 | 1307 | 1505 | 1380 | −17 | −57 | −62 | A | O |
| ATOM | 4012 | N | ASP | A | 663 | 14.165 | −15.243 | 11.005 | 1.00 | 10.56 | | A | N |
| ANISOU | 4012 | N | ASP | A | 663 | 1377 | 1332 | 1301 | 32 | −22 | −57 | A | N |
| ATOM | 4014 | CA | ASP | A | 663 | 14.176 | −16.193 | 9.910 | 1.00 | 11.21 | | A | C |
| ANISOU | 4014 | CA | ASP | A | 663 | 1435 | 1385 | 1438 | 60 | 73 | −84 | A | C |
| ATOM | 4016 | CB | ASP | A | 663 | 13.095 | −17.242 | 10.080 | 1.00 | 11.68 | | A | C |
| ANISOU | 4016 | CB | ASP | A | 663 | 1559 | 1379 | 1497 | 6 | 29 | −34 | A | C |
| ATOM | 4019 | CG | ASP | A | 663 | 13.384 | −18.471 | 9.271 | 1.00 | 15.73 | | A | C |
| ANISOU | 4019 | CG | ASP | A | 663 | 2147 | 1777 | 2052 | 27 | 92 | −166 | A | C |
| ATOM | 4020 | OD1 | ASP | A | 663 | 13.415 | −18.374 | 8.027 | 1.00 | 19.86 | | A | O |
| ANISOU | 4020 | OD1 | ASP | A | 663 | 2651 | 2427 | 2466 | −53 | 98 | 162 | A | O |
| ATOM | 4021 | OD2 | ASP | A | 663 | 13.604 | −19.579 | 9.797 | 1.00 | 20.97 | | A | O |
| ANISOU | 4021 | OD2 | ASP | A | 663 | 2867 | 2382 | 2717 | 156 | −18 | 183 | A | O |
| ATOM | 4022 | C | ASP | A | 663 | 13.942 | −15.414 | 8.615 | 1.00 | 11.04 | | A | C |
| ANISOU | 4022 | C | ASP | A | 663 | 1362 | 1405 | 1425 | 18 | 64 | −36 | A | C |
| ATOM | 4023 | O | ASP | A | 663 | 13.080 | −14.535 | 8.551 | 1.00 | 10.98 | | A | O |
| ANISOU | 4023 | O | ASP | A | 663 | 1368 | 1406 | 1396 | 145 | 173 | −154 | A | O |
| ATOM | 4024 | N | TYR | A | 664 | 14.746 | −15.668 | 7.595 | 1.00 | 10.94 | | A | N |
| ANISOU | 4024 | N | TYR | A | 664 | 1408 | 1386 | 1359 | 72 | 94 | −108 | A | N |
| ATOM | 4026 | CA | TYR | A | 664 | 14.585 | −14.938 | 6.347 | 1.00 | 10.95 | | A | C |
| ANISOU | 4026 | CA | TYR | A | 664 | 1347 | 1436 | 1378 | 45 | 95 | −37 | A | C |
| ATOM | 4028 | CB | TYR | A | 664 | 15.646 | −15.360 | 5.297 | 1.00 | 11.18 | | A | C |
| ANISOU | 4028 | CB | TYR | A | 664 | 1325 | 1543 | 1380 | 139 | 95 | −100 | A | C |
| ATOM | 4031 | CG | TYR | A | 664 | 15.836 | −14.325 | 4.216 | 1.00 | 11.90 | | A | C |
| ANISOU | 4031 | CG | TYR | A | 664 | 1334 | 1598 | 1588 | 161 | 177 | −86 | A | C |
| ATOM | 4032 | CD1 | TYR | A | 664 | 16.755 | −13.283 | 4.376 | 1.00 | 14.52 | | A | C |
| ANISOU | 4032 | CD1 | TYR | A | 664 | 1791 | 1876 | 1851 | −14 | 159 | 37 | A | C |
| ATOM | 4034 | CE1 | TYR | A | 664 | 16.919 | −12.312 | 3.387 | 1.00 | 15.63 | | A | C |
| ANISOU | 4034 | CE1 | TYR | A | 664 | 1931 | 2039 | 1966 | 46 | 150 | 57 | A | C |
| ATOM | 4036 | CZ | TYR | A | 664 | 16.155 | −12.365 | 2.254 | 1.00 | 16.72 | | A | C |
| ANISOU | 4036 | CZ | TYR | A | 664 | 2101 | 2227 | 2025 | 63 | 130 | 5 | A | C |
| ATOM | 4037 | OH | TYR | A | 664 | 16.340 | −11.393 | 1.280 | 1.00 | 21.10 | | A | O |
| ANISOU | 4037 | OH | TYR | A | 664 | 2890 | 2467 | 2659 | 97 | 209 | 250 | A | O |
| ATOM | 4039 | CE2 | TYR | A | 664 | 15.244 | −13.379 | 2.067 | 1.00 | 16.91 | | A | C |
| ANISOU | 4039 | CE2 | TYR | A | 664 | 2151 | 2226 | 2049 | 94 | 44 | 34 | A | C |
| ATOM | 4041 | CD2 | TYR | A | 664 | 15.081 | −14.355 | 3.046 | 1.00 | 15.74 | | A | C |
| ANISOU | 4041 | CD2 | TYR | A | 664 | 1903 | 2090 | 1985 | 87 | 100 | −38 | A | C |
| ATOM | 4043 | C | TYR | A | 664 | 13.184 | −15.126 | 5.764 | 1.00 | 11.55 | | A | C |
| ANISOU | 4043 | C | TYR | A | 664 | 1432 | 1503 | 1452 | 45 | 72 | −31 | A | C |
| ATOM | 4044 | O | TYR | A | 664 | 12.633 | −14.198 | 5.200 | 1.00 | 11.95 | | A | O |
| ANISOU | 4044 | O | TYR | A | 664 | 1366 | 1631 | 1543 | 58 | 76 | −43 | A | O |
| ATOM | 4045 | N | ASP | A | 665 | 12.641 | −16.335 | 5.884 | 1.00 | 12.57 | | A | N |
| ANISOU | 4045 | N | ASP | A | 665 | 1548 | 1628 | 1600 | −25 | 20 | −86 | A | N |
| ATOM | 4047 | CA | ASP | A | 665 | 11.298 | −16.641 | 5.384 | 1.00 | 13.87 | | A | C |
| ANISOU | 4047 | CA | ASP | A | 665 | 1720 | 1757 | 1792 | −25 | 16 | −53 | A | C |
| ATOM | 4049 | CB | ASP | A | 665 | 11.180 | −18.149 | 5.120 | 1.00 | 15.39 | | A | C |
| ANISOU | 4049 | CB | ASP | A | 665 | 1989 | 1856 | 2003 | −59 | 24 | −51 | A | C |
| ATOM | 4052 | CG | ASP | A | 665 | 9.937 | −18.509 | 4.315 | 1.00 | 18.03 | | A | C |
| ANISOU | 4052 | CG | ASP | A | 665 | 2245 | 2238 | 2366 | −93 | −84 | −45 | A | C |
| ATOM | 4053 | OD1 | ASP | A | 665 | 8.939 | −17.743 | 4.326 | 1.00 | 19.13 | | A | O |
| ANISOU | 4053 | OD1 | ASP | A | 665 | 2399 | 2438 | 2429 | −66 | 91 | −259 | A | O |
| ATOM | 4054 | OD2 | ASP | A | 665 | 9.884 | −19.544 | 3.604 | 1.00 | 21.95 | | A | O |
| ANISOU | 4054 | OD2 | ASP | A | 665 | 2938 | 2574 | 2828 | −76 | 117 | −313 | A | O |
| ATOM | 4055 | C | ASP | A | 665 | 10.245 | −16.208 | 6.398 | 1.00 | 13.43 | | A | C |
| ANISOU | 4055 | C | ASP | A | 665 | 1647 | 1716 | 1739 | −63 | −3 | −50 | A | C |
| ATOM | 4056 | O | ASP | A | 665 | 10.177 | −16.799 | 7.466 | 1.00 | 13.05 | | A | O |
| ANISOU | 4056 | O | ASP | A | 665 | 1623 | 1633 | 1700 | −89 | 135 | −165 | A | O |
| ATOM | 4057 | N | PRO | A | 666 | 9.415 | −15.220 | 6.065 | 1.00 | 13.58 | | A | N |
| ANISOU | 4057 | N | PRO | A | 666 | 1661 | 1726 | 1773 | −70 | 2 | −67 | A | N |
| ATOM | 4058 | CA | PRO | A | 666 | 8.384 | −14.749 | 7.002 | 1.00 | 14.08 | | A | C |
| ANISOU | 4058 | CA | PRO | A | 666 | 1785 | 1800 | 1765 | −58 | 7 | −86 | A | C |
| ATOM | 4060 | CB | PRO | A | 666 | 7.630 | −13.674 | 6.206 | 1.00 | 14.61 | | A | C |
| ANISOU | 4060 | CB | PRO | A | 666 | 1813 | 1886 | 1849 | −23 | 40 | −36 | A | C |
| ATOM | 4063 | CG | PRO | A | 666 | 8.031 | −13.821 | 4.807 | 1.00 | 14.68 | | A | C |
| ANISOU | 4063 | CG | PRO | A | 666 | 1960 | 1798 | 1818 | 69 | −5 | 9 | A | C |
| ATOM | 4066 | CD | PRO | A | 666 | 9.358 | −14.493 | 4.784 | 1.00 | 13.93 | | A | C |
| ANISOU | 4066 | CD | PRO | A | 666 | 1622 | 1871 | 1800 | −95 | 29 | −41 | A | C |
| ATOM | 4069 | C | PRO | A | 666 | 7.444 | −15.841 | 7.477 | 1.00 | 14.93 | | A | C |
| ANISOU | 4069 | C | PRO | A | 666 | 1843 | 1956 | 1872 | −87 | 64 | −64 | A | C |
| ATOM | 4070 | O | PRO | A | 666 | 6.998 | −15.834 | 8.615 | 1.00 | 13.71 | | A | O |
| ANISOU | 4070 | O | PRO | A | 666 | 1765 | 1733 | 1708 | −155 | 120 | −111 | A | O |
| ATOM | 4071 | N | SER | A | 667 | 7.168 | −16.808 | 6.611 | 1.00 | 15.82 | | A | N |
| ANISOU | 4071 | N | SER | A | 667 | 2055 | 2031 | 1924 | −109 | 69 | −104 | A | N |
| ATOM | 4073 | CA | SER | A | 667 | 6.258 | −17.902 | 6.962 | 1.00 | 17.09 | | A | C |
| ANISOU | 4073 | CA | SER | A | 667 | 2157 | 2139 | 2197 | −69 | 67 | −29 | A | C |
| ATOM | 4075 | CB | SER | A | 667 | 5.990 | −18.807 | 5.745 | 1.00 | 17.46 | | A | C |
| ANISOU | 4075 | CB | SER | A | 667 | 2184 | 2259 | 2190 | −132 | −2 | −12 | A | C |
| ATOM | 4078 | OG | SER | A | 667 | 5.326 | −18.081 | 4.731 | 1.00 | 21.69 | | A | O |
| ANISOU | 4078 | OG | SER | A | 667 | 2671 | 2769 | 2801 | −64 | −20 | 101 | A | O |
| ATOM | 4080 | C | SER | A | 667 | 6.758 | −18.748 | 8.116 | 1.00 | 16.80 | | A | C |
| ANISOU | 4080 | C | SER | A | 667 | 2118 | 2078 | 2185 | −36 | 57 | −37 | A | C |
| ATOM | 4081 | O | SER | A | 667 | 5.958 | −19.438 | 8.742 | 1.00 | 18.68 | | A | O |
| ANISOU | 4081 | O | SER | A | 667 | 2284 | 2327 | 2485 | −104 | 145 | −21 | A | O |
| ATOM | 4082 | N | ASP | A | 668 | 8.066 | −18.697 | 8.405 | 1.00 | 16.50 | | A | N |
| ANISOU | 4082 | N | ASP | A | 668 | 2110 | 2001 | 2156 | −22 | 90 | −75 | A | N |
| ATOM | 4084 | CA | ASP | A | 668 | 8.689 | −19.497 | 9.442 | 1.00 | 16.07 | | A | C |
| ANISOU | 4084 | CA | ASP | A | 668 | 2049 | 1985 | 2071 | −15 | 71 | −56 | A | C |
| ATOM | 4086 | CB | ASP | A | 668 | 10.047 | −20.026 | 8.963 | 1.00 | 17.36 | | A | C |
| ANISOU | 4086 | CB | ASP | A | 668 | 2171 | 2170 | 2254 | −5 | 107 | −18 | A | C |
| ATOM | 4089 | CG | ASP | A | 668 | 9.928 | −20.986 | 7.786 | 1.00 | 20.84 | | A | C |
| ANISOU | 4089 | CG | ASP | A | 668 | 2672 | 2615 | 2630 | −11 | 27 | −166 | A | C |
| ATOM | 4090 | OD1 | ASP | A | 668 | 8.843 | −21.546 | 7.549 | 1.00 | 23.33 | | A | O |
| ANISOU | 4090 | OD1 | ASP | A | 668 | 2944 | 2843 | 3076 | −59 | 43 | −184 | A | O |
| ATOM | 4091 | OD2 | ASP | A | 668 | 10.891 | −21.222 | 7.043 | 1.00 | 23.92 | | A | O |
| ANISOU | 4091 | OD2 | ASP | A | 668 | 3031 | 3104 | 2953 | 55 | 199 | −250 | A | O |
| ATOM | 4092 | C | ASP | A | 668 | 8.917 | −18.758 | 10.749 | 1.00 | 14.67 | | A | C |
| ANISOU | 4092 | C | ASP | A | 668 | 1864 | 1792 | 1914 | −26 | 79 | 5 | A | C |
| ATOM | 4093 | O | ASP | A | 668 | 9.392 | −19.325 | 11.728 | 1.00 | 15.36 | | A | O |
| ANISOU | 4093 | O | ASP | A | 668 | 2059 | 1636 | 2140 | 56 | 125 | 55 | A | O |
| ATOM | 4094 | N | ARG | A | 669 | 8.601 | −17.477 | 10.771 | 1.00 | 12.20 | | A | N |
| ANISOU | 4094 | N | ARG | A | 669 | 1572 | 1520 | 1541 | −8 | 130 | −27 | A | N |
| ATOM | 4096 | CA | ARG | A | 669 | 8.714 | −16.724 | 12.017 | 1.00 | 11.14 | | A | C |
| ANISOU | 4096 | CA | ARG | A | 669 | 1360 | 1425 | 1445 | −26 | 65 | −12 | A | C |
| ATOM | 4098 | CB | ARG | A | 669 | 8.675 | −15.232 | 11.709 | 1.00 | 10.46 | | A | C |
| ANISOU | 4098 | CB | ARG | A | 669 | 1219 | 1353 | 1401 | −24 | 104 | −3 | A | C |
| ATOM | 4101 | CG | ARG | A | 669 | 9.864 | −14.751 | 10.904 | 1.00 | 9.70 | | A | C |
| ANISOU | 4101 | CG | ARG | A | 669 | 1123 | 1216 | 1346 | −41 | 82 | −16 | A | C |
| ATOM | 4104 | CD | ARG | A | 669 | 9.824 | −13.310 | 10.535 | 1.00 | 9.29 | | A | C |
| ANISOU | 4104 | CD | ARG | A | 669 | 1160 | 1187 | 1180 | 8 | 41 | −16 | A | C |
| ATOM | 4107 | NE | ARG | A | 669 | 10.704 | −13.141 | 9.382 | 1.00 | 9.73 | | A | N |
| ANISOU | 4107 | NE | ARG | A | 669 | 1108 | 1304 | 1282 | 47 | 121 | 17 | A | N |
| ATOM | 4109 | CZ | ARG | A | 669 | 10.517 | −12.283 | 8.391 | 1.00 | 8.76 | | A | C |
| ANISOU | 4109 | CZ | ARG | A | 669 | 976 | 1136 | 1215 | −29 | 55 | −8 | A | C |
| ATOM | 4110 | NH1 | ARG | A | 669 | 9.545 | −11.374 | 8.457 | 1.00 | 9.89 | | A | N |
| ANISOU | 4110 | NH1 | ARG | A | 669 | 1257 | 1069 | 1429 | 107 | 31 | 79 | A | N |
| ATOM | 4113 | NH2 | ARG | A | 669 | 11.321 | −12.318 | 7.331 | 1.00 | 9.41 | | A | N |
| ANISOU | 4113 | NH2 | ARG | A | 669 | 958 | 1356 | 1259 | 67 | −25 | 14 | A | N |
| ATOM | 4116 | C | ARG | A | 669 | 7.560 | −17.057 | 12.966 | 1.00 | 11.24 | | A | C |
| ANISOU | 4116 | C | ARG | A | 669 | 1381 | 1382 | 1505 | −24 | 40 | 46 | A | C |
| ATOM | 4117 | O | ARG | A | 669 | 6.462 | −17.362 | 12.504 | 1.00 | 12.43 | | A | O |
| ANISOU | 4117 | O | ARG | A | 669 | 1429 | 1678 | 1616 | −7 | −7 | 36 | A | O |
| ATOM | 4118 | N | PRO | A | 670 | 7.777 | −16.932 | 14.278 | 1.00 | 10.78 | | A | N |
| ANISOU | 4118 | N | PRO | A | 670 | 1358 | 1304 | 1433 | −16 | 44 | 24 | A | N |
| ATOM | 4119 | CA | PRO | A | 670 | 6.693 | −17.121 | 15.258 | 1.00 | 11.05 | | A | C |
| ANISOU | 4119 | CA | PRO | A | 670 | 1382 | 1306 | 1508 | 94 | 74 | 11 | A | C |
| ATOM | 4121 | CB | PRO | A | 670 | 7.395 | −16.954 | 16.603 | 1.00 | 12.51 | | A | C |
| ANISOU | 4121 | CB | PRO | A | 670 | 1574 | 1557 | 1622 | 93 | 110 | 106 | A | C |
| ATOM | 4124 | CG | PRO | A | 670 | 8.848 | −17.065 | 16.308 | 1.00 | 14.02 | | A | C |
| ANISOU | 4124 | CG | PRO | A | 670 | 1667 | 1900 | 1757 | 32 | 29 | 37 | A | C |
| ATOM | 4127 | CD | PRO | A | 670 | 9.038 | −16.566 | 14.941 | 1.00 | 11.84 | | A | C |
| ANISOU | 4127 | CD | PRO | A | 670 | 1398 | 1492 | 1609 | 58 | 57 | −14 | A | C |
| ATOM | 4130 | C | PRO | A | 670 | 5.563 | −16.085 | 15.124 | 1.00 | 10.64 | | A | C |
| ANISOU | 4130 | C | PRO | A | 670 | 1390 | 1262 | 1391 | 77 | 10 | 61 | A | C |
| ATOM | 4131 | O | PRO | A | 670 | 5.789 | −14.991 | 14.623 | 1.00 | 10.77 | | A | O |
| ANISOU | 4131 | O | PRO | A | 670 | 1372 | 1213 | 1505 | 26 | 100 | 103 | A | O |
| ATOM | 4132 | N | ARG | A | 671 | 4.386 | −16.459 | 15.610 | 1.00 | 9.98 | | A | N |
| ANISOU | 4132 | N | ARG | A | 671 | 1397 | 1118 | 1277 | 42 | 43 | 16 | A | N |
| ATOM | 4134 | CA | ARG | A | 671 | 3.287 | −15.549 | 15.845 | 1.00 | 9.83 | | A | C |
| ANISOU | 4134 | CA | ARG | A | 671 | 1239 | 1273 | 1221 | 17 | 15 | 41 | A | C |
| ATOM | 4136 | CB | ARG | A | 671 | 2.003 | −16.327 | 16.099 | 1.00 | 11.36 | | A | C |
| ANISOU | 4136 | CB | ARG | A | 671 | 1424 | 1436 | 1456 | −26 | 58 | −32 | A | C |
| ATOM | 4139 | CG | ARG | A | 671 | 1.492 | −17.209 | 14.990 | 1.00 | 11.68 | | A | C |
| ANISOU | 4139 | CG | ARG | A | 671 | 1487 | 1442 | 1507 | −7 | −2 | −62 | A | C |
| ATOM | 4142 | CD | ARG | A | 671 | 0.265 | −17.961 | 15.456 | 1.00 | 13.47 | | A | C |
| ANISOU | 4142 | CD | ARG | A | 671 | 1545 | 1847 | 1724 | −108 | −54 | −90 | A | C |
| ATOM | 4145 | NE | ARG | A | 671 | −0.454 | −18.701 | 14.418 | 1.00 | 15.63 | | A | N |
| ANISOU | 4145 | NE | ARG | A | 671 | 2178 | 2005 | 1754 | −61 | −88 | −169 | A | N |
| ATOM | 4147 | CZ | ARG | A | 671 | −0.352 | −20.004 | 14.198 | 1.00 | 16.74 | | A | C |
| ANISOU | 4147 | CZ | ARG | A | 671 | 2188 | 2083 | 2090 | 38 | −77 | −75 | A | C |
| ATOM | 4148 | NH1 | ARG | A | 671 | 0.472 | −20.772 | 14.900 | 1.00 | 16.43 | | A | N |
| ANISOU | 4148 | NH1 | ARG | A | 671 | 2378 | 2055 | 1808 | −56 | −63 | −72 | A | N |
| ATOM | 4151 | NH2 | ARG | A | 671 | −1.099 | −20.541 | 13.244 | 1.00 | 19.05 | | A | N |
| ANISOU | 4151 | NH2 | ARG | A | 671 | 2530 | 2437 | 2270 | −95 | −158 | −187 | A | N |
| ATOM | 4154 | C | ARG | A | 671 | 3.548 | −14.728 | 17.094 | 1.00 | 9.52 | | A | C |
| ANISOU | 4154 | C | ARG | A | 671 | 1255 | 1156 | 1204 | −23 | 42 | 55 | A | C |
| ATOM | 4155 | O | ARG | A | 671 | 4.253 | −15.171 | 17.987 | 1.00 | 9.26 | | A | O |
| ANISOU | 4155 | O | ARG | A | 671 | 1265 | 1053 | 1199 | −19 | 18 | 81 | A | O |
| ATOM | 4156 | N | PHE | A | 672 | 2.968 | −13.545 | 17.175 | 1.00 | 8.86 | | A | N |
| ANISOU | 4156 | N | PHE | A | 672 | 1187 | 1201 | 979 | 47 | 26 | 108 | A | N |
| ATOM | 4158 | CA | PHE | A | 672 | 3.056 | −12.781 | 18.411 | 1.00 | 9.06 | | A | C |
| ANISOU | 4158 | CA | PHE | A | 672 | 1203 | 1150 | 1088 | 1 | −35 | 5 | A | C |
| ATOM | 4160 | CB | PHE | A | 672 | 2.491 | −11.371 | 18.298 | 1.00 | 8.73 | | A | C |
| ANISOU | 4160 | CB | PHE | A | 672 | 1134 | 1162 | 1019 | −20 | −23 | 15 | A | C |
| ATOM | 4163 | CG | PHE | A | 672 | 3.413 | −10.391 | 17.617 | 1.00 | 8.48 | | A | C |
| ANISOU | 4163 | CG | PHE | A | 672 | 1018 | 1176 | 1028 | 18 | 64 | −38 | A | C |
| ATOM | 4164 | CD1 | PHE | A | 672 | 4.590 | −9.994 | 18.243 | 1.00 | 7.89 | | A | C |
| ANISOU | 4164 | CD1 | PHE | A | 672 | 930 | 1064 | 1001 | 43 | −52 | 175 | A | C |
| ATOM | 4166 | CE1 | PHE | A | 672 | 5.453 | −9.071 | 17.647 | 1.00 | 8.09 | | A | C |
| ANISOU | 4166 | CE1 | PHE | A | 672 | 851 | 1131 | 1090 | 22 | 51 | 83 | A | C |
| ATOM | 4168 | CZ | PHE | A | 672 | 5.128 | −8.537 | 16.440 | 1.00 | 8.28 | | A | C |
| ANISOU | 4168 | CZ | PHE | A | 672 | 987 | 1081 | 1078 | 48 | 124 | −9 | A | C |
| ATOM | 4170 | CE2 | PHE | A | 672 | 3.961 | −8.912 | 15.811 | 1.00 | 7.94 | | A | C |
| ANISOU | 4170 | CE2 | PHE | A | 672 | 1007 | 1214 | 794 | 71 | 26 | 65 | A | C |
| ATOM | 4172 | CD2 | PHE | A | 672 | 3.093 | −9.839 | 16.405 | 1.00 | 8.53 | | A | C |
| ANISOU | 4172 | CD2 | PHE | A | 672 | 900 | 1101 | 1240 | 155 | 72 | 67 | A | C |
| ATOM | 4174 | C | PHE | A | 672 | 2.434 | −13.503 | 19.608 | 1.00 | 9.51 | | A | C |
| ANISOU | 4174 | C | PHE | A | 672 | 1260 | 1187 | 1165 | −75 | −10 | 25 | A | C |
| ATOM | 4175 | O | PHE | A | 672 | 2.931 | −13.344 | 20.712 | 1.00 | 9.70 | | A | O |
| ANISOU | 4175 | O | PHE | A | 672 | 1391 | 1234 | 1058 | 22 | 24 | 64 | A | O |
| ATOM | 4176 | N | THR | A | 673 | 1.365 | −14.279 | 19.415 | 1.00 | 10.26 | | A | N |
| ANISOU | 4176 | N | THR | A | 673 | 1338 | 1385 | 1174 | −24 | −79 | 13 | A | N |
| ATOM | 4178 | CA | THR | A | 673 | 0.796 | −15.017 | 20.538 | 1.00 | 11.04 | | A | C |
| ANISOU | 4178 | CA | THR | A | 673 | 1435 | 1416 | 1341 | −90 | −18 | −35 | A | C |
| ATOM | 4180 | CB | THR | A | 673 | −0.429 | −15.818 | 20.113 | 1.00 | 11.04 | | A | C |
| ANISOU | 4180 | CB | THR | A | 673 | 1462 | 1418 | 1314 | −124 | 47 | −10 | A | C |
| ATOM | 4182 | OG1 | THR | A | 673 | −0.113 | −16.572 | 18.929 | 1.00 | 12.62 | | A | O |
| ANISOU | 4182 | OG1 | THR | A | 673 | 1749 | 1703 | 1342 | −91 | −1 | −63 | A | O |
| ATOM | 4184 | CG2 | THR | A | 673 | −1.572 | −14.927 | 19.794 | 1.00 | 14.01 | | A | C |
| ANISOU | 4184 | CG2 | THR | A | 673 | 1794 | 1912 | 1616 | −96 | −81 | −17 | A | C |
| ATOM | 4188 | C | THR | A | 673 | 1.832 | −15.982 | 21.106 | 1.00 | 10.65 | | A | C |
| ANISOU | 4188 | C | THR | A | 673 | 1422 | 1377 | 1247 | −87 | −39 | −32 | A | C |
| ATOM | 4189 | O | THR | A | 673 | 1.945 | −16.165 | 22.331 | 1.00 | 11.28 | | A | O |
| ANISOU | 4189 | O | THR | A | 673 | 1720 | 1470 | 1095 | −84 | −114 | −35 | A | O |
| ATOM | 4190 | N | GLU | A | 674 | 2.581 | −16.610 | 20.215 | 1.00 | 10.28 | | A | N |
| ANISOU | 4190 | N | GLU | A | 674 | 1318 | 1415 | 1170 | −66 | −60 | −48 | A | N |
| ATOM | 4192 | CA | GLU | A | 674 | 3.604 | −17.558 | 20.601 | 1.00 | 9.55 | | A | C |
| ANISOU | 4192 | CA | GLU | A | 674 | 1286 | 1171 | 1169 | −95 | −17 | 49 | A | C |
| ATOM | 4194 | CB | GLU | A | 674 | 4.089 | −18.337 | 19.379 | 1.00 | 9.92 | | A | C |
| ANISOU | 4194 | CB | GLU | A | 674 | 1261 | 1287 | 1218 | 11 | −34 | 76 | A | C |
| ATOM | 4197 | CG | GLU | A | 674 | 2.987 | −19.163 | 18.766 | 1.00 | 10.67 | | A | C |
| ANISOU | 4197 | CG | GLU | A | 674 | 1529 | 1286 | 1240 | −121 | −59 | 77 | A | C |
| ATOM | 4200 | CD | GLU | A | 674 | 3.274 | −19.715 | 17.377 | 1.00 | 13.11 | | A | C |
| ANISOU | 4200 | CD | GLU | A | 674 | 1664 | 1734 | 1583 | −101 | 61 | −153 | A | C |
| ATOM | 4201 | OE1 | GLU | A | 674 | 4.217 | −19.266 | 16.690 | 1.00 | 13.40 | | A | O |
| ANISOU | 4201 | OE1 | GLU | A | 674 | 2084 | 1432 | 1573 | −102 | 184 | −21 | A | O |
| ATOM | 4202 | OE2 | GLU | A | 674 | 2.507 | −20.632 | 16.978 | 1.00 | 13.86 | | A | O |
| ANISOU | 4202 | OE2 | GLU | A | 674 | 2025 | 1710 | 1529 | −255 | −34 | −61 | A | O |
| ATOM | 4203 | C | GLU | A | 674 | 4.764 | −16.849 | 21.313 | 1.00 | 10.35 | | A | C |
| ANISOU | 4203 | C | GLU | A | 674 | 1439 | 1212 | 1280 | −51 | −106 | 36 | A | C |
| ATOM | 4204 | O | GLU | A | 674 | 5.255 | −17.336 | 22.338 | 1.00 | 10.34 | | A | O |
| ANISOU | 4204 | O | GLU | A | 674 | 1523 | 1195 | 1208 | −118 | −224 | −58 | A | O |
| ATOM | 4205 | N | LEU | A | 675 | 5.140 | −15.671 | 20.820 | 1.00 | 9.65 | | A | N |
| ANISOU | 4205 | N | LEU | A | 675 | 1316 | 1240 | 1110 | −87 | −64 | 54 | A | N |
| ATOM | 4207 | CA | LEU | A | 675 | 6.188 | −14.882 | 21.455 | 1.00 | 9.88 | | A | C |
| ANISOU | 4207 | CA | LEU | A | 675 | 1278 | 1213 | 1262 | −26 | 0 | 16 | A | C |
| ATOM | 4209 | CB | LEU | A | 675 | 6.548 | −13.672 | 20.608 | 1.00 | 9.99 | | A | C |
| ANISOU | 4209 | CB | LEU | A | 675 | 1219 | 1193 | 1381 | −28 | −25 | 109 | A | C |
| ATOM | 4212 | CG | LEU | A | 675 | 7.239 | −13.975 | 19.315 | 1.00 | 11.68 | | A | C |
| ANISOU | 4212 | CG | LEU | A | 675 | 1357 | 1474 | 1605 | 1 | −7 | −59 | A | C |
| ATOM | 4214 | CD1 | LEU | A | 675 | 7.406 | −12.708 | 18.480 | 1.00 | 11.63 | | A | C |
| ANISOU | 4214 | CD1 | LEU | A | 675 | 1336 | 1609 | 1471 | 30 | 49 | −12 | A | C |
| ATOM | 4218 | CD2 | LEU | A | 675 | 8.602 | −14.677 | 19.558 | 1.00 | 13.00 | | A | C |
| ANISOU | 4218 | CD2 | LEU | A | 675 | 1621 | 1429 | 1889 | 192 | 111 | 39 | A | C |
| ATOM | 4222 | C | LEU | A | 675 | 5.784 | −14.409 | 22.837 | 1.00 | 9.06 | | A | C |
| ANISOU | 4222 | C | LEU | A | 675 | 1220 | 1011 | 1211 | −29 | −10 | −29 | A | C |
| ATOM | 4223 | O | LEU | A | 675 | 6.635 | −14.391 | 23.729 | 1.00 | 9.64 | | A | O |
| ANISOU | 4223 | O | LEU | A | 675 | 1264 | 999 | 1400 | −121 | −95 | −31 | A | O |
| ATOM | 4224 | N | VAL | A | 676 | 4.515 | −14.065 | 23.036 | 1.00 | 8.74 | | A | N |
| ANISOU | 4224 | N | VAL | A | 676 | 1183 | 1015 | 1122 | −11 | −86 | 12 | A | N |
| ATOM | 4226 | CA | VAL | A | 676 | 4.054 | −13.684 | 24.378 | 1.00 | 9.18 | | A | C |
| ANISOU | 4226 | CA | VAL | A | 676 | 1202 | 1115 | 1170 | 14 | 0 | 7 | A | C |
| ATOM | 4228 | CB | VAL | A | 676 | 2.565 | −13.318 | 24.424 | 1.00 | 9.40 | | A | C |
| ANISOU | 4228 | CB | VAL | A | 676 | 1206 | 1125 | 1241 | −70 | −15 | 4 | A | C |
| ATOM | 4230 | CG1 | VAL | A | 676 | 2.078 | −13.219 | 25.874 | 1.00 | 9.87 | | A | C |
| ANISOU | 4230 | CG1 | VAL | A | 676 | 1297 | 1054 | 1396 | −30 | 27 | −38 | A | C |
| ATOM | 4234 | CG2 | VAL | A | 676 | 2.296 | −12.008 | 23.707 | 1.00 | 10.69 | | A | C |
| ANISOU | 4234 | CG2 | VAL | A | 676 | 1381 | 1261 | 1419 | 60 | 27 | 19 | A | C |
| ATOM | 4238 | C | VAL | A | 676 | 4.329 | −14.854 | 25.331 | 1.00 | 9.61 | | A | C |
| ANISOU | 4238 | C | VAL | A | 676 | 1309 | 1158 | 1182 | −11 | −20 | −41 | A | C |
| ATOM | 4239 | O | VAL | A | 676 | 4.869 | −14.665 | 26.407 | 1.00 | 10.06 | | A | O |
| ANISOU | 4239 | O | VAL | A | 676 | 1430 | 1231 | 1159 | 25 | −91 | −130 | A | O |
| ATOM | 4240 | N | CYS | A | 677 | 3.982 | −16.072 | 24.934 | 1.00 | 9.35 | | A | N |
| ANISOU | 4240 | N | CYS | A | 677 | 1252 | 1125 | 1172 | 1 | 17 | −97 | A | N |
| ATOM | 4242 | CA | CYS | A | 677 | 4.188 | −17.218 | 25.804 | 1.00 | 10.25 | | A | C |
| ANISOU | 4242 | CA | CYS | A | 677 | 1352 | 1296 | 1245 | −50 | −30 | −78 | A | C |
| ATOM | 4244 | CB | CYS | A | 677 | 3.609 | −18.447 | 25.185 | 1.00 | 10.64 | | A | C |
| ANISOU | 4244 | CB | CYS | A | 677 | 1398 | 1408 | 1236 | −58 | −63 | −72 | A | C |
| ATOM | 4247 | SG | ACYS | A | 677 | 1.829 | −18.464 | 25.115 | 0.50 | 13.52 | | A | S |
| ANISOU | 4247 | SG | ACYS | A | 677 | 1551 | 1729 | 1857 | 0 | −17 | −204 | A | S |
| ATOM | 4248 | SG | BCYS | A | 677 | 3.228 | −19.741 | 26.364 | 0.50 | 10.56 | | A | S |
| ANISOU | 4248 | SG | BCYS | A | 677 | 1390 | 1399 | 1222 | −109 | 107 | −235 | A | S |
| ATOM | 4249 | C | CYS | A | 677 | 5.666 | −17.473 | 26.061 | 1.00 | 9.43 | | A | C |
| ANISOU | 4249 | C | CYS | A | 677 | 1285 | 1179 | 1117 | 12 | −21 | −31 | A | C |
| ATOM | 4250 | O | CYS | A | 677 | 6.058 | −17.707 | 27.201 | 1.00 | 10.05 | | A | O |
| ANISOU | 4250 | O | CYS | A | 677 | 1414 | 1194 | 1210 | −153 | −252 | −36 | A | O |
| ATOM | 4251 | N | SER | A | 678 | 6.486 | −17.383 | 25.008 | 1.00 | 8.36 | | A | N |
| ANISOU | 4251 | N | SER | A | 678 | 1152 | 1066 | 955 | −35 | −75 | 27 | A | N |
| ATOM | 4253 | CA | SER | A | 678 | 7.915 | −17.600 | 25.138 | 1.00 | 9.06 | | A | C |
| ANISOU | 4253 | CA | SER | A | 678 | 1204 | 1113 | 1124 | −61 | −69 | −12 | A | C |
| ATOM | 4255 | CB | SER | A | 678 | 8.584 | −17.543 | 23.793 | 1.00 | 9.26 | | A | C |
| ANISOU | 4255 | CB | SER | A | 678 | 1275 | 1086 | 1157 | −61 | −66 | −68 | A | C |
| ATOM | 4258 | OG | SER | A | 678 | 8.180 | −18.653 | 23.002 | 1.00 | 12.08 | | A | O |
| ANISOU | 4258 | OG | SER | A | 678 | 1647 | 1513 | 1429 | −130 | −64 | −419 | A | O |
| ATOM | 4260 | C | SER | A | 678 | 8.534 | −16.552 | 26.054 | 1.00 | 9.50 | | A | C |
| ANISOU | 4260 | C | SER | A | 678 | 1252 | 1205 | 1150 | −13 | −138 | −17 | A | C |
| ATOM | 4261 | O | SER | A | 678 | 9.335 | −16.884 | 26.933 | 1.00 | 9.54 | | A | O |
| ANISOU | 4261 | O | SER | A | 678 | 1226 | 1317 | 1081 | 29 | −338 | 33 | A | O |
| ATOM | 4262 | N | LEU | A | 679 | 8.178 | −15.298 | 25.863 | 1.00 | 9.02 | | A | N |
| ANISOU | 4262 | N | LEU | A | 679 | 1184 | 1158 | 1083 | −49 | −186 | −6 | A | N |
| ATOM | 4264 | CA | LEU | A | 679 | 8.752 | −14.229 | 26.679 | 1.00 | 9.18 | | A | C |
| ANISOU | 4264 | CA | LEU | A | 679 | 1268 | 1121 | 1097 | −22 | −76 | −27 | A | C |
| ATOM | 4266 | CB | LEU | A | 679 | 8.417 | −12.860 | 26.114 | 1.00 | 9.07 | | A | C |
| ANISOU | 4266 | CB | LEU | A | 679 | 1150 | 1116 | 1178 | −20 | −80 | −62 | A | C |
| ATOM | 4269 | CG | LEU | A | 679 | 9.285 | −12.455 | 24.942 | 1.00 | 11.44 | | A | C |
| ANISOU | 4269 | CG | LEU | A | 679 | 1495 | 1415 | 1435 | −16 | −14 | 62 | A | C |
| ATOM | 4271 | CD1 | LEU | A | 679 | 8.711 | −11.208 | 24.280 | 1.00 | 10.61 | | A | C |
| ANISOU | 4271 | CD1 | LEU | A | 679 | 1316 | 1465 | 1250 | 45 | −25 | 105 | A | C |
| ATOM | 4275 | CD2 | LEU | A | 679 | 10.694 | −12.242 | 25.408 | 1.00 | 14.76 | | A | C |
| ANISOU | 4275 | CD2 | LEU | A | 679 | 1710 | 1899 | 1997 | −16 | −45 | 124 | A | C |
| ATOM | 4279 | C | LEU | A | 679 | 8.261 | −14.341 | 28.110 | 1.00 | 9.19 | | A | C |
| ANISOU | 4279 | C | LEU | A | 679 | 1199 | 1164 | 1128 | −49 | −33 | −33 | A | C |
| ATOM | 4280 | O | LEU | A | 679 | 9.007 | −14.024 | 29.021 | 1.00 | 9.17 | | A | O |
| ANISOU | 4280 | O | LEU | A | 679 | 1350 | 1097 | 1034 | −88 | −148 | −73 | A | O |
| ATOM | 4281 | N | SER | A | 680 | 7.040 | −14.831 | 28.318 | 1.00 | 9.53 | | A | N |
| ANISOU | 4281 | N | SER | A | 680 | 1264 | 1260 | 1096 | −104 | −32 | −73 | A | N |
| ATOM | 4283 | CA | SER | A | 680 | 6.542 | −15.069 | 29.662 | 1.00 | 9.87 | | A | C |
| ANISOU | 4283 | CA | SER | A | 680 | 1307 | 1230 | 1212 | −48 | −17 | 4 | A | C |
| ATOM | 4285 | CB | SER | A | 680 | 5.084 | −15.519 | 29.607 | 1.00 | 10.57 | | A | C |
| ANISOU | 4285 | CB | SER | A | 680 | 1323 | 1383 | 1309 | −43 | −9 | −25 | A | C |
| ATOM | 4288 | OG | SER | A | 680 | 4.254 | −14.443 | 29.194 | 1.00 | 12.61 | | A | O |
| ANISOU | 4288 | OG | SER | A | 680 | 1503 | 1645 | 1642 | −154 | 9 | 112 | A | O |
| ATOM | 4290 | C | SER | A | 680 | 7.411 | −16.120 | 30.370 | 1.00 | 9.74 | | A | C |
| ANISOU | 4290 | C | SER | A | 680 | 1253 | 1259 | 1187 | −47 | −65 | −27 | A | C |
| ATOM | 4291 | O | SER | A | 680 | 7.721 | −15.995 | 31.552 | 1.00 | 9.65 | | A | O |
| ANISOU | 4291 | O | SER | A | 680 | 1260 | 1259 | 1145 | −176 | −159 | 11 | A | O |
| ATOM | 4292 | N | ASP | A | 681 | 7.832 | −17.128 | 29.622 | 1.00 | 10.22 | | A | N |
| ANISOU | 4292 | N | ASP | A | 681 | 1311 | 1310 | 1260 | −95 | −74 | −21 | A | N |
| ATOM | 4294 | CA | ASP | A | 681 | 8.692 | −18.143 | 30.160 | 1.00 | 10.85 | | A | C |
| ANISOU | 4294 | CA | ASP | A | 681 | 1413 | 1376 | 1330 | −58 | 15 | −23 | A | C |
| ATOM | 4296 | CB | ASP | A | 681 | 8.727 | −19.322 | 29.206 | 1.00 | 11.85 | | A | C |
| ANISOU | 4296 | CB | ASP | A | 681 | 1502 | 1506 | 1493 | −82 | −23 | −74 | A | C |
| ATOM | 4299 | CG | ASP | A | 681 | 9.338 | −20.518 | 29.799 | 1.00 | 16.25 | | A | C |
| ANISOU | 4299 | CG | ASP | A | 681 | 2172 | 1871 | 2130 | −27 | −50 | 15 | A | C |
| ATOM | 4300 | OD1 | ASP | A | 681 | 9.067 | −20.848 | 30.985 | 1.00 | 19.17 | | A | O |
| ANISOU | 4300 | OD1 | ASP | A | 681 | 2825 | 2056 | 2401 | −98 | 37 | 95 | A | O |
| ATOM | 4301 | OD2 | ASP | A | 681 | 10.135 | −21.197 | 29.137 | 1.00 | 22.71 | | A | O |
| ANISOU | 4301 | OD2 | ASP | A | 681 | 2860 | 2896 | 2873 | 100 | 287 | −125 | A | O |
| ATOM | 4302 | C | ASP | A | 681 | 10.093 | −17.582 | 30.420 | 1.00 | 9.83 | | A | C |
| ANISOU | 4302 | C | ASP | A | 681 | 1291 | 1229 | 1214 | −33 | −9 | 3 | A | C |
| ATOM | 4303 | O | ASP | A | 681 | 10.697 | −17.870 | 31.453 | 1.00 | 9.52 | | A | O |
| ANISOU | 4303 | O | ASP | A | 681 | 1465 | 1109 | 1043 | −208 | −94 | −14 | A | O |
| ATOM | 4304 | N | VAL | A | 682 | 10.618 | −16.755 | 29.524 | 1.00 | 9.45 | | A | N |
| ANISOU | 4304 | N | VAL | A | 682 | 1302 | 1248 | 1041 | −17 | −59 | −27 | A | N |
| ATOM | 4306 | CA | VAL | A | 682 | 11.932 | −16.182 | 29.763 | 1.00 | 9.70 | | A | C |
| ANISOU | 4306 | CA | VAL | A | 682 | 1237 | 1272 | 1178 | −1 | 0 | −22 | A | C |
| ATOM | 4308 | CB | VAL | A | 682 | 12.437 | −15.382 | 28.542 | 1.00 | 10.01 | | A | C |
| ANISOU | 4308 | CB | VAL | A | 682 | 1269 | 1253 | 1281 | −24 | −8 | 3 | A | C |
| ATOM | 4310 | CG1 | VAL | A | 682 | 13.696 | −14.604 | 28.873 | 1.00 | 11.04 | | A | C |
| ANISOU | 4310 | CG1 | VAL | A | 682 | 1344 | 1480 | 1370 | −33 | −12 | −103 | A | C |
| ATOM | 4314 | CG2 | VAL | A | 682 | 12.695 | −16.309 | 27.344 | 1.00 | 11.13 | | A | C |
| ANISOU | 4314 | CG2 | VAL | A | 682 | 1483 | 1482 | 1263 | −9 | −18 | −15 | A | C |
| ATOM | 4318 | C | VAL | A | 682 | 11.887 | −15.289 | 31.013 | 1.00 | 8.96 | | A | C |
| ANISOU | 4318 | C | VAL | A | 682 | 1166 | 1175 | 1062 | −9 | −45 | 22 | A | C |
| ATOM | 4319 | O | VAL | A | 682 | 12.817 | −15.287 | 31.821 | 1.00 | 9.28 | | A | O |
| ANISOU | 4319 | O | VAL | A | 682 | 1190 | 1198 | 1138 | −105 | −21 | 12 | A | O |
| ATOM | 4320 | N | TYR | A | 683 | 10.816 | −14.515 | 31.149 | 1.00 | 7.42 | | A | N |
| ANISOU | 4320 | N | TYR | A | 683 | 1047 | 942 | 829 | −59 | −20 | −35 | A | N |
| ATOM | 4322 | CA | TYR | A | 683 | 10.624 | −13.631 | 32.288 | 1.00 | 7.98 | | A | C |
| ANISOU | 4322 | CA | TYR | A | 683 | 1074 | 1004 | 953 | −55 | 4 | −19 | A | C |
| ATOM | 4324 | CB | TYR | A | 683 | 9.360 | −12.795 | 32.077 | 1.00 | 8.15 | | A | C |
| ANISOU | 4324 | CB | TYR | A | 683 | 1027 | 998 | 1070 | −74 | 62 | −36 | A | C |
| ATOM | 4327 | CG | TYR | A | 683 | 9.083 | −11.763 | 33.155 | 1.00 | 8.77 | | A | C |
| ANISOU | 4327 | CG | TYR | A | 683 | 1163 | 1096 | 1073 | −18 | −41 | −62 | A | C |
| ATOM | 4328 | CD1 | TYR | A | 683 | 10.043 | −10.838 | 33.534 | 1.00 | 10.84 | | A | C |
| ANISOU | 4328 | CD1 | TYR | A | 683 | 1297 | 1445 | 1375 | −80 | 0 | −85 | A | C |
| ATOM | 4330 | CE1 | TYR | A | 683 | 9.781 | −9.884 | 34.499 | 1.00 | 11.06 | | A | C |
| ANISOU | 4330 | CE1 | TYR | A | 683 | 1495 | 1123 | 1581 | −12 | 3 | −24 | A | C |
| ATOM | 4332 | CZ | TYR | A | 683 | 8.562 | −9.838 | 35.089 | 1.00 | 12.24 | | A | C |
| ANISOU | 4332 | CZ | TYR | A | 683 | 1582 | 1372 | 1696 | −55 | 14 | −74 | A | C |
| ATOM | 4333 | OH | TYR | A | 683 | 8.330 | −8.897 | 36.057 | 1.00 | 15.28 | | A | O |
| ANISOU | 4333 | OH | TYR | A | 683 | 2259 | 1697 | 1848 | 56 | 92 | −190 | A | O |
| ATOM | 4335 | CE2 | TYR | A | 683 | 7.570 | −10.747 | 34.721 | 1.00 | 13.43 | | A | C |
| ANISOU | 4335 | CE2 | TYR | A | 683 | 1604 | 1727 | 1768 | −91 | −83 | −101 | A | C |
| ATOM | 4337 | CD2 | TYR | A | 683 | 7.847 | −11.693 | 33.763 | 1.00 | 11.54 | | A | C |
| ANISOU | 4337 | CD2 | TYR | A | 683 | 1252 | 1469 | 1663 | −93 | −2 | −18 | A | C |
| ATOM | 4339 | C | TYR | A | 683 | 10.565 | −14.430 | 33.586 | 1.00 | 8.07 | | A | C |
| ANISOU | 4339 | C | TYR | A | 683 | 1051 | 1069 | 946 | −108 | −28 | −44 | A | C |
| ATOM | 4340 | O | TYR | A | 683 | 11.231 | −14.062 | 34.562 | 1.00 | 7.95 | | A | O |
| ANISOU | 4340 | O | TYR | A | 683 | 1017 | 1108 | 894 | −139 | −233 | −87 | A | O |
| ATOM | 4341 | N | GLN | A | 684 | 9.791 | −15.521 | 33.615 | 1.00 | 8.72 | | A | N |
| ANISOU | 4341 | N | GLN | A | 684 | 1095 | 1129 | 1086 | −82 | −76 | 7 | A | N |
| ATOM | 4343 | CA | GLN | A | 684 | 9.722 | −16.363 | 34.802 | 1.00 | 9.04 | | A | C |
| ANISOU | 4343 | CA | GLN | A | 684 | 1145 | 1183 | 1106 | −53 | −16 | −3 | A | C |
| ATOM | 4345 | CB | GLN | A | 684 | 8.733 | −17.495 | 34.595 | 1.00 | 9.16 | | A | C |
| ANISOU | 4345 | CB | GLN | A | 684 | 1091 | 1195 | 1194 | −26 | −74 | −3 | A | C |
| ATOM | 4348 | CG | GLN | A | 684 | 8.473 | −18.335 | 35.852 | 1.00 | 10.73 | | A | C |
| ANISOU | 4348 | CG | GLN | A | 684 | 1381 | 1285 | 1408 | −113 | −11 | 38 | A | C |
| ATOM | 4351 | CD | GLN | A | 684 | 7.893 | −17.496 | 36.997 | 1.00 | 12.42 | | A | C |
| ANISOU | 4351 | CD | GLN | A | 684 | 1633 | 1631 | 1454 | 28 | 7 | 39 | A | C |
| ATOM | 4352 | OE1 | GLN | A | 684 | 6.830 | −16.884 | 36.841 | 1.00 | 16.82 | | A | O |
| ANISOU | 4352 | OE1 | GLN | A | 684 | 1974 | 2086 | 2329 | 187 | 24 | −137 | A | O |
| ATOM | 4353 | NE2 | GLN | A | 684 | 8.589 | −17.457 | 38.133 | 1.00 | 14.35 | | A | N |
| ANISOU | 4353 | NE2 | GLN | A | 684 | 1765 | 1983 | 1705 | −164 | −62 | −2 | A | N |
| ATOM | 4356 | C | GLN | A | 684 | 11.082 | −16.929 | 35.138 | 1.00 | 8.57 | | A | C |
| ANISOU | 4356 | C | GLN | A | 684 | 1118 | 1150 | 987 | −85 | −69 | 23 | A | C |
| ATOM | 4357 | O | GLN | A | 684 | 11.467 | −16.964 | 36.312 | 1.00 | 8.94 | | A | O |
| ANISOU | 4357 | O | GLN | A | 684 | 1183 | 1310 | 901 | −105 | −146 | 57 | A | O |
| ATOM | 4358 | N | MET | A | 685 | 11.819 | −17.380 | 34.131 | 1.00 | 10.03 | | A | N |
| ANISOU | 4358 | N | MET | A | 685 | 1309 | 1375 | 1126 | −90 | −26 | 19 | A | N |
| ATOM | 4360 | CA | MET | A | 685 | 13.137 | −17.948 | 34.341 | 1.00 | 11.25 | | A | C |
| ANISOU | 4360 | CA | MET | A | 685 | 1465 | 1446 | 1360 | 23 | −7 | 65 | A | C |
| ATOM | 4362 | CB | MET | A | 685 | 13.759 | −18.527 | 33.043 | 1.00 | 13.40 | | A | C |
| ANISOU | 4362 | CB | MET | A | 685 | 1718 | 1744 | 1630 | 41 | 31 | −37 | A | C |
| ATOM | 4365 | CG | MET | A | 685 | 13.060 | −19.717 | 32.372 | 1.00 | 19.86 | | A | C |
| ANISOU | 4365 | CG | MET | A | 685 | 2392 | 2405 | 2746 | −124 | −105 | −80 | A | C |
| ATOM | 4368 | SD | MET | A | 685 | 13.569 | −19.901 | 30.581 | 1.00 | 29.53 | | A | S |
| ANISOU | 4368 | SD | MET | A | 685 | 3774 | 3826 | 3618 | −102 | 214 | −258 | A | S |
| ATOM | 4369 | CE | MET | A | 685 | 15.286 | −20.484 | 30.764 | 1.00 | 30.13 | | A | C |
| ANISOU | 4369 | CE | MET | A | 685 | 3856 | 3827 | 3763 | −20 | −5 | −3 | A | C |
| ATOM | 4373 | C | MET | A | 685 | 14.078 | −16.898 | 34.926 | 1.00 | 11.10 | | A | C |
| ANISOU | 4373 | C | MET | A | 685 | 1418 | 1473 | 1327 | 37 | −22 | 64 | A | C |
| ATOM | 4374 | O | MET | A | 685 | 14.843 | −17.202 | 35.835 | 1.00 | 11.04 | | A | O |
| ANISOU | 4374 | O | MET | A | 685 | 1410 | 1534 | 1248 | 44 | −105 | 128 | A | O |
| ATOM | 4375 | N | GLU | A | 686 | 14.019 | −15.652 | 34.454 | 1.00 | 10.54 | | A | N |
| ANISOU | 4375 | N | GLU | A | 686 | 1324 | 1377 | 1302 | 67 | −66 | 69 | A | N |
| ATOM | 4377 | CA | GLU | A | 686 | 14.902 | −14.607 | 34.994 | 1.00 | 11.48 | | A | C |
| ANISOU | 4377 | CA | GLU | A | 686 | 1494 | 1451 | 1415 | 17 | −8 | 27 | A | C |
| ATOM | 4379 | CB | GLU | A | 686 | 14.850 | −13.339 | 34.134 | 1.00 | 12.27 | | A | C |
| ANISOU | 4379 | CB | GLU | A | 686 | 1590 | 1516 | 1552 | 56 | −95 | 23 | A | C |
| ATOM | 4382 | CG | GLU | A | 686 | 15.583 | −13.464 | 32.816 | 1.00 | 15.45 | | A | C |
| ANISOU | 4382 | CG | GLU | A | 686 | 1974 | 2012 | 1882 | −45 | 28 | 78 | A | C |
| ATOM | 4385 | CD | GLU | A | 686 | 17.084 | −13.665 | 32.979 | 1.00 | 17.34 | | A | C |
| ANISOU | 4385 | CD | GLU | A | 686 | 2104 | 2299 | 2185 | 23 | −36 | 152 | A | C |
| ATOM | 4386 | OE1 | GLU | A | 686 | 17.705 | −12.990 | 33.824 | 1.00 | 20.51 | | A | O |
| ANISOU | 4386 | OE1 | GLU | A | 686 | 2696 | 2619 | 2476 | −130 | 23 | 65 | A | O |
| ATOM | 4387 | OE2 | GLU | A | 686 | 17.639 | −14.500 | 32.245 | 1.00 | 22.70 | | A | O |
| ANISOU | 4387 | OE2 | GLU | A | 686 | 2785 | 3090 | 2748 | 69 | 102 | 19 | A | O |
| ATOM | 4388 | C | GLU | A | 686 | 14.527 | −14.269 | 36.433 | 1.00 | 11.50 | | A | C |
| ANISOU | 4388 | C | GLU | A | 686 | 1464 | 1491 | 1411 | −1 | −79 | −46 | A | C |
| ATOM | 4389 | O | GLU | A | 686 | 15.391 | −13.952 | 37.250 | 1.00 | 12.04 | | A | O |
| ANISOU | 4389 | O | GLU | A | 686 | 1433 | 1693 | 1447 | −23 | −188 | −23 | A | O |
| ATOM | 4390 | N | LYS | A | 687 | 13.243 | −14.314 | 36.756 | 1.00 | 11.63 | | A | N |
| ANISOU | 4390 | N | LYS | A | 687 | 1433 | 1604 | 1382 | −14 | −102 | −46 | A | N |
| ATOM | 4392 | CA | LYS | A | 687 | 12.819 | −14.108 | 38.137 | 1.00 | 12.65 | | A | C |
| ANISOU | 4392 | CA | LYS | A | 687 | 1588 | 1684 | 1532 | −16 | −11 | −87 | A | C |
| ATOM | 4394 | CB | LYS | A | 687 | 11.299 | −13.913 | 38.256 | 1.00 | 13.23 | | A | C |
| ANISOU | 4394 | CB | LYS | A | 687 | 1647 | 1753 | 1626 | −37 | −48 | −64 | A | C |
| ATOM | 4397 | CG | LYS | A | 687 | 10.854 | −12.557 | 37.748 | 1.00 | 14.35 | | A | C |
| ANISOU | 4397 | CG | LYS | A | 687 | 1812 | 1849 | 1788 | −32 | −36 | −65 | A | C |
| ATOM | 4400 | CD | LYS | A | 687 | 9.432 | −12.225 | 38.088 | 1.00 | 16.85 | | A | C |
| ANISOU | 4400 | CD | LYS | A | 687 | 2055 | 2179 | 2166 | 74 | 45 | −58 | A | C |
| ATOM | 4403 | CE | LYS | A | 687 | 8.451 | −13.068 | 37.340 | 1.00 | 18.34 | | A | C |
| ANISOU | 4403 | CE | LYS | A | 687 | 2257 | 2306 | 2402 | 7 | 22 | −106 | A | C |
| ATOM | 4406 | NZ | LYS | A | 687 | 7.056 | −12.575 | 37.590 | 1.00 | 19.25 | | A | N |
| ANISOU | 4406 | NZ | LYS | A | 687 | 2234 | 2532 | 2545 | 55 | −32 | −135 | A | N |
| ATOM | 4410 | C | LYS | A | 687 | 13.299 | −15.268 | 39.010 | 1.00 | 12.83 | | A | C |
| ANISOU | 4410 | C | LYS | A | 687 | 1596 | 1764 | 1513 | −21 | −27 | −23 | A | C |
| ATOM | 4411 | O | LYS | A | 687 | 13.721 | −15.046 | 40.150 | 1.00 | 14.48 | | A | O |
| ANISOU | 4411 | O | LYS | A | 687 | 1839 | 2049 | 1612 | −1 | −81 | −106 | A | O |
| ATOM | 4412 | N | ASP | A | 688 | 13.308 | −16.487 | 38.469 | 1.00 | 12.84 | | A | N |
| ANISOU | 4412 | N | ASP | A | 688 | 1627 | 1728 | 1522 | −18 | 0 | 61 | A | N |
| ATOM | 4414 | CA | ASP | A | 688 | 13.702 | −17.672 | 39.237 | 1.00 | 14.56 | | A | C |
| ANISOU | 4414 | CA | ASP | A | 688 | 1861 | 1897 | 1773 | 10 | −17 | 53 | A | C |
| ATOM | 4416 | CB | ASP | A | 688 | 13.302 | −18.968 | 38.521 | 1.00 | 14.83 | | A | C |
| ANISOU | 4416 | CB | ASP | A | 688 | 1929 | 1901 | 1803 | 4 | −26 | 56 | A | C |
| ATOM | 4419 | CG | ASP | A | 688 | 11.814 | −19.206 | 38.505 | 1.00 | 16.42 | | A | C |
| ANISOU | 4419 | CG | ASP | A | 688 | 2103 | 2140 | 1993 | −52 | 0 | 78 | A | C |
| ATOM | 4420 | OD1 | ASP | A | 688 | 11.063 | −18.538 | 39.240 | 1.00 | 17.23 | | A | O |
| ANISOU | 4420 | OD1 | ASP | A | 688 | 2223 | 2208 | 2113 | −64 | 77 | 112 | A | O |
| ATOM | 4421 | OD2 | ASP | A | 688 | 11.307 | −20.066 | 37.750 | 1.00 | 19.27 | | A | O |
| ANISOU | 4421 | OD2 | ASP | A | 688 | 2544 | 2282 | 2494 | −266 | 58 | −12 | A | O |
| ATOM | 4422 | C | ASP | A | 688 | 15.203 | −17.739 | 39.502 | 1.00 | 15.66 | | A | C |
| ANISOU | 4422 | C | ASP | A | 688 | 1969 | 2053 | 1925 | 68 | −73 | 52 | A | C |
| ATOM | 4423 | O | ASP | A | 688 | 15.593 | −18.395 | 40.463 | 1.00 | 15.79 | | A | O |
| ANISOU | 4423 | O | ASP | A | 688 | 1997 | 2210 | 1792 | 46 | −105 | 206 | A | O |
| ATOM | 4424 | N | ILE | A | 689 | 16.032 | −17.093 | 38.677 | 1.00 | 18.10 | | A | N |
| ANISOU | 4424 | N | ILE | A | 689 | 2301 | 2353 | 2221 | 12 | −20 | 53 | A | N |
| ATOM | 4426 | CA | ILE | A | 689 | 17.498 | −17.136 | 38.821 | 1.00 | 20.93 | | A | C |
| ANISOU | 4426 | CA | ILE | A | 689 | 2581 | 2724 | 2644 | 21 | −41 | 11 | A | C |
| ATOM | 4428 | CB | ILE | A | 689 | 18.220 | −17.290 | 37.453 | 1.00 | 21.72 | | A | C |
| ANISOU | 4428 | CB | ILE | A | 689 | 2673 | 2784 | 2795 | −23 | 18 | −28 | A | C |
| ATOM | 4430 | CG1 | ILE | A | 689 | 18.089 | −16.038 | 36.614 | 1.00 | 21.57 | | A | C |
| ANISOU | 4430 | CG1 | ILE | A | 689 | 2669 | 2823 | 2700 | 17 | −34 | −36 | A | C |
| ATOM | 4433 | CD1 | ILE | A | 689 | 19.026 | −15.997 | 35.454 | 1.00 | 22.85 | | A | C |
| ANISOU | 4433 | CD1 | ILE | A | 689 | 2820 | 2972 | 2889 | −4 | 60 | −13 | A | C |
| ATOM | 4437 | CG2 | ILE | A | 689 | 17.710 | −18.490 | 36.694 | 1.00 | 22.88 | | A | C |
| ANISOU | 4437 | CG2 | ILE | A | 689 | 2908 | 2946 | 2839 | −11 | 8 | −91 | A | C |
| ATOM | 4441 | C | ILE | A | 689 | 18.087 | −15.913 | 39.530 | 1.00 | 23.30 | | A | C |
| ANISOU | 4441 | C | ILE | A | 689 | 2912 | 2957 | 2984 | −36 | −22 | −17 | A | C |
| ATOM | 4442 | O | ILE | A | 689 | 19.277 | −15.920 | 39.902 | 1.00 | 23.81 | | A | O |
| ANISOU | 4442 | O | ILE | A | 689 | 2927 | 3059 | 3058 | −22 | −29 | 37 | A | O |
| ATOM | 4443 | N | ALA | A | 690 | 17.262 | −14.881 | 39.710 | 1.00 | 25.55 | | A | N |
| ANISOU | 4443 | N | ALA | A | 690 | 3207 | 3235 | 3264 | 20 | −2 | 20 | A | N |
| ATOM | 4445 | CA | ALA | A | 690 | 17.618 | −13.679 | 40.471 | 1.00 | 27.12 | | A | C |
| ANISOU | 4445 | CA | ALA | A | 690 | 3435 | 3420 | 3448 | −18 | −3 | −27 | A | C |
| ATOM | 4447 | CB | ALA | A | 690 | 16.581 | −12.595 | 40.243 | 1.00 | 27.19 | | A | C |
| ANISOU | 4447 | CB | ALA | A | 690 | 3445 | 3474 | 3410 | 1 | 3 | 6 | A | C |
| ATOM | 4451 | C | ALA | A | 690 | 17.747 | −13.963 | 41.968 | 1.00 | 28.89 | | A | C |
| ANISOU | 4451 | C | ALA | A | 690 | 3685 | 3655 | 3634 | −5 | 5 | −10 | A | C |
| ATOM | 4452 | O | ALA | A | 690 | 16.742 | −14.025 | 42.694 | 1.00 | 28.87 | | A | O |
| ANISOU | 4452 | O | ALA | A | 690 | 3666 | 3645 | 3656 | −33 | −33 | −25 | A | O |
| ATOM | 4453 | N | MET | A | 691 | 18.996 | −14.135 | 42.405 | 1.00 | 30.85 | | A | N |
| ANISOU | 4453 | N | MET | A | 691 | 3873 | 3933 | 3914 | −5 | −14 | −11 | A | N |
| ATOM | 4455 | CA | MET | A | 691 | 19.370 | −14.457 | 43.786 | 1.00 | 32.32 | | A | C |
| ANISOU | 4455 | CA | MET | A | 691 | 4087 | 4098 | 4091 | 1 | −19 | 27 | A | C |
| ATOM | 4457 | CB | MET | A | 691 | 18.774 | −13.495 | 44.826 | 1.00 | 33.00 | | A | C |
| ANISOU | 4457 | CB | MET | A | 691 | 4151 | 4222 | 4164 | −11 | 3 | −11 | A | C |
| ATOM | 4460 | CG | MET | A | 691 | 18.728 | −12.016 | 44.416 | 1.00 | 35.89 | | A | C |
| ANISOU | 4460 | CG | MET | A | 691 | 4550 | 4476 | 4609 | −31 | −30 | 84 | A | C |
| ATOM | 4463 | SD | MET | A | 691 | 18.072 | −10.919 | 45.716 | 1.00 | 40.73 | | A | S |
| ANISOU | 4463 | SD | MET | A | 691 | 5194 | 5075 | 5207 | 38 | 82 | −71 | A | S |
| ATOM | 4464 | CE | MET | A | 691 | 17.146 | −9.766 | 44.717 | 1.00 | 40.93 | | A | C |
| ANISOU | 4464 | CE | MET | A | 691 | 5182 | 5149 | 5218 | 11 | 18 | 19 | A | C |
| ATOM | 4468 | C | MET | A | 691 | 18.924 | −15.883 | 44.029 | 1.00 | 32.38 | | A | C |
| ANISOU | 4468 | C | MET | A | 691 | 4093 | 4096 | 4113 | −9 | −3 | 42 | A | C |
| ATOM | 4469 | O | MET | A | 691 | 19.736 | −16.802 | 44.132 | 1.00 | 33.00 | | A | O |
| ANISOU | 4469 | O | MET | A | 691 | 4199 | 4106 | 4234 | −3 | 58 | 28 | A | O |
| ATOM | 4470 | OXT | MET | A | 691 | 17.733 | −16.130 | 44.091 | 1.00 | 32.92 | | A | O |
| ANISOU | 4470 | OXT | MET | A | 691 | 4172 | 4147 | 4189 | 3 | −42 | 56 | A | O |
| ATOM | 4471 | AS | AS | C | 459 | −3.095 | 18.704 | 14.731 | 0.50 | 33.56 | | C | AS |
| ATOM | 4472 | AS | AS | C | 677 | 1.446 | −19.607 | 27.573 | 0.50 | 35.18 | | C | AS |
| ATOM | 4473 | O | HOH | W | 1 | 19.479 | −9.528 | 13.063 | 1.00 | 13.87 | | W | O |
| ATOM | 4476 | O | HOH | W | 2 | 16.961 | −5.517 | 10.992 | 1.00 | 15.15 | | W | O |
| ATOM | 4479 | O | HOH | W | 3 | 6.300 | −4.344 | 9.624 | 1.00 | 15.98 | | W | O |
| ATOM | 4482 | O | HOH | W | 4 | 24.481 | −5.000 | 22.089 | 1.00 | 17.95 | | W | O |
| ATOM | 4485 | O | HOH | W | 5 | 14.777 | 3.240 | 18.099 | 1.00 | 15.35 | | W | O |
| ATOM | 4488 | O | HOH | W | 6 | 23.760 | −5.968 | 26.879 | 1.00 | 16.90 | | W | O |
| ATOM | 4491 | O | HOH | W | 7 | 1.042 | −6.829 | 13.337 | 1.00 | 18.66 | | W | O |
| ATOM | 4494 | O | HOH | W | 8 | 19.751 | −2.578 | 2.644 | 1.00 | 20.13 | | W | O |
| ATOM | 4497 | O | HOH | W | 9 | 17.092 | −4.813 | 8.543 | 1.00 | 15.12 | | W | O |
| ATOM | 4500 | O | HOH | W | 10 | −4.221 | 2.343 | 24.689 | 1.00 | 19.64 | | W | O |
| ATOM | 4503 | O | HOH | W | 11 | 18.305 | 2.556 | 18.877 | 1.00 | 18.58 | | W | O |
| ATOM | 4506 | O | HOH | W | 12 | −7.953 | −9.648 | 20.392 | 1.00 | 17.80 | | W | O |
| ATOM | 4509 | O | HOH | W | 13 | 17.994 | 2.499 | 9.123 | 1.00 | 18.39 | | W | O |
| ATOM | 4512 | O | HOH | W | 14 | 14.790 | 6.896 | 22.565 | 1.00 | 16.75 | | W | O |
| ATOM | 4515 | O | HOH | W | 15 | 27.168 | −8.111 | 21.389 | 1.00 | 17.65 | | W | O |
| ATOM | 4518 | O | HOH | W | 16 | −0.333 | −9.332 | 7.954 | 1.00 | 21.84 | | W | O |
| ATOM | 4521 | O | HOH | W | 17 | −2.677 | 18.484 | −2.169 | 1.00 | 21.93 | | W | O |
| ATOM | 4524 | O | HOH | W | 18 | 15.551 | −2.808 | 37.019 | 1.00 | 18.87 | | W | O |
| ATOM | 4527 | O | HOH | W | 19 | 4.454 | −2.679 | −1.326 | 1.00 | 19.19 | | W | O |
| ATOM | 4530 | O | HOH | W | 20 | 10.008 | −5.335 | 9.247 | 1.00 | 23.00 | | W | O |
| ATOM | 4533 | O | HOH | W | 21 | 25.415 | −5.343 | 24.575 | 1.00 | 21.32 | | W | O |
| ATOM | 4536 | O | HOH | W | 22 | 13.461 | 9.250 | 27.873 | 1.00 | 18.51 | | W | O |
| ATOM | 4539 | O | HOH | W | 23 | 26.898 | −11.622 | 23.911 | 1.00 | 23.24 | | W | O |
| ATOM | 4542 | O | HOH | W | 24 | 18.133 | −15.564 | 12.221 | 1.00 | 21.74 | | W | O |
| ATOM | 4545 | O | HOH | W | 25 | 12.105 | −12.266 | 3.348 | 1.00 | 17.80 | | W | O |
| ATOM | 4548 | O | HOH | W | 26 | −0.257 | −15.874 | 24.048 | 1.00 | 23.98 | | W | O |
| ATOM | 4551 | O | HOH | W | 27 | 13.196 | 0.899 | 35.037 | 1.00 | 21.23 | | W | O |
| ATOM | 4554 | O | HOH | W | 28 | 0.840 | −21.860 | 18.764 | 1.00 | 21.06 | | W | O |
| ATOM | 4557 | O | HOH | W | 29 | 17.583 | −9.596 | 36.668 | 1.00 | 19.88 | | W | O |
| ATOM | 4560 | O | HOH | W | 30 | −2.774 | −12.358 | 18.133 | 1.00 | 19.66 | | W | O |
| ATOM | 4563 | O | HOH | W | 31 | 4.584 | −14.069 | 12.249 | 1.00 | 21.22 | | W | O |
| ATOM | 4566 | O | HOH | W | 32 | 23.562 | −4.487 | 0.477 | 1.00 | 22.27 | | W | O |
| ATOM | 4569 | O | HOH | W | 33 | 26.010 | −13.571 | 18.980 | 1.00 | 22.84 | | W | O |
| ATOM | 4572 | O | HOH | W | 34 | 9.525 | −6.939 | 37.377 | 1.00 | 27.84 | | W | O |
| ATOM | 4575 | O | HOH | W | 35 | −2.681 | 2.630 | 27.804 | 1.00 | 21.91 | | W | O |
| ATOM | 4578 | O | HOH | W | 36 | 4.342 | 12.092 | 26.768 | 1.00 | 24.91 | | W | O |
| ATOM | 4581 | O | HOH | W | 37 | 7.984 | −18.825 | 20.209 | 1.00 | 25.15 | | W | O |
| ATOM | 4584 | O | HOH | W | 38 | 18.468 | 4.977 | 22.255 | 1.00 | 22.38 | | W | O |
| ATOM | 4587 | O | HOH | W | 39 | 14.159 | −19.657 | 23.215 | 1.00 | 21.41 | | W | O |
| ATOM | 4590 | O | HOH | W | 40 | 0.586 | −0.637 | 8.269 | 1.00 | 24.36 | | W | O |
| ATOM | 4593 | O | HOH | W | 41 | 2.295 | 11.344 | 25.221 | 1.00 | 27.87 | | W | O |
| ATOM | 4596 | O | HOH | W | 42 | −7.451 | 21.442 | 33.821 | 1.00 | 27.32 | | W | O |
| ATOM | 4599 | O | HOH | W | 43 | −3.039 | 25.687 | 20.469 | 1.00 | 24.75 | | W | O |
| ATOM | 4602 | O | HOH | W | 44 | 3.160 | −23.086 | 16.130 | 1.00 | 27.74 | | W | O |
| ATOM | 4605 | O | HOH | W | 45 | 17.028 | −17.456 | 7.945 | 1.00 | 23.83 | | W | O |
| ATOM | 4608 | O | HOH | W | 46 | −4.639 | 26.435 | 28.046 | 1.00 | 31.05 | | W | O |
| ATOM | 4611 | O | HOH | W | 47 | −10.923 | −5.627 | 20.143 | 1.00 | 22.57 | | W | O |
| ATOM | 4614 | O | HOH | W | 48 | 19.351 | −18.606 | 28.704 | 1.00 | 28.51 | | W | O |
| ATOM | 4617 | O | HOH | W | 49 | 21.847 | −11.720 | 31.609 | 1.00 | 26.04 | | W | O |
| ATOM | 4620 | O | HOH | W | 50 | 10.662 | −4.033 | −5.247 | 1.00 | 31.05 | | W | O |
| ATOM | 4623 | O | HOH | W | 51 | 20.457 | −8.195 | 36.064 | 1.00 | 27.03 | | W | O |
| ATOM | 4626 | O | HOH | W | 52 | −3.376 | 22.364 | 15.888 | 1.00 | 25.70 | | W | O |
| ATOM | 4629 | O | HOH | W | 53 | −2.265 | −15.175 | 15.979 | 1.00 | 29.94 | | W | O |
| ATOM | 4632 | O | HOH | W | 54 | −2.279 | −8.980 | 24.279 | 1.00 | 26.59 | | W | O |
| ATOM | 4635 | O | HOH | W | 55 | 11.181 | 3.524 | −1.057 | 1.00 | 22.83 | | W | O |
| ATOM | 4638 | O | HOH | W | 56 | 17.643 | −12.520 | 36.367 | 1.00 | 27.57 | | W | O |
| ATOM | 4641 | O | HOH | W | 57 | 1.623 | 1.370 | 10.079 | 1.00 | 25.21 | | W | O |
| ATOM | 4644 | O | HOH | W | 58 | 15.664 | −13.804 | 45.226 | 1.00 | 34.15 | | W | O |
| ATOM | 4647 | O | HOH | W | 59 | 6.256 | −9.797 | 37.610 | 1.00 | 34.43 | | W | O |
| ATOM | 4650 | O | HOH | W | 60 | 25.815 | −12.306 | 14.831 | 1.00 | 27.33 | | W | O |
| ATOM | 4653 | O | HOH | W | 61 | 5.824 | −14.786 | 33.356 | 1.00 | 25.11 | | W | O |
| ATOM | 4656 | O | HOH | W | 62 | 16.873 | −4.248 | −1.845 | 1.00 | 32.13 | | W | O |
| ATOM | 4659 | O | HOH | W | 63 | 5.848 | −20.107 | 12.441 | 1.00 | 26.50 | | W | O |
| ATOM | 4662 | O | HOH | W | 64 | 14.032 | 24.397 | 19.568 | 1.00 | 36.45 | | W | O |
| ATOM | 4665 | O | HOH | W | 65 | 12.803 | −10.837 | 0.729 | 1.00 | 28.65 | | W | O |
| ATOM | 4668 | O | HOH | W | 66 | 13.165 | 9.672 | 0.079 | 1.00 | 33.59 | | W | O |
| ATOM | 4671 | O | HOH | W | 67 | 16.453 | −17.500 | 33.129 | 1.00 | 24.48 | | W | O |
| ATOM | 4674 | O | HOH | W | 68 | 2.439 | 17.984 | 17.888 | 1.00 | 22.03 | | W | O |
| ATOM | 4677 | O | HOH | W | 69 | 11.257 | −4.973 | 36.931 | 1.00 | 28.56 | | W | O |
| ATOM | 4680 | O | HOH | W | 70 | 11.119 | 4.907 | 15.842 | 1.00 | 35.29 | | W | O |
| ATOM | 4683 | O | HOH | W | 71 | 6.008 | −10.532 | 5.341 | 1.00 | 24.54 | | W | O |
| ATOM | 4686 | O | HOH | W | 72 | −2.020 | 23.984 | 11.556 | 1.00 | 28.27 | | W | O |
| ATOM | 4689 | O | HOH | W | 73 | 5.919 | −14.701 | 35.857 | 1.00 | 34.04 | | W | O |
| ATOM | 4692 | O | HOH | W | 74 | 8.884 | −0.202 | 8.933 | 1.00 | 24.05 | | W | O |
| ATOM | 4695 | O | HOH | W | 75 | −9.784 | 22.893 | 33.258 | 1.00 | 34.77 | | W | O |
| ATOM | 4698 | O | HOH | W | 76 | 6.001 | 5.485 | 2.908 | 1.00 | 34.66 | | W | O |
| ATOM | 4701 | O | HOH | W | 77 | 5.311 | −21.415 | 10.117 | 1.00 | 27.03 | | W | O |
| ATOM | 4704 | O | HOH | W | 78 | 23.051 | 3.901 | 2.619 | 1.00 | 27.45 | | W | O |
| ATOM | 4707 | O | HOH | W | 79 | 8.600 | 25.383 | 26.603 | 1.00 | 34.39 | | W | O |
| ATOM | 4710 | O | HOH | W | 80 | 3.627 | −20.342 | 13.945 | 1.00 | 28.33 | | W | O |
| ATOM | 4713 | O | HOH | W | 81 | −6.693 | −5.138 | 9.294 | 1.00 | 27.84 | | W | O |
| ATOM | 4716 | O | HOH | W | 82 | 26.573 | −7.308 | 10.342 | 1.00 | 30.01 | | W | O |
| ATOM | 4719 | O | HOH | W | 83 | 18.225 | −7.928 | −2.386 | 1.00 | 29.57 | | W | O |
| ATOM | 4722 | O | HOH | W | 84 | −2.632 | −9.217 | 9.188 | 1.00 | 35.57 | | W | O |
| ATOM | 4725 | O | HOH | W | 85 | 24.388 | 5.011 | 8.985 | 1.00 | 38.75 | | W | O |
| ATOM | 4728 | O | HOH | W | 86 | −2.044 | −18.488 | 18.517 | 1.00 | 25.55 | | W | O |
| ATOM | 4731 | O | HOH | W | 87 | 20.358 | −12.721 | 33.680 | 1.00 | 34.51 | | W | O |
| ATOM | 4734 | O | HOH | W | 88 | 21.485 | −18.154 | 22.079 | 1.00 | 32.93 | | W | O |
| ATOM | 4737 | O | HOH | W | 89 | −8.489 | −10.513 | 12.287 | 1.00 | 52.27 | | W | O |
| ATOM | 4740 | O | HOH | W | 90 | −14.058 | 25.192 | 26.302 | 1.00 | 37.49 | | W | O |
| ATOM | 4743 | O | HOH | W | 91 | −8.604 | −12.364 | 15.709 | 1.00 | 27.58 | | W | O |
| ATOM | 4746 | O | HOH | W | 92 | 1.743 | −13.653 | 12.892 | 1.00 | 27.13 | | W | O |
| ATOM | 4749 | O | HOH | W | 93 | −14.328 | 5.985 | 16.063 | 1.00 | 35.97 | | W | O |
| ATOM | 4752 | O | HOH | W | 94 | 5.089 | −4.158 | 35.982 | 1.00 | 32.25 | | W | O |
| ATOM | 4755 | O | HOH | W | 95 | 2.555 | −18.427 | 6.350 | 1.00 | 46.30 | | W | O |
| ATOM | 4758 | O | HOH | W | 96 | −6.583 | 24.083 | 13.571 | 1.00 | 32.28 | | W | O |
| ATOM | 4761 | O | HOH | W | 97 | 3.120 | −6.286 | 32.922 | 1.00 | 28.76 | | W | O |
| ATOM | 4764 | O | HOH | W | 98 | 12.911 | −20.364 | 20.813 | 1.00 | 32.18 | | W | O |
| ATOM | 4767 | O | HOH | W | 99 | 9.662 | −20.723 | 23.943 | 1.00 | 28.10 | | W | O |
| ATOM | 4770 | O | HOH | W | 100 | 29.818 | 2.171 | 8.058 | 1.00 | 38.15 | | W | O |
| ATOM | 4773 | O | HOH | W | 101 | 22.923 | 4.746 | 34.609 | 1.00 | 28.23 | | W | O |
| ATOM | 4776 | O | HOH | W | 102 | 18.586 | 5.476 | 3.189 | 1.00 | 32.67 | | W | O |
| ATOM | 4779 | O | HOH | W | 103 | 23.350 | 2.836 | 26.154 | 1.00 | 29.63 | | W | O |
| ATOM | 4782 | O | HOH | W | 104 | −14.292 | 5.543 | 20.132 | 1.00 | 40.05 | | W | O |
| ATOM | 4785 | O | HOH | W | 105 | 21.006 | −1.829 | −1.022 | 1.00 | 33.79 | | W | O |
| ATOM | 4788 | O | HOH | W | 106 | 25.675 | −13.300 | 12.460 | 1.00 | 31.34 | | W | O |
| ATOM | 4791 | O | HOH | W | 107 | −14.390 | 18.999 | 19.688 | 1.00 | 40.08 | | W | O |
| ATOM | 4794 | O | HOH | W | 108 | 15.107 | −0.283 | 0.138 | 1.00 | 24.96 | | W | O |
| ATOM | 4797 | O | HOH | W | 109 | 21.210 | 4.757 | 9.005 | 1.00 | 47.23 | | W | O |
| ATOM | 4800 | O | HOH | W | 110 | 18.421 | 10.903 | 28.240 | 1.00 | 37.02 | | W | O |
| ATOM | 4803 | O | HOH | W | 111 | −6.564 | 5.275 | 23.280 | 1.00 | 40.82 | | W | O |
| ATOM | 4806 | O | HOH | W | 112 | −11.988 | 7.492 | 39.713 | 1.00 | 32.17 | | W | O |
| ATOM | 4809 | O | HOH | W | 113 | 0.944 | −3.471 | 5.721 | 1.00 | 32.52 | | W | O |
| ATOM | 4812 | O | HOH | W | 114 | 6.227 | 2.802 | 3.217 | 1.00 | 26.41 | | W | O |
| ATOM | 4815 | O | HOH | W | 115 | 3.619 | 0.859 | 39.059 | 1.00 | 56.40 | | W | O |
| ATOM | 4818 | O | HOH | W | 116 | 26.615 | −5.575 | 2.785 | 1.00 | 34.50 | | W | O |
| ATOM | 4821 | O | HOH | W | 117 | 5.391 | 6.091 | 14.350 | 1.00 | 28.96 | | W | O |
| ATOM | 4824 | O | HOH | W | 118 | 8.361 | 18.917 | 31.821 | 1.00 | 54.83 | | W | O |
| ATOM | 4827 | O | HOH | W | 119 | 27.592 | −5.870 | 29.687 | 1.00 | 34.00 | | W | O |
| ATOM | 4830 | O | HOH | W | 120 | 26.393 | −9.066 | 12.033 | 1.00 | 34.61 | | W | O |
| ATOM | 4833 | O | HOH | W | 121 | 24.039 | 4.111 | 28.493 | 1.00 | 49.57 | | W | O |
| ATOM | 4836 | O | HOH | W | 122 | 0.385 | 10.038 | 33.013 | 1.00 | 43.66 | | W | O |
| ATOM | 4839 | O | HOH | W | 123 | −1.581 | 28.019 | 31.964 | 1.00 | 42.03 | | W | O |
| ATOM | 4842 | O | HOH | W | 124 | −0.402 | 18.593 | 1.186 | 1.00 | 33.08 | | W | O |
| ATOM | 4845 | O | HOH | W | 125 | −14.153 | 22.528 | 22.865 | 1.00 | 39.67 | | W | O |
| ATOM | 4848 | O | HOH | W | 126 | 1.422 | 19.788 | 5.361 | 1.00 | 33.75 | | W | O |
| ATOM | 4851 | O | HOH | W | 127 | 6.159 | 2.368 | 5.998 | 1.00 | 30.29 | | W | O |
| ATOM | 4854 | O | HOH | W | 128 | 21.291 | −16.934 | 9.903 | 1.00 | 45.24 | | W | O |
| ATOM | 4857 | O | HOH | W | 129 | 0.770 | −6.150 | 31.663 | 1.00 | 42.18 | | W | O |
| ATOM | 4860 | O | HOH | W | 130 | −4.043 | −12.320 | 12.617 | 1.00 | 42.29 | | W | O |
| ATOM | 4863 | O | HOH | W | 131 | 20.383 | 9.724 | 26.884 | 1.00 | 36.62 | | W | O |
| ATOM | 4866 | O | HOH | W | 132 | −2.168 | 11.721 | 35.512 | 1.00 | 40.29 | | W | O |
| ATOM | 4869 | O | HOH | W | 133 | 15.005 | 2.551 | −1.347 | 1.00 | 32.89 | | W | O |
| ATOM | 4872 | O | HOH | W | 134 | 8.794 | −21.009 | 38.199 | 1.00 | 46.17 | | W | O |
| ATOM | 4875 | O | HOH | W | 135 | 2.518 | −13.122 | 30.733 | 1.00 | 38.37 | | W | O |
| ATOM | 4878 | O | HOH | W | 136 | 7.635 | −12.706 | 1.407 | 1.00 | 29.71 | | W | O |
| ATOM | 4881 | O | HOH | W | 137 | −8.685 | 24.816 | 12.760 | 1.00 | 44.11 | | W | O |
| ATOM | 4884 | O | HOH | W | 138 | −2.636 | −3.254 | 8.677 | 1.00 | 27.21 | | W | O |
| ATOM | 4887 | O | HOH | W | 139 | 15.768 | −18.358 | 12.147 | 1.00 | 40.67 | | W | O |
| ATOM | 4890 | O | HOH | W | 140 | 10.814 | 24.683 | 22.492 | 1.00 | 32.90 | | W | O |
| ATOM | 4893 | O | HOH | W | 141 | 28.656 | −9.702 | 31.405 | 1.00 | 50.52 | | W | O |
| ATOM | 4896 | O | HOH | W | 142 | −6.161 | −0.734 | 28.566 | 1.00 | 35.85 | | W | O |
| ATOM | 4899 | O | HOH | W | 143 | 26.107 | 4.685 | 13.078 | 1.00 | 36.41 | | W | O |
| ATOM | 4902 | O | HOH | W | 144 | −14.326 | 16.032 | 30.233 | 1.00 | 40.43 | | W | O |
| ATOM | 4905 | O | HOH | W | 145 | 25.066 | −1.521 | 25.107 | 1.00 | 34.70 | | W | O |
| ATOM | 4908 | O | HOH | W | 146 | 13.051 | 11.026 | 25.453 | 1.00 | 34.11 | | W | O |
| ATOM | 4911 | O | HOH | W | 147 | 1.206 | 9.609 | 20.300 | 1.00 | 50.37 | | W | O |
| ATOM | 4914 | O | HOH | W | 148 | −8.334 | 7.205 | 5.910 | 1.00 | 50.55 | | W | O |
| ATOM | 4917 | O | HOH | W | 149 | 2.947 | 1.838 | 36.508 | 1.00 | 46.93 | | W | O |
| ATOM | 4920 | O | HOH | W | 150 | 21.572 | −18.163 | 40.127 | 1.00 | 45.84 | | W | O |
| ATOM | 4923 | O | HOH | W | 151 | 7.365 | 17.383 | 10.315 | 1.00 | 54.72 | | W | O |
| ATOM | 4926 | O | HOH | W | 152 | 0.176 | −7.017 | 4.002 | 1.00 | 44.22 | | W | O |
| ATOM | 4929 | O | HOH | W | 153 | 19.073 | 6.380 | 37.289 | 1.00 | 31.56 | | W | O |
| ATOM | 4932 | O | HOH | W | 154 | 5.202 | 16.145 | 34.778 | 1.00 | 54.68 | | W | O |
| ATOM | 4935 | O | HOH | W | 155 | 23.720 | 5.441 | 13.483 | 1.00 | 32.64 | | W | O |
| ATOM | 4938 | O | HOH | W | 156 | 16.401 | 5.555 | 7.238 | 1.00 | 36.23 | | W | O |
| ATOM | 4941 | O | HOH | W | 157 | −6.498 | 14.764 | 40.845 | 1.00 | 40.96 | | W | O |
| ATOM | 4944 | O | HOH | W | 158 | 28.600 | 0.254 | −2.197 | 1.00 | 53.63 | | W | O |
| ATOM | 4947 | O | HOH | W | 159 | −12.018 | 32.218 | 16.285 | 1.00 | 49.07 | | W | O |
| ATOM | 4950 | O | HOH | W | 160 | 0.086 | 5.373 | 33.101 | 1.00 | 51.51 | | W | O |
| ATOM | 4953 | O | HOH | W | 161 | 3.274 | 8.583 | 10.649 | 1.00 | 43.06 | | W | O |
| ATOM | 4956 | O | HOH | W | 162 | 22.605 | −15.423 | 14.524 | 1.00 | 41.77 | | W | O |
| ATOM | 4959 | O | HOH | W | 163 | −8.630 | 18.199 | 6.540 | 1.00 | 27.18 | | W | O |
| ATOM | 4962 | O | HOH | W | 164 | −8.686 | −8.527 | 8.537 | 1.00 | 37.06 | | W | O |
| ATOM | 4965 | O | HOH | W | 165 | 17.356 | 5.561 | −7.220 | 1.00 | 41.21 | | W | O |
| ATOM | 4968 | O | HOH | W | 166 | 3.075 | 32.008 | 7.880 | 1.00 | 40.35 | | W | O |
| ATOM | 4971 | O | HOH | W | 167 | 1.196 | 5.617 | 4.183 | 1.00 | 45.38 | | W | O |
| ATOM | 4974 | O | HOH | W | 168 | 17.656 | −7.868 | 12.209 | 1.00 | 15.06 | | W | O |
| ATOM | 4977 | O | HOH | W | 169 | 8.664 | −2.770 | 9.648 | 1.00 | 17.79 | | W | O |
| ATOM | 4980 | O | HOH | W | 170 | 16.832 | 4.787 | 18.630 | 1.00 | 23.56 | | W | O |
| ATOM | 4983 | O | HOH | W | 171 | 25.819 | −2.202 | 22.551 | 1.00 | 29.20 | | W | O |
| ATOM | 4986 | O | HOH | W | 172 | 16.837 | 6.486 | 20.798 | 1.00 | 27.27 | | W | O |
| ATOM | 4989 | O | HOH | W | 173 | 27.727 | −6.757 | 23.692 | 1.00 | 26.70 | | W | O |
| ATOM | 4992 | O | HOH | W | 174 | 2.136 | 34.684 | 8.355 | 1.00 | 27.14 | | W | O |
| ATOM | 4995 | O | HOH | W | 175 | 14.492 | −0.335 | 37.262 | 1.00 | 31.03 | | W | O |
| ATOM | 4998 | O | HOH | W | 176 | −3.745 | −17.152 | 16.943 | 1.00 | 33.32 | | W | O |
| ATOM | 5001 | O | HOH | W | 177 | −1.756 | −18.155 | 23.813 | 1.00 | 32.25 | | W | O |
| ATOM | 5004 | O | HOH | W | 178 | 25.489 | −4.554 | 28.614 | 1.00 | 23.71 | | W | O |
| ATOM | 5007 | O | HOH | W | 179 | −2.274 | 20.476 | −0.391 | 1.00 | 28.26 | | W | O |
| ATOM | 5010 | O | HOH | W | 180 | 15.514 | −22.083 | 23.613 | 1.00 | 31.71 | | W | O |
| ATOM | 5013 | O | HOH | W | 181 | −1.046 | −11.416 | 25.140 | 1.00 | 31.45 | | W | O |
| ATOM | 5016 | O | HOH | W | 182 | −2.632 | 28.253 | 21.086 | 1.00 | 32.74 | | W | O |
| ATOM | 5019 | O | HOH | W | 183 | 11.904 | −19.349 | 24.906 | 1.00 | 30.37 | | W | O |
| ATOM | 5022 | O | HOH | W | 184 | 32.998 | 2.770 | 8.818 | 1.00 | 46.53 | | W | O |
| ATOM | 5025 | O | HOH | W | 185 | 28.730 | −4.730 | 4.511 | 1.00 | 41.03 | | W | O |
| ATOM | 5028 | O | HOH | W | 186 | −1.306 | −13.421 | 23.428 | 1.00 | 30.60 | | W | O |
| ATOM | 5031 | O | HOH | W | 187 | −5.846 | −9.685 | 22.266 | 1.00 | 42.87 | | W | O |
| ATOM | 5034 | O | HOH | W | 188 | 18.729 | −2.113 | 0.135 | 1.00 | 31.07 | | W | O |
| ATOM | 5037 | O | HOH | W | 189 | 13.762 | 4.268 | 15.601 | 1.00 | 27.23 | | W | O |
| ATOM | 5040 | O | HOH | W | 190 | 29.119 | −10.372 | 25.129 | 1.00 | 31.33 | | W | O |
| ATOM | 5043 | O | HOH | W | 191 | 18.260 | −17.081 | 10.290 | 1.00 | 35.26 | | W | O |
| ATOM | 5046 | O | HOH | W | 192 | −1.716 | −21.180 | 18.016 | 1.00 | 32.32 | | W | O |
| ATOM | 5049 | O | HOH | W | 193 | 20.664 | −16.507 | 13.211 | 1.00 | 32.95 | | W | O |
| ATOM | 5052 | O | HOH | W | 194 | 7.956 | 2.015 | 7.876 | 1.00 | 33.80 | | W | O |
| ATOM | 5055 | O | HOH | W | 195 | 4.019 | 15.799 | 17.163 | 1.00 | 33.98 | | W | O |
| ATOM | 5058 | O | HOH | W | 196 | 10.942 | −21.613 | 4.110 | 1.00 | 40.01 | | W | O |
| ATOM | 5061 | O | HOH | W | 197 | 15.808 | 24.518 | 17.442 | 1.00 | 34.61 | | W | O |
| ATOM | 5064 | O | HOH | W | 198 | −15.812 | 12.856 | 34.586 | 1.00 | 57.34 | | W | O |
| ATOM | 5067 | O | HOH | W | 199 | 4.936 | −12.559 | 3.726 | 1.00 | 31.32 | | W | O |
| ATOM | 5070 | O | HOH | W | 200 | 15.915 | −16.350 | 31.154 | 1.00 | 40.13 | | W | O |
| ATOM | 5073 | O | HOH | W | 201 | 10.572 | −13.606 | 1.569 | 1.00 | 32.28 | | W | O |
| ATOM | 5076 | O | HOH | W | 202 | 20.085 | 7.998 | 21.383 | 1.00 | 47.13 | | W | O |
| ATOM | 5079 | O | HOH | W | 203 | −1.727 | −3.536 | 6.311 | 1.00 | 40.15 | | W | O |
| ATOM | 5082 | O | HOH | W | 204 | 28.069 | −13.639 | 22.482 | 1.00 | 38.00 | | W | O |
| ATOM | 5085 | O | HOH | W | 205 | 2.428 | 8.130 | 33.485 | 1.00 | 56.76 | | W | O |
| ATOM | 5088 | O | HOH | W | 206 | 4.112 | 7.796 | 19.654 | 1.00 | 43.60 | | W | O |
| ATOM | 5091 | O | HOH | W | 207 | −16.832 | 18.512 | 28.867 | 1.00 | 43.33 | | W | O |
| ATOM | 5094 | O | HOH | W | 208 | 4.438 | 6.672 | 36.992 | 1.00 | 49.89 | | W | O |
| ATOM | 5097 | O | HOH | W | 209 | 24.369 | −10.892 | 0.049 | 1.00 | 36.67 | | W | O |
| ATOM | 5100 | O | HOH | W | 210 | 20.567 | −12.279 | 41.840 | 1.00 | 45.28 | | W | O |
| ATOM | 5103 | O | HOH | W | 211 | 23.638 | −17.238 | 22.606 | 1.00 | 38.24 | | W | O |
| ATOM | 5106 | O | HOH | W | 212 | 22.584 | 4.986 | 6.358 | 1.00 | 40.70 | | W | O |
| ATOM | 5109 | O | HOH | W | 213 | −12.649 | 13.319 | 22.068 | 1.00 | 32.40 | | W | O |
| ATOM | 5112 | O | HOH | W | 214 | 7.869 | −4.780 | 37.501 | 1.00 | 43.91 | | W | O |
| ATOM | 5115 | O | HOH | W | 215 | 22.264 | −16.129 | 40.997 | 1.00 | 39.04 | | W | O |
| ATOM | 5118 | O | HOH | W | 216 | 7.155 | −17.336 | 2.301 | 1.00 | 39.75 | | W | O |
| ATOM | 5121 | O | HOH | W | 217 | −9.374 | 10.623 | 2.581 | 1.00 | 46.78 | | W | O |
| ATOM | 5124 | O | HOH | W | 218 | −9.826 | 6.624 | 11.499 | 1.00 | 34.26 | | W | O |
| ATOM | 5127 | O | HOH | W | 219 | −8.295 | −12.287 | 20.270 | 1.00 | 39.42 | | W | O |
| ATOM | 5130 | O | HOH | W | 220 | 22.830 | −14.126 | 30.372 | 1.00 | 46.91 | | W | O |
| ATOM | 5133 | O | HOH | W | 221 | −3.842 | 27.866 | 25.606 | 1.00 | 34.41 | | W | O |
| ATOM | 5136 | O | HOH | W | 222 | −14.473 | 7.008 | 39.365 | 1.00 | 36.88 | | W | O |
| ATOM | 5139 | O | HOH | W | 223 | −13.757 | 24.696 | 33.168 | 1.00 | 42.28 | | W | O |
| ATOM | 5142 | O | HOH | W | 224 | −5.644 | 16.691 | 42.105 | 1.00 | 41.39 | | W | O |
| ATOM | 5145 | O | HOH | W | 225 | 25.497 | 5.411 | 36.898 | 1.00 | 48.15 | | W | O |
| ATOM | 5148 | O | HOH | W | 226 | 27.283 | 5.516 | 9.317 | 1.00 | 46.59 | | W | O |
| ATOM | 5151 | O | HOH | W | 227 | 29.498 | 4.434 | 1.020 | 1.00 | 58.47 | | W | O |
| ATOM | 5154 | O | HOH | W | 228 | 3.321 | −8.425 | 31.429 | 1.00 | 43.70 | | W | O |
| ATOM | 5157 | O | HOH | W | 229 | 24.117 | −15.190 | 12.199 | 1.00 | 41.18 | | W | O |
| ATOM | 5160 | O | HOH | W | 230 | 20.124 | −12.389 | 37.708 | 1.00 | 36.53 | | W | O |
| ATOM | 5163 | O | HOH | W | 231 | −9.229 | −1.501 | 21.266 | 1.00 | 28.25 | | W | O |
| ATOM | 5166 | O | HOH | W | 232 | −15.779 | 25.297 | 28.169 | 1.00 | 44.87 | | W | O |
| ATOM | 5169 | O | HOH | W | 233 | −0.796 | −15.490 | 26.671 | 1.00 | 35.65 | | W | O |
| ATOM | 5172 | O | HOH | W | 234 | −7.531 | 25.852 | 28.118 | 1.00 | 39.03 | | W | O |
| ATOM | 5175 | O | HOH | W | 235 | 21.351 | 4.652 | 37.194 | 1.00 | 34.74 | | W | O |
| ATOM | 5178 | O | HOH | W | 236 | −10.908 | 11.891 | 7.021 | 1.00 | 50.12 | | W | O |
| ATOM | 5181 | O | HOH | W | 237 | 20.093 | −19.985 | 39.619 | 1.00 | 41.48 | | W | O |
| ATOM | 5184 | O | HOH | W | 238 | 16.913 | 1.636 | 0.158 | 1.00 | 57.66 | | W | O |
| ATOM | 5187 | O | HOH | W | 239 | 24.550 | −11.786 | 32.361 | 1.00 | 45.44 | | W | O |
| ATOM | 5190 | O | HOH | W | 240 | 6.951 | 12.068 | 26.595 | 1.00 | 46.84 | | W | O |
| ATOM | 5193 | O | HOH | W | 241 | −2.373 | 24.816 | 15.700 | 1.00 | 32.54 | | W | O |
| ATOM | 5196 | O | HOH | W | 242 | −0.321 | −13.885 | 29.001 | 1.00 | 46.53 | | W | O |
| ATOM | 5199 | O | HOH | W | 243 | 11.563 | −21.031 | 35.283 | 1.00 | 45.62 | | W | O |
| ATOM | 5202 | O | HOH | W | 244 | 18.288 | −21.396 | 23.711 | 1.00 | 35.24 | | W | O |
| ATOM | 5205 | O | HOH | W | 245 | 22.670 | −5.732 | −1.765 | 1.00 | 40.51 | | W | O |
| ATOM | 5208 | O | HOH | W | 246 | 12.161 | −20.481 | 27.378 | 1.00 | 40.97 | | W | O |
| ATOM | 5211 | O | HOH | W | 247 | 16.743 | −19.964 | 16.894 | 1.00 | 46.39 | | W | O |
| ATOM | 5214 | O | HOH | W | 248 | 19.872 | −15.776 | 32.453 | 1.00 | 41.59 | | W | O |
| ATOM | 5217 | O | HOH | W | 249 | −14.124 | 23.466 | 20.534 | 1.00 | 45.11 | | W | O |
| ATOM | 5220 | O | HOH | W | 250 | 29.513 | 0.952 | 5.819 | 1.00 | 51.70 | | W | O |
| ATOM | 5223 | O | HOH | W | 251 | 18.691 | −2.032 | 40.376 | 1.00 | 45.89 | | W | O |
| ATOM | 5226 | O | HOH | W | 252 | 10.687 | 1.887 | −7.837 | 1.00 | 42.18 | | W | O |
| ATOM | 5229 | O | HOH | W | 253 | 12.119 | −2.579 | −7.064 | 1.00 | 40.90 | | W | O |
| ATOM | 5232 | O | HOH | W | 254 | 5.119 | 26.162 | 2.725 | 1.00 | 46.48 | | W | O |
| ATOM | 5235 | O | HOH | W | 255 | 1.481 | 20.173 | 33.847 | 1.00 | 38.28 | | W | O |
| ATOM | 5238 | O | HOH | W | 256 | 10.507 | 8.862 | 0.082 | 1.00 | 42.31 | | W | O |
| ATOM | 5241 | O | HOH | W | 257 | 25.796 | 6.387 | −1.577 | 1.00 | 53.89 | | W | O |
| ATOM | 5244 | O | HOH | W | 258 | 20.185 | 1.064 | −1.864 | 1.00 | 46.15 | | W | O |
| ATOM | 5247 | O | HOH | W | 259 | −12.742 | 25.526 | 20.010 | 1.00 | 51.93 | | W | O |
| ATOM | 5250 | O | HOH | W | 260 | 21.887 | 8.253 | 14.036 | 1.00 | 62.78 | | W | O |
| ATOM | 5253 | O | HOH | W | 261 | −10.680 | 10.655 | 33.008 | 1.00 | 44.00 | | W | O |
| ATOM | 5256 | O | HOH | W | 262 | −6.574 | 8.507 | 31.016 | 1.00 | 46.18 | | W | O |
| ATOM | 5259 | O | HOH | W | 263 | 3.942 | −12.795 | 33.099 | 1.00 | 40.21 | | W | O |
| ATOM | 5262 | O | HOH | W | 264 | 1.018 | 17.787 | 33.485 | 1.00 | 38.43 | | W | O |
| ATOM | 5265 | O | HOH | W | 265 | 26.837 | 3.753 | 28.570 | 1.00 | 46.62 | | W | O |
| ATOM | 5268 | O | HOH | W | 266 | 7.159 | −23.096 | 9.113 | 1.00 | 37.42 | | W | O |
| ATOM | 5271 | O | HOH | W | 267 | 12.960 | 13.454 | 3.068 | 1.00 | 71.06 | | W | O |
| ATOM | 5274 | O | HOH | W | 268 | 22.282 | 9.259 | 28.712 | 1.00 | 43.17 | | W | O |
| ATOM | 5277 | O | HOH | W | 269 | 9.738 | 17.174 | 27.040 | 1.00 | 53.53 | | W | O |
| ATOM | 5280 | O | HOH | W | 270 | 26.067 | −5.231 | 0.124 | 1.00 | 40.20 | | W | O |
| ATOM | 5283 | O | HOH | W | 271 | 18.928 | 13.275 | 29.767 | 1.00 | 52.97 | | W | O |
| ATOM | 5286 | O | HOH | W | 272 | 10.321 | 22.586 | 14.898 | 1.00 | 67.97 | | W | O |
| ATOM | 5289 | O | HOH | W | 273 | 16.890 | −16.446 | 41.444 | 1.00 | 35.90 | | W | O |
| ATOM | 5292 | O | HOH | W | 274 | 4.249 | −9.445 | 33.791 | 1.00 | 35.65 | | W | O |
| ATOM | 5295 | O | HOH | W | 275 | 15.844 | 5.952 | 14.465 | 1.00 | 36.89 | | W | O |
| ATOM | 5298 | O | HOH | W | 276 | 13.813 | −22.678 | 27.150 | 1.00 | 43.23 | | W | O |
| ATOM | 5301 | O | HOH | W | 277 | 27.098 | −7.831 | 1.776 | 1.00 | 40.66 | | W | O |
| ATOM | 5304 | O | HOH | W | 278 | −5.906 | 22.740 | 35.697 | 1.00 | 36.67 | | W | O |
| ATOM | 5307 | O | HOH | W | 279 | −5.273 | −16.484 | 18.783 | 1.00 | 35.36 | | W | O |
| ATOM | 5310 | O | HOH | W | 280 | −11.599 | 8.596 | 10.872 | 1.00 | 40.55 | | W | O |
| ATOM | 5313 | O | HOH | W | 281 | 17.212 | 8.689 | 19.402 | 1.00 | 43.11 | | W | O |
| ATOM | 5316 | O | HOH | W | 282 | 4.804 | 27.860 | 29.110 | 1.00 | 43.16 | | W | O |
| ATOM | 5319 | O | HOH | W | 283 | 5.083 | 13.910 | 18.423 | 1.00 | 42.13 | | W | O |
| ATOM | 5322 | O | HOH | W | 284 | 11.639 | 10.829 | 18.673 | 1.00 | 47.09 | | W | O |
| ATOM | 5325 | O | HOH | W | 285 | 10.959 | 3.360 | 12.881 | 1.00 | 43.06 | | W | O |
| ATOM | 5328 | O | HOH | W | 286 | −1.180 | −6.658 | 29.927 | 1.00 | 52.21 | | W | O |
| ATOM | 5331 | O | HOH | W | 287 | −4.817 | −13.785 | 18.381 | 1.00 | 38.59 | | W | O |
| ATOM | 5334 | O | HOH | W | 288 | −2.046 | 10.931 | 32.923 | 1.00 | 44.39 | | W | O |
| ATOM | 5337 | O | HOH | W | 289 | 21.742 | 9.750 | 23.057 | 1.00 | 50.79 | | W | O |
| ATOM | 5340 | O | HOH | W | 290 | 17.595 | 6.004 | 16.313 | 1.00 | 37.61 | | W | O |
| ATOM | 5343 | O | HOH | W | 291 | 15.078 | −23.825 | 21.692 | 1.00 | 53.24 | | W | O |
| ATOM | 5346 | O | HOH | W | 292 | 10.040 | −2.679 | 36.859 | 1.00 | 38.99 | | W | O |
| ATOM | 5349 | O | HOH | W | 293 | 24.658 | −14.439 | 28.467 | 1.00 | 42.29 | | W | O |
| ATOM | 5352 | O | HOH | W | 294 | 2.481 | 9.919 | 23.093 | 1.00 | 52.30 | | W | O |
| ATOM | 5355 | O | HOH | W | 295 | −11.544 | 10.199 | 0.951 | 1.00 | 42.96 | | W | O |
| ATOM | 5358 | O | HOH | W | 296 | 0.108 | 4.674 | 1.885 | 1.00 | 70.64 | | W | O |
| ATOM | 5361 | O | HOH | W | 297 | −4.402 | −12.590 | 20.752 | 1.00 | 50.92 | | W | O |
| ATOM | 5364 | O | HOH | W | 298 | 10.146 | −23.973 | 6.683 | 1.00 | 53.06 | | W | O |
| ATOM | 5367 | O | HOH | W | 299 | 19.627 | −20.265 | 17.138 | 1.00 | 46.12 | | W | O |
| ATOM | 5370 | O | HOH | W | 300 | 8.744 | −22.460 | 11.964 | 1.00 | 54.32 | | W | O |
| ATOM | 5373 | O | HOH | W | 301 | 19.047 | −5.541 | −3.243 | 1.00 | 45.72 | | W | O |
| ATOM | 5376 | O | HOH | W | 302 | 3.891 | −3.645 | 38.106 | 1.00 | 36.00 | | W | O |
| ATOM | 5379 | O | HOH | W | 303 | −2.492 | −8.228 | 26.553 | 1.00 | 43.16 | | W | O |
| ATOM | 5382 | O | HOH | W | 304 | −1.716 | −13.273 | 11.577 | 1.00 | 49.55 | | W | O |
| ATOM | 5385 | O | HOH | W | 305 | 10.334 | −22.828 | 32.270 | 1.00 | 49.07 | | W | O |
| ATOM | 5388 | O | HOH | W | 306 | 14.641 | 10.583 | 19.949 | 1.00 | 45.35 | | W | O |
| ATOM | 5391 | O | HOH | W | 307 | 8.072 | 1.930 | −8.536 | 1.00 | 52.79 | | W | O |
| ATOM | 5394 | O | HOH | W | 308 | −9.064 | 6.600 | 24.242 | 1.00 | 40.71 | | W | O |
| ATOM | 5397 | O | HOH | W | 309 | 11.964 | −1.093 | 38.727 | 1.00 | 41.67 | | W | O |
| ATOM | 5400 | O | HOH | W | 310 | 16.996 | 4.821 | 9.888 | 1.00 | 36.87 | | W | O |
| ATOM | 5403 | O | HOH | W | 311 | 25.026 | −3.015 | 31.517 | 1.00 | 57.41 | | W | O |
| ATOM | 5406 | O | HOH | W | 312 | −2.780 | −5.682 | 7.256 | 1.00 | 46.09 | | W | O |
| ATOM | 5409 | O | HOH | W | 313 | −18.185 | 11.925 | 34.759 | 1.00 | 40.14 | | W | O |
| ATOM | 5412 | O | HOH | W | 314 | 21.325 | 5.331 | −1.232 | 1.00 | 52.23 | | W | O |
| ATOM | 5415 | O | HOH | W | 315 | 13.289 | 3.175 | 13.754 | 1.00 | 36.05 | | W | O |
| ATOM | 5418 | O | HOH | W | 316 | 20.567 | −14.512 | 15.299 | 1.00 | 28.60 | | W | O |
| ATOM | 5421 | O | HOH | W | 317 | 6.126 | −23.281 | 15.325 | 1.00 | 31.62 | | W | O |
| ATOM | 5424 | O | HOH | W | 318 | 7.810 | −24.321 | 13.708 | 1.00 | 46.04 | | W | O |
| ATOM | 5427 | O | HOH | W | 319 | 20.804 | −18.283 | 18.320 | 1.00 | 47.41 | | W | O |
| ATOM | 5430 | O | HOH | W | 320 | 22.834 | 4.861 | −3.374 | 1.00 | 52.99 | | W | O |
| ATOM | 5433 | O | HOH | W | 321 | −2.966 | 12.613 | 0.288 | 1.00 | 49.85 | | W | O |
| ATOM | 5436 | O | HOH | W | 322 | −8.736 | 1.592 | 29.674 | 1.00 | 45.84 | | W | O |
| ATOM | 5439 | O | HOH | W | 323 | 14.053 | −21.775 | 35.865 | 1.00 | 41.60 | | W | O |
| ATOM | 5442 | O | HOH | W | 324 | −2.109 | −10.636 | 11.844 | 1.00 | 34.80 | | W | O |
| ATOM | 5445 | O | HOH | W | 325 | −0.638 | 6.885 | 14.405 | 1.00 | 48.19 | | W | O |
| ATOM | 5448 | O | HOH | W | 326 | 29.870 | −5.489 | 24.392 | 1.00 | 50.31 | | W | O |
| ATOM | 5451 | O | HOH | W | 327 | 26.074 | −16.489 | 27.423 | 1.00 | 61.10 | | W | O |
| ATOM | 5454 | O | HOH | W | 328 | −11.632 | 8.051 | 24.014 | 1.00 | 56.95 | | W | O |
| ATOM | 5457 | O | HOH | W | 329 | 7.912 | −20.605 | 14.462 | 1.00 | 44.67 | | W | O |
| ATOM | 5460 | O | HOH | W | 330 | 27.064 | −13.871 | 27.167 | 1.00 | 47.91 | | W | O |
| ATOM | 5463 | O | HOH | W | 331 | −2.714 | 11.537 | −2.222 | 1.00 | 50.23 | | W | O |
| ATOM | 5466 | O | HOH | W | 332 | −8.002 | −16.209 | 18.045 | 1.00 | 34.96 | | W | O |
| ATOM | 5469 | O | HOH | W | 333 | 33.966 | −9.397 | 32.778 | 1.00 | 54.54 | | W | O |
| ATOM | 5472 | O | HOH | W | 334 | 7.173 | −25.504 | 7.225 | 1.00 | 53.06 | | W | O |
| ATOM | 5475 | O | HOH | W | 335 | 15.475 | −20.063 | 36.278 | 1.00 | 48.34 | | W | O |
| ATOM | 5478 | O | HOH | W | 336 | 6.619 | 6.422 | 38.525 | 1.00 | 43.03 | | W | O |
| ATOM | 5481 | O | HOH | W | 337 | −1.974 | 8.937 | 15.647 | 1.00 | 63.54 | | W | O |
| ATOM | 5484 | O | HOH | W | 338 | 4.790 | 7.735 | 12.579 | 1.00 | 38.37 | | W | O |
| ATOM | 5487 | O | HOH | W | 339 | −13.989 | 17.117 | 22.115 | 1.00 | 51.92 | | W | O |
| ATOM | 5490 | O | HOH | W | 340 | −10.024 | 12.553 | 9.238 | 1.00 | 42.11 | | W | O |
| ATOM | 5493 | O | HOH | W | 341 | −13.259 | 7.921 | 26.139 | 1.00 | 42.31 | | W | O |
| ATOM | 5496 | O | HOH | W | 342 | −8.537 | −13.413 | 18.217 | 1.00 | 49.82 | | W | O |
| ATOM | 5499 | O | HOH | W | 343 | −12.272 | 13.411 | 15.526 | 1.00 | 51.61 | | W | O |
| ATOM | 5502 | O | HOH | W | 344 | −3.611 | −14.130 | 22.744 | 1.00 | 45.31 | | W | O |
| ATOM | 5505 | O | HOH | W | 345 | 16.070 | −1.755 | −1.666 | 1.00 | 35.48 | | W | O |
| ATOM | 5508 | O | HOH | W | 346 | 11.461 | 20.223 | 27.293 | 1.00 | 52.83 | | W | O |
| ATOM | 5511 | O | HOH | W | 347 | 6.322 | −4.058 | 39.620 | 1.00 | 46.01 | | W | O |
| ATOM | 5514 | O | HOH | W | 348 | 12.395 | 19.996 | 20.666 | 1.00 | 48.20 | | W | O |
| ATOM | 5517 | O | HOH | W | 349 | 11.359 | 6.167 | −7.532 | 1.00 | 48.83 | | W | O |
| ATOM | 5520 | O | HOH | W | 350 | 22.764 | 0.708 | 40.869 | 1.00 | 54.11 | | W | O |
| ATOM | 5523 | O | HOH | W | 351 | −5.503 | −18.379 | 15.462 | 1.00 | 40.42 | | W | O |
| ATOM | 5526 | O | HOH | W | 352 | −0.473 | −15.085 | 12.915 | 1.00 | 68.33 | | W | O |
| ATOM | 5529 | O | HOH | W | 353 | 20.340 | −10.912 | 39.803 | 1.00 | 45.04 | | W | O |
| ATOM | 5532 | O | HOH | W | 354 | 6.550 | 16.368 | 32.041 | 1.00 | 65.13 | | W | O |
| ATOM | 5535 | O | HOH | W | 355 | −1.548 | −11.778 | 27.806 | 1.00 | 57.13 | | W | O |
| END | | | | | | | | | | | | | |

TABLE 2
REMARK Written by DEALPDB Version 1.13 (06/02)
REMARK Thu Jan 23 14:56:07 2003

| | | |
|---|---|---|
| HEADER | ---- | XX-XXX-XX xxxx |

| COMPND | --- | |
|---|---|---|
| REMARK | 3 | |
| REMARK | 3 | REFINEMENT. |

| | | | |
|---|---|---|---|
| REMARK | 3 | PROGRAM | : REFMAC 5.1.25 |
| REMARK | 3 | AUTHORS | : MURSHUDOV, VAGIN, DODSON |
| REMARK | 3 | | |

| | | |
|---|---|---|
| REMARK | 3 | REFINEMENT TARGET : MAXIMUM LIKELIHOOD |
| REMARK | 3 | |

| | | |
|---|---|---|
| REMARK | 3 | DATA USED IN REFINEMENT. |

| | | | | | |
|---|---|---|---|---|---|
| REMARK | 3 | RESOLUTION RANGE HIGH | (ANGSTROMS) | : | 1.80 |
| REMARK | 3 | RESOLUTION RANGE LOW | (ANGSTROMS) | : | 81.65 |
| REMARK | 3 | DATA CUTOFF | (SIGMA (F)) | : | NONE |
| REMARK | 3 | COMPLETENESS FOR RANGE | (%) | : | 99.77 |
| REMARK | 3 | NUMBER OF REFLECTIONS | | : | 24820 |
| REMARK | 3 | | | | |

| | | |
|---|---|---|
| REMARK | 3 | FIT TO DATA USED IN REFINEMENT. |

| | | | | |
|---|---|---|---|---|
| REMARK | 3 | CROSS-VALIDATION METHOD | : | THROUGHOUT |
| REMARK | 3 | FREE R VALUE TEST SET SELECTION | : | RANDOM |

| | | | | | |
|---|---|---|---|---|---|
| REMARK | 3 | R VALUE | (WORKING + TEST SET) | : | 0.18829 |
| REMARK | 3 | R VALUE | (WORKING SET) | : | 0.18620 |

| REMARK | 3 | FREE R VALUE | | : | 0.22809 |
|---|---|---|---|---|---|
| REMARK | 3 | FREE R VALUE TEST SET SIZE | (%) | : | 5.1 |

| | | | | |
|---|---|---|---|---|
| REMARK | 3 | FREE R VALUE TEST SET COUNT | : | 1327 |
| REMARK | 3 | | | |

| | | |
|---|---|---|
| REMARK | 3 | FIT IN THE HIGHEST RESOLUTION BIN. |

| | | | | |
|---|---|---|---|---|
| REMARK | 3 | TOTAL NUMBER OF BINS USED | : | 20 |
| REMARK | 3 | BIN RESOLUTION RANGE HIGH | : | 1.800 |
| REMARK | 3 | BIN RESOLUTION RANGE LOW | : | 1.847 |

| | | | | | |
|---|---|---|---|---|---|
| REMARK | 3 | REFLECTION IN BIN | (WORKING SET) | : | 1749 |
| REMARK | 3 | BIN R VALUE | (WORKING SET) | : | 0.242 |

| | | | | |
|---|---|---|---|---|
| REMARK | 3 | BIN FREE R VALUE SET COUNT | : | 90 |
| REMARK | 3 | BIN FREE R VALUE | : | 0.288 |
| REMARK | 3 | | | |

| | | |
|---|---|---|
| REMARK | 3 | NUMBER OF NON-HYDROGEN ATOMS USED IN REFINEMENT. |

| | | | | |
|---|---|---|---|---|
| REMARK | 3 | ALL ATOMS | : | 2507 |

| | | |
|---|---|---|
| REMARK | 3 | |
| REMARK | 3 | B VALUES. |

| | | | | |
|---|---|---|---|---|
| REMARK | 3 | FROM WILSON PLOT | (A**2): | NULL |
| REMARK | 3 | MEAN B VALUE | (OVERALL, A**2): | 17.218 |

| | | |
|---|---|---|
| REMARK | 3 | OVERALL ANISOTROPIC B VALUE. |

| | | | |
|---|---|---|---|
| REMARK | 3 | B11 (A**2) : | −0.09 |
| REMARK | 3 | B22 (A**2) : | 0.14 |
| REMARK | 3 | B33 (A**2) : | −0.04 |
| REMARK | 3 | B12 (A**2) : | 0.00 |
| REMARK | 3 | B13 (A**2) : | −0.02 |
| REMARK | 3 | B23 (A**2) : | 0.00 |
| REMARK | 3 | | |

| | | |
|---|---|---|
| REMARK | 3 | ESTIMATED OVERALL COORDINATE ERROR. |

| | | | | |
|---|---|---|---|---|
| REMARK | 3 | ESU BASED ON R VALUE | (A): | 0.141 |
| REMARK | 3 | ESU BASED ON FREE R VALUE | (A): | 0.133 |
| REMARK | 3 | ESU BASED ON MAXIMUM LIKELIHOOD | (A): | 0.082 |

| | | | |
|---|---|---|---|
| REMARK | 3 | ESU FOR B VALUES BASED ON MAXIMUM LIKELIHOOD (A**2): | 2.620 |

| | |
|---|---|
| REMARK | 3 |

| | | | |
|---|---|---|---|
| REMARK | 3 | CORRELATION COEFFICIENTS. | |
| REMARK | 3 | CORRELATION COEFFICIENT FO-FC : | 0.948 |
| REMARK | 3 | CORRELATION COEFFICIENT FO-FC FREE : | 0.929 |

| REMARK | 3 | | | | |
|---|---|---|---|---|---|
| REMARK | 3 | RMS DEVIATIONS FROM IDEAL VALUES | COUNT | RMS | WEIGHT |

| | | | | | | |
|---|---|---|---|---|---|---|
| REMARK | 3 | BOND LENGTHS REFINED ATOMS | (A) : | 2310; | 0.010; | 0.022 |
| REMARK | 3 | BOND LENGTHS OTHERS | (A) : | 2097; | 0.002; | 0.020 |

| | | | | | | |
|---|---|---|---|---|---|---|
| REMARK | 3 | BOND ANGLES REFINED ATOMS | (DEGREES) : | 3134; | 1.372; | 1.981 |
| REMARK | 3 | BOND ANGLES OTHERS | (DEGREES) : | 4890; | 0.790; | 3.000 |
| REMARK | 3 | TORSION ANGLES, PERIOD 1 | (DEGREES) : | 272; | 5.281; | 5.000 |

| | | | | | | |
|---|---|---|---|---|---|---|
| REMARK | 3 | CHIRAL-CENTER RESTRAINTS | (A**3) : | 344; | 0.076; | 0.200 |
| REMARK | 3 | GENERAL PLANES REFINED ATOMS | (A) : | 2489; | 0.005; | 0.020 |
| REMARK | 3 | GENERAL PLANES OTHERS | (A) : | 465; | 0.002; | 0.020 |

| | | | | | | |
|---|---|---|---|---|---|---|
| REMARK | 3 | NON-BONDED CONTACTS REFINED | (A) : | 475; | 0.205; | 0.200 |
| | | ATOMS | | | | |
| REMARK | 3 | NON-BONDED CONTACTS OTHERS | (A) : | 2364; | 0.223; | 0.200 |
| REMARK | 3 | NON-BONDED TORSION OTHERS | (A) : | 1222; | 0.081; | 0.200 |
| REMARK | 3 | H-BOND (X...Y) REFINED ATOMS | (A) : | 147; | 0.162; | 0.200 |
| REMARK | 3 | SYMMETRY VDW REFINED ATOMS | (A) : | 21; | 0.168; | 0.200 |
| REMARK | 3 | SYMMETRY VDW OTHERS | (A) : | 86; | 0.250; | 0.200 |
| REMARK | 3 | SYMMETRY H-BOND REFINED ATOMS | (A) : | 14; | 0.111; | 0.200 |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |

| | | | | | | |
|---|---|---|---|---|---|---|
| REMARK | 3 | MAIN-CHAIN BOND REFINED | (A**2) : | 1365; | 0.818; | 1.500 |
| | | ATOMS | | | | |
| REMARK | 3 | MAIN-CHAIN ANGLE REFINED | (A**2) : | 2224; | 1.568; | 2.000 |
| | | ATOMS | | | | |
| REMARK | 3 | SIDE-CHAIN BOND REFINED | (A**2) : | 945; | 2.206; | 3.000 |
| | | ATOMS | | | | |
| REMARK | 3 | SIDE-CHAIN ANGLE REFINED | (A**2) : | 910; | 3.668; | 4.500 |
| | | ATOMS | | | | |
| REMARK | 3 | | | | | |

| | | |
|---|---|---|
| REMARK | 3 | NCS RESTRAINTS STATISTICS |

| | | |
|---|---|---|
| REMARK | 3 | NUMBER OF NCS GROUPS: NULL |
| REMARK | 3 | |
| REMARK | 3 | |

| | | |
|---|---|---|
| REMARK | 3 | TLS DETAILS |
| REMARK | 3 | NUMBER OF TLS GROUPS : 1 |
| REMARK | 3 | |
| REMARK | 3 | TLS GROUP : 1 |

| | | |
|---|---|---|
| REMARK | 3 | NUMBER OF COMPONENTS GROUP : 1 |
| REMARK | 3 | COMPONENTS C SSSEQI TO C SSSEQI |
| REMARK | 3 | RESIDUE RANGE : A 419 A 691 |
| REMARK | 3 | ORIGIN FOR THE GROUP (A): 6.9620 1.7680 19.1340 |
| REMARK | 3 | T TENSOR |

| | | | | | |
|---|---|---|---|---|---|
| REMARK | 3 | T11: | 0.0048 | T22: | 0.0352 |
| REMARK | 3 | T33: | 0.0580 | T12: | −0.0119 |
| REMARK | 3 | T13: | −0.0081 | T23: | 0.0084 |

| | | |
|---|---|---|
| REMARK | 3 | L TENSOR |

| | | | | | |
|---|---|---|---|---|---|
| REMARK | 3 | L11: | 0.3962 | L22: | 0.3784 |
| REMARK | 3 | L33: | 0.2902 | L12: | −0.1647 |
| REMARK | 3 | L13: | 0.0731 | L23: | 0.0592 |

| | | |
|---|---|---|
| REMARK | 3 | S TENSOR |

| | | | | | | | |
|---|---|---|---|---|---|---|---|
| REMARK | 3 | S11: | −0.0145 | S12: | 0.0246 | S13: | 0.0170 |
| REMARK | 3 | S21: | 0.0077 | S22: | 0.0410 | S23: | 0.0381 |
| REMARK | 3 | S31: | −0.0159 | S32: | 0.0355 | S33: | −0.0265 |
| REMARK | 3 | | | | | | |
| REMARK | 3 | | | | | | |

| | | |
|---|---|---|
| REMARK | 3 | BULK SOLVENT MODELLING. |

| | | |
|---|---|---|
| REMARK | 3 | METHOD USED : BABINET MODEL WITH MASK |
| REMARK | 3 | PARAMETERS FOR MASK CALCULATION |
| REMARK | 3 | VDW PROBE RADIUS : 1.40 |
| REMARK | 3 | ION PROBE RADIUS : 0.80 |
| REMARK | 3 | SHRINKAGE RADIUS : 0.80 |
| REMARK | 3 | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |

| | |
|---|---|
| CRYST1 | 37.316 46.978 81.109 90.00 92.83 90.00 P 1 21 1 |

| | | | | |
|---|---|---|---|---|
| SCALE1 | 0.026798 | 0.000000 | 0.001323 | 0.00000 |
| SCALE2 | 0.000000 | 0.021287 | 0.000000 | 0.00000 |
| SCALE3 | 0.000000 | 0.000000 | 0.012344 | 0.00000 |

| | | | | | | | | | | | | |
|---|---|---|---|---|---|---|---|---|---|---|---|---|
| ATOM | 1 | N | MET | A | 419 | −17.724 | 15.274 | 26.545 | 1.00 | 41.92 | A | N |
| ATOM | 3 | CA | MET | A | 419 | −16.798 | 15.014 | 25.404 | 1.00 | 41.87 | A | C |
| ATOM | 5 | CB | MET | A | 419 | −17.513 | 15.303 | 24.075 | 1.00 | 42.37 | A | C |
| ATOM | 8 | CG | MET | A | 419 | −18.905 | 14.692 | 23.955 | 1.00 | 44.21 | A | C |
| ATOM | 11 | SD | MET | A | 419 | −18.872 | 12.884 | 23.783 | 1.00 | 48.64 | A | S |
| ATOM | 12 | CE | MET | A | 419 | −19.036 | 12.354 | 25.521 | 1.00 | 47.82 | A | C |
| ATOM | 16 | C | MET | A | 419 | −15.527 | 15.875 | 25.505 | 1.00 | 40.85 | A | C |
| ATOM | 17 | O | MET | A | 419 | −14.857 | 16.115 | 24.495 | 1.00 | 41.39 | A | O |
| ATOM | 20 | N | ILE | A | 420 | −15.200 | 16.322 | 26.719 | 1.00 | 39.21 | A | N |
| ATOM | 22 | CA | ILE | A | 420 | −14.050 | 17.208 | 26.982 | 1.00 | 37.79 | A | C |
| ATOM | 24 | CB | ILE | A | 420 | −12.697 | 16.519 | 26.689 | 1.00 | 37.82 | A | C |
| ATOM | 26 | CG1 | ILE | A | 420 | −12.557 | 15.240 | 27.512 | 1.00 | 38.41 | A | C |
| ATOM | 29 | CD1 | ILE | A | 420 | −11.209 | 14.556 | 27.348 | 1.00 | 38.81 | A | C |
| ATOM | 33 | CG2 | ILE | A | 420 | −11.539 | 17.494 | 26.996 | 1.00 | 37.64 | A | C |
| ATOM | 37 | C | ILE | A | 420 | −14.081 | 18.526 | 26.218 | 1.00 | 36.12 | A | C |
| ATOM | 38 | O | ILE | A | 420 | −13.850 | 18.561 | 25.013 | 1.00 | 36.25 | A | O |
| ATOM | 39 | N | ALA | A | 421 | −14.316 | 19.613 | 26.935 | 1.00 | 34.13 | A | N |
| ATOM | 41 | CA | ALA | A | 421 | −14.207 | 20.936 | 26.356 | 1.00 | 32.57 | A | C |
| ATOM | 43 | CB | ALA | A | 421 | −15.126 | 21.909 | 27.076 | 1.00 | 32.58 | A | C |
| ATOM | 47 | C | ALA | A | 421 | −12.762 | 21.394 | 26.457 | 1.00 | 31.11 | A | C |
| ATOM | 48 | O | ALA | A | 421 | −12.009 | 20.935 | 27.315 | 1.00 | 30.48 | A | O |
| ATOM | 49 | N | ARG | A | 422 | −12.385 | 22.305 | 25.572 | 1.00 | 29.35 | A | N |
| ATOM | 51 | CA | ARG | A | 422 | −11.069 | 22.917 | 25.610 | 1.00 | 28.21 | A | C |
| ATOM | 53 | CB | ARG | A | 422 | −10.957 | 23.974 | 24.506 | 1.00 | 28.08 | A | C |
| ATOM | 56 | CG | ARG | A | 422 | −9.542 | 24.501 | 24.279 | 1.00 | 27.30 | A | C |
| ATOM | 59 | CD | ARG | A | 422 | −9.471 | 25.640 | 23.289 | 1.00 | 25.94 | A | C |
| ATOM | 62 | NE | ARG | A | 422 | −10.069 | 25.288 | 22.005 | 1.00 | 25.59 | A | N |
| ATOM | 64 | CZ | ARG | A | 422 | −9.474 | 24.572 | 21.057 | 1.00 | 24.52 | A | C |
| ATOM | 65 | NH1 | ARG | A | 422 | −8.241 | 24.095 | 21.225 | 1.00 | 22.64 | A | N |
| ATOM | 68 | NH2 | ARG | A | 422 | −10.124 | 24.320 | 19.932 | 1.00 | 24.29 | A | N |
| ATOM | 71 | C | ARG | A | 422 | −10.773 | 23.535 | 26.985 | 1.00 | 27.22 | A | C |
| ATOM | 72 | O | ARG | A | 422 | −9.632 | 23.519 | 27.435 | 1.00 | 26.74 | A | O |
| ATOM | 73 | N | GLU | A | 423 | −11.808 | 24.051 | 27.652 | 1.00 | 26.08 | A | N |
| ATOM | 75 | CA | GLU | A | 423 | −11.674 | 24.666 | 28.979 | 1.00 | 25.78 | A | C |
| ATOM | 77 | CB | GLU | A | 423 | −13.012 | 25.237 | 29.474 | 1.00 | 26.29 | A | C |
| ATOM | 80 | CG | GLU | A | 423 | −13.662 | 26.233 | 28.552 | 1.00 | 28.01 | A | C |
| ATOM | 83 | CD | GLU | A | 423 | −14.629 | 25.584 | 27.583 | 1.00 | 29.62 | A | C |
| ATOM | 84 | OE1 | GLU | A | 423 | −14.183 | 25.287 | 26.450 | 1.00 | 28.40 | A | O |
| ATOM | 85 | OE2 | GLU | A | 423 | −15.823 | 25.382 | 27.960 | 1.00 | 30.82 | A | O |
| ATOM | 86 | C | GLU | A | 423 | −11.224 | 23.675 | 30.040 | 1.00 | 24.55 | A | C |
| ATOM | 87 | O | GLU | A | 423 | −10.636 | 24.070 | 31.034 | 1.00 | 24.25 | A | O |
| ATOM | 88 | N | ASP | A | 424 | −11.550 | 22.401 | 29.843 | 1.00 | 23.65 | A | N |
| ATOM | 90 | CA | ASP | A | 424 | −11.151 | 21.351 | 30.778 | 1.00 | 23.18 | A | C |
| ATOM | 92 | CB | ASP | A | 424 | −11.925 | 20.056 | 30.503 | 1.00 | 23.13 | A | C |
| ATOM | 95 | CG | ASP | A | 424 | −13.436 | 20.219 | 30.670 | 1.00 | 25.17 | A | C |
| ATOM | 96 | OD1 | ASP | A | 424 | −13.848 | 21.127 | 31.427 | 1.00 | 26.20 | A | O |
| ATOM | 97 | OD2 | ASP | A | 424 | −14.276 | 19.481 | 30.095 | 1.00 | 26.16 | A | O |
| ATOM | 98 | C | ASP | A | 424 | −9.639 | 21.067 | 30.742 | 1.00 | 22.42 | A | C |
| ATOM | 99 | O | ASP | A | 424 | −9.148 | 20.360 | 31.606 | 1.00 | 21.99 | A | O |
| ATOM | 100 | N | VAL | A | 425 | −8.920 | 21.606 | 29.752 | 1.00 | 21.44 | A | N |
| ATOM | 102 | CA | VAL | A | 425 | −7.488 | 21.342 | 29.592 | 1.00 | 21.23 | A | C |
| ATOM | 104 | CB | VAL | A | 425 | −7.184 | 20.639 | 28.249 | 1.00 | 21.09 | A | C |
| ATOM | 106 | CG1 | VAL | A | 425 | −5.678 | 20.393 | 28.092 | 1.00 | 21.37 | A | C |
| ATOM | 110 | CG2 | VAL | A | 425 | −7.963 | 19.337 | 28.133 | 1.00 | 20.93 | A | C |
| ATOM | 114 | C | VAL | A | 425 | −6.715 | 22.641 | 29.649 | 1.00 | 21.24 | A | C |
| ATOM | 115 | O | VAL | A | 425 | −6.957 | 23.541 | 28.836 | 1.00 | 21.03 | A | O |
| ATOM | 116 | N | VAL | A | 426 | −5.824 | 22.742 | 30.631 | 1.00 | 20.75 | A | N |
| ATOM | 118 | CA | VAL | A | 426 | −4.965 | 23.894 | 30.830 | 1.00 | 21.32 | A | C |
| ATOM | 120 | CB | VAL | A | 426 | −4.992 | 24.367 | 32.300 | 1.00 | 21.30 | A | C |
| ATOM | 122 | CG1 | VAL | A | 426 | −4.044 | 25.545 | 32.514 | 1.00 | 22.52 | A | C |
| ATOM | 126 | CG2 | VAL | A | 426 | −6.415 | 24.743 | 32.718 | 1.00 | 21.78 | A | C |
| ATOM | 130 | C | VAL | A | 426 | −3.522 | 23.530 | 30.466 | 1.00 | 21.27 | A | C |
| ATOM | 131 | O | VAL | A | 426 | −2.931 | 22.621 | 31.046 | 1.00 | 20.65 | A | O |
| ATOM | 132 | N | LEU | A | 427 | −2.960 | 24.253 | 29.509 | 1.00 | 21.37 | A | N |
| ATOM | 134 | CA | LEU | A | 427 | −1.585 | 24.024 | 29.079 | 1.00 | 21.38 | A | C |
| ATOM | 136 | CB | LEU | A | 427 | −1.413 | 24.387 | 27.593 | 1.00 | 21.39 | A | C |
| ATOM | 139 | CG | LEU | A | 427 | −2.428 | 23.797 | 26.603 | 1.00 | 21.09 | A | C |
| ATOM | 141 | CD1 | LEU | A | 427 | −2.214 | 24.341 | 25.191 | 1.00 | 21.00 | A | C |
| ATOM | 145 | CD2 | LEU | A | 427 | −2.399 | 22.267 | 26.592 | 1.00 | 20.75 | A | C |
| ATOM | 149 | C | LEU | A | 427 | −0.626 | 24.841 | 29.931 | 1.00 | 22.15 | A | C |
| ATOM | 150 | O | LEU | A | 427 | −0.819 | 26.043 | 30.102 | 1.00 | 21.62 | A | O |
| ATOM | 151 | N | ASN | A | 428 | 0.413 | 24.189 | 30.448 | 1.00 | 22.43 | A | N |
| ATOM | 153 | CA | ASN | A | 428 | 1.416 | 24.834 | 31.311 | 1.00 | 23.45 | A | C |
| ATOM | 155 | CB | ASN | A | 428 | 1.710 | 23.923 | 32.508 | 1.00 | 23.76 | A | C |
| ATOM | 158 | CG | ASN | A | 428 | 0.458 | 23.579 | 33.293 | 1.00 | 26.13 | A | C |
| ATOM | 159 | OD1 | ASN | A | 428 | 0.301 | 22.454 | 33.774 | 1.00 | 30.61 | A | O |
| ATOM | 160 | ND2 | ASN | A | 428 | −0.455 | 24.536 | 33.400 | 1.00 | 28.03 | A | N |
| ATOM | 163 | C | ASN | A | 428 | 2.728 | 25.192 | 30.611 | 1.00 | 23.29 | A | C |
| ATOM | 164 | O | ASN | A | 428 | 3.316 | 26.231 | 30.907 | 1.00 | 23.11 | A | O |
| ATOM | 165 | N | ARG | A | 429 | 3.217 | 24.316 | 29.732 | 1.00 | 23.23 | A | N |
| ATOM | 167 | CA | ARG | A | 429 | 4.438 | 24.593 | 28.965 | 1.00 | 23.80 | A | C |
| ATOM | 169 | CB | ARG | A | 429 | 5.678 | 24.419 | 29.846 | 1.00 | 24.47 | A | C |
| ATOM | 172 | CG | ARG | A | 429 | 5.945 | 22.999 | 30.298 | 1.00 | 26.49 | A | C |
| ATOM | 175 | CD | ARG | A | 429 | 7.254 | 22.845 | 31.078 | 1.00 | 30.54 | A | C |
| ATOM | 178 | NE | ARG | A | 429 | 7.866 | 21.544 | 30.824 | 1.00 | 34.08 | A | N |
| ATOM | 180 | CZ | ARG | A | 429 | 8.873 | 21.315 | 29.980 | 1.00 | 35.96 | A | C |
| ATOM | 181 | NH1 | ARG | A | 429 | 9.438 | 22.308 | 29.290 | 1.00 | 36.94 | A | N |
| ATOM | 184 | NH2 | ARG | A | 429 | 9.330 | 20.077 | 29.838 | 1.00 | 36.82 | A | N |
| ATOM | 187 | C | ARG | A | 429 | 4.596 | 23.731 | 27.715 | 1.00 | 23.63 | A | C |
| ATOM | 188 | O | ARG | A | 429 | 3.847 | 22.785 | 27.506 | 1.00 | 22.57 | A | O |
| ATOM | 189 | N | ILE | A | 430 | 5.592 | 24.053 | 26.895 | 1.00 | 23.59 | A | N |
| ATOM | 191 | CA | ILE | A | 430 | 5.901 | 23.258 | 25.711 | 1.00 | 24.18 | A | C |
| ATOM | 193 | CB | ILE | A | 430 | 6.382 | 24.161 | 24.535 | 1.00 | 24.28 | A | C |
| ATOM | 195 | CG1 | ILE | A | 430 | 5.211 | 25.021 | 24.046 | 1.00 | 24.59 | A | C |
| ATOM | 198 | CD1 | ILE | A | 430 | 5.513 | 25.900 | 22.844 | 1.00 | 23.92 | A | C |
| ATOM | 202 | CG2 | ILE | A | 430 | 6.967 | 23.305 | 23.398 | 1.00 | 24.51 | A | C |
| ATOM | 206 | C | ILE | A | 430 | 6.914 | 22.169 | 26.036 | 1.00 | 25.15 | A | C |
| ATOM | 207 | O | ILE | A | 430 | 7.978 | 22.435 | 26.594 | 1.00 | 25.52 | A | O |
| ATOM | 208 | N | LEU | A | 431 | 6.555 | 20.940 | 25.683 | 1.00 | 26.06 | A | N |
| ATOM | 210 | CA | LEU | A | 431 | 7.351 | 19.745 | 25.935 | 1.00 | 27.29 | A | C |
| ATOM | 212 | CB | LEU | A | 431 | 6.411 | 18.533 | 25.929 | 1.00 | 27.83 | A | C |
| ATOM | 215 | CG | LEU | A | 431 | 6.627 | 17.314 | 26.814 | 1.00 | 29.05 | A | C |
| ATOM | 217 | CD1 | LEU | A | 431 | 7.002 | 17.675 | 28.242 | 1.00 | 29.90 | A | C |
| ATOM | 221 | CD2 | LEU | A | 431 | 5.346 | 16.505 | 26.789 | 1.00 | 29.38 | A | C |
| ATOM | 225 | C | LEU | A | 431 | 8.423 | 19.572 | 24.863 | 1.00 | 28.18 | A | C |
| ATOM | 226 | O | LEU | A | 431 | 9.582 | 19.243 | 25.149 | 1.00 | 28.38 | A | O |
| ATOM | 227 | N | GLY | A | 432 | 8.024 | 19.793 | 23.620 | 1.00 | 28.92 | A | N |
| ATOM | 229 | CA | GLY | A | 432 | 8.932 | 19.691 | 22.500 | 1.00 | 29.66 | A | C |
| ATOM | 232 | C | GLY | A | 432 | 8.267 | 19.963 | 21.166 | 1.00 | 30.32 | A | C |
| ATOM | 233 | O | GLY | A | 432 | 7.040 | 20.023 | 21.065 | 1.00 | 29.86 | A | O |
| ATOM | 234 | N | GLU | A | 433 | 9.096 | 20.144 | 20.144 | 1.00 | 31.32 | A | N |
| ATOM | 236 | CA | GLU | A | 433 | 8.636 | 20.260 | 18.767 | 1.00 | 32.42 | A | C |
| ATOM | 238 | CB | GUU | A | 433 | 9.600 | 21.120 | 17.946 | 1.00 | 33.03 | A | C |
| ATOM | 241 | CG | GLU | A | 433 | 9.426 | 21.056 | 16.433 | 1.00 | 35.97 | A | C |
| ATOM | 244 | CD | GLU | A | 433 | 8.136 | 21.696 | 15.966 | 1.00 | 39.52 | A | C |
| ATOM | 245 | OE1 | GLU | A | 433 | 7.078 | 21.062 | 16.142 | 1.00 | 41.45 | A | O |
| ATOM | 246 | OE2 | GUU | A | 433 | 8.178 | 22.828 | 15.417 | 1.00 | 43.45 | A | O |
| ATOM | 247 | C | GLU | A | 433 | 8.559 | 18.853 | 18.200 | 1.00 | 32.62 | A | C |
| ATOM | 248 | O | GLU | A | 433 | 9.584 | 18.218 | 17.959 | 1.00 | 33.07 | A | O |
| ATOM | 249 | N | GLY | A | 434 | 7.341 | 18.364 | 18.027 | 1.00 | 32.46 | A | N |
| ATOM | 251 | CA | GLY | A | 434 | 7.110 | 17.080 | 17.411 | 1.00 | 32.53 | A | C |
| ATOM | 254 | C | GLY | A | 434 | 6.926 | 17.141 | 15.904 | 1.00 | 32.66 | A | C |
| ATOM | 255 | O | GLY | A | 434 | 7.033 | 18.193 | 15.266 | 1.00 | 32.19 | A | O |
| ATOM | 256 | N | PHE | A | 435 | 6.619 | 15.974 | 15.353 | 1.00 | 32.83 | A | N |
| ATOM | 258 | CA | PHE | A | 435 | 6.413 | 15.765 | 13.926 | 1.00 | 32.68 | A | C |
| ATOM | 260 | CB | PHE | A | 435 | 6.032 | 14.294 | 13.703 | 1.00 | 33.30 | A | C |
| ATOM | 263 | CG | PHE | A | 435 | 6.016 | 13.890 | 12.267 | 1.00 | 35.29 | A | C |
| ATOM | 264 | CD1 | PHE | A | 435 | 7.202 | 13.628 | 11.601 | 1.00 | 37.64 | A | C |
| ATOM | 266 | CE1 | PHE | A | 435 | 7.197 | 13.260 | 10.263 | 1.00 | 38.62 | A | C |
| ATOM | 268 | CZ | PHE | A | 435 | 6.000 | 13.162 | 9.582 | 1.00 | 38.93 | A | C |
| ATOM | 270 | CE2 | PHE | A | 435 | 4.807 | 13.434 | 10.236 | 1.00 | 38.98 | A | C |
| ATOM | 272 | CD2 | PHE | A | 435 | 4.821 | 13.794 | 11.575 | 1.00 | 36.99 | A | C |
| ATOM | 274 | C | PHE | A | 435 | 5.330 | 16.662 | 13.319 | 1.00 | 31.77 | A | C |
| ATOM | 275 | O | PHE | A | 435 | 5.533 | 17.269 | 12.262 | 1.00 | 31.44 | A | O |
| ATOM | 276 | N | PHE | A | 436 | 4.178 | 16.734 | 13.982 | 1.00 | 30.64 | A | N |
| ATOM | 278 | CA | PHE | A | 436 | 3.030 | 17.476 | 13.465 | 1.00 | 29.83 | A | C |
| ATOM | 280 | CB | PHE | A | 436 | 1.716 | 16.792 | 13.880 | 1.00 | 30.65 | A | C |
| ATOM | 283 | CG | PHE | A | 436 | 1.425 | 15.511 | 13.114 | 1.00 | 33.59 | A | C |
| ATOM | 284 | CD1 | PHE | A | 436 | 0.993 | 15.554 | 11.791 | 1.00 | 36.89 | A | C |
| ATOM | 286 | CE1 | PHE | A | 436 | 0.731 | 14.368 | 11.076 | 1.00 | 38.35 | A | C |
| ATOM | 288 | CZ | PHE | A | 436 | 0.917 | 13.136 | 11.692 | 1.00 | 38.06 | A | C |
| ATOM | 290 | CE2 | PHE | A | 436 | 1.354 | 13.086 | 13.006 | 1.00 | 37.74 | A | C |
| ATOM | 292 | CD2 | PHE | A | 436 | 1.608 | 14.268 | 13.709 | 1.00 | 36.81 | A | C |
| ATOM | 294 | C | PHE | A | 436 | 3.036 | 18.949 | 13.904 | 1.00 | 27.92 | A | C |
| ATOM | 295 | O | PHE | A | 436 | 2.435 | 19.783 | 13.242 | 1.00 | 26.90 | A | O |
| ATOM | 296 | N | GLY | A | 437 | 3.713 | 19.242 | 15.021 | 1.00 | 25.85 | A | N |
| ATOM | 298 | CA | GLY | A | 437 | 3.773 | 20.582 | 15.598 | 1.00 | 24.35 | A | C |
| ATOM | 301 | C | GLY | A | 437 | 4.152 | 20.555 | 17.076 | 1.00 | 23.03 | A | C |
| ATOM | 302 | O | GLY | A | 437 | 4.635 | 19.549 | 17.596 | 1.00 | 22.52 | A | O |
| ATOM | 303 | N | GLU | A | 438 | 3.912 | 21.660 | 17.774 | 1.00 | 21.56 | A | N |
| ATOM | 305 | CA | GLU | A | 438 | 4.295 | 21.761 | 19.176 | 1.00 | 20.35 | A | C |
| ATOM | 307 | CB | GLU | A | 438 | 4.114 | 23.191 | 19.694 | 1.00 | 20.82 | A | C |
| ATOM | 310 | CG | GLU | A | 438 | 4.921 | 24.235 | 18.922 | 1.00 | 23.31 | A | C |
| ATOM | 313 | CD | GLU | A | 438 | 6.325 | 24.443 | 19.459 | 1.00 | 26.75 | A | C |
| ATOM | 314 | OE1 | GLU | A | 438 | 6.960 | 23.454 | 19.896 | 1.00 | 24.49 | A | O |
| ATOM | 315 | OE2 | GLU | A | 438 | 6.792 | 25.611 | 19.446 | 1.00 | 29.56 | A | O |
| ATOM | 316 | C | GLU | A | 438 | 3.473 | 20.781 | 20.016 | 1.00 | 18.49 | A | C |
| ATOM | 317 | O | GLU | A | 438 | 2.306 | 20.552 | 19.744 | 1.00 | 17.45 | A | O |
| ATOM | 318 | N | VAL | A | 439 | 4.124 | 20.199 | 21.017 | 1.00 | 17.29 | A | N |
| ATOM | 320 | CA | VAL | A | 439 | 3.498 | 19.315 | 21.986 | 1.00 | 16.28 | A | C |
| ATOM | 322 | CB | VAL | A | 439 | 4.183 | 17.933 | 22.024 | 1.00 | 16.57 | A | C |
| ATOM | 324 | CG1 | VAL | A | 439 | 3.516 | 17.038 | 23.065 | 1.00 | 17.25 | A | C |
| ATOM | 328 | CG2 | VAL | A | 439 | 4.141 | 17.301 | 20.643 | 1.00 | 16.55 | A | C |
| ATOM | 332 | C | VAL | A | 439 | 3.623 | 19.974 | 23.350 | 1.00 | 15.68 | A | C |
| ATOM | 333 | O | VAL | A | 439 | 4.705 | 20.380 | 23.732 | 1.00 | 14.39 | A | O |
| ATOM | 334 | N | TYR | A | 440 | 2.508 | 20.084 | 24.068 | 1.00 | 14.79 | A | N |
| ATOM | 336 | CA | TYR | A | 440 | 2.447 | 20.789 | 25.345 | 1.00 | 14.71 | A | C |
| ATOM | 338 | CB | TYR | A | 440 | 1.269 | 21.749 | 25.334 | 1.00 | 14.65 | A | C |
| ATOM | 341 | CG | TYR | A | 440 | 1.348 | 22.842 | 24.301 | 1.00 | 15.25 | A | C |
| ATOM | 342 | CD1 | TYR | A | 440 | 1.809 | 24.110 | 24.639 | 1.00 | 16.36 | A | C |
| ATOM | 344 | CE1 | TYR | A | 440 | 1.860 | 25.135 | 23.691 | 1.00 | 16.17 | A | C |
| ATOM | 346 | CZ | TYR | A | 440 | 1.436 | 24.895 | 22.395 | 1.00 | 18.28 | A | C |
| ATOM | 347 | OH | TYR | A | 440 | 1.490 | 25.905 | 21.453 | 1.00 | 20.25 | A | O |
| ATOM | 349 | CE2 | TYR | A | 440 | 0.967 | 23.644 | 22.038 | 1.00 | 16.17 | A | C |
| ATOM | 351 | CD2 | TYR | A | 440 | 0.916 | 22.630 | 22.994 | 1.00 | 14.61 | A | C |
| ATOM | 353 | C | TYR | A | 440 | 2.228 | 19.817 | 26.479 | 1.00 | 15.34 | A | C |
| ATOM | 354 | O | TYR | A | 440 | 1.608 | 18.765 | 26.270 | 1.00 | 15.00 | A | O |
| ATOM | 355 | N | GLU | A | 441 | 2.723 | 20.168 | 27.669 | 1.00 | 15.04 | A | N |
| ATOM | 357 | CA | GLU | A | 441 | 2.340 | 19.514 | 28.921 | 1.00 | 16.43 | A | C |
| ATOM | 359 | CB | GLU | A | 441 | 3.513 | 19.470 | 29.916 | 1.00 | 17.43 | A | C |
| ATOM | 362 | CG | GLU | A | 441 | 3.244 | 18.575 | 31.117 | 1.00 | 21.96 | A | C |
| ATOM | 365 | CD | GLU | A | 441 | 4.331 | 18.631 | 32.173 | 1.00 | 28.06 | A | C |
| ATOM | 366 | OE1 | GLU | A | 441 | 5.533 | 18.478 | 31.828 | 1.00 | 31.85 | A | O |
| ATOM | 367 | OE2 | GLU | A | 441 | 3.975 | 18.829 | 33.361 | 1.00 | 32.28 | A | O |
| ATOM | 368 | C | GLU | A | 441 | 1.196 | 20.317 | 29.514 | 1.00 | 15.91 | A | C |
| ATOM | 369 | O | GLU | A | 441 | 1.205 | 21.549 | 29.457 | 1.00 | 15.73 | A | O |
| ATOM | 370 | N | GLY | A | 442 | 0.214 | 19.627 | 30.074 | 1.00 | 15.38 | A | N |
| ATOM | 372 | CA | GLY | A | 442 | −0.938 | 20.279 | 30.679 | 1.00 | 15.56 | A | C |
| ATOM | 375 | C | GLY | A | 442 | −1.686 | 19.408 | 31.671 | 1.00 | 15.46 | A | C |
| ATOM | 376 | O | GLY | A | 442 | −1.266 | 18.295 | 31.998 | 1.00 | 14.91 | A | O |
| ATOM | 377 | N | VAL | A | 443 | −2.799 | 19.937 | 32.159 | 1.00 | 15.90 | A | N |
| ATOM | 379 | CA | VAL | A | 443 | −3.655 | 19.250 | 33.120 | 1.00 | 15.95 | A | C |
| ATOM | 381 | CB | VAL | A | 443 | −3.585 | 19.926 | 34.515 | 1.00 | 15.99 | A | C |
| ATOM | 383 | CG1 | VAL | A | 443 | −4.535 | 19.256 | 35.486 | 1.00 | 16.92 | A | C |
| ATOM | 387 | CG2 | VAL | A | 443 | −2.159 | 19.901 | 35.054 | 1.00 | 16.66 | A | C |
| ATOM | 391 | C | VAL | A | 443 | −5.088 | 19.235 | 32.606 | 1.00 | 16.11 | A | C |
| ATOM | 392 | O | VAL | A | 443 | −5.677 | 20.280 | 32.328 | 1.00 | 16.39 | A | O |
| ATOM | 393 | N | TYR | A | 444 | −5.645 | 18.039 | 32.474 | 1.00 | 16.07 | A | N |
| ATOM | 395 | CA | TYR | A | 444 | −7.053 | 17.833 | 32.189 | 1.00 | 16.30 | A | C |
| ATOM | 397 | CB | TYR | A | 444 | −7.210 | 16.696 | 31.179 | 1.00 | 16.55 | A | C |
| ATOM | 400 | CG | TYR | A | 444 | −8.608 | 16.106 | 31.085 | 1.00 | 16.49 | A | C |
| ATOM | 401 | CD1 | TYR | A | 444 | −9.735 | 16.925 | 30.992 | 1.00 | 17.40 | A | C |
| ATOM | 403 | CE1 | TYR | A | 444 | −11.002 | 16.376 | 30.907 | 1.00 | 18.98 | A | C |
| ATOM | 405 | CZ | TYR | A | 444 | −11.159 | 15.013 | 30.900 | 1.00 | 19.67 | A | C |
| ATOM | 406 | OH | TYR | A | 444 | −12.417 | 14.454 | 30.818 | 1.00 | 22.12 | A | O |
| ATOM | 408 | CE2 | TYR | A | 444 | −10.066 | 14.189 | 30.992 | 1.00 | 18.71 | A | C |
| ATOM | 410 | CD2 | TYR | A | 444 | −8.801 | 14.737 | 31.070 | 1.00 | 18.30 | A | C |
| ATOM | 412 | C | TYR | A | 444 | −7.828 | 17.530 | 33.495 | 1.00 | 16.91 | A | C |
| ATOM | 413 | O | TYR | A | 444 | −7.500 | 16.593 | 34.223 | 1.00 | 16.06 | A | O |
| ATOM | 414 | N | THR | A | 445 | −8.845 | 18.338 | 33.779 | 1.00 | 17.53 | A | N |
| ATOM | 416 | CA | THR | A | 445 | −9.703 | 18.146 | 34.944 | 1.00 | 18.31 | A | C |
| ATOM | 418 | CB | THR | A | 445 | −9.911 | 19.475 | 35.684 | 1.00 | 18.17 | A | C |
| ATOM | 420 | OG1 | THR | A | 445 | −8.651 | 20.045 | 36.030 | 1.00 | 19.23 | A | O |
| ATOM | 422 | CG2 | THR | A | 445 | −10.578 | 19.262 | 37.044 | 1.00 | 18.29 | A | C |
| ATOM | 426 | C | THR | A | 445 | −11.032 | 17.632 | 34.430 | 1.00 | 19.18 | A | C |
| ATOM | 427 | O | THR | A | 445 | −11.702 | 18.345 | 33.694 | 1.00 | 19.17 | A | O |
| ATOM | 428 | N | ASN | A | 466 | −11.402 | 16.403 | 34.791 | 1.00 | 19.93 | A | N |
| ATOM | 430 | CA | ASN | A | 466 | −12.665 | 15.825 | 34.351 | 1.00 | 21.09 | A | C |
| ATOM | 432 | CB | ASN | A | 466 | −12.617 | 14.266 | 34.318 | 1.00 | 21.05 | A | C |
| ATOM | 435 | CG | ASN | A | 466 | −12.502 | 13.602 | 35.704 | 1.00 | 21.28 | A | C |
| ATOM | 436 | OD1 | ASN | A | 466 | −12.158 | 12.407 | 35.796 | 1.00 | 22.39 | A | O |
| ATOM | 437 | ND2 | ASN | A | 466 | −12.778 | 14.345 | 36.766 | 1.00 | 18.07 | A | N |
| ATOM | 440 | C | ASN | A | 466 | −13.831 | 16.414 | 35.163 | 1.00 | 22.07 | A | C |
| ATOM | 441 | O | ASN | A | 466 | −13.646 | 17.396 | 35.907 | 1.00 | 22.06 | A | O |
| ATOM | 442 | N | HIS | A | 447 | −15.025 | 15.861 | 34.999 | 1.00 | 23.22 | A | N |
| ATOM | 444 | CA | HIS | A | 447 | −16.215 | 16.466 | 35.621 | 1.00 | 24.56 | A | C |
| ATOM | 446 | CB | HIS | A | 447 | −17.479 | 16.009 | 34.878 | 1.00 | 25.52 | A | C |
| ATOM | 449 | CG | HIS | A | 447 | −17.560 | 16.535 | 33.474 | 1.00 | 28.90 | A | C |
| ATOM | 450 | ND1 | HIS | A | 447 | −17.485 | 17.879 | 33.177 | 1.00 | 32.01 | A | N |
| ATOM | 452 | CE1 | HIS | A | 447 | −17.581 | 18.047 | 31.869 | 1.00 | 32.90 | A | C |
| ATOM | 454 | NE2 | HIS | A | 447 | −17.712 | 16.859 | 31.306 | 1.00 | 33.63 | A | N |
| ATOM | 456 | CD2 | HIS | A | 447 | −17.703 | 15.896 | 32.289 | 1.00 | 32.36 | A | C |
| ATOM | 458 | C | HIS | A | 447 | −16.323 | 16.121 | 37.113 | 1.00 | 24.17 | A | C |
| ATOM | 459 | O | HIS | A | 447 | −17.141 | 16.715 | 37.819 | 1.00 | 24.60 | A | O |
| ATOM | 460 | N | LYS | A | 488 | −15.513 | 15.168 | 37.575 | 1.00 | 23.56 | A | N |
| ATOM | 462 | CA | LYS | A | 488 | −15.440 | 14.802 | 38.994 | 1.00 | 23.13 | A | C |
| ATOM | 464 | CB | LYS | A | 488 | −15.188 | 13.302 | 39.137 | 1.00 | 23.02 | A | C |
| ATOM | 467 | CG | LYS | A | 488 | −16.354 | 12.461 | 38.679 | 1.00 | 23.56 | A | C |
| ATOM | 470 | CD | LYS | A | 488 | −15.916 | 11.043 | 38.364 | 1.00 | 24.68 | A | C |
| ATOM | 473 | CE | LYS | A | 488 | −17.090 | 10.180 | 37.907 | 1.00 | 25.53 | A | C |
| ATOM | 476 | NZ | LYS | A | 488 | −16.689 | 8.742 | 37.931 | 1.00 | 23.92 | A | N |
| ATOM | 480 | C | LYS | A | 488 | −14.353 | 15.561 | 39.758 | 1.00 | 22.72 | A | C |
| ATOM | 481 | O | LYS | A | 488 | −14.197 | 15.369 | 40.974 | 1.00 | 23.18 | A | O |
| ATOM | 482 | N | GLY | A | 449 | −13.606 | 16.401 | 39.048 | 1.00 | 21.47 | A | N |
| ATOM | 484 | CA | GLY | A | 449 | −12.580 | 17.234 | 39.648 | 1.00 | 21.53 | A | C |
| ATOM | 487 | C | GLY | A | 449 | −11.237 | 16.542 | 39.729 | 1.00 | 21.19 | A | C |
| ATOM | 488 | O | GLY | A | 449 | −10.319 | 17.047 | 40.366 | 1.00 | 20.74 | A | O |
| ATOM | 489 | N | GLU | A | 450 | −11.127 | 15.386 | 39.080 | 1.00 | 20.98 | A | N |
| ATOM | 491 | CA | GLU | A | 450 | −9.879 | 14.630 | 39.054 | 1.00 | 21.35 | A | C |
| ATOM | 493 | CB | GLU | A | 450 | −10.150 | 13.172 | 38.691 | 1.00 | 21.63 | A | C |
| ATOM | 496 | CG | GLU | A | 450 | −11.186 | 12.486 | 39.565 | 1.00 | 23.33 | A | C |
| ATOM | 499 | CD | GLU | A | 450 | −11.347 | 11.024 | 39.214 | 1.00 | 26.08 | A | C |
| ATOM | 500 | OE1 | GLU | A | 450 | −10.821 | 10.178 | 39.963 | 1.00 | 26.94 | A | O |
| ATOM | 501 | OE2 | GLU | A | 450 | −12.024 | 10.726 | 38.201 | 1.00 | 28.51 | A | O |
| ATOM | 502 | C | GLU | A | 450 | −8.955 | 15.232 | 38.019 | 1.00 | 21.24 | A | C |
| ATOM | 503 | O | GLU | A | 450 | −9.376 | 15.478 | 36.902 | 1.00 | 20.28 | A | O |
| ATOM | 504 | N | LYS | A | 451 | −7.691 | 15.436 | 38.388 | 1.00 | 21.76 | A | N |
| ATOM | 506 | CA | LYS | A | 451 | −6.694 | 16.059 | 37.507 | 1.00 | 21.92 | A | C |
| ATOM | 508 | CB | LYS | A | 451 | −5.963 | 17.166 | 38.254 | 1.00 | 22.51 | A | C |
| ATOM | 511 | CG | LYS | A | 451 | −6.919 | 18.230 | 38.784 | 1.00 | 23.99 | A | C |
| ATOM | 514 | CD | LYS | A | 451 | −6.239 | 19.546 | 39.068 | 1.00 | 26.30 | A | C |
| ATOM | 517 | CE | LYS | A | 451 | −7.250 | 20.582 | 39.558 | 1.00 | 27.02 | A | C |
| ATOM | 520 | NZ | LYS | A | 451 | −7.947 | 21.286 | 38.459 | 1.00 | 26.84 | A | N |
| ATOM | 524 | C | LYS | A | 451 | −5.707 | 15.026 | 36.956 | 1.00 | 21.70 | A | C |
| ATOM | 525 | O | LYS | A | 451 | −5.111 | 14.249 | 37.707 | 1.00 | 22.12 | A | O |
| ATOM | 526 | N | ILE | A | 452 | −5.586 | 15.005 | 35.633 | 1.00 | 20.88 | A | N |
| ATOM | 528 | CA | ILE | A | 452 | −4.729 | 14.074 | 34.923 | 1.00 | 20.89 | A | C |
| ATOM | 530 | CB | ILE | A | 452 | −5.587 | 13.104 | 34.051 | 1.00 | 21.84 | A | C |
| ATOM | 532 | CG1 | ILE | A | 452 | −4.773 | 11.937 | 33.512 | 1.00 | 24.27 | A | C |
| ATOM | 535 | CD1 | ILE | A | 452 | −4.557 | 10.839 | 34.539 | 1.00 | 27.03 | A | C |
| ATOM | 539 | CG2 | ILE | A | 452 | −6.222 | 13.783 | 32.894 | 1.00 | 23.92 | A | C |
| ATOM | 543 | C | ILE | A | 452 | −3.743 | 14.883 | 34.086 | 1.00 | 19.23 | A | C |
| ATOM | 544 | O | ILE | A | 452 | −4.127 | 15.801 | 33.366 | 1.00 | 17.19 | A | O |
| ATOM | 545 | N | ASN | A | 453 | −2.468 | 14.561 | 34.227 | 1.00 | 18.03 | A | N |
| ATOM | 547 | CA | ASN | A | 453 | −1.427 | 15.173 | 33.421 | 1.00 | 16.96 | A | C |
| ATOM | 549 | CB | ASN | A | 453 | −0.039 | 14.893 | 34.019 | 1.00 | 17.63 | A | C |
| ATOM | 552 | CG | ASN | A | 453 | 0.144 | 15.546 | 35.375 | 1.00 | 19.82 | A | C |
| ATOM | 553 | OD1 | ASN | A | 453 | −0.283 | 16.675 | 35.581 | 1.00 | 20.59 | A | O |
| ATOM | 554 | ND2 | ASN | A | 453 | 0.782 | 14.835 | 36.307 | 1.00 | 22.47 | A | N |
| ATOM | 557 | C | ASN | A | 453 | −1.537 | 14.640 | 32.002 | 1.00 | 15.39 | A | C |
| ATOM | 558 | O | ASN | A | 453 | −1.741 | 13.432 | 31.792 | 1.00 | 13.98 | A | O |
| ATOM | 559 | N | VAL | A | 454 | −1.442 | 15.553 | 31.040 | 1.00 | 13.24 | A | N |
| ATOM | 561 | CA | VAL | A | 454 | −1.609 | 15.230 | 29.630 | 1.00 | 12.28 | A | C |
| ATOM | 563 | CB | VAL | A | 454 | −3.001 | 15.671 | 29.107 | 1.00 | 11.15 | A | C |
| ATOM | 565 | CG1 | VAL | A | 454 | −4.107 | 14.882 | 29.798 | 1.00 | 11.44 | A | C |
| ATOM | 569 | CG2 | VAL | A | 454 | −3.210 | 17.193 | 29.273 | 1.00 | 10.74 | A | C |
| ATOM | 573 | C | VAL | A | 454 | −0.515 | 15.843 | 28.752 | 1.00 | 12.12 | A | C |
| ATOM | 574 | O | VAL | A | 454 | 0.128 | 16.843 | 29.123 | 1.00 | 11.68 | A | O |
| ATOM | 575 | N | ALA | A | 455 | −0.276 | 15.196 | 27.615 | 1.00 | 11.65 | A | N |
| ATOM | 577 | CA | ALA | A | 455 | 0.496 | 15.770 | 26.521 | 1.00 | 11.60 | A | C |
| ATOM | 579 | CB | ALA | A | 455 | 1.493 | 14.785 | 25.979 | 1.00 | 12.08 | A | C |
| ATOM | 583 | C | ALA | A | 455 | −0.511 | 16.142 | 25.435 | 1.00 | 12.46 | A | C |
| ATOM | 584 | O | ALA | A | 455 | −1.331 | 15.327 | 25.055 | 1.00 | 12.88 | A | O |
| ATOM | 585 | N | VAL | A | 456 | −0.459 | 17.384 | 24.968 | 1.00 | 11.82 | A | N |
| ATOM | 587 | CA | VAL | A | 456 | −1.368 | 17.858 | 23.960 | 1.00 | 12.46 | A | C |
| ATOM | 589 | CB | VAL | A | 456 | −2.073 | 19.125 | 24.456 | 1.00 | 12.62 | A | C |
| ATOM | 591 | CG1 | VAL | A | 456 | −2.956 | 19.704 | 23.364 | 1.00 | 13.07 | A | C |
| ATOM | 595 | CG2 | VAL | A | 456 | −2.883 | 18.786 | 25.679 | 1.00 | 12.74 | A | C |
| ATOM | 599 | C | VAL | A | 456 | −0.622 | 18.148 | 22.668 | 1.00 | 13.13 | A | C |
| ATOM | 600 | O | VAL | A | 456 | 0.252 | 19.006 | 22.631 | 1.00 | 12.69 | A | O |
| ATOM | 601 | N | LYS | A | 457 | −0.958 | 17.415 | 21.616 | 1.00 | 13.43 | A | N |
| ATOM | 603 | CA | LYS | A | 457 | −0.324 | 17.577 | 20.322 | 1.00 | 14.66 | A | C |
| ATOM | 605 | CB | LYS | A | 457 | −0.214 | 16.220 | 19.615 | 1.00 | 15.55 | A | C |
| ATOM | 608 | CG | LYS | A | 457 | 0.694 | 15.217 | 20.350 | 1.00 | 18.42 | A | C |
| ATOM | 611 | CD | LYS | A | 457 | 0.840 | 13.874 | 19.626 | 1.00 | 22.96 | A | C |
| ATOM | 614 | CE | LYS | A | 457 | 1.221 | 14.008 | 18.151 | 1.00 | 26.16 | A | C |
| ATOM | 617 | NZ | LYS | A | 457 | 1.738 | 12.702 | 17.608 | 1.00 | 29.52 | A | N |
| ATOM | 621 | C | LYS | A | 457 | −1.123 | 18.569 | 19.480 | 1.00 | 14.79 | A | C |
| ATOM | 622 | O | LYS | A | 457 | −2.350 | 18.553 | 19.483 | 1.00 | 14.94 | A | O |
| ATOM | 623 | N | THR | A | 458 | −0.421 | 19.452 | 18.787 | 1.00 | 16.01 | A | N |
| ATOM | 625 | CA | THR | A | 458 | −1.056 | 20.426 | 17.902 | 1.00 | 17.14 | A | C |
| ATOM | 627 | CB | THR | A | 458 | −0.974 | 21.856 | 18.470 | 1.00 | 17.23 | A | C |
| ATOM | 629 | OG1 | THR | A | 458 | 0.390 | 22.295 | 18.492 | 1.00 | 16.75 | A | O |
| ATOM | 631 | CG2 | THR | A | 458 | −1.437 | 21.907 | 19.927 | 1.00 | 18.01 | A | C |
| ATOM | 635 | C | THR | A | 458 | −0.380 | 20.398 | 16.541 | 1.00 | 18.40 | A | C |
| ATOM | 636 | O | THR | A | 458 | 0.705 | 19.846 | 16.397 | 1.00 | 18.01 | A | O |
| ATOM | 637 | N | CYS | A | 459 | −1.032 | 21.017 | 15.561 | 1.00 | 20.44 | A | N |
| ATOM | 639 | CA | CYS | A | 459 | −0.512 | 21.123 | 14.195 | 1.00 | 22.30 | A | C |
| ATOM | 641 | CB | CYS | A | 459 | −1.643 | 20.944 | 13.189 | 1.00 | 22.75 | A | C |
| ATOM | 644 | SG | CYS | A | 459 | −2.097 | 19.219 | 12.971 | 1.00 | 27.79 | A | S |
| ATOM | 645 | C | CYS | A | 459 | 0.174 | 22.470 | 13.959 | 1.00 | 22.93 | A | C |
| ATOM | 646 | O | CYS | A | 459 | −0.344 | 23.509 | 14.347 | 1.00 | 22.16 | A | O |
| ATOM | 647 | N | LYS | A | 460 | 1.347 | 22.426 | 13.337 | 1.00 | 24.00 | A | N |
| ATOM | 649 | CA | LYS | A | 460 | 2.082 | 23.624 | 12.970 | 1.00 | 25.15 | A | C |
| ATOM | 651 | CB | LYS | A | 460 | 3.487 | 23.276 | 12.461 | 1.00 | 25.42 | A | C |
| ATOM | 654 | CG | LYS | A | 460 | 3.540 | 22.407 | 11.195 | 1.00 | 27.60 | A | C |
| ATOM | 657 | CD | LYS | A | 460 | 4.982 | 22.089 | 10.797 | 1.00 | 31.10 | A | C |
| ATOM | 660 | CE | LYS | A | 460 | 5.641 | 21.091 | 11.756 | 1.00 | 33.59 | A | C |
| ATOM | 663 | NZ | LYS | A | 460 | 6.879 | 20.456 | 11.191 | 1.00 | 35.01 | A | N |
| ATOM | 667 | C | LYS | A | 460 | 1.301 | 24.390 | 11.917 | 1.00 | 25.65 | A | C |
| ATOM | 668 | O | LYS | A | 460 | 0.444 | 23.818 | 11.226 | 1.00 | 25.83 | A | O |
| ATOM | 669 | N | LYS | A | 461 | 1.574 | 25.687 | 11.808 | 1.00 | 25.80 | A | N |
| ATOM | 671 | CA | LYS | A | 461 | 0.826 | 26.538 | 10.882 | 1.00 | 26.62 | A | C |
| ATOM | 673 | CB | LYS | A | 461 | 1.252 | 28.006 | 11.008 | 1.00 | 26.88 | A | C |
| ATOM | 676 | CG | LYS | A | 461 | 2.713 | 28.253 | 10.821 | 1.00 | 27.10 | A | C |
| ATOM | 679 | CD | LYS | A | 461 | 2.998 | 29.749 | 10.885 | 1.00 | 26.75 | A | C |
| ATOM | 682 | CE | LYS | A | 461 | 4.299 | 30.069 | 10.215 | 1.00 | 26.14 | A | C |
| ATOM | 685 | NZ | LYS | A | 461 | 4.520 | 31.513 | 10.213 | 1.00 | 24.09 | A | N |
| ATOM | 689 | C | LYS | A | 461 | 0.920 | 26.065 | 9.424 | 1.00 | 26.93 | A | C |
| ATOM | 690 | O | LYS | A | 461 | −0.036 | 26.221 | 8.675 | 1.00 | 27.53 | A | O |
| ATOM | 691 | N | ASP | A | 462 | 2.046 | 25.462 | 9.044 | 1.00 | 27.62 | A | N |
| ATOM | 693 | CA | ASP | A | 462 | 2.260 | 24.961 | 7.678 | 1.00 | 28.08 | A | C |
| ATOM | 695 | CB | ASP | A | 462 | 3.747 | 25.062 | 7.309 | 1.00 | 28.77 | A | C |
| ATOM | 698 | CG | ASP | A | 462 | 4.025 | 24.701 | 5.853 | 1.00 | 30.67 | A | C |
| ATOM | 699 | OD1 | ASP | A | 462 | 3.273 | 25.156 | 4.964 | 1.00 | 33.53 | A | O |
| ATOM | 700 | OD2 | ASP | A | 462 | 4.973 | 23.959 | 5.506 | 1.00 | 34.82 | A | O |
| ATOM | 701 | C | ASP | A | 462 | 1.772 | 23.509 | 7.560 | 1.00 | 27.68 | A | C |
| ATOM | 702 | O | ASP | A | 462 | 2.542 | 22.601 | 7.211 | 1.00 | 28.33 | A | O |
| ATOM | 703 | N | CYS | A | 463 | 0.500 | 23.302 | 7.891 | 1.00 | 26.54 | A | N |
| ATOM | 705 | CA | CYS | A | 463 | −0.123 | 21.991 | 7.824 | 1.00 | 25.64 | A | C |
| ATOM | 707 | CB | CYS | A | 463 | −0.529 | 21.522 | 9.217 | 1.00 | 25.87 | A | C |
| ATOM | 710 | SG | CYS | A | 463 | −1.141 | 19.833 | 9.252 | 1.00 | 28.94 | A | S |
| ATOM | 711 | C | CYS | A | 463 | −1.350 | 22.104 | 6.918 | 1.00 | 23.92 | A | C |
| ATOM | 712 | O | CYS | A | 463 | −2.319 | 22.772 | 7.251 | 1.00 | 23.05 | A | O |
| ATOM | 713 | N | THR | A | 464 | −1.277 | 21.480 | 5.751 | 1.00 | 22.41 | A | N |
| ATOM | 715 | CA | THR | A | 464 | −2.385 | 21.500 | 4.800 | 1.00 | 21.51 | A | C |
| ATOM | 717 | CB | THR | A | 464 | −1.947 | 20.928 | 3.435 | 1.00 | 21.47 | A | C |
| ATOM | 719 | OG1 | THR | A | 464 | −1.405 | 19.607 | 3.600 | 1.00 | 19.22 | A | O |
| ATOM | 721 | CG2 | THR | A | 464 | −0.804 | 21.743 | 2.823 | 1.00 | 21.55 | A | C |
| ATOM | 725 | C | THR | A | 464 | −3.571 | 20.689 | 5.316 | 1.00 | 21.41 | A | C |
| ATOM | 726 | O | THR | A | 464 | −3.414 | 19.755 | 6.111 | 1.00 | 19.82 | A | O |
| ATOM | 727 | N | LEU | A | 465 | −4.758 | 21.040 | 4.832 | 1.00 | 21.68 | A | N |
| ATOM | 729 | CA | LEU | A | 465 | −5.969 | 20.261 | 5.090 | 1.00 | 22.10 | A | C |
| ATOM | 731 | CB | LEU | A | 465 | −7.165 | 20.906 | 4.378 | 1.00 | 22.26 | A | C |
| ATOM | 734 | CG | LEU | A | 465 | −7.631 | 22.229 | 4.986 | 1.00 | 22.45 | A | C |
| ATOM | 736 | CD1 | LEU | A | 465 | −8.763 | 22.808 | 4.170 | 1.00 | 24.00 | A | C |
| ATOM | 740 | CD2 | LEU | A | 465 | −8.079 | 22.028 | 6.446 | 1.00 | 23.54 | A | C |
| ATOM | 744 | C | LEU | A | 465 | −5.798 | 18.821 | 4.624 | 1.00 | 22.80 | A | C |
| ATOM | 745 | O | LEU | A | 465 | −6.322 | 17.892 | 5.234 | 1.00 | 22.69 | A | O |
| ATOM | 746 | N | ASP | A | 466 | −5.073 | 18.665 | 3.524 | 1.00 | 23.58 | A | N |
| ATOM | 748 | CA | ASP | A | 466 | −4.666 | 17.367 | 3.004 | 1.00 | 25.03 | A | C |
| ATOM | 750 | CB | ASP | A | 466 | −3.709 | 17.593 | 1.820 | 1.00 | 25.18 | A | C |
| ATOM | 753 | CG | ASP | A | 466 | −3.414 | 16.332 | 1.038 | 1.00 | 27.16 | A | C |
| ATOM | 754 | OD1 | ASP | A | 466 | −3.623 | 15.216 | 1.570 | 1.00 | 26.35 | A | O |
| ATOM | 755 | OD2 | ASP | A | 466 | −2.966 | 16.376 | −0.139 | 1.00 | 29.99 | A | O |
| ATOM | 756 | C | ASP | A | 466 | −3.993 | 16.539 | 4.101 | 1.00 | 25.88 | A | C |
| ATOM | 757 | O | ASP | A | 466 | −4.460 | 15.448 | 4.445 | 1.00 | 25.28 | A | O |
| ATOM | 758 | N | ASN | A | 467 | −2.900 | 17.063 | 4.656 | 1.00 | 27.10 | A | N |
| ATOM | 760 | CA | ASN | A | 467 | −2.161 | 16.350 | 5.702 | 1.00 | 28.05 | A | C |
| ATOM | 762 | CB | ASN | A | 467 | −0.772 | 16.985 | 5.916 | 1.00 | 28.48 | A | C |
| ATOM | 765 | CG | ASN | A | 467 | 0.134 | 16.866 | 4.684 | 1.00 | 29.52 | A | C |
| ATOM | 766 | OD1 | ASN | A | 467 | 0.992 | 17.713 | 4.447 | 1.00 | 31.12 | A | O |
| ATOM | 767 | ND2 | ASN | A | 467 | −0.059 | 15.816 | 3.901 | 1.00 | 33.05 | A | N |
| ATOM | 770 | C | ASN | A | 467 | −2.920 | 16.263 | 7.037 | 1.00 | 28.75 | A | C |
| ATOM | 771 | O | ASN | A | 467 | −2.724 | 15.322 | 7.806 | 1.00 | 28.72 | A | O |
| ATOM | 772 | N | LYS | A | 468 | −3.794 | 17.232 | 7.296 | 1.00 | 29.65 | A | N |
| ATOM | 774 | CA | LYS | A | 468 | −4.560 | 17.300 | 8.544 | 1.00 | 30.66 | A | C |
| ATOM | 776 | CB | LYS | A | 468 | −5.273 | 18.642 | 8.639 | 1.00 | 30.86 | A | C |
| ATOM | 779 | CG | LYS | A | 468 | −5.702 | 19.038 | 10.031 | 1.00 | 32.87 | A | C |
| ATOM | 782 | CD | LYS | A | 468 | −4.950 | 20.278 | 10.513 | 1.00 | 35.25 | A | C |
| ATOM | 785 | CE | LYS | A | 468 | −5.304 | 21.521 | 9.671 | 1.00 | 36.51 | A | C |
| ATOM | 788 | NZ | LYS | A | 468 | −4.860 | 22.805 | 10.300 | 1.00 | 37.11 | A | N |
| ATOM | 792 | C | LYS | A | 468 | −5.604 | 16.186 | 8.644 | 1.00 | 31.56 | A | C |
| ATOM | 793 | O | LYS | A | 468 | −5.905 | 15.716 | 9.740 | 1.00 | 31.30 | A | O |
| ATOM | 794 | N | GLU | A | 469 | −6.159 | 15.779 | 7.504 | 1.00 | 32.55 | A | N |
| ATOM | 796 | CA | GLU | A | 469 | −7.144 | 14.694 | 7.473 | 1.00 | 33.52 | A | C |
| ATOM | 798 | CB | GLU | A | 469 | −7.866 | 14.646 | 6.117 | 1.00 | 33.78 | A | C |
| ATOM | 801 | CG | GLU | A | 469 | −8.835 | 13.476 | 5.924 | 1.00 | 34.57 | A | C |
| ATOM | 804 | CD | GLU | A | 469 | −9.939 | 13.398 | 6.975 | 1.00 | 36.41 | A | C |
| ATOM | 805 | OE1 | GLU | A | 469 | −10.241 | 14.421 | 7.638 | 1.00 | 36.96 | A | O |
| ATOM | 806 | OE2 | GLU | A | 469 | −10.527 | 12.301 | 7.131 | 1.00 | 38.63 | A | O |
| ATOM | 807 | C | GLU | A | 469 | −6.471 | 13.355 | 7.789 | 1.00 | 34.36 | A | C |
| ATOM | 808 | O | GLU | A | 469 | −7.067 | 12.508 | 8.436 | 1.00 | 33.99 | A | O |
| ATOM | 809 | N | LYS | A | 470 | −5.226 | 13.185 | 7.344 | 1.00 | 35.36 | A | N |
| ATOM | 811 | CA | LYS | A | 470 | −4.441 | 11.983 | 7.651 | 1.00 | 36.27 | A | C |
| ATOM | 813 | CB | LYS | A | 470 | −3.087 | 12.022 | 6.942 | 1.00 | 36.53 | A | C |
| ATOM | 816 | CG | LYS | A | 470 | −3.155 | 12.204 | 5.444 | 1.00 | 37.48 | A | C |
| ATOM | 819 | CD | LYS | A | 470 | −1.766 | 12.076 | 4.833 | 1.00 | 38.99 | A | C |
| ATOM | 822 | CE | LYS | A | 470 | −1.822 | 11.889 | 3.334 | 1.00 | 39.11 | A | C |
| ATOM | 825 | NZ | LYS | A | 470 | −0.566 | 11.286 | 2.828 | 1.00 | 39.24 | A | N |
| ATOM | 829 | C | LYS | A | 470 | −4.184 | 11.839 | 9.144 | 1.00 | 36.64 | A | C |
| ATOM | 830 | O | LYS | A | 470 | −4.342 | 10.758 | 9.709 | 1.00 | 37.23 | A | O |
| ATOM | 831 | N | PHE | A | 471 | −3.773 | 12.938 | 9.763 | 1.00 | 36.96 | A | N |
| ATOM | 833 | CA | PHE | A | 471 | −3.512 | 13.023 | 11.201 | 1.00 | 37.25 | A | C |
| ATOM | 835 | CB | PHE | A | 471 | −2.955 | 14.420 | 11.517 | 1.00 | 37.75 | A | C |
| ATOM | 838 | CG | PHE | A | 471 | −2.489 | 14.615 | 12.947 | 1.00 | 40.27 | A | C |
| ATOM | 839 | CD1 | PHE | A | 471 | −1.834 | 13.605 | 13.650 | 1.00 | 42.26 | A | C |
| ATOM | 841 | CE1 | PHE | A | 471 | −1.407 | 13.815 | 14.958 | 1.00 | 43.12 | A | C |
| ATOM | 843 | CZ | PHE | A | 471 | −1.611 | 15.056 | 15.572 | 1.00 | 43.43 | A | C |
| ATOM | 845 | CE2 | PHE | A | 471 | −2.243 | 16.066 | 14.883 | 1.00 | 42.92 | A | C |
| ATOM | 847 | CD2 | PHE | A | 471 | −2.680 | 15.847 | 13.578 | 1.00 | 42.50 | A | C |
| ATOM | 849 | C | PHE | A | 471 | −4.770 | 12.764 | 12.031 | 1.00 | 36.80 | A | C |
| ATOM | 850 | O | PHE | A | 471 | −4.725 | 12.035 | 13.017 | 1.00 | 36.85 | A | O |
| ATOM | 851 | N | MET | A | 472 | −5.885 | 13.369 | 11.630 | 1.00 | 36.07 | A | N |
| ATOM | 853 | CA | MET | A | 472 | −7.154 | 13.199 | 12.338 | 1.00 | 36.08 | A | C |
| ATOM | 855 | CB | MET | A | 472 | −8.246 | 14.103 | 11.761 | 1.00 | 36.21 | A | C |
| ATOM | 858 | CG | MET | A | 472 | −8.083 | 15.565 | 12.091 | 1.00 | 38.47 | A | C |
| ATOM | 861 | SD | MET | A | 472 | −7.642 | 15.859 | 13.819 | 1.00 | 42.21 | A | S |
| ATOM | 862 | CE | MET | A | 472 | −5.860 | 15.966 | 13.721 | 1.00 | 42.25 | A | C |
| ATOM | 866 | C | MET | A | 472 | −7.626 | 11.758 | 12.262 | 1.00 | 35.10 | A | C |
| ATOM | 867 | O | MET | A | 472 | −8.011 | 11.176 | 13.278 | 1.00 | 34.68 | A | O |
| ATOM | 868 | N | SER | A | 473 | −7.599 | 11.200 | 11.056 | 1.00 | 34.38 | A | N |
| ATOM | 870 | CA | SER | A | 473 | −8.067 | 9.836 | 10.838 | 1.00 | 34.10 | A | C |
| ATOM | 872 | CB | SER | A | 473 | −8.111 | 9.481 | 9.341 | 1.00 | 34.17 | A | C |
| ATOM | 875 | OG | SER | A | 473 | −6.876 | 9.725 | 8.700 | 1.00 | 35.90 | A | O |
| ATOM | 877 | C | SER | A | 473 | −7.194 | 8.870 | 11.619 | 1.00 | 33.23 | A | C |
| ATOM | 878 | O | SER | A | 473 | −7.681 | 7.877 | 12.146 | 1.00 | 33.23 | A | O |
| ATOM | 879 | N | GLU | A | 474 | −5.909 | 9.196 | 11.728 | 1.00 | 32.29 | A | N |
| ATOM | 881 | CA | GLU | A | 474 | −4.957 | 8.405 | 12.504 | 1.00 | 31.53 | A | C |
| ATOM | 883 | CB | GLU | A | 474 | −3.532 | 8.889 | 12.235 | 1.00 | 32.06 | A | C |
| ATOM | 886 | CG | GLU | A | 474 | −2.462 | 8.083 | 12.940 | 1.00 | 35.27 | A | C |
| ATOM | 889 | CD | GLU | A | 474 | −1.059 | 8.482 | 12.520 | 1.00 | 38.82 | A | C |
| ATOM | 890 | OE1 | GLU | A | 474 | −0.125 | 7.684 | 12.765 | 1.00 | 41.03 | A | O |
| ATOM | 891 | OE2 | GLU | A | 474 | −0.896 | 9.590 | 11.945 | 1.00 | 41.72 | A | O |
| ATOM | 892 | C | GLU | A | 474 | −5.252 | 8.474 | 13.999 | 1.00 | 29.52 | A | C |
| ATOM | 893 | O | GLU | A | 474 | −5.194 | 7.463 | 14.689 | 1.00 | 28.59 | A | O |
| ATOM | 894 | N | ALA | A | 475 | −5.583 | 9.664 | 14.486 | 1.00 | 27.61 | A | N |
| ATOM | 896 | CA | ALA | A | 475 | −5.852 | 9.876 | 15.908 | 1.00 | 26.39 | A | C |
| ATOM | 898 | CB | ALA | A | 475 | −5.991 | 11.361 | 16.196 | 1.00 | 26.28 | A | C |
| ATOM | 902 | C | ALA | A | 475 | −7.117 | 9.144 | 16.379 | 1.00 | 25.41 | A | C |
| ATOM | 903 | O | ALA | A | 475 | −7.186 | 8.717 | 17.521 | 1.00 | 24.68 | A | O |
| ATOM | 904 | N | VAL | A | 476 | −8.111 | 9.024 | 15.501 | 1.00 | 24.57 | A | N |
| ATOM | 906 | CA | VAL | A | 476 | −9.364 | 8.342 | 15.837 | 1.00 | 24.02 | A | C |
| ATOM | 908 | CB | VAL | A | 476 | −10.457 | 8.593 | 14.764 | 1.00 | 24.14 | A | C |
| ATOM | 910 | CG1 | VAL | A | 476 | −11.668 | 7.695 | 14.975 | 1.00 | 24.34 | A | C |
| ATOM | 914 | CG2 | VAL | A | 476 | −10.910 | 10.051 | 14.786 | 1.00 | 24.76 | A | C |
| ATOM | 918 | C | VAL | A | 476 | −9.096 | 6.840 | 16.010 | 1.00 | 23.50 | A | C |
| ATOM | 919 | O | VAL | A | 476 | −9.673 | 6.207 | 16.889 | 1.00 | 22.85 | A | O |
| ATOM | 920 | N | ILE | A | 477 | −8.191 | 6.283 | 15.197 | 1.00 | 23.32 | A | N |
| ATOM | 922 | CA | ILE | A | 477 | −7.794 | 4.880 | 15.366 | 1.00 | 23.29 | A | C |
| ATOM | 924 | CB | ILE | A | 477 | −6.857 | 4.392 | 14.232 | 1.00 | 23.54 | A | C |
| ATOM | 926 | CG1 | ILE | A | 477 | −7.579 | 4.420 | 12.883 | 1.00 | 25.55 | A | C |
| ATOM | 929 | CD1 | ILE | A | 477 | −6.693 | 4.070 | 11.676 | 1.00 | 26.07 | A | C |
| ATOM | 933 | CG2 | ILE | A | 477 | −6.357 | 2.978 | 14.550 | 1.00 | 24.90 | A | C |
| ATOM | 937 | C | ILE | A | 477 | −7.126 | 4.693 | 16.729 | 1.00 | 22.19 | A | C |
| ATOM | 938 | O | ILE | A | 477 | −7.505 | 3.833 | 17.502 | 1.00 | 20.75 | A | O |
| ATOM | 939 | N | MET | A | 478 | −6.126 | 5.518 | 17.025 | 1.00 | 22.05 | A | N |
| ATOM | 941 | CA | MET | A | 478 | −5.479 | 5.507 | 18.341 | 1.00 | 21.67 | A | C |
| ATOM | 943 | CB | MET | A | 478 | −4.401 | 6.603 | 18.421 | 1.00 | 22.21 | A | C |
| ATOM | 946 | CG | MET | A | 478 | −3.172 | 6.370 | 17.537 | 1.00 | 23.35 | A | C |
| ATOM | 949 | SD | MET | A | 478 | −2.324 | 4.761 | 17.784 | 1.00 | 27.04 | A | S |
| ATOM | 950 | CE | MET | A | 478 | −1.687 | 4.917 | 19.390 | 1.00 | 24.74 | A | C |
| ATOM | 954 | C | MET | A | 478 | −6.463 | 5.660 | 19.508 | 1.00 | 21.22 | A | C |
| ATOM | 955 | O | MET | A | 478 | −6.280 | 5.042 | 20.541 | 1.00 | 20.80 | A | O |
| ATOM | 956 | N | LYS | A | 479 | −7.517 | 6.461 | 19.336 | 1.00 | 21.22 | A | N |
| ATOM | 958 | CA | LYS | A | 479 | −8.521 | 6.681 | 20.381 | 1.00 | 21.07 | A | C |
| ATOM | 960 | CB | LYS | A | 479 | −9.550 | 7.727 | 19.910 | 1.00 | 21.83 | A | C |
| ATOM | 963 | CG | LYS | A | 479 | −10.728 | 7.940 | 20.864 | 1.00 | 25.29 | A | C |
| ATOM | 966 | CD | LYS | A | 479 | −11.616 | 9.074 | 20.401 | 1.00 | 28.79 | A | C |
| ATOM | 969 | CE | LYS | A | 479 | −12.585 | 9.512 | 21.494 | 1.00 | 30.50 | A | C |
| ATOM | 972 | NZ | LYS | A | 479 | −13.545 | 8.454 | 21.868 | 1.00 | 31.90 | A | N |
| ATOM | 976 | C | LYS | A | 479 | −9.251 | 5.394 | 20.752 | 1.00 | 20.32 | A | C |
| ATOM | 977 | O | LYS | A | 479 | −9.653 | 5.195 | 21.906 | 1.00 | 20.37 | A | O |
| ATOM | 978 | N | ASN | A | 480 | −9.447 | 4.529 | 19.761 | 1.00 | 18.51 | A | N |
| ATOM | 980 | CA | ASN | A | 480 | −10.168 | 3.284 | 19.980 | 1.00 | 18.01 | A | C |
| ATOM | 982 | CB | ASN | A | 480 | −10.851 | 2.841 | 18.692 | 1.00 | 17.94 | A | C |
| ATOM | 985 | CG | ASN | A | 480 | −12.098 | 3.625 | 18.410 | 1.00 | 16.43 | A | C |
| ATOM | 986 | OD1 | ASN | A | 480 | −13.135 | 3.388 | 19.038 | 1.00 | 14.27 | A | O |
| ATOM | 987 | ND2 | ASN | A | 480 | −12.016 | 4.574 | 17.481 | 1.00 | 13.64 | A | N |
| ATOM | 990 | C | ASN | A | 480 | −9.277 | 2.174 | 20.512 | 1.00 | 17.93 | A | C |
| ATOM | 991 | O | ASN | A | 480 | −9.768 | 1.196 | 21.049 | 1.00 | 17.90 | A | O |
| ATOM | 992 | N | LEU | A | 481 | −7.969 | 2.305 | 20.345 | 1.00 | 18.54 | A | N |
| ATOM | 994 | CA | LEU | A | 481 | −7.052 | 1.344 | 20.933 | 1.00 | 19.58 | A | C |
| ATOM | 996 | CB | LEU | A | 481 | −5.647 | 1.510 | 20.375 | 1.00 | 19.65 | A | C |
| ATOM | 999 | CG | LEU | A | 481 | −5.418 | 0.995 | 18.958 | 1.00 | 19.49 | A | C |
| ATOM | 1001 | CD1 | LEU | A | 481 | −4.028 | 1.362 | 18.492 | 1.00 | 20.75 | A | C |
| ATOM | 1005 | CD2 | LEU | A | 481 | −5.645 | −0.497 | 18.891 | 1.00 | 19.12 | A | C |
| ATOM | 1009 | C | LEU | A | 481 | −7.028 | 1.560 | 22.430 | 1.00 | 20.27 | A | C |
| ATOM | 1010 | O | LEU | A | 481 | −6.658 | 2.630 | 22.899 | 1.00 | 22.42 | A | O |
| ATOM | 1011 | N | ASP | A | 482 | −7.411 | 0.543 | 23.177 | 1.00 | 20.28 | A | N |
| ATOM | 1013 | CA | ASP | A | 482 | −7.360 | 0.583 | 24.626 | 1.00 | 20.20 | A | C |
| ATOM | 1015 | CB | ASP | A | 482 | −8.781 | 0.626 | 25.196 | 1.00 | 21.36 | A | C |
| ATOM | 1018 | CG | ASP | A | 482 | −8.815 | 0.831 | 26.702 | 1.00 | 24.17 | A | C |
| ATOM | 1019 | OD1 | ASP | A | 482 | −9.901 | 0.603 | 27.288 | 1.00 | 31.84 | A | O |
| ATOM | 1020 | OD2 | ASP | A | 482 | −7.843 | 1.228 | 27.382 | 1.00 | 28.34 | A | O |
| ATOM | 1021 | C | ASP | A | 482 | −6.625 | −0.664 | 25.080 | 1.00 | 19.09 | A | C |
| ATOM | 1022 | O | ASP | A | 482 | −7.178 | −1.765 | 25.073 | 1.00 | 19.88 | A | O |
| ATOM | 1023 | N | HIS | A | 483 | −5.349 | −0.490 | 25.413 | 1.00 | 16.36 | A | N |
| ATOM | 1025 | CA | HIS | A | 483 | −4.508 | −1.572 | 25.890 | 1.00 | 15.18 | A | C |
| ATOM | 1027 | CB | HIS | A | 483 | −3.723 | −2.178 | 24.727 | 1.00 | 14.01 | A | C |
| ATOM | 1030 | CG | HIS | A | 483 | −3.068 | −3.474 | 25.069 | 1.00 | 13.88 | A | C |
| ATOM | 1031 | ND1 | HIS | A | 483 | −1.872 | −3.542 | 25.755 | 1.00 | 13.01 | A | N |
| ATOM | 1033 | CE1 | HIS | A | 483 | −1.569 | −4.812 | 25.969 | 1.00 | 13.34 | A | C |
| ATOM | 1035 | NE2 | HIS | A | 483 | −2.515 | −5.566 | 25.429 | 1.00 | 12.59 | A | N |
| ATOM | 1037 | CD2 | HIS | A | 483 | −3.465 | −4.752 | 24.867 | 1.00 | 12.02 | A | C |
| ATOM | 1039 | C | HIS | A | 483 | −3.553 | −1.015 | 26.964 | 1.00 | 14.57 | A | C |
| ATOM | 1040 | O | HIS | A | 483 | −3.092 | 0.118 | 26.840 | 1.00 | 14.09 | A | O |
| ATOM | 1041 | N | PRO | A | 484 | −3.278 | −1.775 | 28.023 | 1.00 | 14.32 | A | N |
| ATOM | 1042 | CA | PRO | A | 484 | −2.360 | −1.310 | 29.080 | 1.00 | 13.52 | A | C |
| ATOM | 1044 | CB | PRO | A | 484 | −2.254 | −2.519 | 30.037 | 1.00 | 14.18 | A | C |
| ATOM | 1047 | CG | PRO | A | 484 | −3.355 | −3.429 | 29.700 | 1.00 | 15.62 | A | C |
| ATOM | 1050 | CD | PRO | A | 484 | −3.866 | −3.091 | 28.339 | 1.00 | 14.94 | A | C |
| ATOM | 1053 | C | PRO | A | 484 | −0.956 | −0.924 | 28.610 | 1.00 | 12.37 | A | C |
| ATOM | 1054 | O | PRO | A | 484 | −0.285 | −0.173 | 29.308 | 1.00 | 12.09 | A | O |
| ATOM | 1055 | N | HIS | A | 485 | −0.532 | −1.423 | 27.448 | 1.00 | 11.71 | A | N |
| ATOM | 1057 | CA | HIS | A | 485 | 0.790 | −1.148 | 26.915 | 1.00 | 10.90 | A | C |
| ATOM | 1059 | CB | HIS | A | 485 | 1.606 | −2.438 | 26.948 | 1.00 | 11.48 | A | C |
| ATOM | 1062 | CG | HIS | A | 485 | 1.687 | −2.999 | 28.326 | 1.00 | 11.67 | A | C |
| ATOM | 1063 | ND1 | HIS | A | 485 | 2.263 | −2.293 | 29.357 | 1.00 | 10.00 | A | N |
| ATOM | 1065 | CE1 | HIS | A | 485 | 2.139 | −2.984 | 30.478 | 1.00 | 13.83 | A | C |
| ATOM | 1067 | NE2 | HIS | A | 485 | 1.496 | −4.105 | 30.211 | 1.00 | 13.41 | A | N |
| ATOM | 1069 | CD2 | HIS | A | 485 | 1.179 | −4.129 | 28.873 | 1.00 | 13.59 | A | C |
| ATOM | 1071 | C | HIS | A | 485 | 0.786 | −0.514 | 25.549 | 1.00 | 10.49 | A | C |
| ATOM | 1072 | O | HIS | A | 485 | 1.722 | −0.706 | 24.795 | 1.00 | 9.78 | A | O |
| ATOM | 1073 | N | ILE | A | 486 | −0.267 | 0.258 | 25.260 | 1.00 | 9.75 | A | N |
| ATOM | 1075 | CA | ILE | A | 486 | −0.313 | 1.165 | 24.107 | 1.00 | 9.72 | A | C |
| ATOM | 1077 | CB | ILE | A | 486 | −1.352 | 0.675 | 23.077 | 1.00 | 9.49 | A | C |
| ATOM | 1079 | CG1 | ILE | A | 486 | −0.941 | −0.698 | 22.509 | 1.00 | 9.88 | A | C |
| ATOM | 1082 | CD1 | ILE | A | 486 | −1.944 | −1.282 | 21.569 | 1.00 | 11.12 | A | C |
| ATOM | 1086 | CG2 | ILE | A | 486 | −1.516 | 1.670 | 21.927 | 1.00 | 10.54 | A | C |
| ATOM | 1090 | C | ILE | A | 486 | −0.683 | 2.561 | 24.617 | 1.00 | 10.03 | A | C |
| ATOM | 1091 | O | ILE | A | 486 | −1.500 | 2.684 | 25.533 | 1.00 | 9.12 | A | O |
| ATOM | 1092 | N | VAL | A | 487 | −0.079 | 3.585 | 24.034 | 1.00 | 10.75 | A | N |
| ATOM | 1094 | CA | VAL | A | 487 | −0.294 | 4.958 | 24.477 | 1.00 | 11.70 | A | C |
| ATOM | 1096 | CB | VAL | A | 487 | 0.535 | 6.007 | 23.674 | 1.00 | 11.46 | A | C |
| ATOM | 1098 | CG1 | VAL | A | 487 | 2.006 | 5.849 | 23.946 | 1.00 | 11.31 | A | C |
| ATOM | 1102 | CG2 | VAL | A | 487 | 0.242 | 5.925 | 22.175 | 1.00 | 12.24 | A | C |
| ATOM | 1106 | C | VAL | A | 487 | −1.782 | 5.278 | 24.394 | 1.00 | 12.42 | A | C |
| ATOM | 1107 | O | VAL | A | 487 | −2.479 | 4.824 | 23.481 | 1.00 | 10.87 | A | O |
| ATOM | 1108 | N | LYS | A | 488 | −2.269 | 6.010 | 25.386 | 1.00 | 14.04 | A | N |
| ATOM | 1110 | CA | LYS | A | 488 | −3.699 | 6.252 | 25.531 | 1.00 | 15.40 | A | C |
| ATOM | 1112 | CB | LYS | A | 488 | −4.147 | 6.081 | 26.979 | 1.00 | 16.29 | A | C |
| ATOM | 1115 | CG | LYS | A | 488 | −5.648 | 6.355 | 27.161 | 1.00 | 18.92 | A | C |
| ATOM | 1118 | CD | LYS | A | 488 | −6.109 | 6.153 | 28.588 | 1.00 | 22.97 | A | C |
| ATOM | 1121 | CE | LYS | A | 488 | −7.638 | 6.269 | 28.693 | 1.00 | 26.06 | A | C |
| ATOM | 1124 | NZ | LYS | A | 488 | −8.179 | 5.734 | 29.994 | 1.00 | 28.52 | A | N |
| ATOM | 1128 | C | LYS | A | 488 | −4.019 | 7.663 | 25.090 | 1.00 | 16.61 | A | C |
| ATOM | 1129 | O | LYS | A | 488 | −3.470 | 8.625 | 25.650 | 1.00 | 15.54 | A | O |
| ATOM | 1130 | N | LEU | A | 489 | −4.879 | 7.760 | 24.079 | 1.00 | 18.15 | A | N |
| ATOM | 1132 | CA | LEU | A | 489 | −5.458 | 9.028 | 23.644 | 1.00 | 20.47 | A | C |
| ATOM | 1134 | CB | LEU | A | 489 | −5.707 | 9.035 | 22.135 | 1.00 | 20.96 | A | C |
| ATOM | 1137 | CG | LEU | A | 489 | −6.318 | 10.312 | 21.519 | 1.00 | 22.08 | A | C |
| ATOM | 1139 | CD1 | LEU | A | 489 | −6.157 | 10.316 | 20.034 | 1.00 | 23.52 | A | C |
| ATOM | 1143 | CD2 | LEU | A | 489 | −7.776 | 10.472 | 21.876 | 1.00 | 24.93 | A | C |
| ATOM | 1147 | C | LEU | A | 489 | −6.734 | 9.229 | 24.426 | 1.00 | 22.11 | A | C |
| ATOM | 1148 | O | LEU | A | 489 | −7.625 | 8.370 | 24.402 | 1.00 | 23.38 | A | O |
| ATOM | 1149 | N | ILE | A | 490 | −6.813 | 10.361 | 25.125 | 1.00 | 22.70 | A | N |
| ATOM | 1151 | CA | ILE | A | 490 | −7.920 | 10.685 | 26.016 | 1.00 | 23.88 | A | C |
| ATOM | 1153 | CB | ILE | A | 490 | −7.400 | 11.539 | 27.185 | 1.00 | 24.15 | A | C |
| ATOM | 1155 | CG1 | ILE | A | 490 | −6.437 | 10.714 | 28.049 | 1.00 | 25.63 | A | C |
| ATOM | 1158 | CD1 | ILE | A | 490 | −5.849 | 11.484 | 29.192 | 1.00 | 25.52 | A | C |
| ATOM | 1162 | CG2 | ILE | A | 490 | −8.558 | 12.096 | 28.022 | 1.00 | 25.12 | A | C |
| ATOM | 1166 | C | ILE | A | 490 | −9.047 | 11.420 | 25.274 | 1.00 | 23.99 | A | C |
| ATOM | 1167 | O | ILE | A | 490 | −10.232 | 11.098 | 25.448 | 1.00 | 24.52 | A | O |
| ATOM | 1168 | N | GLY | A | 491 | −8.685 | 12.413 | 24.468 | 1.00 | 23.22 | A | N |
| ATOM | 1170 | CA | GLY | A | 491 | −9.677 | 13.139 | 23.681 | 1.00 | 23.44 | A | C |
| ATOM | 1173 | C | GLY | A | 491 | −9.145 | 13.889 | 22.481 | 1.00 | 23.06 | A | C |
| ATOM | 1174 | O | GLY | A | 491 | −7.944 | 14.066 | 22.320 | 1.00 | 21.24 | A | O |
| ATOM | 1175 | N | ILE | A | 492 | −10.069 | 14.324 | 21.623 | 1.00 | 23.34 | A | N |
| ATOM | 1177 | CA | ILE | A | 492 | −9.747 | 15.175 | 20.490 | 1.00 | 23.94 | A | C |
| ATOM | 1179 | CB | ILE | A | 492 | −9.914 | 14.394 | 19.167 | 1.00 | 24.29 | A | C |
| ATOM | 1181 | CG1 | ILE | A | 492 | −9.044 | 13.128 | 19.175 | 1.00 | 24.89 | A | C |
| ATOM | 1184 | CD1 | ILE | A | 492 | −9.389 | 12.139 | 18.099 | 1.00 | 26.68 | A | C |
| ATOM | 1188 | CG2 | ILE | A | 492 | −9.539 | 15.252 | 17.986 | 1.00 | 24.59 | A | C |
| ATOM | 1192 | C | ILE | A | 492 | −10.675 | 16.401 | 20.528 | 1.00 | 24.76 | A | C |
| ATOM | 1193 | O | ILE | A | 492 | −11.891 | 16.254 | 20.639 | 1.00 | 23.89 | A | O |
| ATOM | 1194 | N | ILE | A | 493 | −10.086 | 17.598 | 20.508 | 1.00 | 25.52 | A | N |
| ATOM | 1196 | CA | ILE | A | 493 | −10.828 | 18.833 | 20.257 | 1.00 | 26.54 | A | C |
| ATOM | 1198 | CB | ILE | A | 493 | −10.315 | 20.009 | 21.138 | 1.00 | 26.60 | A | C |
| ATOM | 1200 | CG1 | ILE | A | 493 | −10.215 | 19.614 | 22.613 | 1.00 | 26.82 | A | C |
| ATOM | 1203 | CD1 | ILE | A | 493 | −11.446 | 18.990 | 23.180 | 1.00 | 27.58 | A | C |
| ATOM | 1207 | CG2 | ILE | A | 493 | −11.217 | 21.249 | 20.961 | 1.00 | 26.95 | A | C |
| ATOM | 1211 | C | ILE | A | 493 | −10.609 | 19.151 | 18.785 | 1.00 | 27.17 | A | C |
| ATOM | 1212 | O | ILE | A | 493 | −9.531 | 19.576 | 18.404 | 1.00 | 26.34 | A | O |
| ATOM | 1213 | N | GLU | A | 494 | −11.628 | 18.927 | 17.959 | 1.00 | 28.32 | A | N |
| ATOM | 1215 | CA | GLU | A | 494 | −11.502 | 19.096 | 16.510 | 1.00 | 29.45 | A | C |
| ATOM | 1217 | CB | GLU | A | 494 | −12.698 | 18.444 | 15.800 | 1.00 | 30.09 | A | C |
| ATOM | 1220 | CG | GLU | A | 494 | −12.800 | 16.943 | 16.024 | 1.00 | 32.11 | A | C |
| ATOM | 1223 | CD | GLU | A | 494 | −13.958 | 16.292 | 15.280 | 1.00 | 35.78 | A | C |
| ATOM | 1224 | OE1 | GLU | A | 494 | −14.108 | 16.531 | 14.058 | 1.00 | 38.41 | A | O |
| ATOM | 1225 | OE2 | GLU | A | 494 | −14.717 | 15.524 | 15.918 | 1.00 | 38.33 | A | O |
| ATOM | 1226 | C | GLU | A | 494 | −11.390 | 20.567 | 16.098 | 1.00 | 29.72 | A | C |
| ATOM | 1227 | O | GLU | A | 494 | −10.667 | 20.900 | 15.160 | 1.00 | 29.12 | A | O |
| ATOM | 1228 | N | GLU | A | 495 | −12.073 | 21.432 | 16.846 | 1.00 | 30.47 | A | N |
| ATOM | 1230 | CA | GLU | A | 495 | −12.259 | 22.853 | 16.505 | 1.00 | 31.12 | A | C |
| ATOM | 1232 | CB | GLU | A | 495 | −13.324 | 23.465 | 17.435 | 1.00 | 31.66 | A | C |
| ATOM | 1235 | CG | GLU | A | 495 | −14.759 | 23.024 | 17.203 | 1.00 | 34.10 | A | C |
| ATOM | 1238 | CD | GLU | A | 495 | −14.995 | 21.541 | 17.444 | 1.00 | 36.85 | A | C |
| ATOM | 1239 | OE1 | GLU | A | 495 | −14.650 | 21.029 | 18.544 | 1.00 | 38.24 | A | O |
| ATOM | 1240 | OE2 | GLU | A | 495 | −15.521 | 20.885 | 16.517 | 1.00 | 39.02 | A | O |
| ATOM | 1241 | C | GLU | A | 495 | −10.978 | 23.695 | 16.633 | 1.00 | 30.92 | A | C |
| ATOM | 1242 | O | GLU | A | 495 | −9.950 | 23.203 | 17.113 | 1.00 | 30.08 | A | O |
| ATOM | 1243 | N | GLU | A | 496 | −11.084 | 24.966 | 16.207 | 1.00 | 30.74 | A | N |
| ATOM | 1245 | CA | GLU | A | 496 | −10.102 | 26.053 | 16.450 | 1.00 | 30.21 | A | C |
| ATOM | 1247 | CB | GLU | A | 496 | −10.331 | 26.722 | 17.809 | 1.00 | 30.71 | A | C |
| ATOM | 1250 | CG | GLU | A | 496 | −11.767 | 27.153 | 18.080 | 1.00 | 33.56 | A | C |
| ATOM | 1253 | CD | GLU | A | 496 | −12.218 | 28.297 | 17.176 | 1.00 | 37.40 | A | C |
| ATOM | 1254 | OE1 | GLU | A | 496 | −11.734 | 29.442 | 17.368 | 1.00 | 39.71 | A | O |
| ATOM | 1255 | OE2 | GLU | A | 496 | −13.057 | 28.053 | 16.273 | 1.00 | 39.53 | A | O |
| ATOM | 1256 | C | GLU | A | 496 | −8.688 | 25.536 | 16.214 | 1.00 | 28.69 | A | C |
| ATOM | 1257 | O | GLU | A | 496 | −8.518 | 24.890 | 15.181 | 1.00 | 29.82 | A | O |
| ATOM | 1258 | N | PRO | A | 497 | −7.673 | 25.765 | 17.068 | 1.00 | 26.40 | A | N |
| ATOM | 1259 | CA | PRO | A | 497 | −6.463 | 24.955 | 16.914 | 1.00 | 24.97 | A | C |
| ATOM | 1261 | CB | PRO | A | 497 | −5.435 | 25.650 | 17.812 | 1.00 | 25.01 | A | C |
| ATOM | 1264 | CG | PRO | A | 497 | −6.217 | 26.352 | 18.819 | 1.00 | 25.48 | A | C |
| ATOM | 1267 | CD | PRO | A | 497 | −7.508 | 26.726 | 18.176 | 1.00 | 26.20 | A | C |
| ATOM | 1270 | C | PRO | A | 497 | −6.785 | 23.545 | 17.400 | 1.00 | 23.59 | A | C |
| ATOM | 1271 | O | PRO | A | 497 | −7.238 | 23.365 | 18.525 | 1.00 | 21.60 | A | O |
| ATOM | 1272 | N | THR | A | 498 | −6.593 | 22.562 | 16.533 | 1.00 | 22.37 | A | N |
| ATOM | 1274 | CA | THR | A | 498 | −6.969 | 21.193 | 16.855 | 1.00 | 22.15 | A | C |
| ATOM | 1276 | CB | THR | A | 498 | −6.918 | 20.363 | 15.580 | 1.00 | 22.32 | A | C |
| ATOM | 1278 | OG1 | THR | A | 498 | −7.958 | 20.826 | 14.700 | 1.00 | 25.57 | A | O |
| ATOM | 1280 | CG2 | THR | A | 498 | −7.252 | 18.924 | 15.840 | 1.00 | 22.92 | A | C |
| ATOM | 1284 | C | THR | A | 498 | −6.022 | 20.654 | 17.922 | 1.00 | 20.09 | A | C |
| ATOM | 1285 | O | THR | A | 498 | −4.835 | 20.874 | 17.821 | 1.00 | 19.92 | A | O |
| ATOM | 1286 | N | TRP | A | 499 | −6.568 | 20.002 | 18.950 | 1.00 | 19.00 | A | N |
| ATOM | 1288 | CA | TRP | A | 499 | −5.777 | 19.413 | 20.040 | 1.00 | 17.23 | A | C |
| ATOM | 1290 | CB | TRP | A | 499 | −6.188 | 20.010 | 21.393 | 1.00 | 17.26 | A | C |
| ATOM | 1293 | CG | TRP | A | 499 | −5.734 | 21.440 | 21.693 | 1.00 | 16.11 | A | C |
| ATOM | 1294 | CD1 | TRP | A | 499 | −5.098 | 22.301 | 20.851 | 1.00 | 16.58 | A | C |
| ATOM | 1296 | NE1 | TRP | A | 499 | −4.865 | 23.499 | 21.483 | 1.00 | 15.91 | A | N |
| ATOM | 1298 | CE2 | TRP | A | 499 | −5.342 | 23.431 | 22.760 | 1.00 | 13.91 | A | C |
| ATOM | 1299 | CD2 | TRP | A | 499 | −5.906 | 22.149 | 22.927 | 1.00 | 14.32 | A | C |
| ATOM | 1300 | CE3 | TRP | A | 499 | −6.474 | 21.825 | 24.167 | 1.00 | 14.41 | A | C |
| ATOM | 1302 | CZ3 | TRP | A | 499 | −6.466 | 22.789 | 25.187 | 1.00 | 15.48 | A | C |
| ATOM | 1304 | CH2 | TRP | A | 499 | −5.914 | 24.063 | 24.973 | 1.00 | 15.79 | A | C |
| ATOM | 1306 | CZ2 | TRP | A | 499 | −5.345 | 24.401 | 23.770 | 1.00 | 16.05 | A | C |
| ATOM | 1308 | C | TRP | A | 499 | −6.011 | 17.891 | 20.113 | 1.00 | 16.56 | A | C |
| ATOM | 1309 | O | TRP | A | 499 | −7.161 | 17.444 | 20.219 | 1.00 | 16.56 | A | O |
| ATOM | 1310 | N | ILE | A | 500 | −4.933 | 17.111 | 20.082 | 1.00 | 15.45 | A | N |
| ATOM | 1312 | CA | ILE | A | 500 | −4.987 | 15.696 | 20.427 | 1.00 | 15.58 | A | C |
| ATOM | 1314 | CB | ILE | A | 500 | −4.126 | 14.870 | 19.461 | 1.00 | 16.46 | A | C |
| ATOM | 1316 | CG1 | ILE | A | 500 | −4.423 | 15.233 | 17.994 | 1.00 | 17.06 | A | C |
| ATOM | 1319 | CD1 | ILE | A | 500 | −5.887 | 15.183 | 17.626 | 1.00 | 18.75 | A | C |
| ATOM | 1323 | CG2 | ILE | A | 500 | −4.288 | 13.390 | 19.744 | 1.00 | 18.35 | A | C |
| ATOM | 1327 | C | ILE | A | 500 | −4.467 | 15.555 | 21.863 | 1.00 | 14.87 | A | C |
| ATOM | 1328 | O | ILE | A | 500 | −3.323 | 15.879 | 22.141 | 1.00 | 14.70 | A | O |
| ATOM | 1329 | N | ILE | A | 501 | −5.318 | 15.096 | 22.765 | 1.00 | 13.73 | A | N |
| ATOM | 1331 | CA | ILE | A | 501 | −4.998 | 14.985 | 24.178 | 1.00 | 13.36 | A | C |
| ATOM | 1333 | CB | ILE | A | 501 | −6.190 | 15.451 | 25.034 | 1.00 | 12.71 | A | C |
| ATOM | 1335 | CG1 | ILE | A | 501 | −6.719 | 16.788 | 24.496 | 1.00 | 14.26 | A | C |
| ATOM | 1338 | CD1 | ILE | A | 501 | −7.978 | 17.256 | 25.129 | 1.00 | 15.07 | A | C |
| ATOM | 1342 | CG2 | ILE | A | 501 | −5.768 | 15.583 | 26.483 | 1.00 | 12.38 | A | C |
| ATOM | 1346 | C | ILE | A | 501 | −4.629 | 13.546 | 24.539 | 1.00 | 13.36 | A | C |
| ATOM | 1347 | O | ILE | A | 501 | −5.469 | 12.638 | 24.448 | 1.00 | 13.51 | A | O |
| ATOM | 1348 | N | MET | A | 502 | −3.372 | 13.359 | 24.933 | 1.00 | 13.48 | A | N |
| ATOM | 1350 | CA | MET | A | 502 | −2.836 | 12.061 | 25.353 | 1.00 | 13.96 | A | C |
| ATOM | 1352 | CB | MET | A | 502 | −1.550 | 11.754 | 24.585 | 1.00 | 14.71 | A | C |
| ATOM | 1355 | CG | MET | A | 502 | −1.634 | 11.957 | 23.074 | 1.00 | 18.44 | A | C |
| ATOM | 1358 | SD | MET | A | 502 | −2.691 | 10.736 | 22.262 | 1.00 | 25.96 | A | S |
| ATOM | 1359 | CE | MET | A | 502 | −1.705 | 9.251 | 22.464 | 1.00 | 24.95 | A | C |
| ATOM | 1363 | C | MET | A | 502 | −2.498 | 12.057 | 26.845 | 1.00 | 13.14 | A | C |
| ATOM | 1364 | O | MET | A | 502 | −2.200 | 13.092 | 27.436 | 1.00 | 11.75 | A | O |
| ATOM | 1365 | N | GLU | A | 503 | −2.501 | 10.879 | 27.458 | 1.00 | 12.97 | A | N |
| ATOM | 1367 | CA | GLU | A | 503 | −1.947 | 10.721 | 28.784 | 1.00 | 13.82 | A | C |
| ATOM | 1369 | CB | GLU | A | 503 | −2.136 | 9.258 | 29.215 | 1.00 | 15.08 | A | C |
| ATOM | 1372 | CG | GLU | A | 503 | −1.603 | 8.887 | 30.583 | 1.00 | 18.56 | A | C |
| ATOM | 1375 | CD | GLU | A | 503 | −2.097 | 7.516 | 31.024 | 1.00 | 23.89 | A | C |
| ATOM | 1376 | OE1 | GLU | A | 503 | −2.315 | 6.636 | 30.153 | 1.00 | 26.84 | A | O |
| ATOM | 1377 | OE2 | GLU | A | 503 | −2.272 | 7.314 | 32.243 | 1.00 | 29.32 | A | O |
| ATOM | 1378 | C | GLU | A | 503 | −0.457 | 11.112 | 28.747 | 1.00 | 13.19 | A | C |
| ATOM | 1379 | O | GLU | A | 503 | 0.230 | 10.778 | 27.787 | 1.00 | 13.15 | A | O |
| ATOM | 1380 | N | LEU | A | 504 | 0.020 | 11.836 | 29.760 | 1.00 | 12.59 | A | N |
| ATOM | 1382 | CA | LEU | A | 504 | 1.440 | 12.210 | 29.862 | 1.00 | 13.43 | A | C |
| ATOM | 1384 | CB | LEU | A | 504 | 1.635 | 13.449 | 30.746 | 1.00 | 13.57 | A | C |
| ATOM | 1387 | CG | LEU | A | 504 | 3.065 | 13.981 | 30.844 | 1.00 | 14.83 | A | C |
| ATOM | 1389 | CD1 | LEU | A | 504 | 3.440 | 14.719 | 29.551 | 1.00 | 15.70 | A | C |
| ATOM | 1393 | CD2 | LEU | A | 504 | 3.281 | 14.893 | 32.069 | 1.00 | 17.43 | A | C |
| ATOM | 1397 | C | LEU | A | 504 | 2.237 | 11.049 | 30.443 | 1.00 | 13.16 | A | C |
| ATOM | 1398 | O | LEU | A | 504 | 1.831 | 10.448 | 31.436 | 1.00 | 12.13 | A | O |
| ATOM | 1399 | N | TYR | A | 505 | 3.361 | 10.740 | 29.807 | 1.00 | 13.87 | A | N |
| ATOM | 1401 | CA | TYR | A | 505 | 4.261 | 9.682 | 30.247 | 1.00 | 14.37 | A | C |
| ATOM | 1403 | CB | TYR | A | 505 | 4.379 | 8.582 | 29.159 | 1.00 | 15.13 | A | C |
| ATOM | 1406 | CG | TYR | A | 505 | 3.034 | 7.948 | 28.815 | 1.00 | 14.57 | A | C |
| ATOM | 1407 | CD1 | TYR | A | 505 | 2.254 | 7.352 | 29.802 | 1.00 | 16.24 | A | C |
| ATOM | 1409 | CE1 | TYR | A | 505 | 0.996 | 6.804 | 29.506 | 1.00 | 15.83 | A | C |
| ATOM | 1411 | CZ | TYR | A | 505 | 0.517 | 6.853 | 28.221 | 1.00 | 13.11 | A | C |
| ATOM | 1412 | OH | TYR | A | 505 | −0.713 | 6.332 | 27.933 | 1.00 | 15.41 | A | O |
| ATOM | 1414 | CE2 | TYR | A | 505 | 1.255 | 7.455 | 27.229 | 1.00 | 13.32 | A | C |
| ATOM | 1416 | CD2 | TYR | A | 505 | 2.506 | 8.017 | 27.532 | 1.00 | 14.19 | A | C |
| ATOM | 1418 | C | TYR | A | 505 | 5.566 | 10.408 | 30.557 | 1.00 | 14.61 | A | C |
| ATOM | 1419 | O | TYR | A | 505 | 6.379 | 10.672 | 29.675 | 1.00 | 13.90 | A | O |
| ATOM | 1420 | N | PRO | A | 506 | 5.707 | 10.836 | 31.812 | 1.00 | 15.51 | A | N |
| ATOM | 1421 | CA | PRO | A | 506 | 6.699 | 11.840 | 32.188 | 1.00 | 15.79 | A | C |
| ATOM | 1423 | CB | PRO | A | 506 | 6.291 | 12.186 | 33.616 | 1.00 | 16.40 | A | C |
| ATOM | 1426 | CG | PRO | A | 506 | 5.692 | 10.962 | 34.115 | 1.00 | 16.38 | A | C |
| ATOM | 1429 | CD | PRO | A | 506 | 4.903 | 10.414 | 32.974 | 1.00 | 16.03 | A | C |
| ATOM | 1432 | C | PRO | A | 506 | 8.168 | 11.364 | 32.119 | 1.00 | 15.41 | A | C |
| ATOM | 1433 | O | PRO | A | 506 | 9.055 | 12.186 | 32.029 | 1.00 | 15.80 | A | O |
| ATOM | 1434 | N | TYR | A | 507 | 8.406 | 10.062 | 32.106 | 1.00 | 14.60 | A | N |
| ATOM | 1436 | CA | TYR | A | 507 | 9.767 | 9.548 | 31.946 | 1.00 | 13.73 | A | C |
| ATOM | 1438 | CB | TYR | A | 507 | 9.872 | 8.143 | 32.516 | 1.00 | 13.74 | A | C |
| ATOM | 1441 | CG | TYR | A | 507 | 9.659 | 8.071 | 33.997 | 1.00 | 14.65 | A | C |
| ATOM | 1442 | CD1 | TYR | A | 507 | 10.704 | 8.269 | 34.879 | 1.00 | 15.68 | A | C |
| ATOM | 1444 | CE1 | TYR | A | 507 | 10.505 | 8.191 | 36.268 | 1.00 | 17.32 | A | C |
| ATOM | 1446 | CZ | TYR | A | 507 | 9.241 | 7.931 | 36.754 | 1.00 | 16.75 | A | C |
| ATOM | 1447 | OH | TYR | A | 507 | 9.002 | 7.873 | 38.099 | 1.00 | 19.60 | A | O |
| ATOM | 1449 | CE2 | TYR | A | 507 | 8.198 | 7.737 | 35.895 | 1.00 | 15.81 | A | C |
| ATOM | 1451 | CD2 | TYR | A | 507 | 8.408 | 7.807 | 34.521 | 1.00 | 14.55 | A | C |
| ATOM | 1453 | C | TYR | A | 507 | 10.276 | 9.550 | 30.491 | 1.00 | 13.31 | A | C |
| ATOM | 1454 | O | TYR | A | 507 | 11.451 | 9.268 | 30.268 | 1.00 | 13.27 | A | O |
| ATOM | 1455 | N | GLY | A | 508 | 9.411 | 9.838 | 29.516 | 1.00 | 12.24 | A | N |
| ATOM | 1457 | CA | GLY | A | 508 | 9.834 | 10.058 | 28.142 | 1.00 | 12.21 | A | C |
| ATOM | 1460 | C | GLY | A | 508 | 10.112 | 8.784 | 27.362 | 1.00 | 11.49 | A | C |
| ATOM | 1461 | O | GLY | A | 508 | 9.628 | 7.729 | 27.727 | 1.00 | 10.44 | A | O |
| ATOM | 1462 | N | GLU | A | 509 | 10.885 | 8.896 | 26.281 | 1.00 | 11.21 | A | N |
| ATOM | 1464 | CA | GLU | A | 509 | 11.183 | 7.744 | 25.416 | 1.00 | 11.54 | A | C |
| ATOM | 1466 | CB | GLU | A | 509 | 11.913 | 8.194 | 24.156 | 1.00 | 12.66 | A | C |
| ATOM | 1469 | CG | GLU | A | 509 | 11.143 | 9.164 | 23.279 | 1.00 | 15.29 | A | C |
| ATOM | 1472 | CD | GLU | A | 509 | 11.973 | 9.695 | 22.119 | 1.00 | 18.15 | A | C |
| ATOM | 1473 | OE1 | GLU | A | 509 | 13.122 | 9.251 | 21.947 | 1.00 | 19.31 | A | O |
| ATOM | 1474 | OE2 | GLU | A | 509 | 11.472 | 10.588 | 21.393 | 1.00 | 21.32 | A | O |
| ATOM | 1475 | C | GLU | A | 509 | 12.038 | 6.666 | 26.095 | 1.00 | 10.37 | A | C |
| ATOM | 1476 | O | GLU | A | 509 | 12.953 | 6.974 | 26.847 | 1.00 | 10.56 | A | O |
| ATOM | 1477 | N | LEU | A | 510 | 11.742 | 5.399 | 25.791 | 1.00 | 9.52 | A | N |
| ATOM | 1479 | CA | LEU | A | 510 | 12.447 | 4.275 | 26.392 | 1.00 | 9.25 | A | C |
| ATOM | 1481 | CB | LEU | A | 510 | 11.768 | 2.956 | 25.990 | 1.00 | 9.16 | A | C |
| ATOM | 1484 | CG | LEU | A | 510 | 12.395 | 1.682 | 26.566 | 1.00 | 8.82 | A | C |
| ATOM | 1486 | CD1 | LEU | A | 510 | 12.453 | 1.737 | 28.081 | 1.00 | 9.86 | A | C |
| ATOM | 1490 | CD2 | LEU | A | 510 | 11.596 | 0.465 | 26.104 | 1.00 | 9.45 | A | C |
| ATOM | 1494 | C | LEU | A | 510 | 13.948 | 4.249 | 26.057 | 1.00 | 9.51 | A | C |
| ATOM | 1495 | O | LEU | A | 510 | 14.772 | 3.937 | 26.925 | 1.00 | 9.34 | A | O |
| ATOM | 1496 | N | GLY | A | 511 | 14.322 | 4.610 | 24.824 | 1.00 | 9.82 | A | N |
| ATOM | 1498 | CA | GLY | A | 511 | 15.729 | 4.549 | 24.433 | 1.00 | 10.57 | A | C |
| ATOM | 1501 | C | GLY | A | 511 | 16.585 | 5.425 | 25.340 | 1.00 | 10.99 | A | C |
| ATOM | 1502 | O | GLY | A | 511 | 17.566 | 4.964 | 25.934 | 1.00 | 11.11 | A | O |
| ATOM | 1503 | N | HIS | A | 512 | 16.178 | 6.672 | 25.492 | 1.00 | 11.55 | A | N |
| ATOM | 1505 | CA | HIS | A | 512 | 16.875 | 7.608 | 26.374 | 1.00 | 12.69 | A | C |
| ATOM | 1507 | CB | HIS | A | 512 | 16.319 | 9.011 | 26.174 | 1.00 | 13.88 | A | C |
| ATOM | 1510 | CG | HIS | A | 512 | 16.559 | 9.541 | 24.795 | 1.00 | 17.28 | A | C |
| ATOM | 1511 | ND1 | HIS | A | 512 | 17.678 | 9.214 | 24.061 | 1.00 | 22.03 | A | N |
| ATOM | 1513 | CE1 | HIS | A | 512 | 17.618 | 9.808 | 22.882 | 1.00 | 22.98 | A | C |
| ATOM | 1515 | NE2 | HIS | A | 512 | 16.508 | 10.518 | 22.831 | 1.00 | 21.48 | A | N |
| ATOM | 1517 | CD2 | HIS | A | 512 | 15.821 | 10.360 | 24.011 | 1.00 | 20.94 | A | C |
| ATOM | 1519 | C | HIS | A | 512 | 16.810 | 7.196 | 27.840 | 1.00 | 11.73 | A | C |
| ATOM | 1520 | O | HIS | A | 512 | 17.795 | 7.308 | 28.560 | 1.00 | 11.30 | A | O |
| ATOM | 1521 | N | TYR | A | 513 | 15.664 | 6.682 | 28.269 | 1.00 | 10.95 | A | N |
| ATOM | 1523 | CA | TYR | A | 513 | 15.472 | 6.184 | 29.630 | 1.00 | 10.24 | A | C |
| ATOM | 1525 | CB | TYR | A | 513 | 14.034 | 5.677 | 29.797 | 1.00 | 10.11 | A | C |
| ATOM | 1528 | CG | TYR | A | 513 | 13.693 | 5.094 | 31.169 | 1.00 | 9.46 | A | C |
| ATOM | 1529 | CD1 | TYR | A | 513 | 13.157 | 5.901 | 32.160 | 1.00 | 9.43 | A | C |
| ATOM | 1531 | CE1 | TYR | A | 513 | 12.800 | 5.391 | 33.393 | 1.00 | 9.59 | A | C |
| ATOM | 1533 | CZ | TYR | A | 513 | 12.988 | 4.068 | 33.671 | 1.00 | 9.14 | A | C |
| ATOM | 1534 | OH | TYR | A | 513 | 12.628 | 3.617 | 34.912 | 1.00 | 12.42 | A | O |
| ATOM | 1536 | CE2 | TYR | A | 513 | 13.533 | 3.214 | 32.708 | 1.00 | 8.34 | A | C |
| ATOM | 1538 | CD2 | TYR | A | 513 | 13.854 | 3.735 | 31.452 | 1.00 | 8.09 | A | C |
| ATOM | 1540 | C | TYR | A | 513 | 16.469 | 5.068 | 29.965 | 1.00 | 10.47 | A | C |
| ATOM | 1541 | O | TYR | A | 513 | 17.110 | 5.099 | 31.009 | 1.00 | 10.13 | A | O |
| ATOM | 1542 | N | LEU | A | 514 | 16.623 | 4.102 | 29.058 | 1.00 | 10.69 | A | N |
| ATOM | 1544 | CA | LEU | A | 514 | 17.596 | 3.021 | 29.239 | 1.00 | 11.38 | A | C |
| ATOM | 1546 | CB | LEU | A | 514 | 17.478 | 1.997 | 28.115 | 1.00 | 11.46 | A | C |
| ATOM | 1549 | CG | LEU | A | 514 | 16.163 | 1.198 | 28.035 | 1.00 | 12.98 | A | C |
| ATOM | 1551 | CD1 | LEU | A | 514 | 16.082 | 0.442 | 26.729 | 1.00 | 14.53 | A | C |
| ATOM | 1555 | CD2 | LEU | A | 514 | 16.010 | 0.260 | 29.218 | 1.00 | 12.92 | A | C |
| ATOM | 1559 | C | LEU | A | 514 | 19.027 | 3.556 | 29.300 | 1.00 | 12.00 | A | C |
| ATOM | 1560 | O | LEU | A | 514 | 19.839 | 3.080 | 30.090 | 1.00 | 12.23 | A | O |
| ATOM | 1561 | N | GLU | A | 515 | 19.330 | 4.518 | 28.450 | 1.00 | 12.66 | A | N |
| ATOM | 1563 | CA | GLU | A | 515 | 20.650 | 5.160 | 28.447 | 1.00 | 14.53 | A | C |
| ATOM | 1565 | CB | GLU | A | 515 | 20.742 | 6.212 | 27.346 | 1.00 | 15.14 | A | C |
| ATOM | 1568 | CG | GLU | A | 515 | 20.701 | 5.656 | 25.929 | 1.00 | 19.10 | A | C |
| ATOM | 1571 | CD | GLU | A | 515 | 20.531 | 6.744 | 24.868 | 1.00 | 24.40 | A | C |
| ATOM | 1572 | OE1 | GLU | A | 515 | 20.496 | 7.948 | 25.236 | 1.00 | 29.66 | A | O |
| ATOM | 1573 | OE2 | GLU | A | 515 | 20.433 | 6.395 | 23.665 | 1.00 | 28.24 | A | O |
| ATOM | 1574 | C | GLU | A | 515 | 20.954 | 5.812 | 29.795 | 1.00 | 15.17 | A | C |
| ATOM | 1575 | O | GLU | A | 515 | 22.046 | 5.601 | 30.347 | 1.00 | 15.52 | A | O |
| ATOM | 1576 | N | ARG | A | 516 | 19.987 | 6.576 | 30.317 | 1.00 | 15.35 | A | N |
| ATOM | 1578 | CA | ARG | A | 516 | 20.114 | 7.306 | 31.595 | 1.00 | 16.48 | A | C |
| ATOM | 1580 | CB | ARG | A | 516 | 18.842 | 8.134 | 31.882 | 1.00 | 17.00 | A | C |
| ATOM | 1583 | CG | ARG | A | 516 | 18.722 | 9.407 | 31.115 | 1.00 | 19.06 | A | C |
| ATOM | 1586 | CD | ARG | A | 516 | 17.815 | 10.406 | 31.760 | 1.00 | 18.75 | A | C |
| ATOM | 1589 | NE | ARG | A | 516 | 16.482 | 9.894 | 32.079 | 1.00 | 17.60 | A | N |
| ATOM | 1591 | CZ | ARG | A | 516 | 15.500 | 9.702 | 31.197 | 1.00 | 17.67 | A | C |
| ATOM | 1592 | NH1 | ARG | A | 516 | 15.669 | 9.969 | 29.909 | 1.00 | 17.37 | A | N |
| ATOM | 1595 | NH2 | ARG | A | 516 | 14.323 | 9.248 | 31.612 | 1.00 | 20.07 | A | N |
| ATOM | 1598 | C | ARG | A | 516 | 20.298 | 6.382 | 32.790 | 1.00 | 16.29 | A | C |
| ATOM | 1599 | O | ARG | A | 516 | 21.032 | 6.702 | 33.743 | 1.00 | 15.28 | A | O |
| ATOM | 1600 | N | ASN | A | 517 | 19.609 | 5.245 | 32.745 | 1.00 | 16.03 | A | N |
| ATOM | 1602 | CA | ASN | A | 517 | 19.387 | 4.432 | 33.927 | 1.00 | 15.86 | A | C |
| ATOM | 1604 | CB | ASN | A | 517 | 17.876 | 4.249 | 34.155 | 1.00 | 15.83 | A | C |
| ATOM | 1607 | CG | ASN | A | 517 | 17.161 | 5.562 | 34.430 | 1.00 | 16.01 | A | C |
| ATOM | 1608 | OD1 | ASN | A | 517 | 16.182 | 5.918 | 33.758 | 1.00 | 16.60 | A | O |
| ATOM | 1609 | ND2 | ASN | A | 517 | 17.671 | 6.316 | 35.390 | 1.00 | 15.03 | A | N |
| ATOM | 1612 | C | ASN | A | 517 | 20.108 | 3.092 | 33.863 | 1.00 | 16.06 | A | C |
| ATOM | 1613 | O | ASN | A | 517 | 19.900 | 2.238 | 34.715 | 1.00 | 15.77 | A | O |
| ATOM | 1614 | N | LYS | A | 518 | 20.980 | 2.928 | 32.870 | 1.00 | 16.16 | A | N |
| ATOM | 1616 | CA | LYS | A | 518 | 21.613 | 1.641 | 32.606 | 1.00 | 17.63 | A | C |
| ATOM | 1618 | CB | LYS | A | 518 | 22.643 | 1.784 | 31.474 | 1.00 | 17.77 | A | C |
| ATOM | 1621 | CG | LYS | A | 518 | 23.632 | 0.636 | 31.368 | 1.00 | 20.82 | A | C |
| ATOM | 1624 | CD | LYS | A | 518 | 24.421 | 0.686 | 30.063 | 1.00 | 24.15 | A | C |
| ATOM | 1627 | CE | LYS | A | 518 | 25.172 | 1.979 | 29.909 | 1.00 | 26.03 | A | C |
| ATOM | 1630 | NZ | LYS | A | 518 | 24.304 | 3.106 | 29.435 | 1.00 | 28.18 | A | N |
| ATOM | 1634 | C | LYS | A | 518 | 22.254 | 1.017 | 33.849 | 1.00 | 18.11 | A | C |
| ATOM | 1635 | O | LYS | A | 518 | 22.126 | −0.189 | 34.086 | 1.00 | 18.39 | A | O |
| ATOM | 1636 | N | ASN | A | 519 | 22.938 | 1.825 | 34.652 | 1.00 | 18.49 | A | N |
| ATOM | 1638 | CA | ASN | A | 519 | 23.671 | 1.287 | 35.802 | 1.00 | 19.30 | A | C |
| ATOM | 1640 | CB | ASN | A | 519 | 24.611 | 2.346 | 36.375 | 1.00 | 19.86 | A | C |
| ATOM | 1643 | CG | ASN | A | 519 | 25.664 | 2.759 | 35.382 | 1.00 | 21.38 | A | C |
| ATOM | 1644 | OD1 | ASN | A | 519 | 26.149 | 1.930 | 34.612 | 1.00 | 26.70 | A | O |
| ATOM | 1645 | ND2 | ASN | A | 519 | 26.012 | 4.042 | 35.372 | 1.00 | 28.25 | A | N |
| ATOM | 1648 | C | ASN | A | 519 | 22.823 | 0.691 | 36.922 | 1.00 | 19.78 | A | C |
| ATOM | 1649 | O | ASN | A | 519 | 23.344 | −0.079 | 37.710 | 1.00 | 19.38 | A | O |
| ATOM | 1650 | N | SER | A | 520 | 21.531 | 1.029 | 36.985 | 1.00 | 19.92 | A | N |
| ATOM | 1652 | CA | SER | A | 520 | 20.647 | 0.503 | 38.031 | 1.00 | 20.71 | A | C |
| ATOM | 1654 | CB | SER | A | 520 | 20.044 | 1.663 | 38.834 | 1.00 | 21.31 | A | C |
| ATOM | 1657 | OG | SER | A | 520 | 19.120 | 2.400 | 38.069 | 1.00 | 23.80 | A | O |
| ATOM | 1659 | C | SER | A | 520 | 19.536 | −0.435 | 37.536 | 1.00 | 20.17 | A | C |
| ATOM | 1660 | O | SER | A | 520 | 18.775 | −0.953 | 38.335 | 1.00 | 21.79 | A | O |
| ATOM | 1661 | N | LEU | A | 521 | 19.456 | −0.678 | 36.234 | 1.00 | 18.62 | A | N |
| ATOM | 1663 | CA | LEU | A | 521 | 18.417 | −1.545 | 35.676 | 1.00 | 17.54 | A | C |
| ATOM | 1665 | CB | LEU | A | 521 | 18.209 | −1.218 | 34.192 | 1.00 | 17.30 | A | C |
| ATOM | 1668 | CG | LEU | A | 521 | 17.417 | 0.046 | 33.909 | 1.00 | 17.34 | A | C |
| ATOM | 1670 | CD1 | LEU | A | 521 | 17.651 | 0.555 | 32.493 | 1.00 | 17.37 | A | C |
| ATOM | 1674 | CD2 | LEU | A | 521 | 15.950 | −0.230 | 34.136 | 1.00 | 19.13 | A | C |
| ATOM | 1678 | C | LEU | A | 521 | 18.794 | −3.014 | 35.804 | 1.00 | 16.93 | A | C |
| ATOM | 1679 | O | LEU | A | 521 | 19.938 | −3.378 | 35.566 | 1.00 | 17.89 | A | O |
| ATOM | 1680 | N | LYS | A | 522 | 17.841 | −3.863 | 36.164 | 1.00 | 15.75 | A | N |
| ATOM | 1682 | CA | LYS | A | 522 | 18.070 | −5.314 | 36.231 | 1.00 | 15.93 | A | C |
| ATOM | 1684 | CB | LYS | A | 522 | 17.277 | −5.944 | 37.385 | 1.00 | 16.21 | A | C |
| ATOM | 1687 | CG | LYS | A | 522 | 17.444 | −5.237 | 38.719 | 1.00 | 18.79 | A | C |
| ATOM | 1690 | CD | LYS | A | 522 | 16.528 | −5.804 | 39.802 | 1.00 | 21.17 | A | C |
| ATOM | 1693 | CE | LYS | A | 522 | 15.090 | −5.353 | 39.648 | 1.00 | 23.90 | A | C |
| ATOM | 1696 | NZ | LYS | A | 522 | 14.842 | −3.888 | 39.964 | 1.00 | 25.89 | A | N |
| ATOM | 1700 | C | LYS | A | 522 | 17.623 | −5.956 | 34.915 | 1.00 | 14.75 | A | C |
| ATOM | 1701 | O | LYS | A | 522 | 16.727 | −5.432 | 34.257 | 1.00 | 14.52 | A | O |
| ATOM | 1702 | N | VAL | A | 523 | 18.223 | −7.096 | 34.556 | 1.00 | 13.60 | A | N |
| ATOM | 1704 | CA | VAL | A | 523 | 17.821 | −7.845 | 33.349 | 1.00 | 13.60 | A | C |
| ATOM | 1706 | CB | VAL | A | 523 | 18.666 | −9.113 | 33.137 | 1.00 | 13.75 | A | C |
| ATOM | 1708 | CG1 | VAL | A | 523 | 18.182 | −9.891 | 31.939 | 1.00 | 13.84 | A | C |
| ATOM | 1712 | CG2 | VAL | A | 523 | 20.157 | −8.741 | 32.968 | 1.00 | 14.55 | A | C |
| ATOM | 1716 | C | VAL | A | 523 | 16.344 | −8.238 | 33.420 | 1.00 | 13.39 | A | C |
| ATOM | 1717 | O | VAL | A | 523 | 15.631 | −8.222 | 32.427 | 1.00 | 12.33 | A | O |
| ATOM | 1718 | N | LEU | A | 524 | 15.887 | −8.535 | 34.626 | 1.00 | 13.26 | A | N |
| ATOM | 1720 | CA | LEU | A | 524 | 14.512 | −8.886 | 34.875 | 1.00 | 14.13 | A | C |
| ATOM | 1722 | CB | LEU | A | 524 | 14.392 | −9.107 | 36.382 | 1.00 | 15.52 | A | C |
| ATOM | 1725 | CG | LEU | A | 524 | 13.090 | −9.468 | 37.032 | 1.00 | 20.72 | A | C |
| ATOM | 1727 | CD1 | LEU | A | 524 | 12.523 | −10.698 | 36.358 | 1.00 | 22.55 | A | C |
| ATOM | 1731 | CD2 | LEU | A | 524 | 13.407 | −9.706 | 38.518 | 1.00 | 22.42 | A | C |
| ATOM | 1735 | C | LEU | A | 524 | 13.556 | −7.804 | 34.362 | 1.00 | 12.61 | A | C |
| ATOM | 1736 | O | LEU | A | 524 | 12.528 | −8.109 | 33.738 | 1.00 | 11.66 | A | O |
| ATOM | 1737 | N | THR | A | 525 | 13.909 | −6.544 | 34.590 | 1.00 | 11.21 | A | N |
| ATOM | 1739 | CA | THR | A | 525 | 13.113 | −5.402 | 34.123 | 1.00 | 11.25 | A | C |
| ATOM | 1741 | CB | THR | A | 525 | 13.650 | −4.120 | 34.788 | 1.00 | 11.51 | A | C |
| ATOM | 1743 | OG1 | THR | A | 525 | 13.552 | −4.260 | 36.215 | 1.00 | 13.76 | A | O |
| ATOM | 1745 | CG2 | THR | A | 525 | 12.815 | −2.922 | 34.467 | 1.00 | 11.96 | A | C |
| ATOM | 1749 | C | THR | A | 525 | 13.124 | −5.235 | 32.600 | 1.00 | 10.93 | A | C |
| ATOM | 1750 | O | THR | A | 525 | 12.114 | −4.850 | 32.001 | 1.00 | 10.34 | A | O |
| ATOM | 1751 | N | LEU | A | 526 | 14.275 | −5.482 | 31.987 | 1.00 | 9.76 | A | N |
| ATOM | 1753 | CA | LEU | A | 526 | 14.412 | −5.405 | 30.523 | 1.00 | 9.91 | A | C |
| ATOM | 1755 | CB | LEU | A | 526 | 15.877 | −5.597 | 30.105 | 1.00 | 9.71 | A | C |
| ATOM | 1758 | CG | LEU | A | 526 | 16.878 | −4.621 | 30.717 | 1.00 | 10.00 | A | C |
| ATOM | 1760 | CD1 | LEU | A | 526 | 18.268 | −4.904 | 30.219 | 1.00 | 10.63 | A | C |
| ATOM | 1764 | CD2 | LEU | A | 526 | 16.484 | −3.208 | 30.422 | 1.00 | 10.98 | A | C |
| ATOM | 1768 | C | LEU | A | 526 | 13.520 | −6.455 | 29.836 | 1.00 | 9.82 | A | C |
| ATOM | 1769 | O | LEU | A | 526 | 12.876 | −6.176 | 28.822 | 1.00 | 8.11 | A | O |
| ATOM | 1770 | N | VAL | A | 527 | 13.475 | −7.660 | 30.411 | 1.00 | 9.84 | A | N |
| ATOM | 1772 | CA | VAL | A | 527 | 12.588 | −8.709 | 29.924 | 1.00 | 10.01 | A | C |
| ATOM | 1774 | CB | VAL | A | 527 | 12.910 | −10.075 | 30.588 | 1.00 | 10.28 | A | C |
| ATOM | 1776 | CG1 | VAL | A | 527 | 11.950 | −11.157 | 30.103 | 1.00 | 12.12 | A | C |
| ATOM | 1780 | CG2 | VAL | A | 527 | 14.326 | −10.464 | 30.275 | 1.00 | 11.61 | A | C |
| ATOM | 1784 | C | VAL | A | 527 | 11.111 | −8.316 | 30.132 | 1.00 | 9.91 | A | C |
| ATOM | 1785 | O | VAL | A | 527 | 10.286 | −8.533 | 29.247 | 1.00 | 9.20 | A | O |
| ATOM | 1786 | N | LEU | A | 528 | 10.783 | −7.715 | 31.277 | 1.00 | 9.17 | A | N |
| ATOM | 1788 | CA | LEU | A | 528 | 9.419 | −7.239 | 31.548 | 1.00 | 9.55 | A | C |
| ATOM | 1790 | CB | LEU | A | 528 | 9.308 | −6.582 | 32.940 | 1.00 | 10.47 | A | C |
| ATOM | 1793 | CG | LEU | A | 528 | 7.962 | −5.908 | 33.230 | 1.00 | 11.24 | A | C |
| ATOM | 1795 | CD1 | LEU | A | 528 | 6.855 | −6.956 | 33.158 | 1.00 | 11.66 | A | C |
| ATOM | 1799 | CD2 | LEU | A | 528 | 7.961 | −5.179 | 34.544 | 1.00 | 12.72 | A | C |
| ATOM | 1803 | C | LEU | A | 528 | 8.976 | −6.263 | 30.464 | 1.00 | 9.67 | A | C |
| ATOM | 1804 | O | LEU | A | 528 | 7.878 | −6.389 | 29.928 | 1.00 | 8.64 | A | O |
| ATOM | 1805 | N | TYR | A | 529 | 9.825 | −5.290 | 30.120 | 1.00 | 9.08 | A | N |
| ATOM | 1807 | CA | TYR | A | 529 | 9.447 | −4.301 | 29.091 | 1.00 | 8.78 | A | C |
| ATOM | 1809 | CB | TYR | A | 529 | 10.522 | −3.215 | 28.939 | 1.00 | 8.57 | A | C |
| ATOM | 1812 | CG | TYR | A | 529 | 10.744 | −2.321 | 30.143 | 1.00 | 10.12 | A | C |
| ATOM | 1813 | CD1 | TYR | A | 529 | 9.833 | −2.257 | 31.193 | 1.00 | 10.85 | A | C |
| ATOM | 1815 | CE1 | TYR | A | 529 | 10.057 | −1.438 | 32.298 | 1.00 | 12.11 | A | C |
| ATOM | 1817 | CZ | TYR | A | 529 | 11.211 | −0.677 | 32.365 | 1.00 | 13.84 | A | C |
| ATOM | 1818 | OH | TYR | A | 529 | 11.441 | 0.140 | 33.449 | 1.00 | 16.48 | A | O |
| ATOM | 1820 | CE2 | TYR | A | 529 | 12.137 | −0.737 | 31.354 | 1.00 | 11.18 | A | C |
| ATOM | 1822 | CD2 | TYR | A | 529 | 11.903 | −1.565 | 30.248 | 1.00 | 11.83 | A | C |
| ATOM | 1824 | C | TYR | A | 529 | 9.202 | −4.967 | 27.734 | 1.00 | 8.22 | A | C |
| ATOM | 1825 | O | TYR | A | 529 | 8.255 | −4.615 | 27.028 | 1.00 | 9.04 | A | O |
| ATOM | 1826 | N | SER | A | 530 | 10.036 | −5.937 | 27.390 | 1.00 | 8.24 | A | N |
| ATOM | 1828 | CA | SER | A | 530 | 9.889 | −6.722 | 26.154 | 1.00 | 7.83 | A | C |
| ATOM | 1830 | CB | SER | A | 530 | 11.052 | −7.730 | 26.015 | 1.00 | 7.54 | A | C |
| ATOM | 1833 | OG | SER | A | 530 | 12.310 | −7.077 | 25.839 | 1.00 | 10.28 | A | O |
| ATOM | 1835 | C | SER | A | 530 | 8.543 | −7.458 | 26.137 | 1.00 | 7.48 | A | C |
| ATOM | 1836 | O | SER | A | 530 | 7.820 | −7.443 | 25.141 | 1.00 | 7.17 | A | O |
| ATOM | 1837 | N | LEU | A | 531 | 8.199 | −8.074 | 27.258 | 1.00 | 6.73 | A | N |
| ATOM | 1839 | CA | LEU | A | 531 | 6.931 | −8.800 | 27.389 | 1.00 | 7.75 | A | C |
| ATOM | 1841 | CB | LEU | A | 531 | 6.912 | −9.561 | 28.728 | 1.00 | 7.60 | A | C |
| ATOM | 1844 | CG | LEU | A | 531 | 5.618 | −10.232 | 29.178 | 1.00 | 8.41 | A | C |
| ATOM | 1846 | CD1 | LEU | A | 531 | 5.201 | −11.263 | 28.153 | 1.00 | 9.01 | A | C |
| ATOM | 1850 | CD2 | LEU | A | 531 | 5.776 | −10.881 | 30.561 | 1.00 | 11.57 | A | C |
| ATOM | 1854 | C | LEU | A | 531 | 5.731 | −7.876 | 27.280 | 1.00 | 7.08 | A | C |
| ATOM | 1855 | O | LEU | A | 531 | 4.745 | −8.201 | 26.644 | 1.00 | 6.97 | A | O |
| ATOM | 1856 | N | GLN | A | 532 | 5.797 | −6.711 | 27.911 | 1.00 | 7.33 | A | N |
| ATOM | 1858 | CA | GLN | A | 532 | 4.706 | −5.735 | 27.831 | 1.00 | 7.02 | A | C |
| ATOM | 1860 | CB | GLN | A | 532 | 5.050 | −4.514 | 28.702 | 1.00 | 6.93 | A | C |
| ATOM | 1863 | CG | GLN | A | 532 | 4.930 | −4.815 | 30.195 | 1.00 | 7.50 | A | C |
| ATOM | 1866 | CD | GLN | A | 532 | 5.235 | −3.632 | 31.101 | 1.00 | 9.98 | A | C |
| ATOM | 1867 | OE1 | GLN | A | 532 | 5.756 | −2.626 | 30.657 | 1.00 | 9.61 | A | O |
| ATOM | 1868 | NE2 | GLN | A | 532 | 4.903 | −3.772 | 32.393 | 1.00 | 8.99 | A | N |
| ATOM | 1871 | C | GLN | A | 532 | 4.439 | −5.290 | 26.388 | 1.00 | 7.09 | A | C |
| ATOM | 1872 | O | GLN | A | 532 | 3.281 | −5.223 | 25.942 | 1.00 | 6.73 | A | O |
| ATOM | 1873 | N | ILE | A | 533 | 5.505 | −4.991 | 25.645 | 1.00 | 7.80 | A | N |
| ATOM | 1875 | CA | ILE | A | 533 | 5.341 | −4.572 | 24.264 | 1.00 | 8.29 | A | C |
| ATOM | 1877 | CB | ILE | A | 533 | 6.663 | −4.028 | 23.692 | 1.00 | 8.97 | A | C |
| ATOM | 1879 | CG1 | ILE | A | 533 | 7.124 | −2.779 | 24.445 | 1.00 | 8.38 | A | C |
| ATOM | 1882 | CD1 | ILE | A | 533 | 6.158 | −1.630 | 24.424 | 1.00 | 9.69 | A | C |
| ATOM | 1886 | CG2 | ILE | A | 533 | 6.527 | −3.760 | 22.221 | 1.00 | 9.41 | A | C |
| ATOM | 1890 | C | ILE | A | 533 | 4.825 | −5.736 | 23.419 | 1.00 | 8.33 | A | C |
| ATOM | 1891 | O | ILE | A | 533 | 4.010 | −5.548 | 22.512 | 1.00 | 8.38 | A | O |
| ATOM | 1892 | N | CYS | A | 534 | 5.305 | −6.938 | 23.717 | 1.00 | 7.80 | A | N |
| ATOM | 1894 | CA | CYS | A | 534 | 4.829 | −8.141 | 23.044 | 1.00 | 8.73 | A | C |
| ATOM | 1896 | CB | CYS | A | 534 | 5.588 | −9.373 | 23.521 | 1.00 | 9.35 | A | C |
| ATOM | 1899 | SG | CYS | A | 534 | 5.440 | −10.785 | 22.388 | 1.00 | 10.44 | A | S |
| ATOM | 1900 | C | CYS | A | 534 | 3.332 | −8.326 | 23.234 | 1.00 | 8.30 | A | C |
| ATOM | 1901 | O | CYS | A | 534 | 2.638 | −8.690 | 22.279 | 1.00 | 7.97 | A | O |
| ATOM | 1902 | N | LYS | A | 535 | 2.821 | −8.083 | 24.451 | 1.00 | 8.09 | A | N |
| ATOM | 1904 | CA | LYS | A | 535 | 1.381 | −8.179 | 24.685 | 1.00 | 8.26 | A | C |
| ATOM | 1906 | CB | LYS | A | 535 | 1.055 | −8.053 | 26.169 | 1.00 | 8.37 | A | C |
| ATOM | 1909 | CG | LYS | A | 535 | 1.491 | −9.239 | 26.965 | 1.00 | 10.48 | A | C |
| ATOM | 1912 | CD | LYS | A | 535 | 1.166 | −9.072 | 28.438 | 1.00 | 13.12 | A | C |
| ATOM | 1915 | CE | LYS | A | 535 | 1.380 | −10.378 | 29.159 | 1.00 | 15.75 | A | C |
| ATOM | 1918 | NZ | LYS | A | 535 | 1.022 | −10.255 | 30.588 | 1.00 | 19.99 | A | N |
| ATOM | 1922 | C | LYS | A | 535 | 0.569 | −7.160 | 23.880 | 1.00 | 8.96 | A | C |
| ATOM | 1923 | O | LYS | A | 535 | −0.525 | −7.471 | 23.383 | 1.00 | 8.89 | A | O |
| ATOM | 1924 | N | ALA | A | 536 | 1.079 | −5.946 | 23.758 | 1.00 | 8.31 | A | N |
| ATOM | 1926 | CA | ALA | A | 536 | 0.461 | −4.964 | 22.870 | 1.00 | 8.35 | A | C |
| ATOM | 1928 | CB | ALA | A | 536 | 1.208 | −3.652 | 22.932 | 1.00 | 8.92 | A | C |
| ATOM | 1932 | C | ALA | A | 536 | 0.418 | −5.463 | 21.434 | 1.00 | 8.28 | A | C |
| ATOM | 1933 | O | ALA | A | 536 | −0.595 | −5.288 | 20.745 | 1.00 | 8.64 | A | O |
| ATOM | 1934 | N | MET | A | 537 | 1.505 | −6.073 | 20.973 | 1.00 | 7.09 | A | N |
| ATOM | 1936 | CA | MET | A | 537 | 1.578 | −6.552 | 19.596 | 1.00 | 7.42 | A | C |
| ATOM | 1938 | CB | MET | A | 537 | 3.020 | −6.875 | 19.183 | 1.00 | 7.57 | A | C |
| ATOM | 1941 | CG | MET | A | 537 | 3.879 | −5.632 | 19.008 | 1.00 | 7.90 | A | C |
| ATOM | 1944 | SD | MET | A | 537 | 3.169 | −4.342 | 17.962 | 1.00 | 11.76 | A | S |
| ATOM | 1945 | CE | MET | A | 537 | 2.724 | −5.210 | 16.517 | 1.00 | 14.61 | A | C |
| ATOM | 1949 | C | MET | A | 537 | 0.679 | −7.755 | 19.365 | 1.00 | 7.65 | A | C |
| ATOM | 1950 | O | MET | A | 537 | 0.122 | −7.907 | 18.271 | 1.00 | 8.38 | A | O |
| ATOM | 1951 | N | ALA | A | 538 | 0.479 | −8.576 | 20.386 | 1.00 | 7.79 | A | N |
| ATOM | 1953 | CA | ALA | A | 538 | −0.451 | −9.717 | 20.244 | 1.00 | 8.20 | A | C |
| ATOM | 1955 | CB | ALA | A | 538 | −0.415 | −10.622 | 21.450 | 1.00 | 8.47 | A | C |
| ATOM | 1959 | C | ALA | A | 538 | −1.876 | −9.203 | 20.011 | 1.00 | 8.12 | A | C |
| ATOM | 1960 | O | ALA | A | 538 | −2.643 | −9.800 | 19.251 | 1.00 | 8.25 | A | O |
| ATOM | 1961 | N | TYR | A | 539 | −2.230 | −8.116 | 20.670 | 1.00 | 8.11 | A | N |
| ATOM | 1963 | CA | TYR | A | 539 | −3.542 | −7.483 | 20.457 | 1.00 | 9.32 | A | C |
| ATOM | 1965 | CB | TYR | A | 539 | −3.774 | −6.365 | 21.469 | 1.00 | 9.99 | A | C |
| ATOM | 1968 | CG | TYR | A | 539 | −5.068 | −5.630 | 21.207 | 1.00 | 11.93 | A | C |
| ATOM | 1969 | CD1 | TYR | A | 539 | −6.271 | −6.313 | 21.198 | 1.00 | 15.15 | A | C |
| ATOM | 1971 | CE1 | TYR | A | 539 | −7.490 | −5.653 | 20.916 | 1.00 | 19.59 | A | C |
| ATOM | 1973 | CZ | TYR | A | 539 | −7.490 | −4.300 | 20.654 | 1.00 | 21.53 | A | C |
| ATOM | 1974 | OH | TYR | A | 539 | −8.702 | −3.667 | 20.376 | 1.00 | 25.74 | A | O |
| ATOM | 1976 | CE2 | TYR | A | 539 | −6.300 | −3.595 | 20.656 | 1.00 | 19.80 | A | C |
| ATOM | 1978 | CD2 | TYR | A | 539 | −5.082 | −4.267 | 20.928 | 1.00 | 17.80 | A | C |
| ATOM | 1980 | C | TYR | A | 539 | −3.671 | −6.953 | 19.013 | 1.00 | 9.25 | A | C |
| ATOM | 1981 | O | TYR | A | 539 | −4.671 | −7.228 | 18.327 | 1.00 | 10.22 | A | O |
| ATOM | 1982 | N | LEU | A | 540 | −2.662 | −6.226 | 18.534 | 1.00 | 8.68 | A | N |
| ATOM | 1984 | CA | LEU | A | 540 | −2.681 | −5.747 | 17.162 | 1.00 | 9.52 | A | C |
| ATOM | 1986 | CB | LEU | A | 540 | −1.497 | −4.781 | 16.902 | 1.00 | 9.52 | A | C |
| ATOM | 1989 | CG | LEU | A | 540 | −1.503 | −3.503 | 17.757 | 1.00 | 9.17 | A | C |
| ATOM | 1991 | CD1 | LEU | A | 540 | −0.301 | −2.603 | 17.427 | 1.00 | 12.37 | A | C |
| ATOM | 1995 | CD2 | LEU | A | 540 | −2.795 | −2.746 | 17.652 | 1.00 | 11.42 | A | C |
| ATOM | 1999 | C | LEU | A | 540 | −2.765 | −6.896 | 16.132 | 1.00 | 10.02 | A | C |
| ATOM | 2000 | O | LEU | A | 540 | −3.496 | −6.801 | 15.139 | 1.00 | 10.77 | A | O |
| ATOM | 2001 | N | GLU | A | 541 | −2.053 | −7.983 | 16.386 | 1.00 | 9.88 | A | N |
| ATOM | 2003 | CA | GLU | A | 541 | −2.071 | −9.173 | 15.534 | 1.00 | 10.37 | A | C |
| ATOM | 2005 | CB | GLU | A | 541 | −1.081 | −10.216 | 16.087 | 1.00 | 10.24 | A | C |
| ATOM | 2008 | CG | GLU | A | 541 | −1.140 | −11.612 | 15.478 | 1.00 | 11.36 | A | C |
| ATOM | 2011 | CD | GLU | A | 541 | −0.136 | −12.569 | 16.101 | 1.00 | 10.66 | A | C |
| ATOM | 2012 | OE1 | GLU | A | 541 | −0.448 | −13.191 | 17.153 | 1.00 | 11.76 | A | O |
| ATOM | 2013 | OE2 | GLU | A | 541 | 0.968 | −12.704 | 15.543 | 1.00 | 11.66 | A | O |
| ATOM | 2014 | C | GLU | A | 541 | −3.491 | −9.739 | 15.437 | 1.00 | 10.85 | A | C |
| ATOM | 2015 | O | GLU | A | 541 | −3.907 | −10.192 | 14.378 | 1.00 | 11.81 | A | O |
| ATOM | 2016 | N | SER | A | 542 | −4.243 | −9.677 | 16.531 | 1.00 | 11.36 | A | N |
| ATOM | 2018 | CA | SER | A | 542 | −5.608 | −10.206 | 16.550 | 1.00 | 11.65 | A | C |
| ATOM | 2020 | CB | SER | A | 542 | −6.160 | −10.250 | 17.976 | 1.00 | 12.06 | A | C |
| ATOM | 2023 | OG | SER | A | 542 | −6.549 | −8.983 | 18.468 | 1.00 | 11.97 | A | O |
| ATOM | 2025 | C | SER | A | 542 | −6.576 | −9.458 | 15.641 | 1.00 | 12.56 | A | C |
| ATOM | 2026 | O | SER | A | 542 | −7.631 | −10.010 | 15.277 | 1.00 | 11.49 | A | O |
| ATOM | 2027 | N | ILE | A | 543 | −6.260 | −8.210 | 15.323 | 1.00 | 12.89 | A | N |
| ATOM | 2029 | CA | ILE | A | 543 | −7.050 | −7.424 | 14.358 | 1.00 | 13.67 | A | C |
| ATOM | 2031 | CB | ILE | A | 543 | −7.562 | −6.115 | 14.994 | 1.00 | 14.02 | A | C |
| ATOM | 2033 | CG1 | ILE | A | 543 | −6.433 | −5.283 | 15.598 | 1.00 | 14.22 | A | C |
| ATOM | 2036 | CD1 | ILE | A | 543 | −6.826 | −3.900 | 15.926 | 1.00 | 15.54 | A | C |
| ATOM | 2040 | CG2 | ILE | A | 543 | −8.591 | −6.404 | 16.074 | 1.00 | 13.84 | A | C |
| ATOM | 2044 | C | ILE | A | 543 | −6.317 | −7.159 | 13.042 | 1.00 | 14.47 | A | C |
| ATOM | 2045 | O | ILE | A | 543 | −6.732 | −6.316 | 12.252 | 1.00 | 14.15 | A | O |
| ATOM | 2046 | N | ASN | A | 544 | −5.235 | −7.885 | 12.809 | 1.00 | 15.25 | A | N |
| ATOM | 2048 | CA | ASN | A | 544 | −4.457 | −7.780 | 11.575 | 1.00 | 17.01 | A | C |
| ATOM | 2050 | CB | ASN | A | 544 | −5.260 | −8.311 | 10.368 | 1.00 | 18.38 | A | C |
| ATOM | 2053 | CG | ASN | A | 544 | −5.668 | −9.758 | 10.523 | 1.00 | 22.26 | A | C |
| ATOM | 2054 | OD1 | ASN | A | 544 | −4.821 | −10.644 | 10.660 | 1.00 | 28.79 | A | O |
| ATOM | 2055 | ND2 | ASN | A | 544 | −6.970 | −10.012 | 10.491 | 1.00 | 27.53 | A | N |
| ATOM | 2058 | C | ASN | A | 544 | −3.995 | −6.353 | 11.302 | 1.00 | 17.05 | A | C |
| ATOM | 2059 | O | ASN | A | 544 | −4.019 | −5.878 | 10.162 | 1.00 | 18.03 | A | O |
| ATOM | 2060 | N | CYS | A | 545 | −3.591 | −5.672 | 12.365 | 1.00 | 16.00 | A | N |
| ATOM | 2062 | CA | CYS | A | 545 | −3.103 | −4.305 | 12.291 | 1.00 | 16.72 | A | C |
| ATOM | 2064 | CB | CYS | A | 545 | −3.628 | −3.544 | 13.494 | 1.00 | 16.76 | A | C |
| ATOM | 2067 | SG | CYS | A | 545 | −3.019 | −1.887 | 13.701 | 1.00 | 24.49 | A | S |
| ATOM | 2068 | C | CYS | A | 545 | −1.586 | −4.358 | 12.295 | 1.00 | 15.33 | A | C |
| ATOM | 2069 | O | CYS | A | 545 | −0.994 | −4.800 | 13.278 | 1.00 | 15.78 | A | O |
| ATOM | 2070 | N | VAL | A | 546 | −0.969 | −3.933 | 11.194 | 1.00 | 14.28 | A | N |
| ATOM | 2072 | CA | VAL | A | 546 | 0.498 | −3.913 | 11.064 | 1.00 | 13.55 | A | C |
| ATOM | 2074 | CB | VAL | A | 546 | 0.939 | −4.206 | 9.610 | 1.00 | 14.14 | A | C |
| ATOM | 2076 | CG1 | VAL | A | 546 | 2.466 | −4.261 | 9.507 | 1.00 | 15.81 | A | C |
| ATOM | 2080 | CG2 | VAL | A | 546 | 0.296 | −5.520 | 9.111 | 1.00 | 15.91 | A | C |
| ATOM | 2084 | C | VAL | A | 546 | 0.998 | −2.542 | 11.524 | 1.00 | 13.04 | A | C |
| ATOM | 2085 | O | VAL | A | 546 | 0.606 | −1.505 | 10.980 | 1.00 | 12.91 | A | O |
| ATOM | 2086 | N | HIS | A | 547 | 1.871 | −2.540 | 12.533 | 1.00 | 11.11 | A | N |
| ATOM | 2088 | CA | HIS | A | 547 | 2.287 | −1.318 | 13.227 | 1.00 | 10.91 | A | C |
| ATOM | 2090 | CB | HIS | A | 547 | 2.643 | −1.668 | 14.683 | 1.00 | 10.19 | A | C |
| ATOM | 2093 | CG | HIS | A | 547 | 2.902 | −0.475 | 15.544 | 1.00 | 10.34 | A | C |
| ATOM | 2094 | ND1 | HIS | A | 547 | 4.081 | 0.228 | 15.491 | 1.00 | 11.48 | A | N |
| ATOM | 2096 | CE1 | HIS | A | 547 | 4.038 | 1.226 | 16.361 | 1.00 | 10.70 | A | C |
| ATOM | 2098 | NE2 | HIS | A | 547 | 2.864 | 1.199 | 16.967 | 1.00 | 9.34 | A | N |
| ATOM | 2100 | CD2 | HIS | A | 547 | 2.140 | 0.138 | 16.481 | 1.00 | 10.35 | A | C |
| ATOM | 2102 | C | HIS | A | 547 | 3.431 | −0.584 | 12.527 | 1.00 | 10.89 | A | C |
| ATOM | 2103 | O | HIS | A | 547 | 3.342 | 0.628 | 12.322 | 1.00 | 11.28 | A | O |
| ATOM | 2104 | N | ARG | A | 548 | 4.479 | −1.321 | 12.157 | 1.00 | 10.62 | A | N |
| ATOM | 2106 | CA | ARG | A | 548 | 5.652 | −0.819 | 11.406 | 1.00 | 10.76 | A | C |
| ATOM | 2108 | CB | ARG | A | 548 | 5.248 | −0.092 | 10.114 | 1.00 | 11.42 | A | C |
| ATOM | 2111 | CG | ARG | A | 548 | 4.346 | −0.845 | 9.128 | 1.00 | 12.89 | A | C |
| ATOM | 2114 | CD | ARG | A | 548 | 4.246 | −0.093 | 7.781 | 1.00 | 15.96 | A | C |
| ATOM | 2117 | NE | ARG | A | 548 | 3.297 | −0.686 | 6.841 | 1.00 | 16.19 | A | N |
| ATOM | 2119 | CZ | ARG | A | 548 | 3.236 | −0.367 | 5.543 | 1.00 | 18.71 | A | C |
| ATOM | 2120 | NH1 | ARG | A | 548 | 4.045 | 0.549 | 5.039 | 1.00 | 17.01 | A | N |
| ATOM | 2123 | NH2 | ARG | A | 548 | 2.350 | −0.948 | 4.752 | 1.00 | 19.52 | A | N |
| ATOM | 2126 | C | ARG | A | 548 | 6.616 | 0.100 | 12.165 | 1.00 | 10.94 | A | C |
| ATOM | 2127 | O | ARG | A | 548 | 7.610 | 0.540 | 11.585 | 1.00 | 11.17 | A | O |
| ATOM | 2128 | N | ASP | A | 549 | 6.345 | 0.423 | 13.421 | 1.00 | 10.64 | A | N |
| ATOM | 2130 | CA | ASP | A | 549 | 7.255 | 1.294 | 14.181 | 1.00 | 11.41 | A | C |
| ATOM | 2132 | CB | ASP | A | 549 | 6.824 | 2.767 | 14.120 | 1.00 | 11.59 | A | C |
| ATOM | 2135 | CG | ASP | A | 549 | 7.947 | 3.735 | 14.497 | 1.00 | 16.46 | A | C |
| ATOM | 2136 | OD1 | ASP | A | 549 | 9.129 | 3.319 | 14.543 | 1.00 | 19.23 | A | O |
| ATOM | 2137 | OD2 | ASP | A | 549 | 7.726 | 4.944 | 14.778 | 1.00 | 18.02 | A | O |
| ATOM | 2138 | C | ASP | A | 549 | 7.456 | 0.836 | 15.616 | 1.00 | 10.41 | A | C |
| ATOM | 2139 | O | ASP | A | 549 | 7.269 | 1.597 | 16.576 | 1.00 | 10.29 | A | O |
| ATOM | 2140 | N | ILE | A | 550 | 7.926 | −0.400 | 15.743 | 1.00 | 10.29 | A | N |
| ATOM | 2142 | CA | ILE | A | 550 | 8.229 | −0.986 | 17.046 | 1.00 | 10.14 | A | C |
| ATOM | 2144 | CB | ILE | A | 550 | 7.858 | −2.490 | 17.037 | 1.00 | 10.18 | A | C |
| ATOM | 2146 | CG1 | ILE | A | 550 | 6.440 | −2.685 | 16.476 | 1.00 | 9.59 | A | C |
| ATOM | 2149 | CD1 | ILE | A | 550 | 6.199 | −4.070 | 15.917 | 1.00 | 12.93 | A | C |
| ATOM | 2153 | CG2 | ILE | A | 550 | 7.936 | −3.059 | 18.435 | 1.00 | 10.80 | A | C |
| ATOM | 2157 | C | ILE | A | 550 | 9.709 | −0.791 | 17.310 | 1.00 | 10.07 | A | C |
| ATOM | 2158 | O | ILE | A | 550 | 10.517 | −1.517 | 16.793 | 1.00 | 10.23 | A | O |
| ATOM | 2159 | N | ALA | A | 551 | 10.032 | 0.227 | 18.094 | 1.00 | 10.23 | A | N |
| ATOM | 2161 | CA | ALA | A | 551 | 11.403 | 0.671 | 18.312 | 1.00 | 9.79 | A | C |
| ATOM | 2163 | CB | ALA | A | 551 | 11.883 | 1.526 | 17.130 | 1.00 | 9.99 | A | C |
| ATOM | 2167 | C | ALA | A | 551 | 11.404 | 1.495 | 19.593 | 1.00 | 9.51 | A | C |
| ATOM | 2168 | O | ALA | A | 551 | 10.399 | 2.111 | 19.911 | 1.00 | 8.53 | A | O |
| ATOM | 2169 | N | VAL | A | 552 | 12.536 | 1.529 | 20.304 | 1.00 | 8.79 | A | N |
| ATOM | 2171 | CA | VAL | A | 552 | 12.595 | 2.169 | 21.630 | 1.00 | 9.71 | A | C |
| ATOM | 2173 | CB | VAL | A | 552 | 13.917 | 1.852 | 22.434 | 1.00 | 9.32 | A | C |
| ATOM | 2175 | CG1 | VAL | A | 552 | 13.981 | 0.377 | 22.814 | 1.00 | 11.97 | A | C |
| ATOM | 2179 | CG2 | VAL | A | 552 | 15.175 | 2.296 | 21.700 | 1.00 | 10.51 | A | C |
| ATOM | 2183 | C | VAL | A | 552 | 12.373 | 3.671 | 21.605 | 1.00 | 9.86 | A | C |
| ATOM | 2184 | O | VAL | A | 552 | 11.910 | 4.231 | 22.605 | 1.00 | 9.34 | A | O |
| ATOM | 2185 | N | ARG | A | 553 | 12.672 | 4.305 | 20.462 | 1.00 | 9.93 | A | N |
| ATOM | 2187 | CA | ARG | A | 553 | 12.381 | 5.715 | 20.233 | 1.00 | 11.22 | A | C |
| ATOM | 2189 | CB | ARG | A | 553 | 12.914 | 6.152 | 18.849 | 1.00 | 12.32 | A | C |
| ATOM | 2192 | CG | ARG | A | 553 | 12.746 | 7.598 | 18.520 | 1.00 | 18.30 | A | C |
| ATOM | 2195 | CD | ARG | A | 553 | 13.541 | 8.051 | 17.284 | 1.00 | 25.13 | A | C |
| ATOM | 2198 | NE | ARG | A | 553 | 13.202 | 9.422 | 16.907 | 1.00 | 30.79 | A | N |
| ATOM | 2200 | CZ | ARG | A | 553 | 13.974 | 10.246 | 16.190 | 1.00 | 34.09 | A | C |
| ATOM | 2201 | NH1 | ARG | A | 553 | 15.161 | 9.865 | 15.721 | 1.00 | 35.69 | A | N |
| ATOM | 2204 | NH2 | ARG | A | 553 | 13.536 | 11.475 | 15.928 | 1.00 | 35.84 | A | N |
| ATOM | 2207 | C | ARG | A | 553 | 10.873 | 5.962 | 20.295 | 1.00 | 10.85 | A | C |
| ATOM | 2208 | O | ARG | A | 553 | 10.427 | 7.061 | 20.646 | 1.00 | 9.83 | A | O |
| ATOM | 2209 | N | ASN | A | 554 | 10.101 | 4.948 | 19.911 | 1.00 | 8.85 | A | N |
| ATOM | 2211 | CA | ASN | A | 554 | 8.650 | 5.050 | 19.820 | 1.00 | 9.54 | A | C |
| ATOM | 2213 | CB | ASN | A | 554 | 8.173 | 4.595 | 18.434 | 1.00 | 8.92 | A | C |
| ATOM | 2216 | CG | ASN | A | 554 | 6.729 | 4.998 | 18.148 | 1.00 | 10.60 | A | C |
| ATOM | 2217 | OD1 | ASN | A | 554 | 6.321 | 6.131 | 18.457 | 1.00 | 11.60 | A | O |
| ATOM | 2218 | ND2 | ASN | A | 554 | 5.959 | 4.101 | 17.560 | 1.00 | 11.34 | A | N |
| ATOM | 2221 | C | ASN | A | 554 | 7.907 | 4.279 | 20.910 | 1.00 | 9.30 | A | C |
| ATOM | 2222 | O | ASN | A | 554 | 6.788 | 3.841 | 20.707 | 1.00 | 10.60 | A | O |
| ATOM | 2223 | N | ILE | A | 555 | 8.544 | 4.108 | 22.062 | 1.00 | 10.21 | A | N |
| ATOM | 2225 | CA | ILE | A | 555 | 7.935 | 3.514 | 23.235 | 1.00 | 9.86 | A | C |
| ATOM | 2227 | CB | ILE | A | 555 | 8.595 | 2.177 | 23.597 | 1.00 | 9.61 | A | C |
| ATOM | 2229 | CG1 | ILE | A | 555 | 8.358 | 1.140 | 22.488 | 1.00 | 10.41 | A | C |
| ATOM | 2232 | CD1 | ILE | A | 555 | 9.318 | 0.017 | 22.555 | 1.00 | 11.60 | A | C |
| ATOM | 2236 | CG2 | ILE | A | 555 | 8.103 | 1.672 | 24.948 | 1.00 | 9.20 | A | C |
| ATOM | 2240 | C | ILE | A | 555 | 8.114 | 4.536 | 24.350 | 1.00 | 10.75 | A | C |
| ATOM | 2241 | O | ILE | A | 555 | 9.208 | 5.071 | 24.541 | 1.00 | 10.55 | A | O |
| ATOM | 2242 | N | LEU | A | 556 | 7.044 | 4.807 | 25.088 | 1.00 | 10.73 | A | N |
| ATOM | 2244 | CA | LEU | A | 556 | 7.084 | 5.751 | 26.209 | 1.00 | 11.14 | A | C |
| ATOM | 2246 | CB | LEU | A | 556 | 5.932 | 6.765 | 26.100 | 1.00 | 11.83 | A | C |
| ATOM | 2249 | CG | LEU | A | 556 | 6.005 | 7.814 | 24.986 | 1.00 | 14.83 | A | C |
| ATOM | 2251 | CD1 | LEU | A | 556 | 7.308 | 8.567 | 24.986 | 1.00 | 18.18 | A | C |
| ATOM | 2255 | CD2 | LEU | A | 556 | 5.770 | 7.191 | 23.644 | 1.00 | 19.54 | A | C |
| ATOM | 2259 | C | LEU | A | 556 | 7.001 | 5.052 | 27.535 | 1.00 | 10.30 | A | C |
| ATOM | 2260 | O | LEU | A | 556 | 6.353 | 4.017 | 27.670 | 1.00 | 9.91 | A | O |
| ATOM | 2261 | N | VAL | A | 557 | 7.636 | 5.660 | 28.529 | 1.00 | 9.82 | A | N |
| ATOM | 2263 | CA | VAL | A | 557 | 7.790 | 5.088 | 29.856 | 1.00 | 10.20 | A | C |
| ATOM | 2265 | CB | VAL | A | 557 | 9.244 | 5.235 | 30.379 | 1.00 | 9.42 | A | C |
| ATOM | 2267 | CG1 | VAL | A | 557 | 9.382 | 4.662 | 31.776 | 1.00 | 9.30 | A | C |
| ATOM | 2271 | CG2 | VAL | A | 557 | 10.274 | 4.537 | 29.435 | 1.00 | 10.82 | A | C |
| ATOM | 2275 | C | VAL | A | 557 | 6.809 | 5.808 | 30.788 | 1.00 | 10.92 | A | C |
| ATOM | 2276 | O | VAL | A | 557 | 6.990 | 6.979 | 31.126 | 1.00 | 10.29 | A | O |
| ATOM | 2277 | N | ALA | A | 558 | 5.744 | 5.103 | 31.149 | 1.00 | 11.83 | A | N |
| ATOM | 2279 | CA | ALA | A | 558 | 4.753 | 5.595 | 32.116 | 1.00 | 12.73 | A | C |
| ATOM | 2281 | CB | ALA | A | 558 | 3.474 | 4.716 | 32.040 | 1.00 | 12.91 | A | C |
| ATOM | 2285 | C | ALA | A | 558 | 5.291 | 5.615 | 33.542 | 1.00 | 13.27 | A | C |
| ATOM | 2286 | O | ALA | A | 558 | 5.043 | 6.550 | 34.330 | 1.00 | 14.71 | A | O |
| ATOM | 2287 | N | SER | A | 559 | 6.010 | 4.559 | 33.884 | 1.00 | 13.11 | A | N |
| ATOM | 2289 | CA | SER | A | 559 | 6.639 | 4.396 | 35.186 | 1.00 | 13.68 | A | C |
| ATOM | 2291 | CB | SER | A | 559 | 5.618 | 3.934 | 36.220 | 1.00 | 13.21 | A | C |
| ATOM | 2294 | OG | SER | A | 559 | 5.230 | 2.589 | 36.006 | 1.00 | 13.73 | A | O |
| ATOM | 2296 | C | SER | A | 559 | 7.719 | 3.352 | 35.030 | 1.00 | 13.54 | A | C |
| ATOM | 2297 | O | SER | A | 559 | 7.737 | 2.663 | 34.025 | 1.00 | 12.55 | A | O |
| ATOM | 2298 | N | PRO | A | 560 | 8.593 | 3.177 | 36.018 | 1.00 | 14.61 | A | N |
| ATOM | 2299 | CA | PRO | A | 560 | 9.559 | 2.069 | 35.950 | 1.00 | 15.41 | A | C |
| ATOM | 2301 | CB | PRO | A | 560 | 10.361 | 2.217 | 37.251 | 1.00 | 15.46 | A | C |
| ATOM | 2304 | CG | PRO | A | 560 | 10.251 | 3.662 | 37.572 | 1.00 | 15.49 | A | C |
| ATOM | 2307 | CD | PRO | A | 560 | 8.794 | 3.996 | 37.233 | 1.00 | 15.51 | A | C |
| ATOM | 2310 | C | PRO | A | 560 | 8.919 | 0.678 | 35.800 | 1.00 | 15.83 | A | C |
| ATOM | 2311 | O | PRO | A | 560 | 9.589 | −0.279 | 35.390 | 1.00 | 16.34 | A | O |
| ATOM | 2312 | N | GLU | A | 561 | 7.626 | 0.579 | 36.070 | 1.00 | 16.53 | A | N |
| ATOM | 2314 | CA | GLU | A | 561 | 6.918 | −0.689 | 35.993 | 1.00 | 16.63 | A | C |
| ATOM | 2316 | CB | GLU | A | 561 | 6.035 | −0.834 | 37.234 | 1.00 | 18.01 | A | C |
| ATOM | 2319 | CG | GLU | A | 561 | 6.833 | −0.946 | 38.529 | 1.00 | 22.37 | A | C |
| ATOM | 2322 | CD | GLU | A | 561 | 6.517 | 0.168 | 39.506 | 1.00 | 28.24 | A | C |
| ATOM | 2323 | OE1 | GLU | A | 561 | 6.453 | 1.357 | 39.072 | 1.00 | 32.07 | A | O |
| ATOM | 2324 | OE2 | GLU | A | 561 | 6.315 | −0.152 | 40.707 | 1.00 | 33.30 | A | O |
| ATOM | 2325 | C | GLU | A | 561 | 6.059 | −0.871 | 34.733 | 1.00 | 14.93 | A | C |
| ATOM | 2326 | O | GLU | A | 561 | 5.445 | −1.916 | 34.574 | 1.00 | 14.59 | A | O |
| ATOM | 2327 | N | CYS | A | 562 | 6.025 | 0.111 | 33.835 | 1.00 | 13.75 | A | N |
| ATOM | 2329 | CA | CYS | A | 562 | 5.074 | 0.089 | 32.711 | 1.00 | 14.08 | A | C |
| ATOM | 2331 | CB | CYS | A | 562 | 3.692 | 0.541 | 33.159 | 1.00 | 14.16 | A | C |
| ATOM | 2334 | SG | CYS | A | 562 | 2.464 | 0.485 | 31.846 | 1.00 | 17.94 | A | S |
| ATOM | 2335 | C | CYS | A | 562 | 5.534 | 0.942 | 31.520 | 1.00 | 12.60 | A | C |
| ATOM | 2336 | O | CYS | A | 562 | 5.747 | 2.155 | 31.640 | 1.00 | 12.45 | A | O |
| ATOM | 2337 | N | VAL | A | 563 | 5.709 | 0.282 | 30.377 | 1.00 | 11.25 | A | N |
| ATOM | 2339 | CA | VAL | A | 563 | 5.979 | 0.962 | 29.125 | 1.00 | 10.38 | A | C |
| ATOM | 2341 | CB | VAL | A | 563 | 7.259 | 0.421 | 28.421 | 1.00 | 10.60 | A | C |
| ATOM | 2343 | CG1 | VAL | A | 563 | 8.477 | 0.548 | 29.329 | 1.00 | 10.27 | A | C |
| ATOM | 2347 | CG2 | VAL | A | 563 | 7.102 | −1.020 | 27.949 | 1.00 | 10.55 | A | C |
| ATOM | 2351 | C | VAL | A | 563 | 4.748 | 0.912 | 28.184 | 1.00 | 10.67 | A | C |
| ATOM | 2352 | O | VAL | A | 563 | 3.867 | 0.058 | 28.336 | 1.00 | 10.13 | A | O |
| ATOM | 2353 | N | LYS | A | 564 | 4.723 | 1.806 | 27.202 | 1.00 | 9.96 | A | N |
| ATOM | 2355 | CA | LYS | A | 564 | 3.586 | 1.997 | 26.285 | 1.00 | 10.71 | A | C |
| ATOM | 2357 | CB | LYS | A | 564 | 2.835 | 3.290 | 26.642 | 1.00 | 10.90 | A | C |
| ATOM | 2360 | CG | LYS | A | 564 | 2.453 | 3.420 | 28.104 | 1.00 | 13.45 | A | C |
| ATOM | 2363 | CD | LYS | A | 564 | 1.102 | 2.850 | 28.403 | 1.00 | 14.37 | A | C |
| ATOM | 2366 | CE | LYS | A | 564 | 0.676 | 3.158 | 29.838 | 1.00 | 16.32 | A | C |
| ATOM | 2369 | NZ | LYS | A | 564 | −0.589 | 2.458 | 30.221 | 1.00 | 17.26 | A | N |
| ATOM | 2373 | C | LYS | A | 564 | 4.050 | 2.148 | 24.828 | 1.00 | 9.71 | A | C |
| ATOM | 2374 | O | LYS | A | 564 | 4.814 | 3.069 | 24.502 | 1.00 | 9.88 | A | O |
| ATOM | 2375 | N | LEU | A | 565 | 3.615 | 1.238 | 23.964 | 1.00 | 8.95 | A | N |
| ATOM | 2377 | CA | LEU | A | 565 | 3.909 | 1.334 | 22.533 | 1.00 | 9.28 | A | C |
| ATOM | 2379 | CB | LEU | A | 565 | 3.418 | 0.094 | 21.802 | 1.00 | 9.45 | A | C |
| ATOM | 2382 | CG | LEU | A | 565 | 3.702 | 0.004 | 20.289 | 1.00 | 9.53 | A | C |
| ATOM | 2384 | CD1 | LEU | A | 565 | 5.202 | 0.062 | 20.003 | 1.00 | 10.72 | A | C |
| ATOM | 2388 | CD2 | LEU | A | 565 | 3.070 | −1.258 | 19.781 | 1.00 | 12.45 | A | C |
| ATOM | 2392 | C | LEU | A | 565 | 3.245 | 2.565 | 21.928 | 1.00 | 9.46 | A | C |
| ATOM | 2393 | O | LEU | A | 565 | 2.054 | 2.825 | 22.167 | 1.00 | 9.53 | A | O |
| ATOM | 2394 | N | GLY | A | 566 | 4.007 | 3.302 | 21.131 | 1.00 | 10.94 | A | N |
| ATOM | 2396 | CA | GLY | A | 566 | 3.540 | 4.539 | 20.511 | 1.00 | 12.38 | A | C |
| ATOM | 2399 | C | GLY | A | 566 | 2.748 | 4.337 | 19.222 | 1.00 | 14.40 | A | C |
| ATOM | 2400 | O | GLY | A | 566 | 2.312 | 3.235 | 18.882 | 1.00 | 12.68 | A | O |
| ATOM | 2401 | N | ASP | A | 567 | 2.524 | 5.424 | 18.494 | 1.00 | 17.06 | A | N |
| ATOM | 2403 | CA | ASP | A | 567 | 1.644 | 5.322 | 17.327 | 1.00 | 19.73 | A | C |
| ATOM | 2405 | CB | ASP | A | 567 | 0.986 | 6.649 | 16.942 | 1.00 | 21.13 | A | C |
| ATOM | 2408 | CG | ASP | A | 567 | 1.917 | 7.779 | 16.889 | 1.00 | 24.80 | A | C |
| ATOM | 2409 | OD1 | ASP | A | 567 | 2.981 | 7.672 | 16.231 | 1.00 | 32.54 | A | O |
| ATOM | 2410 | OD2 | ASP | A | 567 | 1.623 | 8.856 | 17.439 | 1.00 | 29.95 | A | O |
| ATOM | 2411 | C | ASP | A | 567 | 2.257 | 4.622 | 16.108 | 1.00 | 20.41 | A | C |
| ATOM | 2412 | O | ASP | A | 567 | 3.467 | 4.413 | 16.011 | 1.00 | 17.31 | A | O |
| ATOM | 2413 | N | PHE | A | 568 | 1.368 | 4.262 | 15.196 | 1.00 | 22.78 | A | N |
| ATOM | 2415 | CA | PHE | A | 568 | 1.698 | 3.525 | 13.979 | 1.00 | 25.32 | A | C |
| ATOM | 2417 | CB | PHE | A | 568 | 0.428 | 3.274 | 13.152 | 1.00 | 26.08 | A | C |
| ATOM | 2420 | CG | PHE | A | 568 | −0.619 | 2.555 | 13.894 | 1.00 | 27.54 | A | C |
| ATOM | 2421 | CD1 | PHE | A | 568 | −1.780 | 3.199 | 14.285 | 1.00 | 29.54 | A | C |
| ATOM | 2423 | CE1 | PHE | A | 568 | −2.745 | 2.522 | 14.983 | 1.00 | 29.57 | A | C |
| ATOM | 2425 | CZ | PHE | A | 568 | −2.560 | 1.197 | 15.312 | 1.00 | 30.42 | A | C |
| ATOM | 2427 | CE2 | PHE | A | 568 | −1.412 | 0.548 | 14.935 | 1.00 | 30.12 | A | C |
| ATOM | 2429 | CD2 | PHE | A | 568 | −0.443 | 1.228 | 14.228 | 1.00 | 29.43 | A | C |
| ATOM | 2431 | C | PHE | A | 568 | 2.666 | 4.282 | 13.116 | 1.00 | 27.26 | A | C |
| ATOM | 2432 | O | PHE | A | 568 | 2.610 | 5.502 | 13.050 | 1.00 | 27.24 | A | O |
| ATOM | 2433 | N | GLY | A | 569 | 3.536 | 3.545 | 12.433 | 1.00 | 29.69 | A | N |
| ATOM | 2435 | CA | GLY | A | 569 | 4.511 | 4.124 | 11.531 | 1.00 | 32.08 | A | C |
| ATOM | 2438 | C | GLY | A | 569 | 3.937 | 5.058 | 10.482 | 1.00 | 34.61 | A | C |
| ATOM | 2439 | O | GLY | A | 569 | 4.602 | 6.029 | 10.110 | 1.00 | 34.79 | A | O |
| ATOM | 2440 | N | LEU | A | 570 | 2.724 | 4.754 | 10.010 | 1.00 | 37.79 | A | N |
| ATOM | 2442 | CA | LEU | A | 570 | 1.999 | 5.549 | 8.993 | 1.00 | 40.50 | A | C |
| ATOM | 2444 | CB | LEU | A | 570 | 0.514 | 5.665 | 9.387 | 1.00 | 40.83 | A | C |
| ATOM | 2447 | CG | LEU | A | 570 | −0.463 | 6.442 | 8.489 | 1.00 | 41.93 | A | C |
| ATOM | 2449 | CD1 | LEU | A | 570 | −0.487 | 5.925 | 7.051 | 1.00 | 42.79 | A | C |
| ATOM | 2453 | CD2 | LEU | A | 570 | −1.870 | 6.383 | 9.095 | 1.00 | 42.81 | A | C |
| ATOM | 2457 | C | LEU | A | 570 | 2.572 | 6.949 | 8.737 | 1.00 | 42.14 | A | C |
| ATOM | 2458 | O | LEU | A | 570 | 2.682 | 7.769 | 9.670 | 1.00 | 43.08 | A | O |
| ATOM | 2459 | N | SER | A | 571 | 2.929 | 7.207 | 7.475 | 1.00 | 43.89 | A | N |
| ATOM | 2461 | CA | SER | A | 571 | 3.588 | 8.455 | 7.062 | 1.00 | 45.06 | A | C |
| ATOM | 2463 | CB | SER | A | 571 | 4.686 | 8.163 | 6.017 | 1.00 | 45.02 | A | C |
| ATOM | 2466 | OG | SER | A | 571 | 5.526 | 7.091 | 6.423 | 1.00 | 45.78 | A | O |
| ATOM | 2468 | C | SER | A | 571 | 2.551 | 9.456 | 6.521 | 1.00 | 46.00 | A | C |
| ATOM | 2469 | O | SER | A | 571 | 1.345 | 9.295 | 6.762 | 1.00 | 46.79 | A | O |
| ATOM | 2470 | N | ARG | A | 572 | 3.012 | 10.481 | 5.800 | 1.00 | 46.90 | A | N |
| ATOM | 2472 | CA | ARG | A | 572 | 2.146 | 11.585 | 5.348 | 1.00 | 47.52 | A | C |
| ATOM | 2474 | CB | ARG | A | 572 | 2.414 | 12.856 | 6.181 | 1.00 | 47.67 | A | C |
| ATOM | 2477 | CG | ARG | A | 572 | 3.899 | 13.255 | 6.343 | 1.00 | 48.36 | A | C |
| ATOM | 2480 | CD | ARG | A | 572 | 4.392 | 14.299 | 5.342 | 1.00 | 48.94 | A | C |
| ATOM | 2483 | NE | ARG | A | 572 | 5.846 | 14.476 | 5.371 | 1.00 | 49.60 | A | N |
| ATOM | 2485 | CZ | ARG | A | 572 | 6.548 | 15.158 | 4.461 | 1.00 | 49.93 | A | C |
| ATOM | 2486 | NH1 | ARG | A | 572 | 5.942 | 15.748 | 3.432 | 1.00 | 49.93 | A | N |
| ATOM | 2489 | NH2 | ARG | A | 572 | 7.869 | 15.255 | 4.580 | 1.00 | 50.04 | A | N |
| ATOM | 2492 | C | ARG | A | 572 | 2.299 | 11.870 | 3.842 | 1.00 | 47.78 | A | C |
| ATOM | 2493 | O | ARG | A | 572 | 2.330 | 13.032 | 3.413 | 1.00 | 47.93 | A | O |
| ATOM | 2494 | N | TYR | A | 573 | 2.348 | 10.800 | 3.047 | 1.00 | 47.99 | A | N |
| ATOM | 2496 | CA | TYR | A | 573 | 2.606 | 10.890 | 1.604 | 1.00 | 48.13 | A | C |
| ATOM | 2498 | CB | TYR | A | 573 | 3.839 | 10.038 | 1.264 | 1.00 | 48.32 | A | C |
| ATOM | 2501 | CG | TYR | A | 573 | 5.090 | 10.843 | 0.971 | 1.00 | 49.01 | A | C |
| ATOM | 2502 | CD1 | TYR | A | 573 | 5.840 | 11.408 | 2.003 | 1.00 | 49.66 | A | C |
| ATOM | 2504 | CE1 | TYR | A | 573 | 6.988 | 12.154 | 1.736 | 1.00 | 50.12 | A | C |
| ATOM | 2506 | CZ | TYR | A | 573 | 7.396 | 12.335 | 0.421 | 1.00 | 50.65 | A | C |
| ATOM | 2507 | OH | TYR | A | 573 | 8.530 | 13.070 | 0.141 | 1.00 | 51.55 | A | O |
| ATOM | 2509 | CE2 | TYR | A | 573 | 6.668 | 11.784 | −0.618 | 1.00 | 50.16 | A | C |
| ATOM | 2511 | CD2 | TYR | A | 573 | 5.522 | 11.041 | −0.340 | 1.00 | 49.75 | A | C |
| ATOM | 2513 | C | TYR | A | 573 | 1.396 | 10.437 | 0.749 | 1.00 | 47.99 | A | C |
| ATOM | 2514 | O | TYR | A | 573 | 0.637 | 9.549 | 1.157 | 1.00 | 48.18 | A | O |
| ATOM | 2515 | N | ILE | A | 574 | 1.218 | 11.060 | −0.423 | 1.00 | 47.68 | A | N |
| ATOM | 2517 | CA | ILE | A | 574 | 0.221 | 10.611 | −1.414 | 1.00 | 47.28 | A | C |
| ATOM | 2519 | CB | ILE | A | 574 | −0.335 | 11.823 | −2.263 | 1.00 | 47.36 | A | C |
| ATOM | 2521 | CG1 | ILE | A | 574 | −1.852 | 11.681 | −2.521 | 1.00 | 47.24 | A | C |
| ATOM | 2524 | CD1 | ILE | A | 574 | −2.265 | 10.667 | −3.589 | 1.00 | 47.19 | A | C |
| ATOM | 2528 | CG2 | ILE | A | 574 | 0.468 | 12.038 | −3.572 | 1.00 | 47.44 | A | C |
| ATOM | 2532 | C | ILE | A | 574 | 0.819 | 9.524 | −2.312 | 1.00 | 46.58 | A | C |
| ATOM | 2533 | O | ILE | A | 574 | 0.097 | 8.640 | −2.793 | 1.00 | 47.04 | A | O |
| ATOM | 2534 | N | GLU | A | 575 | 2.133 | 9.598 | −2.539 | 1.00 | 45.52 | A | N |
| ATOM | 2536 | CA | GLU | A | 575 | 2.848 | 8.569 | −3.297 | 1.00 | 44.42 | A | C |
| ATOM | 2538 | CB | GLU | A | 575 | 4.131 | 9.120 | −3.949 | 1.00 | 44.66 | A | C |
| ATOM | 2541 | CG | GLU | A | 575 | 4.611 | 8.287 | −5.139 | 1.00 | 45.56 | A | C |
| ATOM | 2544 | CD | GLU | A | 575 | 5.484 | 9.057 | −6.128 | 1.00 | 47.20 | A | C |
| ATOM | 2545 | OE1 | GLU | A | 575 | 6.216 | 9.984 | −5.710 | 1.00 | 47.38 | A | O |
| ATOM | 2546 | OE2 | GLU | A | 575 | 5.450 | 8.721 | −7.335 | 1.00 | 47.58 | A | O |
| ATOM | 2547 | C | GLU | A | 575 | 3.162 | 7.383 | −2.392 | 1.00 | 42.62 | A | C |
| ATOM | 2548 | O | GLU | A | 575 | 2.802 | 6.252 | −2.726 | 1.00 | 43.25 | A | O |
| ATOM | 2549 | N | ASP | A | 576 | 3.840 | 7.644 | −1.266 | 1.00 | 40.22 | A | N |
| ATOM | 2551 | CA | ASP | A | 576 | 4.075 | 6.649 | −0.202 | 1.00 | 37.98 | A | C |
| ATOM | 2553 | CB | ASP | A | 576 | 2.750 | 5.995 | 0.225 | 1.00 | 38.18 | A | C |
| ATOM | 2556 | CG | ASP | A | 576 | 2.910 | 5.016 | 1.364 | 1.00 | 39.08 | A | C |
| ATOM | 2557 | OD1 | ASP | A | 576 | 2.937 | 3.796 | 1.076 | 1.00 | 38.78 | A | O |
| ATOM | 2558 | OD2 | ASP | A | 576 | 2.985 | 5.364 | 2.570 | 1.00 | 41.48 | A | O |
| ATOM | 2559 | C | ASP | A | 576 | 5.106 | 5.614 | −0.658 | 1.00 | 35.29 | A | C |
| ATOM | 2560 | O | ASP | A | 576 | 5.117 | 5.222 | −1.818 | 1.00 | 35.14 | A | O |
| ATOM | 2561 | N | GLU | A | 577 | 5.977 | 5.182 | 0.251 | 1.00 | 32.19 | A | N |
| ATOM | 2563 | CA | GLU | A | 577 | 7.146 | 4.377 | −0.133 | 1.00 | 29.90 | A | C |
| ATOM | 2565 | CB | GLU | A | 577 | 8.307 | 4.597 | 0.858 | 1.00 | 29.98 | A | C |
| ATOM | 2568 | CG | GLU | A | 577 | 8.924 | 5.992 | 0.745 | 1.00 | 29.86 | A | C |
| ATOM | 2571 | CD | GLU | A | 577 | 10.252 | 6.149 | 1.479 | 1.00 | 30.30 | A | C |
| ATOM | 2572 | OE1 | GLU | A | 577 | 11.230 | 5.443 | 1.141 | 1.00 | 27.60 | A | O |
| ATOM | 2573 | OE2 | GLU | A | 577 | 10.322 | 7.008 | 2.384 | 1.00 | 30.64 | A | O |
| ATOM | 2574 | C | GLU | A | 577 | 6.838 | 2.878 | −0.335 | 1.00 | 27.95 | A | C |
| ATOM | 2575 | O | GLU | A | 577 | 7.741 | 2.092 | −0.617 | 1.00 | 26.38 | A | O |
| ATOM | 2576 | N | ASP | A | 578 | 5.568 | 2.488 | −0.214 | 1.00 | 25.79 | A | N |
| ATOM | 2578 | CA | ASP | A | 578 | 5.152 | 1.130 | −0.565 | 1.00 | 24.73 | A | C |
| ATOM | 2580 | CB | ASP | A | 578 | 3.756 | 0.802 | −0.009 | 1.00 | 24.59 | A | C |
| ATOM | 2583 | CG | ASP | A | 578 | 3.755 | 0.507 | 1.479 | 1.00 | 24.22 | A | C |
| ATOM | 2584 | OD1 | ASP | A | 578 | 4.758 | 0.759 | 2.173 | 1.00 | 22.79 | A | O |
| ATOM | 2585 | OD2 | ASP | A | 578 | 2.761 | 0.021 | 2.040 | 1.00 | 23.67 | A | O |
| ATOM | 2586 | C | ASP | A | 578 | 5.113 | 0.914 | −2.085 | 1.00 | 24.30 | A | C |
| ATOM | 2587 | O | ASP | A | 578 | 5.010 | −0.223 | −2.538 | 1.00 | 22.85 | A | O |
| ATOM | 2588 | N | TYR | A | 579 | 5.172 | 2.001 | −2.859 | 1.00 | 24.45 | A | N |
| ATOM | 2590 | CA | TYR | A | 579 | 5.029 | 1.934 | −4.315 | 1.00 | 25.01 | A | C |
| ATOM | 2592 | CB | TYR | A | 579 | 3.928 | 2.908 | −4.770 | 1.00 | 25.04 | A | C |
| ATOM | 2595 | CG | TYR | A | 579 | 2.556 | 2.582 | −4.218 | 1.00 | 24.47 | A | C |
| ATOM | 2596 | CD1 | TYR | A | 579 | 2.195 | 2.963 | −2.928 | 1.00 | 25.69 | A | C |
| ATOM | 2598 | CE1 | TYR | A | 579 | 0.932 | 2.665 | −2.409 | 1.00 | 25.75 | A | C |
| ATOM | 2600 | CZ | TYR | A | 579 | 0.022 | 1.977 | −3.187 | 1.00 | 25.29 | A | C |
| ATOM | 2601 | OH | TYR | A | 579 | −1.217 | 1.675 | −2.675 | 1.00 | 25.28 | A | O |
| ATOM | 2603 | CE2 | TYR | A | 579 | 0.358 | 1.577 | −4.475 | 1.00 | 25.54 | A | C |
| ATOM | 2605 | CD2 | TYR | A | 579 | 1.620 | 1.886 | −4.984 | 1.00 | 26.01 | A | C |
| ATOM | 2607 | C | TYR | A | 579 | 6.329 | 2.209 | −5.093 | 1.00 | 25.81 | A | C |
| ATOM | 2608 | O | TYR | A | 579 | 6.401 | 1.925 | −6.285 | 1.00 | 26.17 | A | O |
| ATOM | 2609 | N | TYR | A | 580 | 7.349 | 2.751 | −4.435 | 1.00 | 26.41 | A | N |
| ATOM | 2611 | CA | TYR | A | 580 | 8.617 | 3.046 | −5.106 | 1.00 | 26.93 | A | C |
| ATOM | 2613 | CB | TYR | A | 580 | 8.598 | 4.475 | −5.677 | 1.00 | 27.09 | A | C |
| ATOM | 2616 | CG | TYR | A | 580 | 8.584 | 5.554 | −4.622 | 1.00 | 26.95 | A | C |
| ATOM | 2617 | CD1 | TYR | A | 580 | 9.771 | 6.087 | −4.123 | 1.00 | 27.01 | A | C |
| ATOM | 2619 | CE1 | TYR | A | 580 | 9.764 | 7.069 | −3.135 | 1.00 | 27.38 | A | C |
| ATOM | 2621 | CZ | TYR | A | 580 | 8.558 | 7.541 | −2.647 | 1.00 | 28.47 | A | C |
| ATOM | 2622 | OH | TYR | A | 580 | 8.545 | 8.516 | −1.674 | 1.00 | 29.60 | A | O |
| ATOM | 2624 | CE2 | TYR | A | 580 | 7.363 | 7.032 | −3.132 | 1.00 | 28.07 | A | C |
| ATOM | 2626 | CD2 | TYR | A | 580 | 7.381 | 6.043 | −4.113 | 1.00 | 27.45 | A | C |
| ATOM | 2628 | C | TYR | A | 580 | 9.823 | 2.867 | −4.183 | 1.00 | 27.08 | A | C |
| ATOM | 2629 | O | TYR | A | 580 | 9.705 | 2.962 | −2.949 | 1.00 | 27.01 | A | O |
| ATOM | 2630 | N | LYS | A | 581 | 10.981 | 2.607 | −4.787 | 1.00 | 27.09 | A | N |
| ATOM | 2632 | CA | LYS | A | 581 | 12.230 | 2.554 | −4.044 | 1.00 | 27.49 | A | C |
| ATOM | 2634 | CB | LYS | A | 581 | 13.167 | 1.469 | −4.591 | 1.00 | 27.82 | A | C |
| ATOM | 2637 | CG | LYS | A | 581 | 12.624 | 0.066 | −4.493 | 1.00 | 29.08 | A | C |
| ATOM | 2640 | CD | LYS | A | 581 | 12.553 | −0.410 | −3.054 | 1.00 | 29.95 | A | C |
| ATOM | 2643 | CE | LYS | A | 581 | 12.586 | −1.923 | −2.981 | 1.00 | 30.16 | A | C |
| ATOM | 2646 | NZ | LYS | A | 581 | 13.933 | −2.487 | −3.300 | 1.00 | 31.13 | A | N |
| ATOM | 2650 | C | LYS | A | 581 | 12.913 | 3.910 | −4.147 | 1.00 | 27.35 | A | C |
| ATOM | 2651 | O | LYS | A | 581 | 13.313 | 4.327 | −5.244 | 1.00 | 27.59 | A | O |
| ATOM | 2652 | N | ALA | A | 582 | 13.045 | 4.587 | −3.008 | 1.00 | 26.87 | A | N |
| ATOM | 2654 | CA | ALA | A | 582 | 13.721 | 5.877 | −2.940 | 1.00 | 27.05 | A | C |
| ATOM | 2656 | CB | ALA | A | 582 | 13.460 | 6.524 | −1.587 | 1.00 | 27.03 | A | C |
| ATOM | 2660 | C | ALA | A | 582 | 15.219 | 5.686 | −3.147 | 1.00 | 27.17 | A | C |
| ATOM | 2661 | O | ALA | A | 582 | 15.770 | 4.669 | −2.733 | 1.00 | 26.90 | A | O |
| ATOM | 2662 | N | SER | A | 583 | 15.875 | 6.657 | −3.789 | 1.00 | 26.98 | A | N |
| ATOM | 2664 | CA | SER | A | 583 | 17.342 | 6.691 | −3.818 | 1.00 | 27.27 | A | C |
| ATOM | 2666 | CB | SER | A | 583 | 17.864 | 7.934 | −4.568 | 1.00 | 27.02 | A | C |
| ATOM | 2669 | OG | SER | A | 583 | 17.666 | 7.842 | −5.971 | 1.00 | 24.95 | A | O |
| ATOM | 2671 | C | SER | A | 583 | 17.899 | 6.673 | −2.390 | 1.00 | 27.51 | A | C |
| ATOM | 2672 | O | SER | A | 583 | 18.853 | 5.960 | −2.111 | 1.00 | 28.19 | A | O |
| ATOM | 2673 | N | VAL | A | 584 | 17.301 | 7.481 | −1.511 | 1.00 | 27.95 | A | N |
| ATOM | 2675 | CA | VAL | A | 584 | 17.624 | 7.547 | −0.081 | 1.00 | 27.95 | A | C |
| ATOM | 2677 | CB | VAL | A | 584 | 18.359 | 8.860 | 0.279 | 1.00 | 28.18 | A | C |
| ATOM | 2679 | CG1 | VAL | A | 584 | 18.684 | 8.910 | 1.762 | 1.00 | 29.19 | A | C |
| ATOM | 2683 | CG2 | VAL | A | 584 | 19.610 | 8.997 | −0.529 | 1.00 | 28.53 | A | C |
| ATOM | 2687 | C | VAL | A | 584 | 16.314 | 7.499 | 0.716 | 1.00 | 27.93 | A | C |
| ATOM | 2688 | O | VAL | A | 584 | 15.532 | 8.445 | 0.693 | 1.00 | 27.03 | A | O |
| ATOM | 2689 | N | THR | A | 585 | 16.072 | 6.403 | 1.428 | 1.00 | 28.20 | A | N |
| ATOM | 2691 | CA | THR | A | 585 | 14.795 | 6.239 | 2.111 | 1.00 | 28.72 | A | C |
| ATOM | 2693 | CB | THR | A | 585 | 14.394 | 4.744 | 2.181 | 1.00 | 28.79 | A | C |
| ATOM | 2695 | CG1 | THR | A | 585 | 13.033 | 4.623 | 2.621 | 1.00 | 29.29 | A | O |
| ATOM | 2697 | CG2 | THR | A | 585 | 15.193 | 3.992 | 3.216 | 1.00 | 29.13 | A | C |
| ATOM | 2701 | C | THR | A | 585 | 14.779 | 6.882 | 3.494 | 1.00 | 28.58 | A | C |
| ATOM | 2702 | O | THR | A | 585 | 15.809 | 7.081 | 4.129 | 1.00 | 29.24 | A | O |
| ATOM | 2703 | N | ARG | A | 586 | 13.572 | 7.185 | 3.951 | 1.00 | 28.53 | A | N |
| ATOM | 2705 | CA | ARG | A | 586 | 13.331 | 7.764 | 5.266 | 1.00 | 27.88 | A | C |
| ATOM | 2707 | CB | ARG | A | 586 | 12.163 | 8.751 | 5.159 | 1.00 | 28.97 | A | C |
| ATOM | 2710 | CG | ARG | A | 586 | 12.163 | 9.613 | 3.892 | 1.00 | 32.87 | A | C |
| ATOM | 2713 | CD | ARG | A | 586 | 11.091 | 10.706 | 3.880 | 1.00 | 37.35 | A | C |
| ATOM | 2716 | NE | ARG | A | 586 | 11.446 | 11.869 | 4.700 | 1.00 | 40.84 | A | N |
| ATOM | 2718 | CZ | ARG | A | 586 | 12.287 | 12.850 | 4.340 | 1.00 | 42.20 | A | C |
| ATOM | 2719 | NH1 | ARG | A | 586 | 12.897 | 12.839 | 3.157 | 1.00 | 42.91 | A | N |
| ATOM | 2722 | NH2 | ARG | A | 586 | 12.524 | 13.854 | 5.183 | 1.00 | 42.67 | A | N |
| ATOM | 2725 | C | ARG | A | 586 | 12.978 | 6.639 | 6.271 | 1.00 | 25.82 | A | C |
| ATOM | 2726 | O | ARG | A | 586 | 12.956 | 6.850 | 7.485 | 1.00 | 25.52 | A | O |
| ATOM | 2727 | N | LEU | A | 587 | 12.724 | 5.449 | 5.742 | 1.00 | 23.19 | A | N |
| ATOM | 2729 | CA | LEU | A | 587 | 12.216 | 4.320 | 6.520 | 1.00 | 21.02 | A | C |
| ATOM | 2731 | CB | LEU | A | 587 | 11.740 | 3.228 | 5.569 | 1.00 | 21.26 | A | C |
| ATOM | 2734 | CG | LEU | A | 587 | 10.591 | 3.508 | 4.618 | 1.00 | 22.52 | A | C |
| ATOM | 2736 | CD1 | LEU | A | 587 | 10.428 | 2.299 | 3.713 | 1.00 | 23.24 | A | C |
| ATOM | 2740 | CD2 | LEU | A | 587 | 9.314 | 3.812 | 5.391 | 1.00 | 23.89 | A | C |
| ATOM | 2744 | C | LEU | A | 587 | 13.303 | 3.726 | 7.410 | 1.00 | 19.25 | A | C |
| ATOM | 2745 | O | LEU | A | 587 | 14.475 | 3.822 | 7.075 | 1.00 | 18.79 | A | O |
| ATOM | 2746 | N | PRO | A | 588 | 12.911 | 3.068 | 8.506 | 1.00 | 16.35 | A | N |
| ATOM | 2747 | CA | PRO | A | 588 | 13.877 | 2.494 | 9.451 | 1.00 | 14.93 | A | C |
| ATOM | 2749 | CB | PRO | A | 588 | 13.037 | 2.286 | 10.717 | 1.00 | 14.98 | A | C |
| ATOM | 2752 | CG | PRO | A | 588 | 11.683 | 1.973 | 10.177 | 1.00 | 15.18 | A | C |
| ATOM | 2755 | CD | PRO | A | 588 | 11.513 | 2.828 | 8.927 | 1.00 | 16.26 | A | C |
| ATOM | 2758 | C | PRO | A | 588 | 14.465 | 1.175 | 8.947 | 1.00 | 13.29 | A | C |
| ATOM | 2759 | O | PRO | A | 588 | 14.183 | 0.083 | 9.484 | 1.00 | 12.58 | A | O |
| ATOM | 2760 | N | ILE | A | 589 | 15.309 | 1.281 | 7.925 | 1.00 | 11.92 | A | N |
| ATOM | 2762 | CA | ILE | A | 589 | 15.866 | 0.118 | 7.230 | 1.00 | 11.54 | A | C |
| ATOM | 2764 | CB | ILE | A | 589 | 16.889 | 0.600 | 6.152 | 1.00 | 11.43 | A | C |
| ATOM | 2766 | CG1 | ILE | A | 589 | 16.174 | 1.366 | 5.024 | 1.00 | 13.60 | A | C |
| ATOM | 2769 | CD1 | ILE | A | 589 | 15.092 | 0.544 | 4.305 | 1.00 | 14.47 | A | C |
| ATOM | 2773 | CG2 | ILE | A | 589 | 17.700 | −0.581 | 5.601 | 1.00 | 12.99 | A | C |
| ATOM | 2777 | C | ILE | A | 589 | 16.574 | −0.864 | 8.203 | 1.00 | 10.07 | A | C |
| ATOM | 2778 | O | ILE | A | 589 | 16.471 | −2.075 | 8.055 | 1.00 | 9.44 | A | O |
| ATOM | 2779 | N | LYS | A | 590 | 17.300 | −0.330 | 9.186 | 1.00 | 9.76 | A | N |
| ATOM | 2781 | CA | LYS | A | 590 | 18.065 | −1.186 | 10.109 | 1.00 | 9.66 | A | C |
| ATOM | 2783 | CB | LYS | A | 590 | 19.111 | −0.377 | 10.888 | 1.00 | 9.94 | A | C |
| ATOM | 2786 | CG | LYS | A | 590 | 20.252 | 0.122 | 10.016 | 1.00 | 10.30 | A | C |
| ATOM | 2789 | CD | LYS | A | 590 | 21.098 | 1.207 | 10.703 | 1.00 | 10.44 | A | C |
| ATOM | 2792 | CE | LYS | A | 590 | 22.248 | 1.579 | 9.797 | 1.00 | 12.41 | A | C |
| ATOM | 2795 | NZ | LYS | A | 590 | 23.160 | 2.618 | 10.380 | 1.00 | 12.07 | A | N |
| ATOM | 2799 | C | LYS | A | 590 | 17.174 | −1.954 | 11.068 | 1.00 | 9.73 | A | C |
| ATOM | 2800 | O | LYS | A | 590 | 17.660 | −2.830 | 11.790 | 1.00 | 9.50 | A | O |
| ATOM | 2801 | N | TRP | A | 591 | 15.876 | −1.628 | 11.089 | 1.00 | 9.17 | A | N |
| ATOM | 2803 | CA | TRP | A | 591 | 14.905 | −2.339 | 11.912 | 1.00 | 9.87 | A | C |
| ATOM | 2805 | CB | TRP | A | 591 | 14.017 | −1.349 | 12.662 | 1.00 | 9.98 | A | C |
| ATOM | 2808 | CG | TRP | A | 591 | 14.655 | −0.542 | 13.766 | 1.00 | 11.71 | A | C |
| ATOM | 2809 | CD1 | TRP | A | 591 | 14.457 | −0.709 | 15.103 | 1.00 | 12.20 | A | C |
| ATOM | 2811 | NE1 | TRP | A | 591 | 15.141 | 0.252 | 15.803 | 1.00 | 13.37 | A | N |
| ATOM | 2813 | CE2 | TRP | A | 591 | 15.793 | 1.065 | 14.924 | 1.00 | 12.15 | A | C |
| ATOM | 2814 | CD2 | TRP | A | 591 | 15.490 | 0.607 | 13.627 | 1.00 | 13.10 | A | C |
| ATOM | 2815 | CE3 | TRP | A | 591 | 16.031 | 1.299 | 12.533 | 1.00 | 12.17 | A | C |
| ATOM | 2817 | CZ3 | TRP | A | 591 | 16.853 | 2.386 | 12.772 | 1.00 | 15.31 | A | C |
| ATOM | 2819 | CH2 | TRP | A | 591 | 17.127 | 2.813 | 14.082 | 1.00 | 13.32 | A | C |
| ATOM | 2821 | CZ2 | TRP | A | 591 | 16.621 | 2.156 | 15.161 | 1.00 | 12.48 | A | C |
| ATOM | 2823 | C | TRP | A | 591 | 13.997 | −3.282 | 11.108 | 1.00 | 9.92 | A | C |
| ATOM | 2824 | O | TRP | A | 591 | 13.223 | −4.039 | 11.706 | 1.00 | 10.09 | A | O |
| ATOM | 2825 | N | MET | A | 592 | 14.096 | −3.251 | 9.773 | 1.00 | 9.75 | A | N |
| ATOM | 2827 | CA | MET | A | 592 | 13.094 | −3.843 | 8.881 | 1.00 | 9.99 | A | C |
| ATOM | 2829 | CB | MET | A | 592 | 12.876 | −2.908 | 7.667 | 1.00 | 9.83 | A | C |
| ATOM | 2832 | CG | MET | A | 592 | 12.051 | −1.676 | 7.999 | 1.00 | 11.13 | A | C |
| ATOM | 2835 | SD | MET | A | 592 | 12.111 | −0.339 | 6.763 | 1.00 | 14.42 | A | S |
| ATOM | 2836 | CE | MET | A | 592 | 11.786 | −1.271 | 5.270 | 1.00 | 11.86 | A | C |
| ATOM | 2840 | C | MET | A | 592 | 13.432 | −5.244 | 8.373 | 1.00 | 9.53 | A | C |
| ATOM | 2841 | O | MET | A | 592 | 14.602 | −5.589 | 8.192 | 1.00 | 10.45 | A | O |
| ATOM | 2842 | N | SER | A | 593 | 12.395 | −6.034 | 8.118 | 1.00 | 9.01 | A | N |
| ATOM | 2844 | CA | SER | A | 593 | 12.558 | −7.391 | 7.605 | 1.00 | 9.63 | A | C |
| ATOM | 2846 | CB | SER | A | 593 | 11.241 | −8.149 | 7.607 | 1.00 | 10.07 | A | C |
| ATOM | 2849 | OG | SER | A | 593 | 10.406 | −7.605 | 6.617 | 1.00 | 10.36 | A | O |
| ATOM | 2851 | C | SER | A | 593 | 13.073 | −7.313 | 6.164 | 1.00 | 9.56 | A | C |
| ATOM | 2852 | O | SER | A | 593 | 12.921 | −6.287 | 5.511 | 1.00 | 10.05 | A | O |
| ATOM | 2853 | N | PRO | A | 594 | 13.695 | −8.378 | 5.686 | 1.00 | 10.88 | A | N |
| ATOM | 2854 | CA | PRO | A | 594 | 14.197 | −8.420 | 4.301 | 1.00 | 11.51 | A | C |
| ATOM | 2856 | CB | PRO | A | 594 | 14.787 | −9.828 | 4.191 | 1.00 | 12.13 | A | C |
| ATOM | 2859 | CG | PRO | A | 594 | 15.141 | −10.176 | 5.577 | 1.00 | 12.75 | A | C |
| ATOM | 2862 | CD | PRO | A | 594 | 14.022 | −9.610 | 6.420 | 1.00 | 10.32 | A | C |
| ATOM | 2865 | C | PRO | A | 594 | 13.108 | −8.199 | 3.254 | 1.00 | 11.67 | A | C |
| ATOM | 2866 | O | PRO | A | 594 | 13.366 | −7.517 | 2.289 | 1.00 | 12.87 | A | O |
| ATOM | 2867 | N | GLU | A | 595 | 11.916 | −8.754 | 3.455 | 1.00 | 12.18 | A | N |
| ATOM | 2869 | CA | GLU | A | 595 | 10.819 | −8.588 | 2.509 | 1.00 | 11.54 | A | C |
| ATOM | 2871 | CB | GLU | A | 595 | 9.685 | −9.578 | 2.796 | 1.00 | 11.95 | A | C |
| ATOM | 2874 | CG | GLU | A | 595 | 8.905 | −9.349 | 4.080 | 1.00 | 12.54 | A | C |
| ATOM | 2877 | CD | GLU | A | 595 | 9.410 | −10.169 | 5.261 | 1.00 | 12.35 | A | C |
| ATOM | 2878 | OE1 | GLU | A | 595 | 10.587 | −10.628 | 5.259 | 1.00 | 12.67 | A | O |
| ATOM | 2879 | OE2 | GLU | A | 595 | 8.624 | −10.313 | 6.232 | 1.00 | 11.80 | A | O |
| ATOM | 2880 | C | GLU | A | 595 | 10.325 | −7.139 | 2.494 | 1.00 | 11.35 | A | C |
| ATOM | 2881 | O | GLU | A | 595 | 9.870 | −6.621 | 1.462 | 1.00 | 10.36 | A | O |
| ATOM | 2882 | N | SER | A | 596 | 10.453 | −6.468 | 3.639 | 1.00 | 11.09 | A | N |
| ATOM | 2884 | CA | SER | A | 596 | 10.129 | −5.054 | 3.746 | 1.00 | 11.11 | A | C |
| ATOM | 2886 | CB | SER | A | 596 | 10.035 | −4.639 | 5.215 | 1.00 | 11.61 | A | C |
| ATOM | 2889 | OG | SER | A | 596 | 9.071 | −5.412 | 5.916 | 1.00 | 10.96 | A | O |
| ATOM | 2891 | C | SER | A | 596 | 11.154 | −4.165 | 3.009 | 1.00 | 11.46 | A | C |
| ATOM | 2892 | O | SER | A | 596 | 10.780 | −3.187 | 2.349 | 1.00 | 11.08 | A | O |
| ATOM | 2893 | N | ILE | A | 597 | 12.433 | −4.494 | 3.154 | 1.00 | 11.82 | A | N |
| ATOM | 2895 | CA | ILE | A | 597 | 13.498 | −3.793 | 2.447 | 1.00 | 12.20 | A | C |
| ATOM | 2897 | CB | ILE | A | 597 | 14.870 | −4.196 | 3.012 | 1.00 | 12.29 | A | C |
| ATOM | 2899 | CG1 | ILE | A | 597 | 15.044 | −3.734 | 4.479 | 1.00 | 11.10 | A | C |
| ATOM | 2902 | CD1 | ILE | A | 597 | 16.321 | −4.267 | 5.140 | 1.00 | 10.75 | A | C |
| ATOM | 2906 | CG2 | ILE | A | 597 | 15.980 | −3.586 | 2.160 | 1.00 | 13.21 | A | C |
| ATOM | 2910 | C | ILE | A | 597 | 13.433 | −4.051 | 0.923 | 1.00 | 12.61 | A | C |
| ATOM | 2911 | O | ILE | A | 597 | 13.521 | −3.120 | 0.135 | 1.00 | 14.25 | A | O |
| ATOM | 2912 | N | ASN | A | 598 | 13.275 | −5.298 | 0.514 | 1.00 | 12.98 | A | N |
| ATOM | 2914 | CA | ASN | A | 598 | 13.331 | −5.672 | −0.919 | 1.00 | 14.27 | A | C |
| ATOM | 2916 | CB | ASN | A | 598 | 13.618 | −7.179 | −1.052 | 1.00 | 14.04 | A | C |
| ATOM | 2919 | CG | ASN | A | 598 | 15.068 | −7.523 | −0.790 | 1.00 | 16.01 | A | C |
| ATOM | 2920 | OD1 | ASN | A | 598 | 15.971 | −6.727 | −1.066 | 1.00 | 18.13 | A | O |
| ATOM | 2921 | ND2 | ASN | A | 598 | 15.307 | −8.734 | −0.283 | 1.00 | 16.92 | A | N |
| ATOM | 2924 | C | ASN | A | 598 | 12.077 | −5.337 | −1.725 | 1.00 | 15.32 | A | C |
| ATOM | 2925 | O | ASN | A | 598 | 12.162 | −4.886 | −2.877 | 1.00 | 15.17 | A | O |
| ATOM | 2926 | N | PHE | A | 599 | 10.912 | −5.552 | −1.119 | 1.00 | 15.42 | A | N |
| ATOM | 2928 | CA | PHE | A | 599 | 9.636 | −5.496 | −1.839 | 1.00 | 15.79 | A | C |
| ATOM | 2930 | CB | PHE | A | 599 | 9.088 | −6.914 | −2.039 | 1.00 | 15.78 | A | C |
| ATOM | 2933 | CG | PHE | A | 599 | 10.095 | −7.884 | −2.566 | 1.00 | 18.17 | A | C |
| ATOM | 2934 | CD1 | PHE | A | 599 | 10.746 | −7.643 | −3.769 | 1.00 | 20.18 | A | C |
| ATOM | 2936 | CE1 | PHE | A | 599 | 11.694 | −8.537 | −4.245 | 1.00 | 21.90 | A | C |
| ATOM | 2938 | CZ | PHE | A | 599 | 11.975 | −9.693 | −3.532 | 1.00 | 20.74 | A | C |
| ATOM | 2940 | CE2 | PHE | A | 599 | 11.318 | −9.945 | −2.336 | 1.00 | 21.69 | A | C |
| ATOM | 2942 | CD2 | PHE | A | 599 | 10.391 | −9.038 | −1.860 | 1.00 | 19.49 | A | C |
| ATOM | 2944 | C | PHE | A | 599 | 8.574 | −4.641 | −1.156 | 1.00 | 15.14 | A | C |
| ATOM | 2945 | O | PHE | A | 599 | 7.427 | −4.610 | −1.608 | 1.00 | 15.42 | A | O |
| ATOM | 2946 | N | ARG | A | 600 | 8.951 | −3.936 | −0.092 | 1.00 | 15.13 | A | N |
| ATOM | 2948 | CA | ARG | A | 600 | 8.024 | −3.137 | 0.701 | 1.00 | 15.85 | A | C |
| ATOM | 2950 | CB | ARG | A | 600 | 7.614 | −1.880 | −0.076 | 1.00 | 16.31 | A | C |
| ATOM | 2953 | CG | ARG | A | 600 | 8.784 | −0.950 | −0.377 | 1.00 | 17.77 | A | C |
| ATOM | 2956 | CD | ARG | A | 600 | 9.269 | −0.173 | 0.835 | 1.00 | 18.17 | A | C |
| ATOM | 2959 | NE | ARG | A | 600 | 10.355 | 0.750 | 0.508 | 1.00 | 20.72 | A | N |
| ATOM | 2961 | CZ | ARG | A | 600 | 11.657 | 0.490 | 0.633 | 1.00 | 21.90 | A | C |
| ATOM | 2962 | NH1 | ARG | A | 600 | 12.094 | −0.680 | 1.078 | 1.00 | 22.93 | A | N |
| ATOM | 2965 | NH2 | ARG | A | 600 | 12.543 | 1.419 | 0.299 | 1.00 | 22.61 | A | N |
| ATOM | 2968 | C | ARG | A | 600 | 6.793 | −3.956 | 1.149 | 1.00 | 15.58 | A | C |
| ATOM | 2969 | O | ARG | A | 600 | 5.653 | −3.464 | 1.136 | 1.00 | 15.40 | A | O |
| ATOM | 2970 | N | ARG | A | 601 | 7.046 | −5.206 | 1.535 | 1.00 | 15.64 | A | N |
| ATOM | 2972 | CA | ARG | A | 601 | 6.022 | −6.120 | 2.052 | 1.00 | 16.29 | A | C |
| ATOM | 2974 | CB | ARG | A | 601 | 6.363 | −7.569 | 1.724 | 1.00 | 17.23 | A | C |
| ATOM | 2977 | CG | ARG | A | 601 | 6.233 | −7.938 | 0.259 | 1.00 | 21.68 | A | C |
| ATOM | 2980 | CD | ARG | A | 601 | 5.955 | −9.440 | −0.022 | 1.00 | 25.56 | A | C |
| ATOM | 2983 | NE | ARG | A | 601 | 6.621 | −9.837 | −1.270 | 1.00 | 30.36 | A | N |
| ATOM | 2985 | CZ | ARG | A | 601 | 6.043 | −10.295 | −2.379 | 1.00 | 31.71 | A | C |
| ATOM | 2986 | NH1 | ARG | A | 601 | 4.738 | −10.497 | −2.462 | 1.00 | 34.89 | A | N |
| ATOM | 2989 | NH2 | ARG | A | 601 | 6.800 | −10.599 | −3.428 | 1.00 | 34.61 | A | N |
| ATOM | 2992 | C | ARG | A | 601 | 6.002 | −5.963 | 3.571 | 1.00 | 15.73 | A | C |
| ATOM | 2993 | O | ARG | A | 601 | 7.033 | −6.218 | 4.233 | 1.00 | 15.42 | A | O |
| ATOM | 2994 | N | PHE | A | 602 | 4.866 | −5.511 | 4.105 | 1.00 | 14.28 | A | N |
| ATOM | 2996 | CA | PHE | A | 602 | 4.684 | −5.323 | 5.543 | 1.00 | 14.03 | A | C |
| ATOM | 2998 | CB | PHE | A | 602 | 4.463 | −3.844 | 5.876 | 1.00 | 13.94 | A | C |
| ATOM | 3001 | CG | PHE | A | 602 | 5.599 | −2.947 | 5.501 | 1.00 | 15.36 | A | C |
| ATOM | 3002 | CD1 | PHE | A | 602 | 6.596 | −2.632 | 6.430 | 1.00 | 16.03 | A | C |
| ATOM | 3004 | CE1 | PHE | A | 602 | 7.651 | −1.772 | 6.090 | 1.00 | 15.79 | A | C |
| ATOM | 3006 | CZ | PHE | A | 602 | 7.690 | −1.198 | 4.833 | 1.00 | 15.40 | A | C |
| ATOM | 3008 | CE2 | PHE | A | 602 | 6.686 | −1.485 | 3.909 | 1.00 | 15.83 | A | C |
| ATOM | 3010 | CD2 | PHE | A | 602 | 5.643 | −2.348 | 4.245 | 1.00 | 14.53 | A | C |
| ATOM | 3012 | C | PHE | A | 602 | 3.499 | −6.130 | 6.054 | 1.00 | 13.39 | A | C |
| ATOM | 3013 | O | PHE | A | 602 | 2.353 | −5.927 | 5.631 | 1.00 | 13.20 | A | O |
| ATOM | 3014 | N | THR | A | 603 | 3.766 | −7.070 | 6.947 | 1.00 | 13.12 | A | N |
| ATOM | 3016 | CA | THR | A | 603 | 2.739 | −7.941 | 7.518 | 1.00 | 12.82 | A | C |
| ATOM | 3018 | CB | THR | A | 603 | 2.851 | −9.331 | 6.902 | 1.00 | 13.20 | A | C |
| ATOM | 3020 | OG1 | THR | A | 603 | 4.143 | −9.881 | 7.212 | 1.00 | 13.90 | A | O |
| ATOM | 3022 | CG2 | THR | A | 603 | 2.771 | −9.277 | 5.357 | 1.00 | 14.67 | A | C |
| ATOM | 3026 | C | THR | A | 603 | 2.968 | −8.095 | 9.007 | 1.00 | 12.15 | A | C |
| ATOM | 3027 | O | THR | A | 603 | 3.936 | −7.588 | 9.552 | 1.00 | 10.36 | A | O |
| ATOM | 3028 | N | THR | A | 604 | 2.125 | −8.874 | 9.659 | 1.00 | 11.47 | A | N |
| ATOM | 3030 | CA | THR | A | 604 | 2.387 | −9.169 | 11.053 | 1.00 | 11.50 | A | C |
| ATOM | 3032 | CB | THR | A | 604 | 1.228 | −9.955 | 11.676 | 1.00 | 12.26 | A | C |
| ATOM | 3034 | OG1 | THR | A | 604 | 0.043 | −9.138 | 11.648 | 1.00 | 14.28 | A | O |
| ATOM | 3036 | CG2 | THR | A | 604 | 1.493 | −10.182 | 13.155 | 1.00 | 14.14 | A | C |
| ATOM | 3040 | C | THR | A | 604 | 3.728 | −9.884 | 11.203 | 1.00 | 11.13 | A | C |
| ATOM | 3041 | O | THR | A | 604 | 4.391 | −9.699 | 12.211 | 1.00 | 9.85 | A | O |
| ATOM | 3042 | N | ALA | A | 605 | 4.142 | −10.679 | 10.206 | 1.00 | 10.23 | A | N |
| ATOM | 3044 | CA | ALA | A | 605 | 5.450 | −11.338 | 10.258 | 1.00 | 10.32 | A | C |
| ATOM | 3046 | CB | ALA | A | 605 | 5.608 | −12.374 | 9.156 | 1.00 | 10.78 | A | C |
| ATOM | 3050 | C | ALA | A | 605 | 6.611 | −10.363 | 10.197 | 1.00 | 9.56 | A | C |
| ATOM | 3051 | O | ALA | A | 605 | 7.640 | −10.616 | 10.824 | 1.00 | 8.50 | A | O |
| ATOM | 3052 | N | SER | A | 606 | 6.459 | −9.272 | 9.445 | 1.00 | 8.64 | A | N |
| ATOM | 3054 | CA | SER | A | 606 | 7.486 | −8.245 | 9.412 | 1.00 | 9.24 | A | C |
| ATOM | 3056 | CB | SER | A | 606 | 7.352 | −7.296 | 8.190 | 1.00 | 9.76 | A | C |
| ATOM | 3059 | OG | SER | A | 606 | 6.179 | −6.496 | 8.250 | 1.00 | 10.66 | A | O |
| ATOM | 3061 | C | SER | A | 606 | 7.505 | −7.489 | 10.748 | 1.00 | 9.16 | A | C |
| ATOM | 3062 | O | SER | A | 606 | 8.572 | −7.100 | 11.211 | 1.00 | 8.11 | A | O |
| ATOM | 3063 | N | ASP | A | 607 | 6.340 | −7.307 | 11.379 | 1.00 | 8.37 | A | N |
| ATOM | 3065 | CA | ASP | A | 607 | 6.297 | −6.717 | 12.720 | 1.00 | 8.68 | A | C |
| ATOM | 3067 | CB | ASP | A | 607 | 4.876 | −6.507 | 13.225 | 1.00 | 8.94 | A | C |
| ATOM | 3070 | CG | ASP | A | 607 | 4.256 | −5.197 | 12.785 | 1.00 | 11.42 | A | C |
| ATOM | 3071 | OD1 | ASP | A | 607 | 4.925 | −4.280 | 12.218 | 1.00 | 11.99 | A | O |
| ATOM | 3072 | OD2 | ASP | A | 607 | 3.029 | −5.001 | 13.022 | 1.00 | 12.74 | A | O |
| ATOM | 3073 | C | ASP | A | 607 | 7.034 | −7.616 | 13.740 | 1.00 | 8.12 | A | C |
| ATOM | 3074 | O | ASP | A | 607 | 7.628 | −7.111 | 14.678 | 1.00 | 7.11 | A | O |
| ATOM | 3075 | N | VAL | A | 608 | 6.952 | −8.940 | 13.577 | 1.00 | 7.25 | A | N |
| ATOM | 3077 | CA | VAL | A | 608 | 7.677 | −9.870 | 14.461 | 1.00 | 7.14 | A | C |
| ATOM | 3079 | CB | VAL | A | 608 | 7.280 | −11.342 | 14.171 | 1.00 | 6.69 | A | C |
| ATOM | 3081 | CG1 | VAL | A | 608 | 8.277 | −12.331 | 14.822 | 1.00 | 8.13 | A | C |
| ATOM | 3085 | CG2 | VAL | A | 608 | 5.840 | −11.616 | 14.668 | 1.00 | 7.95 | A | C |
| ATOM | 3089 | C | VAL | A | 608 | 9.187 | −9.687 | 14.332 | 1.00 | 7.61 | A | C |
| ATOM | 3090 | O | VAL | A | 608 | 9.902 | −9.615 | 15.345 | 1.00 | 6.65 | A | O |
| ATOM | 3091 | N | TRP | A | 609 | 9.673 | −9.587 | 13.090 | 1.00 | 7.07 | A | N |
| ATOM | 3093 | CA | TRP | A | 609 | 11.085 | −9.274 | 12.835 | 1.00 | 7.28 | A | C |
| ATOM | 3095 | CB | TRP | A | 609 | 11.349 | −9.095 | 11.329 | 1.00 | 7.08 | A | C |
| ATOM | 3098 | CG | TRP | A | 609 | 12.771 | −8.707 | 10.985 | 1.00 | 7.95 | A | C |
| ATOM | 3099 | CD1 | TRP | A | 609 | 13.381 | −7.506 | 11.228 | 1.00 | 7.59 | A | C |
| ATOM | 3101 | NE1 | TRP | A | 609 | 14.692 | −7.555 | 10.821 | 1.00 | 8.83 | A | N |
| ATOM | 3103 | CE2 | TRP | A | 609 | 14.958 | −8.799 | 10.310 | 1.00 | 8.13 | A | C |
| ATOM | 3104 | CD2 | TRP | A | 609 | 13.778 | −9.555 | 10.414 | 1.00 | 6.30 | A | C |
| ATOM | 3105 | CE3 | TRP | A | 609 | 13.779 | −10.881 | 9.934 | 1.00 | 6.84 | A | C |
| ATOM | 3107 | CZ3 | TRP | A | 609 | 14.944 | −11.401 | 9.398 | 1.00 | 8.38 | A | C |
| ATOM | 3109 | CH2 | TRP | A | 609 | 16.108 | −10.624 | 9.313 | 1.00 | 7.22 | A | C |
| ATOM | 3111 | CZ2 | TRP | A | 609 | 16.135 | −9.313 | 9.749 | 1.00 | 8.92 | A | C |
| ATOM | 3113 | C | TRP | A | 609 | 11.484 | −7.994 | 13.602 | 1.00 | 7.00 | A | C |
| ATOM | 3114 | O | TRP | A | 609 | 12.484 | −7.969 | 14.329 | 1.00 | 7.89 | A | O |
| ATOM | 3115 | N | MET | A | 610 | 10.687 | −6.954 | 13.449 | 1.00 | 7.31 | A | N |
| ATOM | 3117 | CA | MET | A | 610 | 11.019 | −5.634 | 13.979 | 1.00 | 7.12 | A | C |
| ATOM | 3119 | CB | MET | A | 610 | 10.083 | −4.569 | 13.380 | 1.00 | 7.25 | A | C |
| ATOM | 3122 | CG | MET | A | 610 | 10.433 | −3.155 | 13.777 | 1.00 | 8.77 | A | C |
| ATOM | 3125 | SD | MET | A | 610 | 9.336 | −1.944 | 13.037 | 1.00 | 11.18 | A | S |
| ATOM | 3126 | CE | MET | A | 610 | 10.448 | −0.489 | 13.042 | 1.00 | 12.69 | A | C |
| ATOM | 3130 | C | MET | A | 610 | 10.945 | −5.628 | 15.504 | 1.00 | 7.16 | A | C |
| ATOM | 3131 | O | MET | A | 610 | 11.740 | −4.979 | 16.171 | 1.00 | 7.31 | A | O |
| ATOM | 3132 | N | PHE | A | 611 | 9.995 | −6.376 | 16.053 | 1.00 | 7.20 | A | N |
| ATOM | 3134 | CA | PHE | A | 611 | 9.859 | −6.506 | 17.503 | 1.00 | 6.98 | A | C |
| ATOM | 3136 | CB | PHE | A | 611 | 8.614 | −7.289 | 17.857 | 1.00 | 7.02 | A | C |
| ATOM | 3139 | CG | PHE | A | 611 | 8.576 | −7.724 | 19.296 | 1.00 | 8.33 | A | C |
| ATOM | 3140 | CD1 | PHE | A | 611 | 8.329 | −6.808 | 20.294 | 1.00 | 9.98 | A | C |
| ATOM | 3142 | CE1 | PHE | A | 611 | 8.322 | −7.188 | 21.610 | 1.00 | 9.82 | A | C |
| ATOM | 3144 | CZ | PHE | A | 611 | 8.587 | −8.482 | 21.958 | 1.00 | 9.72 | A | C |
| ATOM | 3146 | CE2 | PHE | A | 611 | 8.835 | −9.415 | 20.984 | 1.00 | 11.54 | A | C |
| ATOM | 3148 | CD2 | PHE | A | 611 | 8.838 | −9.033 | 19.645 | 1.00 | 9.39 | A | C |
| ATOM | 3150 | C | PHE | A | 611 | 11.103 | −7.142 | 18.153 | 1.00 | 6.73 | A | C |
| ATOM | 3151 | O | PHE | A | 611 | 11.557 | −6.708 | 19.202 | 1.00 | 7.59 | A | O |
| ATOM | 3152 | N | ALA | A | 612 | 11.692 | −8.119 | 17.491 | 1.00 | 7.27 | A | N |
| ATOM | 3154 | CA | ALA | A | 612 | 12.922 | −8.703 | 17.973 | 1.00 | 7.36 | A | C |
| ATOM | 3156 | CB | ALA | A | 612 | 13.212 | −9.987 | 17.272 | 1.00 | 7.52 | A | C |
| ATOM | 3160 | C | ALA | A | 612 | 14.090 | −7.718 | 17.877 | 1.00 | 7.40 | A | C |
| ATOM | 3161 | O | ALA | A | 612 | 14.984 | −7.738 | 18.735 | 1.00 | 7.06 | A | O |
| ATOM | 3162 | N | VAL | A | 613 | 14.094 | −6.842 | 16.868 | 1.00 | 7.17 | A | N |
| ATOM | 3164 | CA | VAL | A | 613 | 15.098 | −5.779 | 16.831 | 1.00 | 7.65 | A | C |
| ATOM | 3166 | CB | VAL | A | 613 | 15.096 | −4.939 | 15.516 | 1.00 | 7.18 | A | C |
| ATOM | 3168 | CG1 | VAL | A | 613 | 16.183 | −3.867 | 15.575 | 1.00 | 8.13 | A | C |
| ATOM | 3172 | CG2 | VAL | A | 613 | 15.284 | −5.822 | 14.301 | 1.00 | 8.64 | A | C |
| ATOM | 3176 | C | VAL | A | 613 | 14.874 | −4.843 | 18.018 | 1.00 | 7.44 | A | C |
| ATOM | 3177 | O | VAL | A | 613 | 15.816 | −4.438 | 18.663 | 1.00 | 7.86 | A | O |
| ATOM | 3178 | N | CYS | A | 614 | 13.628 | −4.521 | 18.312 | 1.00 | 7.31 | A | N |
| ATOM | 3180 | CA | CYS | A | 614 | 13.304 | −3.712 | 19.482 | 1.00 | 7.38 | A | C |
| ATOM | 3182 | CB | CYS | A | 614 | 11.808 | −3.409 | 19.500 | 1.00 | 7.62 | A | C |
| ATOM | 3185 | SG | CYS | A | 614 | 11.279 | −2.363 | 20.856 | 1.00 | 12.24 | A | S |
| ATOM | 3186 | C | CYS | A | 614 | 13.803 | −4.393 | 20.791 | 1.00 | 7.51 | A | C |
| ATOM | 3187 | O | CYS | A | 614 | 14.404 | −3.737 | 21.652 | 1.00 | 8.23 | A | O |
| ATOM | 3188 | N | MET | A | 615 | 13.602 | −5.700 | 20.939 | 1.00 | 6.99 | A | N |
| ATOM | 3190 | CA | MET | A | 615 | 14.136 | −6.415 | 22.104 | 1.00 | 8.46 | A | C |
| ATOM | 3192 | CB | MET | A | 615 | 13.727 | −7.893 | 22.087 | 1.00 | 8.91 | A | C |
| ATOM | 3195 | CG | MET | A | 615 | 12.273 | −8.132 | 22.309 | 1.00 | 11.03 | A | C |
| ATOM | 3198 | SD | MET | A | 615 | 12.003 | −9.900 | 22.802 | 1.00 | 14.28 | A | S |
| ATOM | 3199 | CE | MET | A | 615 | 12.231 | −10.684 | 21.273 | 1.00 | 13.21 | A | C |
| ATOM | 3203 | C | MET | A | 615 | 15.657 | −6.314 | 22.182 | 1.00 | 7.71 | A | C |
| ATOM | 3204 | O | MET | A | 615 | 16.223 | −6.111 | 23.249 | 1.00 | 8.82 | A | O |
| ATOM | 3205 | N | TRP | A | 616 | 16.324 | −6.420 | 21.042 | 1.00 | 7.61 | A | N |
| ATOM | 3207 | CA | TRP | A | 616 | 17.773 | −6.243 | 20.982 | 1.00 | 7.16 | A | C |
| ATOM | 3209 | CB | TRP | A | 616 | 18.277 | −6.483 | 19.555 | 1.00 | 7.70 | A | C |
| ATOM | 3212 | CG | TRP | A | 616 | 19.759 | −6.411 | 19.431 | 1.00 | 6.78 | A | C |
| ATOM | 3213 | CD1 | TRP | A | 616 | 20.623 | −7.465 | 19.493 | 1.00 | 7.50 | A | C |
| ATOM | 3215 | NE1 | TRP | A | 616 | 21.916 | −7.021 | 19.337 | 1.00 | 8.34 | A | N |
| ATOM | 3217 | CE2 | TRP | A | 616 | 21.913 | −5.665 | 19.176 | 1.00 | 8.17 | A | C |
| ATOM | 3218 | CD2 | TRP | A | 616 | 20.559 | −5.247 | 19.199 | 1.00 | 5.69 | A | C |
| ATOM | 3219 | CE3 | TRP | A | 616 | 20.285 | −3.876 | 19.074 | 1.00 | 8.29 | A | C |
| ATOM | 3221 | CZ3 | TRP | A | 616 | 21.327 | −3.003 | 18.884 | 1.00 | 8.52 | A | C |
| ATOM | 3223 | CH2 | TRP | A | 616 | 22.663 | −3.460 | 18.833 | 1.00 | 7.88 | A | C |
| ATOM | 3225 | CZ2 | TRP | A | 616 | 22.964 | −4.786 | 18.940 | 1.00 | 8.12 | A | C |
| ATOM | 3227 | C | TRP | A | 616 | 18.171 | −4.841 | 21.476 | 1.00 | 7.53 | A | C |
| ATOM | 3228 | O | TRP | A | 616 | 19.125 | −4.732 | 22.241 | 1.00 | 8.03 | A | O |
| ATOM | 3229 | N | GLU | A | 617 | 17.425 | −3.794 | 21.081 | 1.00 | 7.69 | A | N |
| ATOM | 3231 | CA | GLU | A | 617 | 17.671 | −2.434 | 21.555 | 1.00 | 8.18 | A | C |
| ATOM | 3233 | CB | GLU | A | 617 | 16.718 | −1.416 | 20.918 | 1.00 | 8.57 | A | C |
| ATOM | 3236 | CG | GLU | A | 617 | 16.871 | −1.110 | 19.441 | 1.00 | 10.70 | A | C |
| ATOM | 3239 | CD | GLU | A | 617 | 15.817 | −0.100 | 19.000 | 1.00 | 13.08 | A | C |
| ATOM | 3240 | OE1 | GLU | A | 617 | 16.176 | 0.998 | 18.510 | 1.00 | 13.59 | A | O |
| ATOM | 3241 | OE2 | GLU | A | 617 | 14.620 | −0.372 | 19.205 | 1.00 | 14.34 | A | O |
| ATOM | 3242 | C | GLU | A | 617 | 17.496 | −2.334 | 23.066 | 1.00 | 8.12 | A | C |
| ATOM | 3243 | O | GLU | A | 617 | 18.304 | −1.700 | 23.749 | 1.00 | 8.06 | A | O |
| ATOM | 3244 | N | ILE | A | 618 | 16.453 | −2.970 | 23.593 | 1.00 | 8.10 | A | N |
| ATOM | 3246 | CA | ILE | A | 618 | 16.172 | −2.899 | 25.027 | 1.00 | 8.31 | A | C |
| ATOM | 3248 | CB | ILE | A | 618 | 14.810 | −3.549 | 25.349 | 1.00 | 8.09 | A | C |
| ATOM | 3250 | CG1 | ILE | A | 618 | 13.676 | −2.696 | 24.783 | 1.00 | 7.50 | A | C |
| ATOM | 3253 | CD1 | ILE | A | 618 | 12.296 | −3.333 | 24.895 | 1.00 | 9.45 | A | C |
| ATOM | 3257 | CG2 | ILE | A | 618 | 14.641 | −3.746 | 26.867 | 1.00 | 9.02 | A | C |
| ATOM | 3261 | C | ILE | A | 618 | 17.314 | −3.569 | 25.805 | 1.00 | 8.52 | A | C |
| ATOM | 3262 | O | ILE | A | 618 | 17.884 | −2.983 | 26.731 | 1.00 | 9.07 | A | O |
| ATOM | 3263 | N | LEU | A | 619 | 17.674 | −4.780 | 25.406 | 1.00 | 9.16 | A | N |
| ATOM | 3265 | CA | LEU | A | 619 | 18.752 | −5.517 | 26.063 | 1.00 | 9.73 | A | C |
| ATOM | 3267 | CB | LEU | A | 619 | 18.742 | −6.989 | 25.626 | 1.00 | 10.15 | A | C |
| ATOM | 3270 | CG | LEU | A | 619 | 17.821 | −7.924 | 26.416 | 1.00 | 11.23 | A | C |
| ATOM | 3272 | CD1 | LEU | A | 619 | 18.274 | −8.131 | 27.852 | 1.00 | 12.73 | A | C |
| ATOM | 3276 | CD2 | LEU | A | 619 | 16.384 | −7.464 | 26.361 | 1.00 | 12.78 | A | C |
| ATOM | 3280 | C | LEU | A | 619 | 20.132 | −4.906 | 25.858 | 1.00 | 9.41 | A | C |
| ATOM | 3281 | O | LEU | A | 619 | 21.071 | −5.239 | 26.600 | 1.00 | 11.30 | A | O |
| ATOM | 3282 | N | SER | A | 620 | 20.249 | −3.996 | 24.890 | 1.00 | 9.34 | A | N |
| ATOM | 3284 | CA | SER | A | 620 | 21.466 | −3.226 | 24.656 | 1.00 | 9.57 | A | C |
| ATOM | 3286 | CB | SER | A | 620 | 21.748 | −3.159 | 23.147 | 1.00 | 9.27 | A | C |
| ATOM | 3289 | OG | SER | A | 620 | 21.720 | −4.438 | 22.539 | 1.00 | 9.62 | A | O |
| ATOM | 3291 | C | SER | A | 620 | 21.412 | −1.788 | 25.217 | 1.00 | 10.03 | A | C |
| ATOM | 3292 | O | SER | A | 620 | 22.253 | −0.958 | 24.870 | 1.00 | 10.13 | A | O |
| ATOM | 3293 | N | PHE | A | 621 | 20.438 | −1.495 | 26.072 | 1.00 | 10.21 | A | N |
| ATOM | 3295 | CA | PHE | A | 621 | 20.281 | −0.168 | 26.658 | 1.00 | 10.99 | A | C |
| ATOM | 3297 | CB | PHE | A | 621 | 21.380 | 0.092 | 27.686 | 1.00 | 11.57 | A | C |
| ATOM | 3300 | CG | PHE | A | 621 | 21.417 | −0.916 | 28.786 | 1.00 | 11.76 | A | C |
| ATOM | 3301 | CD1 | PHE | A | 621 | 20.585 | −0.777 | 29.903 | 1.00 | 14.73 | A | C |
| ATOM | 3303 | CE1 | PHE | A | 621 | 20.616 | −1.720 | 30.939 | 1.00 | 13.38 | A | C |
| ATOM | 3305 | CZ | PHE | A | 621 | 21.455 | −2.806 | 30.845 | 1.00 | 15.10 | A | C |
| ATOM | 3307 | CE2 | PHE | A | 621 | 22.282 | −2.966 | 29.728 | 1.00 | 15.45 | A | C |
| ATOM | 3309 | CD2 | PHE | A | 621 | 22.257 | −2.020 | 28.708 | 1.00 | 14.11 | A | C |
| ATOM | 3311 | C | PHE | A | 621 | 20.204 | 0.974 | 25.633 | 1.00 | 11.51 | A | C |
| ATOM | 3312 | O | PHE | A | 621 | 20.746 | 2.054 | 25.840 | 1.00 | 11.76 | A | O |
| ATOM | 3313 | N | GLY | A | 622 | 19.533 | 0.715 | 24.513 | 1.00 | 12.15 | A | N |
| ATOM | 3315 | CA | GLY | A | 622 | 19.195 | 1.755 | 23.561 | 1.00 | 12.52 | A | C |
| ATOM | 3318 | C | GLY | A | 622 | 20.125 | 1.958 | 22.389 | 1.00 | 13.31 | A | C |
| ATOM | 3319 | O | GLY | A | 622 | 19.957 | 2.916 | 21.631 | 1.00 | 13.27 | A | O |
| ATOM | 3320 | N | LYS | A | 623 | 21.117 | 1.086 | 22.224 | 1.00 | 13.94 | A | N |
| ATOM | 3322 | CA | LYS | A | 623 | 22.003 | 1.199 | 21.072 | 1.00 | 14.20 | A | C |
| ATOM | 3324 | CB | LYS | A | 623 | 23.120 | 0.167 | 21.156 | 1.00 | 15.30 | A | C |
| ATOM | 3327 | CG | LYS | A | 623 | 24.036 | 0.374 | 22.340 | 1.00 | 18.22 | A | C |
| ATOM | 3330 | CD | LYS | A | 623 | 25.359 | −0.349 | 22.137 | 1.00 | 23.13 | A | C |
| ATOM | 3333 | CE | LYS | A | 623 | 25.198 | −1.851 | 22.058 | 1.00 | 23.04 | A | C |
| ATOM | 3336 | NZ | LYS | A | 623 | 26.022 | −2.515 | 23.072 | 1.00 | 22.31 | A | N |
| ATOM | 3340 | C | LYS | A | 623 | 21.223 | 1.016 | 19.765 | 1.00 | 13.35 | A | C |
| ATOM | 3341 | O | LYS | A | 623 | 20.175 | 0.354 | 19.732 | 1.00 | 12.25 | A | O |
| ATOM | 3342 | N | GLN | A | 624 | 21.720 | 1.631 | 18.694 | 1.00 | 12.33 | A | N |
| ATOM | 3344 | CA | GLN | A | 624 | 21.074 | 1.532 | 17.390 | 1.00 | 11.79 | A | C |
| ATOM | 3346 | CB | GLN | A | 624 | 21.471 | 2.693 | 16.471 | 1.00 | 12.79 | A | C |
| ATOM | 3349 | CG | GLN | A | 624 | 20.803 | 2.653 | 15.092 | 1.00 | 16.84 | A | C |
| ATOM | 3352 | CD | GLN | A | 624 | 21.480 | 3.512 | 14.033 | 1.00 | 20.72 | A | C |
| ATOM | 3353 | OE1 | GLN | A | 624 | 22.510 | 3.139 | 13.484 | 1.00 | 22.00 | A | O |
| ATOM | 3354 | NE2 | GLN | A | 624 | 20.863 | 4.642 | 13.706 | 1.00 | 25.56 | A | N |
| ATOM | 3357 | C | GLN | A | 624 | 21.484 | 0.218 | 16.750 | 1.00 | 10.43 | A | C |
| ATOM | 3358 | O | GLN | A | 624 | 22.674 | −0.085 | 16.700 | 1.00 | 9.05 | A | O |
| ATOM | 3359 | N | PRO | A | 625 | 20.527 | −0.543 | 16.226 | 1.00 | 9.21 | A | N |
| ATOM | 3360 | CA | PRO | A | 625 | 20.857 | −1.773 | 15.510 | 1.00 | 8.85 | A | C |
| ATOM | 3362 | CB | PRO | A | 625 | 19.489 | −2.336 | 15.099 | 1.00 | 8.81 | A | C |
| ATOM | 3365 | CG | PRO | A | 625 | 18.589 | −1.182 | 15.135 | 1.00 | 8.12 | A | C |
| ATOM | 3368 | CD | PRO | A | 625 | 19.077 | −0.284 | 16.226 | 1.00 | 8.87 | A | C |
| ATOM | 3371 | C | PRO | A | 625 | 21.690 | −1.483 | 14.260 | 1.00 | 9.18 | A | C |
| ATOM | 3372 | O | PRO | A | 625 | 21.427 | −0.517 | 13.529 | 1.00 | 8.70 | A | O |
| ATOM | 3373 | N | PHE | A | 626 | 22.699 | −2.312 | 14.017 | 1.00 | 8.97 | A | N |
| ATOM | 3375 | CA | PHE | A | 626 | 23.530 | −2.167 | 12.825 | 1.00 | 8.80 | A | C |
| ATOM | 3241 | OE2 | GLU | A | 617 | 14.620 | −0.372 | 19.205 | 1.00 | 14.34 | A | O |
| ATOM | 3242 | C | GLU | A | 617 | 17.496 | −2.334 | 23.066 | 1.00 | 8.12 | A | C |
| ATOM | 3243 | O | GLU | A | 617 | 18.304 | −1.700 | 23.749 | 1.00 | 8.06 | A | O |
| ATOM | 3244 | N | ILE | A | 618 | 16.453 | −2.970 | 23.593 | 1.00 | 8.10 | A | N |
| ATOM | 3246 | CA | ILE | A | 618 | 16.172 | −2.899 | 25.027 | 1.00 | 8.31 | A | C |
| ATOM | 3248 | CB | ILE | A | 618 | 14.810 | −3.549 | 25.349 | 1.00 | 8.09 | A | C |
| ATOM | 3250 | CG1 | ILE | A | 618 | 13.676 | −2.696 | 24.783 | 1.00 | 7.50 | A | C |
| ATOM | 3253 | CD1 | ILE | A | 618 | 12.296 | −3.333 | 24.895 | 1.00 | 9.45 | A | C |
| ATOM | 3257 | CG2 | ILE | A | 618 | 14.641 | −3.746 | 26.867 | 1.00 | 9.02 | A | C |
| ATOM | 3261 | C | ILE | A | 618 | 17.314 | −3.569 | 25.805 | 1.00 | 8.52 | A | C |
| ATOM | 3262 | O | ILE | A | 618 | 17.884 | −2.983 | 26.731 | 1.00 | 9.07 | A | O |
| ATOM | 3263 | N | LEU | A | 619 | 17.674 | −4.780 | 25.406 | 1.00 | 9.16 | A | N |
| ATOM | 3265 | CA | LEU | A | 619 | 18.752 | −5.517 | 26.063 | 1.00 | 9.73 | A | C |
| ATOM | 3267 | CB | LEU | A | 619 | 18.742 | −6.989 | 25.626 | 1.00 | 10.15 | A | C |
| ATOM | 3270 | CG | LEU | A | 619 | 17.821 | −7.924 | 26.416 | 1.00 | 11.23 | A | C |
| ATOM | 3272 | CD1 | LEU | A | 619 | 18.274 | −8.131 | 27.852 | 1.00 | 12.73 | A | C |
| ATOM | 3276 | CD2 | LEU | A | 619 | 16.384 | −7.464 | 26.361 | 1.00 | 12.78 | A | C |
| ATOM | 3280 | C | LEU | A | 619 | 20.132 | −4.906 | 25.858 | 1.00 | 9.41 | A | C |
| ATOM | 3281 | O | LEU | A | 619 | 21.071 | −5.239 | 26.600 | 1.00 | 11.30 | A | O |
| ATOM | 3282 | N | SER | A | 620 | 20.249 | −3.996 | 24.890 | 1.00 | 9.34 | A | N |
| ATOM | 3284 | CA | SER | A | 620 | 21.466 | −3.226 | 24.656 | 1.00 | 9.57 | A | C |
| ATOM | 3286 | CB | SER | A | 620 | 21.748 | −3.159 | 23.147 | 1.00 | 9.27 | A | C |
| ATOM | 3289 | OG | SER | A | 620 | 21.720 | −4.438 | 22.539 | 1.00 | 9.62 | A | O |
| ATOM | 3291 | C | SER | A | 620 | 21.412 | −1.788 | 25.217 | 1.00 | 10.03 | A | C |
| ATOM | 3292 | O | SER | A | 620 | 22.253 | −0.958 | 24.870 | 1.00 | 10.13 | A | O |
| ATOM | 3293 | N | PHE | A | 621 | 20.438 | −1.495 | 26.072 | 1.00 | 10.21 | A | N |
| ATOM | 3295 | CA | PHE | A | 621 | 20.281 | −0.168 | 26.658 | 1.00 | 10.99 | A | C |
| ATOM | 3297 | CB | PHE | A | 621 | 21.380 | 0.092 | 27.686 | 1.00 | 11.57 | A | C |
| ATOM | 3300 | CG | PHE | A | 621 | 21.417 | −0.916 | 28.786 | 1.00 | 11.76 | A | C |
| ATOM | 3301 | CD1 | PHE | A | 621 | 20.585 | −0.777 | 29.903 | 1.00 | 14.73 | A | C |
| ATOM | 3303 | CE1 | PHE | A | 621 | 20.616 | −1.720 | 30.939 | 1.00 | 13.38 | A | C |
| ATOM | 3305 | CZ | PHE | A | 621 | 21.455 | −2.806 | 30.845 | 1.00 | 15.10 | A | C |
| ATOM | 3307 | CE2 | PHE | A | 621 | 22.282 | −2.966 | 29.728 | 1.00 | 15.45 | A | C |
| ATOM | 3309 | CD2 | PHE | A | 621 | 22.257 | −2.020 | 28.708 | 1.00 | 14.11 | A | C |
| ATOM | 3311 | C | PHE | A | 621 | 20.204 | 0.974 | 25.633 | 1.00 | 11.51 | A | C |
| ATOM | 3312 | O | PHE | A | 621 | 20.746 | 2.054 | 25.840 | 1.00 | 11.76 | A | O |
| ATOM | 3313 | N | GLY | A | 622 | 19.533 | 0.715 | 24.513 | 1.00 | 12.15 | A | N |
| ATOM | 3315 | CA | GLY | A | 622 | 19.195 | 1.755 | 23.561 | 1.00 | 12.52 | A | C |
| ATOM | 3318 | C | GLY | A | 622 | 20.125 | 1.958 | 22.389 | 1.00 | 13.31 | A | C |
| ATOM | 3319 | O | GLY | A | 622 | 19.957 | 2.916 | 21.631 | 1.00 | 13.27 | A | O |
| ATOM | 3320 | N | LYS | A | 623 | 21.117 | 1.086 | 22.224 | 1.00 | 13.94 | A | N |
| ATOM | 3322 | CA | LYS | A | 623 | 22.003 | 1.199 | 21.072 | 1.00 | 14.20 | A | C |
| ATOM | 3324 | CB | LYS | A | 623 | 23.120 | 0.167 | 21.156 | 1.00 | 15.30 | A | C |
| ATOM | 3327 | CG | LYS | A | 623 | 24.036 | 0.374 | 22.340 | 1.00 | 18.22 | A | C |
| ATOM | 3330 | CD | LYS | A | 623 | 25.359 | −0.349 | 22.137 | 1.00 | 23.13 | A | C |
| ATOM | 3333 | CE | LYS | A | 623 | 25.198 | −1.851 | 22.058 | 1.00 | 23.04 | A | C |
| ATOM | 3336 | NZ | LYS | A | 623 | 26.022 | −2.515 | 23.072 | 1.00 | 22.31 | A | N |
| ATOM | 3340 | C | LYS | A | 623 | 21.223 | 1.016 | 19.765 | 1.00 | 13.35 | A | C |
| ATOM | 3341 | O | LYS | A | 623 | 20.175 | 0.354 | 19.732 | 1.00 | 12.25 | A | O |
| ATOM | 3342 | N | GLN | A | 624 | 21.720 | 1.631 | 18.694 | 1.00 | 12.33 | A | N |
| ATOM | 3344 | CA | GLN | A | 624 | 21.074 | 1.532 | 17.390 | 1.00 | 11.79 | A | C |
| ATOM | 3346 | CB | GLN | A | 624 | 21.471 | 2.693 | 16.471 | 1.00 | 12.79 | A | C |
| ATOM | 3349 | CG | GLN | A | 624 | 20.803 | 2.653 | 15.092 | 1.00 | 16.84 | A | C |
| ATOM | 3352 | CD | GLN | A | 624 | 21.480 | 3.512 | 14.033 | 1.00 | 20.72 | A | C |
| ATOM | 3353 | OE1 | GLN | A | 624 | 22.510 | 3.139 | 13.484 | 1.00 | 22.00 | A | O |
| ATOM | 3354 | NE2 | GLN | A | 624 | 20.863 | 4.642 | 13.706 | 1.00 | 25.56 | A | N |
| ATOM | 3357 | C | GLN | A | 624 | 21.484 | 0.218 | 16.750 | 1.00 | 10.43 | A | C |
| ATOM | 3358 | O | GLN | A | 624 | 22.674 | −0.085 | 16.700 | 1.00 | 9.05 | A | O |
| ATOM | 3359 | N | PRO | A | 625 | 20.527 | −0.543 | 16.226 | 1.00 | 9.21 | A | N |
| ATOM | 3360 | CA | PRO | A | 625 | 20.857 | −1.773 | 15.510 | 1.00 | 8.85 | A | C |
| ATOM | 3362 | CB | PRO | A | 625 | 19.489 | −2.336 | 15.099 | 1.00 | 8.81 | A | C |
| ATOM | 3365 | CG | PRO | A | 625 | 18.589 | −1.182 | 15.135 | 1.00 | 8.12 | A | C |
| ATOM | 3368 | CD | PRO | A | 625 | 19.077 | −0.284 | 16.226 | 1.00 | 8.87 | A | C |
| ATOM | 3371 | C | PRO | A | 625 | 21.690 | −1.483 | 14.260 | 1.00 | 9.18 | A | C |
| ATOM | 3372 | O | PRO | A | 625 | 21.427 | −0.517 | 13.529 | 1.00 | 8.70 | A | O |
| ATOM | 3373 | N | PHE | A | 626 | 22.699 | −2.312 | 14.017 | 1.00 | 8.97 | A | N |
| ATOM | 3375 | CA | PHE | A | 626 | 23.530 | −2.167 | 12.825 | 1.00 | 8.80 | A | C |
| ATOM | 3377 | CB | PHE | A | 626 | 22.755 | −2.516 | 11.552 | 1.00 | 9.35 | A | C |
| ATOM | 3380 | CG | PHE | A | 626 | 22.317 | −3.959 | 11.476 | 1.00 | 7.57 | A | C |
| ATOM | 3381 | CD1 | PHE | A | 626 | 23.263 | −4.982 | 11.428 | 1.00 | 8.74 | A | C |
| ATOM | 3383 | CE1 | PHE | A | 626 | 22.880 | −6.293 | 11.333 | 1.00 | 7.90 | A | C |
| ATOM | 3385 | CZ | PHE | A | 626 | 21.544 | −6.621 | 11.266 | 1.00 | 9.02 | A | C |
| ATOM | 3387 | CE2 | PHE | A | 626 | 20.584 | −5.630 | 11.313 | 1.00 | 7.58 | A | C |
| ATOM | 3389 | CD2 | PHE | A | 626 | 20.967 | −4.303 | 11.402 | 1.00 | 7.59 | A | C |
| ATOM | 3391 | C | PHE | A | 626 | 24.153 | −0.762 | 12.737 | 1.00 | 9.81 | A | C |
| ATOM | 3392 | O | PHE | A | 626 | 24.281 | −0.193 | 11.669 | 1.00 | 9.56 | A | O |
| ATOM | 3393 | N | PHE | A | 627 | 24.554 | −0.231 | 13.879 | 1.00 | 9.82 | A | N |
| ATOM | 3395 | CA | PHE | A | 627 | 25.190 | 1.088 | 13.917 | 1.00 | 11.48 | A | C |
| ATOM | 3397 | CB | PHE | A | 627 | 25.448 | 1.543 | 15.365 | 1.00 | 11.15 | A | C |
| ATOM | 3400 | CG | PHE | A | 627 | 26.395 | 0.662 | 16.150 | 1.00 | 11.35 | A | C |
| ATOM | 3401 | CD1 | PHE | A | 627 | 27.774 | 0.810 | 16.042 | 1.00 | 9.33 | A | C |
| ATOM | 3403 | CE1 | PHE | A | 627 | 28.637 | 0.017 | 16.772 | 1.00 | 10.69 | A | C |
| ATOM | 3405 | CZ | PHE | A | 627 | 28.131 | −0.911 | 17.652 | 1.00 | 12.10 | A | C |
| ATOM | 3407 | CE2 | PHE | A | 627 | 26.746 | −1.042 | 17.787 | 1.00 | 10.84 | A | C |
| ATOM | 3409 | CD2 | PHE | A | 627 | 25.905 | −0.256 | 17.051 | 1.00 | 9.05 | A | C |
| ATOM | 3411 | C | PHE | A | 627 | 26.475 | 1.142 | 13.092 | 1.00 | 12.51 | A | C |
| ATOM | 3412 | O | PHE | A | 627 | 26.881 | 2.214 | 12.636 | 1.00 | 14.55 | A | O |
| ATOM | 3413 | N | TRP | A | 628 | 27.076 | −0.021 | 12.879 | 1.00 | 12.47 | A | N |
| ATOM | 3415 | CA | TRP | A | 628 | 28.355 | −0.158 | 12.189 | 1.00 | 13.92 | A | C |
| ATOM | 3417 | CB | TRP | A | 628 | 29.085 | −1.409 | 12.710 | 1.00 | 13.56 | A | C |
| ATOM | 3420 | CG | TRP | A | 628 | 28.298 | −2.730 | 12.615 | 1.00 | 13.24 | A | C |
| ATOM | 3421 | CD1 | TRP | A | 628 | 28.356 | −3.647 | 11.600 | 1.00 | 13.12 | A | C |
| ATOM | 3423 | NE1 | TRP | A | 628 | 27.528 | −4.710 | 11.871 | 1.00 | 14.92 | A | N |
| ATOM | 3425 | CE2 | TRP | A | 628 | 26.894 | −4.493 | 13.060 | 1.00 | 13.87 | A | C |
| ATOM | 3426 | CD2 | TRP | A | 628 | 27.366 | −3.256 | 13.566 | 1.00 | 12.08 | A | C |
| ATOM | 3427 | CE3 | TRP | A | 628 | 26.861 | −2.807 | 14.784 | 1.00 | 13.25 | A | C |
| ATOM | 3429 | CZ3 | TRP | A | 628 | 25.928 | −3.597 | 15.468 | 1.00 | 13.18 | A | C |
| ATOM | 3431 | CH2 | TRP | A | 628 | 25.490 | −4.812 | 14.935 | 1.00 | 12.66 | A | C |
| ATOM | 3433 | CZ2 | TRP | A | 628 | 25.966 | −5.276 | 13.739 | 1.00 | 12.17 | A | C |
| ATOM | 3435 | C | TRP | A | 628 | 28.204 | −0.203 | 10.658 | 1.00 | 14.82 | A | C |
| ATOM | 3436 | O | TRP | A | 628 | 29.199 | −0.297 | 9.938 | 1.00 | 16.19 | A | O |
| ATOM | 3437 | N | LEU | A | 629 | 26.963 | −0.177 | 10.171 | 1.00 | 14.95 | A | N |
| ATOM | 3439 | CA | LEU | A | 629 | 26.653 | −0.266 | 8.744 | 1.00 | 15.95 | A | C |
| ATOM | 3441 | CB | LEU | A | 629 | 25.711 | −1.447 | 8.475 | 1.00 | 15.63 | A | C |
| ATOM | 3444 | CG | LEU | A | 629 | 26.128 | −2.866 | 8.839 | 1.00 | 15.85 | A | C |
| ATOM | 3446 | CD1 | LEU | A | 629 | 25.034 | −3.791 | 8.377 | 1.00 | 17.66 | A | C |
| ATOM | 3450 | CD2 | LEU | A | 629 | 27.437 | −3.241 | 8.191 | 1.00 | 17.21 | A | C |
| ATOM | 3454 | C | LEU | A | 629 | 25.957 | 0.991 | 8.249 | 1.00 | 16.16 | A | C |
| ATOM | 3455 | O | LEU | A | 629 | 25.422 | 1.767 | 9.042 | 1.00 | 17.19 | A | O |
| ATOM | 3456 | N | GLU | A | 630 | 26.012 | 1.200 | 6.933 | 1.00 | 17.08 | A | N |
| ATOM | 3458 | CA | GLU | A | 630 | 25.159 | 2.176 | 6.244 | 1.00 | 17.66 | A | C |
| ATOM | 3460 | CB | GLU | A | 630 | 25.859 | 2.759 | 4.990 | 1.00 | 18.54 | A | C |
| ATOM | 3463 | CG | GLU | A | 630 | 27.107 | 3.592 | 5.285 | 1.00 | 22.16 | A | C |
| ATOM | 3466 | CD | GLU | A | 630 | 27.834 | 4.104 | 4.035 | 1.00 | 26.66 | A | C |
| ATOM | 3467 | OE1 | GLU | A | 630 | 28.025 | 3.347 | 3.054 | 1.00 | 30.10 | A | O |
| ATOM | 3468 | OE2 | GLU | A | 630 | 28.254 | 5.276 | 4.041 | 1.00 | 31.44 | A | O |
| ATOM | 3469 | C | GLU | A | 630 | 23.877 | 1.444 | 5.840 | 1.00 | 16.80 | A | C |
| ATOM | 3470 | O | GLU | A | 630 | 23.899 | 0.232 | 5.644 | 1.00 | 16.52 | A | O |
| ATOM | 3471 | N | ASN | A | 631 | 22.777 | 2.177 | 5.693 | 1.00 | 16.64 | A | N |
| ATOM | 3473 | CA | ASN | A | 631 | 21.494 | 1.588 | 5.288 | 1.00 | 16.86 | A | C |
| ATOM | 3475 | CB | ASN | A | 631 | 20.444 | 2.692 | 5.027 | 1.00 | 16.92 | A | C |
| ATOM | 3478 | CG | ASN | A | 631 | 19.758 | 3.184 | 6.315 | 1.00 | 18.23 | A | C |
| ATOM | 3479 | OD1 | ASN | A | 631 | 19.981 | 2.645 | 7.395 | 1.00 | 19.71 | A | O |
| ATOM | 3480 | ND2 | ASN | A | 631 | 18.935 | 4.230 | 6.196 | 1.00 | 18.99 | A | N |
| ATOM | 3483 | C | ASN | A | 631 | 21.626 | 0.674 | 4.067 | 1.00 | 16.54 | A | C |
| ATOM | 3484 | O | ASN | A | 631 | 21.011 | −0.395 | 4.008 | 1.00 | 16.91 | A | O |
| ATOM | 3485 | N | LYS | A | 632 | 22.435 | 1.094 | 3.097 | 1.00 | 17.10 | A | N |
| ATOM | 3487 | CA | LYS | A | 632 | 22.531 | 0.396 | 1.817 | 1.00 | 17.56 | A | C |
| ATOM | 3489 | CB | LYS | A | 632 | 23.263 | 1.260 | 0.770 | 1.00 | 17.78 | A | C |
| ATOM | 3492 | CG | LYS | A | 632 | 24.756 | 1.461 | 0.990 | 1.00 | 19.40 | A | C |
| ATOM | 3495 | CD | LYS | A | 632 | 25.297 | 2.438 | −0.081 | 1.00 | 23.92 | A | C |
| ATOM | 3498 | CE | LYS | A | 632 | 26.787 | 2.688 | 0.044 | 1.00 | 24.81 | A | C |
| ATOM | 3501 | NZ | LYS | A | 632 | 27.592 | 1.430 | 0.014 | 1.00 | 27.18 | A | N |
| ATOM | 3505 | C | LYS | A | 632 | 23.194 | −0.965 | 1.940 | 1.00 | 17.38 | A | C |
| ATOM | 3506 | O | LYS | A | 632 | 23.086 | −1.798 | 1.040 | 1.00 | 17.61 | A | O |
| ATOM | 3507 | N | ASP | A | 633 | 23.863 | −1.195 | 3.064 | 1.00 | 17.62 | A | N |
| ATOM | 3509 | CA | ASP | A | 633 | 24.618 | −2.423 | 3.287 | 1.00 | 17.79 | A | C |
| ATOM | 3511 | CB | ASP | A | 633 | 25.892 | −2.111 | 4.070 | 1.00 | 18.50 | A | C |
| ATOM | 3514 | CG | ASP | A | 633 | 26.869 | −1.242 | 3.289 | 1.00 | 22.13 | A | C |
| ATOM | 3515 | OD1 | ASP | A | 633 | 27.019 | −1.425 | 2.062 | 1.00 | 25.82 | A | O |
| ATOM | 3516 | OD2 | ASP | A | 633 | 27.534 | −0.344 | 3.843 | 1.00 | 29.08 | A | O |
| ATOM | 3517 | C | ASP | A | 633 | 23.830 | −3.477 | 4.054 | 1.00 | 16.35 | A | C |
| ATOM | 3518 | O | ASP | A | 633 | 24.244 | −4.631 | 4.106 | 1.00 | 16.00 | A | O |
| ATOM | 3519 | N | VAL | A | 634 | 22.698 | −3.086 | 4.639 | 1.00 | 14.77 | A | N |
| ATOM | 3521 | CA | VAL | A | 634 | 21.941 | −3.962 | 5.535 | 1.00 | 14.11 | A | C |
| ATOM | 3523 | CB | VAL | A | 634 | 20.767 | −3.188 | 6.199 | 1.00 | 14.20 | A | C |
| ATOM | 3525 | CG1 | VAL | A | 634 | 19.834 | −4.110 | 6.957 | 1.00 | 14.96 | A | C |
| ATOM | 3529 | CG2 | VAL | A | 634 | 21.298 | −2.120 | 7.131 | 1.00 | 15.06 | A | C |
| ATOM | 3533 | C | VAL | A | 634 | 21.448 | −5.224 | 4.823 | 1.00 | 13.43 | A | C |
| ATOM | 3534 | O | VAL | A | 634 | 21.685 | −6.339 | 5.289 | 1.00 | 12.23 | A | O |
| ATOM | 3535 | N | ILE | A | 635 | 20.792 | −5.063 | 3.676 | 1.00 | 13.66 | A | N |
| ATOM | 3537 | CA | ILE | A | 635 | 20.175 | −6.214 | 3.010 | 1.00 | 13.87 | A | C |
| ATOM | 3539 | CB | ILE | A | 635 | 19.308 | −5.792 | 1.786 | 1.00 | 14.01 | A | C |
| ATOM | 3541 | CG1 | ILE | A | 635 | 18.407 | −6.942 | 1.344 | 1.00 | 13.90 | A | C |
| ATOM | 3544 | CD1 | ILE | A | 635 | 17.406 | −7.408 | 2.394 | 1.00 | 14.48 | A | C |
| ATOM | 3548 | CG2 | ILE | A | 635 | 20.169 | −5.256 | 0.628 | 1.00 | 14.55 | A | C |
| ATOM | 3552 | C | ILE | A | 635 | 21.205 | −7.287 | 2.666 | 1.00 | 14.30 | A | C |
| ATOM | 3553 | O | ILE | A | 635 | 20.961 | −8.467 | 2.912 | 1.00 | 13.32 | A | O |
| ATOM | 3554 | N | GLY | A | 636 | 22.369 | −6.865 | 2.167 | 1.00 | 14.75 | A | N |
| ATOM | 3556 | CA | GLY | A | 636 | 23.483 | −7.771 | 1.868 | 1.00 | 14.87 | A | C |
| ATOM | 3559 | C | GLY | A | 636 | 23.898 | −8.630 | 3.048 | 1.00 | 14.75 | A | C |
| ATOM | 3560 | O | GLY | A | 636 | 24.068 | −9.846 | 2.937 | 1.00 | 14.77 | A | O |
| ATOM | 3561 | N | VAL | A | 637 | 24.046 | −7.989 | 4.195 | 1.00 | 14.62 | A | N |
| ATOM | 3563 | CA | VAL | A | 637 | 24.396 | −8.669 | 5.431 | 1.00 | 14.71 | A | C |
| ATOM | 3565 | CB | VAL | A | 637 | 24.620 | −7.610 | 6.548 | 1.00 | 15.24 | A | C |
| ATOM | 3567 | CG1 | VAL | A | 637 | 24.587 | −8.215 | 7.928 | 1.00 | 15.93 | A | C |
| ATOM | 3571 | CG2 | VAL | A | 637 | 25.932 | −6.869 | 6.295 | 1.00 | 16.95 | A | C |
| ATOM | 3575 | C | VAL | A | 637 | 23.336 | −9.700 | 5.841 | 1.00 | 14.05 | A | C |
| ATOM | 3576 | O | VAL | A | 637 | 23.644 | −10.829 | 6.188 | 1.00 | 13.96 | A | O |
| ATOM | 3577 | N | LEU | A | 638 | 22.074 | −9.322 | 5.779 | 1.00 | 13.42 | A | N |
| ATOM | 3579 | CA | LEU | A | 638 | 20.995 | −10.231 | 6.146 | 1.00 | 13.52 | A | C |
| ATOM | 3581 | CB | LEU | A | 638 | 19.669 | −9.472 | 6.179 | 1.00 | 13.37 | A | C |
| ATOM | 3584 | CG | LEU | A | 638 | 19.587 | −8.329 | 7.203 | 1.00 | 12.38 | A | C |
| ATOM | 3586 | CD1 | LEU | A | 638 | 18.240 | −7.644 | 7.078 | 1.00 | 12.99 | A | C |
| ATOM | 3590 | CD2 | LEU | A | 638 | 19.783 | −8.855 | 8.587 | 1.00 | 13.05 | A | C |
| ATOM | 3594 | C | LEU | A | 638 | 20.902 | −11.427 | 5.188 | 1.00 | 14.41 | A | C |
| ATOM | 3595 | O | LEU | A | 638 | 20.640 | −12.547 | 5.617 | 1.00 | 13.64 | A | O |
| ATOM | 3596 | N | GLU | A | 639 | 21.119 | −11.170 | 3.897 | 1.00 | 15.99 | A | N |
| ATOM | 3598 | CA | GLU | A | 639 | 21.032 | −12.204 | 2.860 | 1.00 | 17.38 | A | C |
| ATOM | 3600 | CB | GLU | A | 639 | 20.974 | −11.585 | 1.446 | 1.00 | 17.84 | A | C |
| ATOM | 3603 | CG | GLU | A | 639 | 19.567 | −11.053 | 1.155 | 1.00 | 19.95 | A | C |
| ATOM | 3606 | CD | GLU | A | 639 | 19.349 | −10.385 | −0.196 | 1.00 | 24.27 | A | C |
| ATOM | 3607 | OE1 | GLU | A | 639 | 20.314 | −9.998 | −0.890 | 1.00 | 26.43 | A | O |
| ATOM | 3608 | OE2 | GLU | A | 639 | 18.156 | −10.234 | −0.555 | 1.00 | 28.41 | A | O |
| ATOM | 3609 | C | GLU | A | 639 | 22.171 | −13.190 | 3.015 | 1.00 | 18.02 | A | C |
| ATOM | 3610 | O | GLU | A | 639 | 21.998 | −14.362 | 2.755 | 1.00 | 18.32 | A | O |
| ATOM | 3611 | N | LYS | A | 640 | 23.319 | −12.733 | 3.503 | 1.00 | 18.92 | A | N |
| ATOM | 3613 | CA | LYS | A | 640 | 24.431 | −13.645 | 3.751 | 1.00 | 19.98 | A | C |
| ATOM | 3615 | CB | LYS | A | 640 | 25.777 | −12.903 | 3.693 | 1.00 | 20.94 | A | C |
| ATOM | 3618 | CG | LYS | A | 640 | 26.233 | −12.229 | 4.965 | 1.00 | 24.88 | A | C |
| ATOM | 3621 | CD | LYS | A | 640 | 27.432 | −11.309 | 4.704 | 1.00 | 28.51 | A | C |
| ATOM | 3624 | CE | LYS | A | 640 | 28.699 | −12.102 | 4.445 | 1.00 | 30.97 | A | C |
| ATOM | 3627 | NZ | LYS | A | 640 | 29.922 | −11.339 | 4.842 | 1.00 | 32.88 | A | N |
| ATOM | 3631 | C | LYS | A | 640 | 24.244 | −14.456 | 5.038 | 1.00 | 19.37 | A | C |
| ATOM | 3632 | O | LYS | A | 640 | 25.047 | −15.343 | 5.324 | 1.00 | 20.32 | A | O |
| ATOM | 3633 | N | GLY | A | 641 | 23.172 | −14.188 | 5.795 | 1.00 | 18.02 | A | N |
| ATOM | 3635 | CA | GLY | A | 641 | 22.864 | −14.947 | 7.006 | 1.00 | 17.51 | A | C |
| ATOM | 3638 | C | GLY | A | 641 | 23.309 | −14.328 | 8.324 | 1.00 | 16.94 | A | C |
| ATOM | 3639 | O | GLY | A | 641 | 23.063 | −14.876 | 9.398 | 1.00 | 17.26 | A | O |
| ATOM | 3640 | N | ASP | A | 642 | 23.978 | −13.186 | 8.260 | 1.00 | 15.83 | A | N |
| ATOM | 3642 | CA | ASP | A | 642 | 24.426 | −12.516 | 9.476 | 1.00 | 15.01 | A | C |
| ATOM | 3644 | CB | ASP | A | 642 | 25.482 | −11.476 | 9.155 | 1.00 | 15.19 | A | C |
| ATOM | 3647 | CG | ASP | A | 642 | 26.786 | −12.079 | 8.651 | 1.00 | 18.80 | A | C |
| ATOM | 3648 | OD1 | ASP | A | 642 | 27.065 | −13.269 | 8.921 | 1.00 | 19.61 | A | O |
| ATOM | 3649 | OD2 | ASP | A | 642 | 27.586 | −11.400 | 7.975 | 1.00 | 23.88 | A | O |
| ATOM | 3650 | C | ASP | A | 642 | 23.249 | −11.815 | 10.152 | 1.00 | 13.57 | A | C |
| ATOM | 3651 | O | ASP | A | 642 | 22.307 | −11.354 | 9.490 | 1.00 | 12.33 | A | O |
| ATOM | 3652 | N | ARG | A | 643 | 23.362 | −11.697 | 11.464 | 1.00 | 12.50 | A | N |
| ATOM | 3654 | CA | ARG | A | 643 | 22.331 | −11.111 | 12.317 | 1.00 | 12.39 | A | C |
| ATOM | 3656 | CB | ARG | A | 643 | 21.489 | −12.225 | 12.953 | 1.00 | 12.39 | A | C |
| ATOM | 3659 | CG | ARG | A | 643 | 20.694 | −13.090 | 11.983 | 1.00 | 12.03 | A | C |
| ATOM | 3662 | CD | ARG | A | 643 | 19.623 | −12.348 | 11.205 | 1.00 | 11.99 | A | C |
| ATOM | 3665 | NE | ARG | A | 643 | 18.815 | −13.225 | 10.362 | 1.00 | 13.20 | A | N |
| ATOM | 3667 | CZ | ARG | A | 643 | 19.009 | −13.447 | 9.066 | 1.00 | 12.12 | A | C |
| ATOM | 3668 | NH1 | ARG | A | 643 | 20.017 | −12.891 | 8.422 | 1.00 | 12.02 | A | N |
| ATOM | 3671 | NH2 | ARG | A | 643 | 18.192 | −14.246 | 8.407 | 1.00 | 13.42 | A | N |
| ATOM | 3674 | C | ARG | A | 643 | 22.971 | −10.248 | 13.403 | 1.00 | 11.47 | A | C |
| ATOM | 3675 | O | ARG | A | 643 | 24.193 | −10.287 | 13.620 | 1.00 | 12.13 | A | O |
| ATOM | 3676 | N | LEU | A | 644 | 22.153 | −9.467 | 14.099 | 1.00 | 10.94 | A | N |
| ATOM | 3678 | CA | LEU | A | 644 | 22.594 | −8.737 | 15.290 | 1.00 | 10.40 | A | C |
| ATOM | 3680 | CB | LEU | A | 644 | 21.419 | −7.958 | 15.904 | 1.00 | 9.69 | A | C |
| ATOM | 3683 | CG | LEU | A | 644 | 20.852 | −6.832 | 15.018 | 1.00 | 11.08 | A | C |
| ATOM | 3685 | CD1 | LEU | A | 644 | 19.472 | −6.365 | 15.508 | 1.00 | 9.53 | A | C |
| ATOM | 3689 | CD2 | LEU | A | 644 | 21.813 | −5.666 | 14.973 | 1.00 | 10.40 | A | C |
| ATOM | 3693 | C | LEU | A | 644 | 23.159 | −9.720 | 16.313 | 1.00 | 10.18 | A | C |
| ATOM | 3694 | O | LEU | A | 644 | 22.586 | −10.783 | 16.529 | 1.00 | 9.49 | A | O |
| ATOM | 3695 | N | PRO | A | 645 | 24.294 | −9.404 | 16.929 | 1.00 | 11.40 | A | N |
| ATOM | 3696 | CA | PRO | A | 645 | 24.890 | −10.317 | 17.908 | 1.00 | 11.04 | A | C |
| ATOM | 3698 | CB | PRO | A | 645 | 26.306 | −9.773 | 18.072 | 1.00 | 11.53 | A | C |
| ATOM | 3701 | CG | PRO | A | 645 | 26.133 | −8.323 | 17.893 | 1.00 | 12.78 | A | C |
| ATOM | 3704 | CD | PRO | A | 645 | 25.098 | −8.180 | 16.770 | 1.00 | 11.64 | A | C |
| ATOM | 3707 | C | PRO | A | 645 | 24.153 | −10.280 | 19.237 | 1.00 | 10.76 | A | C |
| ATOM | 3708 | O | PRO | A | 645 | 23.433 | −9.317 | 19.545 | 1.00 | 10.19 | A | O |
| ATOM | 3709 | N | LYS | A | 646 | 24.334 | −11.309 | 20.050 | 1.00 | 10.57 | A | N |
| ATOM | 3711 | CA | LYS | A | 646 | 23.713 | −11.298 | 21.372 | 1.00 | 11.04 | A | C |
| ATOM | 3713 | CB | LYS | A | 646 | 23.920 | −12.626 | 22.090 | 1.00 | 11.87 | A | C |
| ATOM | 3716 | CG | LYS | A | 646 | 23.106 | −12.693 | 23.388 | 1.00 | 12.94 | A | C |
| ATOM | 3719 | CD | LYS | A | 646 | 23.208 | −14.023 | 24.094 | 1.00 | 15.55 | A | C |
| ATOM | 3722 | CE | LYS | A | 646 | 24.549 | −14.181 | 24.784 | 1.00 | 17.68 | A | C |
| ATOM | 3725 | NZ | LYS | A | 646 | 24.744 | −13.211 | 25.909 | 1.00 | 17.61 | A | N |
| ATOM | 3729 | C | LYS | A | 646 | 24.245 | −10.171 | 22.262 | 1.00 | 11.04 | A | C |
| ATOM | 3730 | O | LYS | A | 646 | 25.455 | −10.098 | 22.512 | 1.00 | 10.00 | A | O |
| ATOM | 3731 | N | PRO | A | 647 | 23.367 | −9.298 | 22.764 | 1.00 | 11.04 | A | N |
| ATOM | 3732 | CA | PRO | A | 647 | 23.795 | −8.304 | 23.753 | 1.00 | 11.70 | A | C |
| ATOM | 3734 | CB | PRO | A | 647 | 22.493 | −7.562 | 24.101 | 1.00 | 11.68 | A | C |
| ATOM | 3737 | CG | PRO | A | 647 | 21.613 | −7.755 | 22.933 | 1.00 | 10.24 | A | C |
| ATOM | 3740 | CD | PRO | A | 647 | 21.939 | −9.134 | 22.407 | 1.00 | 10.94 | A | C |
| ATOM | 3743 | C | PRO | A | 647 | 24.385 | −9.009 | 24.989 | 1.00 | 12.95 | A | C |
| ATOM | 3744 | O | PRO | A | 647 | 23.903 | −10.073 | 25.362 | 1.00 | 13.55 | A | O |
| ATOM | 3745 | N | ASP | A | 648 | 25.404 | −8.419 | 25.596 | 1.00 | 14.13 | A | N |
| ATOM | 3747 | CA | ASP | A | 648 | 26.079 | −9.025 | 26.746 | 1.00 | 15.83 | A | C |
| ATOM | 3749 | CB | ASP | A | 648 | 27.078 | −8.037 | 27.343 | 1.00 | 16.30 | A | C |
| ATOM | 3752 | CG | ASP | A | 648 | 27.957 | −8.685 | 28.387 | 1.00 | 19.77 | A | C |
| ATOM | 3753 | OD1 | ASP | A | 648 | 28.581 | −9.737 | 28.087 | 1.00 | 23.36 | A | O |
| ATOM | 3754 | OD2 | ASP | A | 648 | 28.045 | −8.239 | 29.538 | 1.00 | 22.83 | A | O |
| ATOM | 3755 | C | ASP | A | 648 | 25.132 | −9.530 | 27.867 | 1.00 | 15.47 | A | C |
| ATOM | 3756 | O | ASP | A | 648 | 25.328 | −10.620 | 28.405 | 1.00 | 15.60 | A | O |
| ATOM | 3757 | N | LEU | A | 649 | 24.107 | −8.758 | 28.208 | 1.00 | 15.04 | A | N |
| ATOM | 3759 | CA | LEU | A | 649 | 23.198 | −9.145 | 29.293 | 1.00 | 15.54 | A | C |
| ATOM | 3761 | CB | LEU | A | 649 | 22.786 | −7.917 | 30.102 | 1.00 | 16.16 | A | C |
| ATOM | 3764 | CG | LEU | A | 649 | 23.972 | −7.268 | 30.829 | 1.00 | 18.63 | A | C |
| ATOM | 3766 | CD1 | LEU | A | 649 | 23.508 | −6.172 | 31.752 | 1.00 | 21.84 | A | C |
| ATOM | 3770 | CD2 | LEU | A | 649 | 24.819 | −8.285 | 31.590 | 1.00 | 20.44 | A | C |
| ATOM | 3774 | C | LEU | A | 649 | 21.958 | −9.928 | 28.864 | 1.00 | 15.26 | A | C |
| ATOM | 3775 | O | LEU | A | 649 | 21.143 | −10.329 | 29.702 | 1.00 | 15.43 | A | O |
| ATOM | 3776 | N | CYS | A | 650 | 21.816 | −10.172 | 27.573 | 1.00 | 14.41 | A | N |
| ATOM | 3778 | CA | CYS | A | 650 | 20.722 | −10.976 | 27.078 | 1.00 | 14.24 | A | C |
| ATOM | 3780 | CB | CYS | A | 650 | 20.630 | −10.821 | 25.562 | 1.00 | 14.58 | A | C |
| ATOM | 3783 | SG | CYS | A | 650 | 19.263 | −11.720 | 24.860 | 1.00 | 14.34 | A | S |
| ATOM | 3784 | C | CYS | A | 650 | 20.886 | −12.462 | 27.463 | 1.00 | 14.97 | A | C |
| ATOM | 3785 | O | CYS | A | 650 | 21.918 | −13.062 | 27.147 | 1.00 | 14.59 | A | O |
| ATOM | 3786 | N | PRO | A | 651 | 19.882 | −13.046 | 28.131 | 1.00 | 15.29 | A | N |
| ATOM | 3787 | CA | PRO | A | 651 | 19.881 | −14.480 | 28.453 | 1.00 | 15.51 | A | C |
| ATOM | 3789 | CB | PRO | A | 651 | 18.505 | −14.690 | 29.096 | 1.00 | 15.33 | A | C |
| ATOM | 3792 | CG | PRO | A | 651 | 18.118 | −13.377 | 29.606 | 1.00 | 16.20 | A | C |
| ATOM | 3795 | CD | PRO | A | 651 | 18.647 | −12.395 | 28.606 | 1.00 | 15.45 | A | C |
| ATOM | 3798 | C | PRO | A | 651 | 19.950 | −15.280 | 27.156 | 1.00 | 15.61 | A | C |
| ATOM | 3799 | O | PRO | A | 651 | 19.232 | −14.936 | 26.226 | 1.00 | 13.93 | A | O |
| ATOM | 3800 | N | PRO | A | 652 | 20.796 | −16.304 | 27.069 | 1.00 | 15.46 | A | N |
| ATOM | 3801 | CA | PRO | A | 652 | 20.857 | −17.144 | 25.865 | 1.00 | 15.22 | A | C |
| ATOM | 3803 | CB | PRO | A | 652 | 21.714 | −18.328 | 26.322 | 1.00 | 16.43 | A | C |
| ATOM | 3806 | CG | PRO | A | 652 | 22.606 | −17.704 | 27.397 | 1.00 | 15.77 | A | C |
| ATOM | 3809 | CD | PRO | A | 652 | 21.777 | −16.707 | 28.093 | 1.00 | 16.56 | A | C |
| ATOM | 3812 | C | PRO | A | 652 | 19.492 | −17.602 | 25.303 | 1.00 | 14.66 | A | C |
| ATOM | 3813 | O | PRO | A | 652 | 19.290 | −17.564 | 24.078 | 1.00 | 13.44 | A | O |
| ATOM | 3814 | N | VAL | A | 653 | 18.583 | −18.024 | 26.175 | 1.00 | 14.07 | A | N |
| ATOM | 3816 | CA | VAL | A | 653 | 17.232 | −18.429 | 25.766 | 1.00 | 14.45 | A | C |
| ATOM | 3818 | CB | VAL | A | 653 | 16.422 | −18.968 | 26.990 | 1.00 | 15.04 | A | C |
| ATOM | 3820 | CG1 | VAL | A | 653 | 16.178 | −17.897 | 28.039 | 1.00 | 16.41 | A | C |
| ATOM | 3824 | CG2 | VAL | A | 653 | 15.103 | −19.588 | 26.565 | 1.00 | 16.73 | A | C |
| ATOM | 3828 | C | VAL | A | 653 | 16.476 | −17.297 | 25.056 | 1.00 | 13.12 | A | C |
| ATOM | 3829 | O | VAL | A | 653 | 15.724 | −17.519 | 24.101 | 1.00 | 13.27 | A | O |
| ATOM | 3830 | N | LEU | A | 654 | 16.671 | −16.075 | 25.525 | 1.00 | 12.25 | A | N |
| ATOM | 3832 | CA | LEU | A | 654 | 16.061 | −14.930 | 24.859 | 1.00 | 11.55 | A | C |
| ATOM | 3834 | CB | LEU | A | 654 | 16.133 | −13.699 | 25.762 | 1.00 | 12.25 | A | C |
| ATOM | 3837 | CG | LEU | A | 654 | 15.424 | −12.465 | 25.211 | 1.00 | 11.73 | A | C |
| ATOM | 3839 | CD1 | LEU | A | 654 | 13.992 | −12.763 | 24.836 | 1.00 | 12.71 | A | C |
| ATOM | 3843 | CD2 | LEU | A | 654 | 15.497 | −11.336 | 26.213 | 1.00 | 15.67 | A | C |
| ATOM | 3847 | C | LEU | A | 654 | 16.705 | −14.659 | 23.499 | 1.00 | 11.43 | A | C |
| ATOM | 3848 | O | LEU | A | 654 | 16.017 | −14.299 | 22.532 | 1.00 | 9.70 | A | O |
| ATOM | 3849 | N | TYR | A | 655 | 18.023 | −14.819 | 23.391 | 1.00 | 10.65 | A | N |
| ATOM | 3851 | CA | TYR | A | 655 | 18.650 | −14.626 | 22.089 | 1.00 | 10.82 | A | C |
| ATOM | 3853 | CB | TYR | A | 655 | 20.170 | −14.605 | 22.197 | 1.00 | 10.18 | A | C |
| ATOM | 3856 | CG | TYR | A | 655 | 20.847 | −14.244 | 20.909 | 1.00 | 10.71 | A | C |
| ATOM | 3857 | CD1 | TYR | A | 655 | 20.691 | −12.978 | 20.344 | 1.00 | 9.23 | A | C |
| ATOM | 3859 | CE1 | TYR | A | 655 | 21.328 | −12.648 | 19.126 | 1.00 | 10.02 | A | C |
| ATOM | 3861 | CZ | TYR | A | 655 | 22.107 | −13.582 | 18.493 | 1.00 | 10.45 | A | C |
| ATOM | 3862 | OH | TYR | A | 655 | 22.732 | −13.275 | 17.313 | 1.00 | 11.48 | A | O |
| ATOM | 3864 | CE2 | TYR | A | 655 | 22.257 | −14.848 | 19.030 | 1.00 | 11.87 | A | C |
| ATOM | 3866 | CD2 | TYR | A | 655 | 21.607 | −15.175 | 20.222 | 1.00 | 11.48 | A | C |
| ATOM | 3868 | C | TYR | A | 655 | 18.159 | −15.669 | 21.083 | 1.00 | 11.69 | A | C |
| ATOM | 3869 | O | TYR | A | 655 | 17.945 | −15.356 | 19.924 | 1.00 | 13.15 | A | O |
| ATOM | 3870 | N | THR | A | 656 | 17.913 | −16.896 | 21.538 | 1.00 | 12.50 | A | N |
| ATOM | 3872 | CA | THR | A | 656 | 17.343 | −17.927 | 20.681 | 1.00 | 12.57 | A | C |
| ATOM | 3874 | CB | THR | A | 656 | 17.203 | −19.233 | 21.454 | 1.00 | 12.96 | A | C |
| ATOM | 3876 | OG1 | THR | A | 656 | 18.524 | −19.752 | 21.709 | 1.00 | 13.90 | A | O |
| ATOM | 3878 | CG2 | THR | A | 656 | 16.497 | −20.292 | 20.634 | 1.00 | 13.68 | A | C |
| ATOM | 3882 | C | THR | A | 656 | 16.002 | −17.478 | 20.123 | 1.00 | 12.60 | A | C |
| ATOM | 3883 | O | THR | A | 656 | 15.725 | −17.642 | 18.942 | 1.00 | 12.69 | A | O |
| ATOM | 3884 | N | LEU | A | 657 | 15.196 | −16.876 | 20.968 | 1.00 | 12.41 | A | N |
| ATOM | 3886 | CA | LEU | A | 657 | 13.896 | −16.387 | 20.550 | 1.00 | 13.31 | A | C |
| ATOM | 3888 | CB | LEU | A | 657 | 13.159 | −15.855 | 21.767 | 1.00 | 13.71 | A | C |
| ATOM | 3891 | CG | LEU | A | 657 | 11.651 | −15.834 | 21.701 | 1.00 | 17.10 | A | C |
| ATOM | 3893 | CD1 | LEU | A | 657 | 11.129 | −17.245 | 21.475 | 1.00 | 17.52 | A | C |
| ATOM | 3897 | CD2 | LEU | A | 657 | 11.093 | −15.233 | 22.999 | 1.00 | 17.71 | A | C |
| ATOM | 3901 | C | LEU | A | 657 | 14.035 | −15.306 | 19.478 | 1.00 | 12.69 | A | C |
| ATOM | 3902 | O | LEU | A | 657 | 13.318 | −15.328 | 18.471 | 1.00 | 11.85 | A | O |
| ATOM | 3903 | N | MET | A | 658 | 14.973 | −14.381 | 19.681 | 1.00 | 12.08 | A | N |
| ATOM | 3905 | CA | MET | A | 658 | 15.256 | −13.329 | 18.704 | 1.00 | 12.19 | A | C |
| ATOM | 3907 | CB | MET | A | 658 | 16.394 | −12.406 | 19.158 | 1.00 | 12.40 | A | C |
| ATOM | 3910 | CG | MET | A | 658 | 16.108 | −11.534 | 20.375 | 1.00 | 14.24 | A | C |
| ATOM | 3913 | SD | MET | A | 658 | 17.644 | −10.690 | 20.859 | 1.00 | 17.59 | A | S |
| ATOM | 3914 | CE | MET | A | 658 | 17.109 | −9.824 | 22.311 | 1.00 | 17.80 | A | C |
| ATOM | 3918 | C | MET | A | 658 | 15.622 | −13.928 | 17.361 | 1.00 | 11.84 | A | C |
| ATOM | 3919 | O | MET | A | 658 | 15.138 | −13.476 | 16.317 | 1.00 | 11.75 | A | O |
| ATOM | 3920 | N | THR | A | 659 | 16.467 | −14.964 | 17.370 | 1.00 | 11.94 | A | N |
| ATOM | 3922 | CA | THR | A | 659 | 16.955 | −15.520 | 16.113 | 1.00 | 12.12 | A | C |
| ATOM | 3924 | CB | THR | A | 659 | 18.149 | −16.515 | 16.292 | 1.00 | 12.54 | A | C |
| ATOM | 3926 | OG1 | THR | A | 659 | 17.762 | −17.627 | 17.096 | 1.00 | 16.72 | A | O |
| ATOM | 3928 | CG2 | THR | A | 659 | 19.287 | −15.907 | 17.045 | 1.00 | 12.86 | A | C |
| ATOM | 3932 | C | THR | A | 659 | 15.821 | −16.169 | 15.321 | 1.00 | 11.44 | A | C |
| ATOM | 3933 | O | THR | A | 659 | 15.857 | −16.155 | 14.085 | 1.00 | 11.07 | A | O |
| ATOM | 3934 | N | ARG | A | 660 | 14.828 | −16.730 | 16.026 | 1.00 | 11.23 | A | N |
| ATOM | 3936 | CA | ARG | A | 660 | 13.648 | −17.313 | 15.390 | 1.00 | 11.85 | A | C |
| ATOM | 3938 | CB | ARG | A | 660 | 12.801 | −18.090 | 16.395 | 1.00 | 12.33 | A | C |
| ATOM | 3941 | CG | ARG | A | 660 | 13.442 | −19.396 | 16.829 | 1.00 | 15.41 | A | C |
| ATOM | 3944 | CD | ARG | A | 660 | 12.668 | −20.082 | 17.920 | 1.00 | 18.66 | A | C |
| ATOM | 3947 | NE | ARG | A | 660 | 11.342 | −20.465 | 17.435 | 1.00 | 21.96 | A | N |
| ATOM | 3949 | CZ | ARG | A | 660 | 10.238 | −20.507 | 18.175 | 1.00 | 24.62 | A | C |
| ATOM | 3950 | NH1 | ARG | A | 660 | 10.262 | −20.224 | 19.484 | 1.00 | 25.28 | A | N |
| ATOM | 3953 | NH2 | ARG | A | 660 | 9.104 | −20.872 | 17.604 | 1.00 | 25.04 | A | N |
| ATOM | 3956 | C | ARG | A | 660 | 12.790 | −16.249 | 14.711 | 1.00 | 11.14 | A | C |
| ATOM | 3957 | O | ARG | A | 660 | 12.256 | −16.473 | 13.614 | 1.00 | 11.17 | A | O |
| ATOM | 3958 | N | CYS | A | 661 | 12.695 | −15.082 | 15.345 | 1.00 | 10.45 | A | N |
| ATOM | 3960 | CA | CYS | A | 661 | 12.009 | −13.926 | 14.756 | 1.00 | 10.32 | A | C |
| ATOM | 3962 | CB | CYS | A | 661 | 11.839 | −12.812 | 15.791 | 1.00 | 10.37 | A | C |
| ATOM | 3965 | SG | CYS | A | 661 | 10.842 | −13.203 | 17.247 | 1.00 | 11.06 | A | S |
| ATOM | 3966 | C | CYS | A | 661 | 12.729 | −13.350 | 13.538 | 1.00 | 10.43 | A | C |
| ATOM | 3967 | O | CYS | A | 661 | 12.100 | −12.678 | 12.705 | 1.00 | 10.50 | A | O |
| ATOM | 3968 | N | TRP | A | 662 | 14.036 | −13.597 | 13.427 | 1.00 | 9.89 | A | N |
| ATOM | 3970 | CA | TRP | A | 662 | 14.812 | −13.171 | 12.269 | 1.00 | 9.87 | A | C |
| ATOM | 3972 | CB | TRP | A | 662 | 16.154 | −12.573 | 12.706 | 1.00 | 9.96 | A | C |
| ATOM | 3975 | CG | TRP | A | 662 | 16.045 | −11.379 | 13.624 | 1.00 | 7.99 | A | C |
| ATOM | 3976 | CD1 | TRP | A | 662 | 15.098 | −10.395 | 13.601 | 1.00 | 9.64 | A | C |
| ATOM | 3978 | NE1 | TRP | A | 662 | 15.341 | −9.479 | 14.599 | 1.00 | 9.65 | A | N |
| ATOM | 3980 | CE2 | TRP | A | 662 | 16.458 | −9.867 | 15.292 | 1.00 | 9.01 | A | C |
| ATOM | 3981 | CD2 | TRP | A | 662 | 16.915 | −11.064 | 14.707 | 1.00 | 8.52 | A | C |
| ATOM | 3982 | CE3 | TRP | A | 662 | 18.060 | −11.668 | 15.241 | 1.00 | 8.33 | A | C |
| ATOM | 3984 | CZ3 | TRP | A | 662 | 18.678 | −11.079 | 16.328 | 1.00 | 9.97 | A | C |
| ATOM | 3986 | CH2 | TRP | A | 662 | 18.197 | −9.903 | 16.882 | 1.00 | 9.90 | A | C |
| ATOM | 3988 | CZ2 | TRP | A | 662 | 17.091 | −9.285 | 16.390 | 1.00 | 8.65 | A | C |
| ATOM | 3990 | C | TRP | A | 662 | 15.035 | −14.293 | 11.250 | 1.00 | 10.88 | A | C |
| ATOM | 3991 | O | TRP | A | 662 | 16.003 | −14.279 | 10.518 | 1.00 | 11.46 | A | O |
| ATOM | 3992 | N | ASP | A | 663 | 14.117 | −15.247 | 11.169 | 1.00 | 12.36 | A | N |
| ATOM | 3994 | CA | ASP | A | 663 | 14.145 | −16.205 | 10.079 | 1.00 | 13.31 | A | C |
| ATOM | 3996 | CB | ASP | A | 663 | 13.079 | −17.269 | 10.250 | 1.00 | 13.47 | A | C |
| ATOM | 3999 | CG | ASP | A | 663 | 13.383 | −18.523 | 9.450 | 1.00 | 17.38 | A | C |
| ATOM | 4000 | OD1 | ASP | A | 663 | 13.368 | −18.472 | 8.199 | 1.00 | 19.87 | A | O |
| ATOM | 4001 | OD2 | ASP | A | 663 | 13.623 | −19.611 | 10.004 | 1.00 | 19.38 | A | O |
| ATOM | 4002 | C | ASP | A | 663 | 13.902 | −15.428 | 8.791 | 1.00 | 13.36 | A | C |
| ATOM | 4003 | O | ASP | A | 663 | 13.021 | −14.564 | 8.740 | 1.00 | 12.27 | A | O |
| ATOM | 4004 | N | TYR | A | 664 | 14.708 | −15.693 | 7.772 | 1.00 | 13.88 | A | N |
| ATOM | 4006 | CA | TYR | A | 664 | 14.563 | −14.999 | 6.493 | 1.00 | 14.75 | A | C |
| ATOM | 4008 | CB | TYR | A | 664 | 15.586 | −15.465 | 5.445 | 1.00 | 15.39 | A | C |
| ATOM | 4011 | CG | TYR | A | 664 | 15.757 | −14.437 | 4.351 | 1.00 | 16.86 | A | C |
| ATOM | 4012 | CD1 | TYR | A | 664 | 16.650 | −13.378 | 4.508 | 1.00 | 19.69 | A | C |
| ATOM | 4014 | CE1 | TYR | A | 664 | 16.807 | −12.418 | 3.526 | 1.00 | 21.35 | A | C |
| ATOM | 4016 | CZ | TYR | A | 664 | 16.055 | −12.492 | 2.381 | 1.00 | 21.53 | A | C |
| ATOM | 4017 | OH | TYR | A | 664 | 16.230 | −11.512 | 1.432 | 1.00 | 26.28 | A | O |
| ATOM | 4019 | CE2 | TYR | A | 664 | 15.146 | −13.520 | 2.189 | 1.00 | 21.81 | A | C |
| ATOM | 4021 | CD2 | TYR | A | 664 | 14.992 | −14.490 | 3.181 | 1.00 | 19.31 | A | C |
| ATOM | 4023 | C | TYR | A | 664 | 13.158 | −15.162 | 5.918 | 1.00 | 15.14 | A | C |
| ATOM | 4024 | O | TYR | A | 664 | 12.615 | −14.215 | 5.389 | 1.00 | 15.70 | A | O |
| ATOM | 4025 | N | ASP | A | 665 | 12.602 | −16.366 | 6.020 | 1.00 | 15.67 | A | N |
| ATOM | 4027 | CA | ASP | A | 665 | 11.266 | −16.674 | 5.507 | 1.00 | 16.37 | A | C |
| ATOM | 4029 | CB | ASP | A | 665 | 11.137 | −18.185 | 5.297 | 1.00 | 16.98 | A | C |
| ATOM | 4032 | CG | ASP | A | 665 | 9.894 | −18.570 | 4.536 | 1.00 | 20.27 | A | C |
| ATOM | 4033 | OD1 | ASP | A | 665 | 8.880 | −17.841 | 4.603 | 1.00 | 21.22 | A | O |
| ATOM | 4034 | OD2 | ASP | A | 665 | 9.847 | −19.600 | 3.828 | 1.00 | 25.29 | A | O |
| ATOM | 4035 | C | ASP | A | 665 | 10.197 | −16.222 | 6.508 | 1.00 | 15.72 | A | C |
| ATOM | 4036 | O | ASP | A | 665 | 10.133 | −16.769 | 7.607 | 1.00 | 14.51 | A | O |
| ATOM | 4037 | N | PRO | A | 666 | 9.382 | −15.230 | 6.167 | 1.00 | 16.24 | A | N |
| ATOM | 4038 | CA | PRO | A | 666 | 8.375 | −14.747 | 7.129 | 1.00 | 16.93 | A | C |
| ATOM | 4040 | CB | PRO | A | 666 | 7.583 | −13.690 | 6.346 | 1.00 | 17.41 | A | C |
| ATOM | 4043 | CG | PRO | A | 666 | 8.026 | −13.812 | 4.904 | 1.00 | 17.55 | A | C |
| ATOM | 4046 | CD | PRO | A | 666 | 9.371 | −14.460 | 4.914 | 1.00 | 16.23 | A | C |
| ATOM | 4049 | C | PRO | A | 666 | 7.471 | −15.869 | 7.673 | 1.00 | 17.49 | A | C |
| ATOM | 4050 | O | PRO | A | 666 | 7.117 | −15.846 | 8.857 | 1.00 | 16.21 | A | O |
| ATOM | 4051 | N | SER | A | 667 | 7.161 | −16.855 | 6.823 | 1.00 | 18.52 | A | N |
| ATOM | 4053 | CA | SER | A | 667 | 6.277 | −17.980 | 7.182 | 1.00 | 19.22 | A | C |
| ATOM | 4055 | CB | SER | A | 667 | 6.089 | −18.936 | 5.988 | 1.00 | 19.21 | A | C |
| ATOM | 4058 | OG | SER | A | 667 | 5.265 | −18.349 | 5.018 | 1.00 | 22.33 | A | O |
| ATOM | 4060 | C | SER | A | 667 | 6.774 | −18.786 | 8.355 | 1.00 | 18.58 | A | C |
| ATOM | 4061 | O | SER | A | 667 | 5.977 | −19.427 | 9.028 | 1.00 | 19.88 | A | O |
| ATOM | 4062 | N | ASP | A | 668 | 8.083 | −18.767 | 8.602 | 1.00 | 18.14 | A | N |
| ATOM | 4064 | CA | ASP | A | 668 | 8.691 | −19.514 | 9.690 | 1.00 | 17.77 | A | C |
| ATOM | 4066 | CB | ASP | A | 668 | 10.041 | −20.074 | 9.239 | 1.00 | 18.75 | A | C |
| ATOM | 4069 | CG | ASP | A | 668 | 9.875 | −21.107 | 8.141 | 1.00 | 22.03 | A | C |
| ATOM | 4070 | OD1 | ASP | A | 668 | 10.883 | −21.598 | 7.597 | 1.00 | 27.40 | A | O |
| ATOM | 4071 | OD2 | ASP | A | 668 | 8.741 | −21.464 | 7.754 | 1.00 | 25.19 | A | O |
| ATOM | 4072 | C | ASP | A | 668 | 8.866 | −18.773 | 10.999 | 1.00 | 16.19 | A | C |
| ATOM | 4073 | O | ASP | A | 668 | 9.285 | −19.368 | 11.975 | 1.00 | 16.42 | A | O |
| ATOM | 4074 | N | ARG | A | 669 | 8.544 | −17.484 | 11.041 | 1.00 | 14.15 | A | N |
| ATOM | 4076 | CA | ARG | A | 669 | 8.698 | −16.742 | 12.294 | 1.00 | 12.17 | A | C |
| ATOM | 4078 | CB | ARG | A | 669 | 8.754 | −15.241 | 12.032 | 1.00 | 11.55 | A | C |
| ATOM | 4081 | CG | ARG | A | 669 | 9.876 | −14.823 | 11.139 | 1.00 | 10.93 | A | C |
| ATOM | 4084 | CD | ARG | A | 669 | 9.765 | −13.404 | 10.667 | 1.00 | 8.98 | A | C |
| ATOM | 4087 | NE | ARG | A | 669 | 10.697 | −13.209 | 9.551 | 1.00 | 9.01 | A | N |
| ATOM | 4089 | CZ | ARG | A | 669 | 10.533 | −12.298 | 8.608 | 1.00 | 8.74 | A | C |
| ATOM | 4090 | NH1 | ARG | A | 669 | 9.534 | −11.433 | 8.687 | 1.00 | 9.38 | A | N |
| ATOM | 4093 | NH2 | ARG | A | 669 | 11.399 | −12.224 | 7.601 | 1.00 | 9.94 | A | N |
| ATOM | 4096 | C | ARG | A | 669 | 7.528 | −17.054 | 13.223 | 1.00 | 11.87 | A | C |
| ATOM | 4097 | O | ARG | A | 669 | 6.428 | −17.374 | 12.748 | 1.00 | 11.94 | A | O |
| ATOM | 4098 | N | PRO | A | 670 | 7.721 | −16.936 | 14.531 | 1.00 | 11.24 | A | N |
| ATOM | 4099 | CA | PRO | A | 670 | 6.624 | −17.164 | 15.492 | 1.00 | 10.97 | A | C |
| ATOM | 4101 | CB | PRO | A | 670 | 7.294 | −16.965 | 16.857 | 1.00 | 12.04 | A | C |
| ATOM | 4104 | CG | PRO | A | 670 | 8.752 | −16.960 | 16.597 | 1.00 | 12.70 | A | C |
| ATOM | 4107 | CD | PRO | A | 670 | 8.967 | −16.549 | 15.200 | 1.00 | 11.74 | A | C |
| ATOM | 4110 | C | PRO | A | 670 | 5.502 | −16.141 | 15.365 | 1.00 | 10.78 | A | C |
| ATOM | 4111 | O | PRO | A | 670 | 5.748 | −15.057 | 14.862 | 1.00 | 10.11 | A | O |
| ATOM | 4112 | N | ARG | A | 671 | 4.306 | −16.486 | 15.841 | 1.00 | 10.15 | A | N |
| ATOM | 4114 | CA | ARG | A | 671 | 3.225 | −15.531 | 16.041 | 1.00 | 10.66 | A | C |
| ATOM | 4116 | CB | ARG | A | 671 | 1.913 | −16.274 | 16.265 | 1.00 | 11.28 | A | C |
| ATOM | 4119 | CG | ARG | A | 671 | 1.425 | −17.188 | 15.162 | 1.00 | 14.40 | A | C |
| ATOM | 4122 | CD | ARG | A | 671 | 0.097 | −17.817 | 15.566 | 1.00 | 18.14 | A | C |
| ATOM | 4125 | NE | ARG | A | 671 | −0.602 | −18.573 | 14.531 | 1.00 | 19.61 | A | N |
| ATOM | 4127 | CZ | ARG | A | 671 | −0.460 | −19.884 | 14.309 | 1.00 | 22.54 | A | C |
| ATOM | 4128 | NH1 | ARG | A | 671 | 0.379 | −20.617 | 15.035 | 1.00 | 21.69 | A | N |
| ATOM | 4131 | NH2 | ARG | A | 671 | −1.180 | −20.473 | 13.358 | 1.00 | 23.16 | A | N |
| ATOM | 4134 | C | ARG | A | 671 | 3.478 | −14.710 | 17.304 | 1.00 | 9.70 | A | C |
| ATOM | 4135 | O | ARG | A | 671 | 4.179 | −15.161 | 18.193 | 1.00 | 9.46 | A | O |
| ATOM | 4136 | N | PHE | A | 672 | 2.854 | −13.549 | 17.425 | 1.00 | 9.68 | A | N |
| ATOM | 4138 | CA | PHE | A | 672 | 2.954 | −12.793 | 18.661 | 1.00 | 9.83 | A | C |
| ATOM | 4140 | CB | PHE | A | 672 | 2.376 | −11.377 | 18.529 | 1.00 | 9.02 | A | C |
| ATOM | 4143 | CG | PHE | A | 672 | 3.314 | −10.400 | 17.874 | 1.00 | 7.29 | A | C |
| ATOM | 4144 | CD1 | PHE | A | 672 | 4.499 | −10.034 | 18.499 | 1.00 | 7.29 | A | C |
| ATOM | 4146 | CE1 | PHE | A | 672 | 5.348 | −9.114 | 17.899 | 1.00 | 6.39 | A | C |
| ATOM | 4148 | CZ | PHE | A | 672 | 5.024 | −8.548 | 16.678 | 1.00 | 6.43 | A | C |
| ATOM | 4150 | CE2 | PHE | A | 672 | 3.873 | −8.906 | 16.040 | 1.00 | 7.92 | A | C |
| ATOM | 4152 | CD2 | PHE | A | 672 | 3.008 | −9.841 | 16.639 | 1.00 | 7.47 | A | C |
| ATOM | 4154 | C | PHE | A | 672 | 2.316 | −13.516 | 19.857 | 1.00 | 10.52 | A | C |
| ATOM | 4155 | O | PHE | A | 672 | 2.809 | −13.376 | 20.963 | 1.00 | 10.42 | A | O |
| ATOM | 4156 | N | THR | A | 673 | 1.245 | −14.285 | 19.652 | 1.00 | 11.02 | A | N |
| ATOM | 4158 | CA | THR | A | 673 | 0.664 | −15.049 | 20.764 | 1.00 | 12.24 | A | C |
| ATOM | 4160 | CB | THR | A | 673 | −0.597 | −15.834 | 20.351 | 1.00 | 12.82 | A | C |
| ATOM | 4162 | OG1 | THR | A | 673 | −0.322 | −16.642 | 19.189 | 1.00 | 13.06 | A | O |
| ATOM | 4164 | CG2 | THR | A | 673 | −1.684 | −14.908 | 19.961 | 1.00 | 14.88 | A | C |
| ATOM | 4168 | C | THR | A | 673 | 1.682 | −16.025 | 21.330 | 1.00 | 12.24 | A | C |
| ATOM | 4169 | O | THR | A | 673 | 1.768 | −16.205 | 22.552 | 1.00 | 13.08 | A | O |
| ATOM | 4170 | N | GLU | A | 674 | 2.448 | −16.653 | 20.435 | 1.00 | 11.03 | A | N |
| ATOM | 4172 | CA | GLU | A | 674 | 3.482 | −17.598 | 20.811 | 1.00 | 11.45 | A | C |
| ATOM | 4174 | CB | GLU | A | 674 | 3.971 | −18.366 | 19.575 | 1.00 | 11.48 | A | C |
| ATOM | 4177 | CG | GLU | A | 674 | 2.851 | −19.201 | 18.944 | 1.00 | 12.48 | A | C |
| ATOM | 4180 | CD | GLU | A | 674 | 3.143 | −19.750 | 17.549 | 1.00 | 17.12 | A | C |
| ATOM | 4181 | OE1 | GLU | A | 674 | 4.096 | −19.296 | 16.881 | 1.00 | 15.46 | A | O |
| ATOM | 4182 | OE2 | GLU | A | 674 | 2.370 | −20.657 | 17.120 | 1.00 | 18.19 | A | O |
| ATOM | 4183 | C | GLU | A | 674 | 4.651 | −16.904 | 21.527 | 1.00 | 11.40 | A | C |
| ATOM | 4184 | O | GLU | A | 674 | 5.194 | −17.429 | 22.504 | 1.00 | 12.34 | A | O |
| ATOM | 4185 | N | LEU | A | 675 | 5.006 | −15.714 | 21.070 | 1.00 | 11.03 | A | N |
| ATOM | 4187 | CA | LEU | A | 675 | 6.064 | −14.951 | 21.712 | 1.00 | 10.62 | A | C |
| ATOM | 4189 | CB | LEU | A | 675 | 6.459 | −13.750 | 20.855 | 1.00 | 10.87 | A | C |
| ATOM | 4192 | CG | LEU | A | 675 | 7.212 | −14.093 | 19.574 | 1.00 | 11.12 | A | C |
| ATOM | 4194 | CD1 | LEU | A | 675 | 7.279 | −12.885 | 18.652 | 1.00 | 11.98 | A | C |
| ATOM | 4198 | CD2 | LEU | A | 675 | 8.608 | −14.669 | 19.883 | 1.00 | 12.08 | A | C |
| ATOM | 4202 | C | LEU | A | 675 | 5.674 | −14.489 | 23.125 | 1.00 | 10.64 | A | C |
| ATOM | 4203 | O | LEU | A | 675 | 6.537 | −14.412 | 23.997 | 1.00 | 10.13 | A | O |
| ATOM | 4204 | N | VAL | A | 676 | 4.401 | −14.147 | 23.340 | 1.00 | 10.75 | A | N |
| ATOM | 4206 | CA | VAL | A | 676 | 3.949 | −13.746 | 24.663 | 1.00 | 10.97 | A | C |
| ATOM | 4208 | CB | VAL | A | 676 | 2.481 | −13.353 | 24.703 | 1.00 | 11.26 | A | C |
| ATOM | 4210 | CG1 | VAL | A | 676 | 1.972 | −13.247 | 26.176 | 1.00 | 11.67 | A | C |
| ATOM | 4214 | CG2 | VAL | A | 676 | 2.265 | −12.044 | 23.975 | 1.00 | 11.71 | A | C |
| ATOM | 4218 | C | VAL | A | 676 | 4.193 | −14.922 | 25.623 | 1.00 | 12.06 | A | C |
| ATOM | 4219 | O | VAL | A | 676 | 4.724 | −14.750 | 26.719 | 1.00 | 11.23 | A | O |
| ATOM | 4220 | N | CYS | A | 677 | 3.827 | −16.118 | 25.189 | 1.00 | 12.77 | A | N |
| ATOM | 4222 | CA | CYS | A | 677 | 4.070 | −17.309 | 26.003 | 1.00 | 13.59 | A | C |
| ATOM | 4224 | CB | CYS | A | 677 | 3.486 | −18.526 | 25.322 | 1.00 | 14.78 | A | C |
| ATOM | 4227 | SG | CYS | A | 677 | 1.727 | −18.420 | 25.306 | 1.00 | 23.73 | A | S |
| ATOM | 4228 | C | CYS | A | 677 | 5.544 | −17.543 | 26.310 | 1.00 | 12.70 | A | C |
| ATOM | 4229 | O | CYS | A | 677 | 5.911 | −17.813 | 27.472 | 1.00 | 12.44 | A | O |
| ATOM | 4230 | N | SER | A | 678 | 6.384 | −17.449 | 25.280 | 1.00 | 10.95 | A | N |
| ATOM | 4232 | CA | SER | A | 678 | 7.809 | −17.673 | 25.412 | 1.00 | 11.22 | A | C |
| ATOM | 4234 | CB | SER | A | 678 | 8.501 | −17.631 | 24.052 | 1.00 | 11.49 | A | C |
| ATOM | 4237 | OG | SER | A | 678 | 8.087 | −18.690 | 23.218 | 1.00 | 12.71 | A | O |
| ATOM | 4239 | C | SER | A | 678 | 8.463 | −16.617 | 26.319 | 1.00 | 10.73 | A | C |
| ATOM | 4240 | O | SER | A | 678 | 9.291 | −16.946 | 27.175 | 1.00 | 10.09 | A | O |
| ATOM | 4241 | N | LEU | A | 679 | 8.075 | −15.359 | 26.131 | 1.00 | 10.12 | A | N |
| ATOM | 4243 | CA | LEU | A | 679 | 8.633 | −14.277 | 26.941 | 1.00 | 10.19 | A | C |
| ATOM | 4245 | CB | LEU | A | 679 | 8.272 | −12.920 | 26.386 | 1.00 | 10.05 | A | C |
| ATOM | 4248 | CG | LEU | A | 679 | 9.120 | −12.519 | 25.180 | 1.00 | 10.35 | A | C |
| ATOM | 4250 | CD1 | LEU | A | 679 | 8.594 | −11.187 | 24.639 | 1.00 | 10.06 | A | C |
| ATOM | 4254 | CD2 | LEU | A | 679 | 10.583 | −12.406 | 25.546 | 1.00 | 13.61 | A | C |
| ATOM | 4258 | C | LEU | A | 679 | 8.185 | −14.379 | 28.400 | 1.00 | 10.85 | A | C |
| ATOM | 4259 | O | LEU | A | 679 | 8.975 | −14.105 | 29.293 | 1.00 | 10.86 | A | O |
| ATOM | 4260 | N | SER | A | 680 | 6.944 | −14.812 | 28.632 | 1.00 | 11.33 | A | N |
| ATOM | 4262 | CA | SER | A | 680 | 6.442 | −15.009 | 29.976 | 1.00 | 11.30 | A | C |
| ATOM | 4264 | CB | SER | A | 680 | 4.975 | −15.458 | 29.955 | 1.00 | 11.82 | A | C |
| ATOM | 4267 | OG | SER | A | 680 | 4.160 | −14.393 | 29.529 | 1.00 | 13.70 | A | O |
| ATOM | 4269 | C | SER | A | 680 | 7.294 | −16.054 | 30.696 | 1.00 | 11.10 | A | C |
| ATOM | 4270 | O | SER | A | 680 | 7.569 | −15.914 | 31.876 | 1.00 | 10.06 | A | O |
| ATOM | 4271 | N | ASP | A | 681 | 7.709 | −17.079 | 29.965 | 1.00 | 11.27 | A | N |
| ATOM | 4273 | CA | ASP | A | 681 | 8.555 | −18.151 | 30.489 | 1.00 | 11.79 | A | C |
| ATOM | 4275 | CB | ASP | A | 681 | 8.582 | −19.302 | 29.480 | 1.00 | 12.73 | A | C |
| ATOM | 4278 | CG | ASP | A | 681 | 9.205 | −20.567 | 30.017 | 1.00 | 16.09 | A | C |
| ATOM | 4279 | OD1 | ASP | A | 681 | 9.063 | −20.869 | 31.226 | 1.00 | 18.24 | A | O |
| ATOM | 4280 | OD2 | ASP | A | 681 | 9.823 | −21.351 | 29.260 | 1.00 | 19.49 | A | O |
| ATOM | 4281 | C | ASP | A | 681 | 9.962 | −17.637 | 30.793 | 1.00 | 11.36 | A | C |
| ATOM | 4282 | O | ASP | A | 681 | 10.524 | −17.962 | 31.832 | 1.00 | 10.57 | A | O |
| ATOM | 4283 | N | VAL | A | 682 | 10.514 | −16.805 | 29.911 | 1.00 | 10.17 | A | N |
| ATOM | 4285 | CA | VAL | A | 682 | 11.831 | −16.189 | 30.137 | 1.00 | 10.71 | A | C |
| ATOM | 4287 | CB | VAL | A | 682 | 12.323 | −15.421 | 28.879 | 1.00 | 11.06 | A | C |
| ATOM | 4289 | CG1 | VAL | A | 682 | 13.585 | −14.610 | 29.186 | 1.00 | 13.37 | A | C |
| ATOM | 4293 | CG2 | VAL | A | 682 | 12.586 | −16.394 | 27.719 | 1.00 | 12.19 | A | C |
| ATOM | 4297 | C | VAL | A | 682 | 11.784 | −15.270 | 31.371 | 1.00 | 10.10 | A | C |
| ATOM | 4298 | O | VAL | A | 682 | 12.698 | −15.271 | 32.194 | 1.00 | 9.87 | A | O |
| ATOM | 4299 | N | TYR | A | 683 | 10.713 | −14.501 | 31.498 | 1.00 | 9.76 | A | N |
| ATOM | 4301 | CA | TYR | A | 683 | 10.535 | −13.588 | 32.615 | 1.00 | 10.41 | A | C |
| ATOM | 4303 | CB | TYR | A | 683 | 9.258 | −12.801 | 32.428 | 1.00 | 10.16 | A | C |
| ATOM | 4306 | CG | TYR | A | 683 | 8.986 | −11.749 | 33.480 | 1.00 | 11.77 | A | C |
| ATOM | 4307 | CD1 | TYR | A | 683 | 9.973 | −10.837 | 33.858 | 1.00 | 12.15 | A | C |
| ATOM | 4309 | CE1 | TYR | A | 683 | 9.719 | −9.862 | 34.816 | 1.00 | 14.98 | A | C |
| ATOM | 4311 | CZ | TYR | A | 683 | 8.480 | −9.787 | 35.386 | 1.00 | 15.15 | A | C |
| ATOM | 4312 | OH | TYR | A | 683 | 8.225 | −8.818 | 36.312 | 1.00 | 16.29 | A | O |
| ATOM | 4314 | CE2 | TYR | A | 683 | 7.477 | −10.669 | 35.022 | 1.00 | 16.42 | A | C |
| ATOM | 4316 | CD2 | TYR | A | 683 | 7.743 | −11.648 | 34.070 | 1.00 | 13.80 | A | C |
| ATOM | 4318 | C | TYR | A | 683 | 10.482 | −14.356 | 33.933 | 1.00 | 10.77 | A | C |
| ATOM | 4319 | O | TYR | A | 683 | 11.155 | −13.994 | 34.896 | 1.00 | 10.66 | A | O |
| ATOM | 4320 | N | GLN | A | 684 | 9.701 | −15.438 | 33.952 | 1.00 | 11.00 | A | N |
| ATOM | 4322 | CA | GLN | A | 684 | 9.618 | −16.300 | 35.132 | 1.00 | 11.22 | A | C |
| ATOM | 4324 | CB | GLN | A | 684 | 8.629 | −17.444 | 34.930 | 1.00 | 11.31 | A | C |
| ATOM | 4327 | CG | GLN | A | 684 | 8.375 | −18.269 | 36.236 | 1.00 | 13.65 | A | C |
| ATOM | 4330 | CD | GLN | A | 684 | 7.820 | −17.402 | 37.374 | 1.00 | 15.94 | A | C |
| ATOM | 4331 | OE1 | GLN | A | 684 | 8.477 | −17.197 | 38.403 | 1.00 | 19.21 | A | O |
| ATOM | 4332 | NE2 | GLN | A | 684 | 6.630 | −16.887 | 37.180 | 1.00 | 16.15 | A | N |
| ATOM | 4335 | C | GLN | A | 684 | 10.971 | −16.890 | 35.468 | 1.00 | 11.59 | A | C |
| ATOM | 4336 | O | GLN | A | 684 | 11.344 | −16.943 | 36.636 | 1.00 | 11.70 | A | O |
| ATOM | 4337 | N | MET | A | 685 | 11.690 | −17.356 | 34.453 | 1.00 | 12.42 | A | N |
| ATOM | 4339 | CA | MET | A | 685 | 13.022 | −17.913 | 34.646 | 1.00 | 14.85 | A | C |
| ATOM | 4341 | CB | MET | A | 685 | 13.616 | −18.457 | 33.334 | 1.00 | 15.51 | A | C |
| ATOM | 4344 | CG | MET | A | 685 | 15.075 | −18.894 | 33.413 | 1.00 | 20.31 | A | C |
| ATOM | 4347 | SD | MET | A | 685 | 16.347 | −17.557 | 33.428 | 1.00 | 30.38 | A | S |
| ATOM | 4348 | CE | MET | A | 685 | 16.971 | −17.643 | 31.702 | 1.00 | 29.94 | A | C |
| ATOM | 4352 | C | MET | A | 685 | 13.952 | −16.870 | 35.251 | 1.00 | 14.79 | A | C |
| ATOM | 4353 | O | MET | A | 685 | 14.707 | −17.201 | 36.149 | 1.00 | 14.67 | A | O |
| ATOM | 4354 | N | GLU | A | 686 | 13.908 | −15.623 | 34.767 | 1.00 | 15.09 | A | N |
| ATOM | 4356 | CA | GLU | A | 686 | 14.782 | −14.564 | 35.321 | 1.00 | 15.60 | A | C |
| ATOM | 4358 | CB | GLU | A | 686 | 14.765 | −13.293 | 34.457 | 1.00 | 15.66 | A | C |
| ATOM | 4361 | CG | GLU | A | 686 | 15.520 | −13.394 | 33.143 | 1.00 | 18.58 | A | C |
| ATOM | 4364 | CD | GLU | A | 686 | 17.018 | −13.650 | 33.305 | 1.00 | 19.75 | A | C |
| ATOM | 4365 | OE1 | GLU | A | 686 | 17.692 | −12.967 | 34.110 | 1.00 | 19.12 | A | O |
| ATOM | 4366 | OE2 | GLU | A | 686 | 17.521 | −14.545 | 32.603 | 1.00 | 25.71 | A | O |
| ATOM | 4367 | C | GLU | A | 686 | 14.431 | −14.212 | 36.776 | 1.00 | 15.84 | A | C |
| ATOM | 4368 | O | GLU | A | 686 | 15.319 | −13.886 | 37.568 | 1.00 | 15.94 | A | O |
| ATOM | 4369 | N | LYS | A | 687 | 13.146 | −14.251 | 37.124 | 1.00 | 15.72 | A | N |
| ATOM | 4371 | CA | LYS | A | 687 | 12.727 | −14.031 | 38.499 | 1.00 | 16.97 | A | C |
| ATOM | 4373 | CB | LYS | A | 687 | 11.206 | −13.895 | 38.594 | 1.00 | 16.88 | A | C |
| ATOM | 4376 | CG | LYS | A | 687 | 10.707 | −12.578 | 38.036 | 1.00 | 17.92 | A | C |
| ATOM | 4379 | CD | LYS | A | 687 | 9.303 | −12.243 | 38.452 | 1.00 | 19.62 | A | C |
| ATOM | 4382 | CE | LYS | A | 687 | 8.270 | −13.060 | 37.692 | 1.00 | 20.81 | A | C |
| ATOM | 4385 | NZ | LYS | A | 687 | 6.878 | −12.646 | 38.085 | 1.00 | 20.18 | A | N |
| ATOM | 4389 | C | LYS | A | 687 | 13.225 | −15.171 | 39.407 | 1.00 | 17.45 | A | C |
| ATOM | 4390 | O | LYS | A | 687 | 13.629 | −14.925 | 40.534 | 1.00 | 18.25 | A | O |
| ATOM | 4391 | N | ASP | A | 688 | 13.214 | −16.399 | 38.900 | 1.00 | 18.00 | A | N |
| ATOM | 4393 | CA | ASP | A | 688 | 13.607 | −17.573 | 39.679 | 1.00 | 19.39 | A | C |
| ATOM | 4395 | CB | ASP | A | 688 | 13.129 | −18.859 | 38.991 | 1.00 | 19.60 | A | C |
| ATOM | 4398 | CG | ASP | A | 688 | 11.616 | −19.005 | 39.009 | 1.00 | 20.07 | A | C |
| ATOM | 4399 | OD1 | ASP | A | 688 | 10.939 | −18.210 | 39.683 | 1.00 | 18.55 | A | O |
| ATOM | 4400 | OD2 | ASP | A | 688 | 11.017 | −19.885 | 38.363 | 1.00 | 21.49 | A | O |
| ATOM | 4401 | C | ASP | A | 688 | 15.114 | −17.677 | 39.938 | 1.00 | 20.75 | A | C |
| ATOM | 4402 | O | ASP | A | 688 | 15.519 | −18.280 | 40.936 | 1.00 | 20.21 | A | O |
| ATOM | 4403 | N | ILE | A | 689 | 15.932 | −17.094 | 39.064 | 1.00 | 22.34 | A | N |
| ATOM | 4405 | CA | ILE | A | 689 | 17.401 | −17.136 | 39.206 | 1.00 | 24.85 | A | C |
| ATOM | 4407 | CB | ILE | A | 689 | 18.096 | −17.337 | 37.831 | 1.00 | 25.22 | A | C |
| ATOM | 4409 | CG1 | ILE | A | 689 | 18.027 | −16.062 | 36.982 | 1.00 | 24.97 | A | C |
| ATOM | 4412 | CD1 | ILE | A | 689 | 18.969 | −16.063 | 35.816 | 1.00 | 26.04 | A | C |
| ATOM | 4416 | CG2 | ILE | A | 689 | 17.485 | −18.515 | 37.100 | 1.00 | 26.30 | A | C |
| ATOM | 4420 | C | ILE | A | 689 | 17.989 | −15.894 | 39.845 | 1.00 | 26.98 | A | C |
| ATOM | 4421 | O | ILE | A | 689 | 19.220 | −15.772 | 39.945 | 1.00 | 27.14 | A | O |
| ATOM | 4422 | N | ALA | A | 690 | 17.122 | −14.964 | 40.247 | 1.00 | 29.52 | A | N |
| ATOM | 4424 | CA | ALA | A | 690 | 17.543 | −13.708 | 40.840 | 1.00 | 31.03 | A | C |
| ATOM | 4426 | CB | ALA | A | 690 | 16.576 | −12.605 | 40.460 | 1.00 | 31.13 | A | C |
| ATOM | 4430 | C | ALA | A | 690 | 17.629 | −13.834 | 42.357 | 1.00 | 32.63 | A | C |
| ATOM | 4431 | O | ALA | A | 690 | 16.599 | −13.900 | 43.042 | 1.00 | 32.75 | A | O |
| ATOM | 4432 | N | MET | A | 691 | 18.863 | −13.859 | 42.864 | 1.00 | 34.71 | A | N |
| ATOM | 4434 | CA | MET | A | 691 | 19.159 | −13.824 | 44.304 | 1.00 | 35.61 | A | C |
| ATOM | 4436 | CB | MET | A | 691 | 18.556 | −12.572 | 44.955 | 1.00 | 36.56 | A | C |
| ATOM | 4439 | CG | MET | A | 691 | 19.081 | −11.260 | 44.382 | 1.00 | 39.02 | A | C |
| ATOM | 4442 | SD | MET | A | 691 | 18.165 | −9.844 | 45.010 | 1.00 | 44.70 | A | S |
| ATOM | 4443 | CE | MET | A | 691 | 16.654 | −10.005 | 44.062 | 1.00 | 44.31 | A | C |
| ATOM | 4447 | C | MET | A | 691 | 18.685 | −15.085 | 45.012 | 1.00 | 35.54 | A | C |
| ATOM | 4448 | O | MET | A | 691 | 18.582 | −16.116 | 44.329 | 1.00 | 35.63 | A | O |
| ATOM | 4449 | OXT | MET | A | 691 | 18.437 | −15.031 | 46.228 | 1.00 | 34.84 | A | O |
| ATOM | 4450 | O1A | ANP | L | 1 | 5.879 | 14.129 | 17.474 | 1.00 | 56.03 | L | O |
| ATOM | 4451 | PA | ANP | L | 1 | 5.828 | 13.237 | 18.779 | 1.00 | 54.99 | L | P |
| ATOM | 4452 | O2A | ANP | L | 1 | 4.359 | 12.664 | 18.974 | 1.00 | 55.47 | L | O |
| ATOM | 4454 | O3A | ANP | L | 1 | 6.928 | 12.057 | 18.803 | 1.00 | 58.70 | L | O |
| ATOM | 4455 | PB | ANP | L | 1 | 7.534 | 11.319 | 17.499 | 1.00 | 61.85 | L | P |
| ATOM | 4456 | O1B | ANP | L | 1 | 6.806 | 11.789 | 16.164 | 1.00 | 60.90 | L | O |
| ATOM | 4457 | O2B | ANP | L | 1 | 7.338 | 9.749 | 17.688 | 1.00 | 61.90 | L | O |
| ATOM | 4459 | N3B | ANP | L | 1 | 9.254 | 11.643 | 17.401 | 1.00 | 63.51 | L | N |
| ATOM | 4461 | PG | ANP | L | 1 | 9.852 | 13.230 | 16.957 | 1.00 | 65.53 | L | P |
| ATOM | 4462 | O3G | ANP | L | 1 | 10.884 | 13.100 | 15.748 | 1.00 | 66.11 | L | O |
| ATOM | 4464 | O2G | ANP | L | 1 | 10.586 | 13.911 | 18.193 | 1.00 | 64.86 | L | O |
| ATOM | 4466 | O1G | ANP | L | 1 | 8.643 | 14.135 | 16.458 | 1.00 | 65.59 | L | O |
| ATOM | 4467 | O5* | ANP | L | 1 | 6.277 | 14.111 | 20.050 | 1.00 | 51.13 | L | O |
| ATOM | 4468 | C5* | ANP | L | 1 | 7.549 | 14.739 | 20.107 | 1.00 | 46.73 | L | C |
| ATOM | 4471 | C4* | ANP | L | 1 | 8.168 | 14.467 | 21.461 | 1.00 | 44.62 | L | C |
| ATOM | 4473 | O4* | ANP | L | 1 | 7.254 | 14.809 | 22.510 | 1.00 | 41.60 | L | O |
| ATOM | 4474 | C1* | ANP | L | 1 | 7.281 | 13.799 | 23.515 | 1.00 | 38.50 | L | C |
| ATOM | 4476 | C2* | ANP | L | 1 | 8.155 | 12.643 | 23.037 | 1.00 | 42.11 | L | C |
| ATOM | 4478 | O2* | ANP | L | 1 | 9.304 | 12.486 | 23.860 | 1.00 | 44.90 | L | O |
| ATOM | 4480 | C3* | ANP | L | 1 | 8.524 | 12.998 | 21.614 | 1.00 | 43.96 | L | C |
| ATOM | 4482 | O3* | ANP | L | 1 | 9.901 | 12.797 | 21.351 | 1.00 | 45.53 | L | O |
| ATOM | 4484 | N9 | ANP | L | 1 | 5.951 | 13.197 | 23.740 | 1.00 | 31.30 | L | N |
| ATOM | 4485 | C8 | ANP | L | 1 | 5.015 | 12.876 | 22.819 | 1.00 | 28.19 | L | C |
| ATOM | 4487 | N7 | ANP | L | 1 | 3.913 | 12.295 | 23.381 | 1.00 | 25.49 | L | N |
| ATOM | 4488 | C5 | ANP | L | 1 | 4.161 | 12.228 | 24.694 | 1.00 | 24.63 | L | C |
| ATOM | 4489 | C6 | ANP | L | 1 | 3.506 | 11.746 | 25.918 | 1.00 | 21.30 | L | C |
| ATOM | 4490 | N6 | ANP | L | 1 | 2.277 | 11.204 | 25.779 | 1.00 | 18.20 | L | N |
| ATOM | 4493 | C4 | ANP | L | 1 | 5.475 | 12.793 | 24.898 | 1.00 | 26.86 | L | C |
| ATOM | 4494 | N3 | ANP | L | 1 | 6.067 | 12.897 | 26.207 | 1.00 | 23.82 | L | N |
| ATOM | 4495 | C2 | ANP | L | 1 | 5.359 | 12.433 | 27.261 | 1.00 | 22.61 | L | C |
| ATOM | 4497 | N1 | ANP | L | 1 | 4.129 | 11.883 | 27.114 | 1.00 | 19.81 | L | N |
| ATOM | 4498 | O | HOH | M | 1 | 11.906 | 10.877 | 19.048 | 1.00 | 54.80 | M | O |
| ATOM | 4501 | O | HOH | W | 1 | 19.443 | −9.552 | 13.290 | 1.00 | 12.78 | W | O |
| ATOM | 4504 | O | HOH | W | 2 | 24.437 | −5.004 | 22.245 | 1.00 | 16.30 | W | O |
| ATOM | 4507 | O | HOH | W | 3 | 16.956 | −5.546 | 11.116 | 1.00 | 14.32 | W | O |
| ATOM | 4510 | O | HOH | W | 4 | 23.654 | −5.882 | 27.256 | 1.00 | 16.76 | W | O |
| ATOM | 4513 | O | HOH | W | 5 | 19.773 | −2.500 | 2.719 | 1.00 | 17.82 | W | O |
| ATOM | 4516 | O | HOH | W | 6 | 14.677 | 3.342 | 18.254 | 1.00 | 12.86 | W | O |
| ATOM | 4519 | O | HOH | W | 7 | 15.460 | −2.754 | 37.319 | 1.00 | 19.09 | W | O |
| ATOM | 4522 | O | HOH | W | 8 | 0.917 | −6.788 | 13.617 | 1.00 | 19.87 | W | O |
| ATOM | 4525 | O | HOH | W | 9 | 6.262 | −4.291 | 9.880 | 1.00 | 17.35 | W | O |
| ATOM | 4528 | O | HOH | W | 10 | −8.084 | −9.594 | 20.676 | 1.00 | 19.55 | W | O |
| ATOM | 4531 | O | HOH | W | 11 | 9.378 | −6.760 | 37.711 | 1.00 | 23.16 | W | O |
| ATOM | 4534 | O | HOH | W | 12 | −4.324 | 2.362 | 25.030 | 1.00 | 17.21 | W | O |
| ATOM | 4537 | O | HOH | W | 13 | 2.631 | −4.978 | 2.213 | 1.00 | 21.67 | W | O |
| ATOM | 4540 | O | HOH | W | 14 | 13.357 | 9.278 | 28.253 | 1.00 | 18.85 | W | O |
| ATOM | 4543 | O | HOH | W | 15 | 18.009 | 2.532 | 9.354 | 1.00 | 17.12 | W | O |
| ATOM | 4546 | O | HOH | W | 16 | 17.534 | −9.551 | 37.009 | 1.00 | 20.96 | W | O |
| ATOM | 4549 | O | HOH | W | 17 | 27.129 | −8.112 | 21.598 | 1.00 | 20.80 | W | O |
| ATOM | 4552 | O | HOH | W | 18 | 18.189 | −15.606 | 12.347 | 1.00 | 21.79 | W | O |
| ATOM | 4555 | O | HOH | W | 19 | 13.177 | 0.863 | 35.291 | 1.00 | 23.40 | W | O |
| ATOM | 4558 | O | HOH | W | 20 | 17.054 | −4.703 | 8.729 | 1.00 | 17.25 | W | O |
| ATOM | 4561 | O | HOH | W | 21 | 7.866 | −18.942 | 20.500 | 1.00 | 24.06 | W | O |
| ATOM | 4564 | O | HOH | W | 22 | 14.700 | 7.035 | 22.806 | 1.00 | 16.93 | W | O |
| ATOM | 4567 | O | HOH | W | 23 | −2.679 | 2.607 | 28.131 | 1.00 | 23.11 | W | O |
| ATOM | 4570 | O | HOH | W | 24 | 6.040 | −9.803 | 37.824 | 1.00 | 33.92 | W | O |
| ATOM | 4573 | O | HOH | W | 25 | 10.004 | −5.316 | 9.507 | 1.00 | 23.61 | W | O |
| ATOM | 4576 | O | HOH | W | 26 | 25.491 | −5.320 | 24.819 | 1.00 | 23.18 | W | O |
| ATOM | 4579 | O | HOH | W | 27 | 4.534 | 32.921 | 12.514 | 1.00 | 20.02 | W | O |
| ATOM | 4582 | O | HOH | W | 28 | 21.903 | −11.752 | 31.783 | 1.00 | 27.60 | W | O |
| ATOM | 4585 | O | HOH | W | 29 | −2.817 | −12.294 | 18.585 | 1.00 | 19.69 | W | O |
| ATOM | 4588 | O | HOH | W | 30 | 0.619 | 25.243 | 18.899 | 1.00 | 22.14 | W | O |
| ATOM | 4591 | O | HOH | W | 31 | 4.508 | −2.772 | −1.220 | 1.00 | 21.13 | W | O |
| ATOM | 4594 | O | HOH | W | 32 | 26.872 | −11.629 | 24.125 | 1.00 | 24.32 | W | O |
| ATOM | 4597 | O | HOH | W | 33 | 26.063 | −13.556 | 19.248 | 1.00 | 23.37 | W | O |
| ATOM | 4600 | O | HOH | W | 34 | −4.463 | 26.591 | 28.285 | 1.00 | 28.20 | W | O |
| ATOM | 4603 | O | HOH | W | 35 | −0.377 | −9.432 | 8.082 | 1.00 | 25.76 | W | O |
| ATOM | 4606 | O | HOH | W | 36 | 11.357 | 4.915 | 15.323 | 1.00 | 31.17 | W | O |
| ATOM | 4609 | O | HOH | W | 37 | 0.444 | −0.589 | 8.490 | 1.00 | 27.25 | W | O |
| ATOM | 4612 | O | HOH | W | 38 | 12.025 | −12.305 | 3.518 | 1.00 | 20.13 | W | O |
| ATOM | 4615 | O | HOH | W | 39 | −7.592 | 21.532 | 34.079 | 1.00 | 24.16 | W | O |
| ATOM | 4618 | O | HOH | W | 40 | −3.034 | 25.675 | 20.623 | 1.00 | 27.86 | W | O |
| ATOM | 4621 | O | HOH | W | 41 | 19.252 | −18.574 | 29.007 | 1.00 | 24.11 | W | O |
| ATOM | 4624 | O | HOH | W | 42 | 18.447 | 5.050 | 22.545 | 1.00 | 26.98 | W | O |
| ATOM | 4627 | O | HOH | W | 43 | 18.236 | 2.548 | 19.150 | 1.00 | 23.34 | W | O |
| ATOM | 4630 | O | HOH | W | 44 | 23.599 | −4.581 | 0.730 | 1.00 | 23.86 | W | O |
| ATOM | 4633 | O | HOH | W | 45 | 5.670 | −14.733 | 33.566 | 1.00 | 22.95 | W | O |
| ATOM | 4636 | O | HOH | W | 46 | 14.080 | −19.737 | 23.406 | 1.00 | 22.24 | W | O |
| ATOM | 4639 | O | HOH | W | 47 | 22.734 | 4.890 | 34.891 | 1.00 | 26.97 | W | O |
| ATOM | 4642 | O | HOH | W | 48 | −3.602 | 22.418 | 16.052 | 1.00 | 29.22 | W | O |
| ATOM | 4645 | O | HOH | W | 49 | 25.830 | −12.392 | 15.030 | 1.00 | 32.70 | W | O |
| ATOM | 4648 | O | HOH | W | 50 | −2.320 | −15.117 | 16.246 | 1.00 | 28.09 | W | O |
| ATOM | 4651 | O | HOH | W | 51 | 2.614 | −23.096 | 16.430 | 1.00 | 31.67 | W | O |
| ATOM | 4654 | O | HOH | W | 52 | 17.362 | −18.998 | 43.191 | 1.00 | 26.88 | W | O |
| ATOM | 4657 | O | HOH | W | 53 | 26.474 | −8.992 | 12.337 | 1.00 | 31.87 | W | O |
| ATOM | 4660 | O | HOH | W | 54 | 16.830 | −4.474 | −1.805 | 1.00 | 36.41 | W | O |
| ATOM | 4663 | O | HOH | W | 55 | 4.947 | −4.071 | 36.311 | 1.00 | 25.83 | W | O |
| ATOM | 4666 | O | HOH | W | 56 | 1.631 | 1.495 | 10.141 | 1.00 | 25.82 | W | O |
| ATOM | 4669 | O | HOH | W | 57 | 21.157 | −1.727 | −0.971 | 1.00 | 32.21 | W | O |
| ATOM | 4672 | O | HOH | W | 58 | 20.249 | −8.173 | 36.530 | 1.00 | 21.67 | W | O |
| ATOM | 4675 | O | HOH | W | 59 | −2.610 | −9.023 | 9.489 | 1.00 | 35.63 | W | O |
| ATOM | 4678 | O | HOH | W | 60 | −9.404 | 23.395 | 33.556 | 1.00 | 27.23 | W | O |
| ATOM | 4681 | O | HOH | W | 61 | 11.153 | 3.802 | −0.793 | 1.00 | 32.48 | W | O |
| ATOM | 4684 | O | HOH | W | 62 | 2.156 | −13.178 | 30.950 | 1.00 | 32.64 | W | O |
| ATOM | 4687 | O | HOH | W | 63 | 15.393 | −13.901 | 45.580 | 1.00 | 34.94 | W | O |
| ATOM | 4690 | O | HOH | W | 64 | 5.734 | −20.060 | 12.705 | 1.00 | 28.43 | W | O |
| ATOM | 4693 | O | HOH | W | 65 | −0.390 | 18.866 | 1.150 | 1.00 | 37.04 | W | O |
| ATOM | 4696 | O | HOH | W | 66 | 9.308 | 23.821 | 20.537 | 1.00 | 33.19 | W | O |
| ATOM | 4699 | O | HOH | W | 67 | −8.657 | −12.592 | 15.980 | 1.00 | 30.95 | W | O |
| ATOM | 4702 | O | HOH | W | 68 | 6.009 | −10.288 | 5.499 | 1.00 | 30.71 | W | O |
| ATOM | 4705 | O | HOH | W | 69 | 5.849 | −14.630 | 36.206 | 1.00 | 36.64 | W | O |
| ATOM | 4708 | O | HOH | W | 70 | −0.379 | −15.926 | 24.303 | 1.00 | 26.34 | W | O |
| ATOM | 4711 | O | HOH | W | 71 | 19.013 | 6.091 | 37.596 | 1.00 | 29.23 | W | O |
| ATOM | 4714 | O | HOH | W | 72 | −2.437 | −8.935 | 24.621 | 1.00 | 26.00 | W | O |
| ATOM | 4717 | O | HOH | W | 73 | 13.274 | 10.054 | 0.171 | 1.00 | 32.22 | W | O |
| ATOM | 4720 | O | HOH | W | 74 | 11.266 | −4.798 | 37.300 | 1.00 | 27.56 | W | O |
| ATOM | 4723 | O | HOH | W | 75 | 18.634 | 5.650 | 3.509 | 1.00 | 30.22 | W | O |
| ATOM | 4726 | O | HOH | W | 76 | 17.088 | −17.411 | 8.048 | 1.00 | 26.93 | W | O |
| ATOM | 4729 | O | HOH | W | 77 | 23.077 | 3.880 | 2.721 | 1.00 | 25.25 | W | O |
| ATOM | 4732 | O | HOH | W | 78 | 24.631 | 5.289 | 28.100 | 1.00 | 53.16 | W | O |
| ATOM | 4735 | O | HOH | W | 79 | 10.896 | −4.046 | −5.174 | 1.00 | 30.72 | W | O |
| ATOM | 4738 | O | HOH | W | 80 | 21.483 | −18.240 | 22.299 | 1.00 | 31.63 | W | O |
| ATOM | 4741 | O | HOH | W | 81 | 17.664 | −12.362 | 36.744 | 1.00 | 27.21 | W | O |
| ATOM | 4744 | O | HOH | W | 82 | −6.800 | −5.096 | 9.419 | 1.00 | 31.32 | W | O |
| ATOM | 4747 | O | HOH | W | 83 | 3.254 | −6.214 | 33.543 | 1.00 | 29.36 | W | O |
| ATOM | 4750 | O | HOH | W | 84 | −6.655 | 5.297 | 23.427 | 1.00 | 38.97 | W | O |
| ATOM | 4753 | O | HOH | W | 85 | 6.119 | 3.003 | 3.176 | 1.00 | 39.55 | W | O |
| ATOM | 4756 | O | HOH | W | 86 | 16.440 | 5.449 | 7.469 | 1.00 | 35.83 | W | O |
| ATOM | 4759 | O | HOH | W | 87 | −6.743 | 15.088 | 41.223 | 1.00 | 33.66 | W | O |
| ATOM | 4762 | O | HOH | W | 88 | −8.714 | −10.654 | 12.475 | 1.00 | 45.30 | W | O |
| ATOM | 4765 | O | HOH | W | 89 | 5.455 | 6.147 | 14.560 | 1.00 | 34.25 | W | O |
| ATOM | 4768 | O | HOH | W | 90 | 7.863 | −2.178 | 41.320 | 1.00 | 41.21 | W | O |
| ATOM | 4771 | O | HOH | W | 91 | 3.037 | −8.511 | 31.787 | 1.00 | 43.98 | W | O |
| ATOM | 4774 | O | HOH | W | 92 | −5.700 | 2.904 | 27.490 | 1.00 | 30.78 | W | O |
| ATOM | 4777 | O | HOH | W | 93 | 10.698 | −16.094 | 41.240 | 1.00 | 40.42 | W | O |
| ATOM | 4780 | O | HOH | W | 94 | 8.297 | −21.654 | 26.890 | 1.00 | 35.50 | W | O |
| ATOM | 4783 | O | HOH | W | 95 | −7.043 | 26.092 | 27.806 | 1.00 | 33.08 | W | O |
| ATOM | 4786 | O | HOH | W | 96 | 12.953 | −20.340 | 21.075 | 1.00 | 26.90 | W | O |
| ATOM | 4789 | O | HOH | W | 97 | −1.020 | 6.390 | 14.791 | 1.00 | 42.20 | W | O |
| ATOM | 4792 | O | HOH | W | 98 | 9.903 | −2.480 | 37.263 | 1.00 | 39.90 | W | O |
| ATOM | 4795 | O | HOH | W | 99 | −14.690 | 5.403 | 20.304 | 1.00 | 43.77 | W | O |
| ATOM | 4798 | O | HOH | W | 100 | 7.497 | −20.614 | 33.258 | 1.00 | 42.00 | W | O |
| ATOM | 4801 | O | HOH | W | 101 | −6.310 | −0.802 | 28.924 | 1.00 | 38.73 | W | O |
| ATOM | 4804 | O | HOH | W | 102 | 23.429 | 2.695 | 26.110 | 1.00 | 30.05 | W | O |
| ATOM | 4807 | O | HOH | W | 103 | 5.939 | 23.666 | 15.058 | 1.00 | 45.24 | W | O |
| ATOM | 4810 | O | HOH | W | 104 | 15.157 | −8.657 | 41.812 | 1.00 | 48.42 | W | O |
| ATOM | 4813 | O | HOH | W | 105 | 22.584 | 5.351 | 6.685 | 1.00 | 32.02 | W | O |
| ATOM | 4816 | O | HOH | W | 106 | 1.947 | −13.531 | 12.995 | 1.00 | 35.70 | W | O |
| ATOM | 4819 | O | HOH | W | 107 | 0.843 | −3.343 | 5.954 | 1.00 | 35.00 | W | O |
| ATOM | 4822 | O | HOH | W | 108 | 10.764 | 8.978 | −0.078 | 1.00 | 47.97 | W | O |
| ATOM | 4825 | O | HOH | W | 109 | 12.609 | 11.593 | 33.799 | 1.00 | 33.88 | W | O |
| ATOM | 4828 | O | HOH | W | 110 | −2.559 | 28.085 | 29.916 | 1.00 | 40.24 | W | O |
| ATOM | 4831 | O | HOH | W | 111 | −8.695 | −8.532 | 8.837 | 1.00 | 37.60 | W | O |
| ATOM | 4834 | O | HOH | W | 112 | 1.265 | 18.134 | 33.506 | 1.00 | 37.44 | W | O |
| ATOM | 4837 | O | HOH | W | 113 | −9.981 | 6.869 | 11.619 | 1.00 | 38.18 | W | O |
| ATOM | 4840 | O | HOH | W | 114 | −2.744 | 14.784 | 38.949 | 1.00 | 50.37 | W | O |
| ATOM | 4843 | O | HOH | W | 115 | 20.301 | −12.723 | 34.212 | 1.00 | 40.49 | W | O |
| ATOM | 4846 | O | HOH | W | 116 | 14.851 | 9.838 | 20.023 | 1.00 | 37.48 | W | O |
| ATOM | 4849 | O | HOH | W | 117 | 23.651 | 3.698 | 39.134 | 1.00 | 37.78 | W | O |
| ATOM | 4852 | O | HOH | W | 118 | −18.557 | 13.257 | 30.128 | 1.00 | 41.89 | W | O |
| ATOM | 4855 | O | HOH | W | 119 | 18.487 | −21.421 | 23.859 | 1.00 | 39.29 | W | O |
| ATOM | 4858 | O | HOH | W | 120 | 21.377 | 4.874 | 37.522 | 1.00 | 28.87 | W | O |
| ATOM | 4861 | O | HOH | W | 121 | 18.415 | −7.751 | −2.248 | 1.00 | 36.44 | W | O |
| ATOM | 4864 | O | HOH | W | 122 | 17.602 | −7.842 | 12.411 | 1.00 | 16.38 | W | O |
| ATOM | 4867 | O | HOH | W | 123 | 16.678 | 4.872 | 18.751 | 1.00 | 22.46 | W | O |
| ATOM | 4870 | O | HOH | W | 124 | 14.066 | −0.309 | 37.545 | 1.00 | 30.70 | W | O |
| ATOM | 4873 | O | HOH | W | 125 | 7.740 | −4.673 | 37.771 | 1.00 | 27.15 | W | O |
| ATOM | 4876 | O | HOH | W | 126 | 13.728 | 4.405 | 15.630 | 1.00 | 25.76 | W | O |
| ATOM | 4879 | O | HOH | W | 127 | 27.821 | −6.501 | 23.920 | 1.00 | 31.77 | W | O |
| ATOM | 4882 | O | HOH | W | 128 | −3.928 | 27.852 | 25.640 | 1.00 | 29.97 | W | O |
| ATOM | 4885 | O | HOH | W | 129 | 16.702 | 6.462 | 21.069 | 1.00 | 30.52 | W | O |
| ATOM | 4888 | O | HOH | W | 130 | 26.185 | −5.646 | 20.509 | 1.00 | 24.01 | W | O |
| ATOM | 4891 | O | HOH | W | 131 | 1.974 | −3.227 | 0.396 | 1.00 | 29.45 | W | O |
| ATOM | 4894 | O | HOH | W | 132 | 18.503 | −17.071 | 10.260 | 1.00 | 34.39 | W | O |
| ATOM | 4897 | O | HOH | W | 133 | 10.397 | −13.616 | 1.651 | 1.00 | 39.32 | W | O |
| ATOM | 4900 | O | HOH | W | 134 | −2.831 | 28.209 | 21.200 | 1.00 | 30.33 | W | O |
| ATOM | 4903 | O | HOH | W | 135 | 15.320 | −22.101 | 24.023 | 1.00 | 25.19 | W | O |
| ATOM | 4906 | O | HOH | W | 136 | 4.007 | −9.460 | 34.087 | 1.00 | 38.19 | W | O |
| ATOM | 4909 | O | HOH | W | 137 | −1.239 | −11.368 | 25.339 | 1.00 | 28.01 | W | O |
| ATOM | 4912 | O | HOH | W | 138 | 25.810 | −13.282 | 12.651 | 1.00 | 29.26 | W | O |
| ATOM | 4915 | O | HOH | W | 139 | −11.471 | 3.486 | 23.719 | 1.00 | 43.71 | W | O |
| ATOM | 4918 | O | HOH | W | 140 | −14.572 | 6.189 | 16.547 | 1.00 | 32.73 | W | O |
| ATOM | 4921 | O | HOH | W | 141 | 25.455 | −4.517 | 28.979 | 1.00 | 27.27 | W | O |
| ATOM | 4924 | O | HOH | W | 142 | −2.265 | −18.449 | 18.806 | 1.00 | 31.12 | W | O |
| ATOM | 4927 | O | HOH | W | 143 | 24.881 | −12.339 | 32.109 | 1.00 | 47.48 | W | O |
| ATOM | 4930 | O | HOH | W | 144 | 3.783 | −12.994 | 33.207 | 1.00 | 40.92 | W | O |
| ATOM | 4933 | O | HOH | W | 145 | 16.661 | 4.952 | 10.161 | 1.00 | 41.86 | W | O |
| ATOM | 4936 | O | HOH | W | 146 | −9.286 | −1.522 | 21.390 | 1.00 | 35.03 | W | O |
| ATOM | 4939 | O | HOH | W | 147 | 14.410 | −11.209 | 45.987 | 1.00 | 39.95 | W | O |
| ATOM | 4942 | O | HOH | W | 148 | −1.544 | −13.594 | 23.703 | 1.00 | 33.58 | W | O |
| ATOM | 4945 | O | HOH | W | 149 | 4.387 | 7.952 | 19.690 | 1.00 | 35.86 | W | O |
| ATOM | 4948 | O | HOH | W | 150 | −8.699 | 18.279 | 6.531 | 1.00 | 32.48 | W | O |
| ATOM | 4951 | O | HOH | W | 151 | 3.719 | −3.684 | 38.401 | 1.00 | 30.50 | W | O |
| ATOM | 4954 | O | HOH | W | 152 | 3.775 | −20.312 | 14.291 | 1.00 | 42.65 | W | O |
| ATOM | 4957 | O | HOH | W | 153 | 27.957 | −13.614 | 22.778 | 1.00 | 37.15 | W | O |
| ATOM | 4960 | O | HOH | W | 154 | −6.363 | −9.621 | 22.860 | 1.00 | 31.35 | W | O |
| ATOM | 4963 | O | HOH | W | 155 | 17.426 | −9.113 | 39.788 | 1.00 | 41.82 | W | O |
| ATOM | 4966 | O | HOH | W | 156 | 11.719 | −0.755 | 38.894 | 1.00 | 44.37 | W | O |
| ATOM | 4969 | O | HOH | W | 157 | −1.823 | −18.279 | 23.964 | 1.00 | 31.03 | W | O |
| ATOM | 4972 | O | HOH | W | 158 | 1.236 | 19.857 | 5.620 | 1.00 | 33.82 | W | O |
| ATOM | 4975 | O | HOH | W | 159 | −5.447 | −2.279 | 8.958 | 1.00 | 43.06 | W | O |
| ATOM | 4978 | O | HOH | W | 160 | 6.495 | 1.870 | 6.024 | 1.00 | 32.59 | W | O |
| ATOM | 4981 | O | HOH | W | 161 | −1.902 | −10.079 | 30.428 | 1.00 | 40.56 | W | O |
| ATOM | 4984 | O | HOH | W | 162 | 12.781 | −10.832 | 0.891 | 1.00 | 28.70 | W | O |
| ATOM | 4987 | O | HOH | W | 163 | 15.705 | −18.496 | 12.332 | 1.00 | 42.03 | W | O |
| ATOM | 4990 | O | HOH | W | 164 | 11.477 | 23.027 | 19.258 | 1.00 | 43.18 | W | O |
| ATOM | 4993 | O | HOH | W | 165 | 11.794 | −19.441 | 25.110 | 1.00 | 34.30 | W | O |
| ATOM | 4996 | O | HOH | W | 166 | −9.142 | 6.492 | 24.586 | 1.00 | 37.55 | W | O |
| ATOM | 4999 | O | HOH | W | 167 | −3.791 | −17.095 | 17.004 | 1.00 | 40.54 | W | O |
| ATOM | 5002 | O | HOH | W | 168 | 18.861 | −5.381 | −3.135 | 1.00 | 38.29 | W | O |
| ATOM | 5005 | O | HOH | W | 169 | 1.506 | 21.043 | 36.589 | 1.00 | 44.96 | W | O |
| ATOM | 5008 | O | HOH | W | 170 | 12.593 | −2.711 | −7.043 | 1.00 | 44.98 | W | O |
| ATOM | 5011 | O | HOH | W | 171 | 15.127 | −0.179 | 0.329 | 1.00 | 35.60 | W | O |
| ATOM | 5014 | O | HOH | W | 172 | 15.056 | 2.474 | −0.998 | 1.00 | 37.93 | W | O |
| ATOM | 5017 | O | HOH | W | 173 | −7.283 | −6.499 | 7.178 | 1.00 | 49.30 | W | O |
| ATOM | 5020 | O | HOH | W | 174 | 21.316 | 0.314 | −2.427 | 1.00 | 41.75 | W | O |
| ATOM | 5023 | O | HOH | W | 175 | 26.651 | −5.640 | 2.963 | 1.00 | 36.35 | W | O |
| ATOM | 5026 | O | HOH | W | 176 | 29.087 | −10.247 | 25.287 | 1.00 | 39.54 | W | O |
| ATOM | 5029 | O | HOH | W | 177 | 23.713 | 5.560 | 13.541 | 1.00 | 39.25 | W | O |
| ATOM | 5032 | O | HOH | W | 178 | −1.768 | 14.984 | −1.759 | 1.00 | 40.11 | W | O |
| ATOM | 5035 | O | HOH | W | 179 | 13.927 | −22.767 | 20.107 | 1.00 | 44.51 | W | O |
| ATOM | 5038 | O | HOH | W | 180 | −5.833 | 17.092 | 42.432 | 1.00 | 39.14 | W | O |
| ATOM | 5041 | O | HOH | W | 181 | 4.780 | 28.120 | 30.190 | 1.00 | 35.76 | W | O |
| ATOM | 5044 | O | HOH | W | 182 | 5.232 | −21.397 | 10.130 | 1.00 | 32.74 | W | O |
| ATOM | 5047 | O | HOH | W | 183 | −0.609 | −13.905 | 29.169 | 1.00 | 42.77 | W | O |
| ATOM | 5050 | O | HOH | W | 184 | −8.615 | −12.423 | 20.365 | 1.00 | 38.88 | W | O |
| ATOM | 5053 | O | HOH | W | 185 | −2.742 | 24.869 | 15.943 | 1.00 | 35.44 | W | O |
| ATOM | 5056 | O | HOH | W | 186 | −6.914 | 23.870 | 13.421 | 1.00 | 38.92 | W | O |
| ATOM | 5059 | O | HOH | W | 187 | −9.844 | 5.148 | 9.425 | 1.00 | 47.06 | W | O |
| ATOM | 5062 | O | HOH | W | 188 | −3.377 | 1.908 | 31.500 | 1.00 | 47.62 | W | O |
| ATOM | 5065 | O | HOH | W | 189 | 8.535 | −2.717 | 9.847 | 1.00 | 20.99 | W | O |
| ATOM | 5068 | O | HOH | W | 190 | −2.120 | 23.920 | 11.635 | 1.00 | 33.73 | W | O |
| ATOM | 5071 | O | HOH | W | 191 | −1.821 | 12.406 | 36.174 | 1.00 | 33.31 | W | O |
| ATOM | 5074 | O | HOH | W | 192 | 15.430 | −22.456 | 17.980 | 1.00 | 32.67 | W | O |
| ATOM | 5077 | O | HOH | W | 193 | 26.766 | −7.447 | 10.363 | 1.00 | 37.80 | W | O |
| ATOM | 5080 | O | HOH | W | 194 | 7.871 | −12.672 | 1.551 | 1.00 | 33.24 | W | O |
| ATOM | 5083 | O | HOH | W | 195 | 11.658 | −13.419 | −1.004 | 1.00 | 38.16 | W | O |
| ATOM | 5086 | O | HOH | W | 196 | 16.826 | 1.526 | 0.614 | 1.00 | 50.30 | W | O |
| ATOM | 5089 | O | HOH | W | 197 | 15.595 | 6.152 | 14.916 | 1.00 | 40.35 | W | O |
| ATOM | 5092 | O | HOH | W | 198 | −1.881 | 25.715 | 18.176 | 1.00 | 37.84 | W | O |
| ATOM | 5095 | O | HOH | W | 199 | −11.651 | 11.014 | 33.297 | 1.00 | 32.46 | W | O |
| ATOM | 5098 | O | HOH | W | 200 | 18.893 | −2.239 | 0.202 | 1.00 | 36.54 | W | O |
| ATOM | 5101 | O | HOH | W | 201 | 8.083 | −15.775 | 41.296 | 1.00 | 35.79 | W | O |
| ATOM | 5104 | O | HOH | W | 202 | 27.247 | −8.234 | 2.554 | 1.00 | 45.23 | W | O |
| ATOM | 5107 | O | HOH | W | 203 | −1.222 | −15.328 | 27.055 | 1.00 | 46.27 | W | O |
| ATOM | 5110 | O | HOH | W | 204 | 13.756 | −2.002 | 41.227 | 1.00 | 44.19 | W | O |
| ATOM | 5113 | O | HOH | W | 205 | 18.212 | 11.234 | 28.397 | 1.00 | 46.27 | W | O |
| ATOM | 5116 | O | HOH | W | 206 | 10.446 | 24.528 | 22.495 | 1.00 | 29.18 | W | O |
| ATOM | 5119 | O | HOH | W | 207 | 9.812 | 7.702 | 16.580 | 1.00 | 47.51 | W | O |
| ATOM | 5122 | O | HOH | W | 208 | 8.614 | 13.189 | 26.746 | 1.00 | 42.96 | W | O |
| ATOM | 5125 | O | HOH | W | 209 | 26.242 | 4.872 | 13.201 | 1.00 | 34.36 | W | O |
| ATOM | 5128 | O | HOH | W | 210 | 3.417 | 32.021 | 7.569 | 1.00 | 43.78 | W | O |
| ATOM | 5131 | O | HOH | W | 211 | 13.082 | 12.607 | −0.030 | 1.00 | 38.29 | W | O |
| ATOM | 5134 | O | HOH | W | 212 | 11.113 | −16.047 | 1.534 | 1.00 | 48.53 | W | O |
| ATOM | 5137 | O | HOH | W | 213 | 16.799 | −19.980 | 17.140 | 1.00 | 42.01 | W | O |
| ATOM | 5140 | O | HOH | W | 214 | 8.100 | 2.699 | 9.516 | 1.00 | 38.54 | W | O |
| ATOM | 5143 | O | HOH | W | 215 | 21.192 | −10.596 | 35.721 | 1.00 | 42.09 | W | O |
| ATOM | 5146 | O | HOH | W | 216 | −12.311 | 27.355 | 25.513 | 1.00 | 46.88 | W | O |
| ATOM | 5149 | O | HOH | W | 217 | 2.196 | 18.838 | 8.312 | 1.00 | 40.97 | W | O |
| ATOM | 5152 | O | HOH | W | 218 | 2.760 | 7.802 | 34.907 | 1.00 | 40.65 | W | O |
| ATOM | 5155 | O | HOH | W | 219 | 2.052 | 34.572 | 8.653 | 1.00 | 28.96 | W | O |
| ATOM | 5158 | O | HOH | W | 220 | 20.199 | 10.058 | 26.993 | 1.00 | 39.00 | W | O |
| ATOM | 5161 | O | HOH | W | 221 | 0.666 | 6.917 | 33.693 | 1.00 | 41.05 | W | O |
| ATOM | 5164 | O | HOH | W | 222 | 19.656 | −20.516 | 17.448 | 1.00 | 49.29 | W | O |
| ATOM | 5167 | O | HOH | W | 223 | 24.529 | −13.997 | 28.898 | 1.00 | 44.80 | W | O |
| ATOM | 5170 | O | HOH | W | 224 | −15.502 | 8.924 | 35.463 | 1.00 | 46.22 | W | O |
| ATOM | 5173 | O | HOH | W | 225 | 6.321 | 6.726 | 39.047 | 1.00 | 40.39 | W | O |
| ATOM | 5176 | O | HOH | W | 226 | 13.346 | −19.882 | 30.564 | 1.00 | 27.35 | W | O |
| ATOM | 5179 | O | HOH | W | 227 | 2.475 | 8.509 | 20.962 | 1.00 | 37.71 | W | O |
| ATOM | 5182 | O | HOH | W | 228 | 25.697 | −17.409 | 7.650 | 1.00 | 40.93 | W | O |
| ATOM | 5185 | O | HOH | W | 229 | −5.326 | 22.902 | 36.076 | 1.00 | 34.85 | W | O |
| ATOM | 5188 | O | HOH | W | 230 | 18.689 | 1.894 | 1.969 | 1.00 | 40.05 | W | O |
| ATOM | 5191 | O | HOH | W | 231 | 22.256 | 9.449 | 29.382 | 1.00 | 38.70 | W | O |
| ATOM | 5194 | O | HOH | W | 232 | 6.671 | 16.029 | 32.045 | 1.00 | 45.79 | W | O |
| ATOM | 5197 | O | HOH | W | 233 | −12.901 | 11.354 | 24.852 | 1.00 | 42.77 | W | O |
| ATOM | 5200 | O | HOH | W | 234 | 11.146 | −21.564 | 4.017 | 1.00 | 44.94 | W | O |
| ATOM | 5203 | O | HOH | W | 235 | 25.034 | −1.313 | 25.290 | 1.00 | 35.63 | W | O |
| ATOM | 5206 | O | HOH | W | 236 | −14.460 | 18.497 | 19.730 | 1.00 | 43.85 | W | O |
| ATOM | 5209 | O | HOH | W | 237 | −12.580 | 11.671 | 30.829 | 1.00 | 49.50 | W | O |
| ATOM | 5212 | O | HOH | W | 238 | −15.352 | 9.953 | 23.232 | 1.00 | 41.57 | W | O |
| ATOM | 5215 | O | HOH | W | 239 | −1.668 | 11.121 | 33.395 | 1.00 | 56.23 | W | O |
| ATOM | 5218 | O | HOH | W | 240 | 8.754 | −13.126 | −3.263 | 1.00 | 52.79 | W | O |
| ATOM | 5221 | O | HOH | W | 241 | −1.876 | −3.651 | 6.368 | 1.00 | 34.87 | W | O |
| ATOM | 5224 | O | HOH | W | 242 | 19.512 | −18.187 | 6.893 | 1.00 | 42.20 | W | O |
| ATOM | 5227 | O | HOH | W | 243 | −8.077 | 30.761 | 18.011 | 1.00 | 33.22 | W | O |
| ATOM | 5230 | O | HOH | W | 244 | 11.861 | 14.174 | 19.992 | 1.00 | 49.09 | W | O |
| ATOM | 5233 | O | HOH | W | 245 | 0.301 | 11.850 | −6.763 | 1.00 | 46.36 | W | O |
| ATOM | 5236 | O | HOH | W | 246 | −2.640 | 12.555 | 0.451 | 1.00 | 48.51 | W | O |
| ATOM | 5239 | O | HOH | W | 247 | 12.588 | −19.822 | 12.376 | 1.00 | 48.93 | W | O |
| ATOM | 5242 | O | HOH | W | 248 | 11.738 | 20.944 | 27.362 | 1.00 | 51.67 | W | O |
| ATOM | 5245 | O | HOH | W | 249 | 17.071 | 8.568 | 19.617 | 1.00 | 43.62 | W | O |
| ATOM | 5248 | O | HOH | W | 250 | −2.350 | −17.778 | 11.732 | 1.00 | 51.64 | W | O |
| ATOM | 5251 | O | HOH | W | 251 | 12.306 | −4.700 | 40.223 | 1.00 | 48.28 | W | O |
| END | | | | | | | | | | | | |

[image] TABLE 4
PYK2 in pET15S

U33284; Human protein tyrosine kinase PYK2 mRNA, complete cds
Full-length protein in pET15S: 293 aa (SEQ ID NO: 2)
Mass: 33872.2 pI: 6.07
PYK2 kinase domain I420-M691 (not including first 21 aa in
following sequence) SEQ ID NO: 1

| | |
|---|---|
| 1 | MGSSHHHHHH SSGLVPRGSH MIAREDVVLN RILGEGFFGE VYEGVYTNHK GEKINVAVKT |
| 61 | CKKDCTLDNK EKFMSEAVIM KNLDHPHIVK LIGIIEEEPT WIIMELYPYG ELGHYLERNK |
| 121 | NSLKVLTLVL YSLQICKAMA YLESINCVHR DIAVRNILVA SPECVKLGDF GLSRYIEDED |
| 181 | YYKASVTRLP IKWMSPESIN FRRFTTASDV WMFAVCMWEI LSFGKQPFFW LENKDVIGVL |
| 241 | EKGDRLPKPD LCPPVLYTLM TRCWDYDPSD RPRFTELVCS LSDVYQMEKD IAM |

| | | |
|---|---|---|
| SEQ ID NO: 5 | | |
| PYK2-C1; | 5′-TCCACAG CATATG ATTGCCCGTGAAGATGTGGT-3′ | 33 mer |
| SEQ ID NO: 6 | | |
| PYK2-N2; | TGGAGAAGGACATTGCCATG TAG GTCGAC GAGAG (Origin) | |
| | 5′-CTCTC GTCGAC CTA CATGGCAATGTCCTTCTCCA-3′ | 34 mer |

pET15S sequence PCR product; 843 bp (SEQ ID NO: 4)
Sequence encoding PYK2 kinase domain (in small letters below)
(SEQ ID NO: 3)

TCTAGAAATAATTTTGTTTAACTTTAAGAAGGAGATATACCATGGGCAGCAGCCATCATCATCATCAT
CACAGCAGCGGCCTGGTGCCGCGCGGCAGCCATATG

| | |
|---|---|
| | attgcc cgtgaagatg |
| 1381 | tggtcctgaa tcgtattctt ggggaaggct tttttgggga ggtctatgaa ggtgtctaca |
| 1441 | caaatcacaa aggggagaaa atcaatgtag ctgtcaagac ctgcaagaaa gactgcactc |
| 1501 | tggacaacaa ggagaagttc atgagcgagg cagtgatcat gaagaacctc gaccacccgc |
| 1561 | acatcgtgaa gctgatcggc atcattgaag aggagcccac ctggatcatc atggaattgt |
| 1621 | atccctatgg ggagctgggc cactacctgg agcggaacaa gaactccctg aaggtgctca |
| 1681 | ccctcgtgct gtactcactg cagatatgca aagccatggc ctacctggag agcatcaact |
| 1741 | gcgtgcacag ggacattgct gtccggaaca tcctggtggc ctcccctgag tgtgtgaagc |
| 1801 | tgggggactt tggtctttcc cggtacattg aggacgagga ctattacaaa gcctctgtga |
| 1861 | ctcgtctccc catcaaatgg atgtccccag agtccattaa cttccgacgc ttcacgacag |
| 1921 | ccagtgacgt ctggatgttc gccgtgtgca tgtgggagat cctgagcttt gggaagcagc |
| 1981 | ccttcttctg gctggagaac aaggatgtca tcggggtgct ggagaaagga gaccggctgc |
| 2041 | ccaagcctga tctctgtcca ccggtccttt ataccctcat gacccgctgc tgggactacg |
| 2101 | accccagtga ccggccccgc ttcaccgagc tggtgtgcag cctcagtgac gtttatcaga |
| 2161 | tggagaagga cattgccatg |

TAGGTCGACTAGAGCCTGCAGTCTCGACCATCATCATCATCATCATTAATAAAAGGGCGAATTCCAG
CACACTGGCGGCCGTTACTAGTGGATCCGGCTGCTAACAAAGCCCGAAAGGAAGCTGAGTTGG

TABLE 5
Pyk2 Activity and the Inhibition by ATP Analogs

| Pyk2 | Vmax | Vmax (SE) | K | K (SE) | K (Lo 95%) | K (Up 95%) | Equation |
|---|---|---|---|---|---|---|---|
| 8 ng/well | 1.25e+4 | 9.11e+2 | 7.37e+0 | 2.79e+0 | 3.27e+0 | 1.66e+1 | y = (Vmax * x)/(K + x) |

| Compounds | Vmax | K | K (SE) | K (Lo 95%) | K (Up 95%) | Y2 | n | Equation |
|---|---|---|---|---|---|---|---|---|
| Adenosine | 1.82e+4 | 2.54e+2 | 2.65e+2 | 2.47e+1 | 2.60e+3 | 7.33e+2 | −5.14e−1 | y = ((Vmax * x\hat{ }n)/(K\hat{ }n + x\hat{ }n)) + Y2 |
| AMP | 1.82e+4 | 8.02e+1 | 3.76e+1 | 2.82e+1 | 2.28e+2 | 7.33e+2 | −5.05e−1 | y = ((Vmax * x\hat{ }n)/(K\hat{ }n + x\hat{ }n)) + Y2 |
| ADT | 1.82e+4 | 1.49e+1 | 2.69e+0 | 9.93e+0 | 2.22e+1 | 7.33e+2 | −7.69e−1 | y = ((Vmax * x\hat{ }n)/(K\hat{ }n + x\hat{ }n)) + Y2 |
| AMPPCP | 1.82e+4 | 7.69e+3 | 1.99e+4 | 2.43e+1 | 2.44e+6 | 7.33e+2 | −2.03e−1 | y = ((Vmax * x\hat{ }n)/(K\hat{ }n + x\hat{ }n)) + Y2 |
| AMPPNP | 1.82e+4 | 1.81e+1 | 2.82e+0 | 1.28e+1 | 2.56e+1 | 7.33e+2 | −7.18e−1 | y = ((Vmax * x\hat{ }n)/(K\hat{ }n + x\hat{ }n)) + Y2 |
| ATP-g-S | 1.82e+4 | 1.36e+1 | 1.49e+0 | 1.06e+1 | 1.73e+1 | 7.33e+2 | −9.66e−1 | y = ((Vmax * x\hat{ }n)/(K\hat{ }n + x\hat{ }n)) + Y2 |

1. A method for identifying compounds binding to PYK2, comprising
determining the orientation of at least one compound bound with PYK2 in co-crystals of PYK2 with said compound.

2. The method of claim 1, wherein said compound is identified as a molecular scaffold if it binds weakly to PYK2 and has a molecular weight less than 350 daltons.

3. The method of claim 2, wherein said method further comprises identifying chemical structures of said molecular scaffolds, that, when modified, alter the binding affinity or binding specificity or both between the molecular scaffold and PYK2.

4. The method of claim 3, wherein further comprising synthesizing a ligand wherein one or more of the chemical structures of the molecular scaffold is modified to provide a ligand that binds to PYK2 with altered binding affinity or binding specificity or both.

5. The method of claim 2, wherein said molecular scaffold binds to a plurality of kinases.

6. The method of claim 2, wherein said molecular scaffold interacts with one or more of PYK2 residues 503, 505, 457, 488, 567, and 554.

7. The method of claim 4, wherein said ligand has a chemical structure of Formula

8. A method for obtaining improved ligands binding to PYK2, comprising
identifying a compound that binds to PYK2 and determining whether said compound interacts with one or more of PYK2 residues 503, 505, 457, 488, 567, and 554;
determining whether a derivative of said compound binds to PYK2 with greater affinity or greater specificity or both than said compound, wherein binding with greater affinity or greater specificity or both indicates that said derivative is an improved ligand.

9. The method of claim 8, wherein said derivative has at least 10-fold greater affinity or specificity or both than said compound.

10. The method of claim 8, wherein said derivative has at least 100-fold greater affinity or specificity or both.

11. The method of claim 8, wherein said compound has a chemical structure of Formula I.

12. A method for developing ligands specific for PYK2, comprising
identifying a compound that binds to a plurality of kinases; and
determining whether a derivative of said compound has greater specificity for PYK2 than said compound.

13. The method of claim 12, wherein said compound binds to PYK2 with an affinity at least 10-fold greater than for binding to any of said plurality of kinases.

14. The method of claim 12, wherein said compound interacts with at least one of PYK2 residues residues 503, 505, 457, 488, 567, and 554.

15. The method of claim 12, wherein said compound is a compound of Formula I.

16. The method of claim 12, wherein said compound binds weakly to said plurality of kinases.

17. A crystalline form of PYK2 kinase domain.

18. The crystalline form of claim 17, having coordinates as described in Table 1.

19. The crystalline form of claim 17, comprising one more more heavy metal atoms.

20. The crystalline form of claim 17, wherein said crystalline form comprises a co-crystal of PYK2 with a binding compound.

21. The crystalline form of claim 20, wherein said binding compound interacts with one or more of PYK2 residues 503, 505, 457, 488, 567, and 554.

22. The crystalline form of claim 17, wherein said crystalline form is in an X-ray beam.

23. The crystalline form of claim 17, wherein said PYK2 is mutated.

24. A method for obtaining a crystal of PYK2, comprising subjecting PYK2 protein at 5-20 mg/ml to crystallization condition substantially equivalent to 2-10% polyethylene glycol (PEG) 8000, 0.2 M sodium acetate, 0.1% sodium cacodylate pH 6.5, 20% glycerol for a time sufficient for cystal development.

25. The method of claim 24, further comprising optimizing said crystallization condition.

26. The method of claim 24, wherein said crystallization condition comprises approximately 8% polyethylene glycol (PEG) 8000.

27. The method of claim 24, wherein said PYK2 is seleno-methionine labeled PYK2.

28. A co-crystal of PYK2 and a PYK2 binding compound.

29. The co-crystal of claim 28, wherein said binding compound interacts with at least one of PYK2 residues 503, 505, 457, 488, 567, and 554.

30. The co-crystal of claim 28, wherein said binding compound has a chemical structure of Formula I.

31. The co-crystal of claim 28, wherein said co-crystal is in an X-ray beam.

32. A method for obtaining co-crystals of PYK2 with a binding compound, comprising subjecting PYK2 protein at 5-20 mg/ml to crystallization conditions 2-10% polyethylene glycol (PEG) 8000, 0.2 M sodium acetate, 0.1% sodium cacodylate pH 6.5, 20% glycerol in the presence of binding compound for a time sufficient for cystal development.

33. The method of claim 32, wherein said binding compound is added to said protein to a final concentration of 0.5 to 1.0 mM.

34. The method of claim 32, wherein said binding compound is in a dimethyl sulfoxide solution.

35. The method of claim 32, wherein said crystallization condition comprise approximately 8% polyethylene glycol (PEG) 8000.

36. A method for determining a structure of a kinase, comprising
creating a homology model from an electronic representation of a PYK2 structure.

37. The method of claim 36, wherein said creating comprises
identifying conserved amino acid residues between PYK2 and said kinase;
transferring the atomic coordinates of a plurality of conserved amino acids in said PYK2 structure to the corresponding amino acids of said kinase to provide a rough structure of said kinase; and
constructing structures representing the remainder of said kinase using electronic representations of the structures of the remaining amino acid residues in said kinase.

38. The method of claim 37, further comprising fitting said homology model to low resolution x-ray diffraction data from one or more crystals of said kinase.

39. The method of claim 37, wherein the coordinates of conserved residues from Table 3 are utilized.

40. The method of claim 37, wherein coordinates of conserved residues from a mutated PYK2 are utilized.

41. An electronic representation of a crystal structure of PYK2.

42. The electronic representation of claim 41, containing atomic coordinate representations corresponding to the coordinates listed in Table 1 or Table 2.

43. The electronic representation of claim 41, comprising a schematic representation.

44. The electronic representation of claim 41, wherein atomic coordinates for a mutated PYK2 are utilized.

45. The electronic representation of claim 44, wherein said PYK2 consists essentially of a PYK2 kinase domain.

46. An electronic representation of a binding site of PYK2.

47. The electronic representation of claim 46, comprising representations of PYK2 residues 503, 505, 457, 488, 567, and 554.

48. The electronic representation of claim 46, comprising a binding site surface contour.

49. The electronic representation of claim 46, comprising representations of the binding character of a plurality of conserved amino acid residues.

50. The electronic representation of claim 46, further comprising an electronic representation of a binding compound in a binding site of PYK2.

51. The electronic representation of claim 46, wherein said PYK2 is a mutated PYK2.

52. An electronic representation of a PYK2 based homology model for a kinase.

53. The electronic representation of claim 52, wherein said homology model utilizes conserved residue atomic coordinates of Table 1 or Table 2.

54. The electronic representation of claim 52, wherein atomic coordinates for a mutated PYK2 are utilized.

55. A method for identifying a ligand binding to PYK2, comprising
determining whether a derivative compound that includes a core structure of Formula I binds to PYK2 with altered binding affinity or specificity or both as compared to the parent compound.

56. A method for modulating PYK2 activity, comprising
contacting PYK2 with a compound that binds to PYK2 and interacts with three or more of residues 503, 505, 457, 488, 567, and 554.

57. The method of claim 56, wherein said compound is a compound of Formula I.

58. The method of claim 56, wherein said compound is at a concentration of 200 μM or less.

59. A method for treating a patient suffering from a disease or condition characterized by abnormal PYK2 activity, comprising
administering to said patient a compound that interacts with three or more of PYK2 residues 503, 505, 457, 488, 567, and 554.

60. The method of claim 59, wherein said compound is a compound of Formula I.

61. The method of claim 59 wherein said disease or condition is a cancer.

62. The method of claim 59, wherein said disease or condition is an inflammatory disease or condition.

63. The method of claim 59, wherein said compound interacts with residues 503 and 505.

64. An electronic representation of a modified PYK2 crystal structure, comprising
an electronic representation of the atomic coordinates of a modified PYK2.

65. The electronic representation of claim 64, wherein said modified PYK2 comprises a C-terminal deletion, an N-terminal deletion or both.

66. A method for developing a biological agent, comprising
analyzing a PYK2 crystal structure and identifying at least one sub-structure for forming a said biological agent.

67. The method of claim 66, wherein said substructure comprises an epitope, and said method further comprises developing antibodies against said epitope.

68. The method of claim 66, wherein said sub-structure comprises a mutation site expected to provide altered activity, and said method further comprises creating a mutation at said site thereby providing a modified PYK2.

69. The method of claim 66, wherein said sub-structure comprises an attachment point for attaching a separate moiety.

70. The method of claim 69, wherein said separate moiety is selected from the group consisting of a peptide, a polypeptide, a solid phase material, a linker, and a label.

71. The method of claim 69, further comprising attaching said separate moiety.

72. A method for identifying potential PYK2 binding compounds, comprising
fitting at least one electronic representation of a compound in an electronic representation of a PYK2 binding site.

73. The method of claim 72, wherein said electronic representation of a PYK2 binding site is defined by atomic structural coordinates set forth in Table 1 or Table 2.

74. The method of claim 73, comprising
removing a computer representation of a compound complexed with PYK2 and fitting a computer representation of a compound from a computer database with a computer representation of the active site of PYK2; and
identifying compounds that best fit said active site based on favorable geometric fit and energetically favorable complementary interactions as potential binding compounds.

75. The method of claim 73, comprising
modifying a computer representation of a compound complexed with PYK2 by the deletion or addition or both of one or more chemical groups;
fitting a computer representation of a compound from a computer database with a computer representation of the active site of PYK2; and
identifying compounds that best fit said active site based on favorable geometric fit and energetically favorable complementary interactions as potential binding compounds.

76. The method of claim 73, comprising
removing a computer representation of a compound complexed with PYK2 and; and
searching a database for compounds having structural similarity to said compound using a compound searching computer program or replacing portions of said compound with similar chemical structures using a compound construction computer program.

77. The method of claim 73, wherein said compound is a compound of Formula I.

78. The method of claim 82, wherein said fitting comprises determining whether a said compound will interact with one or more of PYK2 residues 503, 505, 457, 488, 567, and 554.

79. A method for attaching a PYK2 binding compound to an attachment component, comprising
identifying energetically allowed sites for attachment of a said attachment component on a kinase binding compound; and
attaching said compound or derivative thereof to said attachment component at said energetically allowed site.

80. The method of claim 79, wherein said attachment component is a linker for attachement to a solid phase medium, and said method further comprises attaching said compound or derivative to a solid phase medium through a linker attached at a said energetically allowed site.

81. The method of claim 79, wherein said kinase comprises conserved residues matching at least one of PYK2 residues 503, 505, 457, 488, 567, and 554.

82. The method of claim 80, wherein said linker is a traceless linker.

83. The method of claim 80, wherein said kinase binding compound or derivative thereof is synthesized on a said linker attached to said solid phase medium.

84. The method of claim 83, wherein a plurality of said compounds or derivatives are synthesized in combinatorial synthesis.

85. The method of claim 80, wherein attachment of said compound to said solid phase medium provides an affinity medium.

86. The method of claim 79, wherein said attachment component comprises a label.

87. The method of claim 86, wherein said label comprises a fluorophore.

88. A modified compound, comprising
a compound of Formula I, with a linker moiety attached thereto at an energetically allowed site for binding of said modified compound to PYK2.

89. The compound of claim 88, whereins said linker is attached to a solid phase.

90. The compound of claim 88, wherein said linker comprises or is attached to a label.

91. The compound of claim 88, wherein said linker is a traceless linker.

92. A method for developing a ligand for a kinase comprising conserved residues matching one or more of PYK2 residues residues 503, 505, 457, 488, 567, and 554, comprising
determining whether a compound of Formula I binds to said kinase and interacts with said residue.

93. The method of claim 92, wherein said kinase comprises conserved residues matching at least 2 of PYK2 residues 503, 505, 457, 488, 567, and 554.

94. The method of claim 92, wherein said kinase comprises conserved residues matching PYK2 residues 503, 505, 457, 488, 567, and 554.

95. The method of claim 92, further comprising determining whether said compound modulates said kinase.

96. The method of claim 92, wherein said determining comprises computer fitting said compound in a binding site of said kinase.

97. The method of claim 92, further comprising forming a co-crystal of said kinase and said compound.

98. The method of claim 97, further comprising determining the binding orientation of said compound with said kinase.
